SEQUENCE LISTING (924 sequences)

SEQ ID NO: 1 - RNA, 386 nt, Artificial Sequence
  Synthetic Construct
ccccauaggguggugugugccaccccugaugagaccgaaaggucgaaaugggguuucgccaugggucgggacuuuaaauucggaggauucguccuuuaaacguuccuccaagagucccuuccccaaacccuuacuuuguaagugugguucggcgaauguaccguuucguccuuucggacucaucagggaaaguacacacuuuccgacgguggguucgucgacaccucucccccucccagguacuauccccuuucaaggauguguucccuaggaggguggguguaccucuuuuggauugcuccggccuuccaggagagauagaggacgaccucuccccauaggguggugugugccaccccugaugagaccgaaaggucgaaaugggg

SEQ ID NO: 2 - RNA, 89 nt, Artificial Sequence
  Synthetic Construct
ugugauaaauguacauauaugguuugacagaaaacugaaguucaagagacuucaguuuucugucaaaccauauauguacauuuaucaca

SEQ ID NO: 3 - RNA, 475 nt, Artificial Sequence
  Synthetic Construct
ggguggugugugccaccccugaugagaccgaaaggucgaaaugggguuucgccaugggucgggacuuuaaauucggaggauucguccuuuaaacguuccuccaagagucccuuccccaaacccuuacuuuguaagugugguucggcgaauguaccguuucguccuuucggacucaucagggaaaguacacacuuuccgacgguggguucgucgacaccucucccccucccagguacuauccccuuugugauaaauguacauauaugguuugacagaaaacugaaguucaagagacuucaguuuucugucaaaccauauauguacauuuaucacaucaaggauguguucccuaggaggguggguguaccucuuuuggauugcuccggccuuccaggagagauagaggacgaccucuccccauaggguggugugugccaccccugaugagaccgaaaggucgaaauggggccccaua

SEQ ID NO: 4 - RNA, 63 nt, Artificial Sequence
  Synthetic Construct
uaauuucuacucuuguagaugucacagaucacaaacuucaaauuaauuucuacucuuguagau

SEQ ID NO: 5 - RNA, 449 nt, Artificial Sequence
  Synthetic Construct
ggguggugugugccaccccugaugagaccgaaaggucgaaaugggguuucgccaugggucgggacuuuaaauucggaggauucguccuuuaaacguuccuccaagagucccuuccccaaacccuuacuuuguaagugugguucggcgaauguaccguuucguccuuucggacucaucagggaaaguacacacuuuccgacgguggguucgucgacaccucucccccucccagguacuauccccuuuaauuucuacucuuguagaugucacagaucacaaacuucaaauuaauuucuacucuuguagauucaaggauguguucccuaggaggguggguguaccucuuuuggauugcuccggccuuccaggagagauagaggacgaccucuccccauaggguggugugugccaccccugaugagaccgaaaggucgaaauggggccccaua

SEQ ID NO: 6 - DNA, 3040 bp, Artificial Sequence
  Synthetic Construct
ccattacccgcccgcccgccacaggccacatcgtttccggccctgctcggctatatatccgcgagcatctgcatatcgccatatccccgccccgggcaccgcgagctagctagctactgacacccggcgccggcgccgagtacaatacaaggaaagcagcatggtgttcgagcagcatgaggaggaggcggtggcgccgggcgcggtgcacgggcaccggctctccacggtggtgccgagctcggtgacgggggaggtggactacgcgctggcggacgcggacctggccttcaagctgcactacctgcgcggggtgtactactaccggtccggggacggcctcgccaccaaggtgctcaaggaccccatgttcccgtggctggacgaccacttccccgtggccggccgcgtccgccgcgccgaggcggagggggacggcgcgccgcgccggccctacatcaagtgcaacgactgcggcgtgcgcatcgtggaggccaggtgcgaccgcgacatggccgagtggatccgcgacgccgcgcccggcaggatcaggcagctctgctacgacaaggtgctcggccccgagctcttcttctcgccgctgctctacgtccaggtaaccgtcctccgtacgtcgtcgtagagaggtggagatttttgtggtcggatttctggcatcgctggttgcctgctcccagaccagtccctgctggtgacggcaggagaggagacgcatgcaggctgtagctttcggtatggttacgggcagtgcagttgagcaagcttttaattgtggtgcggcagtagtacctgctaaccaccatcagacatttatactgactgtccagtggccacctagagctggtcaccgactgcaaccccccgtccccgtgtcaccatcatgccacaactgtgtagatagtgtctgtcgacccggctgtcaccactactactagttcagactggaccgcttctctgaacaagcagttaagtacccccattcatctgccggctcatgaaagacggcggcgggttcgtcgtgagactgtcgggtcggtgtcgcacgagtaattgctacgccggggcgggatggcgtactcccattcatctgccgccgcccacgttgaagcgggcgcttgggaccaaagatcagcacccacagccttgcaatcaattcactccctcgtagtactacctagctctgacataaaaactagtacagtaccattgaagccgatagccgatagattggaatattgatgtcacgatttagtatcagtgagaagatcatcaaagcccaagcagtacacgaagaatgggccagcaccagtcggcatagggtttatggatcgcctccctggcgggtggtgcattacgatctgccttttttcatgtaggcccaatgcgttggttgcattattgtctcacttgtttacccaaatagtactactctactactactagtacccacatagcacgtacggagtatgaagtcaatcaaaggtgcaaatcacggctttgtatactagactagtctattacagtacagacaattactgcaaactaattgtgctggtcgattacttctgttactaagggcatgtacaacttagatacaactgcacggtactccaagtataagacacaactaaaacacaatataatacagtggtcatgtctaaaacatgtgtcttaccatatttattgtaccaatcagggcattcaataaattaaagtgaccaatcagatagtctcatgtctcgaatatagagctaagacaccgtgtcttcgtcaaaatacatgtcttgagattttttacattcaccctcctagacacactctaagatacaatttaagacaccccacggtacatgccctaactacgtactccctccgtccttttttatttatcgtttctttggtcacagatcacaaacttcaaatgcggtgggctggcgctggggttcagctgggcgcacctcatcggcgacatcccgtcggccgccacctgcttcaacaagtgggcgcagatcctcagcggcaagaagccggaagccaccgtcctcaccccgccgaaccagccgctgcagggccagtcccccgcggcgccgcgctccgtcaagcaggtcgggcccatggaggacctctggctggtccccgcgggccgcgacatggcgtgctactccttccacgtcagcgacgcggtgctcaagaagctccaccagcagcagaatgggcgccaggacgcggccgctggcaccttcgagctcgtgtcggcgctggtgtggcaggcggtggccaagatcaggggcgacgtggacaccgtgaccgtggtcagggccgacgctgccgcgcggagcggcaagtcgctggccaacgagatgaaggtcgggtacgtggagtcggcggggtcgtcgccggcgaagacggacgtcgcggagctggcggcgctgctggccaagaacgtggtcgacgagaccgccgcggtggcggcgttccagggggacgtgctcgtgtacggcggcgccaacctgacgctcgtggacatggagcaggtggacctgtacgggctcgagatcaaggggcagcgcccggtctacgtggagtacggcatggacggcgtcggcgacgagggcgcggtgctggtgcagcccgacgccgacgggcgcggccgcctcgtcacggtggttttgcccggcgacgagatcgacagcctccgcgccgcgctcggtagcgcgctgcacgtcgcctgaaagctctagtacgtatcataatagtacgctgctgcctacgtgctcgttgcgtcgttcggtttcgcgatatcagagtaccacctcttgaagaaagtgtagtagcgtaatgaggcgtgactagtgatgtgatgtattgtactgtattgcaggatgttgcgctaatactgtggtcgatttgcgatgtttgttctgtatcggatgatgtttccttaataagctgctcgaatagatcatgctcaatgtaccattatttacactggagactagacaaaaacaatcgtttgata

SEQ ID NO: 7 - RNA, 20 nt, Artificial Sequence
  Synthetic Construct
uaauuucuacucuuguagau

SEQ ID NO: 8 - RNA, 359 nt, Artificial Sequence
  Synthetic Construct
cggaacuaaacucgugguuccugugguucacaccugaccuccugacaagaaaagaaaaaagaaggcggcucggaggagcgcuucagggauccccggggaaaccuggagcgaacuggcaaaaaaggacgguggggagugcccagcggccgacaggaguaauucccgccgaaacaggguuuucacccuuccuuucuucggguguccuuccucgcgcccgcaggaccaccccucgcccccuuugcgcugucgcuucggcuacuacccgguggaaacaacugaagcucccgagaaccgcuuuuucucuaucuuacuugcuccggggcgaggguguuuagcccuuggaaccgcaguugguuccu

SEQ ID NO: 9 - RNA, 21 nt, Artificial Sequence
  Synthetic Construct
cuucaguuuucugucaaacca

SEQ ID NO: 10 - RNA, 359 nt, Artificial Sequence
  Synthetic Construct
cggaacuaaacucgugguuccugugguucacaccugaccuccugacaagaaaagaaaaaagaaggcggcucggaggagcgcuucagggauccccggggaaaccuggagcgaacuggcaaaaaaggacgguggggagugcccagcggccgacaggaguaauucccgccgaaacaggguuuucacccuuccucuucaguuuucugucaaaccacgcccgcaggaccaccccucgcccccuuugcgcugucgcuucggcuacuacccgguggaaacaacugaagcucccgagaaccgcuuuuucucuaucuuacuugcuccggggcgaggguguuuagcccuuggaaccgcaguugguuccu

SEQ ID NO: 11 - RNA, 67 nt, Artificial Sequence
  Synthetic Construct
gguguuuagcccuuggaaccgcaguugguuccucggaacuaaacucgugguuccugugguucacacc

SEQ ID NO: 12 - RNA, 218 nt, Artificial Sequence
  Synthetic Construct
cuucggcuacuacccgguggaaacaacugaagcucccgagaaccgcuuuuucucuaucuuacuugcuucggggcgaggguguuuagcccuuggaaccgcaguugguuccucggaacuaaacucgugguuccugugguucacaccugaccuccugagcagaaaagaaaaaagaaggcggcucggaggagcgcuucagggauccccggggaaaccuggag

SEQ ID NO: 13 - RNA, 406 nt, Artificial Sequence
  Synthetic Construct
caaacacacgcucggacgcauauuacacauguucauacacuuaauacucgcuguuuugaauugauguuuuaggaauauauauguagaguagaaagguagcagcaugguucacaggucgugauaugauucaauuagcuuccgacucauucauccaaauaccgagucgccaaaauucaaacuagacucguuaaaugaaugaaugaugcgguagacaaauuggaucauugauucucuuugauccaugcagcuaccuuuccacucucucuuuuguauuccaauuuucuugauuaaucuuuccugcacaaaaacaugcuugauccacuaagugacauauaugcugccuucguauauauaguucugguaaaauuaacauuuuggguuuaucuuuauuuaaggcaucgccaug

SEQ ID NO: 14 - RNA, 473 nt, Artificial Sequence
  Synthetic Construct
gguguuuagcccuuggaaccgcaguugguuccucggaacuaaacucgugguuccugugguucacacccaaacacacgcucggacgcauauuacacauguucauacacuuaauacucgcuguuuugaauugauguuuuaggaauauauauguagaguagaaagguagcagcaugguucacaggucgugauaugauucaauuagcuuccgacucauucauccaaauaccgagucgccaaaauucaaacuagacucguuaaaugaaugaaugaugcgguagacaaauuggaucauugauucucuuugauccaugcagcuaccuuuccacucucucuuuuguauuccaauuuucuugauuaaucuuuccugcacaaaaacaugcuugauccacuaagugacauauaugcugccuucguauauauaguucugguaaaauuaacauuuuggguuuaucuuuauuuaaggcaucgccaug

SEQ ID NO: 15 - RNA, 624 nt, Artificial Sequence
  Synthetic Construct
cuucggcuacuacccgguggaaacaacugaagcucccgagaaccgcuuuuucucuaucuuacuugcuucggggcgaggguguuuagcccuuggaaccgcaguugguuccucggaacuaaacucgugguuccugugguucacaccugaccuccugagcagaaaagaaaaaagaaggcggcucggaggagcgcuucagggauccccggggaaaccuggagcaaacacacgcucggacgcauauuacacauguucauacacuuaauacucgcuguuuugaauugauguuuuaggaauauauauguagaguagaaagguagcagcaugguucacaggucgugauaugauucaauuagcuuccgacucauucauccaaauaccgagucgccaaaauucaaacuagacucguuaaaugaaugaaugaugcgguagacaaauuggaucauugauucucuuugauccaugcagcuaccuuuccacucucucuuuuguauuccaauuuucuugauuaaucuuuccugcacaaaaacaugcuugauccacuaagugacauauaugcugccuucguauauauaguucugguaaaauuaacauuuuggguuuaucuuuauuuaaggcaucgccaug

SEQ ID NO: 16 - RNA, 289 nt, Artificial Sequence
  Synthetic Construct
gugguacaaggcaugggaaauguuuauauuggugugggcaauauacuccucauuguucacucccauggaguuugguuucuuccgcggucugccugagagacucuuuguacuugacauuguuggucagaucgcguucuuggucgauauuguucuucaguucuuuguugccuaucgcgauacucagaccuaccggacugucuacaaaccaacacguauugcuuuccgguacuugaagucgcauuuucucauggauuucaucgguugcuucccuugggaucuuauuuauaag

SEQ ID NO: 17 - RNA, 356 nt, Artificial Sequence
  Synthetic Construct
gugguacaaggcaugggaaauguuuauauuggugugggcaauauacuccucauuguucacucccauggaguuugguuucuuccgcggucugccugagagacucuuuguacuugacauuguuggucagaucgcguucuuggucgauauuguucuucaguucuuuguugccuaucgcgauacucagaccuaccggacugucuacaaaccaacacguauugcuuuccgguacuugaagucgcauuuucucauggauuucaucgguugcuucccuugggaucuuauuuauaaggguguuuagcccuuggaaccgcaguugguuccucggaacuaaacucgugguuccugugguucacacc

SEQ ID NO: 18 - RNA, 63 nt, Artificial Sequence
  Synthetic Construct
gacaggaguaauucccgccgaaacaggguuuucacccuuccuuucuucggguguccuuccucg

SEQ ID NO: 19 - RNA, 154 nt, Artificial Sequence
  Synthetic Construct
ggacccacauacucugaugauccgagacggucggguccagauauucguaucugucgaguagagugugggcucggaucauucauggcaagagacggucggguccagauauucguaucugucgaguagagugugggcucuugccauguguaugugg

SEQ ID NO: 20 - RNA, 526 nt, Artificial Sequence
  Synthetic Construct
gccuugauuugagcaccaaggacaccaaguguggacuggaauuguggcacgcacaacuguuaaaauggagaccgccacuauuaccaacguccuggguguaugagacuacuaggcucugccagcccaggucuauaagcauagacagcucaucucacacccgagccuaguaaguaccguucucugccagcccaggucuauaagcauagacagcucaucucacacccgagaacgguacacauacacccgcgaagucccuaggggccccuuuggaccucgcuugaccguuuuuuccugccaccccucacgggucgccggcuguccucauuaagggcggcuuugucccaaaagugggaaggaaagaagcccacaggaaggagcgcgggcguccugguggggagcgggggaaacgcgacagcgaagccgaugaugggccaccuuuguugacuucgagggcucuuggcgaaaaagagauagaaugaacgaagccccgcucccacaaaucgggaaccuuggcgucaaccaagga

SEQ ID NO: 21 - RNA, 40 nt, Artificial Sequence
  Synthetic Construct
gacucuuaacuauagaucuuaaucucguuugcaacguuac

SEQ ID NO: 22 - RNA, 528 nt, Artificial Sequence
  Synthetic Construct
ggaugauggagcccacacucuacucgacaggaucaucagaguauguggguugacccacauacacauggcaagagcccacacucuacucgacagaugaugcggauccugcaaccauuaucaccgccagagguaaaauugucaacacgcacgguguuagcucacauaugcggugugaaauaccgcacagaugcguaaggagaaaauaccgcaucaggcgccauucgccauucaggcugcgcaacuguugggaagggcgaucggugcgggccucuucgcuauuacgccagcuggcgaaagggggaugugcugcaaggcgauuaaguuggguaacgccaggguuuucccagucacgacguuguaaaacgacggccagugaauucgagcucgguacccggggauccucuagagucgaccugcaggcaugcaagcuuaaaaaaaagcccgccuuucgggggcuuugauccgcaucaucuggaccagaccgucucuugccaugaaugauccuggacccgaccgucuccaucaucc

SEQ ID NO: 23 - RNA, 631 nt, Artificial Sequence
  Synthetic Construct
guuuuuuggggaguucugggcaaaucuccgggguuccccaauacgauccuacuaccucgggugugagaugagcuguccuaguagucucauacacccaacuggguguauguguaccguucucgggugugagaugagcugucuacuacgccuaggacguugguaauaguggcggucuccauuuuaacaguugugcgugccacaaucgaguguauacgccacacuuuauggcgugucuacgcauuccucuuuuauggcguaguccgcgguaagcgguaaguccgacgcguugacaacccuucccgcuagccacgcccggagaagcgauaaugcggucgaccgcuuucccccuacacgacguuccgcuaauucaacccauugcggucccaaaagggucagugcugcaacauuuugcugccggucacuuaagcucgagccaugggccccuaggagaucucagcuggacguccguacguucgaauuuuuuuucgggcggaaagcccccgaaacuaggcguaguagaccuggucuggcagagaacgguacuuacuaggaccugggcuggcagagguaguaggacguugguaauaguggcggucuccauuuuaacaguugugcgugccacaauacuuuu

SEQ ID NO: 24 - skipped

SEQ ID NO: 25 - RNA, 27 nt, Artificial Sequence
  Synthetic Construct
gcagaaaagaaaaaagaaggcggcucg

SEQ ID NO: 26 - RNA, 98 nt, Artificial Sequence
  Synthetic Construct
gacgcaacugaaugaaauggugaaggacggguccagguguggcugcuucggcagugcagcuuguugaguagagugugagcuccguaacuagucgcguc

SEQ ID NO: 27 - RNA, 383 nt, Artificial Sequence
  Synthetic Construct
cggaacuaaacucgugguuccugugguucacaccugaccuccugaggugaaggacggguccaguaguucgcuacuguugaguagagugugagcuccgaggagcgcuucagggauccccggggaaaccuggagcgaacuggcaaaaaaggacgguggggagugcccagcggccgacaggaguaauucccgccgaaacaggguuuucacccuuccuuucuucggguguccuuccucgcgcccgcaggaccaccccucgcccccuuugcgcugucgcuucggcuacuacccgguggaaacaacugaagcucccgagaaccgcuuuuucucuaucuuacuugcuucggggcgaggguguuuagcccuuggaaccgcaguugguuccu

SEQ ID NO: 28 - RNA, 333 nt, Artificial Sequence
  Synthetic Construct
ggguggugugugccaccccugaugagaccgaaaggucgaaaugggguuucgccaugggucgggacuuuaaauucggaggauucguccuuuaaacguuccuccaagagucccuuccccaaacccuuacuuuguaagugugguucggcgaauguaccguuucguccuuucggacucaucagggaaaguacacacuuuccgacgguggguucgucgacaccucucccccucccagguacuauccccuuucaaggauguguucccuaggaggguggguguaccucuuuuggauugcuccggccuuccaggagagauagaggacgaccucuccccaua

SEQ ID NO: 29 - RNA, 484 nt, Artificial Sequence
  Synthetic Construct
ccccauaggguggugugugccaccccugaugagaccgaaaggucgaaaugggguuucgccaugggucgggacuuuaaauucggaggauucguccuuuaaacguuccuccaagagucccuuccccaaacccuuacuuuguaagugugguucggcgaauguaccguuucguccuuucggacucaucagggaaaguacacacuuuccgacgguggguucgucgacaccucucccccucccagguacuauccccuugacgcaacugaaugaaauggugaaggacggguccagguguggcugcuucggcagugcagcuuguugaguagagugugagcuccguaacuagucgcgucucaaggauguguucccuaggaggguggguguaccucuuuuggauugcuccggccuuccaggagagauagaggacgaccucuccccauaggguggugugugccaccccugaugagaccgaaaggucgaaaugggg

SEQ ID NO: 30 - skipped

SEQ ID NO: 31 - RNA, 406 nt, Artificial Sequence; identical to SEQ ID NO: 13
  Synthetic Construct

SEQ ID NO: 32 - RNA, 469 nt, Artificial Sequence
  Synthetic Construct
gacaggaguaauucccgccgaaacaggguuuucacccuuccuuucuucggguguccuuccucgcaaacacacgcucggacgcauauuacacauguucauacacuuaauacucgcuguuuugaauugauguuuuaggaauauauauguagaguagaaagguagcagcaugguucacaggucgugauaugauucaauuagcuuccgacucauucauccaaauaccgagucgccaaaauucaaacuagacucguuaaaugaaugaaugaugcgguagacaaauuggaucauugauucucuuugauccaugcagcuaccuuuccacucucucuuuuguauuccaauuuucuugauuaaucuuuccugcacaaaaacaugcuugauccacuaagugacauauaugcugccuucguauauauaguucugguaaaauuaacauuuuggguuuaucuuuauuuaaggcaucgccaug

SEQ ID NO: 33 - RNA, 87 nt, Artificial Sequence
  Synthetic Construct
cggaacuaaacucgugguuccugugguucacaccugaccucgcuucggggcgaggguguuuagcccuuggaaccgcaguugguuccu

SEQ ID NO: 34 - RNA, 185 nt, Artificial Sequence
  Synthetic Construct
cggaacuaaacucgugguuccugugguucacaccugaccucgcuucggggcgaggguguuuagcccuuggaaccgcaguugguuccugacgcaacugaaugaaauggugaaggacggguccagguguggcugcuucggcagugcagcuuguugaguagagugugagcuccguaacuagucgcguc

SEQ ID NO: 35 - RNA, 1206 nt, Artificial Sequence
  Synthetic Construct
cccuuaucggcuuuuuguuuuuuguuuuuuuuguuuuuuuuuugguuuuuuuguuuugugugccuugauuugagcaccaaggacaccaaguguggacuggaggacuugcaaugaccggcuucggcgaaccuuauuccggccacacgcaaacagauauacaauaaaaggugguauaacggcagaaaaccguuacacucccgggccuuuggaccgggacagaagaacugcucguaaggauccccagaaaggggagagcgguuuccuuacguuccagacaacuuacagcacuuccuucgucaaggagaccuucgaagaacuucuguuuguugcagacaucgcugggaaacguccgucgccuugggggguggaccgcuguccacggagacgccgguuuucggugcacauauucuauguggacguuuccgccguguuggggucacggugcaacacucaaccuaucaacaccuuucucaguuuaccgagaggaguucgcauaaguuguuccccgacuuccuacgggucuuccaugggguaacauacccuagacuagaccccggagccacguguacgaaauguacacaaaucagcuccaauuuuuugcagauccggggggcuuggugccccugcaccaaaaggaaacuuuuugugcuacuauuaucggugguaccagaagugugagcuucuaaagcaaccccugaccgcugucugucggccgauguuggaccugguucaggaacuugucccuccacacaggucaaacaaagucuuagagccccacaggcauugaggcuagguuuccuaacaggacucgccacuuuuacccgacuucuagcuguagguacaguaguagggcauacuuccagacucgccgcugguuuacccggucuagcuuuuuuaaaaauuccaccacaugggacaccuacuaguagugaaauuccacuaggacgugauaccgugugaccauuagcugccccaaugcggcuuguacuagcugauaaagccugccggcauacuuccguagcggcacaagcugccguuuuucuagugacauugucccugggacaccuugccguuguuuuaauagcugcucgcggacuaguuggggcugccgagggacgacaaggcucauugguaguugccucacuggccgaccgccgacacgcuugcguaagaccgcauuuuuuuuguuuuuuguuuugccgauaa

SEQ ID NO: 36 - RNA, 359 nt, Artificial Sequence
  Synthetic Construct
cggaacuaaacucgugguuccugugguucacaccugaccuccugagcagaaaagaaaaaagaaggcggcucggaggagcgcuucagggauccccggggaaaccuggagcgaacuggcaaaaaaggacgguggggagugcccagcggccgacaggaguaauucccgccgaaacaggguuuucacccuuccuuucuucggguguccuuccucgcgcccgcaggaccaccccucgcccccuuugcgcugucgcuucggcuacuacccgguggaaacaacugaagcucccgagaaccgcuuuuucucuaucuuacuugcuucggggcgaggguguuuagcccuuggaaccgcaguugguuccu

SEQ ID NO: 37 - RNA, 464 nt, Artificial Sequence
  Synthetic Construct
gagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaau

SEQ ID NO: 38 - RNA, 147 nt, Artificial Sequence
  Synthetic Construct
agcacaacauuucaaccagaaacacuagccgaagcaaauccauuccacaagcaccuggugggaucaucucaucaucagaaaccaagagagagauuccguguccgcuuguuguaguagauugugaggacugaggaccgagaagcagcc

SEQ ID NO: 39 - RNA, 516 nt, Artificial Sequence
  Synthetic Construct
auggucuucacacucgaagauuucguuggggacuggcgucagacugcuggguauaaucuagaucagguucuggaacaaggugguguuagcucccuguuucagaaucugggugucucagugacuccaauucagaggaucgugcuuagcggggaaaauggguuaaagaucgauauucauguaauaauccccuaugagggacuuuccggggaucagaugggacaaaucgagaagaucuucaaggucguuuauccaguagacgaucaccauuuuaagguuauacuccauuacggcacccuagugauugacgggguaaccccaaacaugauagauuauuucgggaggcccuacgaagguaucgcuguauuugacggaaagaagauaacagucaccggcacacuauggaauggaaacaagauaauagacgagagacuaaucaauccagauggcagucucuuauuucguguaaccauaaauggagugaccggcuggcggcugugcgaacgcauucuggcguaa

SEQ ID NO: 40 - RNA, 801 nt, Artificial Sequence
  Synthetic Construct
gggaauagccgaaaaacaaaaaacaaaaaaaacaaaaaaaaaaccaaaaaaacaaaacacacggaacuaaacucgugguuccugugguucacaccugaccuccugaagcacaacauuucaaccagaaacacuagccgaagcaaauccauuccacaagcaccuggugggaucaucucaucaucagaaaccaagagagagauuccguguccgcuuguuguaguagauugugaggacugaggaccgagaagcagccgccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaaaaaacaaaaaacaaaacggcuauu

SEQ ID NO: 41 - RNA, 87 nt, Artificial Sequence; identical to SEQ ID NO: 33
  Synthetic Construct

SEQ ID NO: 42 - RNA, 185 nt, Artificial Sequence; identical to SEQ ID NO: 34
  Synthetic Construct

SEQ ID NO: 43 - RNA, 185 nt, Artificial Sequence; identical to SEQ ID NO: 34
  Synthetic Construct

SEQ ID NO: 44 - skipped

SEQ ID NO: 45 - RNA, 47 nt, Artificial Sequence
  Synthetic Construct
cgggaucuuuuccuugugguuccugugguucacaccugaccuccugc

SEQ ID NO: 46 - RNA, 232 nt, Artificial Sequence
  Synthetic Construct
cggaacuaaacucgugguuccugugguucacaccugaccucgcuucggggcgaggguguuuagcccuuggaaccgcaguugguuccucgggaucuuuuccuugugguuccugugguucacaccugaccuccugcgacgcaacugaaugaaauggugaaggacggguccagguguggcugcuucggcagugcagcuuguugaguagagugugagcuccguaacuagucgcguc

SEQ ID NO: 47 - RNA, 631 nt, Artificial Sequence; identical to SEQ ID NO: 23
  Synthetic Construct

SEQ ID NO: 48 - RNA, 297 nt, Artificial Sequence
  Synthetic Construct
cggaacuaaacucgugguuccugugguucacaccugaccuccugagcagaaaagaaaaaagaaggcggcucggaggagcgcuucagggauccccggggaaaccuggagcgaacuggcaaggugaaggacggguccaguaguucgcuacuguugaguagagugugagcuccuugcgcugucgcuucggcuacuacccgguggaaacaacugaagcucccgagaaccgcuuuuucucuaucuuacuugcuucggggcgaggguguuuagcccuuggaaccgcaguugguuccuugaaaa

SEQ ID NO: 49 - DNA, 2462 bp, Artificial Sequence
  Synthetic Construct
gaactcgagcagctgaagcttgcatgcctgcaggtcgactctagaggatccccgggtaccgagctcgaattcatcgatgatatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 50 - RNA, 333 nt, Elaviroid; identical to SEQ ID NO: 28
  AJ536613.1 Eggplant latent viroid complete genome, isolate 2

SEQ ID NO: 51 - RNA, 359 nt, Pospiviroid; identical to SEQ ID NO: 8
  U23058.1 Potato spindle tuber viroid (PSTVd) strain RG 1, complete genome

SEQ ID NO: 52 - RNA, 475 nt, Pospiviroid
  AY671952.1 Citrus exocortis viroid isolate D-104, complete genome
cgggaacuuucuugagguuccuguggugcucaccugacccugcaggcagaaaaagaaaaaagaggcggcgggggaagaaguccuucagggauccccggggaaaccuggaggaagucgaggucgggggggaucagcugcuucggucgccgcggaucacuggcgaccagcggagaaacaggagcucagcugcuucggucgccgcggaucacuggcguccagcggagaaacaggagcucgucuccuuccuuucgcugcuggcuccacauccgaucgucgcugaagcgucugucuccuuccuuucgcugcuggcuccacauccgaucgucgcugaagcgccuugcccccucgcccggagcuucucucuggagacuacccgguggaaacaacugaagcuucaaccccaaaccgcuuuucuuauaucuuuacugcucuccgggcgagggugaaagcccuuggaacccuagaaugggucccu

SEQ ID NO: 53 - RNA, 456 nt, Pospiviroid
  DQ076250.1 Columnea latent viroid clone PC-2-Pa54 plus 88, complete genome
cggaacuaaacucgugguuccugugguucacaccugacccugcagccaugcaaagaaaaaagaacgggaggaagagcgcaagagcggucucaggagccccggggcaacucagaccgagcggggaucgcggaccgagggcggaagccugcuucagacaggaguaaucccaguagaaacaggaguaaucccugcugaaacaugguuuucacccuuccuuuccacgcagggguaaucccaguaaaacauggguuuucacccuuccuuucggguuuucacccuuccuuucuucggguuuccuuccucugcuugcggccucgcccggagucuucgaauccagcgcagguucugacgcgaccgguggcacgccgagucgcucacugccucaaccuccuuuuccucuaucuagcuuggucuccgggcgaggguguuuagcccuuggaaccgcaguugguuccu

SEQ ID NO: 54 - RNA, 448 nt, Pospiviroid
  AY671954.1 Citrus exocortis viroid isolate D-76, complete genome
cgggaacuuucuugagguuccuguggugcucaccugacccugcaggcagaaaaagaaaaaagaggcggcgggggaagaaguccuucagggauccccggggaaaccuggaggaagucgaggucgggggggacagcugcuucggucgccgcggaucacuggcguccagcggagagcugcuucggucgccgcggaucacuggcguccagcggagaaacaggagcucgucuccuuccuuucgcugcuggcuccacauccgaucgucgcugaagcgcccgcugcuggcuccacauccgaucgucgcugaagcgccacgcccccucgcccggagcuucucucuggagacuacccgguggaaacaacugaagcuucaaccccaaaccgcuuuucuuauaucuuaacugcucuccgggcgagggugaaagcccuuggaacccuagauugggucccu

SEQ ID NO: 55 - RNA, 436 nt, Pelamoviroid
  MK947213.1 Apple hammerhead viroid isolate Gala, complete genome
ggaggcuauuagccuuccugaugaguccguuccaaggacgaaaccgucucuccugacucccgguucugguuagguuuggguaucccuaccaguuacuuccgguaacuaggaguuuguagugacguaaguuucguccuaaaaggacucaucagguagccuaauagacuacgacuuacgcgguaggucgugaacaagcccggaguacauucccaauuggaacuagggaaccccuccggucuuguccaaccucuguuucggcagaggauaccgugcuccacgguuggaaggccgugggcccgagagaagucgcucucucucgcccuguacauccucucaggcauagagaugaccauuugccuagggugagucuuuaaguagacacagccuaucuauggugauguauuggggcacggguuuugacacccccgugacgguc

SEQ ID NO: 56 - RNA, 434 nt, Pelamoviroid
  MK188695.1 Apple hammerhead viroid isolate SD17_9-2, complete genome
ggaggcuauuagccuuccugaugaguccguuccaaggaagaaaccgucucuccugacucccggugucgaugugguacguguaucccacucggcuacuuccgguaacuaggaguuugcagugacguaaguuucguccuaaaaggacucaucagguagccuaauagacuacgacuuacgcgguaggucgugaauacgcccggaguacauucccaauggaacuagggaaccccuccggucguauccaaccucuguuucggcagaggauaccgugcuccacagcuggaaggcugugggcccgagagaagcgcucucucucgcccuucuacucccccucggcauagagauguccauaggccaggggugagauccaacgggaucacagcugaucuaagguggguaguaggggcacggguuuuaucacccccaugacgguc

SEQ ID NO: 57 - RNA, 434 nt, Pelamoviroid
  MK188693.1 Apple hammerhead viroid isolate SD17_4-1, complete genome
ggaggcuauuagccuuccugaugaguccguuccaaggacgaaaccgucuacccagacucccgguucugguuggguuaguauaccaacuaguuacuuccgguaacuugggguuuguguugacguaaguuucguccuaaaagggcucaucagguagccuaauagacuacgacuuacgcgguaggucgugaauaaggccggaguacauucccuauggaacuaagggaaccccuccggucuuauccaaccucuguuucggcagaggauaccgugcucccaagcuguaaggcuuggggcccgagagaagucgcucucucucgcccuguccauccucucaggcauagagaugaccauuugccuagggugagucuuuaaguagacacagccuaucuauggcgauggauuggggcacggguuuuaacacccccaugacgguc

SEQ ID NO: 58 - RNA, 434 nt, Pelamoviroid
  MH643727.1 Apple hammerhead viroid strain AHVd-IT_MRG_16, complete sequence
ggaggcuauuagccuuccugaugaguccguuccaaggacgaaaccgucucuccagacucccgguucuguggugguuuguguaucccacacaguuacuuccgguaacuuggaguuuguagcgacauaaguuucguccuaaaaggacucaucagguagccuaauagacuacgacuuaugcgguaggucgugaauacgcccggaguacaucuccaauggaacuaggagaccccuccggucguauccaaccucuguuucggcagaggauaccgugcuccacagcuggaaggcugugggcccgagagaagcgcucucucucgcccuucuacucccccucggcauugagauguccauaggccaggggugagauccaacgggaucacagcugaucuaagguggguaguaggggcacggguuuuaucacccccgugacgguc

SEQ ID NO: 59 - RNA, 434 nt, Pelamoviroid
  MH643711.1 Apple hammerhead viroid strain AHVd-IT_Ag_16, complete sequence
gaaggcuauuagccuuccugaugaguccguuccaaggacgaaaccguuucuccagacucccgguucugaggugguuuguguaucccacucaguuacuuccgguaacuuggaguuuguaguaacguaaguuucguccuaaaaggacucaucagguagccuaauagacuacgacuuacgcgguaggucgugaauaaggccggaguacauucucuauggaacuaagggaaccccuccggucuuauccaaccucuguuucggcagaggauaccgugcuccacagcuguaaggcugugggcccgagagaagcgcucucucucgcccuucugcucccccucggcauugagauguccauaggccaggggugagauccaacgggaucacagcugaucuaaggugggcaggagggguauggguuaugucgacacccaacgguc

SEQ ID NO: 60 - RNA, 434 nt, Pelamoviroid
  MH643700.1 Apple hammerhead viroid strain AHVd-IT_Ag_05, complete sequence
ggaggcuauuagccuuccugaugaguccguuccaaggacgaaaccgucuucccagacucccggugucgaggugguuuguguaucccacucggcuacuuccgguaacuugggguuugugcugacauaaguuucguccuaaaaggacucaucagguagccuaauagacuacgacuuaugcgguaggucgugaauacgcccggaguacaucuccaauggcacuaggagaccccuccggucguauccaaccucuguuucggcagaggauaccguacuccacagcuguaaggcugugggcccgagagaagcgcucucucucgcccuucugcucccccucggcauagagauguccauaggccaggggugagauccaacgggaucacagcugaucuaaggugggcagcaggggcacggguuuugagacccccgugacgguc

SEQ ID NO: 61 - RNA, 433 nt, Pelamoviroid
  MK188705.1 Apple hammerhead viroid isolate AP_SD15_APP1_1, complete genome
ggaggcuauuagccuuccugaugaguccguuccaaggacgaaaccgucucuccagacucccgguucugguuggguuaguuauaccaacuaguuacuuccgguaacuuggaguuuguagcgacauaaguuucguccuaaaaggacuuaucagguagccuaauagacuacgacuuaugcgguaggucgugaauaaggccggaguacauucccaauggaacuagggaaccccuccggucuuauccaaccucuuuuucggaagaggauaccguacucccaagcuguaaggcuuggggcccgagagaagucgcucucucucgcccuauacauccucucaggcauagagaugaccauuugccuagggugagucuuuaaguagacacagccuaucuauggugauguacuggggcauggguuuagaccacccaugacgguc

SEQ ID NO: 62 - RNA, 433 nt, Pelamoviroid
  MK188703.1 Apple hammerhead viroid isolate SD18_16-1, complete genome
ggaggcuauuagccuuccugaugaguccguuccaaggaugaaaccgucucuccuaauucccaguuuccauagguuaauuauccucauggauuacuucugguaaccaggaguuuguagcgacguaaguuucguccuaaaaggacucaucagguagccuaauagacuacgacuuacgcggugggucgugaauaagcccggaguacauucccaauggaacuagggaaccccuccggucuuauccaaccucuguuucggcagaggacaccgugcuccacaguuguaagacugugggcccgagagaagucgcucucucucgccccauacauccucucaggcauagagaugaccauuugccuagggugagucuuuaaguagacacagccuaucuauggcgauguaucggggcacggguuuuaacacccccaugacgguc

SEQ ID NO: 63 - RNA, 433 nt, Pelamoviroid
  MK188702.1 Apple hammerhead viroid isolate SD18_6-5, complete genome
ggaggcuauuagccuuccugaugaguccguuccaaggacaaaaccgucucuccagacucccgguucugguuggguuaguuauaccaacuaguuacuuccgguaacuuggaguuuguacagacauaaguuucguccuaaaaggacucaucagguagccuauuagacuacgacuuaugcgguaggucgugaauaagaacggaguacaucuccaauggaacuaggagaccccuccguucuuauccaaccucuguuucggcagaggauaccguacucccuagcuguaaggcuaggggcccgagagaagucgcucucucucgcccuuuccauccucucaggcauagagaugaccauuugccuagggugagucuuuaaguagacacagccuaucuauggcgguggauaggggcacggguuuaacaccaccaugacgguc

SEQ ID NO: 64 - RNA, 433 nt, Pelamoviroid
  MK188692.1 Apple hammerhead viroid isolate SD17_3-3, complete genome
ggaggcuauuagccuuccugaugaguccguuccaaggacgaaaccgucucuccugacucccgguucugguuggggacuguaccaaccaguuacuuccgguaacuaggaguuuguagugacguaaguuucguccuaagaggacuaauuagguagccuaauagacuacgacuuacguggugggucgugaacaagcccggaguacauucccuauuggaacuaagggaaccccuccggucuuguccaaccucuguuucggcagaggacaccgcgcuccauagcuguaaggcuaugggcccgagagaagucgcucucucucgcccuucacaaccuucucggcauugagaugaccauaggccagaagugagucuuuaaguagacacagcugaucuaaggcguuguguaggggcacggguuuucaacccccaugacgguc

SEQ ID NO: 65 - RNA, 415 nt, Pospiviroid
  AY671955.1 Citrus exocortis viroid isolate D-43, complete genome
cgggaacuuucuugagguuccuguggugcucaccugacccugcaggcaguaaaagaaaaaagaggcggcgggggaagaaguccuucagggauccccggggaaaccuggaggaagucgaggucgggggggacuagcuguucggucgccgcggaucacuggcguccagcggagaaacaggagcucgucuccuuccuuucgcugcuggcuccacauccgaucgucgcugagucuccuuccuuucgcugcuggcuccacauccgaucgucgcugaagcgccacgcccccucgcccggagcuucucucuggagacuacccgguggaaacaacugaagcuucaaccccaaaccgcuuuucuuauaucuucucugcucuccgggcgagggugaaagcccuuggaacccuagauugggucccu

SEQ ID NO: 66 - RNA, 413 nt, Pospiviroid
  AY671956.1 Citrus exocortis viroid isolate D-40, complete genome
cggaacuuuucuugaagguuccuguggugcucaccuugacccugcaggcaguaaaagaaaaaagaggcggcgggggaagaaguccuucagggauccccggggaaaccuggagaagucgaggucgggggggacagcugcuucggucgccgcggaucacuggcguccagcggagagcugcuucggucgccgcggaucacuggcguccagcggagaaacaggagcucgucuccuuccuuucgcugcuggcuccacauccgaucgucgcugaagcgccucgcccccucgcccggagcuucucucuggagacuacccgguggaaacaacugaagcuucaaccccaaaccgcuuuucuuauaucuucucugcucuccgggcgagggugaaagcccuuggaacccuagauuggguccccu

SEQ ID NO: 67 - RNA, 410 nt, Pelamoviroid
  MH049334.1 Apple hammerhead viroid isolate AHVd_17_New_Zealand, partial genome
ugaugaguccguuccaaggacgaaaccgucucuccugacucccgguucaguggugguuuguguaucccacacuguuacuuccgguaacuaggaguuuguagcgacauaaguuucguccuaaaaggacucaucagguagccuaauagacuacgacuuaugcgguaggucgugaauacgcccggaguacauccucaauggaacuagaggaccccuccggucguauccaaccucuguuucggcagaggauaccguacuccauagcuguaaggcuaugggcccgagagaagucgcucucucucgcccuucacaucccucucggcauagagaugaccauaggccaggggugaguccuuaaguggacacagcugaucuaaggcgguguguaggggcacggguuuucaacccccaug

SEQ ID NO: 68 - RNA, 405 nt, Pospiviroid
  LQ907271.1 Sequence 53 from Patent WO2018179834
ucuagacggaacuaaacucgugguuccugugguucacaccugaccuccugagcagaaaagaaaaaagaaggcggcucggaggagcgcuucagggauucccggggaaaccuggagcgaacuggcaacaaggacgguggggagugcccagcggccgacaggaguaauucccgccgaaacaggguuuucacccuuccuuucuucggguguccuuccucgcgcccgcaggaccaccccucgcccccuuugcgcugucgcuucggcuacuacccgguggaaacaacugaagcucccgagaaccgcuuuuucucuaucuuacuugcuucggggcgaggguguuuagcccuuggaaccgcaguugguuccuggauccggggaauuguuauuuguuuauaauuuucccucgag

SEQ ID NO: 69 - RNA, 401 nt, Pelamoviroid
  AJ247113.1 Chrysanthemum chlorotic mottle viroid RNA, variant CMNS 2
ggcaccugaugucgguguccugacgaagauccaugacaggaucgaaaccucuuccaguuucggcuugugcgggaguaaagcaauuugcucucuccacagccucgucaggaaacccacuucaggucucgacuggaaggucguuaaacuuccccucuaagcggaguagagguaaauaccuccgucccaaccccgggaggaaagggguuggggcccggaacagauccaguuccgguccuuuggaguccauuucucucguuggauauucuccucggagaagggagauggggccagucccagucgguucgcucucguagucacagccacuggggaaccuaggcagauggcuggacggagucuuaguccacuccagaggaccuuggguuucuaacccccaagagguc

SEQ ID NO: 70 - RNA, 400 nt, Pelamoviroid
  KT033024.1 Chrysanthemum chlorotic mottle viroid isolate Ford-19, complete genome
cucuuccaguuucggcuugugcgggaguaaagcaauuugcucucuccacagccucaucaggaaacccacuucaggucucgacuggaaggucguaaaacuuccccucuaagcggaguagagguaaauaccuccgucccaccccgggaggaaagggguggggacccggagcagauccggcuccgguccuuuggaguccauuucucucguuggauauucuccucggggaagggagauggggccagucccagucgguucgcucucguagucacagccgcuggggaaccuaggcagaaggcuggacggagucuuaguccacuccaaaggaccuuggguuuuaaacccccaagaggucggcaccugaagucgguguccugaugaagauccaugacaggaucgaaac

SEQ ID NO: 71 - RNA, 400 nt, Pelamoviroid
  DQ402041.1 Chrysanthemum chlorotic mottle viroid RNA, complete genome
ggcaccugacgucgguguccugacgaagauccaugacaggaucgaaaccucuuccaguuucggcuugugcgggaguaaaucgaaagaucucuccacagccucgucaggaaacccacuucaggucucgacuggaaggucguaaaacuuccucucuaagcggaguagagguaaauaccuccgucccaucccgggaggaaagggauggggacccggaacagauccaguuccgguccuuuggaguccauuuuucucguuggauauucuccucggagaagggaaauggggccagucccagucgguucgcucucguagucacagccgcuggggaaccuaggcagaaggcuggacggagucuuaguccacuccaaaggaccuuggguuuagaaacccccaagagguc

SEQ ID NO: 72 - RNA, 399 nt, Pelamoviroid
  LC089753.1 Chrysanthemum chlorotic mottle viroid genomic RNA, complete genome, strain: K6pop
cuucaggucucgacuggaaggucguuaacuuccccucuaagcggaguagagguaaauaccuccguuccaaccccgggaggaaagggguugggacccggagcagauccagcuccgguccuuuggaguccauuucucucguuggauauucuccucggagaagggagauggggccagucccagucgguucgcucucguagucacagccgcuggggaaccuaggcagaaggcuggacggagucuuaguccacuccaaaggaccuuggguuucuaacccccaagaggucggcaccugaugucgguguccugacgaagauccaugacaggaucgaaaccucuuccaguuucggcuugugcgggaguaaagcuuucgcucucuccacagccucgucaggaaaccca

SEQ ID NO: 73 - RNA, 399 nt, Pelamoviroid
  AJ878089.1 Chrysanthemum chlorotic mottle viroid, clone CM298 VR
ggcaccugacgucgguguccugaugaagauccaugacaggaucgaaaccucuuccaguuucggcuugugcgggaguaaagcuuucgcucucuccacagccucaucaggaaacccacuucaggucucgacuggagggucguuaaacuuccccucuaagcggaguagagguaaauaccuccgucccaccccgggaggaaagggguggggacccggaacagcucuaguuccgguccuuuggaguccguuucuaccguuggagauuaccuccggguaagggagacggggccagucccagucgguucgcucucguagucacagccgcuggggaaccuaggcagauggcuggacggagucuuaguccacuccaaaggaccauggguuuuaaacccccaugagguc

SEQ ID NO: 74 - RNA, 396 nt, Apscaviroid
  NC_010308.1 Persimmon viroid, complete genome
cggacuaaaacaugugguuccuguggugacaccucucgaucccuuaccugcaaaagaaaaggaaaaagaggcgcgaaggacgaaagacucgucgucgacgaaggguucuuucguucgcaaggcucccugccgagcucggggcuggagcuugggacugcucauagagagcaggucgccagccugggguugaaaagaccuccgggucuuucuuccuuccagcagugcuuccuugccacucggagaacccaccgcucucccccgccgcucugguaaggccccugcagcgaagaucccgcuagucgagcggacuugucgaucaaccaacgcgcuuuucuuuuauucccucuugcgcggcggggcgagaguguuuugccuaaggaccgcuguuuggguccu

SEQ ID NO: 75 - RNA, 396 nt, Pospiviroid
  FJ751933.1 Citrus exocortis Yucatan viroid isolate 15, complete genome
uauccccggggaaaccuggaggaagguggggaugacgucaaguccgcauggcccuuaugggcugggccacacacgugcuacaauggcaauuacaaugggaagcaaggcuguaaggcggagcgaauccggaaagauugccucaguucggauuguucucugcaacucgggaacaugaaguuggaaucgcuaguaaucgcggaucagcaugccgcggugaauauguacccgggcccuguacacaccgcccgucacacccugggaauugguuucgcccgaagcaucggaccaaugaucacccaugacuucuguguaccacuagugccacaaaggcuuuugguggucuuauuggcgcauaccacgguggggucuucgaucuggggaagaaguccuucaggg

SEQ ID NO: 76 - RNA, 389 nt, Apscaviroid
  KP010010.1 Grapevine yellow speckle viroid 1 clone Poochidpha-3, complete genome
ucuccggaucuucuugcuugugguuccugugguuucaccucggaaggccgccgcggaccugcaaagaagaagauaggggcagagggggagugagccucgggcagagggggagugagccucgucgucgacgaaggggugcacuccgagugccugagcuggucgacguccagcucccucgggaccugcugcucugggcggaagagucuucugacacuucuagccuauucagcaucgcguccuugaggccuggcguaacgcggcucuugccaccaggaugccuccgcuagucgagcggacuuggucucuuuccgcccaaagcccuuuuucuuucaucugagcuuguuccaacgcgccccuugagugcaauccccggaacccccgcuaagagg

SEQ ID NO: 77 - RNA, 386 nt, Pospiviroid
  KC290928.1 Citrus exocortis viroid strain CMC-D, complete genome
cgggaucuuucuugagguuccuguggugcucaccugacccugcaggguacccugcaggcaggaaaagaaaaaagaggcggcgggggaagaaguccuucagggauccccggggaaaccuggaggaagucgaggucggggggguacagcugcuucggucgccgcggaucacuggcguccagcggagaaacaggagcucgacuccuuccuuucgcugcuggcuccacauccgaucgucgcugaagcgccacgcccccucgcccggagcuucucucuggcuacuacccgguggauacaacugaagcuucaaacccccaaaccgcuuuucuuacaucuucacugcucuccgggcgagggugaaagcccucggaacccuagauugggucccu

SEQ ID NO: 78 - RNA, 385 nt, Pospiviroid
  JQ974378.1 Tomato chlorotic dwarf viroid clone 1, complete genome
gaauucgcccuucccugaagaagcgcuccuccgagccgccuucuuuuuucuuuucugcucaggaggucaggugugaaccacaggaaccacgaguuuaguuccgaggaaccaacugcgguuccaagggcuaaacacccucgccccgaagcaaguaagauagagaaaaagcgguucucgggagcuucaguuguuuccaccggguaguagccgaagcgacagcgcaagggggcgaggggucgaguggcgcgcaaaggaaggaagccgcgucagaaaggaagggugaaaacccuguuucucgcgggcuuacuccuguuucgccuuccacaagcucccugccgccuuuugccaguucgcuccagguuuccccggggauaagggcgaauuc

SEQ ID NO: 79 - RNA, 380 nt, Pospiviroid
  MH771135.1 Citrus exocortis viroid isolate CEVd-CY218, complete genome
gggaucuuucuugagguuccuguggugcucaccugacccugcaggcaguaaaagaaaaaagaggcggcgggggaaaaaguccuucagggggaggguaucuaagaccuugugacauaugcggggagaaguggaagugugccugccauucaaaagauuuuggaucacuggcguccagcggagaaacaggagcucgucuccuuccuuucgcugcuggcuccacauccgaucgucgcugaagcgccacgcccccucgcccggagcuucucucuggcuacuacccgguggauacaacugaagcuucaaccccaaaccgcuuuucuuauaucuucacugcucuccgggcgagggugaaagcccucggaacccuagauugggucccu

SEQ ID NO: 80 - RNA, 375 nt, Apscaviroid
  MG780426.1 Grapevine yellow speckle viroid 2 clone 2, complete genome
acuaguacuuucuucuaucuccgaagccgguuugaggcccggcgaaacgcgggccugucuccuaagaugccuccgcuagucgagcggacuuggccucuuccgcccgaggaccuuuucuuucugaucuugcuuguuccaacgagccccgcgaguggaauccccggaaccccugcgaaaaaggguccucggaucauuuuccuugugguuccugugguuacaccucggaaggccuccgcggaccugcagagaaaagaagaagggcccagaggggaaugagccucgucgucgacgaaggggugcauuccgaagagcuggccuggcgucguccggcucccucgaccgcaccggagcgcuagaaaagguccucggacuagu

SEQ ID NO: 81 - RNA, 375 nt, Apscaviroid
  KY404214.1 Australian grapevine viroid isolate AGVd-Iran6, complete genome
ugggcaccaacuagugguuccugugguucacaccgaaggccgcgaagcuaggaagaaaaagauagaaaagcuggguaagacucaccuggcgacucgucgucgaggucgacgaaggguccucagcagagcaccggcaggaggcgcuaugcaggaacgcuagggguccuccagcggaggacugaagaaacuccgguuucuucuuucacucuguagcuggaaucccuguugcgcuugcuggcgaaaccugcagggaagcuagcugggucccgcuagucgagcggacucgucccagcggucccaaccaguuuucuuuauccuauuuuucccugcgggcgcccggucgugguuacccuggaacccccuguuuggaggccc

SEQ ID NO: 82 - RNA, 375 nt, Apscaviroid
  HM211854.1 Australian grapevine viroid isolate F3, complete genome
gucgacgacgagucgccaggugagucuuacccagcuuuucuaucuuuuucuuccuagcuucgcggccuucggugugaaccacaggaaccacuaguuggugcccagggccuccaaacaaggggauccagggaaaccacgaccgggcgcccgcagggaaaaauaggauaaagaaaacugguugggaccgcugggacgaguccgcucgacuagcgggacccagcuagcuucccugcagguuucgccagcaagcgcaacagggauuccagcuacagagugaaagaagaaaccggaguuucuucaguccuccgcuggaggaccccuagcguuccugcauagcgccuccugccggugcucugcugaggacccuucgucgac

SEQ ID NO: 83 - RNA, 375 nt, Apscaviroid
  AB429168.1 Apple fruit crinkle viroid RNA, Apscaviroid central conserved region, strain: hop isolate, clone: HY-A25
ugggcuccaacuagugguuccugugguuucacaccgaaggccgcgaagcuuggaaagaaaaagauagaaaaucuggguaagacucaccuggugacucgucgucgacgaaggguccucagcagagcaccggcaggcgcggcgaaguggagaacgcuagccguccuccaguggaagaacuggaaaagcuccggcuuuuccuuuuuucuguuacuggaacgcgcuguugcgcucacuagccagaccugcagggaaucuagcugggaaccugcuagucgagcggacucgucccagcggucccaaccaguuuuucuuuauccuauuuuucccugcgagcgcccggucguggauaccuaggauacccuuguuugggugccc

SEQ ID NO: 84 - RNA, 375 nt, Pospiviroid
  AY372393.1 Citrus exocortis viroid isolate 89002600, complete genome
ggaaaccuggaggaagucgaggucgggggggaacagcugcuucggucgccgcggaucacuggcguccagcggagaaacaggagcucgucuccuuccuuucgcugcuggcuccacauccgaucgucgcugaagcgccacgcccccucgcccggagcuucucucuggcuacuacccgguggauacaacugaagcuucaaccccaaaccgcuuuucuuauaucuucacugcucuccgggcgagggugaaagcccucggaacccuagauugggucccucgggaucuuucuugagguuccugcuggggggcucacccgaaccugugcgcgcggaaaagaaaaaaaggggggggggggaaaaaccguucagggauccccgg

SEQ ID NO: 85 - RNA, 374 nt, Pospiviroid
  KY810771.1 Columnea latent viroid strain FERA_160205, complete genome
cggaacuaaacucgugguuccugugguucacaccugacccugcagccaugcaaagaaaaaaagauugggcggaagagcgcaagagcggucucaggagccccggggcaacucagaccgagcggggucggaggaucuucuggagcggggcccugcuucagacaggaguaauccccgcugaaacaggguuuucacccuuccuuucuucggguuuccuuccucuguugcggaccucgcccggagucuucucgaccagcgcagguucugacgcgaccgguggcaucaccgaguugcucaagccucaaccgccuuuuucucuauucugcgcuuggucuccgggcgaggguguuuagcccuuggaaccgcaguugguuccu

SEQ ID NO: 86 - RNA, 374 nt, Pospiviroid
  HG739076.1 Citrus exocortis viroid, complete sequence, isolate 11/0171/VI.7
cgggaacuuucuugagguuccuguggugcucaccugacccugcaggcaguaaagaaaaaagaacggcggcgagggaagaaguccuucagggauccccggggaaaccuggaggaaguugaggucgggggagcagcggcugcuucggucgccgcggaucacuggcguccagcggagaaacaggagcucgucuccuuccuuucgcugcuggcuccacauccgaucgucgcugaagcgccucgcccccucgcccggagcuucucucuggagacuacccgguggauacaacugaagcuucaaacccaagccgcuuuuucuuaaucuucucugcucuccgggcgagggugaaagcccuuggaacccuagauugggucccu

SEQ ID NO: 87 - RNA, 374 nt, Pospiviroid
  M30870.1 Citrus exocortis viroid (CEV-JB) complete genome
cgggaucuuucuugagguuccuguggugcucaccugacccugcaggcagaaaaagaaaaagauggcggguggggaagaaguccuucagggauccccggggaaaccuggaggaagucgaggucggggggagcuucugccucggucgccgcggaucacuggcguccagcggagaaacaggagcucgucuccuuccuuucgcugcuggcuccacaccuccgaucgucgcugaggcgucgccaccccucgcccggagcuucucucuggauacuacccgguggauacaacugaagcuucaaccccaguaccgcuuuucuugaucucuacugcucuccgggcgagggugaaagcccucggaacccuagauugggucccuc

SEQ ID NO: 88 - RNA, 373 nt, Apscaviroid
  LC199972.1 Apple fruit crinkle viroid genomic RNA, complete genome, clone: HS1-T
ugggcuccaacuagugguuccuaugguuacacaccgaaggccgcgaagcuaggaaagaaaaagauaagaaagcuggguaagacucaccuggugacucgucgucgacgaaggguccucagcagagcaccggcaggcgcggcgaugcggagaacgcuagcguguccuccaguggagaacuggaaaagcuccggcuuuuccuuuuuucuguaacuggaacgcgcuguugcgcucgcuagccagaccugcagggaagcuagcugggucccgcuagucgagcggacucgucccagcggucccaaccaguuucuuuuauccuauuuuucccugcgggcgcccgguuguggaugccuaggauucccuuguuugggugccc

SEQ ID NO: 89 - RNA, 373 nt, Pospiviroid
  HM043818.1 Columnea latent viroid isolate 14 clone 2, complete genome
cggaacuaaacucgugguuccugugguacacaccugacccugcagccaugcaaaagaaaaaagaacgggagggagagcgcaagagcggucucaggagccccggggcaacucagaccgagcggggucucguggucgagggcguagccccuguucagacaggaguaaucccagcugaaacaggguuuucacccuuccuuucuucugguuuccuuccucugcuucagcggccucgcccggaaccucuugaccagcgcaggugcugacgcgaccgguggcaucaccgaguuugcucaagccucaaccuccuuuuucucuaucugaagcuuggucuccgggcgaggguguuuagcccuuggaaccgcaguagguuccu

SEQ ID NO: 90 - RNA, 373 nt, Pospiviroid
  AY367350.1 Columnea latent viroid isolate 89001013, complete genome
cggaacuaaacucgugguuccugugguucacaccugacccugcagccaugcgaagaaaaaagaacgggagaagaaagagcaagagcggucucaggagccccggggcaacucagaccgagcggggagagcggacgacugggcgggggccuccucagacaggguguaaucccagcugaaacaggguuuucacccuuccuuucuucggguuuccuuuccucagcuggcggccucgcccggagucuucuucuccagcgcagguucugacgcgaccgguggcaucaccgaguuucgcucgaaccucaaucuccuuuuucucuaucuugcuuggucuccgggcgaggguguuuagcccuuggaaccgcaguugguuccu

SEQ ID NO: 91 - RNA, 373 nt, Pospiviroid
  X95292.1 Columnea latent viroid-B stem-loop RNA
cgggaucauucucgugguuccugugguucacaccugacccugcaggccaucgaaaagaaaaaagaacgggagggagagcgcaagagcggucucaggagccccggggcaacucagaccgagcgaggucucguggucgagggcguucggaucauuccagacagguguaaucccagcugaaacaggguuuucacccuuccuuucuucugguuuccuuccucuggaaggucuuccgcccggaaccucaagaccagcgcgggugcugacgcgaccgguggcaucaccgagauugcucaagccucaaccuccuuuuucucuauucugacguuggucuccgggcgaggguguuuagcccuuggaaccgcaguuggucccu

SEQ ID NO: 92 - RNA, 372 nt, Pospiviroid
  KY110721.1 Citrus exocortis viroid strain R140902-18, complete genome
gaucacuggcguccagcggagaaacaggagcucgacuccuuccuuucgcugcuggcuccacauccgaucgucgcugaagcgccacgcccccucgcccggagcuucucucuggcuacuacccgguggauacaacugaagcuucaaccccaaaccgcuuuucuuguaucuucacugcucuccgggcgagggugaaagcccucggaacccuagauugggucccucgggaucuuucuugagguuccuguggugcucaccugacccugcaggcaggaaaagaaaaaagaggcggcgggggaagaaguccuucagggauccccggggaaaccuggaggaagucgaggucgggggggaucaacagcuucggucgccgcg

SEQ ID NO: 93 - RNA, 372 nt, Pospiviroid
  KY473932.1 Citrus exocortis viroid isolate Ex-SR-Ver, complete genome
uccggggauccccugaaggacuucuuccccacacgaaaucuuuuucuuuuucugccugcaggguaaggugagcaccacaggaaccucaagaaaguucccgagggacccagucuaggguuccgagggcuuucacccucgcccggagagcaguagagaucaagaaaagcgguucugggguugaagcuucaguuguauccaccggguaguagccagagagaagcuccgggcgagggguggcgacgccucagcgacgaucggauguggagccagcagcgaaaggaaggagacgagcuccuguuucuccgcuggacgccaggauccgcggcgaccgaggcagagcuccacccgaccucgacuucccccagguuucca

SEQ ID NO: 94 - RNA, 372 nt, Pospiviroid
  S67441.1 viroid genome [citrus exocortis viroid CEV, isolate tomato hybrid, CEVt, Genomic Complete, 372 nt]
cgggaacuuucuugaggucccuguggugcucaccugaccccgcaggcagaaagagaaagaagaggcggcuggggaagaaguccuucagggauccccggggaaaccuggaggaagucgaggucgggggggaucagcugcuucggucgccgcggaucacuggcguccagcggagaaacaggagcucgucuccuuccuuucgcugcuggcuccacauccgauugucgcugaagcgccucgcccccucgcccggagcuucucucuggagacuacccgguggaaacaacugaagcuucgaccccaaaccguuuuccuuauaucuucacugcucuccgggcgagggugaaagcccuuggaacccuagauugggucccu

SEQ ID NO: 95 - RNA, 372 nt, Pospiviroid
  DQ431992.1 Citrus exocortis viroid isolate CEVd-XNM-HJ Guangdong-YC, complete genome
ggaaaccuggaggaagucgaggucgggggggaaccgcugcuucacccacagcgcaucacuggcguccagcggagaaacaggagcucgucuccuuccuuucgcugcuggcuccacauccgaucgucgcugaagcgccacgcccccucgcccggagcuucucucuggcuacuacccgguggauacaacugaagcuucaaccccaaaccgcuuuucuuauaucuucacugcucuccgggcgagggugaaagcccucggaacccuagauugggucccucgggaucuuucuugagguuccuguggugcucaccugacccugcaggcaggaaaagaaaaaagaggcggcgggggaagaaguccuucaggguuccccgg

SEQ ID NO: 96 - RNA, 372 nt, Pospiviroid
  X53716.1 L.esculentum citrus exocortis viorid-tomato (CEVd-t) RNA
cgggaucuuuccugagguuccuguggugcucaccugacccugcagccagaauaagaaaaaagaauggcggaaggggaagaaguccuucagggauccccggggaaaccuggaggaagucgaggucggggggucggacugcugcggucgccgcggaucacuggcguccagcggagaaacaggagcucgucuccuuccuuucgcugcuggcuccaucauccgaucgucgcugcaggcucuucgccccucgcccggagcuucucucuggagacuacccgguggaaacaacugaagcuucaacccaaagccgcuuuuucucuaucuuagcuggucuccgggcgagggugaaagcccguggaacccugguaggucccu

SEQ ID NO: 97 - RNA, 371 nt, Apscaviroid
  AB429216.1 Apple fruit crinkle viroid RNA, Apscaviroid central conserved region, strain: apple isolate, clone: AA-J14
ugggcuccaacuagugguuccugugguuucacaccgaaggccgcgaagcuuggaaagaaaagaaacaaagcuggguaagacucaccuggugacucgucgucgacgaaggguccucagcagagcuccggcaggcgcggcgaagcggagaacgcuagccguccuccaguggagaacuggaaaagcuccggcuuuuccuuuuuuucuauaacuggaacgcgcuguugcgcucgcuagccagaccugcagggaagcuggcugggucccgcuagucgagcggacucgucccggcggucccaaccaguuuuauuuuucuaauuuuucccugcgagcgcccggucguggauaccuaggauucccuuguuugggugccc

SEQ ID NO: 98 - RNA, 371 nt, Pospiviroid
  KR020040.1 Iresine viroid 1 isolate Cp4, complete genome
ugugguuccaaugguugcaccccugaccugcaaugcaaaaagaaaaaaagauggggcggcggcaacaacgcuucagggauccccggggaaaccuggagcgaacucggcaaggaggcuuggcgguaggugcgcggagucgaccgcguaaagaccggagaacuccucgcggcggaagaaacaggagcucgucuccuuccuuucugucgcccgcaggacucacucggcagcggcgucuucuccgcacccucgcccgcuuccgcgcuggucgcucuggcuacuacccgguggauacaacuguagcuugaagccugcccgcccuuuuucuucuaucuucugcugcgcggcgagggugggcuucuaaaggaaacccu

SEQ ID NO: 99 - RNA, 371 nt, Pospiviroid
  JF446930.1 Columnea latent viroid isolate CM10-6c2, complete genome
cggaacuaaacucgugguuccugugguucacaccugacccugcagccaugcaaagaaaaaaagaacgggaggaagagcgcaagagcggucucaggagccccggggcaacucagaccgagcggggaucgcggaccgagggcggaagccugcuucagacaggaguaaucccugcagaaacaggguuuucacccuuccuuucuucggguuuccuuccucugcuugcggccucgcccggagucuucgaauccagcgcagguucugacgcgaccgguggcaucaccgaguugcucaagccucaaccuccuuuuucucauuucuacgcuuggucuccgggcgaggguguuuagcccguggaaccgcaguugguuccu

SEQ ID NO: 100 - RNA, 371 nt, Pospiviroid
  EF126047.1 Citrus exocortis viroid isolate LMPE7, complete genome
cgggaucuuucuugagguuccuguggugcucaccugacccugcaggcaguaaaagaaaaaagaggcgggggggaaagaaguccuucagggauccccggggaaaccuggaggaagucgaggucggggggagcaacugccucggucgccgcggaucacuggcguccagcggagaaacaggagcucgacuccuuccuuucgcugcuggcuccacauccgaucgucgcugaggccugcgcgccccucgcccggagcuucucucuggagacuacccgguggaaacaacugaagcuucaaccccguaccgcuuuucuugauucucagcugcucuccgggcgagggugaaagcccucggaacccuagagugggucccu

SEQ ID NO: 101 - RNA, 371 nt, Pospiviroid
  EF015581.1 Columnea latent viroid, complete genome
cggaacuuuucucgugguuccugugguuacacaccugacccugcagccaugcaaagaaaaaagaacggcggagagagcgcaagagcggucucaggagccccggggcaacucagaccgagcggggaucgcggaccgagggcggaagccugcuucagacaggaguaauccccgcugaaacaggguuuucacccuuccuuucuucggguuuccuuccucugcuugcggccucgcccggagucuucgaauccagcgcagguucugacgcgaccgguggcaucaccgaguuugcucaagccucaauccgcuuuuucuucaacuuagcuuggucuccgggcgagggugaaaagcccguggaaccgcagaagguuccu

SEQ ID NO: 102 - RNA, 371 nt, Pospiviroid
  DQ431994.1 Citrus exocortis viroid isolate CEVd-Italy-BL China, complete genome
ggaaaccuggaggaagucgaggucgggggggacgacugccucggucgccgcggaucacuggcguccagcggagaaacaggagcucgucuccuuccuuucgcugcuggcuccacauccgaucgucgcugaggcuucgcgcccccucgcccggagcuucucucuggcuacuacccgguggauacaacugaagcuucaaccccaaaccgcuuuucuuaaaucuucacugcucuccgggcgagggugaaagcccucggaacccuagagugggucccucgggaucuuucuugagguuccugugguucucaccugacccugcaggcaggaaaagaaaaaagaggcgccuggggaagaaguccuucagggauccccgg

SEQ ID NO: 103 - RNA, 370 nt, Pospiviroid
  JF742633.1 Columnea latent viroid clone Solanum 4, complete genome
cggaacuaaacucgugguuccugugguucacaccugacccugcagccaugcaaagaaaaaaagaacgggaggaagagcgcaaagagcggucucaggagccccggggcaacucagaccgagcggggucuugaccaguggcgagcgcccuguucagacaggaguaaucccagcagaaacaggguuuucacccuuccuuucuucugguuuccuuccucugcuucagcggccucgcccggagucuugaccagcgcagguucugacgcgaccgguggcaucaccgaguucgcucaagccucauaccuccuuuuucuucauucuagcuuggucuccgggcgaggguguuuagcccuuggaaccgcaguugguuccu

SEQ ID NO: 104 - RNA, 370 nt, Pospiviroid
  KY646193.1 Citrus exocortis viroid isolate PTZ57R/T, complete genome
ucggucgccgcggaucacuggcguccagcggagaaacaggagcucgacuccuuccuuucgcugcuggcuccacauccgaucgucgcugaggccugcgcgccccucgcccggagcuucucucuggcuacuacccgguggauacaacugaagccucaaccccguaccgcuuuucuugauucucagcugcucuccgggcgagggugaaagcccucggaacccuagagugggucccucgggaucuuucuugagguuccuguggugcucaccugacccugcaggcaguaaaagaaaaaagaggcgggggggaagaaguccuucagggauccccggggaaaccuggaggaagucgaggucggggggagcaacugcc

SEQ ID NO: 105 - RNA, 369 nt, Pospiviroid
  KY473931.1 Citrus exocortis viroid isolate Ex-CA-Ver, complete genome
cccugaaggacuucuuccccacccgccauacguuaaacgugaggcacacagaggcacaggagcaccacaggaacaccaagaaagaucccgagggacccacucuaggguuccaaaggcuuucacccucgcccggagagcagcacagaucaacaaaagcggcucugggguagaagcuucaguuguauccaccggguaguagccagagagaagcaccgggcgagggguggcgacgccucagcgacgaucggauguggagccagcagcgaaaggaaggaguagagcucaucuuuuccgcugaccccaggauccgcggcgaacgaggcagaacccaacccagacaccgacuuccuccagguuuccccggggaua

SEQ ID NO: 106 - RNA, 369 nt, Pospiviroid
  DQ471994.1 Citrus exocortis viroid isolate CSC09, complete genome
cgggaucguucuugagguuccuguggugcucaccugacccugcaggcagaaaaagaaaaagaggugggcggggaagaaguccuucugggauccccggggaaaccuggaggaagucgaggucggggggagcuucugccucggucgccgcggaucacuggcguccagcggagaaacaggagcucgacuccuuccuaucucugcugccuccacauccggucgucgcugaggcgucgccaccccucgcccggagcuucucugugcuuaauagccgguggauacaacugaagcuucaaccccaaaagcagguucuugaucuccgaggcucuccggccgagggugaaagcccucggaacccucgaaagggucccu

SEQ ID NO: 107 - RNA, 369 nt, Pospiviroid
  Y00328.1 Citrus exocortis viroid (CEV-g) RNA genome
cgggaucuuucuugagguuccuguggugcucaccugacccugcaggagaaagagaaaaagaggcggcgggggaagaaguccuucagggauccccggggaaaccuggaggaagucgaggucgggggggacuacugcuugggcggccgcggaucacuggucugcagcggagaaacaggaccugguguccuuccuuucgcugcuggcuccacauccgaucgucgcugaagcguugcgcccccucgccgggagcuucucucuggagacuacccgguggacacuacugaagcuuuaaccccaagccgcuuuucuuauaucuucacuguucuccgggccagggugaaagccccccgaacccuagauugggacccu

SEQ ID NO: 108 - RNA, 368 nt, Apscaviroid
  MG010378.1 Grapevine yellow speckle viroid 1 isolate Volovnik AV5, complete genome
ucuccggaucuucuugcuugugguuccugugguuucaccucggaaggccgccgcgguccugcaaagaagaagauaggggcagagggggagugagccucgucgucgacgaaggggugcacuccgagugccugagcuggucgacguccagcucccucgggaccacgcugcucugggcggaagagucuucugacacuucuagccuauucagcuucgcguccuugaggccuggcguaacgcggcucuugccaccaggaugccuccgcuagucgagcggacuuggucucuuuccgcccaaagcccuuuuucuuucaacugagcuuguaccaacgcgccccgcgagugcaauccccggaacccccgcuaagagg

SEQ ID NO: 109 - RNA, 368 nt, Apscaviroid
  MF510389.1 Grapevine yellow speckle viroid 1 isolate GYSV1-HUHT, complete genome
caccucggaaggccgccgcggaccugcaaagaagaagauaggggcagagggggaaugagccucgucgucgacgaaggggugcauuccgagugccugagcuggucgacguccagcucccucgggaccacgcugcucugggcggaagagucuucugacuuuucuagccuauucagcuucgcguccuugaggccuggcguaacgcggcucuugccaccaggaagccuccgcuagucgagcggacuuggucucuauccgcccaaagcccuuuuucuuucaacugagcuuguaccaacgcgccccgcgagugcaauccccggaacccccgcuaagaggucuccggaucuucuugccugugguuccugugguuu

SEQ ID NO: 110 - RNA, 367 nt, Apscaviroid
  LC500206.1 Japanese grapevine viroid Na RNA, complete genome
cgggacaaacacuugugguuccugugguucaccucuucgauccccuaccugcgaaagaaaaagagugagaagagcgccagaggcuucggagcgacucgucgucgacgaagggccucuccgagcaggccucccugccgaguccggggcuggagcucuggaucgccgauggacagagagaggcucugccuucuucuccagucgcggaaccaguugucucucccccgcugcucugguaaggcgccgacucgagagcccgcuagucgagcggacucguccuccuccgcccgaggcgcuuuuucuuuucuccuauucuguagcagcggggcgagaguguguuuacccuggaacccacugguuguggguccuu

SEQ ID NO: 111 - RNA, 367 nt, Apscaviroid
  MH476216.1 Grapevine yellow speckle viroid 1, complete genome
caauccccggaacccccgcuaagaggucuccggaucuucuugcuugugguuccugugguuucaccucggaaggccgccgcggaccugcagaaaagaagauaggggcagaggggguucgagccucgucgucgacgaaggggugcgaaccgagugccugagcuggucgacguccagcucccucgggaccacgcugcucugggcggaagagucuucugacacuucuagccuauucagcuucgcguccuugaggccuggcguaacgcggcucuugccaccaggucgccuccgcuagucgagcggacuuggucucuuccgcccaaagcccuuuuucuuacugaucagcuuguuccaacgcgccccgcgagug

SEQ ID NO: 112 - RNA, 367 nt, Apscaviroid
  KX966270.1 Grapevine yellow speckle viroid 1 clone 9s CZZ, complete genome
cggaucuucuugcuugugguuccugugguuucaccucggaaggccgccgcggaccugcagagaagaagauaggggcagaggggguucgagccucgucgucgacgaaggggugcgaaccgagugccugagcuggucgacguccagcucccucgggaccacgcugcucugggcggaagagucuucugacacuucuagccuauucagcuucgcguccuugaggccuggcguagcgcggcucuugccaccaggucgccuccgcuagucgagcggacuuggucucuuccgcccaaaacccuuuuucuuucuaaucagcuuguuccaacgcgccccgcgagugcaauccccggaacccccgcuaagaggucuc

SEQ ID NO: 113 - RNA, 367 nt, Apscaviroid
  KT000352.1 Grapevine yellow speckle viroid 1 isolate 22_2_3, complete genome
  misc_feature (15)..(21): n is a, c, g, or u
  misc_feature (91)..(104): n is a, c, g, or u
  misc_feature (123)..(127): n is a, c, g, or u
  misc_feature (139)..(146): n is a, c, g, or u
  misc_feature (171)..(179): n is a, c, g, or u
  misc_feature (190)..(192): n is a, c, g, or u
  misc_feature (252)..(254): n is a, c, g, or u
  misc_feature (269)..(289): n is a, c, g, or u
  misc_feature (325)..(334): n is a, c, g, or u
  misc_feature (343)..(344): n is a, c, g, or u
  misc_feature (359)..(360): n is a, c, g, or u
cuggggaauucucgnnnnnnnaguugccgcauacuuaccugagaaaggagccccggggcaacucuucucagaauccagcgagaggcguagnnnnnnnnnnnnnngagagagggccgcggugcnnnnnucuggaguagannnnnnnnggcuucuucgaaacaccaucgaucnnnnnnnnngucccuucuunnncuuuuaccaggaugccuccgcuagucgagcggacuuggucucuuccaccgguggcaucannnccucucgguucgucnnnnnnnnnnnnnnnnnnnnnuuccaaccugcuuuuuuucuaucugagccucugccnnnnnnnnnngcggauccnnucucuugagccccunnuaagagg

SEQ ID NO: 114 - RNA, 367 nt, Apscaviroid
  LT601587.1 Grapevine yellow speckle viroid 2, complete sequence, isolate KO
cgagugccugagcuggucgacguccagcucccucgggaccacgcugcucugggcggaagagucuucugacacuucuagccuauucagcuucgcguccuugaggccuggcguaacgcggcucuugccaccagguugccuccgcuagucgagcggacuuggucucuuccgcccaaagcccuuuuucuuucuaaucagcuuguuccaacgcgccccgcgagugcaauccccggaacccccgcuaagaggucuccggaucuucuugcuugugguuccugugguuucaccucggaaggccgccgcggaccugcaaagaagaagauaggggcagaggggguuugagccucgucgucgacgaaggggugcaaac

SEQ ID NO: 115 - RNA, 367 nt, Apscaviroid
  KU880715.1 Grapevine yellow speckle viroid 1 isolate SY-BR, complete sequence
cuuguuccaacgcgccccgcgagugcaauccccggaacccccgcuaagaggucuccggaucuucuugcuugugguuccugugguuucaccucggaaggccgccgcggaccugcagagaagaagauaggggcagaggggguuugagccucgucgucgacgaaggggugcaaaccgagugccugagcuggucgacguccagcucccucgggaccacgcugcucugggcggaagagucuucugacacuucuagccuauucagcuucgcguccuugaggccuggcguaacgcggcucuugccaccuggcugccuccgcuagucgagcggacuuggucucuuccgcccaaagcccuuuuucuuucuaaucag

SEQ ID NO: 116 - RNA, 367 nt, Apscaviroid
  KF007320.1 Grapevine yellow speckle viroid 2 isolate 6703_YS2_ThS2, complete genome
cggaucauuuuccuugugguuccugugguuacaccucggaaggccuccgcggaccugcagagaaaagaagaagggcccagaggggaaugagccucgucgucgacgaaggggugcauuccgaagagcuggccuggcgucguccggcucccucgaccgcaccggagcgcuagaaaagguccucggacuuucuucuaucuccggaagccgguauuugaggcccggcgaaacgcgggccugucuccuaagaugccuccgcuagucgagcggacuuggccucuuccgcccgaggaccuuuucuuucugaucuugcuuguuccaacgagccccgcgaguggaauccccggaaccccugcgaaaaaggguccuc

SEQ ID NO: 117 - RNA, 367 nt, Apscaviroid
  DQ371477.1 Grapevine yellow speckle viroid 1 clone Tu CGVd 1-17, complete genome
ucuccggaucuucuugcuugugguuccugugguuacaccucggaaggccgccgcggaccugcagagaagaagauaggggcagaggggguucgagccucgucgucgacgaaggggugcgaaccgagugccugagcuggucgacguccgacucccucgagaccugcugcucugggcggaagagucuucgacuccuucuuugccuauucagcuucgcgucucugaggcucggcguaacgcggcucuugccaccagguugccuccgcuagucgagcggacuuggucucuuccgcccaaagcccuuuuucuuucuaaucagcuuguuccaacgcgccccgcgaguacaauccccggaacccccgcuaagagg

SEQ ID NO: 118 - RNA, 367 nt, Apscaviroid
  AB028465.1 Grapevine yellow speckle viroid 1 mRNA, the sequence of yellow speckle symptom
uccucggaucacuuuccugugguuccugugguuacaccucggaaggccgccgcggaccugcaaagaagaagauaggggcagagggggagugagccucgucgucgacgaaggggugcacuccaaagucccgaacuggcgucguccggcucuccucggagccucgcugcucugggcggaagagucuucugacuuuuucuagccuauucagcauugcgcucuugaggcccggcgaaacgcgguucgucugcugaggaugccuccgcuagucgagcggacuuggucucuuccgcccaaagcccuuuuucuuucaacugagcuuguuccaacgcgccccgcgaguggaauccccggaaccccugcaaagagg

SEQ ID NO: 119 - RNA, 366 nt, Apscaviroid
  MK804769.1 Grapevine yellow speckle viroid 1 isolate GYSV1.NUB-BR, complete genome
uaagaggucuccggaucuucuugcuugugguuccugugguuucaccucggaaggccgccgcggaccugcaaagaagaagauaggggcagagggggagugagccucgucgucgacgaaggggugcacuccgagugccugagcuggucgacguccagcucccucgggaccugcugcucugggcggaagagucuucugacacuucuagccuauucagcuucgcguccuugaggccuggcguaacgcggcucuugccaccaggaugccuccgcuagucgagcggacuuggucucuuccguagucgagcggacuuggucucuuccgcccaaagcccuuucgccccgcgagugcaauccccggaacccccgc

SEQ ID NO: 120 - RNA, 366 nt, Apscaviroid
  KU880716.1 Grapevine yellow speckle viroid 1 isolate TR-BR, complete sequence
ugcugcucugggcggaagagucuucugacacuucuagccuauucagcuucgcguccuugaggccuggcguaacgcggcucuugccaccaggaugccuccgcuagucgagcggacuuggucucuuccgcccaaagcccuuuuucuuucaacugagcuuguaccaacgcgccccgcgagugcaauccccggaacccccgcuaagaggucuccggaucuucuugcuugugguuccugugguuucaccucggaaggccgccgcggaccugcaaagaagaagauaggggcagagggggagugagccucgucgucgacgaaggggugcacuccgagugccugagcuggucgacguccagcucccucgggacc

SEQ ID NO: 121 - RNA, 366 nt, Apscaviroid
  KU880714.1 Grapevine yellow speckle viroid 1 isolate MH-BR, complete sequence
uucuuucaacugagcuuguuccaacgcgccccgcgagugcaauccccggaacccccgcuaagaggucuccggaucuucuugcuugugguuccugugguuucaccucggaaggccgccgcggaccugcaaagaagaagauaggggcagagggggagugagccucgucgucgacgaaggggugcacuccgagugccugagcuggucgacguccagcucccucgggaccugcugcucugggcggaagagucuucugacacuucuagccuauucagcuucgcguccuugaggccuggcguaacgcggcucuugccaccaggaugccuccgcuagucgagcggacuuggucucuuccgcccaaagcccuuu

SEQ ID NO: 122 - RNA, 366 nt, Apscaviroid
  KU880713.1 Grapevine yellow speckle viroid 1 isolate RM-BR, complete sequence
ccggaaccccugcaaagagguccucggaucacuuuccugugguuccugugguuacaccucggaaggccgccgcggaccugcaaagaagaagauaggggcagagggggagugagccucgucgucgacgaaggggugcacuccaaagcuccgaacuggcgucguccggcucuccucggagccucgcugcucugggcggaagagucuucugacuuuucuagccuauucagcauugcgcucuugaggcccggcgaaacgcgguucgucugcugaggaugccuccgcuagucgagcggacuuggucucuuccgcccaaagcccuuuuucuuucaacugagcuuguuccaacgcgccccgcgaguggaaucu

SEQ ID NO: 123 - RNA, 366 nt, Pospiviroid
  AY372390.1 Citrus exocortis viroid isolate 8900808, complete genome
cgggaucuuucuugagguuccuguggugcucaccugacccugcaggcaggaaaagaaaaaagaggcggcggcggggaaaccuggaggaagucggaaaccuggaggaagucgaggucggggggguacagcugcuucggucgccgcggaucacuggcguccagcggagaaacaggagcucgucuccuuccuuucgcugcuggcuccacauccgaucgucgcugaagcgccacgcccccucgcccggagcuucucucuggcuacuacccgguggaaacaacugaagcuucaaccccaaaccgcuuuucuuauaucuucacugcucuccgggcgagggugaaagcccucggaacccuagauugggucccu

SEQ ID NO: 124 - RNA, 365 nt, Pospiviroid
  HQ667139.1 Tomato apical stunt viroid isolate sj3, complete genome
cgggaacuuucuugagguuccuguggugcucaccugacccugcaggcaucaagaaaaaagaauggcgcggaggagaagaaguccuucagggauccccggggaaaccuggaggaagucgaggucgggggcuucggacuacuccuucgugagacaggaguaauucccgccgaaacaggguuuaucacccuuccuuucuucggguguccuuccucucgccuggagaggucuucggcccucgcccggagcuucucucuggagacuacccgguggaaacaacugaagcuucaacccucucgcgcuuuuucucuaucuuuguugcucuccgggcgagggugaaagcccguggaacccuggaaggagucccu

SEQ ID NO: 125 - RNA, 364 nt, Coleviroid
  NC_003683.1 Coleus blumei viroid 3, complete genome
cggaucauuuuccuugugguuccugugguacaccuccgaaggcgcgcucggcgagaagagaguaaauagaagagagagcuuaccuggguucccuggcagcgcugcaacggaauccagggcguuggccccgauggaguuuccgaugagcaccggugagaagaaacugucucgcagaauagaagucucuucuuguuugcuuugagacuucuucuccacagggcucuucggcucuucgcggguacccagcaaccugcucccacgcggcgauugcaaaggacccgguucgcucuccuuuucuuauuuaccugcuucagaccgaccgccccggggagugaaucacuggaaccgcgagaaaaaggauccu

SEQ ID NO: 126 - RNA, 364 nt, Pospiviroid
  MG132058.1 Tomato apical stunt viroid isolate Touboudan, complete sequence
cgggaacuuuguugagguuccuguggugcucaccugacccugcaggcaucaagaaaaaagauauggcgccgggaggaagaaguccuucagggauccccggggaaacccuggaggaagucgaggucgggggcuucggaucauuccugguugagacaggaguaaucccagcugaaacaggguuuucacccuuccuuucuucugguuuccuuccucucgccggaaggucuucggcgcucgcccggagcuucucucuggagacuacccgguggaaacaacugaagcuucgacuccacggcucuuuuucucuaucuuuguugcucugggggcgagggugagagcccguggaucccuggaaggagucccu

SEQ ID NO: 127 - RNA, 364 nt, Pospiviroid
  MG132057.1 Tomato apical stunt viroid isolate Akumadan, complete sequence
cgggaacuuucuugagguuccuguggugcucaccugacccugcaggcaucaagaauaacuuuuccaggagcgggaggaagaaguccuucagggauccccgggggaaaccuggaggaagucgaggucgggggcuucggaucauuccugguugagacaggaguaaucccagcugaaacaggguuuucacccuuccuuucuucugguuuccuuccucucgccggaaggucuucggcccucgcccggagcuucucucuggagacuacccgguggaaacaacugaagcuuccacuccacgcucuuuuucucuaucuuuguugcucuccgggcgagggugaaagcccguggaacccuggaaggagucccu

SEQ ID NO: 128 - RNA, 364 nt, Pospiviroid
  KX579067.1 Tomato apical stunt viroid isolate 5458774, complete genome
cgggaacuuucuugagguuccuguggugcucaccugacccugcaggcaucaagaaaaaagauaggcgcgggagcgaagaaguccuucagggauccccggggaaacccgaaggaagucgaggacgggggcuuccgacuacucccgugugagacaggaguaaucccagcugaaacaggguuuucacccuuccuuucuucugguuuccuuccucucgccuggagaggucuucggcccucgcccggagcuucucuaaggagacuacccgguggauacaacugaagcuucaaccuccacgcgcuuuuucucuaucuuuguugcucuccgggcgagggugaaagcccguggaacccuaguaagagucccu

SEQ ID NO: 129 - RNA, 364 nt, Pospiviroid
  KJ956800.1 Citrus exocortis viroid isolate 2 from lemon, complete genome
cggggaagaaguccuucagggauccccggaaaccuggaggaagucgaggucgggggggaagaaguccuucagggauccccggggaaaccugaggaagucgaggucgggggggaacagcugcuucggucgccgcggaucacuggcguccagcggagaaacaggagcucgucuccuuccuuucgcugcuggcuccacauccgaucgucgcugaagcuucaaccccauaccgcuuuucuuaaaucuucacugcucuccgggcgagggugaaagcccucggaacccuagauugggucccucgggaucuuucuugagguuccuguggugcucaccugacccugcaggcaggaaaagaaaaaagaggcgg

SEQ ID NO: 130 - RNA, 364 nt, Pospiviroid
  KF437621.1 Tomato apical stunt viroid clone TASVd_31, complete genome
ucacccuuccuuucuucggguguccuuccucucgccuggagaggucuucggcccucgcccggagcuucucucuggagacuacccgguggaaacaacugaagcuucaucccucucgcgcuuuuucucuaucuuuguugcucuccgggcgagggugaaagcccguggaacccuggaaggagucccucgggaacuuucuugagguuccuguggugcucaccugacccugcaggcaucaagaaaaaagaauggcgcggaggagaagaaguccuucagggauccccggggaaaccuggaggaagucgaggucgggggcuucggacuacuccuucgugagacaggaguaauucccgccgaaacaggguuu

SEQ ID NO: 131 - RNA, 364 nt, Pospiviroid
  AY372394.1 Potato spindle tuber viroid isolate 94005977, complete genome
cggaacuaaacucgugguuccugugguucacaccugaccuccugagcagaaaaagaaaaaagaaggcggguucgcuccagguuuccccggggauauccccggggaaaccuggagcgaacuggcaauaaggacgguggggagugcccagcggccgacaggaguaauucccgccgaaacaggguuuucacccuuccuuucuucggguguccuuccucgcgcccgcaggaccaccccucgcccccuuugcgcugucgcuucggcuacuacccgguggaaacaacugaagcucccgagaaccgcuuuuucucuaucuucuuugcuucggggcgaggguguuuagcccuuggaaccgcaguugguuccu

SEQ ID NO: 132 - RNA, 363 nt, Pospiviroid
  MK286920.1 Tomato apical stunt viroid isolate Al-Kharj-SA2, complete genome
agggacuccuuccaggguuccacgggcuuucacccucgcccggagagcaacaaagauagagaaaaagcgcgagagagauugaagcuucaguuguuuccaccggguagucuccauauagaagcuccgggcaaaggccgaagaccuuccggcgagaggaaggaaaccagaagaaaggaaggaugaaaauccuguuucugcugggauuacuccugucucaaccaggaaugauccgaagcccccgaccucgacuuccuccagguuuccccggggaucccagaaggacuucuucuccuccgcgccuaucuuuuuucuugaugccugcagggucaggugagcaccacaggaaccucaagaaaguucccg

SEQ ID NO: 133 - RNA, 362 nt, Coleviroid
  MK477627.1 Coleus blumei viroid 3 isolate CbVd3_V_10_DOM, complete sequence
cggaucauuuucuugugguuccugugguacaccuccgaaggccgccuaggcgagaagagaguaaauagaggagagagcuuaccuggguucccuggcagcgcugcaacggaauucagggcguuggccccgauggaguuuccgaugagcaccggggagaagagacugucucgcagacuagaagucucuucuuuucugcuuugagacuucuucuccguaggacucuucggcucuucgcggguacccagcaaccugcucccacgcggcgauugcaaaggauccgguucgcucuccuuuucuuuuuuaccugcuucagaccgacgcgcccggggagugaaucaccugggaccgcgaaaagcgauccu

SEQ ID NO: 134 - RNA, 362 nt, Pospiviroid
  KY522737.1 Potato spindle tuber viroid isolate MT41ozon, complete genome
auccccggggaaaccuggagcgaacuggcaaaaaaggacgguggggagugcccagcggccgacaggaguaauucccgccgaaacaggguuuucacccuuccuuucuucggguguccuuccucgcgcccgcaggaccaccccucgcccccuuugcgcugucgcuucggcuacuacccgguggaaacaacugaagcucccgagaaccgcuuuuucucuaucuuacuugcuucggggcgaggguguuuagcccuuggaaccgcaguugguuccucggaacuaaacucgugguuccugugguucacaccugaccuccugagcagaaaagaaaaaagaagaaggcggcucggaggagcgcuucaggg

SEQ ID NO: 135 - RNA, 361 nt, Hostuviroid
  AF462154.1 Grapevine viroid clone Syrah complete genome
ugggcaccaacuagagguuccugugguacucaccgaaggcgcgaacguuggaaaagaaaaguagaaagcuggguaagacucaccuggcgacucgucgucgacgaaggguccucagcagagcaccggcagaggcgcuaugcaggaacgcuagggauccuucagcggaggacugaagaaacuccgguuucuucuuucacucuguagcuggaauccuuguugcgcuugcuggcgaaaccugcaggaagcuagcuggguccgcuagucgagcgacucgucccagcgucccaaccaguuuucuuuauccuauuuuuccucgcggacgcccggucguguuacccuagagcucccuguuuggaggccc

SEQ ID NO: 136 - RNA, 361 nt, Pospiviroid
  KP863711.1 Mexican papita viroid isolate chimera 3, complete sequence
cgggaucuuuuccuugugguuccugugguucacaccugaccuccagcccaggaaagaaaaaagaaaggcggcucggaggagcgcuucagggauccccggggaaaccuggagcgaacuggcaaaggagaggacggcuggggagucuuccagacaggaguaauccccgcugaaacaguguuuucacacuuccuuucuucggguuuccuuccucugagucgacacccucgcccgccuucucuugcgcugucgcuucggauacuacccgguggaaacaacugaagcucccgagaaccgcuuuuucucuaucuugcuggcgcaggggcgaggguggaaagcccuggaacccgcuggaugggucccu

SEQ ID NO: 137 - RNA, 361 nt, Pospiviroid
  GQ131572.1 Tomato chlorotic dwarf viroid isolate Mex9580, complete genome
cggaacuaaacucgugguuccugugguucacaccugaccuccugagcagaaaagaaaaaaagauaggcggcucggaggagcgcuucagggauccccggggaaaccuggagcgaacuggcaaaaggcggcagggagcuuguggaaggcgaaacaggaguaagcccgcgagaaacaggguuuucacccuuccuuucugacgcggcuuccuuccuuugcgcgccacucgaccccucgcccccuugcgcugucgcuucggcuacuacccgguggaaacaacugaagcucccgagaaccgcuuuuucucuauucuugcugcuuccggggcgaggguguuuagcccuuggaaccgcaguugguuccu

SEQ ID NO: 138 - RNA, 360 nt, Pospiviroid
  NC_001558.1 Tomato planta macho viroid, complete genome
cgggaucuuuuccuugugguuccugugguacacaccugaccuccugaccagaaaagaaaaaagaauugcggccaaaggagcgcuucagggauccccggggaaaccuggagcgaacuggcgaaggagucgcggcuggggagucucccagacaggaguaauccccgcugaaacaggguuuucacccuuccuuucuucggguuuccuuccucugcggucgacacccucgcccgcuucucuugcgcugucgcuucggagacuacccgguggaaacaacugaagcucccaagcgccgcuuuuucucuaucuugcuggcuccggggcgaggguggaaaacccuggaacccuucgaaaagggucccu

SEQ ID NO: 139 - RNA, 360 nt, Pospiviroid
  JQ975098.1 Tomato chlorotic dwarf viroid, complete genome
uuccucggaacuaaacucgugguuccugugguucacaccugaccuccugukcagaaaagaaaaaagauaggcggcucggaggagcgcuucagugauccccggggaaaccuggagcgaacuggcaaaaggcggcagggagcuuguggaaggcgaaacaggaguaaucccguguagaaacaggguuuucacccuuccuuucuucugcgguuuccuuccuuugcgcgccacucgaccccucgcccccuugcgcugucgcuucggcaacuacccgguggaaacaacugaagcucccuagaaccgcuuuuucucuaucuugcugcuaccggggcgaggguguuagcccuuggaaccgcaguugga

SEQ ID NO: 140 - RNA, 360 nt, Pospiviroid
  X95293.1 Tomato apical stunt viroid-S stem-loop RNA
cgggaucuuuccuugugguuccugugguacacaccugacccugcaggcaugaagaaaaaagaaggagcgggaaggaagaaguccuucagggauccccggggaaaccuggaggaagucgaggucgggggcucggaucuuccugguugagacagguguaacccagcugaaacaggguuuucacccuuccuuucuucugguuuccauccucucgccggaaggucuucggcccucgcccgaggcuucucucuggagacuacccgguggaaacaacugaagcuuccacuuccacgcucuuuuucuacuucuuucuugcucuccgggcgaggguggaaaacccuggaacccuucgaaagggucccu

SEQ ID NO: 141 - RNA, 359 nt, Pospiviroid
  HQ452399.1 Potato spindle tuber viroid isolate ca1a, complete genome
cggaacuaaacucgugguuccugugguccacaccugaccuccugagcauguaaagaaaaaagaauugcggcucggaggagcgcuucagggauccccggggaaaccuggagcgaacuggcaauaaggacgguggggagugccuuggucgaccugcguaauucccgccgaaacaggguuuucacccuuccuuucuugggguguccuuccccgcgcccggaggaccgccccucgcccccuuggcgcugucgcuucggcuacuacccgguggaaacaacugaagcucccgagagccgcuuuuucucuaucuuccuugcuuccggggcgaggguguuuagcccuuggaaccgcaguugguuccu

SEQ ID NO: 142 - RNA, 359 nt, Pospiviroid
  FJ797614.1 Potato spindle tuber viroid isolate 092009595, complete genome
cggaacuaaacucgugguuccugugguucacaccugaccuccuuuccagagaaagaaaaagauaggcggcucggaggagcgcuucagggauccccggggraaccuggagcgaacuggcaaaaggcgcgguggggagugccucgcggccgacaggaguaauuccugcugaaacaggguuuucacccuuccuuucuucagguuuccuuccucgcgcccgcaggaucacccccucgcccccuugcgcugucgcuucggcuacuacccgguggaaacaacugaagcucccgagaaccgcuuuuucucuaucuugcugguaucggggcgaggguguuuagcccuuggaaccgcaguugguuccu

SEQ ID NO: 143 - RNA, 359 nt, Pospiviroid
  AY222078.1 Columnea latent viroid isolate PSTVd*/CLVd, complete genome
cggaacuaaacucgugguuccugugguucacaccugaccucgugggcagaaaagaaaaaagaaggcggcucggaggagcggucucaggagccccggggcaacucagaccgagcggggucucguggucgagggcguagccccuguucagacaggaguaaucccagcugaaacaggguuuucacccuuccuuucuucugguuuccuuccucugcuucagcggccucgcccggaaccucuugaccagcgcaggugcugacgcgaccgguggcaucaccgaguugcucccgagaaccgcuuuuucucuaucuuacuugccuccgggcgaggguguuuagcccuuggaaccgcaguugguuccu

SEQ ID NO: 144 - RNA, 358 nt, Apscaviroid
  NC_021720.1 Persimmon viroid 2 genomic RNA, complete genome
ggagaaaacuccgugugguuccuguguggcaccccucccgcuacgggcuuuaaaaagaaagcagggaaggguacuuaccugucgucgucgacgaaggcugguaagcagagcccguugaagcuccccgugacgagcgucgucggauggccuccgagacgagcgauggacaguggagcucugcucucaccagucgcguguugacucucgcgccuacccgaacacggcuguucugagggcgcugagggccgagcuucccgccgcuagucgagcggaccacugguaguucccccgaccugccuauuuuuguagccuagggagaguggagucaccaggguaaaacacgauugguguuuucccc

SEQ ID NO: 145 - RNA, 358 nt, Pospiviroid
  MK330995.1 Potato spindle tuber viroid isolate 21809902-95 sequence
auccccggggaaaccuggagcgaacuggcaaaaggcgcgguggggagugccucgcggccaacaggaguaauuccugcugaaacaggguuuucacccuuccuuucuucagguuuccuuccuugcgcccgcaggaucaccccucgcccccuugcgcugucgcuucggcuacuacccgguggaaacaacugaagcucccgagaaccgcuuuuucucuaucuugcugguaucggggcgaggguguuuagcccuuggaaccgcaguugguuccucggaacuaaacucgugguuccugugguucacaccugaccuccuuuccagaaaagaaaaaagauaggcggcucggaggagcgcuucaggg

SEQ ID NO: 146 - RNA, 358 nt, Pospiviroid
  KJ857498.1 Potato spindle tuber viroid isolate CVN212, complete genome
gcucggaggagcgcuucagggauccccggggaaaccuggagcgaacuggcaaaaggcgcgguggggagugccucgcggccgacaggaguaauuccugcugaaacaggguuuucacccuuccuuucuucagguuuccuuccucgcgcccgcaggaucaccccucgcccccuugcgcugucgcuucggcuacuacccgguggaaacaacugaagaucccgagaaccgcuuuuucucuaucuugcugguaccggggcgaggguguuuagcccuuggaaccgcaguugguuccucggaacuaaacucgugguuccugugguucacaccugaccuccuguccagaaaagaaaaaagauaggcg

SEQ ID NO: 147 - RNA, 356 nt, Pospiviroid
  NC_002015.1 Chrysanthemum stunt viroid, complete genome
cgggacuuacuugugguuccuguggugcacuccugacccugcugcuauagcaaagaaaaagaaaugaggcgaagaaguccuucagggauccccggggaaaccuggaggaaguccgacgagaucgcgguuggggcuuaggaccccacuccugcgagacaggaguaauccuaaacaggguuuucacccuuccuuuaguuuccuuccucuccuggagaggucuucugcccuagcccggucuucgaagcuuccuuuggcuacuacccgguggaaacaacugaagcuucaacgccuuuuuuuccaaccuucuuuagcaccgggccagggaguuagcccuuggaaccuuaguauuguucccu

SEQ ID NO: 148 - RNA, 356 nt, Pospiviroid
  LC192496.1 Chrysanthemum stunt viroid genomic RNA, complete genome, isolate: US2.111
ggggaaaccuggaggaaguccgacgagaucgcggcuggggcuuaggaccccacuccugcgagacaggaguaaaccuaaacaggguuuucacccuuccuuuaguguccuuccucuccuggagcggucuucugcccuagcccggucuucgaagcuuccuuuggcuacuacccgguggaaacaacugaagcuucaacgccuuuuuuuccauaucuucuuuagcaccgggcuagggaguaagcccguggaaccuuaguuuuguucccucgggacuuacuugugguuccuguggugcacuccugacccugcugcuauacaagaaaaagaaauagaggcgaagaaguccuucagggaucccc

SEQ ID NO: 149 - RNA, 356 nt, Pospiviroid
  LC192474.1 Chrysanthemum stunt viroid genomic RNA, complete genome, isolate: US2.51
ggggaaaccuggaggaaguccgacgagaucgcggcuggggcuuaggaccccacuccugcgagacaggaguaauccuaaacaggguuuucacccuuccuuuaguuuccuuccucuccuggagaggucuucugcccuagcccggucuucgaagcuuccuuuggcuacuacccgguggaaacaacugaagcuucaacgccuuuuuuuuccuaucuucuuuagcaccgggcuagggaguaagcccguggaaccuuaguuuuguucccuccggacuuacuuguggguccuguguuuggacuccugacccugcugcuauacaauaaaaagaaauguuguuuauauguccuucagugaucccc

SEQ ID NO: 150 - RNA, 355 nt, Pospiviroid
  KX096422.1 Chrysanthemum stunt viroid isolate Ford-26, complete sequence
aaagaaaugaggcgaagaaguccuucagggauccccagggaaaccuggaggaaguccgacgagaucgcggcuggggcuuaggaccccacuccugcgagacaggaguaauccuaaacaggguuuucacccuuccuuuaguuuccuuccucuccuggagaggucuucugcccuagcccggucuucgaagcuuccuuuggcuacuacccgguggaaacaacugaagcuucaacgccuuuuuuuccaaucuucuuuagcaccgggcuagggaguaagcccguggaaccuuaguuuuguucccucgggacuuacuugugguuccuguggugcacuccugacccugcugcuuugaaagaaa

SEQ ID NO: 151 - RNA, 354 nt, Apscaviroid
  MF521431.2 Apple chlorotic fruit spot viroid, complete genome
ggguucuucaacuuccugugguuccuguggugacaccuccgaaggccgccuagcugaaagaaaaaggaaaaaagaucgggcggagagggucgagggacucgucgucgacgaagggccucucguguccaguucucgccugcuccuccagcggguacccuagguaaaauuuucuaaagaaauuucuuaaaaaaguucuaggaauaccuguagcuggaagcuggcugagauuuggcucgagcaguccgcuagucgcgcggacuugucuccuuuccccaaaccgccuuuuucucuauccuccucuugaagcgacgcgcccgggagugaagccuuaggaccgcggagcuguugagccuc

SEQ ID NO: 152 - RNA, 354 nt, Pelamoviroid
  JN377886.1 Peach latent mosaic viroid clone C40-A349-g12.83, complete sequence
cucaaaaguuucguugcauuucaucaacucaucagugggcuuagcccagacuuuugagagaauaaacaccucucagccccuccaccuuggggugcccuauccgagcacugcaguuucgguagaaaggcuaagcaccucgcaaugagguaaggugggacuuuuccuucuggaaccaagcgguugguuccgaggggggugugauccagguaccgccguagaaacuggauuacgacgucuacccgggauucaaacccgguccccuccagaagugaauucuggaugaagagucugugcuaagcacacugacgagucucugaaaugagacgaaacucuucagaaacuuugguuugguuu

SEQ ID NO: 153 - RNA, 354 nt, Pospiviroid
  KP262537.1 Chrysanthemum stunt viroid isolate CSVd-4, complete genome
cgggacuuacuugugguuccuguggcgcacuccugacccugcugcuuugcaagaaaaagaaaugacccgaagaaguccuucagggauccccggggaaaccuggaggaaguccgacgagaacgcggcuggggcuuagggccccacuccugcgagacaggaguaauccuaaacaggguuuucacccuuccucuaguuuccuuccucuccuggagaggucuucugcccuagcccgguccucgaagcuuccuuuggcaacuacccgguggaaacaacugaagcuucaacgccuuuuuuuccaaucuucuuuagcgccgggcuagggaguaagcccguggaaccuuugguuuguucccu

SEQ ID NO: 154 - RNA, 354 nt, Pospiviroid
  KP262535.1 Chrysanthemum stunt viroid isolate CSVd-2, complete genome
cgggacuuacuugugguuccuguggugcacuccugacccagcugcuuugaaagaaaaagaaaugagccgaggaaguccuucagggaucccaggggaaaccuggaggaaguccgacgagaucgcggcuggggcuuaggaccccacuccugcgagacaggaguaguccuaaacaggguuuucgcccuuccuuuaguuuccuuccucuccuggagaggucuucugcccuagcucgguucucgaagcuuccuuuggcuacuacccgguggaaacaacugaagcuucaacgccuuuuuucccaaucuucuuuggcaccgggcuagggaguaagcccguggaaccaugguuuuguucccu

SEQ ID NO: 155 - RNA, 352 nt, Apscaviroid
  KY473930.1 Citrus dwarfing viroid isolate En-CT-Ver, complete genome
ucuuucgucgacgacgacagguacuucgaaagaagaauccauucacaguaaauccucgucaguuucgucgaugacgacagguaaguuucccuuucccucucugcguuuuauuuugcaagggggugugccccacaggaaccacacggaguuuccuccggggaaacaccaacguguuuuacccuggaggcccaauccccuucuagccuagauaaaaacagauuaggauuggacaacucacucaguuguccgcucgacuagcgggagcuagaagggucugccggaguugcuggaucuuuacgacuaccggagacucuccgucuuuacuccacucggcgucaccacuuagcugcca

SEQ ID NO: 156 - RNA, 351 nt, Pelamoviroid
  DQ680797.1 Peach latent mosaic viroid variant 94.2, complete genome
cucauaaguuucgccgcaucucagcggcucaucagugggcuaagcccagacuuaugagagauuaagaccucucagccccuccaccuuggggugcccuauucggagcacugcaguucccgauagaaaggcuaagcaccucgcaaugagguaaggugggacuuuuccuucgggaaccaagcgguugguuccgaggggggugugauccagguaccgccguagaaacuggauuacgacgucuacccgggauucaaacccgguccccuccagaagugauucuggaagaagagucugcgcuaagcgcacugaugagucucugagaugagacgaaacucuucaguuuuccucagaaaa

SEQ ID NO: 157 - RNA, 351 nt, Pelamoviroid
  DQ222090.1 Peach latent mosaic viroid isolate PC-P1.148.73, complete genome
cucaaaaguuucgccgcauuucagcggcucaucagugggcuuagcccagacuuuugagagauuaacgaccucucagccccuccaccuuggggugcccuauccgagcacugcaguuucgguagaaaggcuaagcaccucgcaaugagguaaggugggacuuuuccuucugaaaccaagcgguugguuccgaggggggugugauccagguaccgccguagaaacuggauuacgacgucuacccggaauucaaucccgguccccuccagaagugaauucuggaagaagagucucugcgcuaagcgcacugaugagucucugaaaugagacgaaacucuucaggaacuuuuguua

SEQ ID NO: 158 - RNA, 351 nt, Pelamoviroid
  AJ550910.1 Peach latent mosaic viroid complete genome, isolate PC-C37
uucauaaguuucgucgcaucgcagcgacucaucagugggcuaagcccagacuuaugagagaauuaacaccucucagccccuccaccuuggggugcccuagucggacacugcaguuccgauggaaaggcuaagcacgucgcaaugacguaaggugggacuuuuccuucugggaccaagcgguugguuccgaggggggugugauccagguaccgccguagaaacuggauuacgacgucuacccgggauucaaacccgguccccuccagaagugauucuggauugaagaguccugcgcuuagcgcacugacgagucucugagaugagacgaaacucuucgagagcuuauaguuc

SEQ ID NO: 159 - RNA, 351 nt, Pospiviroid
  NC_027432.1 Portulaca latent viroid isolate Vd21, complete genome
ugugguuccuaugguugcaccccugaccugcuaccugcaaaagaaaaaagaugggcaggcgagcacaacgcuucagggauccccggggaaaccuggagcgaccucggcaggaaggcuuggcgguaggugaagaaggagcgagcgccguaacgaccugagaacaggagaaacaggagcucgacuccuuccuuucuucucucaucgggcaggugccgcuucccuccacccucgccggucuucugcgcuggcgcucuggcuacuacccgguggauacaacugacgcuuguacuccgcccugccuuuuucuacuucuucgcuggagcggcgagggugggcuucuaaggaaacccu

SEQ ID NO: 160 - RNA, 350 nt, Pelamoviroid
  JN377883.1 Peach latent mosaic viroid clone C40-stem-g12, complete sequence
cccaaaaguuucguugcauuucagcaacucaucagugggcuuagcccagacuuuugagagaauaaauaccucucagccucuccaccuuggggugcccuauccgaacacugcaguuucgguagaaaggcuaagaaccucgcaaugagguaaggugggacuuuuccuucuggaaccaagcgguugguuccgaggggggugugauccagguaccgccguagaaacuggauuacgacgucuacccgggauucaaacccgguccccuccagaagugaauucuggaugaagagucugugcuaagcacacugacgagucucugaaaugagacgaaacucuucaauucgucuaacgaa

SEQ ID NO: 161 - RNA, 350 nt, Pelamoviroid
  GQ872137.1 Peach latent mosaic viroid isolate Laura.4, complete genome
cucauaaguuucgccuuguuucaaaagcucaucagugggcuaagcccagacuuaugagaguuuaacaccucucagccccuccaccuuggggugcccuauucggagcacugcaguucccgauagaaaggcuaagcaccucgcaaugacguaaggugggacuuuuccuucuggaaccaagcgguugguuccgaggggggugugauccagguaccgccguagaaacuggauuacgacgucuacccgggauucaaacccgguccccuccagaagugauucuggaugaagagucugcgcuuagcgcacugaugagucuuugaaauaagacgaaacucuucgaauuucguuuggaa

SEQ ID NO: 162 - RNA, 350 nt, Pelamoviroid
  DQ680799.1 Peach latent mosaic viroid variant 88.1, complete genome
cucaaaaguuucgucgcaucucagcgacucaucagugggcuuagcccagacuuaugagagauuaaagaccucucagccccuccaccuuggggugcccuauucggagcauugcaguucccgauagaaaggcuaagcaccucgcaaugacguaaggugggacuuuuccuucgggaaccaagcgguugguuccgaggggggugugacccagguaccgccguagaaacuggguuacgacgucuacccggaauucaaucccgguccccuccagaagugaauucuggaagaagagucugugcuaagcacacugacgagucucugagaugagacgaaacucuucaguuccauugguu

SEQ ID NO: 163 - RNA, 350 nt, Pelamoviroid
  DQ222055.1 Peach latent mosaic viroid PC-P2.33, complete genome
cucaaaaguuucgccuuaucucaaaggcucaucagugggcuuagcccagacuuuugagagauuaaagaccucucagccccuccaccuuggggugcccuauccgaagcacugcaguuucgguagaaaggcuaaggaccucgcaaugagguaaggugggacuuuuccuucuggaaccaagcgguugguuccgaggggggugugauccagguaccgccguagaaacuggguuacgacgucuacccgggauucaaacccgguccccuccagaagugaauucuggaugaagagucugugcuaagcacacugacgagucucugagaugagacgaaacucuucaagagcuuuuguuc

SEQ ID NO: 164 - RNA, 350 nt, Pelamoviroid
  DQ222053.1 Peach latent mosaic viroid PC-P1.151, complete genome
cucaaaaguuucgccgcaucucagcggcucaucagugggcuuagcccagacuuuugagaguauaaacaccucucagccccuccaccuuggggugcccuauucgggacacugcaguuccgauagaaaggcuaagaacgucgcaaugacguaaggugggacuuuuccuucuggaaccaagcgguugguuccgaggggggugugauccagguaccgccguagaaacuggauuacgacgucuacccgggauucaaacccgguccccuccagaagugaauucuggaugaagagucugcgcuaagcgcacugaugagucucugagaugagacgaaacucuucagaaacuuuuguuu

SEQ ID NO: 165 - RNA, 350 nt, Pelamoviroid
  DQ222074.1 Peach latent mosaic viroid isolate PC-P1.142.12, complete genome
cucauaaguuucgucgcaucucagcggcucaucagugggcuuagcccagacuuaugagaguauaagaccucucagccccuccaccuuggggugcccuaaccggacacugcaguuucggaagaaagggcuaagcaccucgcaaugagguaaggugggacuuuuccuucuggaaccaagcgguugguuccgaggggggugugauucagguaccgccguagaaacugaauuacgacgucuacccgggauucaaacccgguccccuccagaagugauucuggaugaagagucugcgcuaagcgcacugaugagucucugaaaugagacgaaacucuucgguaauagaaguauua

SEQ ID NO: 166 - RNA, 350 nt, Pospiviroid
  JF446895.1 Pepper chat fruit viroid isolate NK10-6c4, complete genome
ccggauucuucuaagggugccuguggugccucccccgaagcccgcuuagggaaagagaaaggggaagcaagcaucuccuguucagggauccccggggaaaccuggacagaccgggcggagaagcgcacgagcgggaccgucuucugacaggaguaauccccgcagaaacauggguuuucacccuuccuuucuucggguuuccuuccucagucgaccgguccgcgucggccuucucgcgcacugcuguccggcuacuacccgguggauacaacugacagaggugcuuuuucuuccacccgacuucuaccgacgcggccgggagugaagcuacccgggacccgagaggaucu

SEQ ID NO: 167 - RNA, 349 nt, Pelamoviroid
  GQ872138.1 Peach latent mosaic viroid isolate Luara.5, complete genome
cucauaaguuucgccgugucucaacggcucaucagugggcuaagcccagacuuaugagagaguaagaccucucagccccuccaccuuggagugcccuauucggagcacugcaguucccgauagaaagguuaagcacuucgcaaugacguaaggugggacuuuuccuucgggaaccaagcgguugguuccgaggggggugugauccagguaccgccguagaaacuggauuacgacgucuacccgggauucaaacccgguccccuccagaagugauucuggaagaagagucgcgcuuagcgcacugaugggucuuugagauaagacgaaacucuucgacuuucgaaagaaa

SEQ ID NO: 168 - RNA, 348 nt, Pospiviroid
  JF446909.1 Pepper chat fruit viroid isolate LP2-5c4, complete genome
ccggauucuucuaagggugccuguggugccucccccgaagcccgcuuagggaaaaagaaaggggaagcaagcaucuccuguucagggauccccggggaaaccugaacggaucgggcggagaaguuccgugcggguccgucuucuggcaggaguaaucccagcugaaacaggguuuucacccuuccuuucuucggguuuccuuccucagucgaccggaccgcgucggccuucucgcgcacuucuguucggagacuacccgguggauacaacugacagaggugcuuuuucuuccacccugcuucuaccgacgcggccgggagugaagcuacccgggacccgagaggaucu

SEQ ID NO: 169 - RNA, 343 nt, Coleviroid
  FJ615419.1 Coleus blumei viroid 6 clone 2, complete genome
cggaucauuuucuugugguuccugugguacaccuccgaaggccgccuaggcgagaagagaguaaauagaggagagagcuuaccuggguucccuggcagcgcugcaacggaauucagggcacggagucggugccagaucgugcggcuucggcgccaguucgugcgagagcuucgcucgcgaacucuucguggaacggccgucgcucguggaucggccgucuuccggguuccugcucccacgcagcgauugcaaaggauccgguucgcucuccuuuucuaauuuuaccugcuucagaccgacgcgcccggggagugaaucaccugggaccgcgaaaagcgauccu

SEQ ID NO: 170 - RNA, 342 nt, Hostuviroid
  MK496633.1 Dahlia latent viroid isolate DLV/CU, complete sequence
caggucuucuaaggguuccuguggugccucccuccaaggccgcguagggaaagaaaaagugaaagaaaguguaccugaagagagagagacucucugaggagccccggggcaacuccgagagugcugcggcaggaggagccgggggcggagguugucucgcucuugagagcuccgcuccuuguagcuuugagacuaccgcccuuuugcuuccuucucgcuggcugacucgaggacgcgaccggugguacaccaagaggucuccaccuccuaggguacuuuuuucuaacaccgauuccguaccgagcgccggagagugaagcgcccagggcuccaaagaagccc

SEQ ID NO: 171 - RNA, 341 nt, Pelamoviroid
  KJ754183.1 Peach latent mosaic viroid isolate SDP2, complete sequence
uaacugcagugcuccgaauagggcaccccaagguggaggggcugagaggucaucacucucucauaagucugggcuuagcccacugaugagccguugagauacggcgaaacuuauggaugagaagaguuucgucucauuucagagacucaucagugugcuaagcacagacucuucuuccagaaucacuucuggaggggaccggguuugaaucccggguagacgucguaauccaguuccuacggcgguaccuggaucacaccccccucggaaccaaccgcuugguucccgaaggaaaagucccaccuuaccucauugcgaggugcuuagccuuucuaucggga

SEQ ID NO: 172 - RNA, 341 nt, Pelamoviroid
  KF870319.1 Peach latent mosaic viroid clone 1893_5_341 clone-lib P3, complete sequence
cccauaaguuucgucgcaucccagcgacucaucagugggcuaagcccagacuuaugagagagugguuaccucucagccccuccaccuuggggugcccuauucggagcacugcaguucccgauagaaaggcuaaggcacuuacgccaaugaaguaaggugggacuuuucccuaggguuugagcggucgaacccagggggagugugauccagguaccgccguagaaacuggauuacgacgccuacccgggauucaaacccggucccccucagaggugacucugagugaaaggucugugcuuagcacacugaugaguuccugaaauggaacgaaaccuuucuaa

SEQ ID NO: 173 - RNA, 341 nt, Pelamoviroid
  DQ680798.1 Peach latent mosaic viroid variant 235.2, complete genome
cucauaaguuucgccgcaucucagcggcucaucaguggggacuuaugagagaauuaacaccucucagccccuccaccuuggggugcccuauucggagcacugcaguucccgauagaaaggcuaagcaccucgcaaugacguaaggugggacuuuccuuccgggaccaagcgguuggucccgaggggggugugacccagguaccgccguagaaacuggguuacgacgucuacccgggauucaaacccgguccccuccagaagugauucuggauugagagucugcgcuaagcgcacugaugagucucugagaugagacgaaacucucgaacuaucgaaagaua

SEQ ID NO: 174 - RNA, 341 nt, Pelamoviroid
  DQ680718.1 Peach latent mosaic viroid variant 278.3, complete genome
cccaaaaguuucgccguaucucaacggcucaucagugggcuaagcccagacuuuugagagaguuggucaccucucagccccuccaccuuggagugcccuauucggagcacugcaguucccgauagcaaaggcuaagcaccucgcaaugagguaaggugggacuuuuccuucgggaaccaagcgguugguuccgaggggggugugauccagguaccgccguagaaacuggauuacgacgucuacccgggauucaaacccgguccccuccagaagugauucuggaagaagagucugugcucagcacacugaugagucucugaaaugagacgaaacucuucaga

SEQ ID NO: 175 - RNA, 340 nt, Apscaviroid
  FJ974062.1 Apple scar skin viroid clone Lynkestis-1, complete genome
gguaaacaccgugcgguuccugugguucgccccgccaacgcagauagauaaagaaaacgaggagaagaaggaacucaccugucgucgucaacgaaggccaaucacuagugcggccaaaggagcugccagcacuaagccggacggcgcccucgcaccaguuccgcuguggguucgccuacaagaacguacgguguugaggcccuguccgccgcugcgcugccaccuacucccgcgccgcuagucgagcggacuccggguggagcccccuguucucucacgcucuuuuucuuugacgcagcggcggguggguucccaggguaaaacacaauagguguuuccc

SEQ ID NO: 176 - RNA, 340 nt, Pelamoviroid
  MG788242.1 Peach latent mosaic viroid clone D4, complete genome
cacauaaguuucgccguaucucaacggcucaucagugggcuuagcccagacuuaugagagaguugugaccucucagccccuccaccuuggggugcccuagucggacacugcaguuccgauggaaaggcuaagcaccucgcaaugagguaaggugggacuuuuccuucuggaaccaagcgguugguuccgaggggggugugauccagguaccgccguagaaacuggauuacgacgucuacccgggauuccaaacccgguccccuccagaagugaauuacuggaugaagagucugugcuaagcacacugacgagucucugaaaugagacgaaacucuucuca

SEQ ID NO: 177 - RNA, 340 nt, Pelamoviroid
  MF574160.1 Peach latent mosaic viroid isolate MH35, complete genome
cccauaaguuucgccguaucucaacggcucgucugugggcuuagcccauacuuaugguagaguugaucaccucucauccccuccaccuuggggugcccuaucccagcacugcauuuucgguagaaaggcuaagaaccuccccaugaggcaaggugugacuuuuccuucuggaaccuagcggcugguuccgaggggggugugauccacguaccgccgcuacaaacuggguuacgaccccuuuaagggauucaaacccgguuccccuccagaagugauucuggaugaagagucugugcuacgcacacugacgagucucugaaaugauacuaaacucuucuga

SEQ ID NO: 178 - RNA, 340 nt, Pelamoviroid
  MF574156.1 Peach latent mosaic viroid isolate BH5, complete genome
uccauaaguuucgccguaucucaacggcucgucagugggcuuagcccagacuuaugggagaguugaucaccucucagccccuccaccuuggggugcccuauccgagcacugcaguuucgguagaaaggcuaagaaccucgcaaugagguaaggugggacuuuuccuucuggaaccaagcgguugguuccgaggggggugugauccaguuaccgccguagaaacuggguuacgacguccuacccgggauuccaaacccgguccccuccagaagugauucuggaugaagagucugugcuaagcacacugacgagucucugaaaugagacgaaacucuucuga

SEQ ID NO: 179 - RNA, 340 nt, Pelamoviroid
  KY355321.1 Peach latent mosaic viroid isolate PLMVd-JT-P1-24, complete genome
cucaaaaguuucgucgcaucccagcgacucaucagugggcuuagcccagacuuuugagagaauuagucaccucucagccccuccaccuuggagugcccuauucggagcacugcaguucccgauagaaaggcuaagcaccucgcaaugacguaaggugggacuuuucccuugaguucaagcgguuggacucagaggggagugugauccugguaccgccguagaaacaggauuacgacgccuuaccgagauucaaacucggccccucucagaggugacucugagcgaaaggucugugcuaagcacacugaugaguuccugaaauggaacgaaaccuuucuga

SEQ ID NO: 180 - RNA, 340 nt, Pelamoviroid
  KY355281.1 Peach latent mosaic viroid isolate PLMVd-JH-P1-25, complete genome
cccauaaguuucgucgcaucccagcgacucgucagugggcuuagcccagacuuaugagagaagugguuaccucucagccccuccaccuuggagugcccuauucggagcacugcaguucccgauagaaaggcuaagcaccucgcaaugacguaaggugggacuuuucccuugaguuugaguggucgaacucagaggggagugugauccugguaccgccguagaaacaggauuacgacgucuuaccgagauucaaacucggccccucucagaggugacccugagcgaaaggucugugcuaagcacacugacgaguuccugcaauggaacgaaaccuuucuaa

SEQ ID NO: 181 - RNA, 340 nt, Pelamoviroid
  DQ680801.1 Peach latent mosaic viroid variant 235.4, complete genome
accauaaguuucgccgcgucucagcggcucaucagugggcuuagcccagacuuaugagagaguaaagaccucucagccccuccaccuuggggugcccuauucggagcacugcaguucccgauagaaaggcuaagcaccucgcaaugacguaaggugggacuuuccuuccgggaccaagcgguuggucccgaggggggugugacucagguaccgccguagaaacugaguuacgacgucuacccgggauucaaacccgguccccuccagaagugauucuggauugagagucugugcuaagcacacugaugagucucugagaugagacgaaacucucugguuc

SEQ ID NO: 182 - RNA, 340 nt, Pelamoviroid
  DQ680779.1 Peach latent mosaic viroid variant 133′.1, complete genome
cccauaaguuucgccguaucucaacggcucaucagugggcuuagcccagacuuaugagaguaauggucaccucucagccccuccaccuuggagugcccuauucggagcacugcaguucccgauagaaaggcuaagcaccucgcuaugacguaaggugggacuuuuccuucuggaaccuagcgguagguuccgaggggggugugacucagguaccgccguagaaacugaguuacgacgucuacccgggauucaaacccgguccccuccagaagugauucuggaugaagagucugugcuaagcacacugaugagucucugaaaugaggcgaaacucuuuuga

SEQ ID NO: 183 - RNA, 339 nt, Pelamoviroid
  KY355220.1 Peach latent mosaic viroid isolate PLMVd-CH-P1-19, complete genome
ucaaaaguuucgucgcaucccagcgacucaucagugggcuuagcccagacuuuugagaguagugaugaccucucagccccuccaccuuggagugcccuauucggagcacugcaguucccgauagaaaggcuaagcaccucgcaaugacguaaggugggacuuuucccucggguuugagcggucgaacccagaggggagugugauccugguaccgccguagaaacaggauuacgacgucuuaccgggauuccaacccggccccucucagaggugacucugagcgaaaggucugugcuaagcacacugaugaguuccugaaauggaacgaaaccuuucuca

SEQ ID NO: 184 - RNA, 339 nt, Pelamoviroid
  KY355176.1 Peach latent mosaic viroid isolate PLMVd-BC-P1-19, complete genome
cucaaaaguuucgucgcaucccagcgacucgucagugggcuuugcccagacuuuugagagaaauggucaccucucagccccuccaccuuggagugcccuauucggagcacugcaguucccgauagaaaggcuaagcaccucgcaaugacguaaggugggacuuuucccuacaguucgagcggucgaacuagaggggaguguaaccuugguaccgccguagaaacaggguuacgacgucuuaccgagauuccaacucggccccucucagaggugacucugagcgaaaggucugugcaaagcacacugacgaguuccugaaauggaacgaaaccuuuuuga

SEQ ID NO: 185 - RNA, 339 nt, Pelamoviroid
  KY310588.1 Peach latent mosaic viroid isolate JH, complete genome
uucauaaguuucgucgcaucccagcgacucaucagugggcuuagcccagacuuaugagagaagugguuaccucucagccccuccaccuuggggugcccuauucgaggcacugcagucucgauagaaaggcuaagaaccucgcaaugagguaaggugggacuuuuccuucuggaaccaagcgguugguuccgaggggggugugauccagguaccgccguagaaacuggauuacgacgucuacccgggauucaaacccgguccccuccagaagugauucuggaugaagagucugugcuuagcacacugaugagucucugaaaugagacgaaacucuucuca

SEQ ID NO: 186 - RNA, 339 nt, Pelamoviroid
  KU048793.1 Peach latent mosaic viroid isolate plum1.3, complete genome
cccauaaguuucgccguaucucaacggcucgucagugggcuuagcccagacuuaugagagagugguuaccucucagcccuuccaccuuagagugcccuauucggagcacugcaguucccgauagaaaggcuaagcaccucgcaaugagguaagguggggcuuuuccuucaggaaccaagcgguuggguccgaggggggugugacucagguaccgccguagaaacugaguuacgacgucuacccgggauucaaacccgguccccuccagaagugauucuggaagaagagucugugcuaagcacacugacgagucucugaaaugagacgaaacucuucuaa

SEQ ID NO: 187 - RNA, 339 nt, Pelamoviroid
  KF870034.1 Peach latent mosaic viroid clone 1593_5_339 clone-lib P3, complete sequence
cccauaaguuucgucgcaucccagcgacucaucagugggcuaagcccagacuuaugagagagugguuaccucucagccccuccaccuuggggugcccuauucggagcacugcaguucccgauagaaaggcuaagaaccucgcaaugagguaaggugggacuuuuccuucgggaaccaagcgguugguuccgaggggggugugauccagguaccgccguagaaacuggauuacgacgccuacccgggauucaaacccggucccccucagaggugacucugagugaaaggucugugcuuagcacacugacgaguuccugaaauggaacgaaaccuuucuaa

SEQ ID NO: 188 - RNA, 339 nt, Pelamoviroid
  JX480364.1 Peach latent mosaic viroid clone 966_9_339 clone-lib P3, complete sequence
acucaucagugggcuaagcccagacuuaugagagagugguuaccucucagccccuccaccuuggggugcccuauucggagcacugcaguucccgauagaaaggcuaagcacuucgcaaugaaguaaggugggacuuuucccuaggguuugagcggucgaacccagggggagugugauccagguaccgccguagaaacuggauuacgacgccuacccgggauucaaacccggucccccucagaggugacucugagugaaaggucugugcuuagcacacugaugaguuccugaaauggaacgaaaccuuucuaacccacuaaguuucgucgcaucccagcg

SEQ ID NO: 189 - RNA, 339 nt, Pelamoviroid
  GU825983.1 Peach latent mosaic viroid clone PLMVd-2tun, complete genome
cauaaguuucgccguaucucaacggcucaucagugggcuaagcccagacuuaugagagagugaugaccucucagccccuccaccuuggggugcccuaaucgaggcacugcagcuucgauggaaaggcuaagaaccucgcaaugagguaaggugggacuuuuccuucuggaaccaagcgguugguuccgagggggggugugauccagguaccgccguagaaacuggauuacgacgucuacccgggauucaaacccgguccccuccagaagugauucuggaagaagagucagugcuuagcacacugaugagucucugaaaugagacgaaacucuucucauc

SEQ ID NO: 190 - RNA, 339 nt, Pelamoviroid
  DQ839564.1 Peach latent mosaic viroid clone Eg18, complete genome
aucaaaaguuucgccguaucucaacggcucaucagugggcuuagcccagacuuuugggagaauaaacaccucucagccccuccaccuuggggugcccuauucggagcacugcaguucccgauagaaaggcuaagcaccucgcaaugagguaaggugggacuguuccuucuggagccaagcgguuggcuccgaggggggugugauccagguaccgccguagaaacuggauuacgacgucuacccgggauucaaacccgguccccuccagaagugauucuggauugaagagucugugcuaagcacacugaugagucucugagaugagacgaaacucuucuc

SEQ ID NO: 191 - RNA, 339 nt, Pelamoviroid
  DQ680806.1 Peach latent mosaic viroid variant 387.1, complete genome
aucaaaaguuucgucacauuucagugacucaucagugggcuuagcccagacuuuugggagaauaaacaccucucagccccuccaccuuggggugcccuauucggagcacugcaguucccgauagaaaggcuaagcaccucgcaaugagguaaggugggacuuuuccuucugagaccaagcgguuggucucgaggggggugugacccagguaccgccguagaaacuggguuacgacgucuacccgggauucaaacccgguccccuccagaagugaauucuggaugaagagucugugcuaagcacacugacgagucccugagaugggacgaaacucuucuc

SEQ ID NO: 192 - RNA, 339 nt, Pelamoviroid
  DQ680789.1 Peach latent mosaic viroid variant 330.1, complete genome
uccauaaguuucgccguaucucaacggcucaucagugggcuuagcccagacuuaugagagagugaugaccucucagccccuccaccuuggagugcccuauucggagcacugcaguucccgauagaaaggcuaagcaccucgcuaugagguaaggugggacuuuuccuucgggagccaagcgguuggcuccgaggggggugugacccagguaccgccguagaaacuggguuacgacgucuacccggaauucaaucccgguccccuccagaagugauucuggaagaagagucugcgcuaagcgcacugaugagucucugaaaugagacgaaacucuucuca

SEQ ID NO: 193 - RNA, 339 nt, Pelamoviroid
  DQ680783.1 Peach latent mosaic viroid variant 369.4, complete genome
caaauuaguuucgccguaucucaacggcucaucagugggcuuugcccagacuaauuugagaguagucaccucucagcccuuccaccuuagagugcccuauucggagcacugcaguucccgauagaaaggcuaagcaccucgcaaugagguaaggugggacuuuuccuucuggaaccaagcgguugguuccgaggggggugugacccagguaccgccguagaaacuggguuacgacgucuacccgggauucaaacccgguccccuccagaagugauucuggaugaagagucugcgcaaagcgcacugaugagucucugaaaugagacgaaacucuuuuga

SEQ ID NO: 194 - RNA, 339 nt, Pelamoviroid
  DQ680774.1 Peach latent mosaic viroid variant 369.1, complete genome
aacaaaaguuucgccguaucucaacggcucaucagugggcuuagcccagacuuuugagagaauaagcaccucucagccccuccaccuuggagugcccuauucggagcacugcaguucccgauagaaaggcuaagcaccucgcaaugagguaaggugggacuuuuccuucuggaaccaagcgguugguuccgaggggggugugacucagguaccgccguagaaacugaguuacgacgucuacccgggauucaaacccgguccccuccagaagugaauucuggaugaagagucugcgcuaagcgcacugacgaguccuugagauaggacgaaacucuucuc

SEQ ID NO: 195 - RNA, 339 nt, Pelamoviroid
  AF170504.1 Peach latent mosaic viroid variant #11 from Prunus persica Redhaven cultivar, complete sequence
gccaaaaguuucgccguaucucaacggcucaucagugggcuuagcccagacuuuugagagaaguagcgaccucucagccccuccaccuuggggugcccuauucggagcacugcaguucccgauagaaaggcuaagcaccucgcagugagguaaggugggacuuuuccuucuggaaccaagcgguugguuccgaggggggugugauccagguaccgccguagaaacuggauuacgacgucuacccgggauucaaacccgguccccuccagaagugauucuggaagauaagucugugcuaagcacacugaugagucucugaaaugagacgaaacuuaucuc

SEQ ID NO: 196 - RNA, 339 nt, Pospiviroid
  MH995516.1 Citrus exocortis viroid isolate VC VIV 3 M20, partial genome
ggucgggggggaucagcagcaucggucgccgcggaucacaggcguccagcggagaaacaggagcuggucuccuuccgugcgcugcuggcuccacauccgaucgucgcugaagcuucgcgcccccucgccaggagguucucucuggcuacuacccgguggauacaacugaagcuucaaccccaaaccgcuuuucuuauaucuucacugcucuccgggcgagggugaaagcccucggaacccuagauugggucccucgggaucuuucuugagguuccuguggugcucaccugacccugcaagcaggaaaaagaaaaaagaggcgacggggaagaaguccuu

SEQ ID NO: 197 - RNA, 338 nt, Pelamoviroid
  MH974830.1 Peach latent mosaic viroid isolate T02-3, complete genome
uccaaaaguuucgccguaucucaacggcucgucagugggcuuagcccagacuuuugggagaaugaucaccucucagccccuccaccuugggguauccucuaucggacacuguaguuucgauggaaggguuaagcaccucgcaaugagguaaggugggacuuuuccuuuuggaaccaagcgguugguuccgaggggggugugauccagguaccgccguagaaacugaguuacgacgucuacccgggauucaaacccgguccccuccagaagugauucugggugaagagucugugcuaagcacacugacgagucucugaaaugagacgaaacucuuuuga

SEQ ID NO: 198 - RNA, 338 nt, Pelamoviroid
  MK212087.1 Peach latent mosaic viroid isolate y7-pgl6, complete genome
acccauaaguuucgccguaucucaacggcucgucagugggcuaagcccagacuuaugagagagugguuaccucucagccccuccaccuuggggugcccuauucggagcacugcaguuccgauagaaaggcuaagaacgucgcaaugacguaaggugggacucuuccuucuggaaccuagcgguugguuccgaggggggugugauccagguaccgccguagaaacugaguuacgacgucuacccgggauucaaacccgguccccuccagaagugauucuggaugaagagucugugcuuagcacacugacgagucucugaaaugagacgaaacucuucua

SEQ ID NO: 199 - RNA, 338 nt, Pelamoviroid
  MK212050.1 Peach latent mosaic viroid isolate y2-p5, complete genome
accauaaguuucgccguaucucaacggcucaucagugggcuuagcccagacuuaugggaguuuagucaccucucagccccuccaccuuggggugcccuauccgaagcacugcaguuucgguagaaaggcuaagcaucucgcaaugagauaaggugggacuuuuccuucuggaaccaagcgguugguuccgaggggggugugauccagguaccgccguagaaacuggauuacgacgucuacccgggauucaaacccgguccccuccagaagagauuuuggaugaaagagucugugcuaagcacacugaugagucuuugaaaugagacggaacuuuuuug

SEQ ID NO: 200 - RNA, 338 nt, Pelamoviroid
  KY355380.1 Peach latent mosaic viroid isolate PLMVd-MB-P2-19, complete genome
cccauaaguuucgucgcaucccagcgacucgucagugggcuaagcccagacuuaugagagaagugguuaccucucagccccuccaccuuggggugcccuauccgagcacugcugcuucgguagaaaggcuaagcacgucgcaaugacguaaggugggacuuuucccucggguuugagcggucgaacccagaggggagugugauccugguaccgccguagaaacuggauuacgacgucuacccgggauucaaacucggccccucucagaggugacucugagcgaaaggucugugcuuagcacacugacgaguuccugcaauggaacgaaaccuuucuaa

SEQ ID NO: 201 - RNA, 338 nt, Pelamoviroid
  KY355322.1 Peach latent mosaic viroid isolate PLMVd-JT-P2-1, complete genome
cccaaaaguuucgucgcaucccagcgacucaucagugggcuuagcccagacuuuugagagaagugguuaccucucagccccuccaccuuggggugcccuauccgagcacugcuguuucgguagaaaggcuaagaacgucgcaaugacguaaggugggacuuuucccuugaguucaagcgguuggacucagaggggagugugauccugguaccgccguagaaacuggauuacgacgucuacccgggauucaaacucggccccucucagaggugauucugagugaaaggucugugcuaagcacacugaugaguuccugcaauggaacgaaaccuuucaaa

SEQ ID NO: 202 - RNA, 338 nt, Pelamoviroid
  KY310586.1 Peach latent mosaic viroid isolate BC, complete genome
cucauaaguuucgccguaucucaacggcucaucagugggcuuagcccagacuuaugagagaguagucaccucucagccccuccaccuuggggugcccuauccgagacacugcuguuucgguagaaaggcuaagcacgucgcaaugacguaaggugggacuuuuccuucuggaaccaagcgguugguuccgaggggggugugauccagguaccgccguagaaacuggauuacgacgucuacccgggauucaaacccgguccccuccagaagugauucuggaugaagagucugugcaaagcacacugaugaguuccugaaauggaacgaaaccuuucuaa

SEQ ID NO: 203 - RNA, 338 nt, Pelamoviroid
  KF417551.1 Peach latent mosaic viroid isolate 96, complete genome
cacauaaguuucgccguaucucaacggcucaucagugggcuaugcccagacuuaugagagaguugugaccucucagccccuccaccuuggggugcccuauucgaggcacugcagucucgauagaaaggcuaagcacgucguaaugacguaaggugggacuuuuccuucgggaaccaagcgguugguuccgaggggggugugauccagguaccgccguagaaacuggauuacgacgucuacccgggauucaaacccgguccccuccagaagugauucuggaugaagagucugcgcauagcgcacugaugagucucugaaaugagacgaaacucuuuuca

SEQ ID NO: 204 - RNA, 338 nt, Pelamoviroid
  JX479733.1 Peach latent mosaic viroid clone 335_33_338 clone-lib P3, complete sequence
cuuuugagagaguaaugaccucucagccccuccaccuuggggugcccuauucggagcacugcaguucccgauagaaaggcuaagaaccucgcaaugagguaaggugggacuuuuccuucgggaaccaagcgguugguuccgaggggggugugauccagguaccgccguagaaacuggauuacgacgucuacccgggauucaaacccgguccccuccagaagugauucuggaagaugagucugugcuaagcacacugaugagucucugaaaugagacgaaacucauaucaucaaaaguuucgccgcauuucagcggcucaucagugggcuuagcccaga

SEQ ID NO: 205 - RNA, 338 nt, Pelamoviroid
  HM185115.1 Peach latent mosaic viroid isolate AY09AT5 clonH2, complete genome
cucaaaaguuucgccgcauuucagcggcucaucagugggcuaagcccagacuuuugagaaguagagauaccucucagccccuccaccuuggagugcccuauucggagcacugcaguucccgauagaaaggcuaagcaccucgcgaugagauaaggugggacucuuccuucuggaaccuagcgguagguuccgagggggugugauccagguaccgccguagaaacuggauuacgacgucuacccgggauucaaacccgguccccuccagaagugaauucuggaagaagagucugcgcuuagcgcacugaugagucucugagaugagacgaaacucuuca

SEQ ID NO: 206 - RNA, 338 nt, Pelamoviroid
  GQ499321.1 Peach latent mosaic viroid isolate 17, complete sequence
acucauaaguuucgucgcaucccagcgacucaucagugggcuuagcccagacuuaugggagauuaguuaccucucagccccuccaccuuggggugcccuauucggagcacugcaguucccgauagaaaggcuaagcacgucgcauugacguaaggugggacguuucccuaggguuugagcggucgaacccagggggagugugacccagguaccgccguagaaacuggguuacgacgccuacccgggauucaaacccggucccccucagaggugacucugagugaaaggucgcgcuaagcgcacugaugaguuccugaaauggaacgaaaccuuugaua

SEQ ID NO: 207 - RNA, 338 nt, Pelamoviroid
  DQ680760.1 Peach latent mosaic viroid variant 111.2, complete genome
accauaaguuucgccuuauuucaaaggcucaucagugggcuuagcccagacuuaugagagcuuaaacaccucucagcccuuccaccuuagggugcccuauucggagcacugcaguucccgauagaaaggcuaagcaccucgcaaugggguaaggugggacuuuuccuucuggaaccaagcgguugguuccgaggggggugugauccagguaccgccguagaaacuggauuacgacgucuacccgggauucaaacccgguccccuccagaagugauucuggaugaagagucugugcuaagcacacugacgagucucugaaaugagacgaaacucuucuc

SEQ ID NO: 208 - RNA, 338 nt, Pelamoviroid
  AF339740.1 Peach latent mosaic viroid wild pear isolate, complete genome
cucauaaguuccgccguaucucaacggcucaucagugggcuaagcccagacuuaugggagaguagucaccucucagccccuccaccuuggggugcccuauacggagcacuccaguucccgauagaaaggcuaagcaccucgcaaugagguaaggugggacuuuuccuucuggaaccuagcgguagguuccgaggggggugagauccagguaccgcuguagaaacuggauuacgacgucuaccagagauucaaacccgguccccccagaagugauucuggaugaagagucugugcuuagcacacugaugagucucugaaaugagacgaaacucuucuga

SEQ ID NO: 209 - RNA, 338 nt, Pelamoviroid
  EU708837.1 Peach latent mosaic viroid clone 215.1, complete genome
uccauaaguuucgccguaucucaacggcucaucagugggcuaagcccagacuuaugagagagugaugaccucucagccccuccaccuuggagugcgcuauucggagcacugcaguucccgauagaaaggcuaagcaccucgcaaugagguaaggugggacuuuuccuucuggaaccuagcgguagguuccgaggggggugugauccagguaccgccguagaaacuggauuacgacgucuacccgggauucaaacccgguccccuccagaaguguuccuggaugaagagucgcgcuuagcguacugaugagucucugaaaugagacgaaacucuucuca

SEQ ID NO: 210 - RNA, 337 nt, Pelamoviroid
  LC469622.1 Peach latent mosaic viroid RNA, complete genome
ucauaaguuucgucgcaucucagcgacucaucagugggcuuugcccagacuuaugagagaaaugaucaccucucagccccuccaccuuggggugcccuauccgagcacugcugcuucgguagaaaggcuaagcacgucgcaaugacguaagguggggcuuuucccuugaaguugagcggucgacuucagaggggagugugauccugguaccgccguagaaacuggauuacgacgucuacccgggauucaaacccggccccucucagaggugacucugagugaaaggucugugcuaagcacacugacgaguuccugcaauggaacgaaaccuuucuga

SEQ ID NO: 211 - RNA, 337 nt, Pelamoviroid
  KY355341.1 Peach latent mosaic viroid isolate PLMVd-JT-P2-23, complete genome
ucaaaaguuucgucgcaucccagcgacucaucagugggcuuugcccagacuuuugagaguagugaugaccucucagccccuccaccuuggggugcccuauccgagcacugcugcuucgguagaaaggcuaagcacgucgcaaugacguaaggugggacuuuucccuugaguucaagcgguuggacuccgaggggagugugauccugcuaccgccguagaaacuggauuacgacgucuacccgggauucaaacucggccccucucagaggugacucugagcgaaaggucugugcaaagcacgcugacgaguuccugaaauggaacgaaaccuuucuca

SEQ ID NO: 212 - RNA, 337 nt, Pelamoviroid
  KY355237.1 Peach latent mosaic viroid isolate PLMVd-CH-P2-20, complete genome
cucaaaaguuucgucgcaucccagcgacucaucagugggcuuagcccagacuuuugagagaaauagucaccucucagccccuccaccuuggggugcccuauccgaacacugcuguuucgguagaaaggcuaagcacgucgcaaugacguaaggugggacuuuucccuacaguucgagcggucgaacuagaggggagugugauccugguaccgccguagaaacuggauuacgacgucuacccgggauucaaacucggccccucucagaggugacucugagcgaaaggucugugcuaagcacacugaugaguuccugaaauggaacgaaaccuuuuuga

SEQ ID NO: 213 - RNA, 337 nt, Pelamoviroid
  KY355189.1 Peach latent mosaic viroid isolate PLMVd-BC-P2-10, complete genome
cccauaaguuucgccguaucucaacggcucaucagugggcuaagcccagacuuaugagagagugguuaccucucagccccuccaccuuggggugcccuauccgagacacugcuguuucgguagaaaggcuaagcacgucgcaaugacguaaggugggacuuuucccuacaguucgagcggucgaacuagaggggaguguaaccuugguaccgccguagaaacuggauuacgacgucuacccgggauucaaacccgguccccuccagaagugauucuggaugaagagucugugcuuagcacacugaugagucucugaaaugagacgaaacucuuuuaa

SEQ ID NO: 214 - RNA, 337 nt, Pelamoviroid
  KY310587.1 Peach latent mosaic viroid isolate CH, complete genome
aucaaaaguuucgucgcaucccagcgacucaucagugggcuuagcccagacuuuugagaguagugaugaccucucagccccuccaccuuggggugcccuauccgagcacugcuguuucgguagaaaggcuaagcacgucgcaaugacguaaggugggacuuuucccucuggaaccaagcgguugguuccgaggggggugugauccagguaccgccguagaaacuggauuacgacgucuacccgggauucaaacccgguccccuccagaagugauucuggaaaaagagucugugcuuagcacacugaugagucucugaaaugagacgaaacucuuuug

SEQ ID NO: 215 - RNA, 337 nt, Pelamoviroid
  KX430168.1 Peach latent mosaic viroid isolate v2.7, complete sequence
uccauaaguuucgucgcaucccagcgacucaucagugggcuuagcccagacuuaugagagaagugaugaccucucagccccuccaccuuggggugcccuauccgaagcacugcaguuucgguagaaaggcuaagaacgucgcaaugacguaaggugggacuuuucccuaggguuugagcggucgaacccagggggagugugacccagguaccgccguagaaacuggguuacgacgccuacccgggauucaaacccggucccccucagaggugacucugagugaaaggucgcgcuuagcgcacugaugaguuccugcaauggaacgaaaccuuucuca

SEQ ID NO: 216 - RNA, 337 nt, Pelamoviroid
  EU888599.1 Peach latent mosaic viroid isolate PchMx.Azt2, complete genome
ucauaaguuucgccguaucucaacggcucgucagugggcuaagcccagacuuaugagagaguagucaccucucagccccuccaccuuggggugcccuagucgagcacugcagucucgauggcaaggcuaaggacgucgcaaugacguaaggugggacuuuuccuucuggaaccaagcgguugguuccgaggggggugugauccagguaccgccguagaaacuggauuacgacgucuacccgggauucaaacccgguccccuccagaagugauucuggaugaagagucugugcuuagcacacugacgagucucugaaaugagacgaaacucuuuugac

SEQ ID NO: 217 - RNA, 337 nt, Pelamoviroid
  KF869589.1 Peach latent mosaic viroid clone 1128_8_337 clone-lib P3, complete sequence
ucaaaaguuucgccgcauuucagcggcucaucagugggcuuagcccagacuuuugagagaguaaugaccucucagccccuccaccuuggggugcccuauucggagcacugcaguucccgauagaaaggcuaagcacuucgcaaugaaguaaggugggacuuuucccuaggguuugagcggucgaacccagggggaguguaauccagguaccgccguagaaacuggauuacgacgucuacccgggauucaaacccgguccccuccagaagugauucuggaagaugagucugugcuaagcacacugaugagucucugaaaugagacgaaacucauauca

SEQ ID NO: 218 - RNA, 337 nt, Pelamoviroid
  JX480348.1 Peach latent mosaic viroid clone 950_9_337 clone-lib P3, complete sequence
cuuuugagagaguaaugaccucucagccccuccaccuuggggugcccuauucggagcacugcaguucccgauagaaaggcuaagcacuucgcaaugaaguaaggugggacuuuucccuaggguuugagcggucgaacccagggggagugugauccagguaccgccguagaaacuggauuacgacgccuacccgggauucaaacccgguccccuccagaagugauucuggaagaugagucugugcuaagcacacugaugagucucugaaaugagacgaaacucauaucaucaaaaguuucgccgcauuucagcggcucaucagugggcuuagcccaga

SEQ ID NO: 219 - RNA, 337 nt, Pelamoviroid
  JX479333.1 Peach latent mosaic viroid clone 1064_9_337 clone-lib P7, complete sequence
cacugcaguuucgauagaaaggcuaagcacuucgcaaugaaguaaggugggacuuuucccuaggguuugagcggucgaacccagggggagugugauccagguaccgccguagaaacuggauuacgacgccuacccgggauucaaacccggucccccucagaggugacucugagugaaaggucgugcuuagcacacugaugaguuccugaaauggaacgaaaccuuuuuaacccuauaaguuucgucgcaucccagcgacucaucagugggcuaagcccagacuuaugagagagugguuaccucucagccccuccaccuuggggugcccuauucggag

SEQ ID NO: 220 - RNA, 337 nt, Pelamoviroid
  AJ550900.1 Peach latent mosaic viroid complete genome, isolate PC-C34
aacauaaguuucgccuugucgcaaaggcucaucagugggcuuagcccagacuuaugugagaguuuaacaccucucagcccuuccaccuuagggugcccuauucggacacugcaguuucgauagaaaggcuaagcaccucgcaaugagguaaggugggacuuuuccuucgggaaccaagcgguugguuccgaggggggugugauccagguaccgccguagaaacuggauuacgacgucuacccgggauucaaacccgguccccuccagaagugauucuggaagaagagucugcgcuaagcgcacugaugagucuuugagauaagacgaaacucuucua

SEQ ID NO: 221 - RNA, 337 nt, Pelamoviroid
  AF170523.1 Peach latent mosaic viroid variant #4 from Prunus persica Tylor cultivar, complete sequence
aucaaaaguuucgccguaucucaacggcucaucaguggcuaagcccagacuuuugagagaagugaugaccucucagcccuuccaccuuggggugcccuauucggagcacugcaguucccgauagaaaggcuaagcacgucgcaaugacguaaggugggacuuuuccuucuggaaccaagcgguugguuccgaggggggugugacucagguaccgccguagaaacugaguuacgacgucuacccggaauucaaucccguccccuccagaagugauucuggaagauaagucugugcuuagcacacugaugagucucugaaaugagacgaaacuuaucuc

SEQ ID NO: 222 - RNA, 336 nt, Pelamoviroid
  MH974831.1 Peach latent mosaic viroid isolate T02-6, complete genome
cccaaaaguuucgucgcaucgcagcgacucaucagugggcuuagcccggacuuuugagagaaauggucaccucucagccccuccaccuuggggugcccuauccgaacacugcuguuucgguagaaaggcuaagcacgucgcaaugacguaaggugggacuuuucccuugaguuugagcgguugguuccgaggggggugugauccagguaccgccguagaaacuggauuacgacgucuuaccgagauucaaacucggccccucucagaggugacucugagcgaaaggucgcgcuaagcgcacugaugaguuccugaaauggaacgaaaccuuucaga

SEQ ID NO: 223 - RNA, 336 nt, Pelamoviroid
  KY355241.1 Peach latent mosaic viroid isolate PLMVd-CH-P2-24, complete genome
cucaaaaguuucgucguaucucaacggcucaucagugggcuaagcccagacuuaugagagauuagucaccucucagccccuccaccuuggggugcccuauucggacacugcaguuccgauagaaaggcuaagaacgucgcaaugacguaaggugggacuuuuccuucuggaaccaagcgguugguuccgaggggggugugauccagguaccgccguagaaacuggauuacgacgucuacccgggauucaaacucggccccucucagaggugacucugagcgaaaggucgugcuaagcacacugaugaguuccugaaauggaacgaaaccuuucuga

SEQ ID NO: 224 - RNA, 336 nt, Pelamoviroid
  JX479340.2 Peach latent mosaic viroid clone 1071_9_336 clone-lib P7, complete sequence
cccauacuuaugagagagugguuaccucucagccccuccaccuuggggugcccuauucggagcacugcaguuccgauagaaaggcuaagcacuucgcaaugaaguaaggugggacuuuucccuaggguuugagcggucgaacccagggggagugugauccagguaccgccguagaaacuggauuacgacgccuacccgggauucaaacccggucccccucagaggugacucugagugaaaggucgugcuuagcacacugaugaguuccugaaauggaacgaaaccuuucuaacccauaaguuucgucgcaucccagcgacucaucagugggcuaag

SEQ ID NO: 225 - RNA, 336 nt, Pelamoviroid
  AF339739.1 Peach latent mosaic viroid peach isolate, complete genome
aucauaaguuucgccguaucucaacgccucaucagugggcuaagcccagacuuaugagagaguaaagaccucucagccccuccaccuuggggugcccuauucggagcacugcaguucccgcuagaaaggcuaagcaccucgcaaugagguaaggugggacuguuccuucggaaccuagcgguuggcuucgaggggggugugacucagguaccgccguagaaacugaguuacgacgucuacccgggauucaaacccguccccuccagaagugauucuggaugaagagucugugcuuagcacacugaugagucucugaaaugagacgaaacucuucuc

SEQ ID NO: 226 - RNA, 335 nt, Apscaviroid
  FJ974092.1 Apple scar skin viroid clone Leuki 30.1, complete genome
gguaaacaccgugcgguuccugugguucgccccgccaacgcagauagauaaagaaaacgaggagaagaaggaacucaccugucgucgucgacgaaggccggugagaaaggagcugccagcacuaaggcggacggcguagucgcgauagcagcguuccgcugucgucuccacugcaagaacguacgguguugaggcccuguccgccgcugcgcugccaccuacucucgcgccgcuagucgagcggacuccggguggagcccccuguucucucacgcucuuuuucuuugacgcagcggcggguggguucccaggguaaaacacaauagguguuuccc

SEQ ID NO: 227 - RNA, 335 nt, Elaviroid
  KT901908.1 Eggplant latent viroid clone ELVd2-74, complete genome
gggugggguuggccaccccuugaugagaccaaaagguggaaaugggguuucgccaugggucggaacuuuaaauucggaggauucguccuuuaaacguuccuccaagaguuccuuccccaaacccuuacuuucguaaguuugguucggcgaauggaccguuucguccuuuuggacucaucagggaaaguacacacuuuccgacgguggguucgucgacaccucucccccccccagguacuaucccuuuccaaggauguguucccuagggggguggguguaccucuuuuggauugcuccggccuuccaggagagauagaggacgaccucuccccaua

SEQ ID NO: 228 - RNA, 335 nt, Elaviroid
  KT901890.1 Eggplant latent viroid clone ELVd2-56, complete genome
ggguggugugugccaccccugaugagaccgaaaggucgaaaugggguucugccauggguagggucuuuaaauucggaggauucguccuuuaaacguuccuccaagagacccuaccccaaacccuuacuuauguaagaaagguucggcgaauguaccguuucguccuuucggacucaucagggaaaguacacacuuuccgacgguggguucgucgacaccucucccccucccagguacuaucccuuuucaaggauguguucccuaggagggugggcguaccucuuucuggauugcuccggccuuccaggagagauagaggacgaccucuccccaua

SEQ ID NO: 229 - RNA, 335 nt, Elaviroid
  KT901868.1 Eggplant latent viroid clone ELVd2-34, complete genome
ggguggugugugccaccccugaugagaccaaaaggucgaaaugggguuucgccaugggucuggucuuuaaauucggaggaaucguccuuuaaacguuccuccaagagaccauuccccaaacccuuacuuauguaagaaugguucggcgaauguaccguuucgcucuuuugagcucaucagggacaguacacacuguccgacgguagagucgucgacaccucucccccccccagguacuauccucuuucaaggauguguucccuagggggguggguguaccucuuuucggauuacuccggucuuccaggagagauagaggacgaccucuccccaua

SEQ ID NO: 230 - RNA, 335 nt, Elaviroid
  AJ536620.1 Eggplant latent viroid complete genome, isolate 9
ggguggugugugccaccccugaugaguccgaaaggacgaaauggggucucuccaugggucggaaccuuaaauucggaggauucguccuuuaaacguuccuccaaggguuccuuccccgaucccuuacuuucguaaguauggaacggagaguguaccguuucgcccuuuugggcucaucagggaaagcgcacacuuuccgacggugggguuguugacaccucucccccucccagguacuaucccuuuucaaggauguguucccuaggaggguggguguaccucuuucuggauuucuccggucuuccaggagagguagaggacgaccucuccccaua

SEQ ID NO: 231 - RNA, 335 nt, Elaviroid
  AJ536617.1 Eggplant latent viroid complete genome, isolate 6
gggugguguguaccaccccugaugaguccgaaaggacgaaaugggguucugccaugggguagagcuuuaaauucggaggauucgaccuuaaaucguuccuccaagagcucuucccccaaucccuuacuuguguaaguacggaucggcggauguaccguuucgcccuuucgggcucaucagggaaagcgcacacuuuccgacggugaguucgucgacaccucugccccucccagguacuauccucuuucaaggauguguucccuaggagggugaguguaccucuuuucggauuucuccggucuuccgagagagacagaggacggccucuccccaua

SEQ ID NO: 232 - RNA, 335 nt, Pelamoviroid
  AJ241843.1 peach latent mosaic viroid, varient esc10-6b
aucaaaaguuucgccgcauuucaguggcucaucagugggcuaagcccagacuuaugagagagugguuaccucucagccccuccaccuuaggaugcccuauucggacaugcaguuccgauggaaaggcuaagcaccucgcaaugagguaaggugggacuuuuccuucgggaaccaagcgguugguuccgaggggggugugauccagguaccgccguagaaacuggauuacgacgucuacccgggauucaaacccgguccccuccagaagugauucuggaagaugagucugugcuaagcacacugaugagucucugaaaugagacgaaacucauauc

SEQ ID NO: 233 - RNA, 334 nt, Apscaviroid
  MN598209.1 Apple scar skin viroid isolate ASSVd PQ-n, complete sequence
ccggugagaaaggagcugccagcacuaagccggacggcgcccucgcaccagauccucugugggcuugcccucaagaucgcaucgguguugaggcccuguccgccgcugcgcugccaccuacucuucgcgccgcuagucgagcggacuccaggugggagcuucccuauucucucacgcucuuuucuuuuugcgcagcggcggguggguucccaggguaaaacacaauugguguuucccgguaacaccgugcgguuccugugguucgccccgccaaagcauaaauuuuaaagaaaacgaggagaagaaggaacucaccugucgucgucgacgaagg

SEQ ID NO: 234 - RNA, 334 nt, Apscaviroid
  MK102984.1 Apple scar skin viroid isolate QDHT-4, complete genome
gguaaacaccgugcgguuccugugguucgccccgccaaagcauaaauuuuaaagaaaacgaggagaagaaggaacucaccugucgucgucgacgaaggccggugagaaaggagcugccagcacuaagccggacggcgcccucgcaccaguuccgcuguggguucgccuacaagaacguacgguguugaggcccuguccgccgcugcgcugccaccuacucuucgcgccgcuagucgagcggacuccaggugggagcuucccaauucucucacgcucuuuucuuuuugcgcagcggcggguggguucccaggguaaaacacaauugguguuuccc

SEQ ID NO: 235 - RNA, 334 nt, Apscaviroid
  HQ326087.1 Apple scar skin viroid isolate AY106, complete genome
gguaaacaccgugcgguuccugugguucgccucgccaaagcauagauuuuaaagaagacgaggagaagaaggaacucaccugucgucgucaacuaaggccggugagaaaggagcugccaccacuaagccggacggcacccucgcgacgaguuccgcugcggugucgccugcaauaacgcaugguguugaggcccuguccgccgcugcgcugccaccuacucuucgcgccgcuagucgagcggacuccagguggagauucccuauucucucaagcucuuuucuuuuugcgcagcggcgaguggguucccaggguaaaacacaauugguguuuccc

SEQ ID NO: 236 - RNA, 334 nt, Apscaviroid
  EU144226.1 Citrus bent leaf viroid isolate Anliucheng-1, complete genome
cggagacuucuugugguuccuguggugacaccccucagcccuaccugcgaaagaaaaaagaguuagaaggcggcagaggagcugacuggucgucgucgacgaaggcucgucagcugcggagguuggggucgacuggccuccgguggcgaagcugaguugagcuucgcugcuaucucuucuaagcuguaaccgagaccgguccccuucacccgagcgcugcuugccgcuagucgagcggacuuccaagucugaccucccgagccgcuuuucuuuucuccugaauuuccuguagcagcggggagagggugaagccccugaaccccugagggcuccu

SEQ ID NO: 237 - RNA, 334 nt, Elaviroid
  KT901928.1 Eggplant latent viroid clone ELVd2-94, complete genome
ggguggugugugccaccccugaugagaccgaaaggucgaaaugggguuucuccauggguaggaacuuuaaauucggaggauucguccuuuaaacguuccuccaagaguuccuuccccaaacccuuacuuuuguaaguuuggaucggcgaauguaccguuucguccuuucggacucaucagggacaguacacacuuuccgacggcggauucgucgacaccucucccccccccagguacuauccuuuuucaaggauguguucccuagggggguggguguaccucuuuuggauuucuccggccuuccaggagagauagaggacgaccucuccccaua

SEQ ID NO: 238 - RNA, 334 nt, Elaviroid
  KT901909.1 Eggplant latent viroid clone ELVd2-75, complete genome
ggguggugugugccaccccugaugagaccgaaaggucgaaaugggguuucgcuauggguuggaaccuuaaauucgggggauucguccuucaaacguucccccaaggguuccuuccccaaucccuuacuuuuguaaguacggaucggcgaauguaccguuucguccuuucggacucaucagggaaaguacacacuuuccgacgguggguucgucgacaccucucccccucccagguacuaucccuuuucaaggauguguucccuaggaggguggguguaccucuuuuggauugcuccggccuuccaggagagauagaggacgaccucuccccaua

SEQ ID NO: 239 - RNA, 334 nt, Elaviroid
  KT901902.1 Eggplant latent viroid clone ELVd2-68, complete genome
ggguggugugugccaccccugaugagaccgaaaggucgaaaugggguuuccccgugggucggauuuuuuuauucugaggauucguccuuuaaacguuccuccaagaguuccuuccccaaacccuuacuuaaguaauuacgguucggcgaauguaccguuuccuccuuuccgacucaucagggaaaguacacacuuuccgacgguggguucgucgacaccucucccccucccagguacuaucccuuuucaaggauguguuccaaaggaggguggguguaccucuuuuggauugcuccggccuuccaggagagauagaggacgaccucuccccaua

SEQ ID NO: 240 - RNA, 334 nt, Elaviroid
  KT901858.1 Eggplant latent viroid clone ELVd2-24, complete genome
ggguggugugugccaccccugaugagaccgaaaggucgaaaugggguucugccaugggucgggacuuuaaauucggaggaaucguccuuuaaucguuccuccaagggucccuuccccaaacccuuacucugguaagaaagguucggcggauguaccguuucguccuuucggacucaucagggaaagcacacacuuuccgacgguggggucguugacaccucucccccccccagguacuauccuuuuccaaggauguguucccuagggggguggguguaccucuuuuggauugcuccggucuuccaggagagauagaggacgaccucuccccaua

SEQ ID NO: 241 - RNA, 334 nt, Elaviroid
  AJ536615.1 Eggplant latent viroid complete genome, isolate 4
gggugguguguaccaucccugaugaguccaaaaggacgaaaugggguuucuccaugggucgggacuuuaaauucggaggauucguccuuaaaacguuccuccaagagucccuuccccgaucccuuacuuauguaagaaaggaacggagaauguaccguuucgcucuuucgggcucaucagggaaagcgcacacuuuccgacggugaguucgucgacaccucucccccccccggguacuuucccuuuucaaggaaguguuccccagggggguggguguaccucuuuuggauugcuccggucuuccaggagagauagaggacgaccucuccccaua

SEQ ID NO: 242 - RNA, 334 nt, Pospiviroid
  MF770198.1 Columnea latent viroid isolate TS03, partial genome
  misc_feature (4)..(4): n is a, c, g, or u
  misc_feature (27)..(27): n is a, c, g, or u
gugnuacugacugcagccaugcaaganaaaagaacgggagaagaagcguaagagcggucucaggagccccggggcaacucaaaccgagcggggucuugaccaguggcgagcgcccuguucaaacaggaguaaucccagcugaaacaggguuuucacccuuccuuucuucugguuuccuuccucugcuucagcggccucgcccggagucuugaccagcgcagguucugacgcgaccgguggcaucaccgaguuucgcucaaaccucaaucuccuuuuucuucauucuacuugggucuccgggcaaggggguuuaccccuugaaaccgcauuugga

SEQ ID NO: 243 - RNA, 333 nt, Apscaviroid
  MN598207.1 Apple scar skin viroid isolate ASSVd PQ-hg, complete sequence
ccggugagaaaggagcugccagcacuaagccggacggcgcccucgcaccaguuccgcuguggguucgccuacaagaacguacgguguugaggcccuguccgccgcugcgcugccaccuacucuucgcgccgcuagucgagcggacuccagguggagcuucccugaucucucacgcucuuuuucuuuugcgcagcggcggguggguucccaggguaaaacacaauagguguuucccgguaaacaccgugcgguuccugugguucgccccgccaaagcaauaaugauaaagaaaacgaggagaaaaaggaacucaccugucgucgucgacgaagg

SEQ ID NO: 244 - RNA, 333 nt, Apscaviroid
  KC769198.1 Citrus viroid VI isolate SE-1, complete sequence
ggaggaaacuccgugugguuccugugggucaccccccggcacccucuaaugaaaagaaaggcaaggagaacucaccugucgucgucgacgaaggcaugugagcuucaaucuccgugaagagcgucagcggacggccucugagacgagcgauggacaguagagcucgucucuaccagucgcgugcugacucucacgcccacccgcacggcugcuccgagagaggagagcucucugccgcuagucgagcggacuaacagagugacucuccugcccuauuuucuacggagagccggcgguggaguccccaggguaaaacacgauugguguuucccc

SEQ ID NO: 245 - RNA, 333 nt, Elaviroid
  KT901918.1 Eggplant latent viroid clone ELVd2-84, complete genome
ggguggugugugccaucccugaugagaccaaaaggucgaaaugggguuucgccaugggucuggucuuuaaauucggaggauucguccuuuaaacguuccuccaaggguccucuccccaaucccuuacuuaguaaguuuggaucggcgaguguaccguuucguccuuucggacucaucagggacaguacacacuguccgacggcagagucgucgacaccucucccccccccagguacuaucccuuuucaaggauguguucccuagggggguggguguaccucuuuuggauugcuccggucuuccaggagagauagaggacgaccucuccccaua

SEQ ID NO: 246 - RNA, 333 nt, Elaviroid
  KT901916.1 Eggplant latent viroid clone ELVd2-82, complete genome
ggguggugugugccaccccugaugagaccgaaaggucgaaaugggguucugccaugggaagggaccuuaaauucggaggauucgucccuuaaacguuccuccaaggguccuuuccccaaucccuuacuuaguaaguacggaucggcggauguaccguuucguccuuucggacucaucagggacaguacacacuguccgacgguagagucgucgacaccucucccccccccagguacuauccuuuuccaaggauguguucccuagggggguggguguaccucuuuuggauuucuccggucuuccaggagagauagaggacgaccucuccccaua

SEQ ID NO: 247 - RNA, 333 nt, Elaviroid
  KT901903.1 Eggplant latent viroid clone ELVd2-69, complete genome
ggguggugugugccaccccugaugaaaccaaaaggucgaaauggggguucgccaugggucgggacuucaaauucggaggauucguccuuuaaacguuccuccaagagucccuuccccaaacccuuacuuuguaagaaugguucggcgaauguaccuuuucuuccuuucggacucaucagggaaaguagacacuuuccgagggugaguuggucgaccccucucccccucccagguacuaucccuuugcaaggauguguucccgaagaggggggguguaccuuguuuggauugcuccggccuuccacgagaaauagaggacgaccucuccccaua

SEQ ID NO: 248 - RNA, 332 nt, Apscaviroid
  KP765435.1 Apple scar skin viroid isolate Yeongwol, complete genome
gguaaacaccgugcgguuccugugguucgccccuuaaaggcagauagauaaagaaaacgaggagaagaaggaacucccuuguaagucgucgacgaaggccggugagaaaggagcugccagcacuaagccggacggcgcccucgcaccaguuccgcuguggguucgccuacaagaacguacgguguugaggcccuguccgccgcugcgcugccaccuacucuucgcgccgcuagucgagcggacuccggguguagcccccuguucucucacgcucuuuuucuuugacgcagcggcggguggguucccaggguaaaacagaauagguguuucgc

SEQ ID NO: 249 - RNA, 332 nt, Apscaviroid
  EU031486.1 Apple scar skin viroid variant ASSVd_PE10, complete genome
gguaaacaccgugcgguuccuauagggcgacccgccaacgcagauagauaaagaaaacgagccgaagaaggaacucaccugucgucgucgacgaaggccggugagaaaggagcugccagcacuaagccggacggcgcccucgcaccgaguuccgcuguggguucgccugcaagaacgcaugguguugaggcccuguccgccgcugcgcugccaccuacucucgcgccgcuagucgagcggacuccggguggagcccccuguucucucacgcucuuuuucuuugacgacgcggcggguggguuccgcaggguaaaacacaauagguguuuccc

SEQ ID NO: 250 - RNA, 331 nt, Apscaviroid
  MN598214.1 Apple scar skin viroid isolate ASSVd LY-qb, complete sequence
ccggugagaaaggagcugccagcacuaagccggacggcgcccucgcaccgaguuccgcugcggguucgccugcaagaacgcaugguguugaggcccuguccgccgcugcgcugccaccuacucuucgcgccgcuagucgagcggacuccggguguagcccccuguucucucacgcucuuuuucuuugacgcagcggcggguggguucccaggguaaaacacaauugguguuucccgguaacaccgugcgguuccugugguucgccccgccaacgcagauagauaaagaaaacgaggagaagaaggaacucaccugucgucgucgacgaagg

SEQ ID NO: 251 - RNA, 331 nt, Apscaviroid
  MH476217.1 Australian grapevine viroid, complete genome
uaagacucaccuggcgacucgucgucgacgaaggguccucagcagagcaccggcaggaggcgcuaugcaggaacgcuagggguccuccagcggaggacugaagaaacuccgguuucuucuuucacucuguagcuggaaucccuguugcgcuugcuggcgaaaccugcagggaagcuagcugggucccgcuagucgagcggacucgucccagcggucccaaccaguuuucuuuauccuauuuuucccugcgggcgcccggucgugguuacccuggaacccccuuguuuggaggcccugggcaccaacuagugguuccugugguacucaccga

SEQ ID NO: 252 - RNA, 331 nt, Apscaviroid
  LC431516.1 Apple scar skin viroid GH1 RNA, complete genome
gguaaacaaccugcgguuccugugguuccccccgcccacgcagauagauaaagaaaacuaggaaaauauugaauucaccuguccucgucgacgaaggccggugagaaaggagcugccagcacuaagccggacggcgcccucgcaccaguuccgcuguggguucgccuacaagaacguacgguguugaggcccuguccgccgcugcgcugccaccuacucuucgcgccgcuagucgaucggacuccggguguagcccccuguucucucacgcucuuuuucuuugacgcaccggcggguggguucccaggguaaaucauaauagguguuuccc

SEQ ID NO: 253 - RNA, 331 nt, Apscaviroid
  LN823956.1 Apple scar skin viroid, complete sequence, isolate DHWH1
gucgacgaaggccggugagaaaggagcugccagcacuaagccggacggcgcccucgcaccaguuccgcuguggguucgccuacaagaacguacgguguugaggcccuguccgcugcugcgcugccaccuacucuucgcgccgcuagucgagcagacuccggguggagcccccugaucucccucgcucuuuuucuuugacgcagcggcggguggguucccaggguaaaacacaauagguguuucccgguaaacaccgugcgguuccugugguucgccccgccaacgcagauagauaaagaaaacgaggagaagaaggaacucaccugucguc

SEQ ID NO: 254 - RNA, 331 nt, Elaviroid
  KT901906.1 Eggplant latent viroid clone ELVd2-72, complete genome
gggugaugugugccgccccugaggagaccgaaagguccaaaugggguuucuccaugggucgggacuuuaaauucggaggauucguccuucaaacguuccuccaagaguccuuuccccaaacccuuacguuguaaguaggguucggcgaaaguacgguuucguccuuucggacucaucagggaaaguacacgcuuuccgacgguggguucgucggugcucucccccucccagguacuaucccuuuucaaggauguguuccgcacgaggguggguguacaucuuuuggauggcuccugcauucugaagagauagaggacgaccucuccccaua

SEQ ID NO: 255 - RNA, 330 nt, Apscaviroid
  LN823957.1 Apple scar skin viroid, complete sequence, isolate DHWH2
gucgccgaagaccggugagaaaggagcugccagcacuaagccggacggcgcccucgcaccaguuccgcuguggguucgccuacgagaacguccgguguugaggcccaguccgcugauccgcugccaccuacucuucgcgccgcuagucgagcggacuccgaguggacccccuuguucucccucgcucuuuuucuuugacgcagcggcggguggguucccaggguaaaacacaauagguguuuccagguaaacaccgugcgguccugugguucgccccgccaacgcagauagauaaagaaaacgagucguuuaaggaacucaccugucguc

SEQ ID NO: 256 - RNA, 330 nt, Apscaviroid
  EU031496.1 Apple scar skin viroid variant ASSVd_AP10, complete genome
gguaaacaccgugcgguuccucagguucgccacgccaacgcagcuacauauagaaaacgaauugaagaaugaauucccaugucgucgucgacgaaggccggugagaaaggagcugccagcacuaagccggacggcgcccucgcaccaguuccgcuguggguucgccuacaagaacguacgguguugaggcccuguccgccgcugcgcugccaccuacucucgccgcgcuagucgagcggacuccggguggagcccccuguucucucacgcucuuuuucuuugacgcagcggcggguggguucccaggugaaaacacaauagguguuuccc

SEQ ID NO: 257 - RNA, 330 nt, Apscaviroid
  EU031466.1 Apple scar skin viroid variant ASSVd_AM12, complete genome
gguaaacaccgugcgguuccugugguucgcgccgccaacgcagauagauaagaccaacgaggagaagaaggaacucaccugucgucgucgacgaaggccggugagaaaggagcugccagcacuaagccggacggcgcccucgcaccaguuccgcuguggugucgccuacaagaacguacgguguugaggcccuguccgccgcugcgcugccaccuacucugcccgcgcuagucgagcggacuccggguggagcccccuguucucucacgcucuuuuucuuugacgcagcggcggguggguucccaggguaaaacacaauagguguuuccc

SEQ ID NO: 258 - RNA, 330 nt, Apscaviroid
  AB054640.1 Citrus viroid-I-LSS variant WS2 genomic RNA, complete sequence
cggagacuucuugugguuccuguggugacaccccucagcccuaccugcgggcgaaagaaaaaagacauuagaggcuccagaggagacugagcggucgucgucgacgaaggcuccucagucgcagagcgccgcuggaucgacuggccuccgguggaaagaagaagcuuugucuucaauuucuguaaccggaccggucuccuucggccgccgagcgcugguagccgcuagucgagcggacuuccgacucuucccucccgaggagcuuucuucuugucugaauuccguagcagcggggagagggugaagcccugugaaccccugagggcuccu

SEQ ID NO: 259 - RNA, 329 nt, Apscaviroid
  MH013320.1 Apple scar skin viroid isolate JZ1-4, complete genome
gacgaaggccggugagaaaggagcugucagcacuaagccggacggcgcccucgcaccaguuccgcuguggguucgccuacaagaacguacgguguugaggcccuguccgccgcugcacugccaccuacucuucgcgccgcuagucgagcggacuaagguggacuucccuauucucucacgcucuuuucuuuuugugcagcggcggguggguucccaggguaaaacacaauugguguuucccgguaacaccgugcgguuccugugguucgccccgccaaagcauauaauuaaagaaaacgaggagaagaaggaacucaccugucgucguc

SEQ ID NO: 260 - RNA, 329 nt, Apscaviroid
  KY114496.1 Australian grapevine viroid isolate TR188-Antep Karasi, partial genome
ggucccaaccaguuuucuuuauccuauuuuucccugcgggcgcccggucgugguuacccuggagcucccuguuuggaggcccugggcaccaacuagagguuccugugguacucaccgaaggccgcgaagcuaggaaagaaaaagauagaaaagcuggguaagacucaccuggcgacucgucgucgacgaaggguccucagcagagcaccggcaggaggcgcuaugcaggaacgcuagggguccuccagcggaggacugaagaaacuccgguuucuucuuucacucuguagcuggaaucccuguugcgcuugcuggcgaacccugcaggg

SEQ ID NO: 261 - RNA, 329 nt, Apscaviroid
  KY114494.1 Australian grapevine viroid isolate TR179-Antep Karasi, partial genome
gggccugcagggauuucuuuauccuauuuuucccugcgggcgcccggucgugguuacccuggagcucccuguuuggaggcccugggcaccaacuagagguuccugugguacucaccgaaggccgcgaagcuaggaaaaaaaaaaauaaaaaagcuggguaagacucccugggcaacucgucuucaacaaaggguccucaccaaacccccggcgggaggcguuuugcaggaacgcuagggguccuccagcgaagaauugaaaaaacuccgguuucuucuuucauucuguaguuggaauuccugguggccuugguggcaaccccugcaggg

SEQ ID NO: 262 - RNA, 329 nt, Apscaviroid
  KY114492.1 Australian grapevine viroid isolate TR189-Ceviz Uzumu, partial genome
gaccugccagaaaggaccucuugcggugcaaacccgggggggacacguggugcuugcuguggaaccccguggggagaggcccugggcaccaacuagagguuccugugguacucaccgaaggccgcgaagcuaggaaagaaaaagauagaaaagcuggguaagacucaccuggcgacucgucgucgacgaaggguccucagcagagcaccggcaggaggcgcuaugcaggaacgcuagggguccuccagcggaggacugaagaaacuccgguuucuucuuucacucuguagcuggaaucccuguugcgcuugcuggcgaaaccugcaggg

SEQ ID NO: 263 - RNA, 329 nt, Apscaviroid
  KP772685.1 Apple scar skin viroid isolate S-q6-10, complete genome
ugagacuggagcugccaccacuggugcggacgguccccuccccguuuuuccgcugugucugcgccuacggaacgaacgguguugaggcccuguccgccgcugcgcugccaccuacucucgcgccgcuaggaaagcggacuccggguggagcccccuguuaucucacgcucuuuuucuuugacgcccgggcgggugucuucccaggguaaaacacaauagguguuucccgguaaacaccgugcggcuccuguggaucgccccgccaacgaacauacauaaaaaaugcgaggagaacaacgaccucaccuguggucgucgaucauugccgg

SEQ ID NO: 264 - RNA, 329 nt, Apscaviroid
  KF726092.1 Citrus viroid-I-LSS isolate SL-4, complete genome
cggagacuucuugugguuccuguggugacaccccucagcccuaccugcgaaagaaaaaagucauuagaaggcgccagaggagacugagcggucgucgucgacgaaggcuccucagucgcagagcgccgcuggaucgacuggccuccgguggaaaacgaagauucgucuucaauuucuguaaccggaccggucuccuucggccgccgagcgcugguugccgcuagucgagcggacuuccgucucuacccuccccgaggcgcuuuuucugauugaccgacuuccguagcagcggggagagggugaagcccugugaaccccugagggcuccu

SEQ ID NO: 265 - RNA, 329 nt, Apscaviroid
  HG764206.1 Apple scar skin viroid, complete sequence, isolate Q1
gguaaacaccgugcgguuccugugguucgccccgccaacgcagauagauaaagaaaacgaggagaagaaggaacucaccugucgucgucgacgaaggccggugagaaaggagcugccagcacuaagccggacggcgcccucgcaccaguuccgcuguggguucaccugcaagaacguacgguguugaggcccuguccgcugccgcugccaccuacucuucgcgccgcuagucgagcggacuccggguggagcccccuguucucccacgcucuuuuucuuugacuaggcggcgggagaguucccagaguaaaacacaauagguguuuccc

SEQ ID NO: 266 - RNA, 329 nt, Apscaviroid
  GQ249350.1 Apple scar skin viroid isolate Vassiliki-680, complete genome
gguaaacaccgugcgguuccugugguucgccccgccaacgcagauagauaaagaaaacgaggagaagaaggaacucaccugucgucgucgacgaaggcggugacaaaggagcugccagcaguaagccugacucggucgcaccaguuccgcugugcguucgccuacgaaggcaacguacgguguugaggcccuguccgccgcugcgcugccaccuacucucgcgccgcuagucgagcggacuccggguggagcccccuguucucuuaaacuaauguacuuugacgcagcggcggguggguucccaggguaaaacacaauagguguuuccc

SEQ ID NO: 267 - RNA, 329 nt, Apscaviroid
  M36646.1 Apple scar skin viroid, complete sequence
gguaaacaccgugcgguuccugugguucgccccgccaacgcagaagauaaagaaaacgaggagaagaaggaacucaccugucgucgucgacgaaggccggugacaaaggagcugccagcacuaagccggacgcgcccucgcaccaguuccgcugugggccccccuacaagaacguacggugcugaggcccuguccgccgcugcgcugccaccuacucuucgcgcccucagucgaccggacuccggguguagcccccuguucucucacccucuuuuucuuugacgcagcggcggguggguucccaggguaaaacacaauaccuguuuccc

SEQ ID NO: 268 - RNA, 329 nt, Pospiviroid
  MF770202.1 Columnea latent viroid isolate TS08, partial genome
guccugaccugcagccaugcaagaaaagaacgggaggaagagcgcaagaacggucucaggagccccggggcaacucaaaacgagcggggaucgcggaccgagggcagaagccugcuucaaacaggaguaauccccgcugaaacaggguuuucacccuuccuuucuucggguuuccuuccucugcuugcggccucccccggaaucuucgaauccagcgcagguucagacgcgaccgggggcccgccaagucgcucacugccucacccuccuuuuucucuaucuagcuuggucuccgggcgaggguguuuagcccuuggaaccgcaguugg

SEQ ID NO: 269 - RNA, 328 nt, Apscaviroid
  KP772687.1 Apple scar skin viroid isolate S-T37-11, complete genome
ugagaaaggaguucagggcgcuuccauugaccauucccucgcaccaguuccggggaggggcgaucgcaggaaccucuucguugaggccccauccggcgaaagggggaugccuacucuggcgauuaaaguggagcggaccaggguuggagcagucuguucuuucacgcucuuuuuccagggagcaguggcggguggguucccaggguaaaacacaauagguguuucccgguaaacaccgucauguuggccgcguuaauucgaccaacgcagauugauaaagaaaacgcagagaagacggaagggagagcucguaguggacgaaggccgg

SEQ ID NO: 270 - RNA, 327 nt, Apscaviroid
  MH200820.1 Citrus bent leaf viroid isolate CBLVd-GH-Cl13, complete genome
ggcucgucagcugcggagguuggggucgacuggccuccgguggcgaagcugaguugagcuucgcucuucucuucuuagcuguaaccggaccgguccccuucacccggcgcugcuugccgcuagucgcgcggacuuccaagucucccucccgagccgcuuuucuuuucuccugauuuccguagcagcggggagagggugaagccccugaaccccugacggcuccucggagacuucuugugguuccuguggugucaccccucagcccuaccugcgaaagaaaaaagaguuagaaggcggcagaggagcugacuggucgucgucgacgaa

SEQ ID NO: 271 - RNA, 327 nt, Apscaviroid
  JX861259.1 Apple scar skin viroid clone ASSVd-Z7, complete genome
uaaacaccgugcggucccugugguucgccccgccaaagcauauaauuaaagaaaacgaggagaagaaggaacucaccugucgucgucgacgaaggccggugagaaaggagcugccagcacuaagccggacggcgcccuugcaccaguuccucugugggcuugcccucaagaacgcuacgguguugaggcccuguccgccgcugcgcugcacgccucucuucgcgccgcuagucgagcggacuacgggcgguuucccuauucucucacgcucuuuucuuuuugugcagcggcggguggguucccaggguaaaacacaauagguguuuc

SEQ ID NO: 272 - RNA, 323 nt, Pelamoviroid
  KF870227.1 Peach latent mosaic viroid clone 1799_5_323 clone-lib P3, complete sequence
cccauaaguuucgucgcaucccagcgacucaucagugggcuaagcccagacuuaugagagagugguuaccucucaggugcccuauucggagcacugcaguucccgauagaaaggcuaagcacuucgcaaugaaguaaggugggacuuuucccuaggguuugagcggucgaacccagggggagugugauccagguaccgccguagaaacuggauuacgacgccuacccgggauucaaacccggucccccucagaggugacucugagugaaaggucugugcuuagcacacugaugaguuccugaaauggaacgaaaccuuucuaa

SEQ ID NO: 273 - RNA, 323 nt, Pelamoviroid
  KF867087.1 Peach latent mosaic viroid clone 703_16_323 clone-lib P7, complete sequence
cccauaaguuucgucgcaucccagcgacucaucagugggcuaagcccagacuuaugagagagugguuaccucucagccccuccaccuuggggugcccuauucggagcacugcaguuucgauagaaaggcuaagcacuucgcaaugaaguaaggugggacuuuucccuaggguuugagcggucgaacccagggggaguguaauccagguaccgccguagaaacuggauuacgacgccuacccggucccccucagaggugacucugagugaaaggucgugcuuagcacacugaugaguuccugaaauggaacgaaaccuuucuaa

SEQ ID NO: 274 - RNA, 323 nt, Pospiviroid
  MF770197.1 Pepper chat fruit viroid isolate TGS17s, partial genome
ucucgaucgacggucgcgucggccuucucgcgcacugcuguccggcuacuacccgguggauacaacugacagaggugcuuuuucuuccacccgacuucuaccgacgcggccgggagugaagcuacccgggacccgagaggaucuccggauucuucuaagggugccuguggugccucccccgaagcccgcuuagggaaaaagaaaggggaagcaagcaucuccuguucagggauccccggggaaaccuggacagaccgggcggagaagcgcacgagcgggaccgucuucugacaggaguaauccccgcugaacagggguuuuca

SEQ ID NO: 275 - RNA, 320 nt, Pelamoviroid
  MK212064.1 Peach latent mosaic viroid isolate y4-pg4, complete genome
acacucauaaguuucgccguaucucaacggcucaucagugggcuuagcccagacuuaugagagaguugugaccucucagccccuccaccuuggggugcccuauccgagcacugcaguuucgguagaaaggcuaagcaccucgcaaugagguaaggugggacuuuuccuucuggaaccaagcgguugguuccgaggggggugugauccagguaccgccguagaaacuggauuacgacgucuacccgggauucaaacccgguccccuccagaagugauugcuaagcacacugaugagucucugaaauaagacgaaacucuug

SEQ ID NO: 276 - RNA, 320 nt, Pospiviroid
  EU273604.1 Potato spindle tuber viroid, partial sequence
  misc_feature (4)..(4): n is a, c, g, or u
  misc_feature (8)..(8): n is a, c, g, or u
  misc_feature (314)..(314): n is a, c, g, or u
ucgngaanaucaggugugaaccacagguaccacgaguuuaguuccgaggaaccucugcgguuccaagggcuuaacucccuccccccgaagccaagagauagagaaaaagccggucucgggagcuucacuugguuccacccgguaauaacccaaacgaccgcgcaaagggggcgaggggugguccugcgggcgcgaggaaggacacccgaacaaaggaagggugaaaaccccgauucuuuuggaauuacuccuguaggccgcugggcacuccccaaaagaccuuauugccaguucgguucagguuuccccguggnauaaaa

SEQ ID NO: 277 - RNA, 319 nt, Apscaviroid
  KY654680.1 Citrus bent leaf viroid isolate PTZ 57R/T, complete genome
guggcgaaguugagcucugcucuucuaagcuguaaccggaccgguccccuucacccgagcgcugcuugccgcuagucgcgcggacuuccaagucucccucccgagccgcuuuucucuucaccuaauuuccguagcagcggggagagggugaagccccugaaccccugagggcuccucggagacuucuugugguuccuguggugucaccccucagcccuaccugcgaaagaaaaaaguguuagaaggcggcagaggagcugacuggucguucgucgacgaaggcucgucagcugcggagguuggggucgacuggccuccg

SEQ ID NO: 278 - RNA, 319 nt, Cocadviroid
  MK795526.1 Hop latent viroid strain CV_7, complete genome
uaaaaagugccccucuggggaauacacuacgugacuuaccuguauggaggcaagggcucgaagagggaucccaggguaaaccuacucgagcgaggcggagaucgagcgccaguucgugcgcggcgaccugaaguugcuucggcuucuucuuguucgcguccugcguggaacggcuccuucuucacaccagccggagauggaaacuacccgguggauacaacucuagagccccgagcuuaaccugcagaagaucacaaaaaaagagccccuauggggaauacacuacgugacuuacguguaugguggcaagggcucgaag

SEQ ID NO: 279 - RNA, 318 nt, Apscaviroid
  MN734784.1 Plum viroid I isolate ZR6, complete genome
  misc_feature (65)..(65): n is a, c, g, or u
uggugaacaaccuugugguuccugugggucaccccccgaccacaaugcuuaaaagaaaaaucagnagagaggaagagacuuaccugucgucgucgacgaaggcugguaagccgaguuggcggaguagcgccgaguggaguaaagacggagaaccucucgaaagagacugacuccgcuuccuccggccgcucccgucuuccgagcuuccugccgcuagucgagcggacaacugguagcucucccacucucucagauuuucucuugagagcgaggucgggagcggaguccccaggggaaaacacgauugguguucuccac

SEQ ID NO: 280 - RNA, 318 nt, Apscaviroid
  M74065.1 Citrus bent leaf viroid RNA
cggagacuucuugugguuccuguggugacaccccucaagcccuaccugcgaaagaaaaaaguguuagaaggcggcagaggagcugacuggucgucgucgacgaaggcucgucagcugcggagguuggggucgacuggcuccgguggcgaaguugagcucugcucuucuaagcuguaacggaccgguccccuucacccgagcgcugcuugccgcuagucgagcggacuuccaagucucccucccgagccgcuuuucuuuucuaccuaauuuccguagcagcggggagagggugaagccccugaaccccugagggcuccu

SEQ ID NO: 281 - RNA, 317 nt, Apscaviroid
  HQ606079.1 Pear blister canker viroid, complete sequence
  misc_feature (172)..(196): n is a, c, g, or u
cuuuccugagguuccuguggugcuccccugaccugcguuccaaaaagcgaaaaagugagaggcccuaggggcuucucggcucgucgucgacgaagggucuagaagccugggcgcuggcuggagcgcgcggcugugaguaaucgcuccuuuggagaagaaaaccagcguugcnnnnnnnnnnnnnnnnnnnnnnnnncccgcuagucgagcggacaacccgagcaccgccgaagcgcuuuuuucuuuuauagcagcuuggcuucggcggcgauggguggaaguuuaccgcggacccccgagaggaggcccucgggucc

SEQ ID NO: 282 - RNA, 317 nt, Apscaviroid
  GU825972.1 Pear blister canker viroid clone PBCV5, complete genome
cuuucccugagguuccuguggugcuccccugaccugcguuccaaaaagcaaaaaaagugagaggccccagggggcuucucggcucgucgucgacgaagggucuagaagccugggucgggcgggagcgcgcggcugugaguaaucgcuccucuggagaagaaaaccagcguugcuucucgccucuuccccgucuucugucccgcuagucgagcggacaacccgagcaccgccgaaggccuuuuuucuuuugaagcagcuuggcuucgcggcgaggguggaaguuuuaccgcggacccccgagaggaggcccucggguc

SEQ ID NO: 283 - RNA, 316 nt, Apscaviroid
  GQ141740.1 Pear blister canker viroid isolate Tzotzis, complete genome
ccuuuccuugagguuccugugguacuccccugaccugcauucccaaaagccaaaaaagugagaggccuuaggggcugcucggacucgucgucgaagaaggggcuagacgcuugugcgcuggcuggagcgcgagccugugaguaauugcuccucuggagaagaaaaccagcguugcuucccgccugagccuguuuuuuugucccgcuagucgagcggacaacccgagccccgccaaaggccuuuuuuuuuuuaagcugcuugguggugcggcgaggguggaagucaccgcggauccccuagaaggaggcccucgggu

SEQ ID NO: 284 - RNA, 316 nt, Apscaviroid
  DQ186640.1 Pear blister canker viroid PBCVd isolate 35, complete genome
cccuuuccugagguuccuguggugcuccccugaccugcguuccaaaaagcgaaaaaagugagaggcccuaggggcuucucggcucgucgucgacgaagggucuagaagccugggcgcuggcuggagcgcgcggcugugaguaaucgcuccuuuggagaagaaaaccagcguugcuuccugccugagccucgucuucugucccgcuagucgagcggacaacccgagcaccgccgaaggccuuuuuucuuuuauagcagcuuggcuucgcggcgaggguggaaguuuaccgcggacccccgagaggaggcccucgggu

SEQ ID NO: 285 - RNA, 316 nt, Apscaviroid
  FJ974082.1 Pear blister canker viroid isolate Leuki-30, complete genome
cuuuccuugagguuccugugguacuccccugaccugcauuccaaaacgcaaaaaaagugagaggcccuaggggcuucucggcucgucgucgacgaagggucuagaagccugggcgcuggcuggagcgcgcggcugugcaguaaucgcuccuuuggagaagaagaccagcguugcuuccugccugagccucgucuucugucccgcuagucgagcggacaacccgagcaccgccgaaggccuuuuuucuuuuaagcugcuugguggugcggcgaggguggaagucaccgcggaucuccuagaaggaggcccucggguc

SEQ ID NO: 286 - RNA, 316 nt, Hostuviroid
  KY508372.1 Hop stunt viroid isolate Xi-AR-Ver, complete genome
uugccccgggagcucuuucucagguaagucuccucccugacuuguuuuuucuuugcuugcccaugcggcaacucgagaauuccccagaggggcucaagagaggauccgcggcagaggcucagauagacaaaaagcagguuggaagacgaaccgagaggugaugccaccggucgcgucucacucgaagagccaggagaagguaaaagaagaagggacgaucgaugguguuucgaagcaagaagccucuacuccagagcaccgcggcccucucuccuacgccucgcgcuggauucugagaagaguggcccccgggcga

SEQ ID NO: 287 - RNA, 315 nt, Apscaviroid
  FJ974088.1 Pear blister canker viroid clone Velistiche, complete genome
ccuuuccuugagguuccugugguacuccccugaccugcauuccaaaacgcaaaaaaagugagaggcccuaggggcuucucggcucgucgucgacgaagggucuagaagccugggcgcuggcuggagcgcgcggcugugaguaaucgcuccuuuggaaaagaaaaccagcggcgauacucgccugagccucguuuuuugucccgcuagucgagcggacaacccgagcaccgccgaaggcccuuuuuucuuuuagcugcuugguggugcggugaggguggaaaucaccgcguaucuccuaaaaggaggcccucgggu

SEQ ID NO: 288 - RNA, 314 nt, Apscaviroid
  EU978464.1 Pear blister canker viroid isolate PEK, complete genome
ccuuuccuugagguuccugugguacuccccugaccugcauuccaaaacgcaaaaaaagugagaggcccuaggggcuucucggacucguugucgaauaagggccuagaagcugggucgggcgggagcgcgcggcugugaguaaucgcuccucuggagaagaaaaccagcguugcuucucgccugagccucguuuuuugucccguuagucgagcggacaacccgagccccgccgaaggccuuuuuuuuuuuaagcugcuugguggugcggcgaggguggaagucaccgcggaucuccuagaaggaggcccucgggu

SEQ ID NO: 289 - RNA, 314 nt, Pospiviroid
  FJ031232.1 Chrysanthemum stunt viroid isolate Beijing, partial sequence
agacaggaguaauccuaaacaggguuuucacccuuccuuuaguuuccuuccucuccuggagaggucuucugcccuagcccggucuucgaagcuuccuuuggcuacuacccgguggaaacaacugaagcuucaacgccuuuuuuuccaaucuucuuuagcaccgggcuagggaguaagcccguggaaccuuaguuuuguucccucgggacuuacuugugguuccuguggugcacuccugacccugcugcuuugaaagaaaaagaaaugaggcgaagaaguccuucagggauccccggggaaaccuggaggaaguc

SEQ ID NO: 290 - RNA, 313 nt, Hostuviroid
  KY508370.1 Hop stunt viroid isolate Xi-MT-Ver, complete genome
uugccccuggucuccuuuuucagguaaguuuccuccccgacuuguuuuuucuuugcuugcccaugcggcaacucgagaauuccccaaaggggcucaagaaaggauccgcggcaaaggcucaaauagacaaaaagcagguuggaagacgaaccgagaggugaugccaccggucgcgucucacucgaagagccaggagaagguaaaaaaagaagggacgaucgaugguguuucgaagcaagaagccucuacuccagagcaccgcggcccucuuccaccccucucgcuggauucugaaaagagugccccgggcgaa

SEQ ID NO: 291 - RNA, 312 nt, Hostuviroid
  KY508369.1 Hop stunt viroid isolate Xi-CA-Ver, complete genome
uuuuccccggggucccuuuucccggaaauuuuuccuccccacuuguuuuuucuuugcuuccccaugcggcaacuccagaauuccccagaggggcucaagaaaggauccgcggcagaagcucaaauagacaaaaagcagguuggaagacgaaccgagaggugaucccaccggucgcgucucacuccaagacccaggaaaagguaaaaaaaaagggacaaccaugggguuucaaaccaaaaacccucacuccaaaccacccgggccucucuccaccccacucccuggguuuugagaaaaaauugccccgggggg

SEQ ID NO: 292 - RNA, 310 nt, Apscaviroid
  KF788291.1 Apple dimple fruit viroid, complete sequence
ggagaaaacuccgugugguuccuguggggcaccccccugcgcuacugacuaaaagaaaaauucaacaggugaagagacuuaccugucgucgucgacgaaggcugguaagccgugaaggcguggaggcggcuccaggucgugccuuuuggaagaaacuccuaaaggacuugacuggaugcccgccugaccugcccgagcuuuccaccgcuagucgagcggacuaauggcaagcucuccgacccuuagaauuucuucuuaggucgggcagggaguggagccuccaggguaaaacacgauugguguuuucccc

SEQ ID NO: 293 - RNA, 309 nt, Hostuviroid
  KJ810551.1 Hop stunt viroid clone MO-D5m, complete genome
cuggggaauucucgaguugccgcaaaaggcaagcaaagaaaaaacaaggcagggaggagacuuaccugagaaaggagccccggggcaacucuucucagaauccggcgagaggcguaggagagagggccgcggugcucuggaguagaggcuucuugcuucgaaacaccaucgaucgucccuucuucuuuuaccuucuccuggcucuucgagugagacgcgaccgguggcaucaccucucgguucgucguccaaccugcuuuuugucuaucugagccucugccgcggauccggauccucucuugagccccu

SEQ ID NO: 294 - RNA, 308 nt, Hostuviroid
  LC507888.1 Hop stunt viroid HSVd-US RNA, complete genome
uugcccggggcaacucuucucagaauccagcgagaggcguggagagagggccgcggugcucuggaguagaggcucugccuucgaaacaccaucgaucgucccuucuucuuuaccuucuucuggcucuucguggagacgcgaccgguggcaccccugcucgguucgcuccaaccugcuuuuguucuaucugcgccucugccgcggauccucucuugagccccucuggggaauucucgaguugccgcaaaaggcaugcaaagaaaaaaacuaggcagggaggaacuuaccugagaaaggagccccggggc

SEQ ID NO: 295 - RNA, 307 nt, Apscaviroid
  MG516817.1 Apple dimple fruit viroid isolate IRN9, complete genome
ggaggaaaacuccgugugguuccuguggggcaccccccugcgcaaaugacuaaaagaaaaaucagcaggugaagagacuuaccugucgucgucaacgaaggcugguaagccgugacgcguggaggagagcccgggucgcgcucuuggggguaaccccuuugagacuugaccgguuccucucccacccugccgagcuuccugccgcuagucgagcggacuccggguuagcucuccgacccucagauuuucucuuaaggucuggcagggaguggagcucccaggguaaaacacgauugguguuuucccc

SEQ ID NO: 296 - RNA, 306 nt, Apscaviroid
  MG516811.1 Apple dimple fruit viroid isolate IRN3, complete genome
ggaggaaaacuccgugugguuccuguggggcaccccccugcgccaaugacuuaaagaaaaauccgcaggugaagagacuuaccuguccuccuccaacaaaggugguaaacccugaccccuggaagaaaacccgggucccgcucuuggggguaaccccuuugagaacugaccgguccuuuucccacccugccgagcuuccugccguuaguggagcggauuccggguugcucuccgacccucagauuuucucuuaaggucuggcagggaguggagcucccaggguaaaacacgauugguguuuucccc

SEQ ID NO: 297 - RNA, 306 nt, Apscaviroid
  KX909575.1 Apple dimple fruit viroid isolate IRN1, complete sequence
ggaggaaaacuccgugugguuccuguggggcaccccccugcgcaaaugacuuaaagaaaaauccgcaggggaagaaacuuaccuguccuccucgaggaagcuggguaagccgugacgcguggaggagggcccgggucgcguucuuggggguuaccccuuugagacuugaccgguucuuuucccacccugccgagcuuccugccguuaguggagcggauuccggguagcucuccgacccuccgauuuucucuuaaggucugggagggaguggagcucccaggguaaaacacgauugguguuuucccc

SEQ ID NO: 298 - RNA, 306 nt, Hostuviroid
  MH476218.1 Hop stunt viroid, complete genome
aacccggggcaacucuucucagaauccagcgagaggcguggagagagggccgcggugcucuggaguagaggcucugccuucgaaacaccaucgaucgucccuucuucuuuaccuucuucuggcucuuccgaugagacgcgaccgguggcaucaccucucgguucgucccaaccugcuuuuugucuaucugagccucugccgcgauccucucuugagccccucuggggaauucucgaguugccgcaucaggcaagcaaagaaaaaacaaggcagggagguacuuaccugagaaaggagccccggguu

SEQ ID NO: 299 - RNA, 306 nt, Hostuviroid
  JQ080274.1 Hop stunt viroid isolate HSVdr, complete genome
cuggggaauucucugcacguugccgcacaaggcaagcaaagaaaaaaacaaggcagggaggagcacuuaccugagaaaggagccccggggcaacucuucucagaauccagcggaggcguggagagagggccgcggugcucuggaguagaggcucugucuucgaaacaccaucgaucgucccuucuucuuuaccuuccuucggcucuucgagugagacgcgaccgguggcaccccuccuggguucgcucuccacuccugcuuuuuguucuaucugagccucugccgcggauccucucuugagccccu

SEQ ID NO: 300 - RNA, 304 nt, Apscaviroid
  NC_035620.1 Lychee viroid-like RNA isolate 20160105, complete sequence
cuggaaacaaccuugugguuccugugggucaccccuccaaagcaauaaaggaaaagaaaggcgaagacucaccugucgucgucgacgaaggcagcugagccaacuucgaugaagcuggcgggagacgcgcugagaagagaaaagcucccuccggggagcgcuuuuaaaacucucaaagcgcuuccccgcuccgcugagaagacgguccaguggccgcuagucgagcggacaaagguucgucucgcccuaaauuccugcagcggagagcggcguccccaggguaaaacacgauugguguuuucca

SEQ ID NO: 301 - RNA, 303 nt, Apscaviroid
  LC214530.1 Apple dimple fruit viroid genomic RNA, complete genome, isolate: ADFVd-J, clone: JG16-1
ggaaaacuuccgugugguuccuguggggcacccccccgcgcagugcuuuaaaagaaaauucaguaggugaagagacuuaccugucgucgucgacgaaggcugguaagccgacgcguggauggggacccgggucgcgcucuuggaggaaacuccuuugagacuugaccggcgagccuucuccugcagagcuuuccugccgcuagucgagcggacaacgggcagcucuccgacccucagaauuucucuucaggccgggcggggaguggagcucccaggguaaaacauacaaucgguguuuucccc

SEQ ID NO: 302 - RNA, 303 nt, Hostuviroid
  MH537090.1 Hop stunt viroid isolate SC-20, complete genome
cuggggaagauucucgaguugccgcaaaaggcaugcaaagaaaaaaacuaggcagggaggcgcuuaccugagaaaggagccccggggcaacucuucucagaauccagcgagaggcguggagagagggccgcggugcucuggaguagaaaggcucugccuucgaaacaccaucgaucgucccuucuucuuuaccuucuucugacucuucuuggagacgcgaccgguggccgaccccugcucgguucgcuccaaccugcuuuuguucuaucugcgccucugccgcggauccucucuugagccccu

SEQ ID NO: 303 - RNA, 303 nt, Hostuviroid
  MF576418.1 Hop stunt viroid isolate DgHV-3, complete genome
cuggggaauucucgaguugccgcauaaggcaugcaaagaaaaaaacuaggcagggaaggugcuuaccugagaaaggagccccggggcaacucuucucagaauccagcgagaggcguggagagagggccgcggugcucuggaguagaggcucugccuucgaaacaccaucgaucgucccuucuucuuuaccuucuucuggcucuuccgauggagacgcgaccgguggcaucaccucucgguucgucccaaccugcuuuuuguugucuaucugagccucugccgcggauccucucuugagccccu

SEQ ID NO: 304 - RNA, 302 nt, Hostuviroid
  KT000360.1 Hop stunt viroid isolate 22_2_3, complete genome
  misc_feature (28)..(28): n is a, c, g, or u
  misc_feature (51)..(59): n is a, c, g, or u
  misc_feature (148)..(150): n is a, c, g, or u
  misc_feature (154)..(154): n is a, c, g, or u
  misc_feature (207)..(209): n is a, c, g, or u
  misc_feature (247)..(247): n is a, c, g, or u
  misc_feature (249)..(249): n is a, c, g, or u
cuggggaauucucgaguugccgcauggncaagcaaagaaaaaacaaggcannnnnnnnncuuaccugagaaaggagccccggggcaacucuucucagaauccagcgagaggcguaggagagagggccgcggugcucuggaguagaggnnncuuncuucgaaacaccaucgaucgucccuucuucuuuuaccuucuucuggcucuucnnnugagacgcgaccgguggcaucaccucucgguucguccncnaaccugcuuuuugucuaucugagccucugccgcggauccucucuugagccccu

SEQ ID NO: 305 - RNA, 302 nt, Hostuviroid
  KU640955.1 Hop stunt viroid isolate HSVd-Mor-ValL_Big, complete sequence
cuggggaauucucgaguugccgcauggccaagcaaagaaaaaacuaggcagggaccagacuugcuugaaaaagcagccccggggcaucucuucucugaauccuucgagaggcguaggcgagagggccggguugcucuggaguagaggcuucuugcuucgaaacaucaucguucgucccuucuucuuuuaccuucuccuggcucuuccgaugagacgcgaccgguggcaucaccucucgguucgucuuccaaccugcuuuuugucuauccaagccucugccgcgguuucucucuugagccccu

SEQ ID NO: 306 - RNA, 302 nt, Hostuviroid
  KP126952.1 Hop stunt viroid clone HSVd-F1, complete genome
cuggggaauucucgaguugccgcaugggcaagcaaagaaaaaacaagggcagggaggagacuuaccugagaaaggagccccggggcaacucuucucagaauccagcgagaggcgucggacggggggccgcggugcucuggaguagaggcuucuagcuucgaaacaccaucgaucgucccuucuucuuuaccuucuacuggcucuucuguugagacgcgaccgguggcaucaccucucgguucgcucccaaccugcuuuuugucuauucugagccucugccgcggauccucucuugagccccu

SEQ ID NO: 307 - RNA, 302 nt, Hostuviroid
  FJ626867.1 Hop stunt viroid isolate e, complete genome
cuggggaauucucgaguugccgcacaggcaagcaaagaaaaaaaaagggagggagaagaacuuaccugagaaaggagccccggggcaacucuucucagaauccagcgagaggcguaggagagagggccgcggugcucuggaguagaggcuucuagcuuucaaaacaccaucaaucgucccuucuucuucccuuucaacggguucuucgguugaaacccaaccgggggcucccccucucgguuccuccccaaccgguuuuuugccuuucugagccucugccgcggauccucucuugagccccu

SEQ ID NO: 308 - RNA, 301 nt, Coleviroid
  NC_003682.1 Coleus blumei viroid 2, complete genome
cggaucauuuucuugugguuccugugguacaccuccgaaggcgcgcuaggcgagaagagaguaaauagaggagagagcuuaccuggguucccuggcagcgcugcaacggaauccagugcccaccggagcgccaguucgugcgagagccgcuucggcuucuucuucgcggaacggcuccgcguguacgccugggaccccgcgagcgauugcaaaggucccgguucgcucuccuuuucuuuuuuaccuacuucagccgaccgccccggggagugaaucacccgggaccgcgaaaagcgauccu

SEQ ID NO: 309 - RNA, 300 nt, Hostuviroid
  AY513267.1 Citrus cachexia viroid, complete genome
gauccucucuugagccccucuggggaauucucgaguugccgcaugggcaagcaaagaaaaaaacaaggcagggaggagacuuaccugagaaaggagccccggggcaacucuucucagaauccagcgggggcguggagagagggccgcggugcucuggaguagaggcuucugcuucgaaacaccaucgaucgucccuucuucuuuaccuccuucuggcucuucgagugagacgcgaccgguggcaaucaccucucgguucgucuuccaaccugcuuuuugucuaucugagccucugccgcg

SEQ ID NO: 310 - RNA, 299 nt, Hostuviroid
  KM213398.1 Hop stunt viroid isolate HR174339, complete sequence
cuggggaauucucgaguugccgcaucaggcaagcaaagaaaaaacaaggcagggagguacuuaccugagaaaggagccccggggcacgacucuucucagaauccauaccuagagaggugcgcacggcggaggugcucuggauacaagcucugccguucgaaacaccaucgaucgugccuucuucuuuaccuucuucuggcucuuccgaugagacgcgaccgguggcaucaccucucgguucgucccaaccugcuuuuugucuaucugagccucugccgcggauccucucuugagccccu

SEQ ID NO: 311 - RNA, 299 nt, Hostuviroid
  GQ260206.1 Hop stunt viroid isolate IIbI1, complete genome
cuggggaauucucgaguugccgcauaaggcaagcaaagaaaaaacaagggcaggagagacuuaccugagaaaggagccccggggcaacucuucucagaauccagcgggggcguggagagagggccgcggugcucugggguagaggcuucuagcuucgaaacaccaucgaucgucccuucuucuuuaccuccuucuggcucuuccgaugagacgcgaccgguggcaucaccuccuagguuucgucaaaccugccuuuuugucuaucugagccucugccgcggauccucucuugagccccu

SEQ ID NO: 312 - RNA, 298 nt, Apscaviroid
  EU382205.1 Citrus viroid III isolate CQ clone 1, complete genome
ggaggaaacuccgugugguuccuguggggcacacccccuugccgaaaauaaaacgcagagagggaaagggaaacuuaccugucgucgucgacgaaggcagcuaaguuggugacgccgaguggaguaaagacggagaguaguucuccgcuagucggaaagacuccgcauccuccggcagacccuucuagcucccgcuagucgagcggacaacugagugaguugucccaauccuaaucuguuuuuaucuaggcuagaaggggauugggccuccaggguaaaacacgauugguguuucccc

SEQ ID NO: 313 - RNA, 298 nt, Hostuviroid
  KP126944.1 Hop stunt viroid clone HSVd-F8, complete genome
cuggggaauucucgaguugccgcaugaggcaagcaaagaaaaaacuaggcagggaggcgcuuaccugagaaaggagccccggggcaaccucuucucagaauccagcgugaggcguaggagagagggccgcggugcucuggaguagaggcuucugcuucgaaacaccaucgaucgucccugcuucuuuuaccuuccuggcucuucgagugagacgcgaccgguggcaccccugcuggguucgcuccaccugcuuuuuguucuaucugcgccucugccgcggauccucucuugagccccu

SEQ ID NO: 314 - RNA, 297 nt, Apscaviroid
  KY110718.1 Citrus dwarfing viroid strain R140910-12, complete genome
gaaacuccgugugguuccuguggggcacacccccuugccgaaaauaaaaugcagagaggggaaagggaacuuaccugucgucgucgacgaaggcagcuaaguuggugaagccgaguggaguaaagacggagaaccuccgcuagucggaaagacuccgcuuccuccggcugacccuccuucagcucgccgcuagucgagcggacaacugagugaguagccccaacccuaaucuguuuuuaucuaggcuagaaggggauugggccuccaggguaaaacacgauugguguuuccccggag

SEQ ID NO: 315 - RNA, 297 nt, Apscaviroid
  GQ260210.1 Citrus dwarfing viroid isolate IIIaI2, complete genome
ggaggaaacuccgugugguuccuguggggcacaccucccggccgaaaauaaaaugcagagagggaaaagggaacuuaccugucgucgucgacgaaggcagcuaaguuggugacgccgaguggaguaaagacggagagucuccgcuagucggaaagacuccgcuuccuccggcugacccuccuucagcucgccgcuagucgagcggacaacugagugaguagccccaauccuaaucuguuuuuaucuaggccugcgggagauugggccuccaggguaaaacacgauugguguuucccc

SEQ ID NO: 316 - RNA, 297 nt, Cocadviroid
  KT923143.1 Coconut cadang-cadang viroid, complete genome
cuggggaaaucuacagggcaccccaaaaacuacugcaggagaggccgcuugagggauccccggggaaaccucaaacgaaucugggaagggagcguaccugggucguucgugcgcguuggaggagcugggucgaucgugcgcguuggaggagacuccuucguagcuucgacgcccgccuccuucguagcuucgacgcccggccgccccuccucgaccgcuugggagacuacccgguggauacaacucacgcggcucuuaccuguuguuaguaaaaaaaggugucccuuuguagccccu

SEQ ID NO: 317 - RNA, 297 nt, Hostuviroid
  KF007324.1 Hop stunt viroid isolate 6931_HSVd_Char, complete genome
uuggggaauucucgaguugccgcaaaaggcaugcaaagaaaaaacacgacagggaagggcuuaccugagaaaagagccccggggcaagucuuuucugaauccagcgagaggcguggagagagggccgcggugcucuggaguagagggugugccuucgaaacaccaucgaucgucccuucuuuuuuaccuucuuuuggcucuuccgaugagacgcgaccgguggcaucaccucucgguucgucccaaccugcuuuuguucuauuugagccuuugccgcggauccucucuugagccccu

SEQ ID NO: 318 - RNA, 297 nt, Hostuviroid
  KC677729.1 Hop stunt viroid isolate 332, complete sequence
cuggggaauucucgaguugccgcaucaggcaugcaaugaaaaaacuuggcagggagguacuuaccugaaaaaggagccccggggcaacucuucucagaauccagcgagaggcguggagagagggccgcggugcucuggaguagaggcuucugcuucgaaacaccaucgaucgucccuucuucuuuaccuuccuucuggcucuucguggagacgcgaccgguggcaccccugcucgguucgcuccaaccuguuuuuugucuaucugauccucugccgcggauccucucuugacccccu

SEQ ID NO: 319 - RNA, 297 nt, Hostuviroid
  FJ974079.1 Hop stunt viroid isolate Kernitsa-327, complete genome
cuggggaauucucgaguugccgcaucaggcaagcaaagaaaaaacaaggcagggagguacuuaccugaaaaaggagccccggggcaacucuucucagaauccagcgagaggcguggagagagggccagcggugcucuggaauacaggcucugcuuuggaccacaaucuuucgucccuucuucuuuaccuucuacuggcucuuccgaugagacgcgaccgguggcaucaccucucgguucgucccaaccugcuuuuugucuaucugagccucugccgcggauccucucuugagccccu

SEQ ID NO: 320 - RNA, 297 nt, Hostuviroid
  DQ444476.1 Hop stunt viroid isolate HNiagD08, complete genome
cuggggaauucucgaguugccgcauccggcaagcaaagaaaaaacaaggcagggaggugcuuaccugagaaaggagccccggggcaacucuucucagaauccagcgagaggcguggagagagggcggcgguggugcggacuagaggcucugcagugcaaacaccaucgaucgucccuucuucuuuaccuucuucuggaucuuccgacgagacgugaccgguggagugaaguuucgguucaucccaaccugcuuuuugucuaucugagcaugugcagcggauccugucuugagccccu

SEQ ID NO: 321 - RNA, 296 nt, Apscaviroid
  LC500205.1 Hop stunt viroid CB RNA, complete genome
cuggggaauucucgaguugccgcacaaggcauggaaagaaaaagaauagggagcugaagacuuaccuggagagaggauccccggggcaacucuuuccagaauccagggggcuccgagcgcaacgggccgcggugcucuggaguagaggcucugcccucgaaacaccaucgaucgucccugcagccuucgucccuccggcucuucuguugagacgcgaccgguggaaaccuuaccaguucgucuaagcuccuuuuuuccuaucgaucccucugccgcggacccucucuugagccccu

SEQ ID NO: 322 - RNA, 296 nt, Apscaviroid
  KT725632.1 Citrus dwarfing viroid isolate NRCV04, complete sequence
ggaaaacuccgugugguuccuguggggcacacccccuugccgaaaauaaaacgcagagagggaaaagggagcuuaccugucgucgucgacgaaggcagcuaaguuggugacgccgaguggaguaaagacggagagucuccgcuagucggaaagacuccgcauccuccggcugacccuccuucagcucgccgcuagucgaguggacaacugagugaguagccccaauccuaaucuauuuuugucuaggcuagaaggggauugggccuccaggguaaaacacgauugguguuuucccc

SEQ ID NO: 323 - RNA, 296 nt, Hostuviroid
  AF100641.1 Hop stunt viroid strain Kh, complete genome
gaccccuuaagagcucaacggcguuuuccguucguuucuuuuuuguuccguccuuccaugaauggacucuuuccucggggccccguugagaagagucuuaggucgcucuccgcaccucucucccggcgccacgagaccucaucuccgagacggaagcuuugugguagcuagcagggaagaagaaauggaagaagaccgagaaggcuacucugcgcuggccaccguaguggagagccaagcaggguuggacgaaaaacagauagacucggagacggcgccuuggagagaacucggga

SEQ ID NO: 324 - RNA, 295 nt, Coleviroid
  NC_003882.1 Coleus blumei viroid, complete genome
uggcucgaacugacuagaacgguuuaaagaaaaaguucuuaccuggaaccuggguucccuggcagcgcugcaacggaauucagggcguuggccccgauggaguuaccgaugagcaccggugagaagagacugucucgcagacuagaagucucuucuuuucugcuuugagacuucuucuccguaggacucuucggcucuucgcggguacccagcaaccugcucccacgcggcgauugcaaaggauccgguccgguucgaaccucuuuucuaaacccuaguucagcuuaaacgagcu

SEQ ID NO: 325 - RNA, 294 nt, Apscaviroid
  MN640603.1 Citrus viroid V isolate F19, complete genome
ugggugaacaaccuugcgguuccugugggucaccccgcccucaggaauaauaaaagcagaggaguagaaaguacucaccugucgucgucgacgaaggccggugagcaguaagccggacgguccccucgcggccguuccucuggagcucugcucuaagaacuucgucgcugaggcccgcgccgccgcucuuccgcgcugcuagucgagcggacguugguagucucccucucccugugcaauaaaauccuagcggcgagugguguccccaggguaaaacacgauugguguucuccc

SEQ ID NO: 326 - RNA, 294 nt, Apscaviroid
  MF477874.1 Citrus viroid V isolate CVd-V-VC-8, complete genome
ugggugaacaaccuugugguuccugugggucaccccgccccacggaauaauaaaagcagaggagaagaaaguacucaccugucgucgucgacgaaggccggugagcaguaagccggacgguccccucgcggccgauccucuggagcucugcucuaagaucuucgcugcugaggcccgcgccgccgcucuuccgcgcugcuagucgagcggacguugguggucuuccucucccugugcaauaaaacccagguggcgagugguguccccaggguaaaacacgauugguguucuccc

SEQ ID NO: 327 - RNA, 294 nt, Apscaviroid
  MF421247.1 Citrus dwarfing viroid clone CV18, complete genome
ggaggaaacuccgugugguuccuguggggcacacccccuugccgaaaauaaaaugcagagaggggaaagggaacuuaccugucgucgucgacgaaggcagcuaaguuggugacgcacuguggaauucgcccuugucuccgcuagucggaaagacuccgcauccuccggcugacccuccuucagcucgccgcuagucgagcggacaacugagugaguagccccaacccuaaucuguuuuuaucuaggcuagaaggggauugggccuccaggguaaaacacgauugguguuucccc

SEQ ID NO: 328 - RNA, 294 nt, Apscaviroid
  KC460712.1 Citrus viroid V isolate WN4R, complete genome
ugggugaacaaccuucccgguuccugugggucaccccgccccacggaauaauaaaagcagaggaguagagaguacucaccugucgucgguagagaguacucaccugucgucgguaaagaguacuccccugucgucggaagagaguaucugcugucgauggucgauggugaggccugcgccgccgcucuugaaaaacugaaagucgagcggacguugguggucucccucucccugugcaauaaaauccagguggcgagugguguccccaggguaaaacacgauugguguucuccc

SEQ ID NO: 329 - RNA, 294 nt, Cocadviroid
  MK795572.1 Hop latent viroid strain CV_53, complete genome
gugaacuucugcagguuaagcucggcgcucaagaguuguauccaccggguaguuuccaacuccgucugguguuaagaaggagccguuccacgcaggacgcgaacaagaagacgccgacgcaacuucaggucgccgcgcacgaacuggcgcucgaucuccgccucgcucgcguagggcuccccggggaucccucuucgagcccuugccaccauacagguaagucccguaguguauuccccagcggggcacuuucuaugagaacuucugcaggccccgcccggcgcucaagcgucg

SEQ ID NO: 330 - RNA, 293 nt, Apscaviroid
  KY110720.1 Citrus viroid V strain R141013-19, complete genome
gucaccccgccccacgggaaauaaaagcagaggagaagaaaguacucaccugucgucgucgacgaaggccggugagcaguaagccggacgguccccucgcggccgauccucuggagcucugcucuaagaucuucgcugcugaggcccgcgccgccgcucuuccgcgcugcuagucgagcggacguugguggucuuucucucccugugcaauaaaacccaagcggcgagugguguccccaggguaaaacacgauugguguucucccugggugaacaaccuugugguuccugugg

SEQ ID NO: 331 - RNA, 293 nt, Apscaviroid
  KC460711.1 Citrus viroid V isolate TN1R, complete genome
ugggugaacaaccuugugguuccugugggucaccccgccccacggaauaauaaaagcagaggaguagagaguacucaccugucgucgguagagaguacucaccugucuucgguagagaguacucaccugucgucgguagacaguacucaccugcugucgaccgucgaugcugacgccuguccgccgcucuuccgcucugcugucgagcggaauugguggucucccucucccugugcaauaaaauccagguggcgagugguguccccaggguaaaacacgauugguguucuccc

SEQ ID NO: 332 - RNA, 293 nt, Apscaviroid
  JQ072089.1 Citrus viroid V from Citrus limon, complete genome
ugggugaacaaccuugugguuccugugggucaccccgccccacggaauaauaaaagcagaggaguagagaguacucaccugucgucgcucgacgaaggccggugagcagaaggcuuagcggauccccucgcggcaagacgcccggaaccgcgauaaagaacucgccgccgagcccgcgccgccgcuccaccgcgcugcuagucgagcggacgccgguggucucccucucccugugcaauaaaauccagguggcgagugguguccccaggguaaaacacgauugguguucuccc

SEQ ID NO: 333 - RNA, 292 nt, Cocadviroid
  MK795541.1 Hop latent viroid strain CV_22, complete genome
aaaaagugccccucuggggaauacacaacguuacuuacuugaauguugcaaagggcucgaagagggauccccggggaaaccuacucgagcgaggcggagaucgagcgccaguucgugcgcggcgaccugaaguugcuucggcuucuucuaguucgcgaccugcguggaacggcuccuucuucacaccagccggagaucgaaacuacccgguggauacaacucuugagcgccgagcuuaaccugcagaaguucacauaaaaagugcauauaguaagagcuuaguguagggaaa

SEQ ID NO: 334 - RNA, 292 nt, Cocadviroid
  MK795539.1 Hop latent viroid strain CV_20, complete genome
acuucugcagguuaagcucggcgcucaagaguuguauccaccgaguaguuuccaacuccggcuggugugaagaaggagccguuccacgcaggacgcgaacaagaagaagccgaagcaacuucaggucgccgcgcacguucuggcgcucgaucuccgccucgcucgaguagguuuccccggggaucccucuucgagcccucgccaccauacagguaagucacguaguguauuccccagaggggcacuuuuuaugugaacuacugcaggucacgcucggcgcucaagagucgca

SEQ ID NO: 335 - RNA, 289 nt, Apscaviroid
  MF421250.1 Citrus dwarfing viroid clone CV32, complete genome
cacuguggaauucgcccuucgucaccaacuuagcugccuucgucgacgacgacagguaaguuucccuuucccucucugcguuuuauuuucggcaagggggugugccccacaggaaccacacggaguuuccuccggggaaacaccaaucguguuuuacccuggaggcccaauccccuucuagccuagauaaaaacagguuaggauugggacaacucacucaguuguccgcucgacuagcgggagcuagaagggucugccggaggaugcggagucuuuccgacuagcggag

SEQ ID NO: 336 - RNA, 289 nt, Apscaviroid
  KY473929.1 Citrus dwarfing viroid isolate En-SR-Ver, complete genome
uuucgucgacgacgacagguaaguuuccaguacccgauaugcgaucuguugcggcagggggagugccaaaaggaaccacacggaguuuccuccggggaaacaccaaucguguuuacccuggacgcccaauccccuucuagccuacauaaaaacagauuaggauugggacacucacucaguuguccgcucgacuagcgggagcuacaagggucugccggaggaugcgagccacgacaacuagcagagacucuccaucaguacuccacuccgcgucugcccuuacugccaa

SEQ ID NO: 337 - RNA, 287 nt, Cocadviroid
  MK795563.1 Hop latent viroid strain CV_44, complete genome
uuccgaucugcagguuaagcucggcgcucaugaguuguauccaccggguaguuuccaacugcggcuggugugaagaaggagccguuccacgcaggacgcgaucaagaagaagccgaagcaacuucagggcgccgcgcacgaacuggcgcucgaucuccgcgucgcuggaguagguuuccccgggguucccucuucgagcccuuggcaccauacauguaagucauguaguguauuccccagaggggcacauuuuaugugaacuucugcaggguaagcucggcgcucaa

SEQ ID NO: 338 - RNA, 287 nt, Cocadviroid
  MF421255.1 Citrus bark cracking viroid clone CC23, complete genome
cuggggaauuucucugcgggaccgaauaaaaacagcuugcggagggaacauaccugaagagggaucccccggggaaaucucuucagacucgucgaggggagggcgccgcggaucacuggcguccagcaccggaaacaggagcucgucuccuuccuuuccaccgcuggcuccacauccgaucgucgcuucuuccuucgcgaccugagauagaaacuaccugguggauacaacucuuggguuguuccucccaggcuuguuaaauaaaauggcccgcguuugagaccccu

SEQ ID NO: 339 - RNA, 285 nt, Cocadviroid
  MK795635.1 Hop latent viroid strain CV_116, complete genome
aaacuucccgguggauucaacucuugagcgccgagcuuaaccugcagaaguucacauaaaaagugcccgucuggggaagacacuacgugacugaccuguaugguggcaagggcgcgaacagggauccccggggaaaccuacucgagcgaggcggagaucgagcgccauuucgugcgcggcgaccugaaguugcuucggcugcuucuuguucgcguccugcguggaacggcuccuucuucaccccagccggagauggaagcuacccggaggauacaacucgugagc

SEQ ID NO: 340 - RNA, 285 nt, Cocadviroid
  MK795520.1 Hop latent viroid strain CV_1, complete genome
cgacucuugggcgccgggcguggccgggagaaguucucuuaaagaguggcccgcuggggaauacacuacguggcuuaccuguaugggggcaagggcucgaagagggauccccguggaaagcuacucgagcgaggcggagaucgagcgccaguucgagcccggcgaccugaaguugcuucggcuucuucuuguucgcguccugccugcaacggcuccuucuucacaccagccggaguugaaaaauacccgguggauacaacucuugagcgccgagcuuaaccugcc

SEQ ID NO: 341 - RNA, 284 nt, Cocadviroid
  MK795629.1 Hop latent viroid strain CV_110, complete genome
uccccagaggggcacuuuuuaugugaacuucuggagguuaugcucggcgcucaagaguuguaucccccggguaguuuccaacugcggcuggugugaagaaugagccguuccaagcaggacgcgaacaagaagaagccgaaggaacuucaggucgccgcgcacgaacuggcgcucgaucuccgucucgcucgaguagguuuccccggggaucccucuucgagcccuugccaccauacagguaagccacgaaguguauuccccagaggggcacuuuuuaugcgaac

SEQ ID NO: 342 - RNA, 284 nt, Cocadviroid
  MK795551.1 Hop latent viroid strain CV_32, complete genome
aacggcuccuucuucacaccagccugaguuggaaacuacccguuggauacaacucuugaucgccgagcuuaaccugcagaaguucacauaaaaagugacccucugggcaauacacuacgugacuuaccuguaugguggcaagggcucgaagagggauccccggggaaaccuacucgagcgaggcggagaucgagcgccugugcgugcgcggcgaccugaaguugcuucggcuucuucuaguuagcguccagaguggaacggcuccuucuucacaccacccggag

SEQ ID NO: 343 - RNA, 283 nt, Cocadviroid
  MF198463.1 Citrus bark cracking viroid isolate W11, complete genome
cuggggaauuuucucacugggaccuaaaaaaacugcucgaggaggguacuuaccugaagagggauccccggggaaaucucuucagaccuucgaggggagggcgccgcggaucacuggcgugccggcucggaaacaggaguuccgacuccuuccuuucucugccgcgccuccacauccgaucgucgcuucuccuuucggaccugaaagaaacuacccgguggauacaacucuugggucguaccucccgggcuuguuaauaaaaggcccagucuagagaaccccu

SEQ ID NO: 344 - RNA, 281 nt, Cocadviroid
  MK795534.1 Hop latent viroid strain CV_15, complete genome
uguucucuucagaucuucuugagcgcagagcuuaaccugcagaaguucacauaaaaauugccccucuguggaauacacuacguuacuuaccuuuaugguggcaagggcucgaagagggauccacggggaaaccuacucgagcgaggcggagaucgagcgccaguucuugcgcggcgaacugaaguugcuucggcuucuucuuguucgcguccugcguggaacgucuccuucuucacaccagccggaguuggaaacuacccggaggauacaacucuugaccc

SEQ ID NO: 345 - RNA, 281 nt, Hostuviroid
  JX945670.1 Hop stunt viroid isolate 5018, partial sequence
uggcaauuguucucagaauccagcgagaggcguaggagagagggccgcggugcucuggaguagaggcuucuugcuucgaaacaccauccaucguccguucuucuuuuaccuucuccuggcucuucgarugagacgcsaccgguggcaucaccucucgguucgucuuccaaccugcuuuuuggcuaucugagccucugccgcggauccucucuugagccccucuggggaauucucgacuugccgcaugggcaagcaaaaaaaaagcaaggcaagaaugacac

SEQ ID NO: 346 - RNA, 277 nt, Cocadviroid
  MK795523.1 Hop latent viroid strain CV_4, complete genome
agaguuguuuccaccggguaguuuccaacuccgucuugugugaagaaggagccguuccacgcaggacgcgaacaagaagaagccgaagcaacuucagggcgccgcgcacgaacuggcgcucgaucuccgccuggcucguguagguuuccccggggaucccucuucgagcccuugccaccaucccgguaagucacguaggguauuccccagaggggcacuuuuuaugugaacuucugcagguuaagcucggcgcucaagaguuguauccaccggguag

SEQ ID NO: 347 - RNA, 277 nt, Cocadviroid
  MG457782.1 Citrus bark cracking viroid isolate P2-1, complete genome
cuggggaauuucucugcgggaccaaauaaaaacagcuugaggagggaacuaaccugaagagggauccccggggaaaucucuucagaccguugaggggagggcgccgcggaucacuggcgagcagaaacaggagcucgucuccuuccuuucuugccuccacauccgaucgucgcuucucucuucucgaccugaaaaagaaacuacccgguggauacaacucuuugaagguuguuccucccaggcuuguuaaauaaaauggcccgccuuagagaccccu

SEQ ID NO: 348 - RNA, 277 nt, Hostuviroid
  JX401927.1 Hop stunt viroid isolate 599, complete genome
cuggggaauucucgaguugccgcaaaaggcaugcaaagaaaaaaacuaggcagggaggcgcuuaccugaaaaaggagccccggggcaacgcucuucucagaaucccaaagggggggccccccaagauacacguugcgaaacaccauuuaccgucccuucuucuuuaccuucauucuggcucuucguggagacgcgaccgguggcaccccugcucgguucgcuccaaccugcuuuuguucuaucugcgccucugccgcggauccucucuugagccccu

SEQ ID NO: 349 - RNA, 276 nt, Cocadviroid
  MK795637.1 Hop latent viroid strain CV_118, complete genome
cgcagcgcggcgcgcaagaguuguaucccccgggucguguccaacuccggcuggugugaagaaggagccguuccacgcaggacgcgaagaagaaguagccgaagcaacuucaggucgccgcgcacgaacuggcgcgcgaucuccgccgcgcucgaguaggguuccccggggaucccucuucgagcccuugccaccauacagguaagucacguaguguauucccccgcggggcccguuuuaugugaacuucugccggguacgcgcggcgcgccagcg

SEQ ID NO: 350 - RNA, 276 nt, Cocadviroid
  MK795631.1 Hop latent viroid strain CV_112, complete genome
aacuucugcagguuaugcucggcgcucaagaguuguauccaccggguaguuuccaacuccggcuggugugaagaaggagccguuccacgcaggacgcgaacaacacgaagccgaagcaacuucaggucgccgcgcacgaacuggcgcucgaacucccccucgcucgaguagguuuccccggggaucccucuacgagcccuugccaccaaacaggaaugucacguagucuauuccccagagggggacuuuauaucugaacuucugcagguuaagcac

SEQ ID NO: 351 - RNA, 275 nt, Cocadviroid
  MK795580.1 Hop latent viroid strain CV_61, complete genome
guaagcgcggcgcgcaaguguuguauccaccggguagguuccaacuccggcuggugggaagcaggagccguuccacgcaggacgcgaacaagaagaagccgaagcaacuugaggucgccgcgcacgaacuggcgcucgaucuccgccucgcucgaguagguuuccccggggaucccgcuucgagcccuugccaccauacagguaauucccguaggguauuccccagagggucacuuuuuaugugaacuucugcauguucagcucggcgcucaaga

SEQ ID NO: 352 - RNA, 274 nt, Coleviroid
  MG767213.1 Coleus blumei viroid 5 clone 8, complete sequence
ugaccucaauugacuagaacaguaguaaagaaaaggcagguugcuuaccuggguucccuggcagcgcugcaacggaauucagggcacggagucggugccagaucgugcggcuucggcgccaguucgugcgagagcuucgcucgcgaacucuacguggaacggccgucgcucguggaucggccgucuuccggguuccugcucccacgcggcgauugcaaaggagcugguucgcguccuguccuauucuucuacucuaguucaacuuaaagagguu

SEQ ID NO: 353 - RNA, 273 nt, Cocadviroid
  MK795586.1 Hop latent viroid strain CV_67, complete genome
aucuggguaguuuccaacuccggcuggugugaagaaggagccguuccacgcaggacgcgaacaagacgaagccgaagcaacuucagggcgccgcgcacgaaguggcgcucgagcuccgccucgcucgagucgguuuccccggggaucccucuucgagccccugccaccauacaggaaagucacguaguguauuccccagaugggcacuuuuuaugugaaauucuucagguuaagaucagagauauagagcuguaugcucggguuuguuuccau

SEQ ID NO: 354 - RNA, 272 nt, Apscaviroid
  MN379511.1 Citrus dwarfing viroid isolate Hawaii-3, partial genome
cuucaccaacuuagcugccuucgucgacgacgacagguaaguucccuuuccccucucugcauuuuauuuucggcgaggaggugugccccacaggaaccacacgauuguuccuccggggaaacaccaaucguguuuuacccuggaggcccaauccccuucuagccuagauaaaaacagauuaggauugggacaacucacacagacguccgcucgacuagcgggagcuaggagggucagccggaggaugcggagucuuuccgacuagcggagac

SEQ ID NO: 355 - RNA, 270 nt, Cocadviroid
  MK795553.1 Hop latent viroid strain CV_34, complete genome
ucuugagccccgagcuuaaccugcagaaguucacauaaaaacugccccucucgggaauacacuacgagacuaaccuguauggugccaagggcucgaagagggauccccggggaaacaaacucgagcgaggcggagaucgagccccaguucgugcgcggcgaccugaaguugcuucggcuacuucuugaucucguccugcguggaacggcuccuucuucacaccagccggaguuggaaacuacccggaggauacaacucuugagcgccgag

SEQ ID NO: 356 - RNA, 270 nt, Cocadviroid
  MK795538.1 Hop latent viroid strain CV_19, complete genome
ccgagcuuaaccugcagaaguucaaauaaaaagugacccuauggguaauacacuacgugacuaaccuguaugguggcaagggcuagaagagggauccccggggaaaccuacucgagcgaggcggagaucgagcgcuaguucgugcccggcgaccugaaguugcuucggcuacuucuuguucgcguccugaguggaacggcuccuucuucacaacagccggaguaggaaacuacccgguggauacaacucuugagcgccgagguuuugcug

SEQ ID NO: 357 - RNA, 270 nt, Cocadviroid
  DQ097185.1 Coconut cadang-cadang viroid isolate oil palm variant 270, complete genome
cuggggaaaucuacagggcaccccaaaaacuacugcaggagaggccgcuugagggauccccggggaaaccucaagcgaaucugggaagggagcguaccugggucgaucgugcgcguuggaggagacuccuucguagcuucgacgcccgcccccuucguagcuucgacgcccggccgccccuccucgaccgcuugggagacuacccgguggauacaacucaugcggcucuuaccuguuguuaguaaaaaaaggugucccuuuguagccccu

SEQ ID NO: 358 - RNA, 269 nt, Cocadviroid
  MK795612.1 Hop latent viroid strain CV_93, complete genome
cguuguguuuuccccagaggggcacuuuuuaugugaacuucugcagguuaagcucggcgcucaagaguuguauccaccggguaguuuccaacgccggcuggugugaagaaggagccguuccacgcaggacgcgaacaagaagaaaccgaagcaacuucaggucgccgcgcacgaacuggcgcucgaucuccgccucgcucgaguagguuuccccggggaucccucuucgagcccuugcaaccuuacagguuagucacguaguguuuucc

SEQ ID NO: 359 - RNA, 268 nt, Apscaviroid
  KY654684.1 Citrus viroid V isolate Ebi 1, partial genome
ucgacgaagggcggugagcaguaagccggacgguccccucgcggccgauccucuggagcucugcucuaagaucuucgcugcugaggcccgcgccgccgcucuuccgcgcugcuagucgagcggacguugguggucucccucucccugugcaauaaaauccagguggcgagugguguccccaggguaaaacacgauugguguucucccugggugaacaaccuugugguuccugugggucaccccgccccacggaauuauaagacaaggu

SEQ ID NO: 360 - RNA, 268 nt, Apscaviroid
  KY473927.1 Citrus dwarfing viroid isolate En-MT-Ver, complete genome
cuaucguuaugcguucauguucgucccaacgguaugcugcucgacaggaaccacgcgaaguuuccuccggggaaacaccaaucguguuuuacccuggaggcccaauccccuucuagccuagauaaaaacagauuaggauugggacaacucacucaguuguccgcucgacuagcgggagcuagaagggucugccggaggaugcggagucuuuccgacuagcggagacucuccgucuuuacuccacucggcgucaccacuuagcugccca

SEQ ID NO: 361 - RNA, 268 nt, Cocadviroid
  MK795596.1 Hop latent viroid strain CV_77, complete genome
caacucuuaagcgccgagcuuaaccugcagaaguucacauaaaaagugccccucuggggaauacacgacgggacuuaccuguaugguggcaacggcucgaagagggauccccggggaaaccuacucgagcgaggcggagagcgagcgccaguucgugcgcggcgcccugaaguugcuucggcugcuucuuguucgcguccugcguggaacggcuccuucuucacaccagccggaguuggaaacaaccggguggauacagcucuagagc

SEQ ID NO: 362 - RNA, 266 nt, Cocadviroid
  MK795620.1 Hop latent viroid strain CV_101, complete genome
aguuuccaacagcggcuggugugaagaaggcgccguugcacgcaggacgcgaacaagaagaagccgacgcaacuucaggucgccgcgcacgaacuggcgcucgagcuccgccucgcucgaguaggguuccccggggaucccucuccgagcccuugccaccauacagguaagucacguaguguauuccccagaggggcacuuuuuaugugaacuucugcagguuaagcucggcgcucaagaguugucugcaccggguaguuuccaac

SEQ ID NO: 363 - RNA, 265 nt, Cocadviroid
  MK795537.1 Hop latent viroid strain CV_18, complete genome
aucucucuugagcgccgagcuuaaccuccagaaguucacauaaaaagugccccucuggggaauacacuacaugacuuaccuguaugguggcaagggcucgaagagggauccccggggaaaccuacucgagcgaggcggagaucgagcgccaguucgugcgcggcgaccucaaguugcuucugcuacuucuuguucgcguccugcgaggaacggcuccuucuucacacaagccggagguggaaacuacccgguguauacaacuauu

SEQ ID NO: 364 - RNA, 264 nt, Cocadviroid
  MK795608.1 Hop latent viroid strain CV_89, complete genome
cacgcucuuccgaucuguaguuuccaacuccggcuggugugaagaaggagccguuccacgcaggacgcgaacaagaugaagccguagcaacuucaguuagccgcgcacgaacuggcgcucgaucuccgccuagcuagaguagguuuccccggggaucccuguucgagcccuugccuccauacagguaagucacguaguguauuccccagaggggcccuuuuuaugugaacuucggcaagaucggaagagcacaauucugaacuc

SEQ ID NO: 365 - RNA, 264 nt, Cocadviroid
  MF579864.1 Coconut cadang-cadang viroid isolate 323, complete genome
cuggggaaaucuacagggcaccccaaaaacuacugcaggagaggccgcuugagggauccccgggggaauucgccgcgccaaucgaauucccgcggccaaucugggaagggagcguaccugggucgaucgugcgcguuggaggagacuccuucguagcuucgacgcccggccgccccuccucgaccgcuugggagacuacccgguggauaacucacgcggcucuuaccuguuguuaguaaaaaaaggugucccuuuguagccccu

SEQ ID NO: 366 - RNA, 264 nt, Hostuviroid
  HE575348.1 Hop stunt viroid fragment, clone HSVd1, genomic RNA
accucucgguucgucccaaccugcuuuuugucuauuugagccucugccgcggauccucucuugagccccucuggggaauucucgaguugccgcaucaggcaagcaaaaaaaaacaaggcagggagguacuuaccugagaaaggagccccggggcaacucuucucagaauccagcgagaggcguggagagagggccgcggugcucuggaguagaggcucugccuucgaaacaccaucgaucgucccuucuucuuuaccuucuucu

SEQ ID NO: 367 - RNA, 263 nt, Cocadviroid
  MK795619.1 Hop latent viroid strain CV_100, complete genome
gcugcccgguggauacaacucuugagcgccgagcuuaaccugcagaaguucacauaaaaagugccccucuggggaauacacuacgugacuugccuguaugguggcaagggcucgaagagggauccccggggaagccuacucgagcgaggcggagaucgcgcgccaguucgugcgcggcgaccugaaguugcuucggcuucuucuuguucgcguccugcguggaccggcuccuucuucccccccgccggaguuggaaacucccc

SEQ ID NO: 368 - RNA, 263 nt, Cocadviroid
  MK795617.1 Hop latent viroid strain CV_98, complete genome
ugcagaaguucacauaaaaagugccccacuggggaauacacuacgggaguuaccuguauggugccaaggggucgaagagggauccccggggaaaccuacucgagcgaggcggagaucgagagacaguucgugcgcggcgaccugaaguugcuucggcuucuacuuguucucuuccugcguggaacggcuccuucuucacaccagccggaguuggaaacuacgcgguggauacaacucuugagcgccgagcuuaaccugcagaa

SEQ ID NO: 369 - RNA, 260 nt, Cocadviroid
  MK795626.1 Hop latent viroid strain CV_107, complete genome
ccggguaguuuccaacuccggcuggucugaagaaggagccgaaccacgcaggacccgaacaagaagaagccgaagcaacuucacgucgccgcgcacgaacuggcgcucgaucuccgccucgcucgaguagcuuuccccggggaucccucuucgcgcccuugccaccauacagguaagucacguaguguauuccccagaggggcacuuuuuaugugaacuucugcagguuaagcucggcgcucaagaguuguauccaccgg

SEQ ID NO: 370 - RNA, 259 nt, Cocadviroid
  MK795615.1 Hop latent viroid strain CV_96, complete genome
cauaaguucacauaaaaacugccccucuggggaauacacuacgugacuuaccuguaugguggcaagggcucgaagagggauccccggggaaacauacacgagcgaggcggagaucgagcgccaguucgugcgcggcgaccuguuguugcuucggcuucuucuuguucgcguccugcguggagcggcuccuucuucacaccggccggaguuggcaccucccggguggguagggcucuugagcggcgggcuugaccugcag

SEQ ID NO: 371 - RNA, 258 nt, Cocadviroid
  MK795591.1 Hop latent viroid strain CV_72, complete genome
cccgaggauaaaacucuugagcgccgcgcuuaaccugcagaaguucacauaaaaagugccccucuggggaauacacuacgugacuuaccuguaugguggcaagggcucgaagagggaaccccgggcaaaccuacucgagcgaggcggagaucgagcgccaguucgugcgcggcgaccugauguugguuaggcuuguucuuguucgcguccugcguugaacggcuccuucuucacaccagccgagaucggaagagcguc

SEQ ID NO: 372 - RNA, 258 nt, Cocadviroid
  MK795548.1 Hop latent viroid strain CV_29, complete genome
gugagcgccgagcuuaaccugcagcaguucacauaaauagugccccucuggggaauacacuacgugacuuaccuguaugguggcaauggaucgaagagggauccccggggaaaccuacucgagcgagucugagaucgagcgccaguucgugcgcggcgaccugaaguuucuucggauucuucuuguucgcguccugcguggaacggcuccuucuucacaccagccggaguuggaaacuacccggucgauacaacucuu

SEQ ID NO: 373 - RNA, 258 nt, Cocadviroid
  MK795535.1 Hop latent viroid strain CV_16, complete genome
agaucuaggggcacuuuuuaugugaacuucugcagguuaagcucggcgcucaagaguuguaucaaccguguaguuuccaacuccggcuggugugaagaaggagccguuccacgcaggacgcgaacaagaagaagccgaagcaaguugaggucgccgcgcacgaacuggcgcucgaucuccgccgcgcucgaguagguuuccccgaggaucccucuucgagcccuugccaccauagagguaagucacuuaguguauucc

SEQ ID NO: 374 - RNA, 257 nt, Cocadviroid
  MK795581.1 Hop latent viroid strain CV_62, complete genome
gguaagccucguaguggauuccccagaggggcacuuuuuaugugaacuucugcagguuaagcucggcgcucaagaguuguauccaccggguaguuuccaacucaggauggugugaagaaggagccguuccacgcaggacgcgaacaagaagaagcccaagcaacaucaggucgccgcgcacgaacuggcgcucgaucuccgccucgcucgaguagcaauccccgcggaucccacgucgagcccaagccagaaagcaa

SEQ ID NO: 375 - RNA, 257 nt, Cocadviroid
  MK795571.1 Hop latent viroid strain CV_52, complete genome
ucuuacuauauuacaacucuugagcgccgagauuaaccugcagaaguucacauaaaaagugccccucuggggaauacacuacgugacuuaccuguaugguggcaagggcucgaagaguuauccccggggaaaccuacucgagcgaggcggagaucgagcgccaguucgugcgcggcgaacugaaguuucuucggauucuucuuguucgcguccugcguggaucggcuccuucuucacaccagccggaguuggaaaaa

SEQ ID NO: 376 - RNA, 257 nt, Cocadviroid
  MK795561.1 Hop latent viroid strain CV_42, complete genome
acgcaugacgcuaacaagaagaagccgaagcaacuucagggcgccgcgcacgaacuggcgcucgaucuccgccucgcucgaguagguuuccccggggaucccucuucgagcccuugccaccauacagguaagucacguaguguauuccccagaggggcacuuuuuauguguacuucugcagguuaagcucggcgcucaagaguuguauccaccggguaguuucaaacuccggguggugugaagaauuagccuuuaca

SEQ ID NO: 377 - RNA, 256 nt, Cocadviroid
  MK795577.1 Hop latent viroid strain CV_58, complete genome
aaaaagugccccucuggggaauacacaacgagacuaaccuguaggguggcaagggcucgaagagggauccccggggaaaccuacucuaucgaggcggagaucgagcgccaguucgugcgcggcgaccugaaauugcuucggcuucuucuuguucgcguccugcguggaacggcuccuucuucacaccagccggagauggaaacuacccgguggauacaacucuugagcgccgagcuuaacagaucggaagagcguc

SEQ ID NO: 378 - RNA, 256 nt, Cocadviroid
  MK795527.1 Hop latent viroid strain CV_8, complete genome
cuccggcuggagugaagaaggagacguucaacgcaggacgcgaacaagaagaagccgaagcaacuucaggucgccgcgcacgaacuggcgcucgaucuccgccucgcucgaguagguuucccgggggaugccucuucgagcccuugccaccaaacagguaagucacguaguguauugcggagaggggcacuuuuuaugugaacuucugcagguuaagcucggcgcucaagagcuguauccaccguguagcuuccaa

SEQ ID NO: 379 - RNA, 256 nt, Cocadviroid
  KY654687.1 Citrus bark cracking viroid isolate Ebi 4, partial genome
cuggggaauuucucugcgggaccaaauaaaaacagcuuguggagggaacauaccugaggagggauccgggaggcgcgacggaucacuggcguccagcaccggaacaggagcucgaucuccuuccuuccaucgcuggcuccacauccgaucgucgcuucuuccuucgcgaccugagauagaaacuacccgguggauacaacucuuggguuguuccucccaggcuuguuaauaaaaauggcccgcguuugagaccccu

SEQ ID NO: 380 - RNA, 256 nt, Cocadviroid
  HH970095.1 Sequence 7 from Patent EP1664298
cuggggaauacacuacgugacuuaccuguaugauggcaaggguucgaagagggauccccggggaaaccuacucgagcgaggcggagaucgagcgccaguucgugcgcggcgaccugaaguugcuucggcuucuucuuguucgcguccugcguggaacggcuccuucuccacaccagccggaguuggaaacuacccgguggauacaacucuugagcgccgagcuuuaccugcagaaguucacauaaaaagugcccau

SEQ ID NO: 381 - RNA, 255 nt, Apscaviroid
  KY654686.1 Citrus dwarfing viroid isolate Ebi 3, partial genome
ggaaaacuccgugugguuccuguggggcacacccccuugccgaaaauaaaacgcagagagcuaaguuggugacgccgaguggaguaaagacggagagucuccgcuagucggaaagacuccgcauccuccggcagacccuccuugcucgccgcuagucgcgcggacuucaggagaguagccccaauccuaaucuguuuuuaucuaggcuagaaggggauugggccuccaggguaaaacacgauugguguuuucccc

SEQ ID NO: 382 - RNA, 255 nt, Cocadviroid
  MK795583.1 Hop latent viroid strain CV_64, complete genome
ucccuccacgacgcgcuuccgaucucgccggguaagcucggcgcucacgaguuguauacaccggguaguuuccaacuccggcuggugugaagaaggagccguuccacgcaggacacgaacaagaagaagccgaagcaacuucaggucgccgcgcacguacugucgcucgaucuccgccucgcucgaguagguuuccccggggaccgcucuucgagcccuugccuccauacauguacgucacguaguguauucccc

SEQ ID NO: 383 - RNA, 255 nt, Cocadviroid
  KT600318.1 Hop latent viroid isolate GVdC_HLVd01, complete genome
uugggauacaacucuugagcgccgagcuuuaccugcagaaguucacauaaaaagugccccucuggggaauacacuacgugacuuaccuguaugguggcaagggcucgaagagggauccccggggaaaccuacucgagcgaggcggagaucgagcgccaguucgugcgcggcgaccugaaguugcuucggcuucuucuuguucgcguccugcguggaacggcuccuucuucacaccagccggaguuggaaacuaaa

SEQ ID NO: 384 - RNA, 254 nt, Cocadviroid
  MK795593.1 Hop latent viroid strain CV_74, complete genome
cggggcacuuuuuaugugaacuucugcagguuaagcucggcgcucaagaguuguauccaccggguaguuuccaacuccggcuggugugaugacggagccguuccacgcaggacgcgaacaagaagaagccgaagcaacuucaggucgccgcgcacgaacuggcgcucgaucuacgccucgcuagaguagguuuccccugggaucccucuucgagcacuuuccaacauacagguaagucacguaguguauucacc

SEQ ID NO: 385 - RNA, 254 nt, Cocadviroid
  MK795589.1 Hop latent viroid strain CV_70, complete genome
aucuggguaguuuccaacuccggcuggugugaagaaggagccguuccacgcaggacgcgaacaagacgaagccgaagcaacuuccgggcgccgcgcacgaacuggcgcucgagcuccgccucgcucgagucgguuuccccggggaucccucuucgagcccuugcccccauacagguaagucacguaguguauuccccagaggggcacuuuuuaugugaacuucugcagguuaagcucggcgcucaagaucggag

SEQ ID NO: 386 - RNA, 254 nt, Cocadviroid
  NC_001471.1 Coconut tinangaja viroid, complete genome
cuggggaauucccacggcucggcaaaauaaaagcacaagagcgacugcuagagggauccccggggaaaccccuagcaaccgagguagggagcguaccuggugucgccgauucgugcugguugggcuucgucccuuccgagcuucgauccgacgcccggccgcuuccucgccgaagcugcuacggagacuacccgguggauacaacucuuugcagcgcccuguguaauaaaagcucgaguccgguuugcgccccu

SEQ ID NO: 387 - RNA, 253 nt, Cocadviroid
  MK795609.1 Hop latent viroid strain CV_90, complete genome
ccaccauacagguaagucacguaguguauuacccagacgggcacuuuuuaugucaacuucugcagguuaagcucggcgcucaugaguuguauccaggggguaguuuccaacuccggcuggugugaagaacgagccgcuccacgcaggacgcgaacaagaagaagccgaagcaacuucaggucgcugcgcacgaacuggcgcucgaucuccgcgucgcucgaguagguuuccgcggggaucccuguucgagccc

SEQ ID NO: 388 - RNA, 252 nt, Cocadviroid
  MK795605.1 Hop latent viroid strain CV_86, complete genome
aacuacccgguggauacaacucuuuagcgccuagcuuaaccugcagaaguucacauaaaaagugccacucuggggaauacacuacgugccuuaccuguaugguggcaagggcucgaagagggauccccggggaaaccuacucgagcgaggcggagaucgagcgccaguacgugcgcggcgaccugaaguugcuacggguucuacuuguucgcguccugcguggaacgucuccuucuucacaccagccggagu

SEQ ID NO: 389 - RNA, 252 nt, Cocadviroid
  MK795597.1 Hop latent viroid strain CV_78, complete genome
gucuuccgaucuugcagguuaagcucggcgcucaagaguuguauccaccggguaguuuccaacuccggcuggugugaagaaggagccguugcacgcaggucgcgaacaagaagaugccgaagcaaguucaggucgccgcgcacgaacuggcgcucgaucuccgccucgcucgaguagguuucuccggggaucccucuucgagaccuugccaccauacagguaauucacguaguguauuccccagaggggcac

SEQ ID NO: 390 - RNA, 252 nt, Cocadviroid
  MK795576.1 Hop latent viroid strain CV_57, complete genome
ucuuaagaucucuugaguuuaaucuaggcgcucaauauuuguauccaccggguaguuuccaacuccggcuugugugaagaacgagccguuccacgcaggacgagaacaagaagaagccgaagcaacuucagugcgccgcgcacgaacuggcgcucgaucuccgccucgcucgaguagguuuccccggggaucccucuucgaugccuugccuccauacagguaagucacguaguguauuccccagagcggccc

SEQ ID NO: 391 - RNA, 251 nt, Avsunviroid
  EU888595.1 Avocado sunblotch viroid isolate AvdMx.Hass8, complete genome
caagauuuuguaaaaaaacaaugaagauagaggaguaaaccuugcgagacucaucaguguucuucccaucuuucccugaagagacgaagugaucaagagaucgaagacgagugaacuaauuuuuuuuuaaauaaaaaguucaccacgacuccuccuucucucacaagucgaaacucagagucggaaagucggaacagaccugguuucgucaaacaaaguuuaaucauauccucacuucuuguucuaauaag

SEQ ID NO: 392 - RNA, 251 nt, Cocadviroid
  MK795630.1 Hop latent viroid strain CV_111, complete genome
gggcacuauuuaugugaacuacugcaggauaagcgaggcgcuaaagaguuguauccaccggguaguuuccaacuccggcuggugugaagaaggagccguuccacgcaggacgcgaacaagaagaauccgaagaaacuucaggucgccgcgcacgaacuggcgcucgaucuccgccucgcucgaguaggguuccccggggaucccgcuucgagcccuugccaccauacaggucagucacgaaguguauuccc

SEQ ID NO: 393 - RNA, 251 nt, Cocadviroid
  MK795628.1 Hop latent viroid strain CV_109, complete genome
ggcgcucaagaguuguauccaccggguagauuccaacuccggcaggugugaagaaggagccguuccacgcacgacgcgaacaagaagaagccgaagcaacuugaggucgccgcgcacgaacuggcgcucgaucuccgccucgcucgaguagguuuccccggggaucggucuucgagcccuugccaccauacagguaagucacguaguguauuccccagaggggcacccuuuaugugaacuucugcagguua

SEQ ID NO: 394 - RNA, 251 nt, Cocadviroid
  MK795616.1 Hop latent viroid strain CV_97, complete genome
cauaaguucacauaaaaacugccccucuggggaauacacuacgugacuuaccuguaugguggcaagggcucgaagagggauccccggggaaacauacacgagcgaggcggagaucgagcgccaguucgugcgcggcgaccuguuguugcuucggcuucuucuuguucgcgucgugcguggaacggcuccuucuucacugcagccggaguuggaaucuucccgguggauagaucucuugagcgccgagcuua

SEQ ID NO: 395 - RNA, 251 nt, Cocadviroid
  MK795549.1 Hop latent viroid strain CV_30, complete genome
gccggcuggugugaagaaggagccguuccacgcaggccgcgaacaagaagacgccgaagcaacuuaaggucgccgcgcacgaacuggcgcucgaucuccgccucgcucgaguagguuuccccggggaacccucuucgcgcccuugccaccauacagguaagucacguagaguauuccccagaggggcacuguuuaugugaacuucugcagguuaagcacggcgcucaagaguuguauccaccggguaguuu

SEQ ID NO: 396 - RNA, 251 nt, Cocadviroid
  MF579861.1 Coconut cadang-cadang viroid isolate 720, complete genome
cuggggaaaucuacagggcaccccaaaaacuacugcaggagaggccgcuugagggauccccggggaaaccucaagcgaaucugggaagggagcguaccugggucgaucgugcgcguuggaggagacuccuucguagcuucgacgcccggccgccccuccucgaccgcuugggagacuacccgguggauacaaucacacucacgcggcucuuaccuguuguuaguaaaaaaaggugucccuuuguagccccu

SEQ ID NO: 397 - RNA, 251 nt, Coleviroid
  X95291.1 Coleus blumei viroid 1-RG stem-loop RNA
uggcucgaacugacuagaacgguuuuaagaaaaacaccuggaguugcuuaccuggguucccuggcagcgcugcaacggaauccaugcccaccggagcgccaguucgugcggcgaccugaaguugcuucggcuucuucuuguucgugucguggaacggcucccguguacgcuggugacccgcgagcgauugcaaagguccugguucgcugacccggucucuuuucuaaacccuaguucagcuuauacgagcu

SEQ ID NO: 398 - RNA, 250 nt, Apscaviroid
  JX945672.1 Citrus dwarfing viroid isolate 6031, partial sequence
uugggagaguagccccaauccuaaucuguuuuuaucuaggcuagaaggggauugggccuccaggguaaaacacgauuggcguuuuccccggaaaacucccggugguuccuggggggcacacccccuugccgaaaauaaaacgcaaagggggaaaagggaacuuaccugucgucgucgacgaaggcagcuaaguuggugacggaccaaaaauucggcauucgucuucgcuugucacaaaauacccucgagu

SEQ ID NO: 399 - RNA, 250 nt, Apscaviroid
  JX945669.1 Citrus bent leaf viroid isolate 4039, partial sequence
uuucgucgacgucgaccaucagcuccucugccgcuuuauaacucuyyuyucuuucgcagguagggcugaggggugacaccacaggaaccacaagaagucucsgrggagcccucgagggguucgaggggcuycacccucuccccgcugcuacggaauucgugacaacmyaaagccgcuagcyagusaguruakcgucacmgasuugaagaguccgcgcgacuaacgaccagcauggcuggggugaacgaaa

SEQ ID NO: 400 - RNA, 250 nt, Cocadviroid
  MK795633.1 Hop latent viroid strain CV_114, complete genome
acccgguggauacaccucuugcgcgccgagcuuaaccugcagaaguucacauaaaaagugccccucuggggaauacacuacgugacuuaccuguaugguggcaagggcucgaagagggauccccggguaaaccuacucgagcgaggcggagaucgagcgccaguucgugcgcggcgcccugaaguugcuucggcugguuguuguucgcguccugcguggaccggaucauucuucacaccagagagcggaa

SEQ ID NO: 401 - RNA, 250 nt, Coleviroid
  MK477626.1 Coleus blumei viroid 1 isolate CbVd1_6B_13, complete sequence
uggcucgaacugacuagaacgguuuuaagaaaaaguucuuaccuggaguugcuuaccuggguucccuggcagcgcugcaacggaauccagugcccaccggagcgccaguucgugcgagaguugcuucggcuucuucuucguggaacggcuccgcguguacgccuggugacccgcgagcgauugcaaagguccugguucgcugacccgguucgaaccucuuuucuaaacccuaguucagcuuauacgagcu

SEQ ID NO: 402 - RNA, 249 nt, Avsunviroid
  KF562707.1 Avocado sunblotch viroid isolate Uruapan-3, complete genome
ugugagagaaggaggagucguggugaacuuuuauuaaaaaaauuaguucacucgucuucaaucucuugaucacuucgucucuucagggaaagaugggaagaacacugaugagucucgcaagguuuauuccucuaucuucauuguuuuuuuacaaaaucuuguuuauuagaacaagaagugaggauaugauuaaacuuuguuugacgaaaccaggucuguuccgacuuuccgacucugaguuucgacuua

SEQ ID NO: 403 - RNA, 247 nt, Cocadviroid
  MK795639.1 Hop latent viroid strain CV_120, complete genome
guaguguauuccccagaguggcacuuuuuaugugaacugcugcagguuaagcucggcgcucaagaguuguauccaccggguaguuuccaacuccggcuagugugaagaaggagccguuccacgcaggacgcgaacaagaagaaccccaagcaacuucaggucgccgcgcacgaacuggcgcucgaucaacgccucgcuccaguagguuucaccggggaucccugaucgagcacaucgguagagcaca

SEQ ID NO: 404 - RNA, 247 nt, Cocadviroid
  MK795614.1 Hop latent viroid strain CV_95, complete genome
ugagcgccuagcuuaaccugcagaaguucacauaaaaagugccccucuggggaauacacaacgugacuuaccuguaugguggcaaggacucgaagagggauccccggggaaaccuacucgagcgaggcggagaucgagcgccaguucgugcgcggcgaccuuaaguugcuucggcuucuucuuguucuagucgugcguggaacggcucguucuucacaccagccggaguuggauacuacccgguggu

SEQ ID NO: 405 - RNA, 246 nt, Cocadviroid
  MK795610.1 Hop latent viroid strain CV_91, complete genome
cccgguuagucacguaguguauuccggaguggggcacuuuuuaugugaacuucugcagguuaagcuaggcgcucaagaguuguauccacaggguaguuuccaacuccgacuggugugaagaaggagccguuccacgcaggacgcgaacaagaauaacccgaagcaacuucaggucgccgcgcacgaacuggcgcucgaucuccgcgucgcucgaguagguuuccgcggggaucccuguucgagccc

SEQ ID NO: 406 - RNA, 246 nt, Cocadviroid
  MK795567.1 Hop latent viroid strain CV_48, complete genome
gcaagggcucgaagagggaucccccgggaaaccuacucgagcgaggcggagaucgagcgccacuacgugcgcggcgaccugaaguugcuucggcuucuucuuguucgcuucaugcguggaacggcuccuucuucacaccagccggaguaugaaacuacccgguggauacaacucuugagcgccgagcuuaaccugcagaaguucacagaaaacgugccccucuggggagugcacuacgugacguac

SEQ ID NO: 407 - RNA, 246 nt, Cocadviroid
  MK795559.1 Hop latent viroid strain CV_40, complete genome
gcagguuaagcucggcgcucaagaguuguauccacaggguaguuucaaacaccggaugguuugaagaaggagccguuccacgcaggacgcgaacaauaagaagccgaagaaacuucaggucgccgcgcacgaacuggcgcucgaucuccgccgagcucgaguagguuuccccggggaucccucuucgagcccuagccaccauacagguaagacacguaguguauuccccagagcggcaccuauuau

SEQ ID NO: 408 - RNA, 246 nt, Cocadviroid
  MK795550.1 Hop latent viroid strain CV_31, complete genome
aaagggugccccucugggggguacacuacgugucuuuccuguaugguggcaggggcucgaagagggauccccggggaaaccuacucgagcgaggcggagauccagccccaguucgugcgcggcgaccugaaguugcuucggcuucuucuuguugggguccugcguggaacggcuccuucuucacaccagccggaguuggaaacuacgcgguggauacaacucuugagcgccgagcuuaaccugcag

SEQ ID NO: 409 - RNA, 244 nt, Cocadviroid
  MK795592.1 Hop latent viroid strain CV_73, complete genome
uagacgcauauaguccgagcuggggcacuuuuuaugugaacuucugcagguuaugcucggcgcucaagaguuguaucgaccggguaguuuccaacuccggcuuguguuaagaaggagccguuccacgcaggacgcgaacaagaagaacacgaagcaacuucaggucgccgcgcacgaacuggcgcucgaucuccgccucgcucgaguaaguuuccccggguaucccucuucgagcccuugacac

SEQ ID NO: 410 - RNA, 243 nt, Cocadviroid
  MK795632.1 Hop latent viroid strain CV_113, complete genome
ggcgaaaacacuacgugacguaccugaaugguggcaaggccucgaagagggaucaccggggaaaccuacucgagcgaggcgcagaucgcgcgccaguucgugcgcggcgaccugaaguugcuucggcuuguuguuguucgcguccugcguggaacggcuccuucuucacaccagccggaguuggaaacuacccgguggauacaacucuagagccccgaacagaacggaauagcgucaugaagg

SEQ ID NO: 411 - RNA, 243 nt, Cocadviroid
  MK795606.1 Hop latent viroid strain CV_87, complete genome
ggugaagaaggagccgcuccacgaggacgcgaacaagaacaacccgaagcaacuucaggccgccgcgcacgaacuggcgcucgaucuccgccucgcucgagcaggcuuccccggggaucccucuucgagcccuugccacccuacaggcaagucacguaguguauuccccagaggggcacuuuuuaugugaacuuccgcagguuaagcucggcgcucaagaguuguauccaccggguaguuucc

SEQ ID NO: 412 - RNA, 243 nt, Cocadviroid
  DL463096.1 Detection method of Hop latent virus, primer set and kit for detection of Hop latent virus
aguuccuugcucguagaagggccgcuauccgcgugacuaaugcugggcuagaaacaggccggccagcgcucaagcccacaccugacaugcugccugauccuaccaacccauauaacaaacccucauuagaugcuuuguugaugauaaagccaagaguugugucuaacaauauggcuaccucugaggauaugaugaagaucugcguugaucuugagggguugggcgugccuacugagcacgugc

SEQ ID NO: 413 - RNA, 242 nt, Cocadviroid
  MK795569.1 Hop latent viroid strain CV_50, complete genome
uggugugaagaaggagccguuccacgcaggucgcgaacaagaugaaggcguagcaaguucaggucgccgcgcacgaacuggcgcucgaucuccgccucgcucgaguagguuuccccggggaucccuguucgcgcccuugccaccauacagguaagucacguaguguauuccccacaggggcacuuuuuaugugaacuucugcagguuaagcucggggcucaagaguaguaugcacaggguag

SEQ ID NO: 414 - RNA, 241 nt, Cocadviroid
  MK795573.1 Hop latent viroid strain CV_54, complete genome
cagccgaauacacuaccggacuuaccugaaugguggcaagggcucgaagagggauccccggggaaaacuacucgagcgaggcggagaucgagcgccagaucgagcgcggcgaccugaaguugcuucggcuucuucucuuucgcguccugcguggaacggcuccuucuucacaccagccggaguuggaaacuacccgguggauacaagucuugagcgccgagcuuaacgugcauaaguucac

SEQ ID NO: 415 - RNA, 240 nt, Cocadviroid
  MK795566.1 Hop latent viroid strain CV_47, complete genome
uacgugacuuaccuguaugggggcaagggcucgaagagggauccccggggaaaccuacucgaacgaggcggagaucgagcgccaguucgugcgcggcgaccugaaguugcuucggcuucuucuuguuagcguccuucguggaacggcuccuucuucacaccagccggaguuggauaguacccgguggauacuacucuugagcgccgagcuuaaccugcagaaguuaacauaaaaagugcc

SEQ ID NO: 416 - RNA, 239 nt, Cocadviroid
  MK791746.1 Hop latent viroid isolate CR3A, partial genome
  misc_feature (9)..(9): n is a, c, g, or u
  misc_feature (237)..(237): n is a, c, g, or u
cgagcgccnguucgugcgcggcgaccugaaguugcuucggcuucuucuuguucgcguccugcguggaacggcuccuucuucacaccagccggaguuggaaacuacccgguggauacaacucuugagcgccgagcuuaaccugcagaaguucacauaaaaagugccccucuggggaauacacuacgugacuuaccuguaugguggcaagggcucgaagagggauccccggggaaaccnag

SEQ ID NO: 417 - RNA, 238 nt, Cocadviroid
  MK795556.1 Hop latent viroid strain CV_37, complete genome
gcgcgccagaguuguauccaccggguaguuuccaacuccugcuggugugaagaaggagccguuccacgcaggacgcgaacaagaagacgccgaagcaacuucaggucgccgcucacgaacuggcgcgcgaucuccgcgucgcucgaguagguuuccccggggaucccucuucgagcccuugccaccauacagguaaguaacguaguguauuccccagcggggcacuuuuuaugugaac

SEQ ID NO: 418 - RNA, 237 nt, Cocadviroid
  MK795623.1 Hop latent viroid strain CV_104, complete genome
acgcgaacaagaagaagccguagcuacuucagggcgcggcgcacgaacuggcgcucgauauccgccucgcucgaguagguuuccccggggaucccucuuagagcacuugccaccauacagguaaguaacguaguguauuccccagaggggcacuuuuuauuugaacuucugcagguuaagcucggcgcucaagaguuguauccaccggguaguuuccaacuacggcuggugugaaga

SEQ ID NO: 419 - RNA, 237 nt, Cocadviroid
  KT964100.1 Coconut cadang-cadang viroid isolate RP1, partial genome
acgucaagcgaaucugggaagggagcguaccacccugcaucgugcgcguuggaggagacuccuucguagcuuagacgcccggccgccccuccucgaccgcuugggagacuacccgguggauacaacucacgcggcucuuaccuguuguuaguaaaaaaaggugucccuuuguagccccucuggggaaaucuacagggcaccccaaaaacuacugcaggagaggccgcuugagggauc

SEQ ID NO: 420 - RNA, 235 nt, Apscaviroid
  MK888699.1 Citrus dwarfing viroid isolate SL-06, complete genome
cuccgcuagucggaagacuccgcauccuccggcugacccgcucaguuugccgcuagucgagcggacuucagggaguagccccaauccuaaucuguuuuuaucuaggcuagaaggggauugggccuccaggguaaaacacgauugguguuuccccggaggaaacuccgugugguuccuguggggcacacccccuugccgaaaauaaaacgcagagagggaaaaggaaacuuaccug

SEQ ID NO: 421 - RNA, 233 nt, Cocadviroid
  MK795574.1 Hop latent viroid strain CV_55, complete genome
cuuccgcgcugcuggugggcagaaggagccguuccacgcaggacgagaacaagaagaagccgaagcaacuuccggucgccgcgcacgaacuggcgcucgaucuccgccucgcucgaguagguuuccccggggcucccgcuucgagcccuugccaccauacagguaagucacguaguguauuccccagaggggcacuuuuuaugugaacuucugcagguuaagcucggcccgcc

SEQ ID NO: 422 - RNA, 233 nt, Cocadviroid
  MK795557.1 Hop latent viroid strain CV_38, complete genome
agacgugugcgcguccgaucugcucgaagagggauccccggggaaaccuacucgcgcgaggcggagaucgagcgccaguucgugcgcggcgaccugaagucgcuucggcuucuucuuguucgcguccugcguggaacggcuccuucugcgcaccagccggaguuggaaacuacccgguggauacaacucuugaucgacgagcuuaaccugcagaaguucacauauaaagugcc

SEQ ID NO: 423 - RNA, 233 nt, Cocadviroid
  MK795554.1 Hop latent viroid strain CV_35, complete genome
cucgaagagggauccccggggaaaccuacucgagcgaggcggagaucgagcgacaguucgugcgcggcgaccugaaguugcuucggcuucuucuuguucgcguccugaguggaacugcuccuucuucacaccagccggaguaggaaacuacccggaggauacaacucuugagcgccgagcuuaaccugcacaacaucacauaaaaagucccccucuggggaauacacuacgug

SEQ ID NO: 424 - RNA, 232 nt, Cocadviroid
  MK795599.1 Hop latent viroid strain CV_80, complete genome
ccuacacgaggcucuuccgaucucugcagguuaagcucggcgcucaagaguuguauccaccggguaguuuccaacuccggcuggugugaugauggagccguuccacgcaggacgcgaacaagaagaagccgaugcaacuucaggucgccgcgcacgaucuggcgcucgaucuccgccuggcucgaguagguuuccccggggaucccucuucgagcacuugccaccauacagu

SEQ ID NO: 425 - RNA, 232 nt, Cocadviroid
  MK795560.1 Hop latent viroid strain CV_41, complete genome
cauaaaaacugcccgucuggggaauacacuaccugacuuaccuguaugguggcaaggguucgaagagggauccccggggaaacguagucgagcgaggcggagaucgagcgccaguucgugcgcggcgaccagaagaugcuucggcuucuucuuguucgcggccugcguggaacggcuccuucuucacaccagccggaguuggaaacuacccgguugauacaacucuugagcg

SEQ ID NO: 426 - RNA, 231 nt, Apscaviroid
  MG879257.1 Citrus dwarfing viroid isolate E20-Cu-Ver, complete genome
aaacagauuaggauugggacaacucacucaguuguccgcucgacuagcgggagccauacgagauuccacuguaccugucgucgucgacgaaccucucgcccacccuucugcucccgcacucgcgcacaacugaauacccuugucccacuccuauccuguuuuacucuaugcuaugacuugauaggugcuccuuggugggaacuaguguuuaccccgaugaaacucccuuga

SEQ ID NO: 427 - RNA, 230 nt, Apscaviroid
  JQ080280.1 Citrus bent leaf viroid isolate CBLVd-2, complete genome
cggagacuucuugugguuccuguggugacaccccucagcccuaccugcgaaagaaaaaaagaguuagaaggcggcagaggagcugacuggucgucgucgauccccuucacccgagcgcugcuugccgcuagucgagcggacuuccaagucucccucccgcgccgcuuuucuuuucuccgauucuguagcagcggggagagggugaagccccugaaccccugagggcuccu

SEQ ID NO: 428 - RNA, 230 nt, Cocadviroid
  MK795590.1 Hop latent viroid strain CV_71, complete genome
aaaaagugccccucuggggaauacacuacgugacguuccuggaggguggcgggggcucgaagagggcuccccggggaagccuacucgagcgaggcggagaucgagcgccuguucgugcgcggcggccugaaguugcuucggcuucuucuuguucgcguccugcguggaacggcuccuucuucacaccagccggaguuggaaacuacccgguggauacaacucuugaucgc

SEQ ID NO: 429 - RNA, 228 nt, Apscaviroid
  MG879255.1 Citrus dwarfing viroid isolate E14-SR-Ver, complete genome
ccugucauucgcgucacaguuguagcgugccagaacguuacccugcgcgaguuuccuccggggaaacaccaaucguguuuuacccuggaggcccaauccccuucuagccuaaauaaaaacagauuaggauugggacaacucacucaguuguccgcucgacuagcgggagcuagaagggucgacgagaauacggacgacgaccaaagagacgagacgcaccgccuuuaa

SEQ ID NO: 430 - RNA, 228 nt, Cocadviroid
  MK795641.1 Hop latent viroid strain CV_122, complete genome
aaagugacccucuguggaauacacuacgugacuuacauguaugguggcaagggcucgaagagguauccccggggaaaccuacucgagcgaggcggagaucgagcgacaguucgugagcggcgaccugaaguugcuucggcuucuucuuguucgcguccugcuuguaacggcuccuucuucacaccagccggaguuggaaacuacccgguugauacaacucuugagagg

SEQ ID NO: 431 - RNA, 227 nt, Cocadviroid
  MK795611.1 Hop latent viroid strain CV_92, complete genome
caagaguuguauccaccggguaguuuccaaguccggguggugugaugaaggagccguuccacgcaggacgcgaacaagaagaagccguagcaacuucaggucgccgcgcacuaacuggcgcuugaucuccgccucgcucgaguagguuuccccggggaucccgcuucgugcccuugccaccauacagguaagucacguaguguauucgccccaggggcacuuuuuau

SEQ ID NO: 432 - RNA, 227 nt, Cocadviroid
  MK795604.1 Hop latent viroid strain CV_85, complete genome
cccagaggggcacuuuuuaugugaacuucugcagguuaagcucggcgcucaagaguuguauccaccggguaguuuccaacuccgucugguuugaagaaggagccguuccacgcaggacgcgaacaagaagaagccgacgcaacgucagggcgccgcgcacgaacgggcgcucgcgcgccgccucgcucgaguagguuuccccggggaucccucuucgagcccuugcg

SEQ ID NO: 433 - RNA, 226 nt, Cocadviroid
  MK795644.1 Hop latent viroid strain CV_125, complete genome
ccaguggggcacuuuuuauguguucuucugcugguuaaucucggcgcucaagaguuguauccaccggguaguuuccaacuccagcuggugugaagaaggagccguuccacgcaggacgcguacuagaagaagccgaagcaacuucaggucgccgcgcacgaacuggcgcucgaucuccgccucgcucgaguagguuuccccggggaucccucuucgagcccucgcc

SEQ ID NO: 434 - RNA, 225 nt, Avsunviroid
  AF404073.1 Avocado sunblotch viroid isolate CF59, complete genome
caagauuuuguaaaaaaacaaugaagauagaggaguaaaccuugcgagacucaucaguguucuucccaucuuucccugaagagacgaagugaucaagagauugaagacgagugaacuaacgacuccuccuucucucacaagucgaaacucagagucggaaagucggaacagaccugguuucgucaaacaaaguuuaaucauauccucacuucuuguucuauuuaa

SEQ ID NO: 435 - RNA, 224 nt, Cocadviroid
  MK795531.1 Hop latent viroid strain CV_12, complete genome
cuuaccuguauaguggcaagggcucgaagagguauccccggggaaaccuacucgagcgaggcggagaucgagcgccagggcgugcgcggcgaccugaaguugcuucggcuucuucuuguucgcggcgugcguggaacggcuccuucuucacaccagccggaguuggaaacuacccgguggauacaacucuugagcgccgagcuuaaccugcuagaucggaggag

SEQ ID NO: 436 - RNA, 220 nt, Apscaviroid
  JX892934.1 Grapevine yellow speckle viroid 1 clone 33 symptomatic, complete genome
ccgcuagucgagcggacuuggucucuucugcccaaagcccuuuuucuuucaacugagcuuguuccaacgcgccccgcgaguggaauccccggaaccccugcaaagagguccucggaucacuuuccugugguuccugugguuacaccucggaaggccgccgcggaccugcaaagaagaagauaggggcagagggggagugagccucgucgucgacgaaggg

SEQ ID NO: 437 - RNA, 220 nt, Cocadviroid
  MK795602.1 Hop latent viroid strain CV_83, complete genome
cuaucuagcagguuaagcucggcgcucaagaguuguauccaccggguaguuuccaacuccggcuggggugaagaaggagccguuccacgcaggacgcgcacacgaagaagccgaagcaucuucaggucgccgcgcacgaacuggcgcucgaucuccgccucgcucgaguauguuuccccggggagcacucuucuagcccuugccaacauaccuguaauuc

SEQ ID NO: 438 - RNA, 220 nt, Cocadviroid
  MK795578.1 Hop latent viroid strain CV_59, complete genome
aaaaagugccccacuggggaauacacuaccuuacuaaccuguaucguggcaagggcucgaagagggauccccggggaaaccuacucgagcgaggcggagaucgagcgcccggucgugcgcggcgaccugaaguugcuucggcuucaucuuguucgcguccugcguggaacagcuccuucuucacaccagccggagauggaaacuacccgguggauacaac

SEQ ID NO: 439 - RNA, 219 nt, Cocadviroid
  MK795621.1 Hop latent viroid strain CV_102, complete genome
auccaccguguaguuuucaacuccggcuguugugaagaaggagccguuccucgcaggacgcgaacaagaagaagccgaagcaacuucaggucgccucgcacgaacugucgcuagaucuccgccucgcucgaguagguuuccccggggaucccucuucgagcccuugccaccauacauuuaagucacguaguguauuccccagaggggcacuuuuuaugu

SEQ ID NO: 440 - RNA, 218 nt, Cocadviroid
  MK795552.1 Hop latent viroid strain CV_33, complete genome
ugcggguuaagcucggcgcucaagaguuguauccaccggguaguuuccaacuccugcuggugugaagaaggagccgguccacgcaggacgcgaacaagcagaagccgaagcaacuacaggucgccgcgcacgaacucgcgcucgaucugcgccucgcucgauuagguuucccgggggaucccucuucgagcccuugccaccauacagguaagucacgu

SEQ ID NO: 441 - RNA, 217 nt, Apscaviroid
  LC427232.1 Citrus viroid VI persimmon, genomic RNA, partial sequence
ugccgcuagucgagcggaccacagagugacucuccugcccuauuuucuacgagagccggcgguggaguccucaggguaaaacacgauugguguuuccccggaggaaacuccgugugguuccugugggacaccccccggcacccucaaugaaaagaaaggcaaggaaacucaccugucgucgucgacgaaggcaugugagcugcuacucgaugaagag

SEQ ID NO: 442 - RNA, 217 nt, Apscaviroid
  JX892928.1 Grapevine yellow speckle viroid 1 clone 15 nonsymptomatic, complete genome
ccucggaucacuuuccugugguuccugugguuacaccucggaaggccgccgcggaccugcaaagaagaagauaggggcagagggggagugagccucgucgucgacgaaggggcuagucgagcggacuuggucucuuccgcccaaagcccuuuuucuuacuacugagcuuguuccaacgcgccccgcgaguggaauccccggaaccccugcaaagagg

SEQ ID NO: 443 - RNA, 217 nt, Cocadviroid
  MK795598.1 Hop latent viroid strain CV_79, complete genome
gugaacuucugcagguuaagcuccgcgcucaagaguuguauccaccggguaguucccaacuccggcuggugugaagaaggagccguuccacgcaggacgccaacaagaagaagccgaaucaacuucagguggccgcgcacgaaguggcgcucgaucugcgccucgcucgaguagguuuccccggggauccguguucgagcccuugucaccauacagg

SEQ ID NO: 444 - RNA, 217 nt, Cocadviroid
  MK795546.1 Hop latent viroid strain CV_27, complete genome
gcaagggcucgaagagcgauccccggcgaaaccaacucgagcgaggcggagaucgcgcgccaguucgugcgcggcgaccugaaguugcuucggcuucugcuuggucgcguccugcguggaacggcuccuucuucacaccagccggaguuggaaacuacccgguggagacaacucuugagcgccgcgcuuaaccugcagaaguucacagaaaaagugc

SEQ ID NO: 445 - RNA, 217 nt, Pelamoviroid
  JQ904596.1 Chrysanthemum chlorotic mottle viroid, partial sequence
caguuucggcuugugcgggaguaaagcuguugcucucuccacagccucguccggguccccaccccuuuccuccuggggugggacggagguauuuaccuuuacuccgcuuagaggggaagguaacuaacuucccucaaaaaaaaaaaggggguuuccuuaauaaaguggggaaaaaaauuuuuuuuuacuuccccucucguuggauauucuccucgga

SEQ ID NO: 446 - RNA, 216 nt, Cocadviroid
  MK795532.1 Hop latent viroid strain CV_13, complete genome
ggcgcucaugaguuguaucccccggguaguuuccaacuccggcuggugugaagaaggagccguuccacgcaggacgcgaacaagacgaagccgaagcaacuucaggacgccgcgcacgaacuggcgcccgaucuccgccccgcacgaguagguuuccccggggagcccucuucgagcccuugccaccauacagguaagucacguaguguauucccc

SEQ ID NO: 447 - RNA, 215 nt, Pospiviroid
  JX945671.1 Citrus exocortis viroid isolate E14, partial sequence
cccggaaggauuuuuucccccgcgccuuuuuuuuucuuuuccugccugcagggucaggugagcaccacaggaaccucaagaaagaucccgagggacccaaucuaggguuccgagggcuuucacccucgccuugagagcagugaagacaaagaaaagcgguuugggguugaagguucaguuguuucccccgggugguagccagagagaagcuccgg

SEQ ID NO: 448 - RNA, 214 nt, Cocadviroid
  MK795582.1 Hop latent viroid strain CV_63, complete genome
guaugguggcacgggcucgaagagggauccccggggaaaccuacgcgagcgaggcggagaucgagcgccaguucgagcgcggcuaccugaaguugcuucggcuucuucuuguucgcuuccugcuuggaacggcuccuucuucacaccagccggaguuggaaacuacccgguggauacaacucuuuagcgccgagcuuaaccugcagaaguucac

SEQ ID NO: 449 - RNA, 213 nt, Apscaviroid
  MG879254.1 Citrus dwarfing viroid isolate E12-SR-Ver, complete genome
ccacacggaguuuccuccggggaaacaccaaucguguuuuacccuggaggcccaauccccuucccuaaaaaacagauuaggauugggacaacucacucaguuguccgcucgacuagcgggagcaagggucugccggaggaugcggagccacgacaacuacaggagaaucuccgucacgacuccacucggcgucaccaacuuagcugccaauag

SEQ ID NO: 450 - RNA, 212 nt, Apscaviroid
  HE601745.1 Apple scar skin viroid partial genome, isolate Rohru
agacccuucgucgacgacaacaggugaguuccuucuucuccucgauuucuuuaucuaucugcguuggcggggcgaaccacaggaaccgcacgguguuuaccgggaaucaccuauuguguuuuacccugggaacccacccgccgcugcgucaaagaaaaagagcgugggagaacagggggcuccacccggaguccgcucgacuagcgggacaa

SEQ ID NO: 451 - RNA, 211 nt, Pospiviroid
  KP454039.1 Potato spindle tuber viroid isolate CVN259, partial sequence
caguucauccaccgggucccuuaugcuucaguuguuucccccggguaguagccgaagcgacagcgcaagggggcgaggggugauccugcgggcgcgaggaaggaaaccugaagaaaggaagggugaaaacccuguuucagcaggaauuacuccugucggccgcgaggcacuccccaccgcgccuuuugccaguucgcuccagguuuccccg

SEQ ID NO: 452 - RNA, 208 nt, Cocadviroid
  MK795524.1 Hop latent viroid strain CV_5, complete genome
ggcaagggcucgaagagggauccccggggaaaccuacucgagcgaggcggagaucgagcgccaguucgugcgcggcgaccugaaguugcuucggcuucuucuuguucgcguggugcguggaacggcuccuucuucacucgagccggaguuggauacuucccgguggauacuacucuugagcgcggagcuuaaccugcagaaguucaca

SEQ ID NO: 453 - RNA, 206 nt, Cocadviroid
  KC121568.1 Citrus bark cracking viroid isolate 6035, partial sequence
ggguggauacaacucuuggguuguuccucccaggcuuguuaauaaaaauggcccgcguuugagaccccucuggggaauuucucugcgggaccaaauaaaaacagcuuguggagggaacauaccugaagagggauccccggggaaaucucuucagacucgucgaggggagggcgccgcggaucacuggcguccagcaccggaacagg

SEQ ID NO: 454 - RNA, 205 nt, Cocadviroid
  MK795575.1 Hop latent viroid strain CV_56, complete genome
cugcaguuuaagcucggcgcucaauaguuguauccacaggguaguuuccaacuccggcuggugugaagaaggcgccguuccacgcaggacgcgaacaagaagaagccgaagcaacuucagggcgccgcgcacgaacuggcgcgcgaucuccgccucgcucgagucgguguccccggggaucccucuucgagcccuugccaccaua

SEQ ID NO: 455 - RNA, 205 nt, Cocadviroid
  MK795521.1 Hop latent viroid strain CV_2, complete genome
ggcaagggcucgaagagggauccccggggaaaccuacucuagcgaugcggagaucgagcgccaguucgugcgcggcgaccugaaguugauucugcuucuucuuguucgcguccugcguggaacggcuccuucuucacaccagccggaguuggaaacuacccgggggauucaacucuugagcgcccagcuuaaacugcagaaguuc

SEQ ID NO: 456 - RNA, 204 nt, Apscaviroid
  MG879258.1 Citrus dwarfing viroid isolate E21-AR-Ver, complete genome
cgucgcacgaaacuccggcagacccuuuucuugccgcuagucgagcggaccacugaauguauccuuacucuuaaucuguuuuuuucuuugcgagguuuccccucaggugacucccuguggguacccuggggaacuccccccuguggaaaauauaacacgcagaggggcagggucuuuuuuuguuguccucaccaaaaaaacuga

SEQ ID NO: 457 - RNA, 201 nt, Apscaviroid
  MG879256.1 Citrus dwarfing viroid isolate E19-Cu-Ver, complete genome
gcuaaacagauuaggauugggacaacucacucaguuguccgcucgacuagcgggagccauagcgagaugccacugcuaccugucguuguagcggugcuucucgucgacgauucuggcuccugcucuagcggacaacugaauacccuugucccacuccucuccuguuuuacuuugugcuaugacuugauaggugcuuauuag

SEQ ID NO: 458 - RNA, 198 nt, Pospiviroid
  MK330991.1 Potato spindle tuber viroid isolate 21819070-B sequence
gggauccccggggaaaccuggagcgaacuggcaaaaaggcgcgguggggagugcccagcggccgacaggaguaauuccugcagaaacaggguuuucacccuuccuuucuucggguuuccuuccucgcgcccgcagguccaccccucgcccccuuugcgcugucgcuucggcaacuacccgguggawacaacugaagcu

SEQ ID NO: 459 - RNA, 197 nt, Pospiviroid
  DQ846884.1 Citrus exocortis viroid from Verbena x hybrida, partial genome
ccggggaaaccuggaggaagucgaggucgggggggaacagcugguucggucgccgcggaucacuggcguccagcgaagaaacaggaguguggcuuccuuccuuucgcugcuggcucccauccgaucgucguugaagcgccacgcccccucgcccggagcuucucucuggagacuacccgguggaaacaacuaaagcu

SEQ ID NO: 460 - RNA, 180 nt, Avsunviroid
  HC918585.1 Sequence 8 from Patent WO2010061186
gcucgagacuugugagagaaggaggagucguggugaacuuuuauuaaaaaaauuaguucacucgucuucaaucucuugaucacuucgucucuucagggaaagaugggaagaacacugaugagucucgcaagguuuacuccucuaucuucauuguuuuuuuacaaaaucuugggcgcgccc

SEQ ID NO: 461 - RNA, 178 nt, Cocadviroid
  MF616006.1 Coconut cadang-cadang viroid isolate UPM 3-3 clone 2, partial genome
cuggggaaaucuacagggcaccccaaaaacuacugcaggagaggccgcuugaggguuccccggggaauccucaagggaaucugggaagggaggguaccuuggccagcucacguucguaggaugagacuccaucguaccuucgacgcugagaugccccuccacgauugcucgggagacu

SEQ ID NO: 462 - RNA, 178 nt, Cocadviroid
  MF616002.1 Coconut cadang-cadang viroid isolate UPM 1-2 clone 2, partial genome
cuggggaaaucuacagggcaccccaaaaacuacugcaggagaggccgcuugagggauccccgggggaauucgccgcgccaaucgaauucccgcggccaaucugggaagggagcguaccuggguagaucgugcgcguuggaggagaagccaccgucgcugagacgaacgaccgccccuc

SEQ ID NO: 463 - RNA, 175 nt, Cocadviroid
  MF616004.1 Coconut cadang-cadang viroid isolate UPM 2-2 clone 2, partial genome
cuggggaaaucuacagggcaccccaaauauacuacgugcaggagaggccgcuugagggauccccggggaaaccucaagcgaaucugggaagggagcguaccuggguagaucgugcugcguuggaggagaguccaucgucgcugagaugccccgcagccccuacuagacagcauug

SEQ ID NO: 464 - RNA, 144 nt, Pelamoviroid
  FJ647358.1 Chrysanthemum chlorotic mottle viroid isolate plant 3 clone hammerhead 04, partial sequence
aagagguccggcaccugaugucgguguccugaugaagauccaugacaggaucgaaaccucuuccaguuucggcuugugcgggaguaaagcuuucgcucucuccacagccucaucaggaaacccacuucaggucucgacuggaag

SEQ ID NO: 465 - RNA, 143 nt, Hostuviroid
  EU925587.1 Hop stunt viroid isolate 1, partial sequence
ccaaccuguuuuuugucuaucugauccucugccgcggauccucucuugacccccucuggggaauucucgaguugccgcaucaggcaugcaaugaaaaaacuuggcagggagguacuuaccugaaaaaggagccccggggcaac

SEQ ID NO: 466 - RNA, 97 nt, Cocadviroid
  MF616001.1 Coconut cadang-cadang viroid isolate UPM 1-2 clone 1, partial genome
cgaucgugcccguuggaggagacuccuucguagcuucgacgcccggccgccccuccucgaccgcuugguuuuuagagagcuaccgcgguggauacaa

SEQ ID NO: 467 - RNA, 65 nt, Pospiviroid
  S52178.1 [potato spindle tuber viroid, Other Genetic Material RNA, 65 nt]
cuguccucauuaagggcggcuuugucccaaaagugggaaggaaagaagcccacaggaaggagcgc

SEQ ID NO: 468 - RNA, 452 nt, Artificial Sequence
  Synthetic Construct
cgggaacuuucuugagguuccuguggugcucaccugacccugcaggcagaaaagaaaaaagaggcggcgggggaagaaguccuucagggauccccggggaaaccuggaggaagucgaggucgggggggacagcugcuucggucgccgcggaucacuggcgccagcggagaaacaggarcucggucgccgcggaucacuggcguycagcggagaaacaggagcucgucuccuuccuuucgcugcuggcuccacauccgrucgucgcugagcuccuuccuuucgcugcuggcuccacauccgaucgucgcugaagcgccygcccccucgcccggagcuucucucuggagacuacccgguggaaacaacugaagcuucaaccccaaaccgcuuuucuuauaucuuycugcucuccgggcgagggugaaagcccuuggaacccuagaugggucccu

SEQ ID NO: 469 - RNA, 456 nt, Artificial Sequence; identical to SEQ ID NO: 53
  Synthetic Construct

SEQ ID NO: 470 - RNA, 448 nt, Artificial Sequence; identical to SEQ ID NO: 54
  Synthetic Construct

SEQ ID NO: 471 - RNA, 371 nt, Artificial Sequence
  Synthetic Construct
uuagccuuccugaugaguccguuccaargacgaaaccgucucuccugacucccgguucugguuagguyugguaycccuaccaguuacuuccgguaacuaggaguuuguagugacguaaguuucguycuaaragracucauyagguagccuaauagacuacgacuuacgcggurggucgugaayaagcccggaguacauucccaauggaacuagggaaccccuccggucuuruccaaccucuguuucggcagaggayaccgugcuccayggyugaaggccrugggcccgagagaagucgcucucucucgcccuyacauccucucaggcauagagaugaccauuugccuagggugagucuuuaaguagacaca

SEQ ID NO: 472 - RNA, 434 nt, Artificial Sequence; identical to SEQ ID NO: 56
  Synthetic Construct

SEQ ID NO: 473 - RNA, 391 nt, Artificial Sequence
  Synthetic Construct
uccguuccaaggagaaaccgucuyccagacuccgguucugguuggguuaguauaccaacuaguuacuuccgguaacuuggrguuuguygacruaaguuucguccuaaragrrcucaucagguagccuaauagacuacgacuuaygcgguaggucgugaayaagccggaguacauuyccaugrayaggraaccccuccggucuuruccaaccucuguuucggcagaggauaccgurcucccagcuguaaggcuggggcccgagagaagucgcucucucucgcccuucruccucucaggcauagagaugaccauuugccuagggugagucuuuaaguagacacagccuaucuauggygaygaggggcacggguuuurcacccccaugacgguc

SEQ ID NO: 474 - RNA, 433 nt, Artificial Sequence
  Synthetic Construct
graggcuauuagccuyccugaugaguccguuccaaggacgaaaccguyucuccagacucccgguucygurgugguuuguguaucccacrcrguuacuuccgguaacuuggaguuuguagcgacauaaguuucguccuaaaaggacucaucagguagccuaauagacuacgacuuaugcgguaggucgugaauacgcccggaguacaucuccaauggaacuaggagaccccuccggucguauccaaccucuguuucggcagaggauaccgurcuccacagcuggaaggcugugggcccgagagaagcgcucucucucgcccuucuacucccccucggcaugagauguccauaggccaggggugagauccaacgggaucacagcugaucuaagguggguaguaggggcacggguuuuaucacccccgugacgguc

SEQ ID NO: 475 - RNA, 432 nt, Artificial Sequence
  Synthetic Construct
graggcuauuagccuuccugaugaguccguuccaaggacgaaaccguuucuccagacucccgguucugaggugguuuguguaucccacucaguuacuuccgguaacuuggaguuuguguaayguaaguuucguccuaaaaggacucaucagguagccuaauagacuacgacuuaygcgguaggucgugaauaaggccggaguacauucccuauggaacuaagggaaccccuccggucuuauccaaccucuguuucggcagaggauaccgugcuccacagcuguaaggcugugggcccgagagaagcgcucucucucgcccuucugcucccccucggcauugagauguccauaggccaggggugagauccaacgggaucacagcugaucuaaggugggcagcagggguauggguuugucgacacccaacgguc

SEQ ID NO: 476 - RNA, 430 nt, Artificial Sequence
  Synthetic Construct
ggaggcuayuagccuuccugaugaguccguuccaaggacgaaaccgucucccagacucccggugucgaggugguuuguguaucccacucggcuacuyccgguaacuugggguuugugyygacauaaguuucguccuaaaaggacucaucagguagccuaauagacuacgacuuaugcrguaggucgugaauacgcccggaguacaucuccaauggacuaggagaccccuccggucguauccaaccucuguuucggcagaggauaccguacuccacagcuguaaggcugugggcccgagagaagcgcucucucucgcccuuyugcucccccucggcaugagauguccauaggccaggggugagaucaacgggaucacagcugaucuaaggugggcagcaggggcacggguuuugrgacccccgugacgguc

SEQ ID NO: 477 - RNA, 433 nt, Artificial Sequence
  Synthetic Construct
ggaggcuauuagccuuccugaugaguccguuccaaggacraaaccgucucuccagacucccgguucugguuggguuaguuauaccaacuaguuacuuccgguaacuuggaguuuguagcgacauaaguuucguccuaaaaggacuyaucagguagccuaauagacuacgacuuaugcgguaggucgugaauaaggccggaguacauucccaauggaacuagggaaccccuccggucuuauccaaccucuuuuucggaagaggauaccguacucccaagcuguaaggcuuggggcccgagagaagucgcucucucucgcccuauacauccucucaggcauagagaugaccauuugccuagggugagucuuuaaguagacacagccuaucuauggugauguacuggggcauggguuuagaccacccaugacgguc

SEQ ID NO: 478 - RNA, 353 nt, Artificial Sequence
  Synthetic Construct
guccguuccaaggaygaaaccguyucuccurayucccaguuuccruagguuaauuauccucayggauuacuucygguaacyaggaguuugugyrayguaaguuucguccuaaaaggacucaucagguagccuaauagacuacgacuuacryggurggucgugaauaagccggaguacauucccaauggaacuagggaaccccuccggucuuauccaaccucuguuucggcagaggayaccgugcuccacaguugaagacugugggcccgagagaagucgcucucucucgcccyauacauccucucaggcauagagaugaccauuugccuagrgugagucuuuaaguagacaca

SEQ ID NO: 479 - RNA, 423 nt, Artificial Sequence
  Synthetic Construct
ggaggcuauuagccuuccugaugaguccguuccaaggayraaacygucuyccaracucccgguucugguuggguuaguauaccaacuaguuacuuccgguaacyuggrguuugurygacauaaguuucguccuaaaggaucaucagguagccuauagacuacgacuuaugcgguaggucgugaauaagaacggaguacaucuccaauggaacuaggagaccccuccguucuuauccaaccucuguuucggcagaggauaccguacucccuagcuguaaggcuaggggccygagagaagucgcucucucucgcccuuccauccucucaggcauagagaugaccauuugccuaggguragucuuuaaguagacacagccuaucuauggcgruggauaggggcacggguuuaaccccaugacgguc

SEQ ID NO: 480 - RNA, 433 nt, Artificial Sequence; identical to SEQ ID NO: 64
  Synthetic Construct

SEQ ID NO: 481 - RNA, 406 nt, Artificial Sequence
  Synthetic Construct
cgggaacuuucuugagguuccuguggugcucaccugacccugcaggcaguaaaagaaaaaagaggcggcgggggaagaaguccuucagggauccccggggaaaccuggaggaagucgaggucgggggggacuagcuguucggucgccgcggaucacuggcguccagcggagaaacaggagcucgucuccuuccuuucgcugcuggcuccacauccgaucgucgcugarccccgcugcuggcuccacauccgaucgucgcugaagcgccacgcccccucgcccggagcuucucucuggagacuacccgguggaaacaacugaagcuucaaccccaaaccgcuuuucuuauaucuucucugcucuccgggcgagggugaaagcccuuggaacccuagauugggucccu

SEQ ID NO: 482 - RNA, 413 nt, Artificial Sequence; identical to SEQ ID NO: 66
  Synthetic Construct

SEQ ID NO: 483 - RNA, 396 nt, Artificial Sequence
  Synthetic Construct
guuccaaggacgaaaccgucucuccugacucccgguucaguggugguuuguguaucccacacuguuacuuccgguaacuaggaguuuguagcgacauaaguuucguccuaaaaggacucaucagguagccuaauagacuacgacuuaugcgguaggucgugaauagccggaguacauycycaauggaacuagrgraccccuccggucuauccaaccucuguuucggcagaggauaccguacuccauagcuguaaggcuaugggcccgagagaagucgcucucucucgcccuucacaucccucucggcaugagaugaccauaggccaggggugaguccuuaaguggacacagcugaucuaaggcgguguguaggggcacggguuuucaacccccaug

SEQ ID NO: 484 - RNA, 350 nt, Artificial Sequence
  Synthetic Construct
cggaacuaaacucgugguuccugugguucacaccugaccuccugacagraaagaaaaaagaagcggcucggaggagcgcuucagggauccccggggaaaccuggagcgaacuggcaaaaggcgguggggagugcccagcggccgacaggaguaauucccgccgaaacaggguuuucacccuuccuuucuucggguguccuuccucgcgcccgcaggccaccccucgccccuuugcgcugucgcuucggyuacuacccgguggaaacaacugaagcucccgagaaccgcuuuuucucuaucuuyugcuycggggcgaggguguuuagcccuuggaaccgcaguugguuccu

SEQ ID NO: 485 - RNA, 395 nt, Artificial Sequence
  Synthetic Construct
ggcaccugaygucgguguccugaygaagauccaugacaggaucgaaaccucuuccaguuucggcuugugcgggaguaaagcygcucucuccacagccucrucaggaaacccacuucaggucucgacuggaaggucguaaacuuccccucuaagcggaguagagguaaauaccuccguccaaccccgggaggaaagggguuggracccggaacagauccaguuccgguccuuuggaguccauuucucucguuggauauucuccucggagaagggagauggggccagucccaguygguucgcucucguagucacagccrcuggggaaccuaggcagauggcuggacggagucuuaguccacuccagaggaccuuggguuugaaacccccaagagguc

SEQ ID NO: 486 - RNA, 389 nt, Artificial Sequence
  Synthetic Construct
cucuuccaguuucggcuugugcgggaguaaagcuuygcucucuccacagccucrucaggaaacccacuucaggucucgacuggaaggucguaacuuccycucuaagcggaguagagguaaauaccuccguccaccccgggaggaaggggugggacccggagcagaucyagcuccgguccuuuggaguccauuucucycguuggauauucuccucggrgaagggagauggggccagucccagucgguucgcucucguagucacagccgcuggggaaccuaggcagaggcuggacggagucuuaguccacuccaaaggaccuuggguuyaacccccaagaggucggcaccugagucgguguccugaygaagauccaugacaggaucgaaac

SEQ ID NO: 487 - RNA, 393 nt, Artificial Sequence
  Synthetic Construct
ggcaccugacgucgguguccugaygaagauccaugacaggaucgaaaccucuuccaguuucggcuugugcgggaguaaacgaaagucucuccacagccucgucaggaaacccacuucaggucucgacuggaaggucguaaacuuccycucuaagcggaguagagguaauaccuccgucccaycccgggaggaaagggruggggacccggaacagauccaguuccgguccuuuggaguccauuuyucucguuggauauucuccucggagaagggarauggggccagucccaguygguucgcucucguagucacagccrcuggggaaccuaggcagaggcuggacggagucuuaguccacuccaraggaccuuggguuraaacccccaagagguc

SEQ ID NO: 488 - RNA, 396 nt, Artificial Sequence
  Synthetic Construct
cuucaggucucgacuggaaggucguuaacuuccccucuaagcggaguagagguaaauaccuccguccaaccccgggaggaaagggguugggacccggarcagauccagyuccgguccuuuggaguccauuucucucguuggauauucuccucggagaagggagauggggccagucccagucgguucgcucucguagucacagccgcuggggaaccuaggcagaaggcuggacggagucuuaguccacuccaaaggaccuuggguuuaacccccaagaggucggcaccugaygucgguguccugacgaagauccaugacaggaucgaaaccucuuccaguuucggcuugugcgggaguaaagcuuucgcucucuccacagccucgucaggaaaccca

SEQ ID NO: 489 - RNA, 395 nt, Artificial Sequence
  Synthetic Construct
ggcaccugacgucgguguccugaugaagauccaugacaggaucgaaaccucuuccaguuucggcuugugcgggaguaaagcuuucgcucucuccacagccucaucaggaaacccacuucaggucucgacuggarggucguuaaacuuccccucuaagcggaguagagguaaauaccuccgucccaccccgggagaaagggguggggacccggaacagcucuaguuccgguccuuuggaguccruuucuycguuggagauuycyycggraagggagayggggccagucccagucgguucgcucucguagucacagccgcuggggaaccuaggcagauggcuggacggagucuuaguccacuccaaaggaccauggguuuuaracccccaugagguc

SEQ ID NO: 490 - RNA, 396 nt, Artificial Sequence; identical to SEQ ID NO: 74
  Synthetic Construct

SEQ ID NO: 491 - RNA, 391 nt, Artificial Sequence
  Synthetic Construct
auccccggggaaaccugaggaagguggggaugacgucaaguccgcauggcccuuaugggcugggccacacacgugcuacaauggcaauuacaaugggaagcaaggcuguaaggcggagcgaauccggaaagauugccucaguucggauuguucucugcaacucgggaacaugaaguuggaaucgcuaguaaucgcggaucagcaugccgcggugaauauguacccgggcccuguacacaccgcccgucacacccugggaauugguuucgcccgaagcaucggaccaaugaucacccaugacuucuguguaccacuagugccacaaaggcuuuugguggucuuauuggcgcauaccacgguggggucuucgacuggggaagaagucuuaggg

SEQ ID NO: 492 - RNA, 389 nt, Artificial Sequence; identical to SEQ ID NO: 76
  Synthetic Construct

SEQ ID NO: 493 - RNA, 362 nt, Artificial Sequence
  Synthetic Construct
cgggaucuuucuugagguuccuguggugcucaccugacccugcaggcaggaaaagaaaaaagaggcggcggggaagaaguccuucagggauccccggggaaaccuggaggaagucgaggucggggggrcarcugcuucggucgccgcggaucacuggcguccagcggagaaacaggagcucgcuccuuccuuucgcugcuggcuccacauccgaucgucgcugaagcycrcgcccccucgcccggagcuucucucuggacuacccgguggaacaacugaagcuucaaccccaaccgcuuuucuuraucuucacugcucuccgggcgagggugaaagcccuyggaacccuagauugggucccu

SEQ ID NO: 494 - RNA, 219 nt, Artificial Sequence
  Synthetic Construct
rrgaarccucgarcyggcargrgcaggugugrarcgaacagaguaryccgggaacarycggyuycrggcyacrcccucgccccrgcauagrurgaraaagrgyucycgrgarcycuuuyucyaycgygyccggggcgagggygurgccygrarccgcrygguuycucgrryacucuguucgyucacaccugcycyugcrrgyaggycycgrggauaggg

SEQ ID NO: 495 - RNA, 380 nt, Artificial Sequence; identical to SEQ ID NO: 79
  Synthetic Construct

SEQ ID NO: 496 - RNA, 322 nt, Artificial Sequence
  Synthetic Construct
gggccugucuccuaagaugccuccgcuaguygagcggacuuggccucuuccgcccgaggaccuuuucuuucugaucuugcuuguuccaacgagccccgcgaguggaauccccggaaccccugcgaaaarrgguccucggaucauuuuccuugugguuccuguggyuacaccucggaaggccuccgcggaccugcagagaaaagaagaagggcccagaggggaaugagccucgucgucgacgaaggggugcauuccgaagagcuggccuggcgucguccggcucccucgaccgcaccggagcgcuagaaaagguccucggacu

SEQ ID NO: 497 - RNA, 364 nt, Artificial Sequence
  Synthetic Construct
ugggcaccaacuaggguuccugugguccaccgaaggccgcgaagcuaggaagaaaaaguagaaaagcuggguaagacucaccuggcgacucgucgucgacgaaggguccucagcagagcaccggcaggaggcgcuaugcaggaacgcuagggguccuccagcggaggacugaagaaacuccgguuucuucuuucacucuguagcuggaaucccuguugcgcuugcuggcgaaaccugcagggaagcuagcugggucccgcuagucgagcggacucgucccagcggucccaaccaguuuucuuuauccuauuuuucccugcgggcgcccggucgugguuacccuggaryycycuuuuggaggccc

SEQ ID NO: 498 - RNA, 230 nt, Artificial Sequence
  Synthetic Construct
rycgrcagagcgcyagyrrgarcgcuggccuycrgygrracargaaccyrguuucyyrycuyaryggaaccygygcgcyygcggraaaygargraarcurgyugggccyrgcgagyrcucgcyrggcccarcyagyuuycyucruuuyccgcrrgcgcgruyyrgyuacycuagyycycygyugrrgrcccyrgcrcyruaggyuccugyggurcuccraggcccugurr

SEQ ID NO: 499 - RNA, 357 nt, Artificial Sequence
  Synthetic Construct
ugggcuccaacuagugguuccugugguuucacaccgaagccgcgagcuuggaaagaaaagaaraaaagcuggguaagcucaccuggugacucgucgucgacgaaggguccucagcagagcaccggcaggcgcggcgagragaacgcuagccguccuccagyggagaacuggaaaagcuccggcuuuuccuuuuuucugurcuggaacgcgcuguugcgcucgcugccagaccugcagggaagcuagcugggucccgcuagucgagcggacucgucccagcggcccaaccaguuuuyuuuccuauuuuucccugcgagcgcccggucguggauaccuaggacccuuguuugggugccc

SEQ ID NO: 500 - RNA, 375 nt, Artificial Sequence; identical to SEQ ID NO: 84
  Synthetic Construct

SEQ ID NO: 501 - RNA, 373 nt, Artificial Sequence
  Synthetic Construct
cggaacuaaacucgugguuccugugguucacaccugacccugcagccaugcaaagaaaaaaagaugggcggargagcgcaagagcggucucaggagccccggggcaacucagaccgagcggggucggaggaucuucuggagcggggcccugcuucagacaggaguaauccccgcugaaacaggguuuucacccuuccuuucuucggguuuccuuccucuguugcggaccucgcccggagucuucucgaccagcgcagguucugacgcgaccgguggcaucaccgaguugcucaagccucaaccgccuuuuucucuauucurcgcuuggucuccgggcgaggguguuuagcccuuggaaccgcaguugguuccu

SEQ ID NO: 502 - RNA, 363 nt, Artificial Sequence
  Synthetic Construct
cgggacuuucuugagguuccuguggugcucaccugacccugcaggcayaaagaaaaaagaagcggcrrgggaagaaguccuucagggauccccggggaaaccuggaggaagucgaggucgggggagcggcugcuucggucgccgcggaucacuggcguccagcggagaaacaggagcucgucuccuuccuuucgcugcuggcuccacauccgaucgucgcugaagcgccucgccccucgcccggagcuucucucuggagacuacccgguggauacaacugaagcuucaacccaagccgcuuuuucuaucuucucugcucuccgggcgagggugaaagcccuuggaacccuagauugggucccu

SEQ ID NO: 503 - RNA, 366 nt, Artificial Sequence
  Synthetic Construct
cgggaucuuucuugagguuccuguggugcucaccugacccugcaggcagaaaaagaaaaagaggcgggggggaagaaguccuucagggauccccggggaaaccuggaggaagucgaggucggggggagcuucugccucggucgccgcggaucacuggcguccagcggagaaacaggagcucgcuccuuccuuucgcugcuggcuccacauccgaucgucgcugaggcguygccaccccucgcccggagcuucucucugguacuacccgguggauacaacugaagcuucaaccccaguaccgcuuuucuugaucucuacugcucuccgggcgagggugaaagcccucggaacccuagaugggucccu

SEQ ID NO: 504 - RNA, 363 nt, Artificial Sequence
  Synthetic Construct
ugggcuccaacuagugguuccurugguucacaccgaaggccgcgagcuggaaagaaaagauaraaagcuggguaagacucaccuggygacucgucgucgacgaaggguccucagcagagcaccggcaggcgcggcgagcggagaacgcuagcyguccuccaguggagaacuggaaaagcuccggcuuuuccuuuuuucuguaacuggaacgcgcuguugcgcucgcuagccagaccugcagggaagcuagcugggucccgcuagucgagcggacucgucccagcggucccaaccaguuuyuuuauccuauuuuucccugcgrgcgcccgguyguggaurccuaggauucccguuugggugccc

SEQ ID NO: 505 - RNA, 357 nt, Artificial Sequence
  Synthetic Construct
cggaacuaaacucgugguuccuguggucacaccugaccyygyrgcaaaaagaaaaaagaacgggaggragagcgcaagagcggucucaggagccccggggcaacucagaccgagcggggucuyggucgagggcgugcccuguucagacaggaguaaucccagcugaaacaggguuuucacccuuccuuucuucugguuuccuuccucugcuucagcggccucgcccggaaccucuugaccagcgcaggugcugacgcgaccgguggcaucaccgaguugcucragccucaaccuccuuuuucucuaucurcuugcuccgggcgaggguguuuagcccuuggaaccgcagugguuccu

SEQ ID NO: 506 - RNA, 373 nt, Artificial Sequence; identical to SEQ ID NO: 90
  Synthetic Construct

SEQ ID NO: 507 - RNA, 373 nt, Artificial Sequence; identical to SEQ ID NO: 91
  Synthetic Construct

SEQ ID NO: 508 - RNA, 369 nt, Artificial Sequence
  Synthetic Construct
gaucacuggcguccagcggagaaacaggagcucgacuccuuccuuucgcugcuggcuccacauccgaucgucgcugaagcgycrcgcccccucgcccggagcuucucucuggcuacuacccgguggauacaacugaagcuucaaccccaaaccgcuuuucuugaucuucacugcucuccgggcgagggugaaagcccucggaacccuagauugggucccucgggaucuuucuugagguuccuguggugcucaccugacccugcaggcaggaaaagaaaaaagaggcggcgggggaagaaguccuucagggauccccggggaaaccuggaggaagucgaggucgggggggaucaacagcuucggucgccg

SEQ ID NO: 509 - RNA, 194 nt, Artificial Sequence
  Synthetic Construct
ycgrggycgrrgrrcuucuccycrcrcgraucyucccgcrgrgaargagcccccuyuycygggyccaycaguycgrgyucrcccucgcccggagcyucuggyurcyaccggurgrcragaagcuycrrccycagyrcguyuycuucucyrcugcccrggrycccggrccagrgrgyycccgacuuycyyagguu

SEQ ID NO: 510 - RNA, 200 nt, Artificial Sequence
  Synthetic Construct
auccccggggaaaccuggaggaagucgaggucgggggggacagcugcuucggucgccgcggaucacuggcguccagcggagaaacaggagcucgucuccuuccuuucgcugcuggcuccacauccgauygucgcugaagcgcccgcccccucgcccggagcuucucucuggagacuacccgguggaaacaacugaagcuu

SEQ ID NO: 511 - RNA, 363 nt, Artificial Sequence
  Synthetic Construct
ggaaaccuggaggaagucgaggucgggggggaacgcugcuucrycrcgcgaucacuggcguccagcggagaaacaggagcucgucuccyuccuuucgcugcuggcuccacauccgaucgucgcugargcycrcgcccccucgcccggagcuucucucuggcuacuacccgguggauacaacugaagcuucaaccccaaaccgcuuuucuuraucuucacugcucuccggrcgagggugaargcccucggarcccuagaugggucccucgggaucuuucuugagguuccuguggugcucaccugacccugcaggcaggaaaagaaaaaagaggcggcggggaagaaguccuucaggguccccrr

SEQ ID NO: 512 - RNA, 372 nt, Artificial Sequence; identical to SEQ ID NO: 96
  Synthetic Construct

SEQ ID NO: 513 - RNA, 363 nt, Artificial Sequence
  Synthetic Construct
ugggcuccaacuagugguuccugugguuucacaccgaaggccgcgaagcuuggaaaraaaagaaraaagcuggguaagacucaccugggacucgucgucgacgaaggguccucagcagagcccggcaggcgcggcgaagcggagaacgcuagccguccuccaguggagaacuggaaaagcuccggcuuuuccuuuuuucuruaacuggaacgcgcuguugcgcucgcuagccagaccugcagggaagcurgcugggucccgcuagucgagcggacucgucccrgcggucccaaccaguuuuuuuycyauuuucccugcgagcgcccggucguggauaccuaggayucccuuguuugggugccc

SEQ ID NO: 514 - RNA, 358 nt, Artificial Sequence
  Synthetic Construct
ygugguuccaauggugcaccccugaccugcaagcaaaagaaaaarryggggcggcggcacaacgcuucagggauccccggggaaaccuggagcgaacucggcaaggaggcyuggcgguaggugcgcggagucgaccgcguaaagaccggagaacucccgcggcggaagaaacaggagcucgcuccuuccuuucugucgcccgcaggacucayyyggcagcggcgucuucuccgcacccycgcccgcuuccgcgcuggucgcucuggcuacuacccgguggaacaacuguagcuugagcyygcccgcccuuuuucuuuyuucugcugcgcggcgagggugggcuucuaaaggaaacccu

SEQ ID NO: 515 - RNA, 359 nt, Artificial Sequence
  Synthetic Construct
cggaacuaaacucgugguuccugugguucacaccugacccugcagccaugcaaagaaaaaagaacgggaggaagagcgcaagagcggucucaggagccccggggcaacucagaccgagcggggaucgcggaccgagggcggaagccugcuucagacaggaguaaucccgygaaacaggguuuucacccuccuuucuucggguuuccuuccucugcuugcggccucgcccggagucuucgaauccagcgcagguucugacgcgaccgguggcacrccgaguygcucagccucaaccuccuuuuycuucuagcuuggucuccgggcgagggugyuuagcccuggaaccgcaguugguuccu

SEQ ID NO: 516 - RNA, 360 nt, Artificial Sequence
  Synthetic Construct
cgggaucuuucuugagguuccuguggugcucaccugacccugcaggcagaaaagaaaaaagaggcgggggggaagaaguccuucagggauccccggggaaaccugaggaagucgaggucggggggagcaacugccucggucgccgcggaucacuggcguccagcggagaaacaggagcucgcuccuuccuuucgcugcuggcuccacauccgaucgucgcugaggcyugcgcgccccucgcccggagcuucucucuggacuacccgguggaacaacugaagcuucaaccccguaccgcuuuucuugaucurcugcucuccgggcgagggugaaagcccucggaacccuagaugggucccu

SEQ ID NO: 517 - RNA, 40 nt, Artificial Sequence
  Synthetic Construct
agggygraarccugyuucagrggauauccycugaarcagg

SEQ ID NO: 518 - RNA, 335 nt, Artificial Sequence
  Synthetic Construct
agucgaggucggggggrrcracugccucggucgccgcggaucacuggcguccagcggagaaacaggagcucgcuccuuccuuucgcugcuggcuccacauccgaucgucgcugaggcyugcgcccccucgcccggagcuucucucuggcuacuacccgguggauacaacugaagcuucaaccccraccgcuuuucuurucuyacugcucuccgggcgagggugaaagcccucggaacccuagaugggucccucgggaucuuucuugagguuccuguggucucaccugacccugcaggcaggaaaagaaaaaagaggcgggggaagaaguccuuca

SEQ ID NO: 519 - RNA, 359 nt, Artificial Sequence
  Synthetic Construct
ggaacuaaacycgyggyyccgygguucacaccugacccugcagccaugcaaagraaaaagaacgggagrragagcgcaagagcggucucaggagccccggggcaacucagaccgagcggggucuugaccaguggcgagcgcccuguucagacaggaguaaucccgygaaacaggguuuucacccuuccuuucuucugguuuccuuccucugcuucagcggccucgcccggagucuugaccagcgcagguucugacgcgaccgguggcaucaccgaguucgcucaarccucycuccuuuuucuyaucuagcuuggucuccgggcgaggguguuuagcccuuggaaccgcaguugguuccy

SEQ ID NO: 520 - RNA, 251 nt, Artificial Sequence
  Synthetic Construct
uggcguccagcggagaaacaggagcucgacuccuuccuuucgcugcuggcuccacauccgaucgucgcugaggccugcgcgccccucgcccggagcuucucucuggcuacuacccgguggauacaacugaagcyucaaccccguaccgcuuuucuuauyyyacugcucuccgggcgagggugaaagcccucggaacccuagagugggucccucgggaucuuucuugagguuccuguggugcucaccugacc

SEQ ID NO: 521 - RNA, 369 nt, Artificial Sequence; identical to SEQ ID NO: 105
  Synthetic Construct

SEQ ID NO: 522 - RNA, 369 nt, Artificial Sequence; identical to SEQ ID NO: 106
  Synthetic Construct

SEQ ID NO: 523 - RNA, 369 nt, Artificial Sequence; identical to SEQ ID NO: 107
  Synthetic Construct

SEQ ID NO: 524 - RNA, 346 nt, Artificial Sequence
  Synthetic Construct
cggaucuucuugcuugugguuccugugguucaccucggaaggccccgcggccugcararaagaagauggggcagagggggarugagccucgucgucgacgaaggggugcayuccgagugccugagcuggucgacguccagcucccucgggaccygcugcucugggcggaagagucuucugacyuucuagccuauucagcucgcguccuugaggccuggcguaacgcggcucuugccaccggagccuccgcuagucgagcggacuuggucucyuccgcccaaagcccuuuuucuucugcuuguccaacgcgccccgcgagugcaauccccggaacccccgcaagagg

SEQ ID NO: 525 - RNA, 353 nt, Artificial Sequence
  Synthetic Construct
caccucggaaggccgccgcggaccugcararaagaagauaggggcagagggggugagccucgucgucgacgaaggggugcaccgagugccugagcugrucgacguccagcucccucgggaccacgcugcucugggcggaagagucuucugacyuucuagccuaucagcuucgcguccuugaggccuggcguaacgcggcucuugccaccgggccuccgcuagucgagcggacuuggucucuuccgcccaaagcccuuuuucuuucaugcuuguccaacgcgccccgcgagugcaauccccggaacccccgcuaagaggucuccggaucuucuugccugugguuccugugguuu

SEQ ID NO: 526 - RNA, 367 nt, Artificial Sequence; identical to SEQ ID NO: 110
  Synthetic Construct

SEQ ID NO: 527 - RNA, 229 nt, Artificial Sequence
  Synthetic Construct
cauccggrcyygaragucrguugaaagaaaarrrgugggcrgargrgryyrrcccucgacarggrgcrcygrgcgagcygyacgycrgcyycgrrcaygcugyggyrgaaggucgaccuucyrccragccrguccygrrgcygrcgurcrgcucgcycrgrygcyccyugucgagcacuycycyucygcccaycyuyuuyyuurcgrucgcyuuccragygyccggaug

SEQ ID NO: 528 - RNA, 351 nt, Artificial Sequence
  Synthetic Construct
cggaucuucuugcuugugguuccugugguuucaccucggaaggccgccgcggaccugcagaraagaagauggggcagaggggguugagccucgucgucgacgaaggggugcrrccgagugccugagcuggucgacguccagcucccucgggaccacgcugcucugggcggaagagucuucugacacuucuagccuauucauucgcguccugaggccuggcguaacgcggcucuugccaccgggccuccgcuagucgagcggacuuggucucuuccgcccaaagcccuuuuucuuucaucgcuuguuccaacgcgccccgcgagugcaauccccggaacccccgcuaagagg

SEQ ID NO: 529 - RNA, 283 nt, Artificial Sequence
  Synthetic Construct
cuggggaauucucgaguugccgcauacuuaccugagaaaggagccccggggcaacucuucucagaauccagcgagaggcguaggagagagggccgcggugcucuggaguagaggcuucuucgaaacaccaucgaucgucccuucuucuuuuaccaggaugccuccgcuagucgagcggacuuggucucuuccaccgguggcaucaccucucgguucgucuuccaaccugcuuuuuuucuaucugagccucugccgcggauccucucuugagccccuuaagagg

SEQ ID NO: 530 - RNA, 185 nt, Artificial Sequence
  Synthetic Construct
acuuggucucuuccgcccaaagcccuuuyucuuucauagcuuguuccaacgcgccccgcgagugcaauccccggaacccccgcuaagaggucuccggaucuucuugcuugugguuccugugguuucaccucggaaggccgccgcggaccugcaragaagargauaggggcagagggggugagcyc

SEQ ID NO: 531 - RNA, 367 nt, Artificial Sequence; identical to SEQ ID NO: 115
  Synthetic Construct

SEQ ID NO: 532 - RNA, 354 nt, Artificial Sequence
  Synthetic Construct
cggaucauuuuccyugugguuccugugguuacaccucggaaggccuccgcggaccugcagagaaaagaagaagggcccagaggggaaugagccucgucgucgacgaaggggugcauuccgaagagcuggccuggcgucguccggcuyccucgaccgcaccggagcgcuagaaaagguccucggacuuucuucuaucuccgaagycgguuugaggcccggcgaaacgcgggccugucuccuaagaugccuccgcuagucgagcggacuuggccucuuccgcccgaggaccuuuucuuucuraucuugcuuguuccaacgagccccgcgaguggaauccccggaaccccuggaaaa

SEQ ID NO: 533 - RNA, 365 nt, Artificial Sequence
  Synthetic Construct
ucuccggaucuucuugcuugugguuccugugguucaccucggaaggccccgcggaccugcaragaagaagauaggggcagaggggguucgagccucgucgucgacgaaggggugcgaaccgagugccugagcuggucgacguccgacucccucgagaccugcugcucugggcggaagagucuucgacuccuucuuugccuauucagcuucgcgucucugaggcucggcguaacgcggcucuugccaccagguugccuccgcuagucgagcggacuuggucucuuccgcccraagcycuuuuucuuucuaaucagcuuguuccaacgcgccccgcgaguacaauccccggaacccccgcuaagagg

SEQ ID NO: 534 - RNA, 355 nt, Artificial Sequence
  Synthetic Construct
cggaucacuuuccugugguuccugugguurcaccucggaaggccgccgcggaccugcaaagaagaagauaggggcagagggggagugagccucgucgucgacgaaggggugcacuccaaagcuccgaacuggcgucguccggcucuccucggagccucgcugcucugggcggaagagucuucugacuuuucuagccuauucagcauugcgcucuugaggcccggcgaaacgcggyucuyuaggaugccuccgcuagucgagcggacuuggucucuuccgcccaaagcccuuuuucucaacugagcuuguuccaacgcgccccgcgaguggaaucyccggaaccccugcaaagagg

SEQ ID NO: 535 - RNA, 366 nt, Artificial Sequence; identical to SEQ ID NO: 119
  Synthetic Construct

SEQ ID NO: 536 - RNA, 214 nt, Artificial Sequence
  Synthetic Construct
ccgcuagucgagcggacuuggucucuuccgcccaaagcccuuuuucuuucaacugagcuuguccaacgcgccccgcgagugaauccccggaaccccygcaagaggucyycggauccuucyugugguuccugugguucaccucggaaggccgccgcggaccugcaaagaagaagauaggggcagagggggagugagccucgucgucgacgaaggg

SEQ ID NO: 537 - RNA, 269 nt, Artificial Sequence
  Synthetic Construct
cugugguuucaccucggaaggccgccgcggaccugcaaagaagaagauaggggcagagggggagugagccucgucgucgacgaaggggugcacuccgagugccugagcuggucgacguccagcucccucgggaccugcugcucugggcggaagagucuucugacacuucuagccuauucagcuucgcguccuugaggccuggcguaacgcggcucuugccaccaggagccuccgcuagucgagcggacuuggucucuuccgcccaaagc

SEQ ID NO: 538 - RNA, 366 nt, Artificial Sequence; identical to SEQ ID NO: 122
  Synthetic Construct

SEQ ID NO: 539 - RNA, 366 nt, Artificial Sequence; identical to SEQ ID NO: 123
  Synthetic Construct

SEQ ID NO: 540 - RNA, 358 nt, Artificial Sequence
  Synthetic Construct
cgggaacuuucuugagguuccuguggugcucaccugacccugcaggcaucaagaaaaaagauggcgcggaggagaagaaguccuucagggauccccggggaaaccuggaggaagucgaggucgggggcuucggacuacuccuucgugagacaggaguaauycccgcygaaacaggguuuucacccuuccuuucuucggguuccuuccucucgccuggagaggucuucggcccucgcccggagcuucucucuggagacuacccgguggaacaacugaagcuucrcycuccgcgcuuuuucucuaucuuuguugcucuccgggcgagggugaaagcccguggaacccurgargagucccu

SEQ ID NO: 541 - RNA, 364 nt, Artificial Sequence; identical to SEQ ID NO: 125
  Synthetic Construct

SEQ ID NO: 542 - RNA, 354 nt, Artificial Sequence
  Synthetic Construct
cgggaacuuuuugagguuccuguggugcucaccugacccugcaggcaucaagaaaaaagaauggcgccrggagaagaaguccuucagggauccccggggaaaccuggaggaagucgaggucgggggcuucggaucauuccugguugagacaggaguaaucccagcugaaacaggguuuucacccuuccuuucuucugguuuccuuccucucgccggaaggucuucggccucgcccggagcuucucucuggagacuacccgguggaaacaacugaagcuucgacuccacggccuuuuucucuaucuuuguugcucugggcgagggugaragcccguggacccugraarggucccu

SEQ ID NO: 543 - RNA, 348 nt, Artificial Sequence
  Synthetic Construct
cgggacuuucugagguuccuguggugcucaccugacccugcaggcaucaagaaaauaggagcgggaggaagaaguccuucagggauccccggggaaaccuggaggaagucgaggucgggggcuucggaucauuccugguugagacaggaguaaucccagcgaaacaggguuuucacccuuccuuucuucugguuuccuuccucucgccggaaggucuucggcccucgcccggagcuucucucuggagacuacccgguggaaacaacugagcuuccacucccgcucuuuuucucuaucuuuguugcucuccgggcgagggugaaagcccguggaacccugraggucccu

SEQ ID NO: 544 - RNA, 364 nt, Artificial Sequence; identical to SEQ ID NO: 128
  Synthetic Construct

SEQ ID NO: 545 - RNA, 364 nt, Artificial Sequence; identical to SEQ ID NO: 129
  Synthetic Construct

SEQ ID NO: 546 - RNA, 364 nt, Artificial Sequence; identical to SEQ ID NO: 130
  Synthetic Construct

SEQ ID NO: 547 - RNA, 266 nt, Artificial Sequence
  Synthetic Construct
auccccggggaaaccuggagcgaacuggcaaaaggcgguggggagugcccagcggccgacaggaguaauucccgccgaaacaggguuuucacccuuccuuucuucggguguccuuccucgcgcccgcaggaccaccccucgccccuuugcgcugucgcuucggcuacuacccgguggaaacaacugaagcucccgagaaccgcuuuuucucuaucuuyuugcuucggggcgaggguguuuagcccuuggaaccgcaguugguuccu

SEQ ID NO: 548 - RNA, 358 nt, Artificial Sequence
  Synthetic Construct
agggacuccuuccaggguuccacgggcuuucacccucgcccggagagcaacaaagauagagaaaaagcgcgagagaguugaagcuucaguuguuuccaccggguagucuccauaagaagcuccggcaaaggccgaagaccuuccggcgagaggaaggaaaccagaagaaaggaaggaugaaaauccuguuucugcugggauuacuccugucucaaccaggaaugauccgaagcccccgaccucgacuuccuccagguuuccccggggaucccgaaggacuucuucuccuccgcgccaucuuuuuucuugaugcyugcagggucaggugagcaccacaggaaccucaagaaaguucccg

SEQ ID NO: 549 - RNA, 351 nt, Artificial Sequence
  Synthetic Construct
cggaucauuuucuugugguuccugugguacaccuccgaaggccuaggcgagaagagaguaaauagaggagagagcuuaccuggguccuggcagcgcugcaacggaauucagggcguuggccccgruggaguuccgaugagcaccgggagaagagacugucucgcagacuagaagucucuucuuuucugcuuugagacuucuucuccguaggacucuucggcucuucgcggguacccagcaaccugcucccacgcggcgauugcaaaggauccgguucgcucuccuuuucuuuuuuaccurcuucagccgaccccggggagugaaucaccugggaccgcgaaaagcgauccu

SEQ ID NO: 550 - RNA, 348 nt, Artificial Sequence
  Synthetic Construct
auccccggggaaaccuggagcgaacuggcaargrcgguggggagugcccagcggccgacaggaguaauucccgccgaaacaggguuuucacccuuccuuucuucggguguccuuccucgcccgcaggaccaccccucgccccuuugcgcugucgcuucgguayuacccgguggaaacaacugaagcucccgagaaccgcuuuuucucuaucuuyuugcuucggggcgaggguguuuagcccuuggaaccgcaguugguuccucggaacuaaacucgugguuccugugguucacaccugaccuccugagcaraaaagaaaaaagaagcggcucggaggagcgcuucagg

SEQ ID NO: 551 - RNA, 361 nt, Artificial Sequence; identical to SEQ ID NO: 135
  Synthetic Construct

SEQ ID NO: 552 - RNA, 345 nt, Artificial Sequence
  Synthetic Construct
cgggaucuuuuccuugugguuccugugguucacaccugaccuccgcycagraaagaaaaaraaggcggcucggaggagcgcuucagggauccccggggaaaccuggagcgaacuggcaaaggaggcggcuggggagucuyccagacaggaguaauccccgcugaaacagguuuucaccuuccuuucuucggguuuccuuccucugrgucgacacccucgcccgccuyyyugcgcugucgcuucgguacuacccgguggaaacaacugaagcucccgagaaccguuuuucucuaucuugcugrcgcggggcgaggguggaaagcccuggaacccyrragggucccu

SEQ ID NO: 553 - RNA, 348 nt, Artificial Sequence
  Synthetic Construct
cggaacuaaacucgugguuccugugguucacaccugaccuccuggcagaaaagaaaaaarruaggcggcucggaggagcgcuucagggauccccggggaaaccuggagcgaacuggcaaaaggcggcagggagcuuguggaaggcgaaacaggaguaaccggagaaacaggruuuucayccuuccuuucuycgyuuccuuccuuugcgcgccacucgaccccucgcccccuugcgcugucgcuucggcacuacccgguggaacaacugaagcucccgagaaccgcuuuuucucuaucuugcugcuccggggcgaggguguuuagcccuuggaaccgcaguugguuccu

SEQ ID NO: 554 - RNA, 360 nt, Artificial Sequence; identical to SEQ ID NO: 138
  Synthetic Construct

SEQ ID NO: 555 - RNA, 190 nt, Artificial Sequence
  Synthetic Construct
auccccggggaaaccuggagcgaacuggcaaaaggcgcaggragcuugugrraggcgaaacaggagaucccgyguagaaacaggruuyyyccuuccuuucuucugcgguuuccuuccuuugcgcgccacucgaccccucgcccccuugcgcugucgcuucggcaacuacccgguggaaacaacugaagcu

SEQ ID NO: 556 - RNA, 360 nt, Artificial Sequence; identical to SEQ ID NO: 140
  Synthetic Construct

SEQ ID NO: 557 - RNA, 359 nt, Artificial Sequence; identical to SEQ ID NO: 141
  Synthetic Construct

SEQ ID NO: 558 - RNA, 194 nt, Artificial Sequence
  Synthetic Construct
gggauccccggggaaaccuggagcgaacuggcaaaaggccgguggggagugccucgcggccracaggaguaauuccugcygaaacaggguuuucacccuuccuuucuucagguuuccuuccuygcgcccgcaggaucaccccucgcccccuugcgcugucgcuucggcuacuacccgguggaacaacugaagcu

SEQ ID NO: 559 - RNA, 357 nt, Artificial Sequence
  Synthetic Construct
cggaacuaaacucgugguuccugugguucacaccugaccucugrgcagaaaagaaaaaagaaggcggcucggaggagcggucucaggagccccggggcaacucagaccgagcggggucucguggucgagggcguagccccuguucagacaggaguaaucccagcugaaacaggguuuucacccuuccuuucuucugguuuccuuccucugcuucagcggccucgcccggaaccucuugaccagcgcaggugcugacgcgaccgguggcaucaccgaguugcucccgagaaccgcuuuuucucuaucuuacuugcyucgggcgaggguguuuagcccuuggaaccgcaguugguuccu

SEQ ID NO: 560 - RNA, 356 nt, Artificial Sequence
  Synthetic Construct
ggagaaaacuccgugugguuccuguguggcaccccucccgcuacgggcuuuaaaaagaaagcagggaaggguacuuaccugucgucgucgacgaaggcugguaagcagagcccguugaagcucccgugacgagcgucgucggauggccuccgagacgagcgauggacaguggagcuyygcucucaccagucgcgugyugacucucgcgccuacccgaacacggcuguucygagggcgcygagggccgagcuucccgccgcuagucgagcggacycugguaguucccccgaccugccuauuuuuguagccuagggagaguggagucaccaggguaaaacacgauugguguuuucccc

SEQ ID NO: 561 - RNA, 357 nt, Artificial Sequence
  Synthetic Construct
auccccggggaaaccuggagcgaacuggcaaaaggcgcgguggggagugccucgcggccracaggaguaauuccugcugaaacaggguuuucacccuuccuuucuucagguuuccuuccuygcgcccgcaggaucaccccucgcccccuugcgcugucgcuucggcuacuacccgguggaaacaacugaagcucccgagaaccgcuuuuucucuaucuugcugguaycggggcgaggguguuuagcccuuggaaccgcaguugguuccucggaacuaaacucgugguuccugugguucacaccugaccuccuuccagaaaagaaaaaagauaggcggcucggaggagcgcuucaggg

SEQ ID NO: 562 - RNA, 120 nt, Artificial Sequence
  Synthetic Construct
gcaarrgggygrggrgugycyygcggcgaggarraccuggaaaaggguacccuuuuccagguyyuccucgccgcrrgrcacyccycrccgcgccuuygcyaucguccgguuccycggggu

SEQ ID NO: 563 - RNA, 348 nt, Artificial Sequence
  Synthetic Construct
cgggacuuacuugugguuccuguggugcacuccugacccugcugcuuraagaaaaagaaaurrggcgaagaaguccuucagggauccccggggaaaccuggaggaaguccgacgagaucgcggcuggggcuuaggaccccacuccugcgagacaggaguaauccuaaacaggguuuucacccuuccuuuaguuuccuuccucuccuggagaggucuucugcccuagcccggucuucgaagcuuccuuuggcacuacccgguggaaacaacugaagcuucaacgccuuuuuuuccaucuucuuuagcaccgggcuagggaguaagcccguggaaccurgyuuguucccu

SEQ ID NO: 564 - RNA, 339 nt, Artificial Sequence
  Synthetic Construct
ggggaaaccuggaggaaguccgacgagaucgcggcuggggcuugaccccacuccugcgagacaggaguaaccuaaacaggguuuucacccuuccuuuaguuccuuccucuccuggagaggucuucugcccuagcccggucuucgaagcuuccuuuggcuacuacccgguggaaacaacugaagcuucaacgccuuuuuuycaucuucuuuagcaccgggcuagggaguaagcccguggaaccuuaguuuuguucccucgggacuuacuugugguuccuguggugcacuccugacccugcugcuauaaagaaaaagaaaurrggyaaaaguccuucaggg

SEQ ID NO: 565 - RNA, 356 nt, Artificial Sequence; identical to SEQ ID NO: 149
  Synthetic Construct

SEQ ID NO: 566 - RNA, 353 nt, Artificial Sequence
  Synthetic Construct
aaagaaaugaggcgaagaaguccuucagggauccccggggaaaccuggaggaaguccgacgagaucgcggcuggggcuuaggaccccacuccugcgagacrggaguaauccuaaacaggguuuucacccuuccuuuaguuuccuuccucuccuggagaggucuucugcccuagcccggucuucgaagcuuccuuuggcuayuacccgguggaaacaacugaagcuucaacgccuuuuuuuccaucuucuuuagcaccgggcuagggaguaagcccguggaaccuuaguuuuguucccucgggacuuacuugugguuccuguggugcacuccugacccugcugcuuugaaagaa

SEQ ID NO: 567 - RNA, 354 nt, Artificial Sequence; identical to SEQ ID NO: 151
  Synthetic Construct

SEQ ID NO: 568 - RNA, 336 nt, Artificial Sequence
  Synthetic Construct
cucaaaaguuucguugcauyucagcaacucaucagugggcuuagcccagacuuuugrgagauraccucucagccccuccaccuuggggugcccuauccgagcacugcaguuucgguagaaaggcuaagaccucgcaaugagguaaggugggacuuuuccuucggaaccaagcgguugguuccgaggggggugugauccagguaccgccguagaaacuggauuacgacgucuacccgggauucaaacccgguccccuccagaagugaauucuggagaagagucugugcuaagcacacugaygagucucugaraugagacgaaacucuuyaruuyguy

SEQ ID NO: 569 - RNA, 351 nt, Artificial Sequence
  Synthetic Construct
cgggacuuacuugugguuccuguggyrcacuccugacccugcugcuuugaagaaaaagaaaugaccgaagaaguccuucagggauccccggggaaaccuggaggaaguccgacgagaacgcggcuggggcuuagggccccacuccugcgagacaggaguaruccuaaacrggguuuucacccuuccucuaguuuccuuccucuccuggagaggucuucugcccuagcccgguyyucgaagcuuccuuuggcaacuacccgguggaaacaacugaagcuucaacgccuuuuuuyccaaucuucuuuagcgccgggcuagggaguaagcccguggaaccuugguuuguucccu

SEQ ID NO: 570 - RNA, 354 nt, Artificial Sequence; identical to SEQ ID NO: 154
  Synthetic Construct

SEQ ID NO: 571 - RNA, 203 nt, Artificial Sequence
  Synthetic Construct
yuguygrygacgcrrgurcuycgagaagauccauyccgarccyyyyagyuycgucgagacracgguraguuucccuccyucugyuuuuauyuuraaggggrugccycaggaacacryggguuuccycgrggaaacccguguuccygrggccaycccyucyrrarauaaaarcagarggaggraacuyaccugycgguygygra

SEQ ID NO: 572 - RNA, 325 nt, Artificial Sequence
  Synthetic Construct
cauaaguuucgccgyruyucarcggcucaucagugggcuagcccagacuuaugagagaruraccucucagccccuccaccuuggrgugcccuauucggagcacugcaguucccgauagaaaggcuaagcaccucgcaaugaguaaggugggacuuuuccuucggaaccaagcgguugguuccgaggggggugugauccagguaccgccguagaaacuggauuacgacgucuacccgggauucaaacccgguccccuccagaagugauucuggagaagagucgygcuagcrcacugaugagucuyugarauragacgaaacucuuy

SEQ ID NO: 573 - RNA, 338 nt, Artificial Sequence
  Synthetic Construct
cucaaaaguuucgyygcauyucagcrrcucaucagugggcuuagcccagacuuuugagagurraccucucagccccuccaccuuggggugcccuauccgagcacugcaguuucgguagaaaggcuaagaccucgcaugagguaaggugggacuuuuccuucugaaaccaagcgguugguuccgaggggggugugauccagguaccgccguagaaacuggruuacgacgucuacccggrauucaacccgguccccuccagaagugaauucuggagaagagucugcgcuaagcgcacugaygagucucugaraugagacgaaacucuuyaraacuuuugu

SEQ ID NO: 574 - RNA, 333 nt, Artificial Sequence
  Synthetic Construct
yucaaaguuucgyygcauccagcrrcucaucagurggcuaagcccagacuuugagagaauaacaccucucagccccuccaccuuggggugcccuaucggacacugcaguuycgaurgaaaggcuaagcacucgcaaugagyaaggugggacuuuccuucggraccaagcgguugguuccgaggggggugugauccagguaccgccguagaaacuggauuacgacgucuacccgggauucaaacccgguccccuccagaagugauucuggaraagagucgygcuuagcrcacuaygagucucugaaugagacgaaacucuucragagcuuguuc

SEQ ID NO: 575 - RNA, 351 nt, Artificial Sequence; identical to SEQ ID NO: 159
  Synthetic Construct

SEQ ID NO: 576 - RNA, 340 nt, Artificial Sequence
  Synthetic Construct
cycaaaaguuucguugcauyucagcaacucaucagugggcuuagcccagacuuuugagagaurraccucucagccycuccaccuuggggugcccuauccgarcacugcaguuucgguagaaaggcuaagaccucgcaaugagguaaggugggacuuuuccuucggaaccaagcgguugguuccgaggggggugugauccagguaccgccguagaaacuggauuacgacgucuacccgggauucaaacccgguccccuccagaagugaauucuggagaagagucugugcuaagcacacugaygagucucugaraugagacgaaacucuucayuyguucrar

SEQ ID NO: 577 - RNA, 323 nt, Artificial Sequence
  Synthetic Construct
cauaaguuucgccuruuucaargcucaucagugggcuagcccagacuuaugrgaguaaaccucucagccccuccaccuuggrgugcccuauucggagcacugcrguucccgauagaaaggcuaagcaccucgcaaugaguaaggugggacuuuuccuucggaacyaagcgguugguuccgaggggggugugauccagguaccgccguagaaacuggauuacgacgucuacccgggauucaaacccggyccccuccagaagugauucuggagaagagucugygcuagcrcacugaugagucuuugaaauaagacgaaacucuuc

SEQ ID NO: 578 - RNA, 350 nt, Artificial Sequence; identical to SEQ ID NO: 162
  Synthetic Construct

SEQ ID NO: 579 - RNA, 325 nt, Artificial Sequence
  Synthetic Construct
cucaaaguuucgccuaucucaaggcucaucagugggcuagcccagacuuuugrgagauuaagaccucucagccccuccaccuuggggugcccuauycgrrcacugcaguuycragaaaggcuaagaccucgcaaugagguaaggugggacuuyuccuucggaaccaagcgguugguuccgaggggggugugauccagguaccgccguagaaacuggguuacgaygucuacccgggauucaaacccgguccccuccagaagugauucuggagaagagucugugcuagcacacugaygagucuyugaraugagacgaaacucuuyrr

SEQ ID NO: 580 - RNA, 322 nt, Artificial Sequence
  Synthetic Construct
ucaaaaguuucgccgcauyucagcggcucaucagugggcuagcccagacuuuugrgaruaaaaccucucagccccuccaccuuggggugcccuaucggrcacugcaguyycgaurgaaaggcuaagacucgyaaugaguaaggugggacuuuuccuucggaaccaagcgguugguuccgaggggggugugauccagguaccgccguagaaacuggauuacgacgucuacccgggauucaaacccgguccccuccagaagugauucuggagaagagucgygcuagcrcacugaygagucucugaraugagacgaaacucuuca

SEQ ID NO: 581 - RNA, 346 nt, Artificial Sequence
  Synthetic Construct
cycauaaguuucgycgcauyucagcggcucaucagugggcuuagcccagacuuaugagaguuaagaccucucagccccuccaccuuggggugcccuaaccggacacugcaguuucggaagaaaggcuaagcaccucgcaaugagguaaggugggacuuuuccuucuggaaccaagcgguugguuccgaggggggugugauycagguaccgccguagaaacugrruuacgacgucuacccgggauucaaacccgguccccuccagaagugauucuggaugaagagucugcgcuaagcgcacugaugagucucugaraugagacgaaacucuucgguayaraarurua

SEQ ID NO: 582 - RNA, 336 nt, Artificial Sequence
  Synthetic Construct
ccggauucuucuaagggugccuguggugccucccccgaagcccgcyuagggaaaaagaaaggggaagcaagcaucuccuguucagggauccccggggaaaccugracagaycgggcggagaagcgccggcgggccgucuucugacaggaguaaucccgyraaggguuuucacccuuccuuucuucggguuuccuuccucagucgaccggccgcgucggccuucucgcgcacucuguycggacuacccgguggauacaacugacagaggugcuuuuucuuccacccrcuucuaccgacgcggccgggagugaagcuacccgggacccgagaggaucu

SEQ ID NO: 583 - RNA, 349 nt, Artificial Sequence; identical to SEQ ID NO: 167
  Synthetic Construct

SEQ ID NO: 584 - RNA, 345 nt, Artificial Sequence
  Synthetic Construct
ccggauucuucuaagggugccuguggugccucccccgaagcccgcuuagggaaaaagaaaggggaagcaagcaucuccuguycagggauccccggggaaaccugaacrgaucgggcggagaagyccgugcggguccgucuucugacaggaguaaucccgygaaacaggguuuucacccuuccuuucuucggguuuccuuccucagucgaccggaccgcgucggccuucucgcgcacuucuguucggagacuacccgguggauacaacugacagaggugcuuuuucuuccacccugcuucuaccgacgcggccgggagugaagcuacccgggacccgagaggaucu

SEQ ID NO: 585 - RNA, 335 nt, Artificial Sequence
  Synthetic Construct
cggaucauuuucuugugguuccugugguacaccuccgaaggccgccuaggcgagaagagaguaaauagaggagagagcuuaccuggguucccuggcagcgcugcaacggaauycagggcacggagucggugccagaucgugcggcuucggcgccaguucgugcgagagcuucgcucgcgaacucuucguggaacggccgucgcucguggaucggccgucuuccggguuccugcucccacgcagcgauugcaaaggauccgguucgcucuccuuuuaccugcuucagaccgacgcgcccggggagugaaucaccugggaccgcgraaagcgauccu

SEQ ID NO: 586 - RNA, 227 nt, Artificial Sequence
  Synthetic Construct
cgagagugcugcggcaggaggagccgggggcggagguugucucgcucuugagagcuccgcuccuuguagcuuugagacuaccgcccuuuugcuuccuucucgcuggcugacucgaggacgcgaccggugguacaccaagaggucuccaccuccuaggguacuuuuuucuaacaccgauuccguaccgagcgccggagagugaagcgcccagggcuccaaagaagccc

SEQ ID NO: 587 - RNA, 198 nt, Artificial Sequence
  Synthetic Construct
arcgyaggcuccgarrgggyryyccarggurgarggryurgaguyyycycucaragucuggcuuagccacugaugagycyugaraurgrcgaaacuyuraraguuucgycyauyucargrcucaucaguggcuaagccagacuyugagrgagguyryyycagyyycucaccugrryrcccyyucggarcacygcaguy

SEQ ID NO: 588 - RNA, 335 nt, Artificial Sequence
  Synthetic Construct
cccauaaguuucgucgcaucccagcgacucrucagugggcuaagcccagacuuaugagagagugguuaccucucaccccuccaccuuggggugcccuauucgragcacugcaguuycgauagaaaggcuaagcacuucgcaaugaaguaaggugggacuuuucccuaggguuugagcggucgaacccagggggagugurauccagguaccgccguagaaacuggauuacgacgccuacccggrauucaaacccggucccccucagaggugacucugagugaaaggucgugcuuagcacacugaygaguuccugaaauggaacgaaaccuuucuaa

SEQ ID NO: 589 - RNA, 341 nt, Artificial Sequence; identical to SEQ ID NO: 173
  Synthetic Construct

SEQ ID NO: 590 - RNA, 326 nt, Artificial Sequence
  Synthetic Construct
ycaaaguuucgccguaucucaacggcucaucagugggcuagcccagacuuugrgagaurruaccucucagccccuccaccuuggrgugcccuauucggagcacugcaguucccgauagaaaggcuaagcaccucgcaaugaguaaggugggacuuuuccuucggaaccaagcgguugguuccgaggggggugugauccagguaccgccguagaaacuggauuacgacgucuacccgggauucaaacccgguccccuccagaagugauucuggagaagagucgugcuagcacacugaugagucucugaaaugagacgaaacucuuyu

SEQ ID NO: 591 - RNA, 324 nt, Artificial Sequence
  Synthetic Construct
gguaaacaccgugcgguuccugugguucgccccgccaacgcaragauaaagaaaacgaggagaagaaggaacucaccugucgucgucgacgaaggcggugaaaaggagcugccagcacuaagccggacggcgcccucgcaccaguuccgcuguggguucgccuacaagaacguacgguguugaggcccuguccgccgcugcgcugccaccuacucucgcgccgcuagucgagcggacuccgggugagcccccuguucucucacgcucuuuuucuuugrcgcagcggcggguggguucccaggguaaaacacaaugguguuuccc

SEQ ID NO: 592 - RNA, 322 nt, Artificial Sequence
  Synthetic Construct
ycaaaguuucgccguaucucaacggcucrucagugggcuagcccagacuuugagagaguruaccucucagccccuccaccuuggggugcccuaucggaugcaguuycgrurgaaaggcuaagaccucgcaaugagguaaggugggacuuuuccuucuggaaccaagcgguugguuccgaggggggugugayycagguaccgccguagaaacugrruuacgacgucuacccgggauucaaacccgguccccuccagaagugauucuggaugaagagucgygcuagcrcacugaygagucucugaaaugagacgaaacucuuya

SEQ ID NO: 593 - RNA, 340 nt, Artificial Sequence; identical to SEQ ID NO: 177
  Synthetic Construct

SEQ ID NO: 594 - RNA, 323 nt, Artificial Sequence
  Synthetic Construct
ycauaaguuucgccguaucucaacggcucrucagugggcuagcccagacuuaugrgagauruaccucucagcccyuccaccuurgggugcccuaycgagcacgcaguuucgrurgaaaggcuaagaycucgcaaugagruaaggugggacuuuuccuucuggaaccaagcgguugguuccgaggggggugugauccagguaccgccguagaaacuggruuacgacgucuacccgggauucaaacccgguccccuccagaaggauucuggaugaagagucugugcuagcacacugaygagucucugaaauragacgaaacucuu

SEQ ID NO: 595 - RNA, 328 nt, Artificial Sequence
  Synthetic Construct
ucaaaaguuucgucgcauccagcgacucaucagugggcuugcccagacuuuugagagaurruaccucucagccccuccaccuuggrgugcccuauucggagcacugcaguucccgauagaaaggcuaagcaccucgcaaugacguaaggugggacuuuucccuaguucragcgguygracuagaggggagugugauccugguaccgccguagaaacaggauuacgacgycuuaccgagauucaaacucggccccucucagaggugayucugagcgaaaggucgugcaagcacacugaygaguuccugaaauggaacgaaaccuuuyua

SEQ ID NO: 596 - RNA, 330 nt, Artificial Sequence
  Synthetic Construct
cccaaaguuucgucgcaucccagcgacucrucagugggcuugcccagacuuugagagaruggaccucucagccccuccaccuuggrgugcccuauucggagcacugcaguucccgauagaaaggcuaagcaccucgcaaugacguaaggugggacuuuucccuyrguuygagcggucgracycagaggggagugugauccugguaccgccguagaaacaggauuacgacgucuuaccgagauucaaacucggccccucucagaggugacucugagcgaaaggucugugcaagcacacugaygaguuccugcaauggaacgaaaccuuuca

SEQ ID NO: 597 - RNA, 335 nt, Artificial Sequence
  Synthetic Construct
accauaaguuucgccgcgucucagcggcucaucagugggcuuagcccagacuuaugagagaguaagaccucucagccccuccaccuuggrgugcccauucggagcacugcaguucccgauagaaaggcuaagcaccucgcaaugaguaaggugggacuuuccuucggraccaagcgguugguyccgaggggggugugacucagguaccgccguagaaacugaguuacgacgucuacccgggauucaaacccgguccccuccagaagugauucuggauugagagucugugcuaagcacacugaugagucucugagaugagacgaaacucucugguu

SEQ ID NO: 598 - RNA, 329 nt, Artificial Sequence
  Synthetic Construct
yycauaaguuucgccguaucucaacggcucaucagugggcuagcccagacuuaugrgagaruruaccucucagccccuccaccuuggrgugcccuauucggagcacugcaguucccgauagaaaggcuaagcaccucgcaugaguaaggugggacuuuuccuucuggaaccagcggugguuccgaggggggugugayycagguaccgccguagaaacugrruuacgacgucuacccgggauucaaacccgguccccuccagaagugauucuggaraagagucugugcuagcacacugaygagucucugaaaugagacgaaacucuuyua

SEQ ID NO: 599 - RNA, 330 nt, Artificial Sequence
  Synthetic Construct
ucaaaaguuucgucgcaucccagcgacucaucagugggcuagcccagacuuuugagagarugaugaccucucagccccuccaccuuggrgugcccuauucggagcacugcaguucccgauagaaaggcuaagcaccucgcaaugacguaaggugggacuuuucccucggguuugagcggucgaacccaaggggagugugauccugguaccgccguagaaacaggauuacgacgycuuaccgrgauucaacycggccccuucagaggugacucugaygaaaggucgygcuagcrcacugaygaguuccugaaauggaacgaaaccuuucyc

SEQ ID NO: 600 - RNA, 332 nt, Artificial Sequence
  Synthetic Construct
cycaaaguuucgucgcaucccagcgacucrucagugggcuugcccagacuuugagagaaurguyaccucucagccccuccaccuuggrgugcccuauucggagcacugcaguucccgauagaaaggcuaagcaccucgcaaugacguaaggugggacuuuucccuacaguucgagcggucgaacuagaggggaguguaaccuugguaccgccguagaaacaggguuacgacgucuuaccgagauuccacucggccccucucagaggugacucugagygaaaggucgugcaagcacacugaygaguuccugaaauggaacgaaaccuuuyura

SEQ ID NO: 601 - RNA, 333 nt, Artificial Sequence
  Synthetic Construct
yycauaaguuucgycgyaucycarcgrcucaucagugggcuagcccagacuuaugagagagugruaccucucagccccuccaccuuggggugcccuauucgaggcacugcagucucgauagaaaggcuaagraccucgcaaugagguaaggugggacuuuuccuucuggaaccagcgguugguuccgaggggggugugauccagguaccgccguagaaacuggauuacgacgucuacccgggauucaaacccgguccccuccagaagugauucuggaugaagagucgugcuuagcacacugaugagucucugaaaugagacgaaacucuuyua

SEQ ID NO: 602 - RNA, 329 nt, Artificial Sequence
  Synthetic Construct
yccauaaguuucgccguaucucaacggcucgucagugggcuuagcccagacuuaugagagagugruaccucucarcccyuccaccuurgrgugcccuauucggagcaugcaguucccgauagaaaggcuaagcaccucgcraugagguaaggugggacuuuuccuucggaaccagcggugguuccgaggggggugurayycagguaccgccguagaaacugrruuacgacgucuacccgggauucaacccgguccccuccagaagugauucuggagaagagucgugcuaagcacacugacgagucucugaaaugagacgaaacucuucu

SEQ ID NO: 603 - RNA, 318 nt, Artificial Sequence
  Synthetic Construct
cccauaaguuucgucgcaucccagcgacucrucagugggcuaagcccagacuuaugagagagugguuaccucucagccccuccaccuuggggugcccuauucggagcacugcaguuycgauagaaaggcuaagacyucgcaaugarguaaggugggacuuuuccyugggyyragcgguygryccccagguaccgccguagaaacuggauuacgacgycuacccgggauucaaacccggucccccucagaggugacucugagugaaaggucgugcuuagcacacugaygaguuccugaaauggaacgaaaccuuuyuaa

SEQ ID NO: 604 - RNA, 336 nt, Artificial Sequence
  Synthetic Construct
acucaucagugggcuaagcccagacuuaugagagagugguuaccucucagccccuccaccuuggggugcccuauucggagcacugcaguucccgauagaaaggcuaagcacuucgcaaugaaguaaggugggacuuuucccuaggguuugagcggucgaacccagggggagugugauccagguaccgccguagaaacuggauuacgacgccuacccgggauucaaacccggucccccucagaggugacucugagugaaaggucgugcuuagcacacugaygaguuccugaaauggaacgaaaccuuuyuaacyauaaguuucgucgcaucccagcg

SEQ ID NO: 605 - RNA, 326 nt, Artificial Sequence
  Synthetic Construct
ycaaaguuucgccguaucucaacggcucaucagugggcuaagcccagacuuugrgagagugaugaccucucagccccuccaccuuggggugcccuaucgrcacugcagyyycgaurgaaaggcuaagaccucgcaaugagguaaggugggacuuuuccuucggaaccaagcgguugguuccgaggggggugugauycagguaccgccguagaaacuggauuacgacgucuacccgggauucaaacccgguccccuccagaagugauucuggagaagagucgugcuuagcacacugaugagucucugaaaugagacgaaacucuuyy

SEQ ID NO: 606 - RNA, 338 nt, Artificial Sequence
  Synthetic Construct
aucaaaaguuucgccguaucucaacggcucaucagugggcuuagcccagacuuuugrgagaauaaayaccucucagccccuccaccuuggggugcccuauucggagcacugcaguucccgauagaaaggcuaagcaccucgcaaugagguaaggugggacuguuccuucuggagccaagcgguuggcuccgaggggggugugauccagguaccgccguagaaacuggauuacgacgucuacccgggauucaaacccgguccccuccagaagugauucuggauygaagagucgugcuaagcacacugaugagucucugagaugagacgaaacucuucyc

SEQ ID NO: 607 - RNA, 335 nt, Artificial Sequence
  Synthetic Construct
rucaaaaguuucgucacauuucagugacucaucagugggcuuagcccaracuuuugrgagauaaaaccucucagccccuccaccuuggggugcccuauuggagcacugcaguucccgauagaaaggcuaagcaccucgcaaugagguaagguggracuuuuccuucugagaccaagcgguuggucucgaggggggugugacycagguaccgccguagaaacugrguuacgacgucuacccgggauucaaacccgguccccuccagaagugaauucuggaugaagagucugugcuaagcacacugaygagucccugagaugggacgaaacucuucy

SEQ ID NO: 608 - RNA, 325 nt, Artificial Sequence
  Synthetic Construct
yycauaaguuucgccguaucucaacggcucaucagugggcuagcccagacuuaugrgagagurrugaccucucagcccyuccaccuurgrgugcccuauucggagcacugcaguucccgauagaaaggcuaagcaccucgcaugagguaaggugggacuuuuccuucggarccaagcgguuggyuccgaggggggugugayycagguaccgccguagaaacugrruuacgacguyuacccggrauucaacccgguccccuccagaagugauucuggagaagagucuagcrcacugaugagucucugaaagagacgaaacucuuuc

SEQ ID NO: 609 - RNA, 339 nt, Artificial Sequence
  Synthetic Construct
caaauuaguuucgccguaucucaacggcucaucagugggcuuugcccagacuaauuugagaguagucaccucucagcccuuccaccuuagrgugcccuauucggagcacugcaguucccgauagaaaggcuaagcaccucgcaaugagguaaggugggacuuuuccuucuggaaccaagcgguugguuccgaggggggugugacccagguaccgccguagaaacuggguuacgacgucuacccgggauucaaacccgguccccuccagaagugauucuggaugaagagucugcgcaaagcgcacugaugagucucugaaaugagacgaaacucuuuuga

SEQ ID NO: 610 - RNA, 339 nt, Artificial Sequence; identical to SEQ ID NO: 194
  Synthetic Construct

SEQ ID NO: 611 - RNA, 334 nt, Artificial Sequence
  Synthetic Construct
ucaaaaguuuygccgcauuucagcggcucaucagugggcuuagcccagacuuuugagagaguarygaccucucagccccuccaccuuggggugcccuauucggagcacugcaguuccgauagaaaggcuaagaccucgcaaugarguaaggugggacuuuuccuucggaaccaagcgguugguuccgaggggggugugauccagguaccgccguagaaacuggauuacgacgucuacccgggauucaaacccgguccccuccagaagugauucuggaagaugagucugugcuaagcacacugaugagucucugaaaugagacgaaacucauauc

SEQ ID NO: 612 - RNA, 339 nt, Artificial Sequence; identical to SEQ ID NO: 196
  Synthetic Construct

SEQ ID NO: 613 - RNA, 338 nt, Artificial Sequence; identical to SEQ ID NO: 197
  Synthetic Construct

SEQ ID NO: 614 - RNA, 332 nt, Artificial Sequence
  Synthetic Construct
cycauaaguuucgccguaucucaacggcucrucagugggcuaagccyagacuuaugagagagurguyaccucucagccccuccaccuuggggugcccuaucggacacugcaguuycgaurgaaaggcuaagacgucgcaaugacguaaggugggacuuuuccuucuggaaccagcgguugguuccgaggggggugugauccagguaccgccguagaaacugrruuacgacgucuacccgggauucaaacccgguccccuccagaagugauucuggaugaagagucgugcuuagcacacugaygagucucugaaaugagacgaaacucuuyur

SEQ ID NO: 615 - RNA, 332 nt, Artificial Sequence
  Synthetic Construct
accauaaguuucgccguaucucaacggcucaucagugggcuuagcccagacuuaugrgaguuagucaccucucagccccuccaccuuggggugcccuauccgargcacgcaguuucgguagaaaggcuaagcaucucgcaaugagauaaggugggacuuuuccuucuggaaccaagcgguugguuccgagggggggugauccagguaccgccguagaaacuggauuacgacgucuacccgggauucaaacccgguccccuccagaaggauuyuggaugaagagyygugcuaagcacacugaugagucuyugaaaugagacgaaacucuuuug

SEQ ID NO: 616 - RNA, 312 nt, Artificial Sequence
  Synthetic Construct
yccaaaguuucgucgcaucccagcgacucrucagugggcuagcccagacuuugrgagarugruyaccucucagccccuccaccuuggggugcccuauycgagcacugcgyuucgruagaaaggcuaagcacucgcaaugaguaaggugggacuuuucccugrguuygagcggucgracycagggggagugurauccgguaccgccguagaaacuggauuacgacgycuacycggycccycucagaggugacucugagygaaaggucgugcuagcacacugaygaguuccugaauggaacgaaaccuuucura

SEQ ID NO: 617 - RNA, 326 nt, Artificial Sequence
  Synthetic Construct
cycaaaaguuucgucgcaucccagcgacucaucagugggcuugcccagacuuuugagagaaugyaccucucagccccuccaccuuggggugcccuauccgagcacugcugyuucgguagaaaggcuaagacgucgcaaugacguaaggugggacuuuucccuygaguuyragcgguygracucagaggggagugugauccgguaccgccguagaaacuggauuacgacgucuacccgggauucaaacucggccccucucagaggugaucugrgygaaaggucgygcaagcrcrcugaugaguuccugaauggaacgaaaccuuuyr

SEQ ID NO: 618 - RNA, 338 nt, Artificial Sequence; identical to SEQ ID NO: 202
  Synthetic Construct

SEQ ID NO: 619 - RNA, 327 nt, Artificial Sequence
  Synthetic Construct
ycauaaguuucgccguaucucaacggcucrucagugggcuagcccagacuuaugrgagaruruaccucucagccccuccrccuuggrgugcccuauucgaggcacugcagucucgauagaaaggcuaagacgucgyraugacguaaggugggacuuuuccuucggaaccagcgguugguuccgaggggggugugauccagguaccgccguagaaacuggauuacgacgucuacccgggauucraacccgguccccuccagaagugauucuggaraagagucgygcuagcrcacugaugagucucugaaaugagacgaaacucuuuua

SEQ ID NO: 620 - RNA, 290 nt, Artificial Sequence
  Synthetic Construct
caccccuccaccuuggggugcccuauucggacacugcaguuccgauagaaaggcuaagaccucgcaaugagguaaggugggacuuuuccuucggaaccaagcgguugguuccgagggggugugauccagguaccgccguagaaacuggauuacgacgucuacccgggauucaaacccgguccccuccagaagugauucuggagagagucugugcuagcacacugaugagucucugaaaugagacgaaacucuuaycaaaguuucgccgyauyucarcggcucaucagugg

SEQ ID NO: 621 - RNA, 338 nt, Artificial Sequence; identical to SEQ ID NO: 205
  Synthetic Construct

SEQ ID NO: 622 - RNA, 328 nt, Artificial Sequence
  Synthetic Construct
acucauaaguuucgucrcaucycagygacucrucagugggcuagcccagacuuaugrgagaurraccucucagccccuccaccuuggggugcccuauucggagcacugcaguucccgauagaaaggcuaagcacgucgcuugacguaaggugggacuuucccuaggguuugagcggucgaacccagggggagugugacccagguaccgccguagaaacuggguuacgacgccuacccgggauucaaacccggucccccucagaggugacucugagugaaaggucgcgcuaagcgcacugaugaguuccugaaauggaacgaaaccuuu

SEQ ID NO: 623 - RNA, 326 nt, Artificial Sequence
  Synthetic Construct
cauaaguuucgccuuruuucaaaggcucaucagugggcuuagcccagacuuaugagaguaaraccucucagcccuuccaccuurgggugcccuauucggrrcacugcaguuccgauagaaaggcuaagcaccucgcaaugrgguaaggugggacuuuuccuucuggaaccaagcgguugguuccgaggggggugugauccagguaccgccguagaaacuggauuacgacgucuacccgggauucaaacccgguccccuccagaagugauucuggaugaagagucgygcuaagcrcacugaygagucucugaaaygagacgaaacuc

SEQ ID NO: 624 - RNA, 330 nt, Artificial Sequence
  Synthetic Construct
cucauaaguuycgccguaucucaacggcucaucaguggcuaagcccagacuuaugggagaguagucaccucucagccccuccaccuuggggugcccuaucggagcacucaguuccgauagaaaggcuaagaccucgcaaugagguaaggugggacuuuuccuucuggaaccuagcgguagguuccgagggggguggauccagguaccgcyguagaaacuggauuacgacgucuaccgagauucaaacccgyccccccagaagugauucuggaugaagagucugugcuuagcacacugaugagucucugaaaugagacgaaacucuucuga

SEQ ID NO: 625 - RNA, 321 nt, Artificial Sequence
  Synthetic Construct
ycauaaguuucgccguauccaacggcucaucaguggcuaagcccagacuuaugrgagagugaugaccucucagccccuccaccuuggrgugccuauucgagccugcaguucccgauagaaaggcuaagaccucgcaaugagguaaggugggacuuuuccuucggaaccagcggugguuccgaggggggugugauccaggurccgccguagaaacuggauuacgacgucuacccgggauucaaacccguccccuccagaaguguycuggagaagagucgygcuuagcryacugaugagucucugaaaugagacgaaacucuy

SEQ ID NO: 626 - RNA, 337 nt, Artificial Sequence; identical to SEQ ID NO: 210
  Synthetic Construct

SEQ ID NO: 627 - RNA, 332 nt, Artificial Sequence
  Synthetic Construct
ucaaaaguuucgucgcauccagcgacucaucagugggcuuugcccagacuuuugagagarugauaccucucagccccuccaccuuggggugcccuauccgagcacugcugyuucgguagaaaggcuaagcacgucgcaaugacguaaggugggacuuuucccuyaguucaagcgguugracuyagaggggagugugauccugguaccgccguagaaacuggauuacgacgucuacccgggauucaaacucggccccucucagaggugacucugagcgaaaggucugugcaaagcacacugaygaguuccugaaauggaacgaaaccuuucua

SEQ ID NO: 628 - RNA, 333 nt, Artificial Sequence
  Synthetic Construct
ucaaaaguuucgucgcaucccagcgacucaucagugggcuuagcccagacuuuugagagaauagucaccucucagccccuccaccuuggggugcccuauccgarcacugcuguuucgguagaaaggcuaagcacgucgcaaugacguaaggugggacuuuucccuacaguucgagcggucgaacuagaggggagugugauccugguaccgccguagaaacuggauuacgacgucuacccgggauucaaacucggccccucucagaggugacucugagcgaaaggucgugcuaagcacacugaugaguuccugaaauggaacgaaaccuuuyua

SEQ ID NO: 629 - RNA, 337 nt, Artificial Sequence; identical to SEQ ID NO: 213
  Synthetic Construct

SEQ ID NO: 630 - RNA, 337 nt, Artificial Sequence; identical to SEQ ID NO: 214
  Synthetic Construct

SEQ ID NO: 631 - RNA, 331 nt, Artificial Sequence
  Synthetic Construct
ycaaaguuucgucgcaucycagcgacucaucagugggcuagcccagacuuaugagagagugaugaccucucagccccuccaccuuggggugcccuayygragcacugcaguuycgrurgaaaggcuaagacgucgcaaugacguaaggugggacuuuucccuaggguuugagcggucgaacccagggggagugugayccagguaccgccguagaaacuggruuacgacgycuacccgggauucaaacccggucccccucagaggugacucugagugaaaggucgcgcuuagcgcacugaugaguuccugcaauggaacgaaaccuuucuca

SEQ ID NO: 632 - RNA, 330 nt, Artificial Sequence
  Synthetic Construct
ucauaaguuucgccguaucucaacggcucrucagugggcuaagcccagacuuaugagagruagucaccucucagccccuccaccuuggggugcccuaucgagcacugcagucucgaurgaaggcuaagacgucgcaaugacguaaggugggacuuuuccuucuggaaccaagcgguugguuccgaggggggugugauccagguaccgccguagaaacuggauuacgacgucuacccgggauucaacccgguccccuccagaagugauucuggaugaagagucgugcuuagcacacugaygagucucugaaaugagacgaaacucuuuuga

SEQ ID NO: 633 - RNA, 334 nt, Artificial Sequence
  Synthetic Construct
ucaaaaguuucgccgcauuucagcggcucaucagugggcuuagcccagacuuuugagagaguaaugaccucucagccccuccaccuuggggugcccuauucggagcacugcaguucccgauagaaaggcuaagcacucgcaaugaguaaggugggacuuuucccuaggguuugagcggucgaacccagggggagugurauccagguaccgccguagaaacuggauuacgacgycuacccgggauucaaacccgguccccuccagaagugauucuggaagaugagucugugcuaagcacacugaugagucucugaaaugagacgaaacucauauc

SEQ ID NO: 634 - RNA, 337 nt, Artificial Sequence
  Synthetic Construct
cuuuugagagaguaaugaccucucagccccuccaccuuggggugcccuauucggagcacugcaguucccgauagaaaggcuaagcacuucgcaaugaaguaaggugggacuuuucccuaggguuugagcggucgaacccagggggagugugauccagguaccgccguagaaacuggauuacgacgycuacccgggauucaaacccgguccccuccagaagugauucuggaagaugagucugugcuaagcacacugaugagucucugaaaugagacgaaacucauaucaucaaaaguuucgccgcauuucagcggcucaucagugggcuuagcccaga

SEQ ID NO: 635 - RNA, 337 nt, Artificial Sequence; identical to SEQ ID NO: 219
  Synthetic Construct

SEQ ID NO: 636 - RNA, 333 nt, Artificial Sequence
  Synthetic Construct
aacauaaguuucgccuurucgcaaaggcucaucagugggcuuagcccagacuuaugugagaguuaayaccucucagcccuuccaccuuagggugcccuauucggacacugcaguuycgauagaaaggcuaagcaccucgcaaugagguaaggugggacuuuuccuucggraccaagcgguugguuccgaggggggugugauccagguaccgccguagaaacugrruuacgacgucuacccggrauucaaacccgguccccuccagaagugauucuggagaagagucgcgcuaagcgcacugaugagucuuugarauaagacgaaacucuucua

SEQ ID NO: 637 - RNA, 335 nt, Artificial Sequence
  Synthetic Construct
aucaaaaguuucgccguaucucaacggcucaucaguggcuaagcccagacuuuugagagagugaugaccucucagcccuuccaccuuggggugcccuauucggagcacugcaguucccgauagaaaggcuaagcacgucgcaaugacguaaggugggacuuuuccuucuggaaccaagcgguugguuccgaggggggugugacucagguaccgccguagaaacugaguuacgacgucuacccggaauucaacccguccccuccagaagygauucuggaagauaagucugugcuuagcacacugaugagucucugaaaugagacgaaacuuaucuc

SEQ ID NO: 638 - RNA, 336 nt, Artificial Sequence; identical to SEQ ID NO: 222
  Synthetic Construct

SEQ ID NO: 639 - RNA, 336 nt, Artificial Sequence; identical to SEQ ID NO: 223
  Synthetic Construct

SEQ ID NO: 640 - RNA, 335 nt, Artificial Sequence
  Synthetic Construct
cccaacuuaugagagagugguuaccucucagccccuccaccuuggggugcccuauucgragcacugcaguuycgauagaaaggcuaagcacuucgcaaugaaguaaggugggacuuuucccuaggguuugagcggucgaacccagggggagugugauccagguaccgccguagaaacuggauuacgacgccuacccgggauucaaacccggucccccucagaggugacucugagugaaaggucgugcuuagcacacugaygaguuccugaaauggaacgaaaccuuucuaacyyauaaguuucgucgcaucccagcgacucrucagugggcuaag

SEQ ID NO: 641 - RNA, 336 nt, Artificial Sequence; identical to SEQ ID NO: 225
  Synthetic Construct

SEQ ID NO: 642 - RNA, 313 nt, Artificial Sequence
  Synthetic Construct
gguaaacaccgugcgguuccugugguucgccccgccaacgcagauagauaaagaaaacgaggagaagaaggaacucaccugucgucgucgacgaaggccggugagaaaggagcugcagcacuaacgacggcgyugcryguuccgcugugyyraagaacguacgguguugaggcccuguccgccgcugcgcugccaccuacucucgcgccgcuagucgagcggacuccggguggagcccccuguucucucacgcucuuuuucuuugacgcagcggcggguggguucccaggguaaaacacaauagguguuuccc

SEQ ID NO: 643 - RNA, 327 nt, Artificial Sequence
  Synthetic Construct
ggguggugugugccaccccugaugagaccraaaggucgaaaugggguuucgccaugggucggracuuuaaauucggaggauucguccuuaacguuccuccaagaguyccuuccccaaacccuuacuuguaagugguucggcgaaugaccguuucguccuuuyggacucaucagggaaaguacacacuuuccgacggugrguucgucgacaccucucccccycccagguacuaucccuuuucaaggauguguucccuaggrggguggguguaccucuuuuggauugcuccggycuuccaggagagauagaggacgaccucuccccaua

SEQ ID NO: 644 - RNA, 319 nt, Artificial Sequence
  Synthetic Construct
ggguggugugugccaccccugaugagaccgaaaggucgaaaugggguyuygccaugggugggcuuuaaauucggaggaucguccuuaacguuccuccaagrgcccuccccaacccuuacuuguaagggucggcgaruguaccguuucguccuuucggacucaucagggaaagyacacacuuuccgacggugrgucguygacaccucucccccucccagguacuaucccyuuucaaggauguguucccuaggagggugggyguaccucuuuuggauugcuccggycuuccaggagagauagaggacgaccucuccccaua

SEQ ID NO: 645 - RNA, 328 nt, Artificial Sequence
  Synthetic Construct
ggguggugugugccaycccugaugagaccaaaaggucgaaaugggguuucgccaugggucuggucuuuaaauucggaggaucguccuuuaacguuccuccaagagaccauuccccaaacccuuacuuyguaagugguucggcgaauguaccguuucgcucuuuugagcucaucagggacaguacacacuguccgacgguagagucgucgacaccucucccccccccagguacuauccucuuucaaggauruguucccyagggggguggguguaccucuuuuggauucuccggycuuccaggagagauagaggacgaccucuccccaua

SEQ ID NO: 646 - RNA, 334 nt, Artificial Sequence
  Synthetic Construct
ggguggugugugccaccccugaugaguccgaaaggacgaaauggggucucuccaugggucggaaccuuaaauucggaggauucguccuuuaaacguuccuccaaggguuccuuccccgaucccuuacuuucguaaguauggaacggagaguguaccguuucgcccuuuugggcucaucagggaaagcgcacacuuuccgacggugrguyguygacaccucucccccucccagguacuaucccuuuucaaggauguguucccuaggaggguggguguaccucuuucuggauuucuccggucuuccaggagagguagaggacgaccucuccccaua

SEQ ID NO: 647 - RNA, 333 nt, Artificial Sequence
  Synthetic Construct
gggugguguguaccacyccugaugaguccraaaggacgaaaugggguucugccaugggguagagcuuuaaauucggaggauucgaccuuaaaucguuccuccaagagcucuucccccaucccuuacuurguaaguacggaucggcggauguaccguuucgcccuuucgggcucaucagggaaagcgcacacuuuccgacggugaguucgucgacaccucugccccucccagguacuauccyyuuucaaggauguguucccuaggagggugaguguaccucuuuucggauuucuccggucuuccgagagagacagaggacggccucuccccaua

SEQ ID NO: 648 - RNA, 335 nt, Artificial Sequence; identical to SEQ ID NO: 232
  Synthetic Construct

SEQ ID NO: 649 - RNA, 319 nt, Artificial Sequence
  Synthetic Construct
ccggugagaaaggagcugccagcacuaagccggacggcgcccucgcaccagucccugugggyuygccycaagacgcgguguugaggcccuguccgccgcugcrcugccaccucucuucgcgccgcuagucgagcggacycrggyggyuucccuauucucucacgcucuuuucuuuuugygcagcggcggguggguucccaggguaaaacacaauugguguuucccgguaacaccgugcgguuccugugguucgccccgccaaagcauaauuaaagaaaacgaggagaagaaggaacucaccugucgucgucgacgaagg

SEQ ID NO: 650 - RNA, 320 nt, Artificial Sequence
  Synthetic Construct
uaaacaccgugcgguyccugugguucgccycgccaagcaaauuaaagaaaacgaggagaagaaggaacucaccugucgucgucgacgaaggccggugagaaaggagcugycagcacuaagccggacggcgcccucgcaccaguuccgcugyggguucgccurcaagaacgyaygguguugaggcccuguccgccgcugcgcugccaccuacucuucgcgccgcuagucgagcggacuccrgguggagycccuruucucucacgcucuuuuyyuuugcgcagcggcgrguggguucccaggguaaaacacaaugguguuuc

SEQ ID NO: 651 - RNA, 334 nt, Artificial Sequence; identical to SEQ ID NO: 235
  Synthetic Construct

SEQ ID NO: 652 - RNA, 306 nt, Artificial Sequence
  Synthetic Construct
agacuucuugugguuccuguggugcaccccucagcccuaccugcgaaagaaaaaagrguuagaaggcggcagaggagcugacuggucgucgucgacgaaggcucgucagcugcggagguuggggucgacuggccuccgguggcgaagyugagcucucucuucuagcuguaaccggaccgguccccuucacccgagcgcugcuugccgcuagucgagcggacuuccaagucccucccrgccgcuuuucuuucccurauucyguagcagcggggagagggugaagccccugaaccccugagggcuccu

SEQ ID NO: 653 - RNA, 319 nt, Artificial Sequence
  Synthetic Construct
ggguggugugugccaccccugagagaccgaaaggucgaaaugggguuucccaugggggracuuuaaauucggaggauucguccyuuaaacguuccuccaagrguycyyuccccaacccuuacuuguaagyggucggcgarugaccguuucguccuuucggacucaucagggaaguacacacuuccgacggyrrrucgucgacaccucucccccycccagguacuauccyuuuycaargauguguucccuagggggguggguguaccucuuuuggauucuccggycuuccaggagagauagaggacgaccucuccccaua

SEQ ID NO: 654 - RNA, 332 nt, Artificial Sequence
  Synthetic Construct
ggguggugugugccaccccugaugagaccgaaaggucgaaaugggguuucgcyauggguggraccuuaaauucgggggauucguccuuyaaacguucccccaaggguyccuuccccaaucccuuacuuuguaaguacggaucggcgaauguaccguuucguccuuucggacucaucagggaaaguacacacuuuccgacggcgrguucgucgacaccucucccccycccagguacuaucccuuuucaaggauguguucccuaggrggguggguguaccucuuuuggauugcuccggycuuccaggagagauagaggacgaccucuccccaua

SEQ ID NO: 655 - RNA, 334 nt, Artificial Sequence; identical to SEQ ID NO: 239
  Synthetic Construct

SEQ ID NO: 656 - RNA, 334 nt, Artificial Sequence; identical to SEQ ID NO: 240
  Synthetic Construct

SEQ ID NO: 657 - RNA, 334 nt, Artificial Sequence; identical to SEQ ID NO: 241
  Synthetic Construct

SEQ ID NO: 658 - RNA, 332 nt, Artificial Sequence
  Synthetic Construct
guguacugacugcagccaugcaagaaaaagaacgggagaagaagcguaagagcggucucaggagccccggggcaacucaaaccgagcggggucuugaccaguggcgagcgcccuguucaaacaggaguaaucccagcugaaacaggguuuucacccuuccuuucuucugguuuccuuccucugcuucagcggccucgcccggagucuugaccagcgcagguucugacgcgaccgguggcaucaccgaguuucgcucaaaccucaaucuccuuuuucuucauucuacuugggucuccgggcaaggggguuuaccccuugaaaccgcauuugga

SEQ ID NO: 659 - RNA, 315 nt, Artificial Sequence
  Synthetic Construct
ccggugagaaaggagcugccagcacuaagccggacggcgcccucgcaccaguuccgcuguggguucgccuacaagaacguacgguguugaggcccuguccgccgcugcgcugccaccuacucucgcgccgcuagucgagcggacucrggyggrryycccurcucucacgcucuuuuucuuugcgcagcggcggguggguucccaggguaaaacacaaugguguuucccgguaaacaccgugcgguuccugugguucgccccgccaagcaaaugauaaagaaaacgaggagaaraaggaacucaccugucgucguc

SEQ ID NO: 660 - RNA, 316 nt, Artificial Sequence
  Synthetic Construct
ggaggaaacuccgugugguuccugurggucaccccccggycccucuaagaaaagaaaggyaaggagacucaccugucgucgucgacgaaggcaugugagcyacucrugaagagcgucagcggacggccucygagacgagcgauggacaguagagcucgycucuaccagucgcgugcugacucucrcgcccacccgcacggcugcuccgagrgrrggcucucugccgcuagucgagcggaccagagugacucyccyrcccuauuuucrcrgagrccggcgguggagccycaggguaaaacacgauugguguuucccc

SEQ ID NO: 661 - RNA, 324 nt, Artificial Sequence
  Synthetic Construct
ggguggugugurccaycccugaugagaccraaaggucgaaaugggguyucgccaugggcgrcuaaauucggaggauucguccuuaacguuccuccaaggguccucuccccaaucccuuacuuruaaguuggaucggcgaguguaycguuucguccuuucggacucaucagggacaguacacacuguccgacggcagagucgucgacaccucucccccccccagguacuaucccuuuucaaggauguguucccuagggggguggguguaccucuuuuggauugcuccggucuuccaggagagauagaggacgaccucuccccaua

SEQ ID NO: 662 - RNA, 322 nt, Artificial Sequence
  Synthetic Construct
ggguggugugugccaccccugaugagaccgaaaggucgaaaugggguuyugccaugggggacyuuaaauucggrrgrucguccyuyaacguuccuccaaggguccuuuccccaaucccuuacuuguaagacggaucggcggauguaccguuucguccuuucggacucaucagggacaguacacacuuccgacggurrrucgucgacaccucucccccycccagguacuauccyuuuycaaggauuguucccuaggrggguggguguaccucuuuuggauucuccggycuuccaggagagauagaggacgaccucuccccaua

SEQ ID NO: 663 - RNA, 333 nt, Artificial Sequence; identical to SEQ ID NO: 247
  Synthetic Construct

SEQ ID NO: 664 - RNA, 179 nt, Artificial Sequence
  Synthetic Construct
ugccagcacuaagccggacggcgcccucgcaccaguuccgcuguggguucgccuacaagaacguacgguguugaggcccuguccgccgcugcgcugccaccuacucuucgcgccgcuagucgagcggacuccggguguagcccccuguucucucacgcucuuuuucuuugacgcagcgg

SEQ ID NO: 665 - RNA, 322 nt, Artificial Sequence
  Synthetic Construct
gguaaacaccgugcgguuccuaurgcgacccgccaacgcagauagauaaagaaaacgggaagaaggaacucaccuucgucgucgacgaaggccggugagaaaggagcugccagcacuaagccggacggcgcccucgcaccaguuccgcugyggguucgccurcaagaacgyaygguguugaggcccuguccgccgcugcgcugccaccuacucucgcgccgcuagucgagcggacuccggguggagcccccuguucucucacgcucuuuuucuuugacggcggcggguggguucgcaggguaaaacacaauagguguuuccc

SEQ ID NO: 666 - RNA, 195 nt, Artificial Sequence
  Synthetic Construct
ugaarrggagcurycgcryurgcggrcgrccgaccyrygguyrccraaracayguguugrgrccyryccgccgcugcgyyrcaccyrycgccrcuagyggcgrayrggugyrrcgcagcggcggryrggcycaacaccgurcguucyugurgyrcccrcarcgarurgragrrrcgagrrgarcucyyuucrycg

SEQ ID NO: 667 - RNA, 331 nt, Artificial Sequence; identical to SEQ ID NO: 251
  Synthetic Construct

SEQ ID NO: 668 - RNA, 255 nt, Artificial Sequence
  Synthetic Construct
aauucrccuucucgucgacgaaggccggugagaaaggagcugccagcacuaagccggacggcgcccucgcaccaguuccgcuguggguucgccuacaagaacguacgguguugaggcccuguccgccgcugcgcugccaccuacucuucgcgccgcuagucgacggacuccggguguagcccccuguucucucacgcucuuuuucuuugacgcacggcggguggguucccaggguaaacayaauagguguuuccc

SEQ ID NO: 669 - RNA, 325 nt, Artificial Sequence
  Synthetic Construct
gucgacgaaggccggugagaaaggagcugccagcacuaagccggacggcgcccucgcacyaguuccgcuguggguucgccuacaagaacguacgguguugaggcccuguccgcugcugcgcugccaccuacucuugcgccgcuagucgagcrgacuccggguggagcccyyugucucccucgcucuuuuucuuugacgcagcggguggguucccaggguaaaacacaauagguguuucccgguaaacrccgugcgguuycugugguucgccccgccaaccagauagauaaagaaaacgaggagaagaagraacucaccugucguc

SEQ ID NO: 670 - RNA, 331 nt, Artificial Sequence; identical to SEQ ID NO: 254
  Synthetic Construct

SEQ ID NO: 671 - RNA, 330 nt, Artificial Sequence; identical to SEQ ID NO: 255
  Synthetic Construct

SEQ ID NO: 672 - RNA, 318 nt, Artificial Sequence
  Synthetic Construct
gguaaacaccgugcgguuccuggyucgccacccaacagcuacuauagaaaacgaauugaagaaugaauucccaugucgucgucgacgaaggccggugagaaaggagcugccagcacuaagccggacggcgcccucgcaccaguuccgcuguggguucgccuacaagaacguacgguguugaggcccuguccgccgcugcgcugccaccuacucucgccuagucgagcggacuccggguggagcccccuguucucucacgcucuuuuucuuugacgcagcggcggguggguucccaggaaaacacaauagguguuuccc

SEQ ID NO: 673 - RNA, 305 nt, Artificial Sequence
  Synthetic Construct
caccgugcgguuccugugguucgccgccaacgcagauagauaarraacgaggagaagaaggaacucaccugucgucgucgacgaaggccggugagaaaggagcugccagcacrrgccggacggcgcccucgcaccaguuccgyyguggucgccuaagaacguacgguguugaggcccuguccgccgcugcgcugccaccuacucuccgcgcuagucgagcggacuccggguggagcccccugcucacgcuuucuuugacgcagcggcggguggguucccaggguaaaacacaauagguguuuccc

SEQ ID NO: 674 - RNA, 325 nt, Artificial Sequence
  Synthetic Construct
cggagacuucuugugguuccuguggugacaccccucagcccuaccugcgaaagaaaaagacauuagaggcuccagaggagacugagcggucgucgucgacgaaggcuccucagucgcagagcgccgcuggaucgacuggccuccgguggaaagaagaagcuuugucuucaauuucuguaaccggaccggucuccuucggccgccgagcgcugguagccgcuagucgagcggacuuccgacucuucccucccgaggagcuuucuuyuugucygaauuccguagcagcggggagagggugaagcccugugaaccccugagggcuccu

SEQ ID NO: 675 - RNA, 329 nt, Artificial Sequence; identical to SEQ ID NO: 259
  Synthetic Construct

SEQ ID NO: 676 - RNA, 317 nt, Artificial Sequence
  Synthetic Construct
ggucccaaccaguuuucuuuayccuauuuuucccuggggcgcccggucgugguuacccuggagcucccuguuuggaggcccugggcaccaacuagagguuccugugguacucaccgaaggccgcgaagcuaggaaagaaaaagauagaaaagcuggguaagacucaccuggcgacucgucgucgacgaaggguccucagcagagcaccggcaggaggcgcuaugcaggaacgcugggguccuccagcggaggacugaagaaacuccgguuucuucuuucacucugagcuggaaucccuguggcuuguggracygggg

SEQ ID NO: 677 - RNA, 329 nt, Artificial Sequence; identical to SEQ ID NO: 261
  Synthetic Construct

SEQ ID NO: 678 - RNA, 329 nt, Artificial Sequence; identical to SEQ ID NO: 262
  Synthetic Construct

SEQ ID NO: 679 - RNA, 329 nt, Artificial Sequence; identical to SEQ ID NO: 263
  Synthetic Construct

SEQ ID NO: 680 - RNA, 319 nt, Artificial Sequence
  Synthetic Construct
agacuucuugugguuccuguggugacaccccucagcccurccugcgaaagaaaaaagucauyagaaggcgccagaggagacugagcggucgucgucgacgaaggcuccucagucgcagagcgccgcuggaucgacuggccuccgguggaaaacgaagauucgucuucaauuucuguaaccggaccggucuccuucggccgccgagcgcuggugccgcuagucgagcggacuuccgucucucccucccgaggcgcuuuucuyugaccgacuuccguagcagcggggagaggggaagcccygugaaccccugagggcuccu

SEQ ID NO: 681 - RNA, 325 nt, Artificial Sequence
  Synthetic Construct
gguaaacaccgugcgguuccugugguucgccccgccaacgcagauagauaaagaaaacgaggagaagaaggaacucaccugucgucgucgacgaaggccggugagaaggagcugccagcacuaagccggacggcgcccucgcaccaguuccgcuguggguucrccurcaagaacguacgguguugaggcccuguccgcugccgcugccaccuacucuucgcgccgcuagucgagcggacuccggguggagcccccuguucucccacgcucuuuuucuuugacrgcggcggggrguucccrgrguaaaacacaauagguguuuccc

SEQ ID NO: 682 - RNA, 329 nt, Artificial Sequence; identical to SEQ ID NO: 266
  Synthetic Construct

SEQ ID NO: 683 - RNA, 329 nt, Artificial Sequence; identical to SEQ ID NO: 267
  Synthetic Construct

SEQ ID NO: 684 - RNA, 329 nt, Artificial Sequence; identical to SEQ ID NO: 268
  Synthetic Construct

SEQ ID NO: 685 - RNA, 328 nt, Artificial Sequence; identical to SEQ ID NO: 269
  Synthetic Construct

SEQ ID NO: 686 - RNA, 323 nt, Artificial Sequence
  Synthetic Construct
ggcucgucagcugcggagguuggggucgacuggccuccgguggcgaagcugaguugagcuucgcucuucucuucuuagcuguaaccggaccgguccccuucacccggcgcugcuugccgcuagucgcgcggacuucaagucucccucccgagccgcuuuucuuuuccugauuuccguagcagcggggagagggugaagccccugaaccccugaggcuccucggagacuucuugugguuccuguggugucaccccucagcccuaccugcgaaagaaaaaagaguuagaaggcggcagaggagcugacuggucgucgucgacgaa

SEQ ID NO: 687 - RNA, 327 nt, Artificial Sequence
  Synthetic Construct
uaaacaccgugcggucccugugguucgccccgccaaagcauauaauuaaagaaaacgaggagaagaaggaacucaccugucgucgucgacgaaggccggugagaaaggagcugccagcacuaagccggacggcgcccuygcaccaguuccucugugggcuugcccucaagaacgcuacgguguugaggcccuguccgccgcugcgcugcacrccucucuucgcgccgcuagucgagcggacuacgggcgguuucccuauucucucacgcucuuuucuuuuugugcagcggcggguggguucccaggguraaacacaauagguguuuc

SEQ ID NO: 688 - RNA, 323 nt, Artificial Sequence
  Synthetic Construct
cccauaaguuucgucgcaucccagcgacucaucagugggcuaagcccagacuuaugagagagugguuaccucucaggugcccuauucggagcacugcaguucccgauagaaaggcuaagcacuucgcaaugaaguaaggugggacuuuucccuaggguuugagcggucgaacccagggggagugugauccagguaccgccguagaaacuggauuacgacgccuacccgggauucaaacccggucccccucagaggugacucugagugaaaggucugugcuuagcacacugaygaguuccugaaauggaacgaaaccuuucuaa

SEQ ID NO: 689 - RNA, 308 nt, Artificial Sequence
  Synthetic Construct
cccauaaguuucgucgcaucccagcgacucrucagugggcuaagcccagacuuaugagagagugguuaccucucagccccuccaccuuggggugcccuauucgragcacugcaguuycgauagaaaggcuaagcacuucgcaaugaaguaaggugggacuuuucccuaggguuugagcggucgaaccccagguaccgccguagaaacuggauuacgacgccuacccggucccccucagaggugacucugagugaaaggucgugcuuagcacacugaygaguuccugaaauggaacgaaaccuuucuaa

SEQ ID NO: 690 - RNA, 308 nt, Artificial Sequence
  Synthetic Construct
ucgacggcgcgucggccuucucgcgcacucuguycgguacuacccgguggauacaacugacagaggugcuuuuucuuccacccrcuucuaccgacgcggccgggagugaagcuacccgggacccgagaggaucuccggauucuucuaagggugccuguggugccucccccgaagcccgcuuagggaaaaagaaaggggaagcaagcaucuccuguucagggauccccggggaaaccugracagaycgggcggagaagcgccggcgggccgucuucugacaggaruaauccccgcugaarggguuuuca

SEQ ID NO: 691 - RNA, 320 nt, Artificial Sequence; identical to SEQ ID NO: 275
  Synthetic Construct

SEQ ID NO: 692 - RNA, 317 nt, Artificial Sequence
  Synthetic Construct
ucggaaaucaggugugaaccacagguaccacgaguuuaguuccgaggaaccucugcgguuccaagggcuuaacucccuccccccgaagccaagagauagagaaaaagccggucucgggagcuucacuugguuccacccgguaauaacccaaacgaccgcgcaaagggggcgaggggugguccugcgggcgcgaggaaggacacccgaacaaaggaagggugaaaaccccgauucuuuuggaauuacuccuguaggccgcugggcacuccccaaaagaccuuauugccaguucgguucagguuuccccguggauaaaa

SEQ ID NO: 693 - RNA, 272 nt, Artificial Sequence
  Synthetic Construct
guggcgaaguugagcucugcucuucuaagcuguaaccggaccgguccccuucacccgagcgcugcuugccgcuagucgcgcggacuuccaagucucccucccgagccgcuuuucucuucaccuaauuuccguagcagcggggagagggugaagccccugaaccccugagggcuccucggagacuucuugugguuccuguggugucaccccucagcccuaccugcgaaagaaaaaaguguuagaaggcggcagaggagcugacuggucguccg

SEQ ID NO: 694 - RNA, 194 nt, Artificial Sequence
  Synthetic Construct
cuggggaauacacuacgugacuuaccuguauggggcaagggcucgaagagggauccccgggaaaccuacucgagcgaggcggagaucgagcgccaguucgugcgcggcgaccugaaguugcuucggcuucuucuuguucgcguccugcguggaacggcuccuucuucacaccagccggaguuggaaacccggur

SEQ ID NO: 695 - RNA, 313 nt, Artificial Sequence
  Synthetic Construct
uggugaacaaccuugguuccugugggucaccccccgaccacaaugcuuaaaagaaaaaucagrgagaggaagagacuuaccugucgucgucgacgaaggcugguaagccgaguuggcgaguagcgccgaguggaguaaagacggagaaccucucgaaagagacugacuccgcuuccuccggccgcucccgucuuccgagcuuccugccgcuagucgagcggacaacugguagcucucccacucucucagauuuucucuugagagcggggccgggagcggguccccaggggaaaacacgauugguguucuccac

SEQ ID NO: 696 - RNA, 310 nt, Artificial Sequence
  Synthetic Construct
cggagacuucuugugguuccuguggugcaccccucagcccuaccugcgaaagaaaaaagguuagaaggcggcagaggagcugacuggucgucgucgacgaaggcucgucagcugcggagguuggggucgacuggcuccgguggcgaaguugagcucugccuucuaagcuguaacggaccgguccccuucacccgagcgcugcuugccgcuagucggcggacuucaagucuyccucccgagccgcuuuucuuuucuccurauuuccguagcagcggggagagggugaagccccugaacccugagggcuccu

SEQ ID NO: 697 - RNA, 63 nt, Artificial Sequence
  Synthetic Construct
agcuuggcuucgcggcgaggguggaaguuuaccgcggacccccgagaggaggcccucgggucc

SEQ ID NO: 698 - RNA, 302 nt, Artificial Sequence
  Synthetic Construct
cuuuccugagguuccuguggugcuccccugaccugcguuccaaaaagcraaaaaagugagaggcccyaggggcuucucggcucgucgucgacgaagggucuagaagccugggygggagcgcgcggcugugaguaauygcuccucuggagaagaaaaccagcguugcuucycgccuccucgucuucugucccgcuagucgagcggacaacccgagcaccgccgaaggccuuuuuucuuuuragcagcuuggyuucgcggcgagggggaaguaccgcggacyccygrraggaggcccucggguc

SEQ ID NO: 699 - RNA, 316 nt, Artificial Sequence; identical to SEQ ID NO: 283
  Synthetic Construct

SEQ ID NO: 700 - RNA, 306 nt, Artificial Sequence
  Synthetic Construct
cuuuccugagguuccuguggugcuccccugaccugcguuccaaaaagcraaaaaagugagaggcccuaggggcuucucggcucgucgucgacgaagggucugaagccgggcgcuggcggagcgcgcggcugugaguaauygcuccuyuggagaagaaaaccagcguugcuuccygccugagccugucuucugucccgcuagucgagcggacaacccgagcaccgccgaagcuuuuuucuuuuaagcgcuuggyuucgcggcgaggguggaaguuuaccgcggacccccgagaggaggcccucrggu

SEQ ID NO: 701 - RNA, 301 nt, Artificial Sequence
  Synthetic Construct
cuuuccuugagguuccuguggurcuccccugaccugcauuccaaacgcaaaaaaagugagaggcccuaggggcuucucggcucgucgucracraagggucuaraagccgggcggcggagcgcgcggcugygrrurauygcuccuyuggaraagaaraccagcguugcuucyygccuccucguyuuyugucccgyuagucgagcggacaacccgagcacgccgargcyuuuuuyyuuuaagcugcuugguggugcggcgaggguggaagucaccgcggaucuccuagaaggggcccucgggu

SEQ ID NO: 702 - RNA, 127 nt, Artificial Sequence
  Synthetic Construct
yucrrauccragrggcyargagaggrccgcggygcucurgaarargcuugrraacrccaycgrucgyycuyyucacyrgugrrragrrcgaycgruggyruuucaacrragccucurcycrgacycc

SEQ ID NO: 703 - RNA, 315 nt, Artificial Sequence; identical to SEQ ID NO: 287
  Synthetic Construct

SEQ ID NO: 704 - RNA, 313 nt, Artificial Sequence
  Synthetic Construct
cuuuccuugagguuccugugguacuccccugaccugcauuccaaaacgcaaaaaaagugagaggcccuaggggcuucucggacucguugucgaauaagggccuagaagcugggucgggcgggagcgcgcggcugugaguaaucgcuccucuggagaagaaaaccagcguugcuucucgccugagccucguuuuuugucccguuagucgagcggacaacccgagccccgccgaaggccuuuuuuuuuuuaagcugcuugguggugcggcgaggguggaagucaccgcggaucuccuagaaggaggcccucgggu

SEQ ID NO: 705 - RNA, 314 nt, Artificial Sequence; identical to SEQ ID NO: 289
  Synthetic Construct

SEQ ID NO: 706 - RNA, 313 nt, Artificial Sequence; identical to SEQ ID NO: 290
  Synthetic Construct

SEQ ID NO: 707 - RNA, 312 nt, Artificial Sequence; identical to SEQ ID NO: 291
  Synthetic Construct

SEQ ID NO: 708 - RNA, 310 nt, Artificial Sequence
  Synthetic Construct
rgagaaaacuccgugugguuccuguggggcaccccccugygcuacugacuaaaagaaaaauucaacaggugaagagacuuaccugucgucgucgacgaaggcugguaagccgugaaggcguggaggcggcuccaggucgugccuuuuggaagaaacuccuaaaggacuugacuggaugcccgccugaccugcccgagcuuuccaccgcuagucgagcggacuaauggcaagcucuccgacccuuagaauuucuucuuaggucgggcagggaguggagccuccaggguaaaacacgauygguguuuucccc

SEQ ID NO: 709 - RNA, 296 nt, Artificial Sequence
  Synthetic Construct
cuggggaauucucgaguugccgcaurggcaagcaaagaaaaaacaaggcaggraggaracuuaccugagaaaggagccccggggcaacucuucucagaauccagcggrggcguggagagagggccgcggugcucuggaguagaggcuucugcuucgaaacaccaucgaucgucccuucuucuuuaccuycuycuggcucuucrrugagacgcgaccgguggcaucaccucucgguucgucuccaaccugcuuuuugucuaucugagccucugccgcggauccucucuugagccccu

SEQ ID NO: 710 - RNA, 40 nt, Artificial Sequence
  Synthetic Construct
rraggccycurcyygrcygucyrcgccyrgaucygrrarr

SEQ ID NO: 711 - RNA, 306 nt, Artificial Sequence
  Synthetic Construct
ggaggaaaacuccgugugguuccuguggggcaccccccugcgcaaaugacuaaaagaaaaaucagcaggugaagagacuuaccugucgucgucgacgaaggcugguaagccgugacgcguggaggagagcccgggucgcgcucuuggggguaaccccuuugagacuugaccgguuccucucccacccugccgagcuuccugccgcuagucgagcggacuccggguagcucuccgacccucagauuuucucuuaaggucuggcagggaguggagcucccaggguaaaacacrauygguguuuucccc

SEQ ID NO: 712 - RNA, 306 nt, Artificial Sequence; identical to SEQ ID NO: 296
  Synthetic Construct

SEQ ID NO: 713 - RNA, 306 nt, Artificial Sequence; identical to SEQ ID NO: 297
  Synthetic Construct

SEQ ID NO: 714 - RNA, 137 nt, Artificial Sequence
  Synthetic Construct
agraucrgcgagaryyagagrggycggggucygrgyagaggcucgyraaaacagrugrgacgcgaggugryrycaccucgcgucycacuguuurcgagccucurcycrgacyccgrccycucurryucucgcggryc

SEQ ID NO: 715 - RNA, 289 nt, Artificial Sequence
  Synthetic Construct
cuggggaauucucgguugccgcacaaggcaagcaaaraaaaaacaaggcagggaggagacuuaccugagaaaggagccccggggcaacucuucucagaauccagcrgaggcguggagaagggcccggugcucuggaguagaggcucugucuucgaaacaccaucgaucgucccuucucuuuaccuuccuucggcucuucgagugagacggaccgguggcacccuccugggucuccacccugcuuuuuguucuaucugagccucugccgcggauccucucuugagccccu

SEQ ID NO: 716 - RNA, 304 nt, Artificial Sequence; identical to SEQ ID NO: 300
  Synthetic Construct

SEQ ID NO: 717 - RNA, 296 nt, Artificial Sequence
  Synthetic Construct
ggaaaacuuccgugugguuccuguggggcacccccccgcgcagugcuuaaaagaaaauucaguaggugaagagacuuaccugucgucgucgacgaaggcugguaagccgacgcguggauggggacccgggucgcgcucuuggaggaaacuccuuugagacuugaccggcgagccuucuccugcagagcuccugccgcuagucgagcggacaacggcagcucuccgacccucagaauuucucuucaggccgggcggggauggagcucccaggguaaaacacaaucgguguuuucccc

SEQ ID NO: 718 - RNA, 296 nt, Artificial Sequence
  Synthetic Construct
cuggggaauucucgaguugccgcaaaaggcaugcaaagaaaaaaacuaggcagggaggygcuuaccugagaaaggagccccggggcaacucuucucagaauccagcragaggcguggagagagggccgcggugcucuggaguagaggcucugcuucgaaacaccaucgaucgucccuucuucuuuaccuucuucuggcucuucuuggagacgcgaccgguggcaccccugcucgguucgcuccaaccugcuuuuguucuaucugcgccucugccgcggauccucucuugagccccu

SEQ ID NO: 719 - RNA, 287 nt, Artificial Sequence
  Synthetic Construct
cuggggaauucucgaguugccgcaaggcagcaaagaaaaaacggcaggraggurcuuaccugagaaaggagccccggggcaacucuucucagaauccagcgaggcguggagagagggccgcggugcucuggaguagaggcucugccuucgaaacaccaucgaucgucccuucuucuuuaccuucuycuggcucuuccgaugagacgcgaccgguggcaucaccucucgguucgucccaaccugcuuuuucuaucugagccucugccgcggaccucucuugagccccu

SEQ ID NO: 720 - RNA, 277 nt, Artificial Sequence
  Synthetic Construct
cuggggaauucucgaguugccgcaurgcaagcaaagaaaaaacaaggcacuuaccugagaaaggagccccggggcaacucuucucagaauccagcgagaggcguaggagagagggccgcggugcucuggaguagaggyucuucgaaacaccaucgaucgucccuucuucuuuuaccuucuucuggcucuucugagacgcgaccgguggcaucaccucucgguucgucccugcuuuuuucuaucugagccucugccgcggauccucucuugagccccu

SEQ ID NO: 721 - RNA, 291 nt, Artificial Sequence
  Synthetic Construct
cuggggaauucucgaguugccgcauggccaagcaaagaaaaaacaggcagggaagacuugcuugaraaagcagccccggggcacucuucucugaaucccgagaggcguaggcgagagggccgguugcucuggaguagaggcuucuugcuucgaaacaucaucgucgucccuucuucuuuuaccuucuccuggcucuuccgaugagacgcgaccgguggcaucaccucucgguucgucuuccaaccugcuuuuugucuaucyaagccuugccgcggucucucuugagccccu

SEQ ID NO: 722 - RNA, 289 nt, Artificial Sequence
  Synthetic Construct
cuggggaauucucgaguugccgcaurggcaagcaaagaaaaaaarggcaggrrrgrracuuaccugagaaaggagccccggggaacucuucucagaauccagcggrggcguggargrgggccgcggugcucuggaguagaggcuucugcuucgaaacaccaucgaucgucccuucuucuuuaccuycycugryucuucygugagacgcgaccgguggcaucaccucucgguucgyycaaccugcuuuugucuaucugagccucugccgcggauccucucuugagccccu

SEQ ID NO: 723 - RNA, 302 nt, Artificial Sequence; identical to SEQ ID NO: 307
  Synthetic Construct

SEQ ID NO: 724 - RNA, 299 nt, Artificial Sequence
  Synthetic Construct
cggaucauuuucuugugguuccugugguacaccuccgaaggcgcgcuaggcgagaagagaguaaauagaggagagagcuuaccuggguucccuggcagygcugcaacggaauycagugcccaccggagcgccaguucgugcgagagccgcuucggcuucuucuycgcggaacggcucgcguguacgccugggaccccgcragcgauugcaaaggucccgguucgcucuccuuuucuuuuuuaccuacuycagcgaccgccccggggagugaaucacccgggaccgcgaaaagcgauccu

SEQ ID NO: 725 - RNA, 300 nt, Artificial Sequence; identical to SEQ ID NO: 309
  Synthetic Construct

SEQ ID NO: 726 - RNA, 298 nt, Artificial Sequence
  Synthetic Construct
cuggggaauucucgaguugccgcaucaggcaagcaaagaaaaaacaaggcagggagguacuuaccugagaaaggagccccggggcacgacucuucucagaauccauaccuagagaggugcgcacggcggaggugcucuggauacaagcucugccuucgaaacaccaucgaucgugccuucuucuuuaccuucuucuggcucuuccgaugagacgcgaccgguggcaucaccucucgguucgucccaaccugcuuuuugucuaucugagccucugccgcggauccucucuugagccccu

SEQ ID NO: 727 - RNA, 294 nt, Artificial Sequence
  Synthetic Construct
cuggggaauucucgaguugccgcauaggcaagcaaagaaaaaacaagggcaggagagacuuaccugagaaaggagccccggggcaacucuucucagaauccagcgggggcguggagagagggccgcggugcucuggaguagaggcucugcuucgaaacaccaucgaucgucccuucuucuuuaccuccuucuggcucuuccgaugagacgcgaccgguggcaucaccucucgguucguccraccugccuuuuugucuaucugagccucugccgcggauccucucuugagccccu

SEQ ID NO: 728 - RNA, 292 nt, Artificial Sequence
  Synthetic Construct
gaggaaacuccgugugguuccuguggggcacacccccuugccgaaaauaaaaygcagagagggaaagggraacuuaccugucgucgucgacgaaggcagcuaaguuggugacgccgaguggaguaaagacggagagycuccgcuagucggaaagacuccgcauccuccggcagacccuycuagcuccgcuagucgagcggacaacugagugaguygycccaauccuaaucuguuuuuauyuaggcuagraggggauugggccuccaggguaaaacacgauugguguuucccc

SEQ ID NO: 729 - RNA, 293 nt, Artificial Sequence
  Synthetic Construct
cuggggaauucucgaguugccgcaugaggcaagcaaagaaaaaacuaggcagggaggcgcuuaccugagaaaggagccccggggcaaccucuucucagaauccagcggaggcguggagagagggccgcggugcucuggaguagaggcuucugcuucgaaacaccaucgaucgucccucuucuuuaccuuccuggcucuucgagugagacgcgaccgguggcaccccugcugguucgcuccaccugcuuuuuguucuaucugcgccucugccgcggauccucucuugagccccu

SEQ ID NO: 730 - RNA, 294 nt, Artificial Sequence
  Synthetic Construct
ggaggaaacuccgugugguuccuguggggcacacccccuugccgaaaauaaaugcagagaggggaaaggraacuuaccugucgucgucgacgaaggcagcuaaguuggugagccgaguggaguaaagacggagarycuccgcuagucggaaagacuccgcuccuccggcugacccuccuucagcucgccgcuagucgagcggacaacugagugaguagccccaacccuaaucuguuuuuaucuaggcuagaaggggauugggccuccaggguaaaacacgauugguguuucccc

SEQ ID NO: 731 - RNA, 297 nt, Artificial Sequence; identical to SEQ ID NO: 315
  Synthetic Construct

SEQ ID NO: 732 - RNA, 64 nt, Artificial Sequence
  Synthetic Construct
cuggggaaaucuacagggcccccaaaaacyacugcaggagaggccgcuugagggauccccgggg

SEQ ID NO: 733 - RNA, 297 nt, Artificial Sequence; identical to SEQ ID NO: 317
  Synthetic Construct

SEQ ID NO: 734 - RNA, 297 nt, Artificial Sequence; identical to SEQ ID NO: 318
  Synthetic Construct

SEQ ID NO: 735 - RNA, 297 nt, Artificial Sequence; identical to SEQ ID NO: 319
  Synthetic Construct

SEQ ID NO: 736 - RNA, 297 nt, Artificial Sequence; identical to SEQ ID NO: 320
  Synthetic Construct

SEQ ID NO: 737 - RNA, 296 nt, Artificial Sequence; identical to SEQ ID NO: 321
  Synthetic Construct

SEQ ID NO: 738 - RNA, 279 nt, Artificial Sequence
  Synthetic Construct
ggaaaacuccgugugguuccuguggggcacacccccuugccgaaaauaaaacgcagagagggaaaaggraacuuaccugucgucgucgacgaaggcagcuaaguuggugacgccgaguggaguaaagacggagagucuccgcuagucggaaagacuccgcauccuccggcgaccccugcgccgcuagucggcggaccgagugcccyaauccuaaucuguuuuuaucuaggcuagaaggggauugggccuccaggguaaaacacgauugguguuuucccc

SEQ ID NO: 739 - RNA, 296 nt, Artificial Sequence; identical to SEQ ID NO: 323
  Synthetic Construct

SEQ ID NO: 740 - RNA, 295 nt, Artificial Sequence; identical to SEQ ID NO: 324
  Synthetic Construct

SEQ ID NO: 741 - RNA, 287 nt, Artificial Sequence
  Synthetic Construct
ugggugaacaaccuugugguuccugugggucaccccgcccyggaauaauaaaagcagaggagagaraguacucaccugucgucgucgacgaaggccggugagcaguaagccggacgguccccucgcggccguccucuggagcucugcucuaagacuucgyygcugaggcccgcgccgccgcucuuccgcgcugcuagucgagcggacguuggurgucucccucucccuugcaauaaaauccrgyggcgagugguguccccaggguaaaacacgauugguguucuccc

SEQ ID NO: 742 - RNA, 285 nt, Artificial Sequence
  Synthetic Construct
ugggugaacaaccuugugguuccugugggucaccccgccccacggaaauaaaagcagaggagagaraguacucaccugucgucgucgacgaaggccggugagcaguaagccggacggcccucgcggccgauccucuggagcuyygcucuagaucuucgcugcugaggcccgcgccgccgcucucgcgcygcuagucgagcggacguugguggucuyycucucccugugcaauaaaayccaggggcgagygguguccccaggguaaaacacgauugguguucuccc

SEQ ID NO: 743 - RNA, 281 nt, Artificial Sequence
  Synthetic Construct
ggaaaacuccgugugguuccuguggggcacacccccuugccraaaauaaaaygcagagagggraarggraacuuaccugucgucgucgacgaaggcagcuaaguuggugacgcacuguggaaucgcccuugucuccgcuagucggaaagacuccgcauccuccggcgacccyyagcucccgcuagucgagcggacyuagugagugyccyaayccuaaucuguuuuuaucuaggcuagaaggggauugggccuccaggguaaaacacgauugguguuuyccc

SEQ ID NO: 744 - RNA, 294 nt, Artificial Sequence; identical to SEQ ID NO: 328
  Synthetic Construct

SEQ ID NO: 745 - RNA, 294 nt, Artificial Sequence; identical to SEQ ID NO: 329
  Synthetic Construct

SEQ ID NO: 746 - RNA, 293 nt, Artificial Sequence; identical to SEQ ID NO: 330
  Synthetic Construct

SEQ ID NO: 747 - RNA, 293 nt, Artificial Sequence; identical to SEQ ID NO: 331
  Synthetic Construct

SEQ ID NO: 748 - RNA, 293 nt, Artificial Sequence; identical to SEQ ID NO: 332
  Synthetic Construct

SEQ ID NO: 749 - RNA, 287 nt, Artificial Sequence
  Synthetic Construct
aaaaagugccccucuggggaauacacaacguuacuuacuugaauguugcaaagggcucgaagagggauccccggggaaaccuacucgagcgaggcggagaucgagcgccaguucgugcgcggcgaccugaaguugcuucggcuucuucuaguucgcgaccugcguggaacggcuccuucuuccaccagccggagugaaacuacgguggauacaacucuugagcgccgagcuuaaccugcagaaguucacauaaaaagugcauauaguaagagcuuaguguagggaaa

SEQ ID NO: 750 - RNA, 128 nt, Artificial Sequence
  Synthetic Construct
gyrgrrgcucgaagaggaucccgrgayccacucrgcgrgygraggagccruycrygcrrgacgaagyrcuucrgcuyccuygyucggurgucccggccyucuucrcaccacrggaaryyacyrugauc

SEQ ID NO: 751 - RNA, 146 nt, Artificial Sequence
  Synthetic Construct
aaccacacggaguyuccuccggggaaacaccaaucguguuuuacccuggaggcccaauccccuucuagccuagauaaaaacagruuaggruugggrcacucacucaguuguccgcucgacuagcgggagcuagragggucgccgga

SEQ ID NO: 752 - RNA, 289 nt, Artificial Sequence; identical to SEQ ID NO: 336
  Synthetic Construct

SEQ ID NO: 753 - RNA, 287 nt, Artificial Sequence; identical to SEQ ID NO: 337
  Synthetic Construct

SEQ ID NO: 754 - RNA, 269 nt, Artificial Sequence
  Synthetic Construct
cuggggaaucuccrgaccaaaaaaacagcuugggagggaacauaccugaagagggauccccggggaaaucucuucagacucgucgaggggagggcgccgcggaucacuggcguccagcaccggaacaggagcucgucuccuuccuuccaycgcuggcuccacauccgaucgucgcuucuuccuucgcgaccugagauagaaacuacccgguggauacaacucuuggguuguuccucccaggcuuguuaaaauggcccguugagaccccu

SEQ ID NO: 755 - RNA, 285 nt, Artificial Sequence; identical to SEQ ID NO: 339
  Synthetic Construct

SEQ ID NO: 756 - RNA, 285 nt, Artificial Sequence; identical to SEQ ID NO: 340
  Synthetic Construct

SEQ ID NO: 757 - RNA, 284 nt, Artificial Sequence; identical to SEQ ID NO: 341
  Synthetic Construct

SEQ ID NO: 758 - RNA, 284 nt, Artificial Sequence; identical to SEQ ID NO: 342
  Synthetic Construct

SEQ ID NO: 759 - RNA, 283 nt, Artificial Sequence; identical to SEQ ID NO: 343
  Synthetic Construct

SEQ ID NO: 760 - RNA, 281 nt, Artificial Sequence; identical to SEQ ID NO: 344
  Synthetic Construct

SEQ ID NO: 761 - RNA, 280 nt, Artificial Sequence
  Synthetic Construct
uggcaauuguucucagaauccagcgagaggcguaggagagagggccgcggugcucuggaguagaggcuucuugcuucgaaacaccauccaucguccguucuucuuuuaccuucuccuggcucuucgarugagacgcaccgguggcaucaccucucgguucgucuuccaaccugcuuuuuggcuaucugagccucugccgcggauccucucuugagccccucuggggaauucucgacuugccgcaugggcaagcaaaaaaaaagcaaggcaagaaugacac

SEQ ID NO: 762 - RNA, 272 nt, Artificial Sequence
  Synthetic Construct
agaguuguuuccaccggguaguuuccaacuccgucuugugugaagaaggagccguuccacgcaggacgcgaacaagaagaagccgaagcaacuucagggcgccgcgcacgaacuggcgccgacuccgccugcucgguagguuccccggggaucccucuucgagcccuugccaccaucccgguaagucacguaggguauuccccagaggggcacuuuuuaugugaacuucugcagguuaagcucggcgcucaagaguuguauccaccggguag

SEQ ID NO: 763 - RNA, 264 nt, Artificial Sequence
  Synthetic Construct
cuggggaauuucucucgggccaaaaaaacagcuugaggagggaacuaaccugaagagggauccccggggaaaucucuucagacguugaggggagggcgccgcggaucacuggcgagcagaaacaggagcucgucuccuuccuuucuugccuccacauccgaucgucgcuucucyyuuccaccugaaaaagaaacuacccgguggauacaacucuuuaagguuguuccucccaggcuuguuaaaaauggcccgcuugagaccccu

SEQ ID NO: 764 - RNA, 277 nt, Artificial Sequence; identical to SEQ ID NO: 348
  Synthetic Construct

SEQ ID NO: 765 - RNA, 276 nt, Artificial Sequence; identical to SEQ ID NO: 349
  Synthetic Construct

SEQ ID NO: 766 - RNA, 276 nt, Artificial Sequence; identical to SEQ ID NO: 350
  Synthetic Construct

SEQ ID NO: 767 - RNA, 275 nt, Artificial Sequence; identical to SEQ ID NO: 351
  Synthetic Construct

SEQ ID NO: 768 - RNA, 274 nt, Artificial Sequence
  Synthetic Construct
ugaccucaauugacuagaacaguaguaaagaaaaggcagguugcuuaccuggcuucccuggcagcgcugcaacggaauucagggcacggagucggugccagaucgugcggcuucggcgccaguucgugcgagagcuucgcucgcgaacucuacguggaacggccgucgcucguggrucggccgucuuccggguuccugcucccacgcggcgauugcaaaggagcugguucgcguccuguccuauucuucuacucuaguucaacuuaaagagguu

SEQ ID NO: 769 - RNA, 273 nt, Artificial Sequence; identical to SEQ ID NO: 353
  Synthetic Construct

SEQ ID NO: 770 - RNA, 272 nt, Artificial Sequence; identical to SEQ ID NO: 354
  Synthetic Construct

SEQ ID NO: 771 - RNA, 270 nt, Artificial Sequence; identical to SEQ ID NO: 355
  Synthetic Construct

SEQ ID NO: 772 - RNA, 270 nt, Artificial Sequence; identical to SEQ ID NO: 356
  Synthetic Construct

SEQ ID NO: 773 - RNA, 270 nt, Artificial Sequence; identical to SEQ ID NO: 357
  Synthetic Construct

SEQ ID NO: 774 - RNA, 113 nt, Artificial Sequence
  Synthetic Construct
cgaagaggaucccggaccaccrgcggygragaggccruycrygcrgrgcgaagrcrgccuucgucgycygcrygryggcucucuycrccycygaguggyuccgggauccucuu

SEQ ID NO: 775 - RNA, 134 nt, Artificial Sequence
  Synthetic Construct
cgagcggacguugguggucucccucucccugugcaauaaaayccagguggcgagugguguccccaggguaaaacacgauugguguucucccugggugaacaaccuugugguuccugugggucaccccgccccac

SEQ ID NO: 776 - RNA, 268 nt, Artificial Sequence; identical to SEQ ID NO: 360
  Synthetic Construct

SEQ ID NO: 777 - RNA, 258 nt, Artificial Sequence
  Synthetic Construct
caacucuuaagcgccgagcuuaaccugcagaaguucacauaaaaagugccccucuggggaauacacgacgggacuuaccuguaugguggcaaggccgaagagggaccccggggaaccuacucgagcgaggcggagagcgagcgccaguucgugcgcggcgccugaguugcucggcucucuuguucgcgucugcguggaacggcuccuucuucacaccagccggaguuggaaacaaccggguggauacagcucuagagc

SEQ ID NO: 778 - RNA, 266 nt, Artificial Sequence; identical to SEQ ID NO: 362
  Synthetic Construct

SEQ ID NO: 779 - RNA, 265 nt, Artificial Sequence; identical to SEQ ID NO: 363
  Synthetic Construct

SEQ ID NO: 780 - RNA, 264 nt, Artificial Sequence; identical to SEQ ID NO: 364
  Synthetic Construct

SEQ ID NO: 781 - RNA, 264 nt, Artificial Sequence; identical to SEQ ID NO: 365
  Synthetic Construct

SEQ ID NO: 782 - RNA, 264 nt, Artificial Sequence; identical to SEQ ID NO: 366
  Synthetic Construct

SEQ ID NO: 783 - RNA, 158 nt, Artificial Sequence
  Synthetic Construct
cacucygcygrgyaagagragyyryyaragrcccyrrgaracagyracuucygyryrrrcrggcucgaagrgguccccggggaccuyucgagccygccauyryrgyaaguyrcuguyuycyygcrcuyyurygugracuucucccccrgaguugaccc

SEQ ID NO: 784 - RNA, 263 nt, Artificial Sequence; identical to SEQ ID NO: 368
  Synthetic Construct

SEQ ID NO: 785 - RNA, 215 nt, Artificial Sequence
  Synthetic Construct
cgaccacgcaggaccgaacaaaagaagccgagcaacuucagucgccgcgcacgaacugcgcucgaucuccgccucgcucgaguagcuuuccccggggaucccucuucgcgcccuugccaccauacagguaagucacguaguguauuccccagaggggcacuuuuuaugugaacuucugcagguuaagcucggcgcucaagaguuguauccaccgg

SEQ ID NO: 786 - RNA, 259 nt, Artificial Sequence; identical to SEQ ID NO: 370
  Synthetic Construct

SEQ ID NO: 787 - RNA, 258 nt, Artificial Sequence; identical to SEQ ID NO: 371
  Synthetic Construct

SEQ ID NO: 788 - RNA, 258 nt, Artificial Sequence; identical to SEQ ID NO: 372
  Synthetic Construct

SEQ ID NO: 789 - RNA, 258 nt, Artificial Sequence; identical to SEQ ID NO: 373
  Synthetic Construct

SEQ ID NO: 790 - RNA, 257 nt, Artificial Sequence; identical to SEQ ID NO: 374
  Synthetic Construct

SEQ ID NO: 791 - RNA, 257 nt, Artificial Sequence; identical to SEQ ID NO: 375
  Synthetic Construct

SEQ ID NO: 792 - RNA, 223 nt, Artificial Sequence
  Synthetic Construct
cgcagaccaacaagaagagccgaagcaacuucaggcgccgcgcacgaacuggcgcucgaucuccgccucgcucgaguagguuuccccggggaucccucuucgagcccuugcaccauaagguaagucacguaguguauuccccagaggggcacuuuuuaugugacuucugcagguuaagcucggcgcucaagaguuguauccaccggguaguuucaacuccggu

SEQ ID NO: 793 - RNA, 256 nt, Artificial Sequence; identical to SEQ ID NO: 377
  Synthetic Construct

SEQ ID NO: 794 - RNA, 225 nt, Artificial Sequence
  Synthetic Construct
cuccggcuggagugaagaaggagacguucaacgcaggacgcgaacaagaagaagccgaagcaacuucaggucgccgcgcacgaacuggcgcucgaucuccgccucgcucgaguagguuucccgggggaugccucuucgagcccuugccaccaacaggaagucacguagguauucggagaggggcacuuuuuaugugaacuucugcagguuaagcucggcgcucaa

SEQ ID NO: 795 - RNA, 256 nt, Artificial Sequence; identical to SEQ ID NO: 379
  Synthetic Construct

SEQ ID NO: 796 - RNA, 236 nt, Artificial Sequence
  Synthetic Construct
crcuaygugacuuacyrrugruggcaagggyucgaagagggauccccggggaaaccuacucgagcgaggcggagaucgagcgccaguucgugcgcggcgaccugaagugcuucggcuucuucuuguugcguccugcguggaayggcuccuucuycacaccagccggaguuggaaacuacccgguggauacaacucuugagcgccgagcuuaccugcaraaguucayruaarrygyu

SEQ ID NO: 797 - RNA, 255 nt, Artificial Sequence; identical to SEQ ID NO: 381
  Synthetic Construct

SEQ ID NO: 798 - RNA, 255 nt, Artificial Sequence; identical to SEQ ID NO: 382
  Synthetic Construct

SEQ ID NO: 799 - RNA, 120 nt, Artificial Sequence
  Synthetic Construct
ugargagcyuucygcagrycayaaraagrccgaaacucrygcyrcygayggcgrgcucgargguccggggaccuyucgagyccrucgyaguyrcuguyuycyygcrcuyyurygugracu

SEQ ID NO: 800 - RNA, 254 nt, Artificial Sequence; identical to SEQ ID NO: 384
  Synthetic Construct

SEQ ID NO: 801 - RNA, 236 nt, Artificial Sequence
  Synthetic Construct
uuccaacuccggcuggugugaagaggagccguuccacgcaggcgcgaacaagagaagccgaagcaacuucggcgccgcgcacgaacuggcgcucgagcuccgccucgcucgagucgguuuccccggggaucccucuucggcccuugccccauacagguaagucacguaguguauuccccagaggggcacuuuuuaugugaacuucugcagguuaagcucggcgcucaagaucggag

SEQ ID NO: 802 - RNA, 254 nt, Artificial Sequence; identical to SEQ ID NO: 386
  Synthetic Construct

SEQ ID NO: 803 - RNA, 253 nt, Artificial Sequence; identical to SEQ ID NO: 387
  Synthetic Construct

SEQ ID NO: 804 - RNA, 252 nt, Artificial Sequence; identical to SEQ ID NO: 388
  Synthetic Construct

SEQ ID NO: 805 - RNA, 160 nt, Artificial Sequence
  Synthetic Construct
yuccruugygrgcucgaagaggaucccgggarccacucrgcgrgygragagagccrucygcrgucgacgaagugcyrgcuucguycgcgcraugggcucucuycrccygcygaguggyucycgggauccucuurrrccgcyuaayyyrcgrrucrgargr

SEQ ID NO: 806 - RNA, 252 nt, Artificial Sequence; identical to SEQ ID NO: 390
  Synthetic Construct

SEQ ID NO: 807 - RNA, 170 nt, Artificial Sequence
  Synthetic Construct
uuaaraacaaugargauagauaaacyuugygacacrguyyuyccrcuuucccugagcgaugragarrgragcgrgugaacuuuuaaaaaguucacycgcuycyyucuycaucgcucagggaaagyggrarracygugucrcaarguuuaucuaucyucayuuuuuyuaaa

SEQ ID NO: 808 - RNA, 251 nt, Artificial Sequence; identical to SEQ ID NO: 392
  Synthetic Construct

SEQ ID NO: 809 - RNA, 251 nt, Artificial Sequence; identical to SEQ ID NO: 393
  Synthetic Construct

SEQ ID NO: 810 - RNA, 251 nt, Artificial Sequence; identical to SEQ ID NO: 394
  Synthetic Construct

SEQ ID NO: 811 - RNA, 251 nt, Artificial Sequence; identical to SEQ ID NO: 395
  Synthetic Construct

SEQ ID NO: 812 - RNA, 237 nt, Artificial Sequence
  Synthetic Construct
cuggggaaaucuacagggacccyaaaaacyacucaggagaggccgcuugagggauccccggggaaacucaaucugggaagggagcguaccugggucgaucgugcgcguuggaggagacuccuucguagcuugacgcccggccgccccuccucgaccgcuugggagacuacccgguggauacaacucacgcggcucuuaccuguuguuaguaaaaaaaggugucccuuuguagccccu

SEQ ID NO: 813 - RNA, 251 nt, Artificial Sequence; identical to SEQ ID NO: 397
  Synthetic Construct

SEQ ID NO: 814 - RNA, 250 nt, Artificial Sequence; identical to SEQ ID NO: 398
  Synthetic Construct

SEQ ID NO: 815 - RNA, 244 nt, Artificial Sequence
  Synthetic Construct
uuucgucgacgucgaccaucagcuccucugccgcuuuauaacucuyyuyucuuucgcagguagggcugaggggugacaccacaggaaccacaagaagucucgrggagcccucgagggguucgaggggcuycacccucuccccgcugcuacggaauucgugacaacyaaagccgcuagcyaguaguruacgucacgauugaagaguccgcgcgacuaacgaccagcauggcuggggugaacgaaa

SEQ ID NO: 816 - RNA, 250 nt, Artificial Sequence; identical to SEQ ID NO: 400
  Synthetic Construct

SEQ ID NO: 817 - RNA, 246 nt, Artificial Sequence
  Synthetic Construct
uggcucgaacugacuagaacgguuaagaaaaaguucuyaccuggaguugcuuaccuggguucccuggcagcgcugcaacggaauccagugcccaccggagcgccaguucgugcgagaguugcuucggcuucuucuucguggaacgcuccgcguguacgccuggugacccgcgagcgauugcaaagguccugguucgcugacccgguuygaaccucuuuucuaaacccuaguucagcuuaacgagcu

SEQ ID NO: 818 - RNA, 244 nt, Artificial Sequence
  Synthetic Construct
ugugagagaaggaggagucguggugaacuuuuauuaaaaaaauaruucacucgucuucaaucucuugaucacuucgucucuucggaaagaugggaagaacacugaugagucucgcaarguuuauuccucuaucuucauuguuuuuuuacaaaaucyuguuauuaaacaagaagugaggauaugauuaaacuuuguuugacgaaaccaggucuguuccgacuuuccgacucugaguuucgacuua

SEQ ID NO: 819 - RNA, 247 nt, Artificial Sequence; identical to SEQ ID NO: 403
  Synthetic Construct

SEQ ID NO: 820 - RNA, 247 nt, Artificial Sequence; identical to SEQ ID NO: 404
  Synthetic Construct

SEQ ID NO: 821 - RNA, 246 nt, Artificial Sequence; identical to SEQ ID NO: 405
  Synthetic Construct

SEQ ID NO: 822 - RNA, 246 nt, Artificial Sequence; identical to SEQ ID NO: 406
  Synthetic Construct

SEQ ID NO: 823 - RNA, 246 nt, Artificial Sequence; identical to SEQ ID NO: 407
  Synthetic Construct

SEQ ID NO: 824 - RNA, 246 nt, Artificial Sequence; identical to SEQ ID NO: 408
  Synthetic Construct

SEQ ID NO: 825 - RNA, 244 nt, Artificial Sequence; identical to SEQ ID NO: 409
  Synthetic Construct

SEQ ID NO: 826 - RNA, 243 nt, Artificial Sequence; identical to SEQ ID NO: 410
  Synthetic Construct

SEQ ID NO: 827 - RNA, 243 nt, Artificial Sequence; identical to SEQ ID NO: 411
  Synthetic Construct

SEQ ID NO: 828 - RNA, 185 nt, Artificial Sequence
  Synthetic Construct
aaugcugggcuagaaacaggccggccagcgcucaagcccacaccugacaugcugccugauccuaccaacccauauaacaaacccucauuagaugcuuuguugaugauaaagccaagaguugugucuaacaauauggcuaccucugaggauaugaugaagaucugcguugaucuugagggguuggg

SEQ ID NO: 829 - RNA, 199 nt, Artificial Sequence
  Synthetic Construct
uggugugaagaaggagccguuccacgcaggucgcgaacaagaugaaggcguagcaaguucaggucgccgcgcacgaacuggcgcucgaucuccgccucgcucgaguagguuuccccggggaucccuguucggcccuugccaccuacagguaagucaguaguguauuccccaaggggacuuuuuaugugaacuucugcag

SEQ ID NO: 830 - RNA, 241 nt, Artificial Sequence; identical to SEQ ID NO: 414
  Synthetic Construct

SEQ ID NO: 831 - RNA, 217 nt, Artificial Sequence
  Synthetic Construct
uacgugacuuaccuguaugggggcaagggcucgaagagggauccccggggaaaccuacucgaacgaggcggagaucgagcgccaguucgugcgcggcgaccugaaguugcuucggcuucuucuuguuagcguccuucguggaacggcuccuucucacaccagccggaguuggaauacccgguggauacacucuugaccgagcuuaaccurcagaauu

SEQ ID NO: 832 - RNA, 234 nt, Artificial Sequence
  Synthetic Construct
gcgccguucgugcgcggcgaccugaaguugcuucggcuucuucuuguucgcguccugcguggaacggcuccuucuucacaccagccggaguuggaaacuacccgguggauacaacucuugagcgccgagcuuaaccugcagaaguucacauaaaaagugccccucuggggaauacacuacgugacuuaccuguaugguggcaagggcucgaagagggauccccggggaaaccag

SEQ ID NO: 833 - RNA, 229 nt, Artificial Sequence
  Synthetic Construct
gcgccagaguuguuccaccgguguuuccaacuccgcuggugugaagaaggagccguuccacgcaggacgcgaacaagaagacgccgaagcaacuucaggucgccgcccgaacuggcgcgcgaucuccgcucgcucgaguagguuuccccggggaucccucuucgagcccuugccaccauacagguaaguaacguaguguauuccccagcggggcacuuuuuaugugaac

SEQ ID NO: 834 - RNA, 235 nt, Artificial Sequence
  Synthetic Construct
acgcgaacaagaagaagccguagcuacuucagggcgcggcgcacgaacuggcgcucgauauccgccucgcucgaguagguuuccccggggaucccucuuagagcuugccaccauacagguaaguaacguaguguauuccccagaggggcacuuuuuauuugaacuucugcagguuaagcucggcgcucaagaguuguauccaccggguaguuuccaacuacggcuggugugaaga

SEQ ID NO: 835 - RNA, 200 nt, Artificial Sequence
  Synthetic Construct
cgucaagcgaaucugggaagggagguaccacccugcaucgugcgcguuggaggagacuccuucguagcuuagacgcccggccgccccuccucgaccgcuugggagacuacccgguggauacaacucacgcggcucuuaccuguuguuaguaaaaaaaggugucccuuuguagccccucugggaaaucuacaggcaccccc

SEQ ID NO: 836 - RNA, 139 nt, Artificial Sequence
  Synthetic Construct
cugcgyuyruuucgcagycrrgracycaggaguccycaaycyaaucuuuuayccurgargaucyucyaggruaaaagauurgruugrggacuccugrguycyygrcugcgaaayrarcgcagrgrgrargraryyuccr

SEQ ID NO: 837 - RNA, 233 nt, Artificial Sequence; identical to SEQ ID NO: 421
  Synthetic Construct

SEQ ID NO: 838 - RNA, 233 nt, Artificial Sequence; identical to SEQ ID NO: 422
  Synthetic Construct

SEQ ID NO: 839 - RNA, 233 nt, Artificial Sequence; identical to SEQ ID NO: 423
  Synthetic Construct

SEQ ID NO: 840 - RNA, 232 nt, Artificial Sequence; identical to SEQ ID NO: 424
  Synthetic Construct

SEQ ID NO: 841 - RNA, 232 nt, Artificial Sequence; identical to SEQ ID NO: 425
  Synthetic Construct

SEQ ID NO: 842 - RNA, 231 nt, Artificial Sequence; identical to SEQ ID NO: 426
  Synthetic Construct

SEQ ID NO: 843 - RNA, 229 nt, Artificial Sequence
  Synthetic Construct
cggagacuucuugugguuccuguggugacaccccucagcccuaccgcgaaagaaaaaaagaguuagaaggcggcagaggagcugacuggucgucgucgauccccuucacccgagcgcugcuugccgcuagucgagcggacuuccaagucucccucccgcgccgcuuuucuuuucuccgauucuguagcagcggggagagggugaagccccugaaccccugagggcuccu

SEQ ID NO: 844 - RNA, 230 nt, Artificial Sequence; identical to SEQ ID NO: 428
  Synthetic Construct

SEQ ID NO: 845 - RNA, 228 nt, Artificial Sequence; identical to SEQ ID NO: 429
  Synthetic Construct

SEQ ID NO: 846 - RNA, 228 nt, Artificial Sequence; identical to SEQ ID NO: 430
  Synthetic Construct

SEQ ID NO: 847 - RNA, 227 nt, Artificial Sequence; identical to SEQ ID NO: 431
  Synthetic Construct

SEQ ID NO: 848 - RNA, 227 nt, Artificial Sequence; identical to SEQ ID NO: 432
  Synthetic Construct

SEQ ID NO: 849 - RNA, 223 nt, Artificial Sequence
  Synthetic Construct
ccaguggggcacuuuuuauguguucuucugcugguuaaucucggcgcucaagaguuguauccacggguaguuuccaacuccrgcuggugugagaggagccguuccacgcaggacgcguacuagaagaagccgaagcaacuucaggucgccgcgcacgaacuggcgcucgaucuccgccucgcucgaguagguuuccccggggaucccucuucgagcccucgcc

SEQ ID NO: 850 - RNA, 225 nt, Artificial Sequence; identical to SEQ ID NO: 434
  Synthetic Construct

SEQ ID NO: 851 - RNA, 217 nt, Artificial Sequence
  Synthetic Construct
cuuaccuguauaguggcaagggcucgaagagguauccccggggaaaccuacucgagcgaggcggagaucgagcgccagggcgugcgcggcgaccugaaguugcucggcuucuucuuguucgcgcuggugaacgcuccuucuucacaccagccgaguuggaaacuacccgguggauacaacucuugagcgccgagcuuaaccugcuagaucggaggag

SEQ ID NO: 852 - RNA, 133 nt, Artificial Sequence
  Synthetic Construct
rgcacuycycyucygcccaycyuyuucuuguccrgcgccycrrgugaayccggaacccgaaaggruccggayccuuucggguuccggruucacyygrggcgcyggacuaaagaaraagrugggcrgargrgac

SEQ ID NO: 853 - RNA, 220 nt, Artificial Sequence; identical to SEQ ID NO: 437
  Synthetic Construct

SEQ ID NO: 854 - RNA, 220 nt, Artificial Sequence; identical to SEQ ID NO: 438
  Synthetic Construct

SEQ ID NO: 855 - RNA, 219 nt, Artificial Sequence; identical to SEQ ID NO: 439
  Synthetic Construct

SEQ ID NO: 856 - RNA, 218 nt, Artificial Sequence; identical to SEQ ID NO: 440
  Synthetic Construct

SEQ ID NO: 857 - RNA, 217 nt, Artificial Sequence; identical to SEQ ID NO: 441
  Synthetic Construct

SEQ ID NO: 858 - RNA, 217 nt, Artificial Sequence; identical to SEQ ID NO: 442
  Synthetic Construct

SEQ ID NO: 859 - RNA, 217 nt, Artificial Sequence; identical to SEQ ID NO: 443
  Synthetic Construct

SEQ ID NO: 860 - RNA, 217 nt, Artificial Sequence; identical to SEQ ID NO: 444
  Synthetic Construct

SEQ ID NO: 861 - RNA, 217 nt, Artificial Sequence; identical to SEQ ID NO: 445
  Synthetic Construct

SEQ ID NO: 862 - RNA, 216 nt, Artificial Sequence; identical to SEQ ID NO: 446
  Synthetic Construct

SEQ ID NO: 863 - RNA, 215 nt, Artificial Sequence; identical to SEQ ID NO: 447
  Synthetic Construct

SEQ ID NO: 864 - RNA, 214 nt, Artificial Sequence; identical to SEQ ID NO: 448
  Synthetic Construct

SEQ ID NO: 865 - RNA, 213 nt, Artificial Sequence; identical to SEQ ID NO: 449
  Synthetic Construct

SEQ ID NO: 866 - RNA, 208 nt, Artificial Sequence
  Synthetic Construct
agacccuucgucgacgacracaggugaguuccuucuucuccucguuucuuuaucuaucugcguuggcggggcgaaccacaggaaccgcacgguguuuaccgggaacaccuauuguguuuuacccugggaacccacccgccgcugcgucaaagaaaaagagcggggagaacagggggcuccacccggaguccgcucgacuagcggacaa

SEQ ID NO: 867 - RNA, 211 nt, Artificial Sequence; identical to SEQ ID NO: 451
  Synthetic Construct

SEQ ID NO: 868 - RNA, 208 nt, Artificial Sequence; identical to SEQ ID NO: 452
  Synthetic Construct

SEQ ID NO: 869 - RNA, 206 nt, Artificial Sequence; identical to SEQ ID NO: 453
  Synthetic Construct

SEQ ID NO: 870 - RNA, 205 nt, Artificial Sequence; identical to SEQ ID NO: 454
  Synthetic Construct

SEQ ID NO: 871 - RNA, 205 nt, Artificial Sequence; identical to SEQ ID NO: 455
  Synthetic Construct

SEQ ID NO: 872 - RNA, 204 nt, Artificial Sequence; identical to SEQ ID NO: 456
  Synthetic Construct

SEQ ID NO: 873 - RNA, 201 nt, Artificial Sequence; identical to SEQ ID NO: 457
  Synthetic Construct

SEQ ID NO: 874 - RNA, 197 nt, Artificial Sequence
  Synthetic Construct
gggauccccggggaaaccuggagcgaacuggcaaaaaggcgcgguggggagugcccagcggccgacaggaguaauuccugcagaaacaggguuuucacccuuccuuucuucggguuuccuuccucgcgcccgcagguccaccccucgcccccuuugcgcugucgcuucggcaacuacccgguggaacaacugaagcu

SEQ ID NO: 875 - RNA, 197 nt, Artificial Sequence; identical to SEQ ID NO: 459
  Synthetic Construct

SEQ ID NO: 876 - RNA, 180 nt, Artificial Sequence; identical to SEQ ID NO: 460
  Synthetic Construct

SEQ ID NO: 877 - RNA, 178 nt, Artificial Sequence; identical to SEQ ID NO: 461
  Synthetic Construct

SEQ ID NO: 878 - RNA, 145 nt, Artificial Sequence
  Synthetic Construct
cuggggaaaucuacagggcaccccaaaaacuacugcaggagaggccgcuugagggauccccgggggaauucgccgcgccaaucgaauucccgcggccaaucugggaagggagcguaccuggguagaucgugcgcguuggaggaga

SEQ ID NO: 879 - RNA, 136 nt, Artificial Sequence
  Synthetic Construct
cuggggaaaucuacagggcaccccaaaacuacugcaggagaggccgcuugaggguccccggggaaaccucaagcgaaucugggaagggagcguacyuggguagaucgugcugcguuggaggagaucyaucgucgcu

SEQ ID NO: 880 - RNA, 143 nt, Artificial Sequence
  Synthetic Construct
aagaggucggcaccugaugucgguguccugaugaagauccaugacaggaucgaaaccucuuccaguuucggcuugugcgggaguaaagcuuuygcucucuccacagccucaucaggaaacccacuucaggucucgacuggaag

SEQ ID NO: 881 - RNA, 143 nt, Artificial Sequence; identical to SEQ ID NO: 465
  Synthetic Construct

SEQ ID NO: 882 - RNA, 97 nt, Artificial Sequence; identical to SEQ ID NO: 466
  Synthetic Construct

SEQ ID NO: 883 - RNA, 65 nt, Artificial Sequence; identical to SEQ ID NO: 467
  Synthetic Construct

SEQ ID NO: 884 - RNA, 41 nt, Pospiviroid
  Potato spindle tuber viroid
cggaacuaaacucgugguuccugugguucacaccugaccuc

SEQ ID NO: 885 - RNA, 46 nt, Pospiviroid
  Potato spindle tuber viroid
gcuucggggcgaggguguuuagcccuuggaaccgcaguugguuccu

SEQ ID NO: 886 - RNA, 32 nt, Pospiviroid
  Potato spindle tuber viroid
cugagcagaaaagaaaaaagaaggcggcucgg

SEQ ID NO: 887 - RNA, 28 nt, Pospiviroid
  Potato spindle tuber viroid
cgagaaccgcuuuuucucuaucuuacuu

SEQ ID NO: 888 - RNA, 47 nt, Pospiviroid
  Potato spindle tuber viroid
aggagcgcuucagggauccccggggaaaccuggagcgaacuggcaaa

SEQ ID NO: 889 - RNA, 46 nt, Pospiviroid
  Potato spindle tuber viroid
ugcgcugucgcuucggcuacuacccgguggaaacaacugaagcucc

SEQ ID NO: 890 - RNA, 28 nt, Pospiviroid
  Potato spindle tuber viroid
aaaggacgguggggagugcccagcggcc

SEQ ID NO: 891 - RNA, 28 nt, Pospiviroid
  Potato spindle tuber viroid
cgcccgcaggaccaccccucgcccccuu

SEQ ID NO: 892 - RNA, 31 nt, Pospiviroid
  Potato spindle tuber viroid
gacaggaguaauucccgccgaaacaggguuu

SEQ ID NO: 893 - RNA, 32 nt, Pospiviroid
  Potato spindle tuber viroid
ucacccuuccuuucuucggguguccuuccucg

SEQ ID NO: 894 - RNA, 213 nt, Elaviroid
  Eggplant latent viroid
cuccccauaggguggugugugccaccccugaugagaccgaaaggucgaaaugggguuucgccaugggucgggacuuuaaauucggaggauucguccuuuaaacguuccuccaagagucccuuccccaaacccuuacuuuguaagugugguucggcgaauguaccguuucguccuuucggacucaucagggaaaguacacacuuuccgacggug

SEQ ID NO: 895 - RNA, 120 nt, Elaviroid
  Eggplant latent viroid
gguucgucgacaccucucccccucccagguacuauccccuuucaaggauguguucccuaggaggguggguguaccucuuuuggauugcuccggccuuccaggagagauagaggacgaccu

SEQ ID NO: 896 - RNA, 34 nt, Pospiviroid
  Potato spindle tuber viroid
cggaacuaaacucgugguuccugugguucacacc

SEQ ID NO: 897 - RNA, 33 nt, Pospiviroid
  Potato spindle tuber viroid
gguguuuagcccuuggaaccgcaguugguuccu

SEQ ID NO: 898 - RNA, 329 nt, Artificial Sequence
  Synthetic Construct
acguaaaaguuuccucugguuuuagcuugucggaucaaccuaacuucaucucuuucuuugagccgguauucuuuaacauacuguucggccaaguacugcgaucgcguuaacgcuuuaucacgauaccuucuaccacauaucacuaacaacaucaacacucaucaucuagacucucgacgacauccacucgucacuacucucacacgaccgauuaacuccucauccacgcggccgcgagcggccgaacaguauguuaaagaauaccggcucaaagaaagagaugaaguuagguugauccgacaagcuaaaaccagaggaaacuuuuacgu

SEQ ID NO: 899 - RNA, 547 nt, Artificial Sequence
  Synthetic Construct
cuucggcuacuacccgguggaaacaacugaagcucccgagaaccgcuuuuucucuaucuuacuugcuucggggcgaggguguuuagcccuuggaaccgcaguugguuccucggaacuaaacucgugguuccugugguucacaccugaccuccugagcagaaaagaaaaaagaaggcggcucggaggagcgcuucagggauccccggggaaaccuggagacguaaaaguuuccucugguuuuagcuugucggaucaaccuaacuucaucucuuucuuugagccgguauucuuuaacauacuguucggccaaguacugcgaucgcguuaacgcuuuaucacgauaccuucuaccacauaucacuaacaacaucaacacucaucaucuagacucucgacgacauccacucgucacuacucucacacgaccgauuaacuccucauccacgcggccgcgagcggccgaacaguauguuaaagaauaccggcucaaagaaagagaugaaguuagguugauccgacaagcuaaaaccagaggaaacuuuuacgu

SEQ ID NO: 900 - RNA, 167 nt, Artificial Sequence
  Synthetic Construct
accccugaugagaccgaaaggucgaaaugggguuucgccaugggucgggacuuuaaauucggaggauucguccuuuaaacguuccuccaagagucccuuccccaaacccuuacuuuguaagugugguucggcgaauguaccguuucguccuuucggacucaucaggg

SEQ ID NO: 901 - RNA, 463 nt, Encephalomyocarditis virus
gagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaa

SEQ ID NO: 902 - RNA, 552 nt, Encephalomyocarditis virus
acguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaaua

SEQ ID NO: 903 - RNA, 150 nt, Zea mays
agcacaacauuucaaccagaaacacuagccgaagcaaauccauuccacaagcaccuggugggaucaucucaucaucagaaaccaagagagagauuccguguccgcuuguuguaguagauugugaggacugaggaccgagaagcagccaug

SEQ ID NO: 904 - RNA, 228 nt, Unknown
  Crucifer infecting tobamovirus
aucuaaguuagguuguaaacauauuagagacguuguucauuuggaagaguuacgcgagucuuugugugauguagcuaguaaccuaaauaauugugcguauuuuucacaguuagaugaggccguugccgagguucauaagaccgcgguaggcgguucguuugcuuuuuguaguauaauuaaauauuugucagauaagagauuguuuagagauuuguucuuuguuugaua

SEQ ID NO: 905 - DNA, 143 bp, Tobacco etch virus
aaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagca

SEQ ID NO: 906 - RNA, 101 nt, Hibiscus chlorotic ringspot virus
uagcuuauuuggguucuuuaucacuguccugauuaaggcugaaucagaacaccaucacuuccacucacacgacaguaguaagacacagaacauaguaguua

SEQ ID NO: 907 - DNA, 278 bp, Classical swine fever virus
gagggactagccgtagtggcgagctccctgggtggtctaagtcctgagtacaggacagtcgtcagtagttcgacgtgagcactagcccacctcgagatgctacgtggacgagggcatgcccaagacacaccttaaccctagcgggggtcgctagggtgaaatcacattatgtgatgggggtacgacctgatagggtgctgcagaggcccactatcaggctagtataaaaaatctctgctgtacatggcacatggagttgaatcattttgaattattat

SEQ ID NO: 908 - DNA, 208 bp, Unknown
  Coxsackievirus B3 virus
tgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagattgttaccatatagctattggattggccatc

SEQ ID NO: 909 - DNA, 738 bp, Hepatitis A virus
tccatggtgaggggacttgatacctcaccgccgtttgcctaggctataggctaaattttccctttcccttttccctttcctattccctttgttttgcttgtaaatattaattcctgcaggttcagggttcttaaatctgtttctctataagaacactcatttttcacgctttctgtcttctttcttccagggctctccccttgccctaggctctggccgttgcgcccggcggggtcaactccatgattagcatggagctgtaggagtctaaattggggacacagatgtttggaacgtcaccttgcagtgttaacttggctttcatgaatctctttgatcttccacaaggggtaggctacgggtgaaacctcttaggctaatacttctatgaagagatgccttggatagggtaacagcggcggatattggtgagttgttaagacaaaaaccattcaacgccggaggactgactctcatccagtggatgcattgagtggattgactgtcagggctgtctttaggcttaattccagacctctctgtgcttagggcaaacatcatttggccttaaatgggattctgtgagaggggatccctccattgacagctggactgttctttggggccttatgtggtgtttgcctctgaggtactcaggggcatttaggtttttcctcattcttaaataataatgaacatgtctagacaaggtattttccagactgttgggagtggtct

SEQ ID NO: 910 - DNA, 423 bp, Unknown
  human rhinovirus human T-lymphotrophic virus
ggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctacgtctttgtttcgttttctgttctgcgccgttacagatcgaaagttccacccctttccctttcattcacgactgactgccggcttggcccacggccaagtaccggcaactctgctggctcggagccagcgacagcccattctatagcactctccaggagagaaatttagtacacagttgggggctcgtccgggat

SEQ ID NO: 911 - DNA, 301 bp, Unknown
  Polyoma virus
gctgctgctactggattttcagtagctgaaattgctgctggagaggccgctgctgcaattgaagtgcaacttgcatctgttgctactgttgaaggcctaacaacctctgaggcaattgctgctataggcctcactccacaggcctatgctgtgatatctggggctcctgctgctatagctggatttgcagctttactgcaaactgtgactggtgtgagcgctgttgctcaagtggggtatagattttttagtgactgggatcacaaagtttctactgttggtttatatcaacaaccaggaa

SEQ ID NO: 912 - DNA, 493 bp, Artificial Sequence
  Synthetic Construct
gggttgttgtgggttgaacccgtcccaaccatcaactcgctagccaaacacacgcttaggggccaaagcagtgctataatatgagtggtggcgctattatatatagcgtcagagaacttagatctgatattctgatgaagaaaaatgactttactgactacgaaagaagaagaaaggagctatagagagagagaaaaagaggggtcgtgtagtgcttaaactgtacatgaacagcagtagtgttacagaagctaaactcaaccagagctccaccaaagacaaagagggtctacttccatcaccgtcttgctcggtcacttggagctctgtccataaattaaacccatcttggatcccaaggttcgtggcatatctgtaggcatctaccccgtcttcgtcgtccgctcctcactagctaccaagaggtcgccattattgccaacatagagtgtacgtggatgtctatatatatgcctacttgcacccatatggc

SEQ ID NO: 913 - RNA, 21 nt, Artificial Sequence
  Synthetic Construct
uggacagagcuccaagugacc

SEQ ID NO: 914 - RNA, 20 nt, Artificial Sequence
  Synthetic Construct
uaauuucuacucuuguagau

SEQ ID NO: 915 - RNA, 447 nt, Artificial Sequence
  Synthetic Construct
ccccauaggguggugugugccaccccugaugagaccgaaaggucgaaaugggguuucgccaugggucgggacuuuaaauucggaggauucguccuuuaaacguuccuccaagagucccuuccccaaacccuuacuuuguaagugugguucggcgaauguaccguuucguccuuucggacucaucagggaaaguacacacuuuccgacgguggguucgucgacaccucucccccucccagguacuauccccuuuaauuucuacucuuguagauuggacagagcuccaagugaccuaauuucuacucuuguagauucaaggauguguucccuaggaggguggguguaccucuuuuggauugcuccggccuuccaggagagauagaggacgaccucuccccauaggguggugugugccaccccugaugagaccgaaaggucgaaaugggg

SEQ ID NO: 916 - DNA, 1107 bp, Artificial Sequence
  Synthetic Construct
aacacacgcttaggggccaaagcagtgctataatatgagtggtggcgctattatatatagcgtcagagaacttagatctgatattctgatgaagaaaaaatgactttactgactacgaaagaagaagaaaggagctatagagagagagaaaaagaggggtcgtgtagtgcttaaactgtacatgaacagcagtagtgttacagaagctaaactcaaccagagctccaccaaagacaaagagggtctacttccatcaccgtcttgctcggtcacttggagctctgtccataaattaaacccatcttggatcccaaggttcgtggcatatctgtaggcatctaccccgtcttcgtcgtccgctcctcactagctaccaagaggtcgccattattgccaacatagagtgtacgtggatgtctatatatatgcctacttgcacccatatggcataggcgttcgatccccttagcgcggaggagagctcctccggttcttctctacccttcgcatggaagttcttgcattgcttcgttgcttctctagtttcttccttctacgtctttccagcatacgcatgcccctcgtccgccggttcacgaggcatcgtctgatgatcagtagataataagcaatataatactgatctagaatcgagttgttgtactcttcgcagataggttcgttccttcacatagaagcgagtacagactacagaccacacagtatcagctggcacaaaacgaaaatggttacttgcaaattgcatgcacgagctagaattatattcttctaatcttcttcgttgactttctggcttcagcaggcgcgtgatggcgctttcagcttcccgagttcagcaggcggaagaactgctgcaacgacctgctgagaggcagctgatgaggagccagcttgctgcagccgccaggagcatcaactggagctacgccctcttctggtccatttcagacactcaaccagggtaaattaaagtcttattaattatgtacagttgatatttggttggcagttttctgtagttttgctgaatgatgatcatcgtcgcatcatttgcctgagcttttatagatcccaggctcccagcgc

SEQ ID NO: 917 - DNA, 23 bp, Artificial Sequence
  Synthetic Construct
aacacacgcttaggggccaaagc

SEQ ID NO: 918 - DNA, 23 bp, Artificial Sequence
  Synthetic Construct
gcgctgggagcctgggatctata

SEQ ID NO: 919 - DNA, 408 bp, Artificial Sequence
  Synthetic Construct
ggctcctgacaagaaaagaaaaaagaaggcggctcggaggagcgcttcagggatccccggggaaacctggagcgaactggcaaaaaaggacggtggggagtgcccagcggccgacaggagtaattcccgccgaaacagggttttcacccttcctcttcagttttctgtcaaaccacgcccgcaggaccacccctcgccccctttgcgctgtcgcttcggctactacccggtggaaacaactgaagctcccgagaaccgctttttctctatcttacttgctccggggcgagggtgtttagcccttggaaccgcagttggttcctcggaactaaactcgtggttcctgtggttcacacctgacctcctgacaagaaaagaaaaaagaaggcggctcggaggagcgcttca

SEQ ID NO: 920 - DNA, 430 bp, Artificial Sequence
  Synthetic Construct
ggaggagcgcttcagggatccccggggaaacctggagcgaactggcaaaaaaggacggtggggagtgcccagcggccgacaggagtaattcccgccgaaacagggttttcacccttcctttcttcgggtgtccttcctcgcgcccgcaggaccacccctcgccccctttgcgctgtcgcttcggctactacccggtggaaacaactgaagctcccgagaaccgctttttctctatcttacttgctccggggcgagggtgtttagcccttggaaccgccagttggttcctcggaactaaactcggcttcagttttctgtcaaaccatggttcctgtggttcacacctgacctcctgacaagaaaagaaaaaagaaggcggctcggaggagcgcttcagggatccccggggaaacctggagcgaactggctc

SEQ ID NO: 921 - DNA, 430 bp, Artificial Sequence
  Synthetic Construct
ggaggagcgcttcagggatccccggggaaaccgcttcagttttctgtcaaaccatggagcgaactggcaaaaaaggacggtggggagtgcccagcggccgacaggagtaattcccgccgaaacagggttttcacccttcctttcttcgggtgtccttcctcgcgcccgcaggaccacccctcgccccctttgcgctgtcgcttcgcgctactacccggtggaaacaactgaagctcccgagaaccgctttttctctatcttacttgctccggggcgagggtgtttagcccttggaaccgcagttggttcctcggaactaaactcgtggttcctgtggttcacacctgacctcctgacaagaaaagaaaaaagaaggcggctcggaggagcgcttcagggatccccggggaaacctggagcgaactggctc

SEQ ID NO: 922 - DNA, 405 bp, Artificial Sequence
  Synthetic Construct
ggaggagcgcttcagggatccccggggaaacctggagcgaactggcaaaaaaggacggtggggagtgcccagcggccgacaggagtaattcccgccgaaacagggttttcacccttcctttcttcgggtgtccttcctcgcgcccgcaggaccacccctcgccccctttgcgctgtcgcttcggctactacccggtggaaacaactgaagctcccgagaaccgctttttctctatcttacttgctccggggcgagggtgtttagcccttggaaccgcagttggttcctcggaactaaactcgtggttcctgtggttcacacctgacctcctgacaagaaaagaaaaaagaaggcggctcggaggagcgcttcagggatccccggggaaacctggagcgaactggc

SEQ ID NO: 923 - DNA, 426 bp, Artificial Sequence
  Synthetic Construct
aggagcgcttcagggatccccggggaaacctggagcgaactggcaaaaaaggacggtggggagtgcccagcggccgacaggagtaattcccgccgaaacagggttttcacccttcctttcttcgggtgtccttcctcgcgcccgcaggaccacccctcgccccctttgcgctgtcgcttcggctactacccggtggaaacaactgaagctcccgagaaccgctttttctctatcttacttgctccggggcgagggtgtttagcccttggaaccgccagttggttcctcggaactaaactcggcttcagttttctgtcaaaccatggttcctgtggttcacacctgacctcctgacaagaaaagaaaaaagaaggcggctcggaggagcgcttcagggatccccggggaaacctggagcgaactggc

SEQ ID NO: 924 - DNA, 426 bp, Artificial Sequence
  Synthetic Construct
aggagcgcttcagggatccccggggaaaccgcttcagttttctgtcaaaccatggagcgaactggcaaaaaaggacggtggggagtgcccagcggccgacaggagtaattcccgccgaaacagggttttcacccttcctttcttcgggtgtccttcctcgcgcccgcaggaccacccctcgccccctttgcgctgtcgcttcgcgctactacccggtggaaacaactgaagctcccgagaaccgctttttctctatcttacttgctccggggcgagggtgtttagcccttggaaccgcagttggttcctcggaactaaactcgtggttcctgtggttcacacctgacctcctgacaagaaaagaaaaaagaaggcggctcggaggagcgcttcagggatccccggggaaacctggagcgaactggc

(19) United States
(12) Patent Application Publication
(10) Pub. No.: US 2024/0254497 A1
(43) Pub. Date: Aug. 1, 2024

(54) VIROID-DERIVED POLYNUCLEOTIDES FOR MODIFICATION OF PLANTS

(71) Applicant: FLAGSHIP PIONEERING, INC., Cambridge, MA (US)

(72) Inventors: Yajie Niu, Lexington, MA (US); Swetha Srinivasa Murali, Somerville, MA (US); Michka Gabrielle Sharpe, Cambridge, MA (US); Barry Andrew Martin, Newport, RI (US)

(21) Appl. No.: 18/005,907

(22) PCT Filed: Jul. 20, 2021
(86) PCT No.: PCT/US2021/042414

§ 371 Date: Jan. 18, 2023

Related U.S. Application Data
(60) Provisional application No. 63/054,101, filed on Jul. 20, 2020.

(51) Int. Cl. C12N 15/82

(52) U.S. Cl. CPC C12N 15/8202

(57) ABSTRACT

Disclosed herein are viroid-derived polynucleotides for the modification of plants and methods of using such polynucleotides in a variety of agricultural and commercial methods. Specifically, the disclosure provides a method of delivering to a plant a composition comprising a recombinant polynucleotide comprising: (i) a single-stranded RNA (ssRNA) viroid sequence and (ii) a heterologous RNA sequence comprising or encoding an effector, wherein the effector has a biological effect on the plant, and wherein the viroid is potato spindle tuber viroid (PSTVd) or eggplant latent viroid (ELVd).

### SEQUENCE LISTING

The instant application contains a Sequence Listing which has been submitted electronically in ASCII format and is hereby incorporated by reference in its entirety. The ASCII copy, created on Jul. 19, 2021, is named 51484-002WO2_Sequence_Listing_7_19_21_ST25 and is 528,405 bytes in size.

### FIELD OF THE INVENTION

Provided herein are plant-modifying polynucleotides for use in a variety of agricultural and commercial applications.

### BACKGROUND

Plant viroids are circular, single-stranded RNAs capable of invading plants. There is need in the art for plant-modifying polynucleotides (e.g., polynucleotides derived from viroids) for use in a variety of agricultural and commercial applications.

### SUMMARY OF THE INVENTION

In one aspect, disclosed herein is a method of delivering an effector to a eukaryote, comprising providing to the eukaryote a composition comprising a recombinant polynucleotide comprising: (i) a single-stranded RNA (ssRNA) viroid sequence and (ii) a heterologous RNA sequence comprising or encoding an effector, whereby the effector comprised by or encoded by the heterologous RNA sequence is delivered to the eukaryote.

In some embodiments, the eukaryote is a plant, a fungus, or an animal.

In some embodiments, the composition is provided to a plant, plant tissue, or plant cell, or a processed product thereof, wherein the eukaryote consumes or contacts the plant, plant tissue, or plant cell, or processed product thereof, whereby the effector is delivered to the eukaryote.

In some embodiments, (a) the ssRNA viroid sequence is a viroid genome or a derivative thereof or (b) the ssRNA viroid sequence is a viroid genome fragment or a derivative thereof.

In some embodiments, the ssRNA viroid sequence is a sequence of a viroid from the family Pospiviroidae or Avsunviroidae. In some embodiments, the viroid is potato spindle tuber viroid (PSTVd) or eggplant latent viroid (ELVd).

In some embodiments, the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467.

In some embodiments, the ssRNA viroid sequence has at least 90% sequence identity to SEQ ID NO:51 or SEQ ID NO:50.

In some embodiments, the ssRNA viroid sequence does not contain a pathogenicity domain.

In some embodiments, the RNA sequence comprising or encoding the effector is not a viroid sequence and (a) has a biological effect on a plant or (b) has a biological effect on an animal or fungus that consumes or contacts the plant.

In some embodiments, the effector comprises or is encoded by an ssRNA sequence.

In some embodiments, the effector is an siRNA.

In some embodiments, the heterologous RNA sequence comprises coding RNA, non-coding RNA, or both coding and non-coding RNA.

In some embodiments, the effector comprises coding RNA, non-coding RNA, or both coding and non-coding RNA.

In some embodiments, the effector comprises non-coding RNA comprising at least one regulatory RNA or at least one interfering RNA that regulates a target gene or its transcript in a target cell.

In some embodiments, the target cell is selected from the group consisting of a plant cell, an animal cell, and a fungal cell.

In some embodiments, the effector modifies a trait, phenotype, or genotype in the target cell. In some embodiments, modifying comprises reducing expression of the target gene. In some embodiments, modifying comprises increasing expression of the target gene. In some embodiments, modifying comprises (a) editing the target gene or (b) regulating the target gene.

In some embodiments, the ssRNA viroid sequence effects one or more results selected from the group consisting of entry into a tissue or cell of the eukaryote; transmission through a tissue or cell or subcellular component of the eukaryote; replication in a tissue or cell of the eukaryote; targeting to a tissue or cell of the eukaryote; and binding to a factor in a tissue or cell of the eukaryote.

In some embodiments, the recombinant polynucleotide lacks free ends and/or is circular.

In some embodiments, the composition is topically delivered to a plant. In some embodiments, the topical delivery is spraying, leaf rubbing, soaking, coating, injecting, seed coating, or delivery through root uptake.

In another aspect, disclosed herein is a composition comprising a recombinant polynucleotide comprising: (a) a single-stranded RNA (ssRNA) viroid sequence that is a viroid genome or a derivative thereof or a viroid genome fragment or a derivative thereof, and (b) a heterologous RNA sequence that is not a viroid sequence and comprises or encodes an effector.

In some embodiments, the viroid genome is (a) a genome of a viroid from the family Pospiviroidae or Avsunviroidae, or (b) a genome of potato spindle tuber viroid (PSTVd) or eggplant latent viroid (ELVd).

In some embodiments, the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467.

In some embodiments, the ssRNA viroid sequence has at least 90% sequence identity to SEQ ID NO:51 or SEQ ID NO:50.

In some embodiments, the effector comprises non-coding RNA comprising at least one regulatory RNA or at least one interfering RNA or at least one guide RNA that regulates or modifies a target gene or its transcript in a target cell, wherein the target cell is a plant cell, an animal cell, or a fungal cell.

In some embodiments, the effector (a) modifies expression of a target gene in a eukaryotic cell; or (b) has a biological effect on a plant or on an animal or fungus that consumes or contacts the plant.

In some embodiments, the composition is (a) formulated for delivery to a plant or to the environment in which the plant grows; or (b) formulated for delivery to an animal or fungus.

In another aspect, disclosed herein is a composition comprising a recombinant polynucleotide comprising: (i) a single-stranded RNA (ssRNA) viroid sequence and (ii) a heterologous RNA sequence comprising or encoding an effector, the composition being formulated for topical delivery to a plant.

In some embodiments, the ssRNA viroid sequence is a viroid genome or a derivative thereof.

In some embodiments, the ssRNA viroid sequence is a viroid genome fragment or a derivative thereof.

In some embodiments, the recombinant polynucleotide encodes at least two ssRNA viroid sequences.

In some embodiments, the topical delivery is spraying, leaf rubbing, soaking, coating, injecting, seed coating, or delivery through root uptake.

In some embodiments, the composition further comprises an additional formulation component.

In some embodiments, the composition does not comprise an additional formulation component.

In some embodiments, the ssRNA viroid sequence comprises a sequence of at least 40 ribonucleotides which is at least 80% identical to a sequence, or fragment thereof, listed in Table 1. In some embodiments, the ssRNA viroid sequence has at least 90% identity to a sequence of Table 1. In some embodiments, the ssRNA viroid sequence has at least 95% identity to a sequence of Table 1. In some embodiments, the ssRNA viroid sequence has at least 98% identity to a sequence of Table 1. In some embodiments, the ssRNA viroid sequence has at least 99% identity to a sequence of Table 1.

In some embodiments, the sequence of Table 1 is SEQ ID NO: 50.

In some embodiments, the sequence of Table 1 is SEQ ID NO: 51.

In some embodiments, the viroid is from the family Pospiviroidae or Avsunviroidae.

In some embodiments, the viroid is eggplant latent viroid (ELVd), potato spindle tuber viroid (PSTVd), hop stunt viroid, coconut cadang-cadang viroid, apple scar skin viroid, Coleus blumei viroid 1, avocado sunblotch viroid, peach latent mosaic viroid, chrysanthemum chlorotic mottle viroid, or Dendrobium viroid.

In some embodiments, the viroid is ELVd. In some embodiments, the viroid is PSTVd.

In some embodiments, the ssRNA viroid sequence comprises a sequence that is at least 80% identical to a sequence listed in Table 2 or Table 3. In some embodiments, the ssRNA viroid sequence has at least 90% identity to a sequence of Table 2 or Table 3. In some embodiments, the ssRNA viroid sequence has at least 95% identity to a sequence of Table 2 or Table 3. In some embodiments, the ssRNA viroid sequence has at least 98% identity to a sequence of Table 2 or Table 3. In some embodiments, the ssRNA viroid sequence has at least 99% identity to a sequence of Table 2 or Table 3.

In some embodiments, each of the at least two ssRNA viroid sequences are at least 80% identical to a sequence listed in Table 2 or Table 3.

In some embodiments, the recombinant polynucleotide encodes a sequence that is at least 80% identical to SEQ ID NO: 884 and encodes a sequence that is at least 80% identical to SEQ ID NO: 885.

In some embodiments, the recombinant polynucleotide encodes a sequence that is at least 80% identical to SEQ ID NO: 886 and encodes a sequence that is at least 80% identical to SEQ ID NO: 887.

In some embodiments, the recombinant polynucleotide encodes a sequence that is at least 80% identical to SEQ ID NO: 888 and encodes a sequence that is at least 80% identical to SEQ ID NO: 889.

In some embodiments, the recombinant polynucleotide encodes a sequence that is at least 80% identical to SEQ ID NO: 890 and encodes a sequence that is at least 80% identical to SEQ ID NO: 891.

In some embodiments, the recombinant polynucleotide encodes a sequence that is at least 80% identical to SEQ ID NO: 892 and encodes a sequence that is at least 80% identical to SEQ ID NO: 893.

In some embodiments, the recombinant polynucleotide comprises 3, 4, 5, 6, 7, 8, 9, 10, or more than 10 ssRNA viroid sequences that are at least 80% identical to a sequence listed in Table 2 or Table 3.

In some embodiments, the ssRNA viroid sequence comprises, in secondary structure, one or more of a replication motif, a transmission motif, a targeting motif, or a binding motif.

In some embodiments, the ssRNA viroid sequence does not contain a pathogenicity domain.

In some embodiments, the ssRNA viroid sequence comprises an internal loop, a stem-loop, a bulge loop, or a pseudoknot.

In some embodiments, the ssRNA viroid sequence comprises a replication domain, a transmission domain, a targeting domain, or a binding domain. In some embodiments, the transmission domain is a tissue transmission domain, a cell-cell transmission domain, or a subcellular transition domain. In some embodiments, the targeting domain is a tissue targeting domain, a cell targeting domain, or a subcellular targeting domain. In some embodiments, the targeting domain binds to a host cell. In some embodiments, the targeting domain is a nuclear targeting sequence or a nuclear exclusion sequence. In some embodiments, the binding domain binds a molecular target in the plant. In some embodiments, the binding domain binds DICER.

In some embodiments, the RNA sequence comprising or encoding the effector is not a viroid sequence and has a biological effect on a plant.

In some embodiments, the effector comprises or is encoded by an ssRNA sequence.

In some embodiments, the effector comprises a coding sequence. In some embodiments, the coding sequence encodes a protein or a polypeptide.

In some embodiments, the effector is a regulatory RNA. In some embodiments, the regulatory RNA is a lncRNA, circRNA, tRF, tRNA, rRNA, snRNA, snoRNA, or piRNA. In some embodiments, the effector is an interfering RNA. In some embodiments, the effector is a dsRNA or a hpRNA. In some embodiments, the effector is a microRNA (miRNA) or a pre-miRNA. In some embodiments, the effector is a phasiRNA.

In some embodiments, the effector is a hcsiRNA. In some embodiments, the effector is a natsiRNA. In some embodiments, the effector is a guide RNA.

In some embodiments, the effector binds a target host cell factor. In some embodiments, the target host cell factor is a nucleic acid, a protein, a DNA, or an RNA.

In some embodiments, the recombinant polynucleotide further comprises an internal ribosome entry site (IRES), a 5′ homology arm, a 3′ homology arm, a polyadenylation sequence, a group I permuted intron-exon (PIE) sequence, an RNA cleavage site, a ribozyme, a DICER-binding sequence, an mRNA fragment comprising an intron, an exon, a combination of one or more introns and exons, an untranslated region (UTR), an enhancer region, a Kozak sequence, a start codon, or a linker. In some embodiments, the ribozyme is a hammerhead ribozyme, a riboswitch, or a twister/tornado. In some embodiments, the DICER-binding sequence flanks the effector.

In some embodiments, the recombinant polynucleotide comprises 2, 3, 4, 5, 6, 7, 8, 9, 10, or more than 10 additional heterologous sequence elements.

In some embodiments, the recombinant polynucleotide lacks free ends. In some embodiments, the recombinant polynucleotide is circular.

In some embodiments, the recombinant polynucleotide comprises at least one free end. In some embodiments, the recombinant polynucleotide is concatemeric. In some embodiments, the recombinant polynucleotide is linear.

In another aspect, disclosed herein is a cell comprising a composition of any of the above embodiments.

In some embodiments, the cell is a plant cell. In some embodiments, the plant cell is a monocot cell or a dicot cell. In some embodiments, the plant cell is a protoplast.

In some embodiments, the cell has been transiently transformed with the recombinant polynucleotide.

In some embodiments, the cell has been stably transformed with the recombinant polynucleotide.

In another aspect, disclosed herein is a composition according to any of the above embodiments, further comprising a plant cell.

In another aspect, disclosed herein is a liposome comprising a composition according to any of the above embodiments.

In another aspect, disclosed herein is a vesicle comprising a composition according to any of the above embodiments.

In another aspect, disclosed herein is a formulation comprising a composition according to any of the above embodiments.

In some embodiments, the formulation is a liquid, a gel, or a powder.

In some embodiments, the formulation is configured to be sprayed on plants, to be rubbed on leaves, to be coated on seeds, or to be delivered to roots.

In another aspect, disclosed herein is a method of delivering an effector to a plant, a plant tissue, or a plant cell, comprising providing to a plant, plant tissue, or plant cell a composition according to any one of the above embodiments, whereby the effector comprised by or encoded by the heterologous RNA sequence is delivered to the plant, plant tissue, or plant cell.

In some embodiments, the plant is a monocot or a dicot.

In some embodiments, the plant cell is a protoplast.

In some embodiments, providing the composition to the plant, plant tissue, or plant cell comprises delivering the composition to a leaf, root, stem, flower, seed, xylem, phloem, apoplast, symplast, meristem, fruit, embryo, microspore, pollen, pollen tube, ovary, ovule, or explant for transformation of the plant.

In some embodiments, the fruit is a pre-harvest fruit. In some embodiments, the fruit is a post-harvest fruit.

In another aspect, disclosed herein is a method of modifying a trait, phenotype, or genotype in a plant cell, comprising providing to the plant cell a composition according to any of the above embodiments.

In some embodiments, modifying comprises expressing in the plant a heterologous protein encoded by the RNA sequence comprising or encoding an effector.

In some embodiments, modifying comprises reducing expression of a target gene of the plant.

In some embodiments, modifying comprises increasing expression of a target gene of the plant.

In some embodiments, modifying comprises editing a target gene of the plant.

In some embodiments, modifying comprises regulating a target gene in the plant.

In some embodiments, the ssRNA viroid sequence effects one or more results selected from the group consisting of entry into a tissue or cell of the plant; transmission through a tissue or cell or subcellular component of the plant; replication in a tissue or cell of the plant; targeting to a tissue or cell of the plant; and binding to a factor in a tissue or cell of the plant.

In another aspect, disclosed herein is a composition comprising a recombinant polynucleotide comprising: (i) a single-stranded RNA (ssRNA) viroid sequence and (ii) a heterologous RNA sequence comprising or encoding an effector, wherein the ssRNA viroid sequence does not include a chloroplast localization sequence.

Other features and advantages of the invention will be apparent from the following Detailed Description and the Claims.

### Definitions

As used herein, the term “internal ribosome entry site” or “IRES” refers to a sequence (e.g., an RNA sequence) capable of recruiting a ribosome and translation machinery to initiate translation from an RNA sequence. An IRES element is generally between 100-800 nucleotides. In embodiments, the efficiency or effectiveness of an IRES in the composition and methods described herein is tested, e.g., by introducing the IRES into a circular RNA expression vector and assaying for levels of expression of a downstream cistronic protein such as firefly luciferase using enzymatic reactions, or fluorescent readouts using reporters such as green fluorescent protein (GFP). An appropriate IRES can be obtained from plant and plant viral IRES sequences such as encephalomyocarditis virus IRES (ECMV), maize hsp101 IRES 5′UTR, crucifer infecting tobamovirus crTMV CR-CP 148 IRES, tobacco etch virus (TEV) IRES 5′UTR and hibiscus chlorotic ringspot virus (HCRSV) IRES. In addition, in embodiments, an IRES sequence is derived from non-plant eukaryotic virus sequences that include but are not limited to: acute bee paralysis virus (ABPV), classical swine fever virus (CSFV), coxsackievirus B3 virus (CVB3), encephalomyocarditis virus (ECMV), enterovirus 71 (E71), hepatitis A virus (HAV), human rhinovirus (HRV2), human rhinovirus (HRV2), human lymphotropic virus (HTLV) and polyoma virus (PV). Examples of IRES sequence useful in the compositions and methods described herein are shown in Table 4.

As used herein, the term “untreated” refers to an organism (e.g., a eukaryote, e.g., a plant, a fungus, or an animal) that has not been contacted with or delivered a recombinant polynucleotide (e.g., viroid-derived vector) described herein, including a separate organism that has not been delivered the recombinant polynucleotide, the same organism undergoing treatment assessed at a time point prior to delivery of the recombinant polynucleotide, or the same organism undergoing treatment assessed at an untreated part of the organism (that is, at an area of the organism not contacted with the recombinant polynucleotide).

As used herein, the term “effective amount,” “effective concentration,” or “concentration effective to” refers to an amount of a recombinant polynucleotide (e.g., viroid-derived vector) or a composition thereof, sufficient to effect the recited result or to reach a target level (e.g., a predetermined or threshold level) in or on a target organism.

As used herein, the term “topical delivery to a plant”, and variants thereof, refers to any method of delivering a composition (e.g. a recombinant polynucleotide described herein) to a plant that does not comprise transformation (e.g., does not comprise direct introduction of the composition to the cytoplasm of the cell, e.g., does not comprise Agobacterium-mediated transformation, viral vector-mediated transformation, electroporation, or use of a gene gun (biolistics)). Methods of topical delivery include, but are not limited to spraying, leaf rubbing, soaking (e.g., soaking of leaves, roots, stems, or other plant parts), coating (e.g., soaking of leaves, roots, stems, or other plant parts, e.g., coating using micro-particulates or nano-particulates), injection (e.g., injection into leaves, roots, stems, or other plant parts), seed coating, and delivery through root uptake (e.g., delivery in a hydroponic system or delivery in another growth medium, e.g., soil).

As used herein, the phrases “modulating a state of an organism”, “modulating a state of a cell”, and variants thereof refer to an observable change in a state (e.g., the transcriptome, proteome, epigenome, biological effect, or health or disease state) of the organism or cell (e.g., plant or plant cell; arthropod or arthropod cell; mollusk or mollusk cell; fungus or fungus cell; or nematode or nematode cell), as measured using techniques and methods known in the art for such a measurement, e.g., methods to measure the level or expression of a protein, a transcript, or an epigenetic mark, or to measure the increase or reduction of activity of a protein or biological pathway. In some embodiments, the modulation is transient, e.g., does not persist for the lifetime of the organism or cell. In other embodiments, the modulation persists for the lifetime of the organism or cell, but is not inherited by a progeny of the organism or cell. In still other embodiments, the modulation is inherited by a progeny of the organism or cell, e.g., a progeny produced by sexual reproduction, asexual reproduction, or cell division.

In some embodiments, modulating a state of an organism or a cell comprises modifying the organism or cell. As used herein, “modifying an organism”, “modifying a cell”, and variants thereof refer to changing one or more characteristics of a genome of the cell (e.g., a nuclear, mitochondrial, or plastid genome of the cell), e.g., altering the nucleotide sequence or the methylation status of one or more genetic sequences.

In some embodiments, modulating a state of the organism or cell (e.g., modifying the organism or cell) results in a change (e.g., an increase or decrease) of the state by at least 1% relative to a reference level (e.g., a level found in an organism or cell that is not subjected to the treatment or contacted with the composition), e.g., a change of at least 5%, 10%, 15%, 20%, 25%, 30%, 35%, 40%, 45%, 50%, 55%, 60%, 65%, 70%, 75%, 80%, 85%, 90%, 95%, 98% or more than 98% relative to a reference level. In some embodiments, modulating the state of the organism or cell (e.g., modifying the organism or cell) involves increasing a parameter (e.g., the level or expression of a protein, a transcript, or activity of a biological pathway) of the organism or cell, e.g., increasing the parameter by at least 1% relative to a reference level (e.g., at least 5%, 10%, 15%, 20%, 25%, 30%, 35%, 40%, 45%, 50%, 55%, 60%, 65%, 70%, 75%, 80%, 85%, 90%, 95% 98%, 99%, 100% or more than 100% relative to a reference level). In other embodiments, modulating the state of the organism or cell (e.g., modifying the organism or cell) involves decreasing a parameter (e.g., the level or expression of a protein, a transcript, or activity of a biological pathway) of the organism or cell, e.g., decreasing the parameter by at least 1% relative to a reference level (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to a reference level; e.g., up to 100% relative to a reference level). In some embodiments, properties that are modulated include, but are not limited to (a) one or more genetic or epigenetic characteristics of a nuclear or organellar genome or the organism or cell (e.g., altering the nucleotide sequence or the methylation status of one or more genetic sequences; increasing, decreasing, or otherwise altering gene expression; transiently or stably introducing into the organism or cell a heterologous nucleotide or polypeptide sequence); (b) one or more physiological or biochemical properties of the organism or cell (e.g., altering amino acid, lipid, carbohydrate, vitamin or pro-vitamin, or other nutritional content; altering response to biotic or abiotic stimuli); (c) one or more phenotypic properties of the organism or cell (e.g., flower or leaf appearance, branching or other architectural characteristics, fruit or seed number or size, appearance, or flavor of a plant or a plant part); (d) one or more agronomic or commercially important characteristics of an organism or cell (e.g., flowering time, nutrient use efficiency, water use efficiency, intrinsic yield; resistance of tissue to bruising, oxidation, or softening; resistance to diseases or pests; seed or fruit storage characteristics, or digestibility of a plant or a plant part); or any combination of these properties. In some embodiments, the modulation results in a desirable change or improvement of the organism or cell (e.g., a desirable change or improvement in a plant, a seed of the plant, or a product made from the plant. For example, in embodiments, the modification results in an increase in the fitness of the organism or cell, e.g., an increase in plant fitness. In other embodiments, the modification results in a decrease in the fitness of the organism or cell, e.g., a decrease in plant fitness, (e.g., plant death and/or a decrease in plant fecundity) or a decrease in fitness of a plant pest (e.g., death and/or decreased fecundity of the plant pest, e.g., arthropod, nematode, mollusk, or fungus).

As used herein, the term “effector” refers to a moiety that can be integrated into a recombinant polynucleotide (e.g., viroid-derived vector) and that is capable of modulating (e.g., modifying) a state of a plant or plant cell; an arthropod or an arthropod cell; a mollusk or a mollusk cell; a fungus or a fungus cell; or a nematode or a nematode cell. In embodiments, the effector comprises or is encoded by an RNA sequence, e.g., a single-stranded RNA (ssRNA) sequence. In embodiments, the effector comprises a coding sequence (e.g., a protein-coding sequence). In embodiments, the effector is, e.g., a regulatory RNA (e.g., a lncRNA, circRNA, tRF, tRNA, rRNA, snRNA, snoRNA, or a piRNA), an interfering RNA, a dsRNA, a microRNA (miRNA) or a pre-miRNA, a phasiRNA, a hcsiRNA, a natsiRNA, or a guide RNA. In embodiments, the effector binds a factor in the target host cell, e.g., binds a nucleic acid, a protein, a peptide, a DNA, an RNA, or a small molecule (e.g., a metabolite or ion).

As used herein, the term “heterologous”, when used to describe a first element in reference to a second element means that the first element and second element do not exist in nature disposed as described. For example, a heterologous nucleic acid molecule or sequence is a nucleic acid molecule or sequence that (a) is not native to a cell in which it is expressed, (b) is linked or fused to a nucleic acid molecule or sequence with which it is not linked to or fused to in nature, or with which it is not linked to or fused to in nature in the same way, (c) has been altered or mutated by the hand of man relative to its native state, or (d) has altered expression as compared to its native expression levels under similar conditions. For example, a heterologous RNA relative to a viroid RNA means the heterologous RNA does not exist as part of, or linked to, the viroid RNA in its naturally-occurring state. For example, a recombinant polynucleotide such as those provided by this disclosure can include genetic sequences of two or more different viroids, which genetic sequences are “heterologous” in that they would not naturally occur together. In some embodiments “heterologous” refers to a molecule; for example, a cargo or payload (e.g., a nucleic acid such as a protein-encoding RNA, an ssRNA, a regulatory RNA, an interfering RNA, or a guide RNA) or a structure (e.g., a plasmid or a gene-editing system) that is not found naturally in a plant viroid.

As used herein, “increase the fitness of a plant” refers to an increase in the fitness of the plant directly resulting from contact with a recombinant polynucleotide (e.g., viroid-derived vector) described herein and includes, for example, an improved yield, improved vigor of the plant, or improved quality or amount of a harvested product from the plant, an improvement in pre- or post-harvest traits deemed desirable for agriculture or horticulture (e.g., taste, appearance, shelf life), or for an improvement of traits that otherwise benefit humans (e.g., decreased allergen production). An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional plant-modifying agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. Yield can be expressed in terms of an amount by weight or volume of the plant or a product of the plant on some basis. The basis can be expressed in terms of time, growing area, weight of plants produced, or amount of a raw material used. An increase in the fitness of plant can also be measured in other ways, such as by an increase or improvement of the vigor rating, increase in the stand (the number of plants per unit of area), increase in plant height, increase in stalk circumference, increase in plant canopy, improvement in appearance (such as greener leaf color as measured visually), improvement in root rating, increase in seedling emergence, protein content, increase in leaf size, increase in leaf number, fewer dead basal leaves, increase in tiller strength, decrease in nutrient or fertilizer requirements, increase in seed germination, increase in tiller productivity, increase in flowering, increase in seed or grain maturation or seed maturity, less plant lodging, increased shoot growth, or any combination of these factors, by a measurable or noticeable amount over the same factor of the plant produced under the same conditions, but without the administration of the instant compositions or with application of conventional agricultural agents.

As used herein, “decrease the fitness of a plant” refers to a decrease in the fitness of the plant directly resulting from contact with a recombinant polynucleotide described herein and includes, for example, decreased survival (e.g., death) and/or decreased growth rate, tillering, plant biomass, pollen production, fecundity (e.g., seed yield), seed germination, or fruit yield of the plant compared to a plant grown under the same conditions, but without the application of the instant compositions or compared with application of conventional plant-modifying agents. For example, fitness can be decreased by at least about 0.5%, about 1%, about 2%, about 3%, about 4%, about 5%, about 10%, about 20%, about 30%, about 40%, about 50%, about 60%, about 70%, about 80%, about 90%, or about 100%.

As used herein, the term “formulated for delivery to a plant” refers to a recombinant polynucleotide (e.g., viroid-derived vector) composition that includes an active agent (e.g., a recombinant polynucleotide) and an additional formulation component, e.g., an agriculturally acceptable additional formulation component.

As used herein, an “agriculturally acceptable” formulation component is one that is suitable for use in agriculture, e.g., for use on plants. In certain embodiments, the additional formulation component does not have undue adverse side effects to the plants, the environment, or to humans or animals who consume the resulting agricultural products derived therefrom commensurate with a reasonable benefit/risk ratio.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. In embodiments, the plant or plant cell is haploid, diploid, triploid, tetraploid, pentaploid, hexaploid, or octoploid. In embodiments, a haploid plant or plant cell treated with a composition such as those described in this disclosure is further subjected to a haploid doubling treatment, resulting in a doubled-haploid plant or plant cell. Plant parts include differentiated and undifferentiated tissues including, but not limited to the following: roots, stems, shoots, leaves, pollen, seeds, fruit, harvested produce, tumor tissue, sap (e.g., xylem sap and phloem sap), and various forms of cells and culture (e.g., single cells, protoplasts, embryos, and callus tissue).

As used herein the term “percent identity” refers to percent (%) sequence identity with respect to a reference polynucleotide (e.g., ribonucleotide) or polypeptide sequence following alignment by standard techniques. Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software.

In some embodiments, the software is MUSCLE (Edgar, Nucleic Acids Res., 32(5): 1792-1797, 2004). Those skilled in the art can determine appropriate parameters for aligning sequences, including any algorithms needed to achieve maximal alignment over the full length of the sequences being compared.

For example, in embodiments, percent sequence identity values are generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction XN)

where X is the number of nucleotides or amino acids scored as identical matches by a sequence alignment program (e.g., BLAST) in that program's alignment of A and B, and where Y is the total number of nucleotides or amino acids in B.

### BRIEF DESCRIPTION OF THE DRAWINGS

FIG. 1 is a diagram showing the secondary structure of the RNA vector ELVd-hpRNA (SEQ ID NO: 3), which comprises an eggplant latent viroid (ELVd) sequence and a heterologous hairpin RNA sequence (hpRNA-SIPDS: indicated by box) targeting the Solanum lycopersicum phytoene desaturase (PDS) gene.

FIG. 2 is a diagram showing the secondary structure of the RNA vector ELVd-gRNA (SEQ ID NO: 5), which comprises an ELVd sequence (indicated by box with dashed lines) and a heterologous guide RNA sequence (gRNA-g12; indicated by box with solid lines) targeting the Zea mays glossy2 (gl2) gene.

FIG. 3 is a diagram showing the secondary structure of the RNA vector PSTVd-sRNA (SEQ ID NO: 10), which comprises a potato spindle tuber viroid (PTSVd) sequence (no color) and a heterologous small RNA sequence (sRNA-SIPDS: indicated by box) targeting the Solanum lycopersicum PDS gene.

FIG. 4A is a diagram showing the secondary structure of the RNA vector TL-R-amiR-PDS (SEQ ID NO: 14), which comprises the PSTVd left-terminal region (TL-R; indicated by box with dashed lines) and a heterologous pre-miRNA (amiR-PDS; indicated by box with solid lines) targeting the Solanum lycopersicum PDS gene.

FIG. 4B is a diagram showing the secondary structure of the RNA vector R-amiR-PDS (SEQ ID NO: 15), which comprises the PSTVd left-terminal and central conserved region (indicated by box with solid lines) and a heterologous pre-miRNA (amiR-PDS; indicated by box with dashed lines) targeting the Solanum lycopersicum PDS gene.

FIG. 5 is a diagram showing the secondary structure of the RNA vector PSTVd/TLR-circGORK (SEQ ID NO: 17), which comprises the terminal left region (TLR) ribonucleotides 331 to 347, covering loops 3 to 5, of PSTVd-RG1 (indicated by box) and a heterologous circular RNA derived from the intron segment flanking exons 2 and 3 of the Arabidopsis GATED OUTWARDLY-RECTIFYING K+ CHANNEL (GORK) gene (circGORK).

FIG. 6A is a diagram showing the secondary structure of the RNA vector CircRNA1 (SEQ ID NO: 20), which comprises a PSTVd right terminal domain containing transmission motifs (loop 26 and loop 27) and a heterologous intact Broccoli RNA aptamer sequence (indicated by box).

FIG. 6B is a diagram showing the secondary structure of the RNA vector CircRNA2 (SEQ ID NO: 23), which comprises a PSTVd right terminal domain containing transmission motifs (loop 26 and loop 27), a linker region, and a heterologous split Broccoli RNA aptamer sequence (indicated by box).

FIG. 7 is a diagram showing the secondary structure of a linear PSTVd-spinach fusion RNA vector (SEQ ID NO: 27), which comprises a PSTVd sequence in which the pathogenicity domain has been deleted and replaced with a heterologous Spinach RNA aptamer (indicated by box).

FIG. 8 is a diagram showing the secondary structure of the RNA vector ELVd-spinach (SEQ ID NO: 29), which comprises the ELVd complete genome, isolate 2 and a heterologous Spinach RNA aptamer (indicated by box).

FIG. 9 is a diagram showing the secondary structure of the RNA vector PSTVd/TR-amiR-PDS (SEQ ID NO: 32), which comprises the PSTVd right terminal domain containing transmission motifs (loop 26 and loop 27; indicated by box) and a heterologous pre-miRNA targeting the Solanum lycopersicum PDS gene.

FIG. 10 is a diagram showing the secondary structure of the circular RNA vector PSTVd/TL-Spinach (SEQ ID NO: 34), comprising the PSTVd left terminal domain (SEQ ID NO: 33; indicated by box); a heterologous Spinach RNA aptamer (SEQ ID NO: 26); and Loop 6 (L6) (U43/C318) (SEQ ID NO: 33) of PSTVd.

FIG. 11A is a pair of diagrams showing alternative predicted secondary structures of a circular RNA vector (SEQ ID NO: 35) comprising a PSTVd left terminal region (SEQ ID NO: 18; indicated by box in the left structure), a heterologous encephalomyocarditis virus internal ribosome entry site (EMCV IRES) (SEQ ID NO: 37); and nanoluciferase (SEQ ID NO: 39).

FIG. 11B is a diagram showing the secondary structure of a circular RNA vector (SEQ ID NO: 40) comprising a PSTVd left terminal region (SEQ ID NO: 18; indicated by box), a heterologous maize HSP101 internal ribosome entry site (MAIZE HSP101 IRES) (SEQ ID NO: 38); and nanoluciferase (SEQ ID NO: 39).

FIG. 12 is a pair of diagrams showing alternative predicted secondary structures of circular fusion RNA 3 (CircRNA3; SEQ ID NO: 43), which comprises a PSTVd viral trafficking motif sequence (SEQ ID NO: 42), a heterologous Spinach RNA aptamer sequence (SEQ ID NO: 26; indicated by box in the top structure); and a PSTVd replication motif sequence (SEQ ID NO: 41).

FIG. 13 is a diagram showing the secondary structure of the RNA vector PSTVd/TLR-spinach-TPMVd/TLR, which comprises the TLR (331 to 347 nts covering loops 3 to 5; indicated by lower left box) of PSTVd-RG1, the TLR (1 to 72 nts) of TPMVd isolate Mex8 (middle box), and a heterologous Spinach RNA aptamer sequence (upper box).

FIG. 14 is a diagram showing the secondary structure of circular fusion RNA 4 (CircRNA4; SEQ ID NO: 47), which comprises a PSTVd viral trafficking motif sequence (SEQ ID NO: 18) and an intact heterologous Broccoli RNA aptamer sequence (SEQ ID NO: 19; indicated by box).

FIG. 15 is a diagram showing the secondary structure of the RNA vector R-hpRNA-RPL7 (SEQ ID NO: 899), which comprises a replication domain of PSTVd (potato spindle tuber viroid), consisting of PSTVd TL-CCR (SEQ ID NO: 12) and heterologous hpRNA-RPL7 (SEQ ID NO: 898), a hairpin RNA targeting the Ribosomal Protein L7 gene of Leptinotarsa decemlineata.

FIG. 16 is a diagram showing the secondary structure of PSTVd.

FIG. 17 is a diagram showing the secondary structure of ELVd.

### DETAILED DESCRIPTION OF THE INVENTION

### Viroid-Derived Polynucleotides

Viroids are small, circular, single-stranded RNAs (ssRNAs) that lack a protein coating and are characterized by secondary structures including regions of intramolecular base pairing (stems) and non-paired loops and bulges. Viroids are capable of invading and replicating in plants, and can be virulent, mildly to moderately pathogenic, or commensal with the host plant. This disclosure provides recombinant polynucleotides (e.g., recombinant ssRNAs, e.g. recombinant ssRNA vectors) comprising one or more sequences of or derived from a viroid and one or more heterologous effector sequences that have a biological effect on an organism; compositions comprising such recombinant polynucleotides (e.g., compositions for topical application to a plant); and methods for modifying plants by delivery of such recombinant polynucleotides. Further provided are methods for modifying insects, mollusks, fungi, and nematodes by providing for consumption a plant comprising a recombinant polynucleotide such as those described in this disclosure.

Aspects of this disclosure are related to a composition comprising a recombinant polynucleotide comprising: ( ) a single-stranded RNA (ssRNA) viroid sequence and (ii) a heterologous RNA sequence comprising or encoding an effector. In some embodiments, the ssRNA viroid sequence is a viroid genome or a derivative thereof, e.g., a viroid genome or derivative thereof described in Section IA, Table 1, and/or Appendix 1 herein. In some embodiments, the ssRNA viroid sequence is a viroid genome fragment or a derivative thereof. For example, in embodiments, the ssRNA viroid sequence is a viroid genome or derivative thereof described in Section IB and Tables 2 and 3 herein, e.g., a functional domain of a viroid.

In some embodiments, the recombinant polynucleotide comprises at least two ssRNA viroid sequences, e.g., comprises 2, 3, 4, 5, 6, 7, 8, 9, 10, or more than 10 ssRNA viroid sequences. The two or more ssRNA sequences can occur contiguously or non-contiguously in the recombinant polynucleotide. The two or more ssRNA sequences can be derived from a single viroid (e.g., the recombinant polynucleotide includes two or more fragments or functional domains from a single viroid genome), or can be derived from more than one viroid (e.g., the recombinant polynucleotide encodes two or more viroid genomes or fragments or functional domains from two or more viroid genomes). In instances in which the ssRNA sequences are derived from a single viroid, sequences can be included in the recombinant polynucleotide in an order that corresponds to the order of the sequences in a wild-type version of the viroid, or can be rearranged. In some embodiments, the recombinant polynucleotide comprises more than one copy of a viroid sequence, e.g., comprises two, three, four, five, or more than five copies of such a sequence.

In some embodiments, the ssRNA viroid sequence comprises a loop, an internal loop, a stem-loop, a bulge loop, or a pseudoknot. In some embodiments, the ssRNA viroid sequence comprises a secondary structure element, e.g., a loop, internal loop, stem-loop, bulge loop, or pseudoknot, that is present in a wild-type version of the viroid from which the ssRNA viroid sequence is derived.

In embodiments, the ssRNA viroid sequence participates in, e.g., invasion of the recombinant polynucleotide into plant cells and/or replication of the recombinant polynucleotide in plant cells, thus delivering the effector to plant cells. In some embodiments, the ssRNA viroid sequence effects one or more of entry into a tissue or cell of the plant (e.g., entry into a leaf, root, or stem or a cell thereof); transmission to or through a tissue or cell or subcellular component of the plant; replication in a tissue or cell of the plant; targeting to a tissue or cell of the plant; and binding to a factor in a tissue or cell of the plant.

In some embodiments, the ssRNA viroid sequence comprises, in secondary structure, one or more of a replication motif, a transmission motif, a targeting motif, or a binding motif. In some embodiments, the ssRNA viroid sequence comprises one or more of a replication domain, a transmission domain, a targeting domain, or a binding domain.

In some embodiments, the transmission domain is a tissue transmission domain, a cell-cell transmission domain, or a subcellular transition domain.

In some embodiments, the targeting domain is a tissue targeting domain, a cell targeting domain, or a subcellular targeting domain. In some embodiments, the targeting domain binds to a host cell. In some embodiments, the targeting domain is a nuclear targeting sequence or a nuclear exclusion sequence. In some embodiments, the targeting domain is a plastid targeting sequence, e.g., a chloroplast targeting sequence.

In some embodiments, the binding domain binds a molecular target in the plant. In some embodiments, the binding domain binds DICER.

In some embodiments, the ssRNA viroid sequence does not contain a pathogenicity domain. In some embodiments, the ssRNA viroid sequence is not pathogenic, e.g., is not pathogenic to a plant to which the composition is delivered or is not pathogenic to any plant.

In some embodiments, the recombinant polynucleotide does not comprise an additional polynucleotide sequence for delivery to a cell, e.g., does not comprise a plasmid or a vector (e.g., a viral vector).

In some aspects, the disclosure provides a composition comprising a recombinant polynucleotide comprising (i) a single-stranded RNA (ssRNA) viroid sequence and (ii) a heterologous RNA sequence comprising or encoding an effector, wherein the ssRNA viroid sequence does not include a chloroplast localization sequence.

### Polynucleotides Comprising Viroid Genomes or Fragments Thereof.

In some embodiments, the ssRNA viroid sequence comprises a sequence of at least 15 ribonucleotides (e.g., at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 ribonucleotides or more than 300 ribonucleotides) which is at least 80% identical to a viroid genome sequence or a fragment thereof, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a viroid genome sequence or a fragment thereof.

In some embodiments, the viroid is from the family Pospiviroidae or Avsunviroidae. In some embodiments, the viroid is eggplant latent viroid (ELVd), potato spindle tuber viroid (PSTVd), hop stunt viroid, coconut cadang-cadang viroid, apple scar skin viroid, Coleus blumei viroid 1, avocado sunbiotch viroid, peach latent mosaic viroid, chrysanthemum chlorotic mottle viroid, or Dendrobium viroid. In some embodiments, the viroid is ELVd. In other embodiments, the viroid is PSTVd. In some embodiments, the viroid is a viroid provided in Table 1 and/or Appendix 1.

In some embodiments, the ssRNA viroid sequence comprises a sequence of at least 15 ribonucleotides (e.g., at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 ribonucleotides or more than 300 ribonucleotides) which is at least 80% identical to a sequence, or fragment thereof, listed in Table 1, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a sequence listed in Table 1. In some embodiments, the ssRNA viroid sequence has at least 90% identity to a sequence of Table 1. In some embodiments, the ssRNA viroid sequence has at least 95% identity to a sequence of Table 1. In some embodiments, the ssRNA viroid sequence has at least 98% identity to a sequence of Table 1. In some embodiments, the ssRNA viroid sequence has at least 99% identity to a sequence of Table 1. In some embodiments, the ssRNA viroid sequence comprises or consists of a sequence listed in Table 1.

In some embodiments, the ssRNA viroid sequence comprises a sequence of at least 40 ribonucleotides which is at least 80% identical to a sequence, or fragment thereof, listed in Table 1, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a sequence listed in Table 1.

In some embodiments, the ssRNA viroid sequence comprises a sequence of at least 100 ribonucleotides which is at least 80% identical to a sequence, or fragment thereof, listed in Table 1, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a sequence listed in Table 1.

In some embodiments, the ssRNA viroid sequence comprises a sequence of at least 15 ribonucleotides (e.g., at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 ribonucleotides or more than 300 ribonucleotides) which is at least 80% identical to a sequence of an eggplant latent viroid (ELVd), e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a sequence of an ELVd. In some embodiments, the ELVd sequence is SEQ ID NO: 50 (Table 1).

In some embodiments, the ssRNA viroid sequence comprises a sequence of at least 15 ribonucleotides (e.g., at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 ribonucleotides or more than 300 ribonucleotides) which is at least 80% identical to a sequence of a potato spindle tuber viroid (PSTVd), e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a sequence of a PSTVd. In some embodiments, the PSTVd sequence is SEQ ID NO: 51 (Table 1).

TABLE 1
Plant viroid sequences

| Name | SEQ ID NO: |
|---|---|
| Elaviroid AJ536613.1 Eggplant latent viroid complete genome, isolate 2 | SEQ ID NO: 50 |
| Pospiviroid U23058.1 Potato spindle tuber viroid (PSTVd) strain RG 1, complete | SEQ ID NO: 51 |
| genome | |
| Pospiviroid AY671952.1 Citrus exocortis viroid isolate D-104, complete genome | SEQ ID NO: 52 |
| Pospiviroid DQ076250.1 Columnea latent viroid clone PC-2-Pa54 plus 88, | SEQ ID NO: 53 |
| complete genome | |
| Pospiviroid AY671954.1 Citrus exocortis viroid isolate D-76, complete genome | SEQ ID NO: 54 |
| Pelamoviroid MK947213.1 Apple hammerhead viroid isolate Gala, complete | SEQ ID NO: 55 |
| genome | |
| Pelamoviroid MK188695.1 Apple hammerhead viroid isolate SD17_9-2, complete | SEQ ID NO: 56 |
| genome | |
| Pelamoviroid MK188693.1 Apple hammerhead viroid isolate SD17_4-1, complete | SEQ ID NO: 57 |
| genome | |
| Pelamoviroid MH643727.1 Apple hammerhead viroid strain AHVd-IT_MRG_16, | SEQ ID NO: 58 |
| complete sequence | |
| Pelamoviroid MH643711.1 Apple hammerhead viroid strain AHVd-IT_Ag_16, | SEQ ID NO: 59 |
| complete sequence | |
| Pelamoviroid MH643700.1 Apple hammerhead viroid strain AHVd-IT_Ag_05, | SEQ ID NO: 60 |
| complete sequence | |
| Pelamoviroid MK188705.1 Apple hammerhead viroid isolate AP_SD15_APP1_1, | SEQ ID NO: 61 |
| complete genome | |
| Pelamoviroid MK188703.1 Apple hammerhead viroid isolate SD18_16-1, | SEQ ID NO: 62 |
| complete genome | |
| Pelamoviroid MK188702.1 Apple hammerhead viroid isolate SD18_6-5, complete | SEQ ID NO: 63 |
| genome | |
| Pelamoviroid MK188692.1 Apple hammerhead viroid isolate SD17_3-3, complete | SEQ ID NO: 64 |
| genome | |
| Pospiviroid AY671955.1 Citrus exocortis viroid isolate D-43, complete genome | SEQ ID NO: 65 |
| Pospiviroid AY671956.1 Citrus exocortis viroid isolate D-40, complete genome | SEQ ID NO: 66 |
| Pelamoviroid MH049334.1 Apple hammerhead viroid isolate | SEQ ID NO: 67 |
| AHVd_17_New_Zealand, partial genome | |
| Pospiviroid LQ907271.1 Sequence 53 from Patent WO2018179834 | SEQ ID NO: 68 |
| Pelamoviroid AJ247113.1 Chrysanthemum chlorotic mottle viroid RNA, variant | SEQ ID NO: 69 |
| CMNS 2 | |
| Pelamoviroid KT033024.1 Chrysanthemum chlorotic mottle viroid isolate Ford-19, | SEQ ID NO: 70 |
| complete genome | |
| Pelamoviroid DQ402041.1 Chrysanthemum chlorotic mottle viroid RNA, complete | SEQ ID NO: 71 |
| genome | |
| Pelamoviroid LC089753.1 Chrysanthemum chlorotic mottle viroid genomic RNA, | SEQ ID NO: 72 |
| complete genome, strain: K6pop | |
| Pelamoviroid AJ878089.1 Chrysanthemum chlorotic mottle viroid, clone CM298 | SEQ ID NO: 73 |
| VR | |
| Apscaviroid NC_010308.1 Persimmon viroid, complete genome | SEQ ID NO: 74 |
| Pospiviroid FJ751933.1 Citrus exocortis Yucatan viroid isolate 15, complete | SEQ ID NO: 75 |
| genome | |
| Apscaviroid KP010010.1 Grapevine yellow speckle viroid 1 clone Poochidpha-3, | SEQ ID NO: 76 |
| complete genome | |
| Pospiviroid KC290928.1 Citrus exocortis viroid strain CMC-D, complete genome | SEQ ID NO: 77 |
| Pospiviroid JQ974378.1 Tomato chlorotic dwarf viroid clone 1, complete genome | SEQ ID NO: 78 |
| Pospiviroid MH771135.1 Citrus exocortis viroid isolate CEVd-CY218, complete | SEQ ID NO: 79 |
| genome | |
| Apscaviroid MG780426.1 Grapevine yellow speckle viroid 2 clone 2, complete | SEQ ID NO: 80 |
| genome | |
| Apscaviroid KY404214.1 Australian grapevine viroid isolate AGVd-Iran6, complete | SEQ ID NO: 81 |
| genome | |
| Apscaviroid HM211854.1 Australian grapevine viroid isolate F3, complete genome | SEQ ID NO: 82 |
| Apscaviroid AB429168.1 Apple fruit crinkle viroid RNA, Apscaviroid central | SEQ ID NO: 83 |
| conserved region, strain: hop isolate, clone: HY-A25 | |
| Pospiviroid AY372393.1 Citrus exocortis viroid isolate 89002600, complete | SEQ ID NO: 84 |
| genome | |
| Pospiviroid KY810771.1 Columnea latent viroid strain FERA_160205, complete | SEQ ID NO: 85 |
| genome | |
| Pospiviroid HG739076.1 Citrus exocortis viroid, complete sequence, isolate | SEQ ID NO: 86 |
| 11/0171/VI.7 | |
| Pospiviroid M30870.1 Citrus exocortis viroid (CEV-JB) complete genome | SEQ ID NO: 87 |
| Apscaviroid LC199972.1 Apple fruit crinkle viroid genomic RNA, complete | SEQ ID NO: 88 |
| genome, clone: HS1-T | |
| Pospiviroid HM043818.1 Columnea latent viroid isolate 14 clone 2, complete | SEQ ID NO: 89 |
| genome | |
| Pospiviroid AY367350.1 Columnea latent viroid isolate 89001013, complete | SEQ ID NO: 90 |
| genome | |
| Pospiviroid X95292.1 Columnea latent viroid-B stem-loop RNA | SEQ ID NO: 91 |
| Pospiviroid KY110721.1 Citrus exocortis viroid strain R140902-18, complete | SEQ ID NO: 92 |
| genome | |
| Pospiviroid KY473932.1 Citrus exocortis viroid isolate Ex-SR-Ver, complete | SEQ ID NO: 93 |
| genome | |
| Pospiviroid S67441.1 viroid genome [citrus exocortis viroid CEV, isolate tomato | SEQ ID NO: 94 |
| hybrid, CEVt, Genomic Complete, 372 nt] | |
| Pospiviroid DQ431992.1 Citrus exocortis viroid isolate CEVd-XNM-HJ | SEQ ID NO: 95 |
| Guangdong-YC, complete genome | |
| Pospiviroid X53716.1 L. esculentum citrus exocortis viorid-tomato (CEVd-t) RNA | SEQ ID NO: 96 |
| Apscaviroid AB429216.1 Apple fruit crinkle viroid RNA, Apscaviroid central | SEQ ID NO: 97 |
| conserved region, strain: apple isolate, clone: AA-J14 | |
| Pospiviroid KR020040.1 Iresine viroid 1 isolate Cp4, complete genome | SEQ ID NO: 98 |
| Pospiviroid JF446930.1 Columnea latent viroid isolate CM10-6c2, complete | SEQ ID NO: 99 |
| genome | |
| Pospiviroid EF126047.1 Citrus exocortis viroid isolate LMPE7, complete genome | SEQ ID NO: 100 |
| Pospiviroid EF015581.1 Columnea latent viroid, complete genome | SEQ ID NO: 101 |
| Pospiviroid DQ431994.1 Citrus exocortis viroid isolate CEVd-Italy-BL China, | SEQ ID NO: 102 |
| complete genome | |
| Pospiviroid JF742633.1 Columnea latent viroid clone Solanum 4, complete | SEQ ID NO: 103 |
| genome | |
| Pospiviroid KY646193.1 Citrus exocortis viroid isolate PTZ57R/T, complete | SEQ ID NO: 104 |
| genome | |
| Pospiviroid KY473931.1 Citrus exocortis viroid isolate Ex-CA-Ver, complete | SEQ ID NO: 105 |
| genome | |
| Pospiviroid DQ471994.1 Citrus exocortis viroid isolate CSC09, complete genome | SEQ ID NO: 106 |
| Pospiviroid Y00328.1 Citrus exocortis viroid (CEV-g) RNA genome | SEQ ID NO: 107 |
| Apscaviroid MG010378.1 Grapevine yellow speckle viroid 1 isolate Volovnik AV5, | SEQ ID NO: 108 |
| complete genome | |
| Apscaviroid MF510389.1 Grapevine yellow speckle viroid 1 isolate GYSV1-HUHT, | SEQ ID NO: 109 |
| complete genome | |
| Apscaviroid LC500206.1 Japanese grapevine viroid Na RNA, complete genome | SEQ ID NO: 110 |
| Apscaviroid MH476216.1 Grapevine yellow speckle viroid 1, complete genome | SEQ ID NO: 111 |
| Apscaviroid KX966270.1 Grapevine yellow speckle viroid 1 clone 9s CZZ, | SEQ ID NO: 112 |
| complete genome | |
| Apscaviroid KT000352.1 Grapevine yellow speckle viroid 1 isolate 22_2_3, | SEQ ID NO: 113 |
| complete genome | |
| Apscaviroid LT601587.1 Grapevine yellow speckle viroid 2, complete sequence, | SEQ ID NO: 114 |
| isolate KO | |
| Apscaviroid KU880715.1 Grapevine yellow speckle viroid 1 isolate SY-BR, | SEQ ID NO: 115 |
| complete sequence | |
| Apscaviroid KF007320.1 Grapevine yellow speckle viroid 2 isolate | SEQ ID NO: 116 |
| 6703_YS2_ThS2, complete genome | |
| Apscaviroid DQ371477.1 Grapevine yellow speckle viroid 1 clone Tu CGVd 1-17, | SEQ ID NO: 117 |
| complete genome | |
| Apscaviroid AB028465.1 Grapevine yellow speckle viroid 1 mRNA, the sequence | SEQ ID NO: 118 |
| of yellow speckle symptom | |
| Apscaviroid MK804769.1 Grapevine yellow speckle viroid 1 isolate GYSV1.NUB- | SEQ ID NO: 119 |
| BR, complete genome | |
| Apscaviroid KU880716.1 Grapevine yellow speckle viroid 1 isolate TR-BR, | SEQ ID NO: 120 |
| complete sequence | |
| Apscaviroid KU880714.1 Grapevine yellow speckle viroid 1 isolate MH-BR, | SEQ ID NO: 121 |
| complete sequence | |
| Apscaviroid KU880713.1 Grapevine yellow speckle viroid 1 isolate RM-BR, | SEQ ID NO: 122 |
| complete sequence | |
| Pospiviroid AY372390.1 Citrus exocortis viroid isolate 8900808, complete genome | SEQ ID NO: 123 |
| Pospiviroid HQ667139.1 Tomato apical stunt viroid isolate sj3, complete genome | SEQ ID NO: 124 |
| Coleviroid NC_003683.1 Coleus blumei viroid 3, complete genome | SEQ ID NO: 125 |
| Pospiviroid MG132058.1 Tomato apical stunt viroid isolate Touboudan, complete | SEQ ID NO: 126 |
| sequence | |
| Pospiviroid MG132057.1 Tomato apical stunt viroid isolate Akumadan, complete | SEQ ID NO: 127 |
| sequence | |
| Pospiviroid KX579067.1 Tomato apical stunt viroid isolate 5458774, complete | SEQ ID NO: 128 |
| genome | |
| Pospiviroid KJ956800.1 Citrus exocortis viroid isolate 2 from lemon, complete | SEQ ID NO: 129 |
| genome | |
| Pospiviroid KF437621.1 Tomato apical stunt viroid clone TASVd_31, complete | SEQ ID NO: 130 |
| genome | |
| Pospiviroid AY372394.1 Potato spindle tuber viroid isolate 94005977, complete | SEQ ID NO: 131 |
| genome | |
| Pospiviroid MK286920.1 Tomato apical stunt viroid isolate Al-Kharj-SA2, complete | SEQ ID NO: 132 |
| genome | |
| Coleviroid MK477627.1 Coleus blumei viroid 3 isolate CbVd3_V_10_DOM, | SEQ ID NO: 133 |
| complete sequence | |
| Pospiviroid KY522737.1 Potato spindle tuber viroid isolate MT41ozon, complete | SEQ ID NO: 134 |
| genome | |
| Hostuviroid AF462154.1 Grapevine viroid clone Syrah complete genome | SEQ ID NO: 135 |
| Pospiviroid KP863711.1 Mexican papita viroid isolate chimera 3, complete | SEQ ID NO: 136 |
| sequence | |
| Pospiviroid GQ131572.1 Tomato chlorotic dwarf viroid isolate Mex9580, complete | SEQ ID NO: 137 |
| genome | |
| Pospiviroid NC_001558.1 Tomato planta macho viroid, complete genome | SEQ ID NO: 138 |
| Pospiviroid JQ975098.1 Tomato chlorotic dwarf viroid, complete genome | SEQ ID NO: 139 |
| Pospiviroid X95293.1 Tomato apical stunt viroid-S stem-loop RNA | SEQ ID NO: 140 |
| Pospiviroid HQ452399.1 Potato spindle tuber viroid isolate ca1a, complete | SEQ ID NO: 141 |
| genome | |
| Pospiviroid FJ797614.1 Potato spindle tuber viroid isolate 092009595, complete | SEQ ID NO: 142 |
| genome | |
| Pospiviroid AY222078.1 Columnea latent viroid isolate PSTVd*/CLVd, complete | SEQ ID NO: 143 |
| genome | |
| Apscaviroid NC_021720.1 Persimmon viroid 2 genomic RNA, complete genome | SEQ ID NO: 144 |
| Pospiviroid MK330995.1 Potato spindle tuber viroid isolate 21809902-95 | SEQ ID NO: 145 |
| sequence | |
| Pospiviroid KJ857498.1 Potato spindle tuber viroid isolate CVN212, complete | SEQ ID NO: 146 |
| genome | |
| Pospiviroid NC_002015.1 Chrysanthemum stunt viroid, complete genome | SEQ ID NO: 147 |
| Pospiviroid LC192496.1 Chrysanthemum stunt viroid genomic RNA, complete | SEQ ID NO: 148 |
| genome, isolate: US2.111 | |
| Pospiviroid LC192474.1 Chrysanthemum stunt viroid genomic RNA, complete | SEQ ID NO: 149 |
| genome, isolate: US2.51 | |
| Pospiviroid KX096422.1 Chrysanthemum stunt viroid isolate Ford-26, complete | SEQ ID NO: 150 |
| sequence | |
| Apscaviroid MF521431.2 Apple chlorotic fruit spot viroid, complete genome | SEQ ID NO: 151 |
| Pelamoviroid JN377886.1 Peach latent mosaic viroid clone C40-A349-g12.83, | SEQ ID NO: 152 |
| complete sequence | |
| Pospiviroid KP262537.1 Chrysanthemum stunt viroid isolate CSVd-4, complete | SEQ ID NO: 153 |
| genome | |
| Pospiviroid KP262535.1 Chrysanthemum stunt viroid isolate CSVd-2, complete | SEQ ID NO: 154 |
| genome | |
| Apscaviroid KY473930.1 Citrus dwarfing viroid isolate En-CT-Ver, complete | SEQ ID NO: 155 |
| genome | |
| Pelamoviroid DQ680797.1 Peach latent mosaic viroid variant 94.2, complete | SEQ ID NO: 156 |
| genome | |
| Pelamoviroid DQ222090.1 Peach latent mosaic viroid isolate PC-P1.148.73, | SEQ ID NO: 157 |
| complete genome | |
| Pelamoviroid AJ550910.1 Peach latent mosaic viroid complete genome, isolate | SEQ ID NO: 158 |
| PC-C37 | |
| Pospiviroid NC_027432.1 Portulaca latent viroid isolate Vd21, complete genome | SEQ ID NO: 159 |
| Pelamoviroid JN377883.1 Peach latent mosaic viroid clone C40-stem-g12, | SEQ ID NO: 160 |
| complete sequence | |
| Pelamoviroid GQ872137.1 Peach latent mosaic viroid isolate Laura.4, complete | SEQ ID NO: 161 |
| genome | |
| Pelamoviroid DQ680799.1 Peach latent mosaic viroid variant 88.1, complete | SEQ ID NO: 162 |
| genome | |
| Pelamoviroid DQ222055.1 Peach latent mosaic viroid PC-P2.33, complete | SEQ ID NO: 163 |
| genome | |
| Pelamoviroid DQ222053.1 Peach latent mosaic viroid PC-P1.151, complete | SEQ ID NO: 164 |
| genome | |
| Pelamoviroid DQ222074.1 Peach latent mosaic viroid isolate PC-P1.142.12, | SEQ ID NO: 165 |
| complete genome | |
| Pospiviroid JF446895.1 Pepper chat fruit viroid isolate NK10-6c4, complete | SEQ ID NO: 166 |
| genome | |
| Pelamoviroid GQ872138.1 Peach latent mosaic viroid isolate Luara.5, complete | SEQ ID NO: 167 |
| genome | |
| Pospiviroid JF446909.1 Pepper chat fruit viroid isolate LP2-5c4, complete genome | SEQ ID NO: 168 |
| Coleviroid FJ615419.1 Coleus blumei viroid 6 clone 2, complete genome | SEQ ID NO: 169 |
| Hostuviroid MK496633.1 Dahlia latent viroid isolate DLV/CU, complete sequence | SEQ ID NO: 170 |
| Pelamoviroid KJ754183.1 Peach latent mosaic viroid isolate SDP2, complete | SEQ ID NO: 171 |
| sequence | |
| Pelamoviroid KF870319.1 Peach latent mosaic viroid clone 1893_5_341 clone-lib | SEQ ID NO: 172 |
| P3, complete sequence | |
| Pelamoviroid DQ680798.1 Peach latent mosaic viroid variant 235.2, complete | SEQ ID NO: 173 |
| genome | |
| Pelamoviroid DQ680718.1 Peach latent mosaic viroid variant 278.3, complete | SEQ ID NO: 174 |
| genome | |
| Apscaviroid FJ974062.1 Apple scar skin viroid clone Lynkestis-1, complete | SEQ ID NO: 175 |
| genome | |
| Pelamoviroid MG788242.1 Peach latent mosaic viroid clone D4, complete | SEQ ID NO: 176 |
| genome | |
| Pelamoviroid MF574160.1 Peach latent mosaic viroid isolate MH35, complete | SEQ ID NO: 177 |
| genome | |
| Pelamoviroid MF574156.1 Peach latent mosaic viroid isolate BH5, complete | SEQ ID NO: 178 |
| genome | |
| Pelamoviroid KY355321.1 Peach latent mosaic viroid isolate PLMVd-JT-P1-24, | SEQ ID NO: 179 |
| complete genome | |
| Pelamoviroid KY355281.1 Peach latent mosaic viroid isolate PLMVd-JH-P1-25, | SEQ ID NO: 180 |
| complete genome | |
| Pelamoviroid DQ680801.1 Peach latent mosaic viroid variant 235.4, complete | SEQ ID NO: 181 |
| genome | |
| Pelamoviroid DQ680779.1 Peach latent mosaic viroid variant 133′.1, complete | SEQ ID NO: 182 |
| genome | |
| Pelamoviroid KY355220.1 Peach latent mosaic viroid isolate PLMVd-CH-P1-19, | SEQ ID NO: 183 |
| complete genome | |
| Pelamoviroid KY355176.1 Peach latent mosaic viroid isolate PLMVd-BC-P1-19, | SEQ ID NO: 184 |
| complete genome | |
| Pelamoviroid KY310588.1 Peach latent mosaic viroid isolate JH, complete | SEQ ID NO: 185 |
| genome | |
| Pelamoviroid KU048793.1 Peach latent mosaic viroid isolate plum1.3, complete | SEQ ID NO: 186 |
| genome | |
| Pelamoviroid KF870034.1 Peach latent mosaic viroid clone 1593_5_339 clone-lib | SEQ ID NO: 187 |
| P3, complete sequence | |
| Pelamoviroid JX480364.1 Peach latent mosaic viroid clone 966_9_339 clone-lib | SEQ ID NO: 188 |
| P3, complete sequence | |
| Pelamoviroid GU825983.1 Peach latent mosaic viroid clone PLMVd-2tun, | SEQ ID NO: 189 |
| complete genome | |
| Pelamoviroid DQ839564.1 Peach latent mosaic viroid clone Eg18, complete | SEQ ID NO: 190 |
| genome | |
| Pelamoviroid DQ680806.1 Peach latent mosaic viroid variant 387.1, complete | SEQ ID NO: 191 |
| genome | |
| Pelamoviroid DQ680789.1 Peach latent mosaic viroid variant 330.1, complete | SEQ ID NO: 192 |
| genome | |
| Pelamoviroid DQ680783.1 Peach latent mosaic viroid variant 369.4, complete | SEQ ID NO: 193 |
| genome | |
| Pelamoviroid DQ680774.1 Peach latent mosaic viroid variant 369.1, complete | SEQ ID NO: 194 |
| genome | |
| Pelamoviroid AF170504.1 Peach latent mosaic viroid variant #11 from Prunus | SEQ ID NO: 195 |
| persica Redhaven cultivar, complete sequence | |
| Pospiviroid MH995516.1 Citrus exocortis viroid isolate VC VIV 3 M20, partial | SEQ ID NO: 196 |
| genome | |
| Pelamoviroid MH974830.1 Peach latent mosaic viroid isolate T02-3, complete | SEQ ID NO: 197 |
| genome | |
| Pelamoviroid MK212087.1 Peach latent mosaic viroid isolate y7-pgl6, complete | SEQ ID NO: 198 |
| genome | |
| Pelamoviroid MK212050.1 Peach latent mosaic viroid isolate y2-p5, complete | SEQ ID NO: 199 |
| genome | |
| Pelamoviroid KY355380.1 Peach latent mosaic viroid isolate PLMVd-MB-P2-19, | SEQ ID NO: 200 |
| complete genome | |
| Pelamoviroid KY355322.1 Peach latent mosaic viroid isolate PLMVd-JT-P2-1, | SEQ ID NO: 201 |
| complete genome | |
| Pelamoviroid KY310586.1 Peach latent mosaic viroid isolate BC, complete | SEQ ID NO: 202 |
| genome | |
| Pelamoviroid KF417551.1 Peach latent mosaic viroid isolate 96, complete | SEQ ID NO: 203 |
| genome | |
| Pelamoviroid JX479733.1 Peach latent mosaic viroid clone 335_33_338 clone-lib | SEQ ID NO: 204 |
| P3, complete sequence | |
| Pelamoviroid HM185115.1 Peach latent mosaic viroid isolate AY09AT5 clonH2, | SEQ ID NO: 205 |
| complete genome | |
| Pelamoviroid GQ499321.1 Peach latent mosaic viroid isolate 17, complete | SEQ ID NO: 206 |
| sequence | |
| Pelamoviroid DQ680760.1 Peach latent mosaic viroid variant 111.2, complete | SEQ ID NO: 207 |
| genome | |
| Pelamoviroid AF339740.1 Peach latent mosaic viroid wild pear isolate, complete | SEQ ID NO: 208 |
| genome | |
| Pelamoviroid EU708837.1 Peach latent mosaic viroid clone 215.1, complete | SEQ ID NO: 209 |
| genome | |
| Pelamoviroid LC469622.1 Peach latent mosaic viroid RNA, complete genome | SEQ ID NO: 210 |
| Pelamoviroid KY355341.1 Peach latent mosaic viroid isolate PLMVd-JT-P2-23, | SEQ ID NO: 211 |
| complete genome | |
| Pelamoviroid KY355237.1 Peach latent mosaic viroid isolate PLMVd-CH-P2-20, | SEQ ID NO: 212 |
| complete genome | |
| Pelamoviroid KY355189.1 Peach latent mosaic viroid isolate PLMVd-BC-P2-10, | SEQ ID NO: 213 |
| complete genome | |
| Pelamoviroid KY310587.1 Peach latent mosaic viroid isolate CH, complete | SEQ ID NO: 214 |
| genome | |
| Pelamoviroid KX430168.1 Peach latent mosaic viroid isolate v2.7, complete | SEQ ID NO: 215 |
| sequence | |
| Pelamoviroid EU888599.1 Peach latent mosaic viroid isolate PchMx · Azt2, | SEQ ID NO: 216 |
| complete genome | |
| Pelamoviroid KF869589.1 Peach latent mosaic viroid clone 1128_8_337 clone-lib | SEQ ID NO: 217 |
| P3, complete sequence | |
| Pelamoviroid JX480348.1 Peach latent mosaic viroid clone 950_9_337 clone-lib | SEQ ID NO: 218 |
| P3, complete sequence | |
| Pelamoviroid JX479333.1 Peach latent mosaic viroid clone 1064_9_337 clone-lib | SEQ ID NO: 219 |
| P7, complete sequence | |
| Pelamoviroid AJ550900.1 Peach latent mosaic viroid complete genome, isolate | SEQ ID NO: 220 |
| PC-C34 | |
| Pelamoviroid AF170523.1 Peach latent mosaic viroid variant #4 from Prunus | SEQ ID NO: 221 |
| persica Tylor cultivar, complete sequence | |
| Pelamoviroid MH974831.1 Peach latent mosaic viroid isolate T02-6, complete | SEQ ID NO: 222 |
| genome | |
| Pelamoviroid KY355241.1 Peach latent mosaic viroid isolate PLMVd-CH-P2-24, | SEQ ID NO: 223 |
| complete genome | |
| Pelamoviroid JX479340.2 Peach latent mosaic viroid clone 1071_9_336 clone-lib | SEQ ID NO: 224 |
| P7, complete sequence | |
| Pelamoviroid AF339739.1 Peach latent mosaic viroid peach isolate, complete | SEQ ID NO: 225 |
| genome | |
| Apscaviroid FJ974092.1 Apple scar skin viroid clone Leuki 30.1, complete | SEQ ID NO: 226 |
| genome | |
| Elaviroid KT901908.1 Eggplant latent viroid clone ELVd2-74, complete genome | SEQ ID NO: 227 |
| Elaviroid KT901890.1 Eggplant latent viroid clone ELVd2-56, complete genome | SEQ ID NO: 228 |
| Elaviroid KT901868.1 Eggplant latent viroid clone ELVd2-34, complete genome | SEQ ID NO: 229 |
| Elaviroid AJ536620.1 Eggplant latent viroid complete genome, isolate 9 | SEQ ID NO: 230 |
| Elaviroid AJ536617.1 Eggplant latent viroid complete genome, isolate 6 | SEQ ID NO: 231 |
| Pelamoviroid AJ241843.1 peach latent mosaic viroid, varient esc10-6b | SEQ ID NO: 232 |
| Apscaviroid MN598209.1 Apple scar skin viroid isolate ASSVd PQ-n, complete | SEQ ID NO: 233 |
| sequence | |
| Apscaviroid MK102984.1 Apple scar skin viroid isolate QDHT-4, complete | SEQ ID NO: 234 |
| genome | |
| Apscaviroid HQ326087.1 Apple scar skin viroid isolate AY106, complete genome | SEQ ID NO: 235 |
| Apscaviroid EU144226.1 Citrus bent leaf viroid isolate Anliucheng-1, complete | SEQ ID NO: 236 |
| genome | |
| Elaviroid KT901928.1 Eggplant latent viroid clone ELVd2-94, complete genome | SEQ ID NO: 237 |
| Elaviroid KT901909.1 Eggplant latent viroid clone ELVd2-75, complete genome | SEQ ID NO: 238 |
| Elaviroid KT901902.1 Eggplant latent viroid clone ELVd2-68, complete genome | SEQ ID NO: 239 |
| Elaviroid KT901858.1 Eggplant latent viroid clone ELVd2-24, complete genome | SEQ ID NO: 240 |
| Elaviroid AJ536615.1 Eggplant latent viroid complete genome, isolate 4 | SEQ ID NO: 241 |
| Pospiviroid MF770198.1 Columnea latent viroid isolate TS03, partial genome | SEQ ID NO: 242 |
| Apscaviroid MN598207.1 Apple scar skin viroid isolate ASSVd PQ-hg, complete | SEQ ID NO: 243 |
| sequence | |
| Apscaviroid KC769198.1 Citrus viroid VI isolate SE-1, complete sequence | SEQ ID NO: 244 |
| Elaviroid KT901918.1 Eggplant latent viroid clone ELVd2-84, complete genome | SEQ ID NO: 245 |
| Elaviroid KT901916.1 Eggplant latent viroid clone ELVd2-82, complete genome | SEQ ID NO: 246 |
| Elaviroid KT901903.1 Eggplant latent viroid clone ELVd2-69, complete genome | SEQ ID NO: 247 |
| Apscaviroid KP765435.1 Apple scar skin viroid isolate Yeongwol, complete | SEQ ID NO: 248 |
| genome | |
| Apscaviroid EU031486.1 Apple scar skin viroid variant ASSVd_PE10, complete | SEQ ID NO: 249 |
| genome | |
| Apscaviroid MN598214.1 Apple scar skin viroid isolate ASSVd LY-qb, complete | SEQ ID NO: 250 |
| sequence | |
| Apscaviroid MH476217.1 Australian grapevine viroid, complete genome | SEQ ID NO: 251 |
| Apscaviroid LC431516.1 Apple scar skin viroid GH1 RNA, complete genome | SEQ ID NO: 252 |
| Apscaviroid LN823956.1 Apple scar skin viroid, complete sequence, isolate | SEQ ID NO: 253 |
| DHWH1 | |
| Elaviroid KT901906.1 Eggplant latent viroid clone ELVd2-72, complete genome | SEQ ID NO: 254 |
| Apscaviroid LN823957.1 Apple scar skin viroid, complete sequence, isolate | SEQ ID NO: 255 |
| DHWH2 | |
| Apscaviroid EU031496.1 Apple scar skin viroid variant ASSVd_AP10, complete | SEQ ID NO: 256 |
| genome | |
| Apscaviroid EU031466.1 Apple scar skin viroid variant ASSVd_AM12, complete | SEQ ID NO: 257 |
| genome | |
| Apscaviroid AB054640.1 Citrus viroid-I-LSS variant WS2 genomic RNA, complete | SEQ ID NO: 258 |
| sequence | |
| Apscaviroid MH013320.1 Apple scar skin viroid isolate JZ1-4, complete genome | SEQ ID NO: 259 |
| Apscaviroid KY114496.1 Australian grapevine viroid isolate TR188-Antep Karasi, | SEQ ID NO: 260 |
| partial genome | |
| Apscaviroid KY114494.1 Australian grapevine viroid isolate TR179-Antep Karasi, | SEQ ID NO: 261 |
| partial genome | |
| Apscaviroid KY114492.1 Australian grapevine viroid isolate TR189-Ceviz Uzumu, | SEQ ID NO: 262 |
| partial genome | |
| Apscaviroid KP772685.1 Apple scar skin viroid isolate S-q6-10, complete genome | SEQ ID NO: 263 |
| Apscaviroid KF726092.1 Citrus viroid-I-LSS isolate SL-4, complete genome | SEQ ID NO: 264 |
| Apscaviroid HG764206.1 Apple scar skin viroid, complete sequence, isolate Q1 | SEQ ID NO: 265 |
| Apscaviroid GQ249350.1 Apple scar skin viroid isolate Vassiliki-680, complete | SEQ ID NO: 266 |
| genome | |
| Apscaviroid M36646.1 Apple scar skin viroid, complete sequence | SEQ ID NO: 267 |
| Pospiviroid MF770202.1 Columnea latent viroid isolate TS08, partial genome | SEQ ID NO: 268 |
| Apscaviroid KP772687.1 Apple scar skin viroid isolate S-T37-11, complete | SEQ ID NO: 269 |
| genome | |
| Apscaviroid MH200820.1 Citrus bent leaf viroid isolate CBLVd-GH-CI13, complete | SEQ ID NO: 270 |
| genome | |
| Apscaviroid JX861259.1 Apple scar skin viroid clone ASSVd-Z7, complete | SEQ ID NO: 271 |
| genome | |
| Pelamoviroid KF870227.1 Peach latent mosaic viroid clone 1799_5_323 clone-lib | SEQ ID NO: 272 |
| P3, complete sequence | |
| Pelamoviroid KF867087.1 Peach latent mosaic viroid clone 703_16_323 clone-lib | SEQ ID NO: 273 |
| P7, complete sequence | |
| Pospiviroid MF770197.1 Pepper chat fruit viroid isolate TGS17s, partial genome | SEQ ID NO: 274 |
| Pelamoviroid MK212064.1 Peach latent mosaic viroid isolate y4-pg4, complete | SEQ ID NO: 275 |
| genome | |
| Pospiviroid EU273604.1 Potato spindle tuber viroid, partial sequence | SEQ ID NO: 276 |
| Apscaviroid KY654680.1 Citrus bent leaf viroid isolate PTZ 57R/T, complete | SEQ ID NO: 277 |
| genome | |
| Cocadviroid MK795526.1 Hop latent viroid strain CV_7, complete genome | SEQ ID NO: 278 |
| Apscaviroid MN734784.1 Plum viroid I isolate ZR6, complete genome | SEQ ID NO: 279 |
| Apscaviroid M74065.1 Citrus bent leaf viroid RNA | SEQ ID NO: 280 |
| Apscaviroid HQ606079.1 Pear blister canker viroid, complete sequence | SEQ ID NO: 281 |
| Apscaviroid GU825972.1 Pear blister canker viroid clone PBCV5, complete | SEQ ID NO: 282 |
| genome | |
| Apscaviroid GQ141740.1 Pear blister canker viroid isolate Tzotzis, complete | SEQ ID NO: 283 |
| genome | |
| Apscaviroid DQ186640.1 Pear blister canker viroid PBCVd isolate 35, complete | SEQ ID NO: 284 |
| genome | |
| Apscaviroid FJ974082.1 Pear blister canker viroid isolate Leuki-30, complete | SEQ ID NO: 285 |
| genome | |
| Hostuviroid KY508372.1 Hop stunt viroid isolate Xi-AR-Ver, complete genome | SEQ ID NO: 286 |
| Apscaviroid FJ974088.1 Pear blister canker viroid clone Velistiche, complete | SEQ ID NO: 287 |
| genome | |
| Apscaviroid EU978464.1 Pear blister canker viroid isolate PEK, complete genome | SEQ ID NO: 288 |
| Pospiviroid FJ031232.1 Chrysanthemum stunt viroid isolate Beijing, partial | SEQ ID NO: 289 |
| sequence | |
| Hostuviroid KY508370.1 Hop stunt viroid isolate Xi-MT-Ver, complete genome | SEQ ID NO: 290 |
| Hostuviroid KY508369.1 Hop stunt viroid isolate Xi-CA-Ver, complete genome | SEQ ID NO: 291 |
| Apscaviroid KF788291.1 Apple dimple fruit viroid, complete sequence | SEQ ID NO: 292 |
| Hostuviroid KJ810551.1 Hop stunt viroid clone MO-D5m, complete genome | SEQ ID NO: 293 |
| Hostuviroid LC507888.1 Hop stunt viroid HSVd-US RNA, complete genome | SEQ ID NO: 294 |
| Apscaviroid MG516817.1 Apple dimple fruit viroid isolate IRN9, complete genome | SEQ ID NO: 295 |
| Apscaviroid MG516811.1 Apple dimple fruit viroid isolate IRN3, complete genome | SEQ ID NO: 296 |
| Apscaviroid KX909575.1 Apple dimple fruit viroid isolate IRN1, complete | SEQ ID NO: 297 |
| sequence | |
| Hostuviroid MH476218.1 Hop stunt viroid, complete genome | SEQ ID NO: 298 |
| Hostuviroid JQ080274.1 Hop stunt viroid isolate HSVdr, complete genome | SEQ ID NO: 299 |
| Apscaviroid NC_035620.1 Lychee viroid-like RNA isolate 20160105, complete | SEQ ID NO: 300 |
| sequence | |
| Apscaviroid LC214530.1 Apple dimple fruit viroid genomic RNA, complete | SEQ ID NO: 301 |
| genome, isolate: ADFVd-J, clone: JG16-1 | |
| Hostuviroid MH537090.1 Hop stunt viroid isolate SC-20, complete genome | SEQ ID NO: 302 |
| Hostuviroid MF576418.1 Hop stunt viroid isolate DgHV-3, complete genome | SEQ ID NO: 303 |
| Hostuviroid KT000360.1 Hop stunt viroid isolate 22_2_3, complete genome | SEQ ID NO: 304 |
| Hostuviroid KU640955.1 Hop stunt viroid isolate HSVd-Mor-ValL_Big, complete | SEQ ID NO: 305 |
| sequence | |
| Hostuviroid KP126952.1 Hop stunt viroid clone HSVd-F1, complete genome | SEQ ID NO: 306 |
| Hostuviroid FJ626867.1 Hop stunt viroid isolate e, complete genome | SEQ ID NO: 307 |
| Coleviroid NC_003682.1 Coleus blumei viroid 2, complete genome | SEQ ID NO: 308 |
| Hostuviroid AY513267.1 Citrus cachexia viroid, complete genome | SEQ ID NO: 309 |
| Hostuviroid KM213398.1 Hop stunt viroid isolate HR174339, complete sequence | SEQ ID NO: 310 |
| Hostuviroid GQ260206.1 Hop stunt viroid isolate IIbI1, complete genome | SEQ ID NO: 311 |
| Apscaviroid EU382205.1 Citrus viroid III isolate CQ clone 1, complete genome | SEQ ID NO: 312 |
| Hostuviroid KP126944.1 Hop stunt viroid clone HSVd-F8, complete genome | SEQ ID NO: 313 |
| Apscaviroid KY110718.1 Citrus dwarfing viroid strain R140910-12, complete | SEQ ID NO: 314 |
| genome | |
| Apscaviroid GQ260210.1 Citrus dwarfing viroid isolate IIIaI2, complete genome | SEQ ID NO: 315 |
| Cocadviroid KT923143.1 Coconut cadang-cadang viroid, complete genome | SEQ ID NO: 316 |
| Hostuviroid KF007324.1 Hop stunt viroid isolate 6931_HSVd_Char, complete | SEQ ID NO: 317 |
| genome | |
| Hostuviroid KC677729.1 Hop stunt viroid isolate 332, complete sequence | SEQ ID NO: 318 |
| Hostuviroid FJ974079.1 Hop stunt viroid isolate Kernitsa-327, complete genome | SEQ ID NO: 319 |
| Hostuviroid DQ444476.1 Hop stunt viroid isolate HNiagD08, complete genome | SEQ ID NO: 320 |
| Apscaviroid LC500205.1 Hop stunt viroid CB RNA, complete genome | SEQ ID NO: 321 |
| Apscaviroid KT725632.1 Citrus dwarfing viroid isolate NRCV04, complete | SEQ ID NO: 322 |
| sequence | |
| Hostuviroid AF100641.1 Hop stunt viroid strain Kh, complete genome | SEQ ID NO: 323 |
| Coleviroid NC_003882.1 Coleus blumei viroid, complete genome | SEQ ID NO: 324 |
| Apscaviroid MN640603.1 Citrus viroid V isolate F19, complete genome | SEQ ID NO: 325 |
| Apscaviroid MF477874.1 Citrus viroid V isolate CVd-V-VC-8, complete genome | SEQ ID NO: 326 |
| Apscaviroid MF421247.1 Citrus dwarfing viroid clone CV18, complete genome | SEQ ID NO: 327 |
| Apscaviroid KC460712.1 Citrus viroid V isolate WN4R, complete genome | SEQ ID NO: 328 |
| Cocadviroid MK795572.1 Hop latent viroid strain CV_53, complete genome | SEQ ID NO: 329 |
| Apscaviroid KY110720.1 Citrus viroid V strain R141013-19, complete genome | SEQ ID NO: 330 |
| Apscaviroid KC460711.1 Citrus viroid V isolate TN1R, complete genome | SEQ ID NO: 331 |
| Apscaviroid JQ072089.1 Citrus viroid V from Citrus limon, complete genome | SEQ ID NO: 332 |
| Cocadviroid MK795541.1 Hop latent viroid strain CV_22, complete genome | SEQ ID NO: 333 |
| Cocadviroid MK795539.1 Hop latent viroid strain CV_20, complete genome | SEQ ID NO: 334 |
| Apscaviroid MF421250.1 Citrus dwarfing viroid clone CV32, complete genome | SEQ ID NO: 335 |
| Apscaviroid KY473929.1 Citrus dwarfing viroid isolate En-SR-Ver, complete | SEQ ID NO: 336 |
| genome | |
| Cocadviroid MK795563.1 Hop latent viroid strain CV_44, complete genome | SEQ ID NO: 337 |
| Cocadviroid MF421255.1 Citrus bark cracking viroid clone CC23, complete | SEQ ID NO: 338 |
| genome | |
| Cocadviroid MK795635.1 Hop latent viroid strain CV_116, complete genome | SEQ ID NO: 339 |
| Cocadviroid MK795520.1 Hop latent viroid strain CV_1, complete genome | SEQ ID NO: 340 |
| Cocadviroid MK795629.1 Hop latent viroid strain CV_110, complete genome | SEQ ID NO: 341 |
| Cocadviroid MK795551.1 Hop latent viroid strain CV_32, complete genome | SEQ ID NO: 342 |
| Cocadviroid MF198463.1 Citrus bark cracking viroid isolate W11, complete | SEQ ID NO: 343 |
| genome | |
| Cocadviroid MK795534.1 Hop latent viroid strain CV_15, complete genome | SEQ ID NO: 344 |
| Hostuviroid JX945670.1 Hop stunt viroid isolate 5018, partial sequence | SEQ ID NO: 345 |
| Cocadviroid MK795523.1 Hop latent viroid strain CV_4, complete genome | SEQ ID NO: 346 |
| Cocadviroid MG457782.1 Citrus bark cracking viroid isolate P2-1, complete | SEQ ID NO: 347 |
| genome | |
| Hostuviroid JX401927.1 Hop stunt viroid isolate 599, complete genome | SEQ ID NO: 348 |
| Cocadviroid MK795637.1 Hop latent viroid strain CV_118, complete genome | SEQ ID NO: 349 |
| Cocadviroid MK795631.1 Hop latent viroid strain CV_112, complete genome | SEQ ID NO: 350 |
| Cocadviroid MK795580.1 Hop latent viroid strain CV_61, complete genome | SEQ ID NO: 351 |
| Coleviroid MG767213.1 Coleus blumei viroid 5 clone 8, complete sequence | SEQ ID NO: 352 |
| Cocadviroid MK795586.1 Hop latent viroid strain CV_67, complete genome | SEQ ID NO: 353 |
| Apscaviroid MN379511.1 Citrus dwarfing viroid isolate Hawaii-3, partial genome | SEQ ID NO: 354 |
| Cocadviroid MK795553.1 Hop latent viroid strain CV_34, complete genome | SEQ ID NO: 355 |
| Cocadviroid MK795538.1 Hop latent viroid strain CV_19, complete genome | SEQ ID NO: 356 |
| Cocadviroid DQ097185.1 Coconut cadang-cadang viroid isolate oil palm variant | SEQ ID NO: 357 |
| 270, complete genome | |
| Cocadviroid MK795612.1 Hop latent viroid strain CV_93, complete genome | SEQ ID NO: 358 |
| Apscaviroid KY654684.1 Citrus viroid V isolate Ebi 1, partial genome | SEQ ID NO: 359 |
| Apscaviroid KY473927.1 Citrus dwarfing viroid isolate En-MT-Ver, complete | SEQ ID NO: 360 |
| genome | |
| Cocadviroid MK795596.1 Hop latent viroid strain CV_77, complete genome | SEQ ID NO: 361 |
| Cocadviroid MK795620.1 Hop latent viroid strain CV_101, complete genome | SEQ ID NO: 362 |
| Cocadviroid MK795537.1 Hop latent viroid strain CV_18, complete genome | SEQ ID NO: 363 |
| Cocadviroid MK795608.1 Hop latent viroid strain CV_89, complete genome | SEQ ID NO: 364 |
| Cocadviroid MF579864.1 Coconut cadang-cadang viroid isolate 323, complete | SEQ ID NO: 365 |
| genome | |
| Hostuviroid HE575348.1 Hop stunt viroid fragment, clone HSVd1, genomic RNA | SEQ ID NO: 366 |
| Cocadviroid MK795619.1 Hop latent viroid strain CV_100, complete genome | SEQ ID NO: 367 |
| Cocadviroid MK795617.1 Hop latent viroid strain CV_98, complete genome | SEQ ID NO: 368 |
| Cocadviroid MK795626.1 Hop latent viroid strain CV_107, complete genome | SEQ ID NO: 369 |
| Cocadviroid MK795615.1 Hop latent viroid strain CV_96, complete genome | SEQ ID NO: 370 |
| Cocadviroid MK795591.1 Hop latent viroid strain CV_72, complete genome | SEQ ID NO: 371 |
| Cocadviroid MK795548.1 Hop latent viroid strain CV_29, complete genome | SEQ ID NO: 372 |
| Cocadviroid MK795535.1 Hop latent viroid strain CV_16, complete genome | SEQ ID NO: 373 |
| Cocadviroid MK795581.1 Hop latent viroid strain CV_62, complete genome | SEQ ID NO: 374 |
| Cocadviroid MK795571.1 Hop latent viroid strain CV_52, complete genome | SEQ ID NO: 375 |
| Cocadviroid MK795561.1 Hop latent viroid strain CV_42, complete genome | SEQ ID NO: 376 |
| Cocadviroid MK795577.1 Hop latent viroid strain CV_58, complete genome | SEQ ID NO: 377 |
| Cocadviroid MK795527.1 Hop latent viroid strain CV_8, complete genome | SEQ ID NO: 378 |
| Cocadviroid KY654687.1 Citrus bark cracking viroid isolate Ebi 4, partial genome | SEQ ID NO: 379 |
| Cocadviroid HH970095.1 Sequence 7 from Patent EP1664298 | SEQ ID NO: 380 |
| Apscaviroid KY654686.1 Citrus dwarfing viroid isolate Ebi 3, partial genome | SEQ ID NO: 381 |
| Cocadviroid MK795583.1 Hop latent viroid strain CV_64, complete genome | SEQ ID NO: 382 |
| Cocadviroid KT600318.1 Hop latent viroid isolate GVdC_HLVd01, complete | SEQ ID NO: 383 |
| genome | |
| Cocadviroid MK795593.1 Hop latent viroid strain CV_74, complete genome | SEQ ID NO: 384 |
| Cocadviroid MK795589.1 Hop latent viroid strain CV_70, complete genome | SEQ ID NO: 385 |
| Cocadviroid NC_001471.1 Coconut tinangaja viroid, complete genome | SEQ ID NO: 386 |
| Cocadviroid MK795609.1 Hop latent viroid strain CV_90, complete genome | SEQ ID NO: 387 |
| Cocadviroid MK795605.1 Hop latent viroid strain CV_86, complete genome | SEQ ID NO: 388 |
| Cocadviroid MK795597.1 Hop latent viroid strain CV_78, complete genome | SEQ ID NO: 389 |
| Cocadviroid MK795576.1 Hop latent viroid strain CV_57, complete genome | SEQ ID NO: 390 |
| Avsunviroid EU888595.1 Avocado sunblotch viroid isolate AvdMx · Hass8, | SEQ ID NO: 391 |
| complete genome | |
| Cocadviroid MK795630.1 Hop latent viroid strain CV_111, complete genome | SEQ ID NO: 392 |
| Cocadviroid MK795628.1 Hop latent viroid strain CV_109, complete genome | SEQ ID NO: 393 |
| Cocadviroid MK795616.1 Hop latent viroid strain CV_97, complete genome | SEQ ID NO: 394 |
| Cocadviroid MK795549.1 Hop latent viroid strain CV_30, complete genome | SEQ ID NO: 395 |
| Cocadviroid MF579861.1 Coconut cadang-cadang viroid isolate 720, complete | SEQ ID NO: 396 |
| genome | |
| Coleviroid X95291.1 Coleus blumei viroid 1-RG stem-loop RNA | SEQ ID NO: 397 |
| Apscaviroid JX945672.1 Citrus dwarfing viroid isolate 6031, partial sequence | SEQ ID NO: 398 |
| Apscaviroid JX945669.1 Citrus bent leaf viroid isolate 4039, partial sequence | SEQ ID NO: 399 |
| Cocadviroid MK795633.1 Hop latent viroid strain CV_114, complete genome | SEQ ID NO: 400 |
| Coleviroid MK477626.1 Coleus blumei viroid 1 isolate CbVd1_6B_13, complete | SEQ ID NO: 401 |
| sequence | |
| Avsunviroid KF562707.1 Avocado sunblotch viroid isolate Uruapan-3, complete | SEQ ID NO: 402 |
| genome | |
| Cocadviroid MK795639.1 Hop latent viroid strain CV_120, complete genome | SEQ ID NO: 403 |
| Cocadviroid MK795614.1 Hop latent viroid strain CV_95, complete genome | SEQ ID NO: 404 |
| Cocadviroid MK795610.1 Hop latent viroid strain CV_91, complete genome | SEQ ID NO: 405 |
| Cocadviroid MK795567.1 Hop latent viroid strain CV_48, complete genome | SEQ ID NO: 406 |
| Cocadviroid MK795559.1 Hop latent viroid strain CV_40, complete genome | SEQ ID NO: 407 |
| Cocadviroid MK795550.1 Hop latent viroid strain CV_31, complete genome | SEQ ID NO: 408 |
| Cocadviroid MK795592.1 Hop latent viroid strain CV_73, complete genome | SEQ ID NO: 409 |
| Cocadviroid MK795632.1 Hop latent viroid strain CV_113, complete genome | SEQ ID NO: 410 |
| Cocadviroid MK795606.1 Hop latent viroid strain CV_87, complete genome | SEQ ID NO: 411 |
| Cocadviroid DL463096.1 Detection method of Hop latent virus, primer set and kit | SEQ ID NO: 412 |
| for detection of Hop latent virus | |
| Cocadviroid MK795569.1 Hop latent viroid strain CV_50, complete genome | SEQ ID NO: 413 |
| Cocadviroid MK795573.1 Hop latent viroid strain CV_54, complete genome | SEQ ID NO: 414 |
| Cocadviroid MK795566.1 Hop latent viroid strain CV_47, complete genome | SEQ ID NO: 415 |
| Cocadviroid MK791746.1 Hop latent viroid isolate CR3A, partial genome | SEQ ID NO: 416 |
| Cocadviroid MK795556.1 Hop latent viroid strain CV_37, complete genome | SEQ ID NO: 417 |
| Cocadviroid MK795623.1 Hop latent viroid strain CV_104, complete genome | SEQ ID NO: 418 |
| Cocadviroid KT964100.1 Coconut cadang-cadang viroid isolate RP1, partial | SEQ ID NO: 419 |
| genome | |
| Apscaviroid MK888699.1 Citrus dwarfing viroid isolate SL-06, complete genome | SEQ ID NO: 420 |
| Cocadviroid MK795574.1 Hop latent viroid strain CV_55, complete genome | SEQ ID NO: 421 |
| Cocadviroid MK795557.1 Hop latent viroid strain CV_38, complete genome | SEQ ID NO: 422 |
| Cocadviroid MK795554.1 Hop latent viroid strain CV_35, complete genome | SEQ ID NO: 423 |
| Cocadviroid MK795599.1 Hop latent viroid strain CV_80, complete genome | SEQ ID NO: 424 |
| Cocadviroid MK795560.1 Hop latent viroid strain CV_41, complete genome | SEQ ID NO: 425 |
| Apscaviroid MG879257.1 Citrus dwarfing viroid isolate E20-Cu-Ver, complete | SEQ ID NO: 426 |
| genome | |
| Apscaviroid JQ080280.1 Citrus bent leaf viroid isolate CBLVd-2, complete | SEQ ID NO: 427 |
| genome | |
| Cocadviroid MK795590.1 Hop latent viroid strain CV_71, complete genome | SEQ ID NO: 428 |
| Apscaviroid MG879255.1 Citrus dwarfing viroid isolate E14-SR-Ver, complete | SEQ ID NO: 429 |
| genome | |
| Cocadviroid MK795641.1 Hop latent viroid strain CV_122, complete genome | SEQ ID NO: 430 |
| Cocadviroid MK795611.1 Hop latent viroid strain CV_92, complete genome | SEQ ID NO: 431 |
| Cocadviroid MK795604.1 Hop latent viroid strain CV_85, complete genome | SEQ ID NO: 432 |
| Cocadviroid MK795644.1 Hop latent viroid strain CV_125, complete genome | SEQ ID NO: 433 |
| Avsunviroid AF404073.1 Avocado sunblotch viroid isolate CF59, complete | SEQ ID NO: 434 |
| genome | |
| Cocadviroid MK795531.1 Hop latent viroid strain CV_12, complete genome | SEQ ID NO: 435 |
| Apscaviroid JX892934.1 Grapevine yellow speckle viroid 1 clone 33 symptomatic, | SEQ ID NO: 436 |
| complete genome | |
| Cocadviroid MK795602.1 Hop latent viroid strain CV_83, complete genome | SEQ ID NO: 437 |
| Cocadviroid MK795578.1 Hop latent viroid strain CV_59, complete genome | SEQ ID NO: 438 |
| Cocadviroid MK795621.1 Hop latent viroid strain CV_102, complete genome | SEQ ID NO: 439 |
| Cocadviroid MK795552.1 Hop latent viroid strain CV_33, complete genome | SEQ ID NO: 440 |
| Apscaviroid LC427232.1 Citrus viroid VI persimmon, genomic RNA, partial | SEQ ID NO: 441 |
| sequence | |
| Apscaviroid JX892928.1 Grapevine yellow speckle viroid 1 clone 15 | SEQ ID NO: 442 |
| nonsymptomatic, complete genome | |
| Cocadviroid MK795598.1 Hop latent viroid strain CV_79, complete genome | SEQ ID NO: 443 |
| Cocadviroid MK795546.1 Hop latent viroid strain CV_27, complete genome | SEQ ID NO: 444 |
| Pelamoviroid JQ904596.1 Chrysanthemum chlorotic mottle viroid, partial | SEQ ID NO: 445 |
| sequence | |
| Cocadviroid MK795532.1 Hop latent viroid strain CV_13, complete genome | SEQ ID NO: 446 |
| Pospiviroid JX945671.1 Citrus exocortis viroid isolate E14, partial sequence | SEQ ID NO: 447 |
| Cocadviroid MK795582.1 Hop latent viroid strain CV_63, complete genome | SEQ ID NO: 448 |
| Apscaviroid MG879254.1 Citrus dwarfing viroid isolate E12-SR-Ver, complete | SEQ ID NO: 449 |
| genome | |
| Apscaviroid HE601745.1 Apple scar skin viroid partial genome, isolate Rohru | SEQ ID NO: 450 |
| Pospiviroid KP454039.1 Potato spindle tuber viroid isolate CVN259, partial | SEQ ID NO: 451 |
| sequence | |
| Cocadviroid MK795524.1 Hop latent viroid strain CV_5, complete genome | SEQ ID NO: 452 |
| Cocadviroid KC121568.1 Citrus bark cracking viroid isolate 6035, partial | SEQ ID NO: 453 |
| sequence | |
| Cocadviroid MK795575.1 Hop latent viroid strain CV_56, complete genome | SEQ ID NO: 454 |
| Cocadviroid MK795521.1 Hop latent viroid strain CV_2, complete genome | SEQ ID NO: 455 |
| Apscaviroid MG879258.1 Citrus dwarfing viroid isolate E21-AR-Ver, complete | SEQ ID NO: 456 |
| genome | |
| Apscaviroid MG879256.1 Citrus dwarfing viroid isolate E19-Cu-Ver, complete | SEQ ID NO: 457 |
| genome | |
| Pospiviroid MK330991.1 Potato spindle tuber viroid isolate 21819070-B sequence | SEQ ID NO: 458 |
| Pospiviroid DQ846884.1 Citrus exocortis viroid from Verbena × hybrida, partial | SEQ ID NO: 459 |
| genome | |
| Avsunviroid HC918585.1 Sequence 8 from Patent WO2010061186 | SEQ ID NO: 460 |
| Cocadviroid MF616006.1 Coconut cadang-cadang viroid isolate UPM 3-3 clone 2, | SEQ ID NO: 461 |
| partial genome | |
| Cocadviroid MF616002.1 Coconut cadang-cadang viroid isolate UPM 1-2 clone 2, | SEQ ID NO: 462 |
| partial genome | |
| Cocadviroid MF616004.1 Coconut cadang-cadang viroid isolate UPM 2-2 clone 2, | SEQ ID NO: 463 |
| partial genome | |
| Pelamoviroid FJ647358.1 Chrysanthemum chlorotic mottle viroid isolate plant 3 | SEQ ID NO: 464 |
| clone hammerhead 04, partial sequence | |
| Hostuviroid EU925587.1 Hop stunt viroid isolate 1, partial sequence | SEQ ID NO: 465 |
| Cocadviroid MF616001.1 Coconut cadang-cadang viroid isolate UPM 1-2 clone 1, | SEQ ID NO: 466 |
| partial genome | |
| Pospiviroid S52178.1 [potato spindle tuber viroid, Other Genetic Material RNA, 65 | SEQ ID NO: 467 |
| nt] | |

| | |
|---|---|
| Viroid | SEQ ID NO: 468, 469, 470, 471, 472, 473, 474, 475, 476, 477, 478, 479, 480, 481, 482, |
| consensus | 483, 484, 485, 486, 487, 488, 489, 490, 491, 492, 493, 494, 495, 496, 497, 498, 499, 500, |
| sequences | 501, 502, 503, 504, 505, 506, 507, 508, 509, 510, 511, 512, 513, 514, 515, 516, 517, 518, |
| | 519, 520, 521, 522, 523, 524, 525, 526, 527, 528, 529, 530, 531, 532, 533, 534, 535, 536, |
| | 537, 538, 539, 540, 541, 542, 543, 544, 545, 546, 547, 548, 549, 550, 551, 552, 553, 554, |
| | 555, 556, 557, 558, 559, 560, 561, 562, 563, 564, 565, 566, 567, 568, 569, 570, 571, 572, |
| | 573, 574, 575, 576, 577, 578, 579, 580, 581, 582, 583, 584, 585, 586, 587, 588, 589, 590, |
| | 591, 592, 593, 594, 595, 596, 597, 598, 599, 600, 601, 602, 603, 604, 605, 606, 607, 608, |
| | 609, 610, 611, 612, 613, 614, 615, 616, 617, 618, 619, 620, 621, 622, 623, 624, 625, 626, |
| | 627, 628, 629, 630, 631, 632, 633, 634, 635, 636, 637, 638, 639, 640, 641, 642, 643, 644, |
| | 645, 646, 647, 648, 649, 650, 651, 652, 653, 654, 655, 656, 657, 658, 659, 660, 661, 662, |
| | 663, 664, 665, 666, 667, 668, 669, 670, 671, 672, 673, 674, 675, 676, 677, 678, 679, 680, |
| | 681, 682, 683, 684, 685, 686, 687, 688, 689, 690, 691, 692, 693, 694, 695, 696, 697, 698, |
| | 699, 700, 701, 702, 703, 704, 705, 706, 707, 708, 709, 710, 711, 712, 713, 714, 715, 716, |
| | 717, 718, 719, 720, 721, 722, 723, 724, 725, 726, 727, 728, 729, 730, 731, 732, 733, 734, |
| | 735, 736, 737, 738, 739, 740, 741, 742, 743, 744, 745, 746, 747, 748, 749, 750, 751, 752, |
| | 753, 754, 755, 756, 757, 758, 759, 760, 761, 762, 763, 764, 765, 766, 767, 768, 769, 770, |
| | 771, 772, 773, 774, 775, 776, 777, 778, 779, 780, 781, 782, 783, 784, 785, 786, 787, 788, |
| | 789, 790, 791, 792, 793, 794, 795, 796, 797, 798, 799, 800, 801, 802, 803, 804, 805, 806, |
| | 807, 808, 809, 810, 811, 812, 813, 814, 815, 816, 817, 818, 819, 820, 821, 822, 823, 824, |
| | 825, 826, 827, 828, 829, 830, 831, 832, 833, 834, 835, 836, 837, 838, 839, 840, 841, 842, |
| | 843, 844, 845, 846, 847, 848, 849, 850, 851, 852, 853, 854, 855, 856, 857, 858, 859, 860, |
| | 861, 862, 863, 864, 865, 866, 867, 868, 869, 870, 871, 872, 873, 874, 875, 876, 877, 878, |
| | 879, 880, 881, 882, or 883. |

### B. Polynucleotides Comprising Viroid Domains

In some embodiments, the ssRNA viroid sequence comprises a sequence that is at least 80% identical to a sequence listed in Table 2 or Table 3, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a sequence listed in Table 2 or Table 3. In some embodiments, the ssRNA viroid sequence has at least 90% identity to a sequence of Table 2 or Table 3. In some embodiments, the ssRNA viroid sequence has at least 95% identity to a sequence of Table 2 or Table 3. In some embodiments, the ssRNA viroid sequence has at least 98% identity to a sequence of Table 2 or Table 3. In some embodiments, the ssRNA viroid sequence has at least 99% identity to a sequence of Table 2 or Table 3. In some embodiments, the ssRNA viroid sequence comprises or consists of a sequence listed in Table 2 or Table 3.

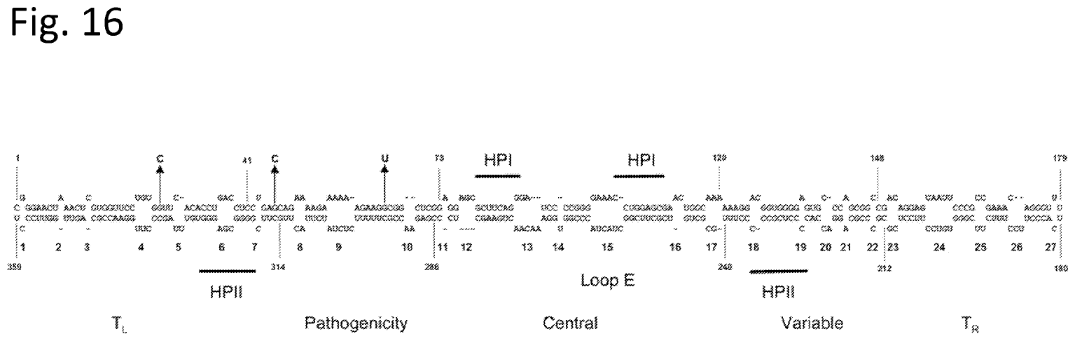

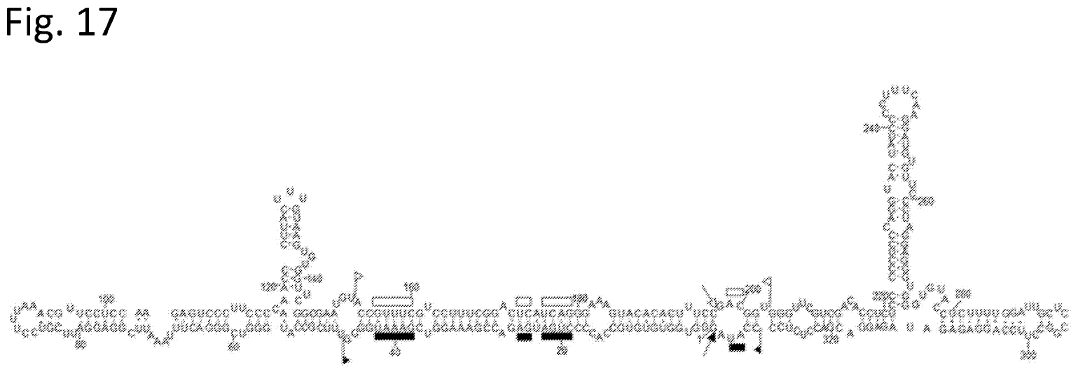

Table 2 shows the sequences of representative domains of PSTVd, and FIG. 16 shows the secondary structure of PSTVd. Table 3 shows the sequences of representative domains of ELVd, and FIG. 17 shows the secondary structure of ELVd.

In some embodiments, the recombinant polynucleotide encodes at least two ssRNA viroid sequences, and each of the at least two ssRNA viroid sequences is at least 80% identical to a sequence listed in Table 2 or Table 3. For example, in some embodiments, the ssRNA viroid sequence comprises a first sequence that is at least 80% identical to a sequence listed in Table 2 or Table 3, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a sequence listed in Table 2 or Table 3, and a second sequence that is at least 80% identical to a sequence listed in Table 2 or Table 3, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to a sequence listed in Table 2 or Table 3. In some embodiments, the recombinant polynucleotide comprises 3, 4, 5, 6, 7, 8, 9, 10, or more than 10 ssRNA viroid sequences that are each at least 80% identical to a sequence listed in Table 2 or Table 3. In some embodiments, the at least two ssRNA viroid sequences comprise at least one viroid sequence from each of at least two viroid genomes.

In some embodiments, the recombinant polynucleotide encodes a sequence that is at least 80% identical to SEQ ID NO: 884 and encodes a sequence that is at least 80% identical to SEQ ID NO: 885. For example, in some embodiments, the ssRNA viroid sequence comprises a first sequence that is at least 80% identical to SEQ ID NO: 884, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 884 and a second sequence that is at least 80% identical to SEQ ID NO: 885, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 885. In some embodiments, the sequence that is at least 80% identical to SEQ ID NO: 884 and the sequence that is at least 80% identical to SEQ ID NO: 885 base pair with one another, e.g., base pair with one another as shown in Table 2, e.g., base pair to form one or more loops.

In some embodiments, the one or more loops have a function relating to replication; initiation of transcription (e.g., Binding to TFIIIA 7ZF); or transmission (e.g., trafficking from palisade mesophyll to spongy mesophyll cells or vascular entry). In some embodiments, the recombinant polynucleotide comprises the left terminal domain (TL) of PSTVd.

In some embodiments, the recombinant polynucleotide encodes a sequence that is at least 80% identical to SEQ ID NO: 886 and encodes a sequence that is at least 80% identical to SEQ ID NO: 887. For example, in some embodiments, the ssRNA viroid sequence comprises a first sequence that is at least 80% identical to SEQ ID NO: 886, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 886 and a second sequence that is at least 80% identical to SEQ ID NO: 887, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 887. In some embodiments, the sequence that is at least 80% identical to SEQ ID NO: 886 and the sequence that is at least 80% identical to SEQ ID NO: 887 base pair with one another, e.g., base pair with one another as shown in Table 2, e.g., base pair to form one or more loops.

In some embodiments, the one or more loops have a function relating to pathogenicity. In some embodiments, the recombinant polynucleotide comprises the pathogenicity domain of PSTVd.

In some embodiments, the recombinant polynucleotide encodes a sequence that is at least 80% identical to SEQ ID NO: 888 and encodes a sequence that is at least 80% identical to SEQ ID NO: 889. For example, in some embodiments, the ssRNA viroid sequence comprises a first sequence that is at least 80% identical to SEQ ID NO: 888, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 888 and a second sequence that is at least 80% identical to SEQ ID NO: 889, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 889. In some embodiments, the sequence that is at least 80% identical to SEQ ID NO: 888 and the sequence that is at least 80% identical to SEQ ID NO: 889 base pair with one another, e.g., base pair with one another as shown in Table 2, e.g., base pair to form one or more loops.

In some embodiments, the one or more loops have a function relating to replication or alternative splicing (e.g., interacts with RPL5 to regulate alternative splicing for TF IIIA). In some embodiments, the recombinant polynucleotide comprises the central conserved domain of PSTVd.

In some embodiments, the recombinant polynucleotide encodes a sequence that is at least 80% identical to SEQ ID NO: 890 and encodes a sequence that is at least 80% identical to SEQ ID NO: 891. For example, in some embodiments, the ssRNA viroid sequence comprises a first sequence that is at least 80% identical to SEQ ID NO: 890, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 890 and a second sequence that is at least 80% identical to SEQ ID NO: 891, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 891. In some embodiments, the sequence that is at least 80% identical to SEQ ID NO: 890 and the sequence that is at least 80% identical to SEQ ID NO: 891 base pair with one another, e.g., base pair with one another as shown in Table 2, e.g., base pair to form one or more loops.

In some embodiments, the one or more loops have a function relating to transmission (e.g., trafficking from palisade mesophyll to spongy mesophyll cells). In some embodiments, the recombinant polynucleotide comprises the variable domain of PSTVd.

In some embodiments, the recombinant polynucleotide encodes a sequence that is at least 80% identical to SEQ ID NO: 892 and encodes a sequence that is at least 80% identical to SEQ ID NO: 893. For example, in some embodiments, the ssRNA viroid sequence comprises a first sequence that is at least 80% identical to SEQ ID NO: 892, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 892 and a second sequence that is at least 80% identical to SEQ ID NO: 893, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 893. In some embodiments, the sequence that is at least 80% identical to SEQ ID NO: 892 and the sequence that is at least 80% identical to SEQ ID NO: 893 base pair with one another, e.g., base pair with one another as shown in Table 2, e.g., base pair to form one or more loops.

In some embodiments, the one or more loops have a function relating to replication (e.g., comprise a TF IIIA 9ZF binding site, e.g., comprise a TF IIIA 9ZF binding site involved in systemic trafficking) or epidermal exit or comprise a systemic spread signal. In some embodiments, the recombinant polynucleotide comprises the right terminal domain (TR) of PSTVd.

In some embodiments, the recombinant polynucleotide encodes a sequence that is at least 80% identical to SEQ ID NO: 894, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 894. In some embodiments, the sequence that is at least 80% identical to SEQ ID NO: 894 base pairs with itself, e.g., base pairs with itself as shown in Table 3, e.g., base pairs to form one or more loops. In some embodiments, the one or more loops have a function relating to nuclear targeting, chloroplast targeting, or ribozyme activity (e.g., self-cleavage).

In some embodiments, the recombinant polynucleotide encodes a sequence that is at least 80% identical to SEQ ID NO: 895, e.g., is at least 80%, 81%, 82%, 83%, 84%, 85%, 86%, 87%, 88%, 89%, 90%, 91%, 92%, 93%, 94%, 95%, 96%, 97% 98%, 99%, or 100% identical to SEQ ID NO: 895. In some embodiments, the sequence that is at least 80% identical to SEQ ID NO: 895 base pairs with itself, e.g., base pairs with itself as shown in Table 3, e.g., base pairs to form one or more loops.

The dot bracket notation provided in Table 2 and Table 3 was generated using the RNA Fold software for predicting RNA secondary structure based on minimum free energy predictions of base pair probabilities.

A dot ‘.’ signifies an unpaired base and a bracket ‘(’ or ‘)’ represents a paired base. Dot bracket notation is further described in Mattei et al., Nucleic Acids Research, 42(10): 6146-6157, 2014; Ramlan and Zauner In International Workshop on Computing With Biomolecules, E. Csuhaj-Varju, R. Freund, M. Oswald and K. Salomaa (Eds.), 27 Aug. 2008, Wien, Austria, pp. 75-86, From: Austrian Computer Society, 2008; and Hofacker et al., Monatshelte Fur Chemie Chem. Monthly, 125: 167-188, 1994.

TABLE 2

| | Domain | Dot-Bracket | | | Nt | | Known | |
|---|---|---|---|---|---|---|---|---|
| Domain | sequence | Notation | Loop | Sequence | position | PVRD | Function | Host factors |
| Left | 1-41: | 5′- | 1 | 5′-CGG-3′ | 1-3 | Replication | Initiation of | RNA |
| Terminal | 5′CGGAACUAAA | .(((((((......))))) | | 3′-UCC-5′ | 359-357 | | transcription | polymerase II |
| Domain | CUCGUGGUUCC | ))...((((.......))) | | | | | | |
| (TL) | UGUGGUUCACA | )..-3′ | | | | | | |
| | CCUGACCUC3′ | 5′- | | | | | | |
| | (SEQ ID NO: 884) | (((....)))((((((... | 2 | UAA | 7-9 | | | |
| | 314-359: | ..))))))((((((..... | | G-U | 353-352 | | | |
| | 5′GCUUCGGGG | .)))))).-3′ | | | | | | |
| | CGAGGGUGUUU | | | | | | | |
| | AGCCCUUGGAA | | | | | | | |
| | CCGCAGUUGGU | | 3 | UCG | 12-14 | Replication | Binding to | TF IIIA 7ZF |
| | UCCU3′ | | | A-C | 349-348 | | TFIIIA 7ZF | |
| | (SEQ ID NO: 885) | | | | | | | |
| | | | 4 | CUGUG | 21-25 | Replication | essential for | |
| | | | | GUUCC | 341-337 | | initiation of | |
| | | | 5 | UC-A | 28-30 | Replication | transcription | |
| | | | | AUUU | 334-331 | | | |
| | | | TL- | CGGAACUA | 1-34 | Replication | | |
| | | | R | AACUCGUG | | | | |
| | | | | GUUCCUGU | | | | |
| | | | | GGUUCACA | | | | |
| | | | | CC | | | | |
| | | | | (SEQ ID NO: | 327-359 | | | |
| | | | | 896) | | | | |
| | | | | GGUGUUUA | | | | |
| | | | | GCCCUUGG | | | | |
| | | | | AACCGCAG | | | | |
| | | | | UUGGUUCC | | | | |
| | | | | U | | | | |
| | | | | (SEQ ID NO: | | | | |
| | | | | 897) | | | | |
| | | | 6 | UGACC | 35-39 | Transmission | Decisive | |
| | | | | GAGCG | 326-322 | motif | role in | |
| | | | | | | (palisade | PSTVd | |
| | | | | | | mesophyll to | trafficking | |
| | | | | | | spongy | from | |
| | | | | | | mesophyll | palisade | |
| | | | | | | cells) | mesophyll | |
| | | | | | | | to | |
| | | | | | | | spongy | |
| | | | | | | | mesophyll | |
| | | | | | | | cells of | |
| | | | | | | | tobacco | |
| | | | | | | | leaves. | |
| | | | 7 | CUG | 42-44 | Transmission | mediates | |
| | | | | GCU | 319-317 | motif | vascular | |
| | | | | | | (vascular | entry | |
| | | | | | | entry) | | |
| Patho- | 42-73: | 5′- | 8 | GAAA | 49-52 | | | |
| genicity | CUGAGCAGAAA | ((((((.............. | | UCAU | 312-309 | | | |
| Domain | AGAAAAAAGAA | ......)))))-3′ | | | | | | |
| | GGCGGCUCGG | | 9 | AAAAA-A | 55-60 | | | |
| | (SEQ ID NO: 886) | | | UAUCUCU | 306-300 | | | |
| | 286-313: | | 10 | G- -C | 68-69 | | | |
| | CGAGAACCGCU | | | CAAG | 292-289 | | | |
| | UUUUCUCUAUC | | | | | | | |
| | UUACUU | | | | | | | |
| | (SEQ ID NO: 887) | | | | | | | |
| Central | 74-120: | | 11 | GAG | 73-75 | | | |
| Conserved | AGGAGCGCUUC | | | C-C | 285-284 | | | |
| Domain | AGGGAUCCCCG | | 12 | GAGCG | 76-80 | | | |
| | GGGAAACCUGG | | | U---C | 283-282 | | | |
| | AGCGAACUGGC | | 13 | G-GGAU | 86-90 | Replication | replication | |
| | AAA | | | CAACAAA | 276-270 | | | |
| | (SEQ ID NO: 888) | | 14 | C-C | 92-93 | Replication | Regulate | |
| | 240-285: | | | GUG | 268-266 | | replication | |
| | UGCGCUGUCGC | | 15 | GGAAAC-C | 97-103 | Replication | Interacts | RPL5 |
| | UUCGGCUACUA | | | CAUCAUCG | 262-255 | | with RPL5 | |
| | CCCGGUGGAAA | | | | | | to regulate | |
| | CAACUGAAGCU | | | | | | alternative | |
| | CC | | | | | | splicing for | |
| | (SEQ ID NO: 889) | | | | | | TF IIIA | |
| | | | 16 | AACU | 111-114 | | | |
| | | | | U--G | 247-246 | | | |
| | | | 17 | CAAAA | 117-121 | | | |
| | | | | GCG-U | 243-240 | | | |
| Variable | 121-148: | 5′- | 18 | GACG | 125-128 | | | |
| Domain | AAAGGACGGUG | ...((.((.((((.....) | | CC-C | 236-234 | | | |
| | GGGAGUGCCCA | ))).)).))-3′ | | | | | | |
| | GCGGCC | 5′- | 19 | GAG | 134-136 | Transmission | Decisive | |
| | (SEQ ID NO: 890) | .....(((((......))) | | CCC | 228-226 | motif | role in | |
| | 212-239: | .))......-3′ | | | | (palisade | PSTVd | |
| | CGCCCGCAGGA | | | | | mesophyll to | trafficking | |
| | CCACCCCUCGC | | | | | spongy | from | |
| | CCCCUU | | | | | mesophyll | palisade | |
| | (SEQ ID NO: 891) | | | | | cells) | | |
| | | | | | | | mesophyll | |
| | | | | | | | to | |
| | | | | | | | spongy | |
| | | | | | | | mesophyll | |
| | | | | | | | cells of | |
| | | | | | | | tobacco | |
| | | | | | | | leaves. | |
| | | | 20 | GC-C | 138-140 | | | |
| | | | | CCAG | 224-221 | | | |
| | | | 21 | CAG | 141-143 | | | |
| | | | | GAC | 220-218 | | | |
| | | | 22 | GCC | 146-148 | | | |
| | | | | CCG | 215-213 | | | |
| Right | 149-179: | 5′- | 23 | GACA | 149-152 | | | |
| Terminal | GACAGGAGUAA | ....(((.....))).((( | | CGCU | 212-209 | | | |
| Domain | UUCCCGCCGAA | .......)))..-3′ | 24 | GUAAUUC | 156-162 | | | |
| (TR) | ACAGGGUUU | | | UCCUGUG | 205-199 | | | |
| | (SEQ ID NO: 892) | 5′- | 25 | GCCG | 165-168 | Replication | TF IIIA 9ZF | TF IIIA 9ZF |
| | 180-211: | .(((((...........)) | | CUUC | 196-193 | | binding site | VirP1 |
| | UCACCCUUCCU | )))..........-3′ | | | | | believed to | |
| | UUCUUCGGGUG | | | | | | be systemic | |
| | UCCUUCCUCG | | | | | | trafficking | |
| | (SEQ ID NO: 893) | | 26 | A -C- A | 171-173 | Transmission | Systemic | |
| | | | | UCCUU | 190-186 | motif | spread | |
| | | | | | | | signal | |
| | | | 27 | UUU | 177-179 | Transmission | PSTVd | |
| | | | | ACU | 182-180 | motif | RNA across | |
| | | | | | | (epidermal | the | |
| | | | | | | exit) | boundary | |
| | | | | | | | are unique | |
| | | | | | | | and | |
| | | | | | | | directional, | |
| | | | | | | | and the | |
| | | | | | | | U178G/ | |
| | | | | | | | U179G | |
| | | | | | | | deficiency | |
| | | | | | | | specifically | |
| | | | | | | | impacts | |
| | | | | | | | epidermal | |
| | | | | | | | exit | |

TABLE 3
Eggplant latent viroid (ELVd) domains

| | Dot- | | | | | | |
|---|---|---|---|---|---|---|---|
| | Bracket | | ELVd | | Nt | | |
| Sequence | Notation | Function | Loop | Sequence | position | PVRD | Function |
| 325-333-1-204: | 5′- | | 1 | UCCU | 85-88 | Targeting motif | Sufficient to mediate |
| CUCCCCAUAGGG | ....((...((( | | | UUAAA | 89-93 | (nucleus) | GFP mRNA into |
| UGGUGUGUGCCA | .(((((((((. | | | | | | nucleus (Gómez & |
| CCCCUGAUGAGA | ..((((((((( | | 2 | AUUC | 80-83 | | Pallas (2012) Plant |
| CCGAAAGGUCGA | (.((((((((. | | | GUU | 95-97 | | Physiol. 159:558- |
| AAUGGGGUUUCG | ((((((((... | | 3 | UAAAUUCG | 68-75 | | 564); |
| CCAUGGGUCGGG | ((((((..((( | | | CAA | 102-104 | | doi:10.1104/pp.112. |
| ACUUUAAAUUCG | ..((((((((.. | | 4 | GUCG | 58-61 | | 195214 |
| GAGGAUUCGUCC | ....((((((.. | | | CUUC | 111-114 | | |
| UUUAAACGUUCC | (((.......)) | | 5 | CAUG | 53-56 | | |
| UCCAAGAGUCCC | ).)))))).)) | | | CCAAACCCUUACUUUGU | 116-144 | | |
| UUCCCCAAACCCU | ))))))..))) | | | AAGUGUGGUUCG | | | |
| UACUUUGUAAGU | ..((((..(((( | Ribozyme | 6 | GGGUU | 44-48 | | |
| GUGGUUCGGCGA | (...))))).)) | | | AUGUAC | 149-154 | | |
| AUGUACCGUUUC | )).)))))).. | Ribozyme | 7 | GUC | 35-37 | | |
| GUCCUUUCGGAC | ..)))))))). | | | GUC | 161-163 | | |
| UCAUCAGGGAAA | )))))))).)) | Ribozyme | 8 | GAC | 26-28 | | |
| GUACACACUUUC | ))))))))... | | | GAC | 170-172 | | |
| CGACGGUG | ))))))))).) | Ribozyme | 9 | CCACC | 13-17 | | |
| (SEQ ID | ))...))..-3′ | | | GAAAG | 181-185 | | |
| NO: 894) | | Chloroplast | | ACCCCUGAUGAGACCGA | 15-181 | Targeting motif | (Gómez and Pallás, |
| | | targeting | | AAGGUCGAAAUGGGGU | | (chloroplast) | Plant Signal Behav, |
| | | | | UUCGCCAUGGGUCGGG | | | 5(11), 1517-1519, |
| | | | | ACUUUAAAUUCGGAGGA | | | 2010) |
| | | | | UUCGUCCUUUAAACGUU | | | |
| | | | | CCUCCAAGAGUCCCUUC | | | |
| | | | | CCCAAACCCUUACUUUG | | | |
| | | | | UAAGUGUGGUUCGGCG | | | |
| | | | | AAUGUACCGUUUCGUCC | | | |
| | | | | UUUCGGACUCAUCAGG | | | |
| | | | | G (SEQ ID NO: 900) | | | |
| | | Ribozyme | 10 | GUG | 3-5 | | |
| | | | | UUU | 193-195 | | |
| | | Ribozyme/ | 11 | CGACG | 197-201 | | |
| | | self- | | CCCAUAG | 328-1 | | |
| | | cleavage | | | | | |
| | | Ribozyme | 12 | GUG | 202-204 | | |
| | | | | CUC | 325-327 | | |
| 205-324 | 5′- | | 13 | GUU | 206-208 | | |
| GGUUCGUCGACA | (((.((((.(( | | | ACC | 321-323 | | |
| CCUCUCCCCCUC | ((((....(((( | | | | | | |
| CCAGGUACUAUC | ((.(((.(((( | | | | | | |
| CCCUUUCAAGGA | (((........) | | 14 | CGACACCUCUCCCCCUC | 212-279 | | |
| UGUGUUCCCUAG | )))).))..)) | | | | | | |
| GAGGGUGGGUGU | ).))))))).)) | | | CCAGGUACUAUCCCCUU | 310-317 | | |
| ACCUCUUUUGGA | ))))..((((( | | | UCAAGGAUGUGUUCCC | | | |
| UUGCUCCGGCCU | (((((..((... | | | UAGGAGGGUGGGUGUA | | | |
| UCCAGGAGAGAU | .))..)))))) | | | CC | | | |
| AGAGGACGACCU | ))))......)) | | | GAUAGAGG | | | |
| (SEQ ID | ))))).- 3′ | | 15 | AUUG | 288-291 | | |
| NO: 895) | | | | CCUU | 298-301 | | |
| | | | 16 | CUC | 292-294 | | |
| | | | | CGG | 295-297 | | |

i. Additional Heterologous Sequence Elements

In some aspects, the composition further comprises an additional sequence element that is heterologous to the viroid or heterologous to the viroid and the effector. In some aspects, the recombinant polynucleotide comprising: (1) a single-stranded RNA (ssRNA) viroid sequence and (ii) a heterologous RNA sequence comprising or encoding an effector further comprises an additional sequence element that is heterologous to the viroid or heterologous to the viroid and the effector. In embodiments, the additional heterologous sequence element is, e.g., an internal ribosome entry site (IRES: see Table 4), a 5 homology arm, a 3′ homology arm, a polyadenylation sequence, a group I permuted intron-exon (PIE) sequence, an RNA cleavage site, a ribozyme (e.g., a hammerhead ribozyme, a riboswitch, or a twister/tornado), a DICER-binding sequence (e.g., one or more DICER-binding sequences flanking the effector), an mRNA fragment comprising an intron, an exon, a combination of one or more introns and exons, an untranslated region (UTR), an enhancer region, a Kozak sequence, a start codon, or a linker.

TABLE 4
Internal ribosome entry sites (IRES)

| | | | Size | |
|---|---|---|---|---|
| IRES Class | Name | Sequence | (nt) | Reference |
| EMCV IRES | ECMV IRES-1 | SEQ ID NO: 901 | 463 | Urwin et al., The Plant Journal, |
| encephalomyocarditis | ECMV IRES-2 | SEQ ID NO: 902 | 552 | 24(5): 583-589, 2000. |
| virus | | | | |
| Maize hsp101 IRES | ZmHSP101 | SEQ ID NO: 903 | 150 | Jiménez-Gonzalez et al., PLoS |
| 5′UTR | IRES | | | ONE, 9(9): e107459, 2014. |
| crTMV CR, CP, 148 | Cr-TMV IRES | SEQ ID NO: 904 | 228 | Dorokhov et al., Proc Natl |
| (Crucifer infecting | | | | Acad Sci USA, 99(8): 5301- |
| tobamovirus) 5′ UTR | | | | 5306, 2002; Ivanov et al., |
| | | | | Virology, 232(1): 32-43, 1997. |
| Tobacco etch virus | TEV IRES | SEQ ID NO: 905 | 143 | Gallie, J. Virol., 75(24): 12141- |
| (TEV) IRES 5′UTR | | | | 12152, 2001; Bernet and |
| | | | | Elena, BMC Evol Biol, 15: 274, |
| | | | | 2015. |
| Hibiscus chlorotic | HCRSV IRES | SEQ ID NO: 906 | 100 | Koh et al., JBC, 278(23): |
| ringspot virus | | | | 20565-20573, 2003; Miller et |
| (HCRSV) | | | | al., Biochem Soc Trans, 35(6): |
| | | | | 1629-1633, 2007. |
| Classical swine | CSFV IRES | SEQ ID NO: 907 | 278 | Wesselhoeft et al., Nature |
| fever virus | | | | Communications, 9: Article No. |
| (CSFV) | | | | 2629, 2018; Friis et al., J Virol, |
| | | | | 86(16): 8681-8692, 2012. |
| coxsackievirus B3 | CVB3 IRES | SEQ ID NO: 908 | 208 | Wesselhoeft et al., Nature |
| virus (CVB3) | | | | Communications, 9: Article No. |
| | | | | 2629, 2018; Bhattacharyya et |
| | | | | al., Virology, 377(2): 435-354, |
| | | | | 2008; Liu et al., Virology: |
| | | | | 265(2): 206-217, 1999. |
| hepatitis A virus | HAV IRES | SEQ ID NO: 909 | 738 | Wesselhoeft et al., Nature |
| (HAV) | | | | Communications, 9: Article No. |
| | | | | 2629, 2018. |
| human rhinovirus | HTLV IRES | SEQ ID NO: 910 | 614 | Wesselhoeft et al., Nature |
| human T- | | | 449 | Communications, 9: Article No. |
| lymphotropic virus | | | | 2629, 2018; Olivares et al., J |
| (HTLV) | | | | Virol, 88(11): 5936-5955, |
| | | | | 2014. |
| Polyoma virus | PV (SV40) | SEQ ID NO: 911 | 742 | Wesselhoeft et al., Nature |
| (PV) | IRES | | | Communications, 9: Article No. |
| | | | | 2629, 2018; Yu and Alwine, J |
| | | | | Virol, 80(13): 6553-6558, |
| | | | | 2006. |

In some aspects, the composition or the recombinant polynucleotide comprising (I) a single-stranded RNA (ssRNA) viroid sequence and (ii) a heterologous RNA sequence comprising or encoding an effector further comprises 2, 3, 4, 5, 6, 7, 8, 9, 10, or more than 10 additional heterologous sequence elements, e.g., 2, 3, 4, 5, 6, 7, 8, 9, 10, or more than 10 of an internal ribosome entry site (IRES), a 5′ homology arm, a 3′ homology arm, a polyadenylation sequence, a group I permuted intron-exon (PIE) sequence, an RNA cleavage site, a ribozyme (e.g., a hammerhead ribozyme, a riboswitch, or a twister/tornado), a DICER-binding sequence (e.g., one or more DICER-binding sequences flanking the effector), an mRNA fragment comprising an intron, an exon, a combination of one or more introns and exons, an untranslated region (UTR), an enhancer region, a Kozak sequence, a start codon, or a linker. In some embodiments, the recombinant polynucleotide comprises more than one copy of an additional heterologous sequence element, e.g., comprises two, three, four, five, or more than five copies of such an element.

### C. Methods of Making Recombinant Polynucleotides

Recombinant polynucleotides comprising: (i) a single-stranded RNA (ssRNA) viroid sequence and (ii) a heterologous RNA sequence comprising or encoding an effector can be made using any method described herein or known in the art. In some embodiments, the recombinant polynucleotide is synthesized from a DNA template, e.g., using in vitro transcription, thus generating a linear recombinant polynucleotide. The recombinant polynucleotide ca be used in linear format (e.g., can be linear or linearized), or can be circularized or concatemeric. Methods for circularizing polynucleotides are described below.

Aspects of this disclosure are related to a composition comprising a double-stranded recombinant polynucleotide (e.g., a DNA) that is transcribed to produce a single-stranded recombinant polynucleotide of the disclosure, e.g., a single-stranded recombinant polynucleotide (e.g., an ssRNA) comprising: (i) a single-stranded RNA (ssRNA) viroid sequence and (ii) a heterologous RNA sequence comprising or encoding an effector. The double-stranded polynucleotide can be transcribed in vitro or in vivo, e.g., manufactured in a host cell (e.g., a bacterial cell) or translated in a plant cell, an arthropod cell, a mollusk cell, a fungal cell, or a nematode cell. In some aspects, the cell has been transiently transformed or stably transformed with the double-stranded recombinant polynucleotide (e.g., DNA).

### Circular Recombinant Polynucleotides

In some aspects, the recombinant polynucleotide comprising (i) a single-stranded RNA (ssRNA) viroid sequence and (ii) a heterologous RNA sequence comprising or encoding an effector is circular, e.g., has no free ends. Circular RNA is more resistant to exonuclease degradation than linear RNA due to the lack of 5′ and 3 ends. Circular recombinant polynucleotides can be produced using several methods, as described herein.

In some embodiments, a linear recombinant polynucleotide (e.g., RNA) is circularized using splint ligation. Splint DNA is designed to anneal between about 5 and 25 nucleotides (nt) of each 5′ or 3′ end of the linear polynucleotide (e.g., RNA) (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nt), leaving 2 nt at each end of the polynucleotide (e.g., RNA) unpaired. The linear recombinant polynucleotide (e.g., RNA) is annealed with the splint DNA and incubated with a ligase (e.g., T4 RNA ligase 2).

In some embodiments, a linear recombinant polynucleotide (e.g., RNA) is circularized using a tRNA ligase. Viroids are plant pathogens consisting of a single-stranded circular RNA that replicate in host cells and are circularized by endogenous tRNA ligases. In some embodiments, linear recombinant polynucleotides (e.g., RNAs) are circularized in a bacterial cell, e.g., an E. coli cell, in which an appropriate tRNA ligase (e.g., an eggplant tRNA ligase) is present.

In some embodiments, a linear recombinant polynucleotide (e.g., RNA) is circularized using ligation of ribozyme-cleaved ends. Ribozyme-cleaved ends of linear RNAs can be joined to synthesize circular RNA, e.g., as described in Litke and Jaffrey, Nature Biotechnology, 37: 667-675, 2019. Recently described “Twister” ribozymes undergo self-cleavage to produce 5′ hydroxyl and 2′,3′-cylic phosphate ends. These ends are recognized for ligation by the E. coli RNA ligase RtcB. To trigger RNA circularization, RNA transcripts are expressed containing an RNA of interest flanked by ribozymes that undergo spontaneous autocatalytic cleavage. The resulting RNA contains 5′ and 3′ ends that are then ligated by the nearly ubiquitous endogenous RNA ligase RtcB, thereby producing circular RNAs. Circularization of a polynucleotide can be detected, e.g., using denaturing polyacrylamide gel electrophoresis (PAGE). Because of their circular structure, circular polynucleotides (e.g., RNAs) migrate more slowly than linear polynucleotides (e.g., RNAs) on PAGE gels. Additionally, circularization of a polynucleotide (e.g., RNA) can be assessed using digestion with RNAse H, a nonspecific endonuclease that recognizes DNA/RNA duplexes. For a linear RNA, it is expected that after binding of the DNA oligomer and subsequent cleavage by RNAse H, two cleavage products are obtained. A concatemer is expected to produce at least three cleavage products. A circular RNA is expected to produce a single cleavage product. This is visualized as the presence of one, two or three bands on a gel.

### D. Formulations of Recombinant Polynucleotides

The compositions described herein can be formulated either in pure form (e.g., the composition contains only the recombinant polynucleotide) or together with one or more additional formulation components to facilitate application or delivery of the compositions. In embodiments, the additional formulation component includes, e.g., a carrier (i.e., a component that has an active role in delivering the active agent (e.g., recombinant polynucleotide); for example, a carrier can encapsulate, covalently or non-covalently modify, or otherwise associate with the active agent in a manner that improves delivery of the active agent) or an excipient (e.g., a delivery vehicle, adjuvant, diluent, surfactant, stabilizer, or tonicity agent).

In some embodiments, the composition is formulated for delivery to a plant.

In some aspects, the disclosure provides a formulation comprising any of the compositions described herein. In some embodiments, the formulation is a liquid, a gel, or a powder. In some embodiments, the formulation is configured to be sprayed on plants, to be injected into plants, to be rubbed on leaves, to be soaked into plants, to be coated onto plants, or be coated on seeds, or to be delivered through root uptake (e.g., in a hydroponic system or via soil).

Depending on the intended objectives and prevailing circumstances, the composition can be formulated into emulsifiable concentrates, suspension concentrates, directly sprayable or dilutable solutions, coatable pastes, diluted emulsions, spray powders, soluble powders, dispersible powders, wettable powders, dusts, granules, encapsulations in polymeric substances, microcapsules, foams, aerosols, carbon dioxide gas preparations, tablets, resin preparations, paper preparations, nonwoven fabric preparations, or knitted or woven fabric preparations. In some instances, the composition is a liquid. In some instances, the composition is a solid. In some instances, the composition is an aerosol, such as in a pressurized aerosol can.

In some instances, the recombinant polynucleotide makes up about 0.1% to about 100% of the composition, such as any one of about 0.01% to about 100%, about 1% to about 99.9%, about 0.1% to about 10%, about 1% to about 25%, about 10% to about 50%, about 50% to about 99%, or about 0.1% to about 90% of active ingredients (e.g., recombinant polynucleotides). In some instances, the composition includes at least any of 0.1%, 0.5%, 1%, 5%, 10%, 20%, 30%, 40%, 50%, 60%, 70%, 80%, 90%, 95%, or more active ingredients (e.g., recombinant polynucleotides). In some instances, the concentrated agents are preferred as commercial products, the final user normally uses diluted agents, which have a substantially lower concentration of active ingredient.

i. Formulation for Topical Delivery

In some embodiments, the composition is formulated for topical delivery to a plant. In some embodiments, the topical delivery is spraying, leaf rubbing, soaking, coating (e.g., coating using micro-particulates or nano-particulates), or delivery through root uptake (e.g., delivery in a hydroponic system).

In some embodiments, the composition further comprises a carrier and/or an excipient. In other embodiments, the composition does not comprise a carrier or excipient, e.g., comprises a naked polynucleotide (e.g., a naked RNA).

In some embodiments, the recombinant polynucleotide is delivered at a concentration of at least 0.1 grams per acre, e.g., at least 0.1, 1, 2, 5, 10, 15, 20, 25, 30, 35, 40, 45, or 50 grams per acre. In some embodiments, less than 120 liters per acre is delivered, e.g., less than 110, 100, 90, 80, 70, 60, 50, 40, 30, 20, 15, 10, 5, or 2 liters per acre or less than 1 liter per acre.

ii. Carriers

In some aspects, the formulation comprises a carrier. In some embodiments the formulation is an emulsion or a reverse emulsion, a liquid, or a gel. In embodiments, the formulation includes a carrier that serves as a physical support (e.g., solid or semi-solid surfaces or matrices, powders, or particles or nanoparticles). In embodiments, the active agent is encapsulated or enclosed in or attached to or complexed with a carrier including a liposome, vesicle, micelle, or other fluid compartment. In embodiments, the active agent is encapsulated or enclosed in or attached to or complexed with a carrier including a naturally occurring or synthetic, branched or linear polymer (e.g., pectin, agarose, chitin, chitosan, DEAE-dextran, polyvinylpyrrolidone (“PVP”), or polyethylenimine (“PEI”)). In embodiments the carrier includes cations or a cationic charge, such as cationic liposomes or cationic polymers such as polyamines (e.g., spermine, spermidine, putrescine). In embodiments, the carrier includes a polypeptide such as an enzyme, (e.g., cellulase, pectolyase, maceroenzyme, pectinase), a cell penetrating or pore-forming peptide (e.g., poly-lysine, poly-arginine, or polyhomoarginine peptides).

Non-limiting examples of carriers include cationic liposomes and polymer nanoparticles reviewed by Zhang et al. (2007) J. Controlled Release, 123:1-10, and the cross-linked multilamellar liposomes described in US Patent Application Publication 2014/0356414 AI, incorporated by reference in its entirety herein. In embodiments, the carrier includes a nanomaterial, such as carbon or silica nanoparticles, carbon nanotubes, carbon nanofibers, or carbon quantum dots. Non-limiting examples of carriers include particles or nanoparticles (e.g., particles or nanopartices made of materials such as carbon, silicon, silicon carbide, gold, tungsten, polymers, or ceramics) in various size ranges and shapes, magnetic particles or nanoparticles (e.g., silenceMag Magnetotransfection™ agent, OZ Biosciences, San Diego, CA), abrasive or scarifying agents, needles or microneedles, matrices, and grids. In certain embodiments, particulates and nanoparticulates are useful in delivery of the polynucleotide composition or the nuclease or both. Useful particulates and nanoparticles include those made of metals (e.g., gold, silver, tungsten, iron, cerium), ceramics (e.g., aluminum oxide, silicon carbide, silicon nitride, tungsten carbide), polymers (e.g., polystyrene, polydiacetylene, and poly(3,4-ethylenedioxythiophene) hydrate), semiconductors (e.g., quantum dots), silicon (e.g., silicon carbide), carbon (e.g., graphite, graphene, graphene oxide, or carbon nanosheets, nanocomplexes, or nanotubes), and composites (e.g., polyvinylcarbazole/graphene, polystyrene/graphene, platinum/graphene, palladium/graphene nanocomposites). In certain embodiments, such particulates and nanoparticulates are further covalently or non-covalently functionalized, or further include modifiers or cross-linked materials such as polymers (e.g., linear or branched polyethylenimine, poly-lysine), polynucleotides (e.g., DNA or RNA), polysaccharides, lipids, polyglycols (e.g., polyethylene glycol, thiolated polyethylene glycol), polypeptides or proteins, and detectable labels (e.g., a fluorophore, an antigen, an antibody, or a quantum dot). In various embodiments, such particulates and nanoparticles are neutral, or carry a positive charge, or carry a negative charge. Embodiments of compositions including particulates include those formulated, e.g., as liquids, colloids, dispersions, suspensions, aerosols, gels, and solids. Embodiments include nanoparticles affixed to a surface or support, e.g., an array of carbon nanotubes vertically aligned on a silicon or copper wafer substrate. Embodiments include polynucleotide compositions including particulates (e.g., gold or tungsten or magnetic particles) delivered by a Biolistic-type technique or with magnetic force. The size of the particles used in Biolistics is generally in the “microparticle” range, for example, gold microcarriers in the 0.6, 1.0, and 1.6 micrometer size ranges (see, e.g., instruction manual for the Helios® Gene Gun System, Bio-Rad, Hercules, CA; Randolph-Anderson et al. (2015) “Submicron gold particles are superior to larger particles for efficient Biolistic® transformation of organelles and some cell types”, Bio-Rad US/EG Bulletin 2015), but successful Biolistics delivery using larger (40-48—WO 2019/144124 PCT/0S2019/014559 nanometer) nanoparticles has been reported in cultured animal cells; see O'Brian and Lummis (2011) BMC Biotechnol., 11:66-71. Other embodiments of useful particulates are nanoparticles, which are generally in the nanometer (nm) size range or less than 1 micrometer, e.g., with a diameter of less than about 1 nm, less than about 3 nm, less than about 5 nm, less than about 10 nm, less than about 20 nm, less than about 40 nm, less than about 60 nm, less than about 80 nm, and less than about 100 nm. Specific, non-limiting embodiments of nanoparticles commercially available (all from Sigma-Aldrich Corp., St. Louis, MO) include gold nanoparticles with diameters of 5, 10, or 15 nm; silver nanoparticles with particle sizes of 10, 20, 40, 60, or 100 nm; palladium “nanopowder” of less than 25 nm particle size; single-, double-, and multi-walled carbon nanotubes, e.g., with diameters of 0.7-1.1, 1.3-2.3, 0.7-0.9, or 0.7-1.3 nm, or with nano tube bundle dimensions of 2-10 nm by 1-5 micrometers, 6-9 nm by 5 micrometers, 7-15 nm by 0.5-10 micrometers, 7-12 nm by 0.5-10 micrometers, 110-170 nm by 5-9 micrometers, 6-13 nm by 2.5-20 micrometers. Embodiments include polynucleotide compositions including materials such as gold, silicon, cerium, or carbon, e.g., gold or gold-coated nanoparticles, silicon carbide whiskers, carborundum, porous silica nanoparticles, gelatin/silica nanoparticles, nanoceria or cerium oxide nanoparticles (CNPs), carbon nanotubes (CNTs) such as single-, double-, or multi-walled carbon nanotubes and their chemically functionalized versions (e.g., carbon nanotubes functionalized with amide, amino, carboxylic acid, sulfonic acid, or polyethylene glycol moeities), and graphene or graphene oxide or graphene complexes; see, for example, Wong et al. (2016) Nano Lett., 16: 1161-1172; Giraldo et al. (2014) Nature Materials, 13:400-409; Shen et al. (2012) Theranostics, 2:283-294; Kim et al. (2011) Bioconjugate Chem., 22:2558-2567; Wang et al. (2010) J. Am. Chem. Soc. Comm., 132:9274-9276; Zhao et al. (2016) Nanoscale Res. Lett., 11: 195-203; and Choi et al. (2016) J. Controlled Release, 235:222-235. See also, for example, the various types of particles and nanoparticles, their preparation, and methods for their use, e.g., in delivering polynucleotides and polypeptides to cells, disclosed in US Patent Application Publications 2010/0311168, 2012/0023619, 2012/0244569, 2013/0145488, 2013/0185823, 2014/0096284, 2015/0040268, 2015/0047074, and 2015/0208663, all of which are incorporated herein by reference in their entirety.

iii. Excipients

In some aspects, the composition includes an excipient, e.g., a delivery vehicle, adjuvant, diluent, surfactant, stabilizer, or tonicity agent or a combination thereof. In some embodiments, the excipient is a crop oil concentrate, a vegetable oil concentrate, a modified vegetable oil, a nitrogen source, a deposition (drift control) and/or retention agent (with or without ammonium sulfate and/or defoamer), a compatibility agent, a buffering agent and/or acidifier, a water conditioning agent, a basic blend, a spreader-sticker and/or extender, an adjuvant plus foliar fertilizer, an antifoam agent, a foam marker, a scent, or a tank cleaner and/or neutralizer. In some embodiments, the excipient is an adjuvant described in the Compendium of Herbicide Adjuvants (Young et al. (2016). Compendium of Herbicide Adjuvants (13^{th} ed.), Purdue University).

Examples of delivery vehicles and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. Further exemplary delivery vehicles include, but are not limited to, solid or liquid excipient materials, solvents, stabilizers, slow-release excipients, colorings, and surface-active substances (surfactants). In some instances, the excipient (e.g., delivery vehicle) is a stabilizing vehicle. In embodiments, the stabilizing vehicle includes, e.g., an epoxidized vegetable oil, an antifoaming agent, e.g. silicone oil, a preservative, a viscosity regulator, a binding agent, or a tackifier. In some instances, the stabilizing vehicle is a buffer suitable for the recombinant polynucleotide. In some instances, the composition is microencapsulated in a polymer bead delivery vehicle. In some instances, the stabilizing vehicle protects the recombinant polynucleotide against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

iv. Adjuvants

In some instances, the composition provided herein includes an adjuvant. Adjuvants are agents that do not possess effector activity, but impart beneficial properties to a formulation. For example, adjuvants are either pre-mixed in the formulation or added to a spray tank to improve mixing or application or to enhance performance. They are used extensively in products designed for foliar applications. Adjuvants can be used to customize the formulation to specific needs and compensate for local conditions. Adjuvants can be designed to perform specific functions, including wetting, spreading, sticking, reducing evaporation, reducing volatilization, buffering, emulsifying, dispersing, reducing spray drift, and reducing foaming. No single adjuvant can perform all these functions, but compatible adjuvants often can be combined to perform multiple functions simultaneously.

Among nonlimiting examples of adjuvants included in the formulation are binders, dispersants and stabilizers, specifically, for example, casein, gelatin, polysaccharides (e.g., starch, gum arabic, cellulose derivatives, alginic acid, etc.), lignin derivatives, bentonite, sugars, synthetic water-soluble polymers (e.g., polyvinyl alcohol, polyvinylpyrrolidone, polyacrylic acid, etc.), PAP (acidic isopropyl phosphate), BHT (2,6-di-t-butyl-4-methylphenol), BHA (a mixture of 2-t-butyl-4-methoxyphenol and 3-t-butyl-4-methoxyphenol), vegetable oils, mineral oils, fatty acids and fatty acid esters.

v. Liquid and Gaseous Formulations

In embodiments, the compositions provided herein are in a liquid formulation. Liquid formulations are generally mixed with water, but in some instances are used with crop oil, diesel fuel, kerosene or other light oil as an excipient. The amount of active ingredient (e.g., recombinant polynucleotides) often ranges from about 0.5 to about 80 percent by weight.

In embodiments, an emulsifiable concentrate formulation contains a liquid active ingredient, one or more petroleum-based solvents, and an agent that allows the formulation to be mixed with water to form an emulsion. Such concentrates can be used in agricultural, ornamental and turf, forestry, structural, food processing, livestock, and public health pest formulations. In embodiments, these are adaptable to application equipment from small portable sprayers to hydraulic sprayers, low-volume ground sprayers, mist blowers, and low-volume aircraft sprayers. Some active ingredients readily dissolve in a liquid excipient. When mixed with an excipient, they form a solution that does not settle out or separate, e.g., a homogenous solution. In embodiments, formulations of these types include an active ingredient, a carrier and/or an excipient, and one or more other ingredients. Solutions can be used in any type of sprayer, indoors and outdoors.

In some instances, the composition is formulated as an invert emulsion. An invert emulsion is a water-soluble active ingredient dispersed in an oil excipient. Invert emulsions require an emulsifier that allows the active ingredient to be mixed with a large volume of petroleum-based excipient, usually fuel oil. Invert emulsions aid in reducing drift. With other formulations, some spray drift results when water droplets begin to evaporate before reaching target surfaces; as a result the droplets become very small and lightweight. Because oil evaporates more slowly than water, invert emulsion droplets shrink less and more active ingredient reaches the target. Oil further helps to reduce runoff and improve rain resistance. It further serves as a sticker-spreader by improving surface coverage and absorption. Because droplets are relatively large and heavy, it is difficult to get thorough coverage on the undersides of foliage. Invert emulsions are most commonly used along rights-of-way where drift to susceptible non-target areas can be a problem.

A flowable or liquid formulation combines many of the characteristics of emulsifiable concentrates and wettable powders. Manufacturers use these formulations when the active ingredient is a solid that does not dissolve in either water or oil. The active ingredient, impregnated on a substance such as clay, is ground to a very fine powder. The powder is then suspended in a small amount of liquid. The resulting liquid product is quite thick. Flowables and liquids share many of the features of emulsifiable concentrates, and they have similar disadvantages. They require moderate agitation to keep them in suspension and leave visible residues, similar to those of wettable powders.

Flowables/liquids are easy to handle and apply. Because they are liquids, they are subject to spilling and splashing. They contain solid particles, so they contribute to abrasive wear of nozzles and pumps.

Flowable and liquid suspensions settle out in their containers. Because flowable and liquid formulations tend to settle, packaging in containers of five gallons or less makes remixing easier.

Aerosol formulations contain one or more active ingredients and a solvent. Most aerosols contain a low percentage of active ingredients. There are two types of aerosol formulations—the ready-to-use type commonly available in pressurized sealed containers and those products used in electrical or gasoline-powered aerosol generators that release the formulation as a smoke or fog.

Ready to use aerosol formulations are usually small, self-contained units that release the formulation when the nozzle valve is triggered. The formulation is driven through a fine opening by an inert gas under pressure, creating fine droplets. These products are used in greenhouses, in small areas inside buildings, or in localized outdoor areas. Commercial models, which hold five to 5 pounds of active ingredient, are usually refillable.

Smoke or fog aerosol formulations are not under pressure. They are used in machines that break the liquid formulation into a fine mist or fog (aerosol) using a rapidly whirling disk or heated surface.

In some embodiments, the composition comprises a liquid excipient. In embodiments, a liquid excipient includes, for example, aromatic or aliphatic hydrocarbons (e.g., xylene, toluene, alkylnaphthalene, phenylxylylethane, kerosene, gas oil, hexane, cyclohexane, etc.), halogenated hydrocarbons (e.g., chlorobenzene, dichloromethane, dichloroethane, trichloroethane, etc.), alcohols (e.g., methanol, ethanol, isopropyl alcohol, butanol, hexanol, benzyl alcohol, ethylene glycol, etc.), ethers (e.g., diethyl ether, ethylene glycol dimethyl ether, diethylene glycol monomethyl ether, diethylene glycol monoethyl ether, propylene glycol monomethyl ether, tetrahydrofuran, dioxane, etc.), esters (e.g., ethyl acetate, butyl acetate, etc.), ketones (e.g., acetone, methyl ethyl ketone, methyl isobutyl ketone, cyclohexanone, etc.), nitriles (e.g., acetonitrile, isobutyronitrile, etc.), sulfoxides (e.g., dimethyl sulfoxide, etc.), amides (e.g., N,N-dimethylformamide, N,N-dimethylacetamide, cyclic imides (e.g. N-methylpyrrolidone) alkylidene carbonates (e.g., propylene carbonate, etc.), vegetable oil (e.g., soybean oil, cottonseed oil, etc.), vegetable essential oils (e.g., orange oil, hyssop oil, lemon oil, etc.), or water.

In some embodiments, the composition comprises a gaseous excipient. Gaseous excipients include, for example, butane gas, flon gas, liquefied petroleum gas (LPG), dimethyl ether, and carbon dioxide gas.

vi. Dry or Solid Formulations

Dry formulations can be divided into two types: ready-to-use and concentrates that must be mixed with water to be applied as a spray. Most dust formulations are ready to use and contain a low percentage of active ingredients (less than about 10 percent by weight), plus a very fine, dry inert excipient made from talc, chalk, clay, nut hulls, or volcanic ash. The size of individual dust particles varies. A few dust formulations are concentrates and contain a high percentage of active ingredients. Mix these with dry inert excipients before applying. Dusts are always used dry and can easily drift to non-target sites.

vii. Granule or Pellet Formulations

In some instances, the composition is formulated as granules. Granular formulations are similar to dust formulations, except granular particles are larger and heavier. In embodiments, the coarse particles are made from materials such as clay, corncobs, or walnut shells. The active ingredient either coats the outside of the granules or is absorbed into them. In embodiments, the amount of active ingredient is relatively low, usually ranging from about 0.5 to about 15 percent by weight. Granular formulations are most often used to apply to the soil, insects or nematodes living in the soil, or absorption into plants through the roots. Granular formulations are sometimes applied by airplane or helicopter to minimize drift or to penetrate dense vegetation. Once applied, granules can release the active ingredient slowly. Some granules require soil moisture to release the active ingredient. Granular formulations also are used to control larval mosquitoes and other aquatic pests. Granules are used in agricultural, structural, ornamental, turf, aquatic, right-of-way, and public health (biting insect) pest-control operations.

In some instances, the composition is formulated as pellets. Most pellet formulations are very similar to granular formulations; the terms are used interchangeably. In a pellet formulation, however, all the particles are the same weight and shape. The uniformity of the particles allows use with precision application equipment.

viii. Powders

In some instances, the composition is formulated as a powder. In some instances, the composition is formulated as a wettable powder. Wettable powders are dry, finely ground formulations that look like dusts. They usually must be mixed with water for application as a spray. A few products, however, can be applied either as a dust or as a wettable powder—the choice is left to the applicator. Wettable powders have about 1 to about 95 percent active ingredient by weight; in some cases more than about 50 percent. The particles do not dissolve in water. They settle out quickly unless constantly agitated to keep them suspended. They can be used for most pest problems and in most types of spray equipment where agitation is possible. Wettable powders have excellent residual activity. Because of their physical properties, most of the formulation remains on the surface of treated porous materials such as concrete, plaster, and untreated wood. In such cases, only the water penetrates the material.

In some instances, the composition is formulated as a soluble powder. Soluble powder formulations look like wettable powders. However, when mixed with water, soluble powders dissolve readily and form a true solution. After they are mixed thoroughly, no additional agitation is necessary. The amount of active ingredient in soluble powders ranges from about 15 to about 95 percent by weight; in some cases more than about 50 percent. Soluble powders have all the advantages of wettable powders and none of the disadvantages, except the inhalation hazard during mixing.

In some instances, the composition is formulated as a water-dispersible granule. Water-dispersible granules, also known as dry flowables, are like wettable powders, except instead of being dust-like, they are formulated as small, easily measured granules. Water-dispersible granules must be mixed with water to be applied. Once in water, the granules break apart into fine particles similar to wettable powders. The formulation requires constant agitation to keep it suspended in water. The percentage of active ingredient is high, often as much as 90 percent by weight. Water-dispersible granules share many of the same advantages and disadvantages of wettable powders, except they are more easily measured and mixed. Because of low dust, they cause less inhalation hazard to the applicator during handling In some embodiments, the composition comprises a solid excipient. Solid excipients include finely-divided powder or granules of clay (e.g. kaolin clay, diatomaceous earth, bentonite, Fubasami clay, acid clay, etc.), synthetic hydrated silicon oxide, talc, ceramics, other inorganic minerals (e.g., sericite, quartz, sulfur, activated carbon, calcium carbonate, hydrated silica, etc.), a substance which can be sublimated and is in the solid form at room temperature (e.g., 2,4,6-triisopropyl-1,3,5-trioxane, naphthalene, p-dichlorobenzene, camphor, adamantan, etc.); wool; silk; cotton; hemp; pulp; synthetic resins (e.g., polyethylene resins such as low-density polyethylene, straight low-density polyethylene and high-density polyethylene; ethylene-vinyl ester copolymers such as ethylene-vinyl acetate copolymers; ethylene-methacrylic acid ester copolymers such as ethylene-methyl methacrylate copolymers and ethylene-ethyl methacrylate copolymers; ethylene-acrylic acid ester copolymers such as ethylene-methyl acrylate copolymers and ethylene-ethyl acrylate copolymers; ethylene-vinylcarboxylic acid copolymers such as ethylene-acrylic acid copolymers; ethylene-tetracyclododecene copolymers; polypropylene resins such as propylene homopolymers and propylene-ethylene copolymers; poly-4-methylpentene-1, polybutene-1, polybutadiene, polystyrene; acrylonitrile-styrene resins; styrene elastomers such as acrylonitrile-butadiene-styrene resins, styrene-conjugated diene block copolymers, and styrene-conjugated diene block copolymer hydrides; fluororesins; acrylic resins such as poly(methyl methacrylate); polyamide resins such as nylon 6 and nylon 66; polyester resins such as polyethylene terephthalate, polyethylene naphthalate, polybutylene terephthalate, and polycyclohexylenedimethylene terephthalate; polycarbonates, polyacetals, polyacrylsulfones, polyarylates, hydroxybenzoic acid polyesters, polyetherimides, polyester carbonates, polyphenylene ether resins, polyvinyl chloride, polyvinylidene chloride, polyurethane, and porous resins such as foamed polyurethane, foamed polypropylene, or foamed ethylene, etc.), glasses, metals, ceramics, fibers, cloths, knitted fabrics, sheets, papers, yarn, foam, porous substances, and multifilaments.

ix. Nanocapsules/Microencapsulation/Liposomes

In some instances, the composition is provided in a microencapsulated formulation. Microencapsulated formulations are mixed with water and sprayed in the same manner as other sprayable formulations.

After spraying, the plastic coating breaks down and slowly releases the active ingredient.

In some instances, the composition is provided in a liposome. In some instances, the composition is provided in a vesicle.

x. Surfactants

In some instances, the composition provided herein includes a surfactant. Surfactants, also called wetting agents and spreaders, physically alter the surface tension of a spray droplet. For a formulation to perform its function property, a spray droplet must be able to wet the foliage and spread out evenly over a leaf. Surfactants enlarge the area of formulation coverage, thereby increasing exposure to the active agent. Surfactants are particularly important when applying a formulation to waxy or hairy leaves. Without proper wetting and spreading, spray droplets often run off or fail to cover leaf surfaces adequately. Too much surfactant, however, can cause excessive runoff and reduce efficacy.

Surfactants are classified by the way they ionize or split apart into electrically charged atoms or molecules called ions. A surfactant with a negative charge is anionic. One with a positive charge is cationic, and one with no electrical charge is nonionic. Formulation activity in the presence of a nonionic surfactant can be quite different from activity in the presence of a cationic or anionic surfactant. Selecting the wrong surfactant can reduce the efficacy of a product and injure the target plant. Anionic surfactants are most effective when used with contact pesticides (pesticides that control a pest by direct contact rather than being absorbed systemically). Cationic surfactants should never be used as stand-alone surfactants because they usually are phytotoxic.

Nonionic surfactants, often used with systemic pesticides, help sprays penetrate plant cuticles. Nonionic surfactants are compatible with most pesticides, and most EPA-registered pesticides that require a surfactant recommend a nonionic type. Adjuvants include, but are not limited to, stickers, extenders, plant penetrants, compatibility agents, buffers or pH modifiers, drift control additives, defoaming agents, and thickeners.

Among nonlimiting examples of surfactants included in the compositions described herein are alkyl sulfate ester salts, alkyl sulfonates, alkyl aryl sulfonates, alkyl aryl ethers and polyoxyethylenated products thereof, polyethylene glycol ethers, polyvalent alcohol esters and sugar alcohol derivatives. In some embodiments, the surfactant is a nonionic surfactant, a surfactant plus nitrogen source, an organo-silicone surfactant, or a high surfactant oil concentrate.

xi. Combinations

In formulations and in the use forms prepared from these formulations, the recombinant polynucleotide can, in embodiments, be in a mixture with other active compounds, such as pesticidal agents (e.g., insecticides, sterilants, acaricides, nematicides, molluscicides, or fungicides, attractants, growth-regulating substances, or herbicides. As used herein, the term “pesticidal agent” refers to any substance or mixture of substances intended for preventing, destroying, repelling, or mitigating any pest. A pesticide can be a chemical substance or biological agent used against pests including insects, mollusks, pathogens, weeds, nematodes, and microbes that compete with humans for food, destroy property, spread disease, or are a nuisance. The term “pesticidal agent” further encompasses other bioactive molecules such as antibiotics, antivirals pesticides, antifungals, antihelminthics, nutrients, pollen, sucrose, and/or agents that stun or slow insect movement.

In instances where the recombinant polynucleotide is applied to plants, a mixture with other known compounds, such as herbicides, fertilizers, growth regulators, safeners, semiochemicals, or else with agents for improving plant properties is also possible.

### II. Effectors

Provided herein are compositions comprising recombinant polynucleotides comprising (i) a single-stranded RNA (ssRNA) viroid sequence and (ii) a heterologous RNA sequence comprising or encoding an effector. The effector can be any moiety that can be integrated into a recombinant polynucleotide comprising an ssRNA viroid sequence (e.g., a recombinant polynucleotide, e.g., a viroid-derived vector) and that has a biological effect on (e.g., is capable of modulating a state of) an organism or a cell thereof, e.g., has a biological effect on a plant or a plant cell; an arthropod or an arthropod cell; a mollusk or a mollusk cell; a fungus or a fungus cell; or a nematode or a nematode cell. In some embodiments, the effector has a biological effect on a plant, e.g., modulates a trait, phenotype, or genotype in a plant, plant part, or plant cell, as described in Section IIIB herein.

In some embodiments, recombinant polynucleotide comprises an RNA sequence (e.g., an ssRNA sequence) that comprises or consists of the effector. In other embodiments, the recombinant polynucleotide comprises an RNA sequence (e.g., an ssRNA sequence) that is modified to produce the effector, e.g., is processed in a cell (e.g., a target cell) to produce the effector. In still other embodiments, the recombinant polynucleotide comprises an RNA sequence (e.g., an ssRNA sequence) that encodes the effector, e.g., an RNA sequence that is translated in a cell (e.g., a target cell) to produce a protein or polypeptide effector. In some embodiments, the RNA sequence is an ssRNA sequence.

In some embodiments, the effector consists of or comprises a coding sequence. In some embodiments, the coding sequence is a protein or a polypeptide.

In some embodiments, the effector consists of or comprises a regulatory RNA, e.g., a long non-coding RNA (lncRNA), a circular RNA (circRNA), a transfer RNA-derived fragment (tRF), a transfer RNA (tRNA), a ribosomal RNA (rRNA), a small nuclear RNA (snRNA), a small nucleolar RNA (snoRNA), or a Piwi-interacting RNA (piRNA).

In some embodiments, the effector consists of or comprises an interfering RNA, e.g., a small RNA (sRNA), a double-stranded RNA (dsRNA); a hairpin RNA (hpRNA), a microRNA (miRNA); a pre-miRNA; a phased, secondary, small interfering RNA (phasiRNA); a heterochromatic small interfering RNA (hcsiRNA); or a natural antisense short interfering RNA (natsiRNA).

In some embodiments, the effector comprises or consists of a hairpin RNA (hpRNA) targeting a transcript of the host cell (e.g., plant cell, arthropod cell, mollusk cell, fungus cell, or nematode cell).

In some embodiments, the effector comprises or consists of a small RNA (sRNA) targeting a transcript of the host cell (e.g., plant cell, arthropod cell, mollusk cell, fungus cell, or nematode cell).

In some embodiments, the effector comprises or consists of a pre-miRNA targeting a transcript of the host cell (e.g., plant cell, arthropod cell, mollusk cell, fungus cell, or nematode cell).

In some embodiments, the effector comprises or consists of a circRNA corresponding to a gene of the host cell (e.g., plant cell, arthropod cell, mollusk cell, fungus cell, or nematode cell).

In some embodiments, the effector comprises or consists of an RNA sequence corresponding to a gene or gene transcript of the host cell (e.g., plant cell, arthropod cell, mollusk cell, fungus cell, or nematode cell).

Exemplary genes that can be targeted by effectors (e.g., regulatory or interfering RNAs) include, e.g., genes encoding hormones, enzymes, and transcription factors.

In some embodiments, the effector consists of or comprises a guide RNA (e.g., a guide RNA for use in combination with a gene editing enzyme). In some embodiments, the effector comprises or consists of a guide RNA (gRNA) targeting a gene of the host cell (e.g., plant cell, arthropod cell, mollusk cell, fungus cell, or nematode cell).

In some embodiments, the effector comprises or consists of an aptamer, e.g., a DNA aptamer, RNA aptamer, or peptide aptamer.

In some aspects, the effector (e.g., RNA, polypeptide, or protein effector) binds a target cell host factor.

In embodiments, the target cell host factor is, e.g., a nucleic acid, a protein, a DNA, or an RNA.

The effector sequence is heterologous with respect to the ssRNA viroid sequence or sequences included in the recombinant polynucleotide. In some embodiments, the RNA sequence comprising or encoding the effector is not a viroid sequence, e.g., is not derived from the sequence of any viroid. In embodiments, the RNA sequence is, e.g., an artificial sequence or a sequence derived from another organism. In other embodiments, the RNA sequence comprising or encoding the effector is derived from a viroid other than the viroid from which the ssRNA viroid sequence of part (i) of the recombinant polynucleotide is derived.

In some embodiments, the recombinant polynucleotide comprises or encodes a single effector. In other embodiments, the recombinant polynucleotide comprises or encodes at least two effectors, e.g., 2, 3, 4, 5, 6, 7, 8, 9, 10, or more than 10 effectors.

### CRISPR Guide RNAs:

In some embodiments, the effector is a CRISPR guide RNA. CRISPR-associated endonucleases such as Cas9, Cas12 and Cas13 endonucleases are used as genome editing tools in different plants; see, e.g., Wolter et al. (2019) BMC Plant Biol., 19:176-183); Aman et al. (2018) Genome Biol., 19:1-10. CRISPR/Cas9 requires a two-component crRNA:tracrRNA “guide RNA” (“gRNA”) that contains a targeting sequence (the “CRISPR RNA” or “crRNA” sequence) and a Cas9 nuclease-recruiting sequence (tracrRNA). Efficient Cas9 gene editing is also achieved with the use of a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in genome editing; see, for example, Hendel et al. (2015) Nature Biotechnol., 985-991. Commercial manufacturers of CRISPR nucleases and guide RNAs provide algorithms for designing guide RNA sequences; see, e.g., guide design tools provided by Integrated DNA Technologies at www[dot]idtdna[dot]com/pages/products/crispr-genome-editing/alt-r-crispr-cas9-system. Some Cas nucleases, including Cas12a and Cas13, do not require a tracrRNA.

For many Cas nucleases, guide sequence designs are constrained by the requirement that the DNA target sequence (to which the crRNA is designed to be complementary) must be adjacent to a proto-spacer adjacent motif (“PAM”) sequence that is recognized by the specific Cas nuclease to be employed. Cas nucleases recognize specific PAM sequences and there is a diversity of nucleases and corresponding PAM sequences; see, e.g., Smakov et al. (2017) Nature Reviews Microbiol., doi:10.1038/nrmicro.2016.184. For example, Cas9 nucleases cleave dsDNA, require a GC-rich PAM sequence located 3′ to the DNA target sequence to be targeted by the crRNA component of the guide RNA, and cleave leaving blunt ends. Cas12a nucleases cleave dsDNA, require a T-rich PAM sequence located 5′ to the DNA target sequence to be targeted by the crRNA component of the guide RNA, and cleave leaving staggered ends with a 5′ overhang. Cas13 nucleases cleave single-stranded RNAs and do not require a PAM sequence; instead, Cas13 nuclease are guided to their targets by a single crRNA with a direct repeat (“DR”). In practice, the crRNA component of a guide RNA is generally designed to have a length of between 17-24 nucleotides (frequently 19, 20, or 21 nucleotides) and exact complementarity (i. e., perfect base-pairing) to the targeted gene or nucleic acid sequence that is itself adjacent to a PAM motif (when required by the Cas nuclease). A crRNA component having less than 100% complementarity to the target sequence can be used (e. g., a crRNA with a length of 20 nucleotides and between 1-4 mismatches to the target sequence) but this increases the potential for off-target effects.

Non-limiting examples of effective guide design are found, e.g., in US Patent Application Publications US 2019/0032131, 2015/0082478, and 2019/0352655, which are incorporated by reference in their entirety herein. For the purposes of gene editing, CRISPR “arrays” can be designed to include one or multiple guide RNA sequences corresponding to one or more desired target DNA sequence(s); see, for example, Cong et al. (2013) Science, 339:819-823; Ran et al. (2013) Nature Protocols, 8:2281-2308.

In an embodiment, an effector moiety integrated into a viroid-derived vector or polynucleotide includes at least one CRISPR guide RNA; release of the guide RNA is mediated, e.g., by flanking DR sequences, ribozyme sequences, or other self-cleaving RNAs, or by cleavage by an endogenous ribonuclease. The corresponding Cas nuclease can be provided by separate or concurrent delivery, e.g., by co-delivery with the viroid-derived vector or polynucleotide, or by transient or stable expression of the corresponding Cas nuclease in the cell to which the viroid-derived vector or polynucleotide is delivered.

siRNAs and Ta-siRNAs

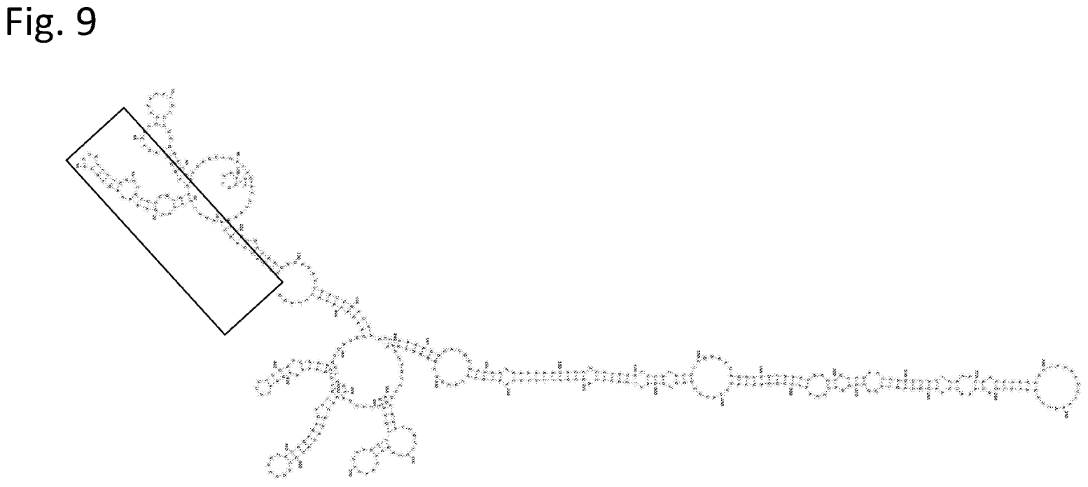

In some embodiments, the effector is a siRNA or a ta-siRNA. A double-stranded RNA (e.g., a dsRNA made of two separate hybridizing RNA strands, or a single RNA strand that forms a stem-loop structure) that includes complementary or hybridizing “sense” and “anti-sense” RNA segments that correspond to (i.e., are respectively identical to, or complementary to, a target gene) can be processed by DICER into asymmetric hybridized pairs of small interfering RNAs (siRNAs) of usually 20-24 base pairs, most often 21-23 base pairs, with 2-nucleotide 3′ overhangs. A hybridized pair of siRNAs is complexed with multiple proteins to form the RNA-induced silencing complex (“RISC”); one strand is preferably bound to the protein Argonaute and acts as a “guide” for the RISC complex in binding to and directing cleavage of a target transcript. In embodiments, the resulting siRNAs silence or decrease the expression of the target gene. In some embodiments, the siRNAs silence transposable elements in heterochromatin. The target gene can be a (protein-) coding or non-coding nucleotide sequence or a combination of coding and non-coding sequence, and can be endogenous to the cell to which the siRNAs are provided, or can be exogenous (e.g., a viral sequence). Various arrangements and combinations of sense and/or anti-sense RNA segments (such as those resulting from transcription of the DNA molecules depicted in FIG. 9 of U.S. Pat. No. 9,708,620, incorporated by reference in its entirety herein) can be provided in a single contiguous polynucleotide, or in multiple, non-contiguous polynucleotide segments, or in multiple polynucleotides, which are processed into siRNAs for silencing or decreasing expression of a given target gene. Selection of sequences for siRNA production is known in the art; see, for example, Wang & Mu (2004), Bioinformatics, 20:1818-1820; Reynolds et al. (2004), Nature Biotechnol., 22:326-330; and Yuan et al. (2004), Nucleic Acids Res., 32:W130-W134. Mismatches between a given siRNA guide sequence and a target transcript can be tolerated, particularly where multiple different siRNAs generated from longer dsRNA stems provide improved overall efficacy, and therefore siRNAs are designed to have at least about 70% complementarity to a segment of equivalent length in the target transcript. However, generally it is most convenient and simplest to design an siRNA that has exact complementarity to the target gene sequence.

In an embodiment, an effector moiety integrated into a viroid-derived vector or polynucleotide includes at least one double-stranded RNA stem designed to be processed into siRNAs for RNAi-mediated silencing or decrease of expression of a target gene. In other embodiments, the viroid-derived vector or polynucleotide includes at least one effector moiety that includes one or more double-stranded RNA stems designed to be processed into siRNAs for RNAi-mediated silencing or decrease of one or more target genes. The length of a double-stranded RNA stem is selected for efficacy and convenience. For efficacy, the double-stranded RNA stem includes at least 19 contiguous base pairs, and where the viroid-derived vector or polynucleotide is a circular RNA, the double-stranded RNA stem preferably includes at least 23 base pairs, e.g., 23, 24, 25, 26, 27, 28, 29, 30, 23-30, 23-40, 30-40, 30-50, 40-60, 40-80, or 50-100 base pairs; see, e.g., Abe et al. (2007) J. Am. Chem. Soc., 129:15108-15109. The double-stranded RNA stem can be longer, in the order of a few hundred base pairs, e.g., 80-150, 100-200, 150-250, or 200-300 base pairs. For convenience and economy of production and in consideration of steric effects, generally the overall length of a given double-stranded RNA stem is no longer than necessary to obtain the desired level of silencing or decrease in expression of the target gene(s).

Alternatively, multiple siRNAs can be produced from a single-stranded RNA transcript designed to generate multiple “trans-acting siRNAs” (ta-siRNAs). Production ofta-siRNAs is initiated by cleavage of the ssRNA transcript at a 5′-proximal site, followed by amplification of the 3′ RNA product by RNA-dependent RNA polymerase 6 (RDR6) and processing by a specific Dicer-type enzyme, DCL4, to yield phased siRNAs (the “ta-siRNAs”) that are produced in 21-nucleotide register with the cleavage site and therefore can be designed to target specific genes. See, e.g., U.S. Pat. Nos. 8,030,473, 8,476,422, 8,816,061, and 9,018,002, which are incorporated by reference in their entirety herein. Also see, e.g., Allen et al. (2005) Cell, 121:207-221 and Cuperus et al. (2010) Nature Structural Mol. Biol., 17:997-1003.

miRNAs, Phased Small RNAs:

In an embodiment, an effector integrated into a viroid-derived vector or polynucleotide includes at least one microRNA (miRNA) precursor, which can have the sequence of a naturally occurring miRNA precursor that is processed into a naturally occurring mature miRNA, or of an artificial (synthetic) miRNA precursor sequence that is processed into an artificial mature miRNA. Mature miRNAs are small RNAs, typically of 19, 20, 21, 22, 23, 24, 25, or 26 nucleotides in length; a mature miRNA binds to complementary sequences (“miRNA recognition site”) in gene transcripts, leading to cleavage and silencing or decreasing expression of that gene. In nature, a mature miRNA is encoded in a miRNA precursor, a single-stranded RNA transcript that forms a fold-back structure typically including an imperfect stem-loop containing mismatches and “bulges”, at least some of which occur between the nucleotides that make up the mature miRNA and the nucleotides that make up the so-called “mir*” (“mir-star”) sequence located on the opposite strand of the precursor's stem. Upon normal processing within the cell, the mature miRNA is released. Numerous naturally occurring miRNAs and their corresponding miRNA precursors have been described and can be accessed on a public, searchable database, miRbase (available on line at www[dot]miRbase[dot]org); see, e.g., Kozomara et a. (2019) Nucleic Acids Res., 47:D155-D162; Griffiths-Jones et al. (2008) Nucleic Acids Res., 36:D154-D158; Griffiths-Jones et al. (2004) Nucleic Acids Res., 32:D109-D111.

Artificial mature miRNAs for “silencing” a selected target gene are designed to have a sequence that is complementary to the target gene's transcript, thus allowing the artificial mature miRNA to specifically bind to and cleave the RNA transcript of that target gene. In an embodiment, an engineered miRNA precursor molecule is designed using as a design scaffold or template a native miRNA precursor sequence that encodes a native mature miRNA, with nucleotides of the native mature miRNA replaced with nucleotides that are complementary to the target RNA transcript, while maintaining the position and number of mismatches in the stem portion of the miRNA precursor's fold-back structure by altering as needed the miR* nucleotides in the precursor strand, and generally leaving the remaining nucleotides of the miRNA precursor unchanged. Non-limiting examples illustrating the design of such artificial miRNAs based on naturally occurring plant or invertebrate miRNA precursors are described respectively in, e.g., U.S. Pat. Nos. 7,786,350, 8,395,023, 8,946,511, and 9,708,620, and 8,410,334 and 10,570,414, which are incorporated by reference in their entirety herein.

One general, non-limiting method for selecting a nucleotide sequence for an artificial mature miRNA (therefore determining nucleotide changes in the native miRNA precursor sequence to produce the artificial miRNA precursor) includes these steps:

Selecting a unique target sequence of at least 18 nucleotides, preferably at least 19 nucleotides, that is specific to the target gene, thereby avoiding unintentional silencing of non-target sequences, e.g., by using sequence alignment tools such as BLAST (see, for example, Altschul et al. (1990) J. Mol. Biol., 215:403-410; Altschul et al. (1997) Nucleic Acids Res., 25:3389-3402), to identify from genomic sequence any target transcript orthologues and any potential matches to unrelated genes that should be avoided; Scoring each potential 19-mer segment along the length of the target gene for GC content, Reynolds score (see Reynolds et al. (2004) Nature Biotechnol., 22:326-330), and functional asymmetry characterized by a negative difference in free energy (“ΔΔG”) (see Khvorova et al. (2003) Cell, 115:209-216); preferably 19-mers are selected that have all or most of the following characteristics: (1) a Reynolds score >4, (2) a GC content between about 40% to about 60%, (3) a negative “ΔΔG”, (4) a terminal adenosine, (5) lack of a consecutive run of 4 or more of the same nucleotide; (6) a location near the 3′ terminus of the target gene; (7) minimal differences from the miRNA precursor transcript; Determining the reverse complement of the selected 19-mers to use in making a modified mature miRNA; the additional nucleotide at position 20 is preferably matched to the selected target sequence, and the nucleotide at position 21 is preferably chosen to be unpaired to prevent spreading of silencing on the target transcript;

Optionally, testing the engineered miRNA precursor, for example, in an Agrobacterium-mediated transient Nicotiana benthamiana assay, for efficacy. Multiple 19-mers can be selected for testing, in which case the most effective engineered miRNA precursor sequence(s) can be selected for further use.

Similarly, phased small RNAs such as those described in U.S. Pat. Nos. 8,404,928 and 9,309,512, which are incorporated by reference in their entirety herein, can be engineered to bind and cleave one or multiple selected RNA transcripts. The phased small RNA precursor, which contains multiple ˜21-mer small RNAs, forms an extended imperfect stem-loop containing mismatches and bulges. Any one or more of the contiguous 21-mers found within this precursor can be designed to bind to and cleave a target RNA. Phased small RNAs can be designed in a manner similar to that used for designing an artificial miRNA using the criteria for selecting a nucleotide sequence for an artificial mature miRNA described above. The artificial phased small RNA precursor can be tested and the most effective phased small RNAs can be selected for further use.

### III. Methods of Use

### A. Methods of Delivery to Plants

In some aspects, the disclosure features a method of delivering an effector to a plant, a plant tissue, or a plant cell, the method comprising providing to the plant, plant tissue, or plant cell a composition described herein (e.g., a composition comprising or consisting of a recombinant polynucleotide (e.g., a vector) comprising (i) a single-stranded RNA (ssRNA) viroid sequence and (ii) a heterologous RNA sequence comprising or encoding an effector, whereby the effector comprised by or encoded by the heterologous RNA sequence is delivered to the plant, plant tissue, or plant cell. In embodiments, the effector is, e.g., any effector described in Section II herein. In some embodiments, providing comprises contacting the plant with the recombinant polynucleotide.

In some aspects, the disclosure features a plant, plant tissue, or plant cell comprising a recombinant polynucleotide of the disclosure (e.g., an ssRNA recombinant polynucleotide (e.g. a circular ssRNA) or a DNA molecule encoding such a polynucleotide).

i. Methods of delivery

A plant described herein can be exposed to any of the compositions described herein in any suitable manner that permits delivering or administering the composition to the plant. The modulating agent can be delivered either alone or in combination with other active or inactive substances and can be applied by, for example, spraying, leaf rubbing, microinjection, in a hydroponic system (e.g., to roots), pouring, dipping, in the form of concentrated liquids, gels, solutions, suspensions, sprays, powders, pellets, briquettes, bricks and the like, formulated to deliver an effective concentration of the recombinant polynucleotide. Amounts and locations for application of the compositions described herein are generally determined by the anatomy and physiology of the plant, the lifecycle stage at which the recombinant polynucleotide is to be delivered, the site where the application is to be made, and the physical and functional characteristics of the recombinant polynucleotide.

In some instances, the composition is sprayed directly onto a plant, e.g., by backpack spraying, aerial spraying, crop spraying/dusting, etc.. The plant receiving the recombinant polynucleotide can be at any stage of plant growth. For example, recombinant polynucleotides can be applied as a seed-coating or root treatment in early stages of plant growth or as a total plant treatment at later stages of the crop cycle. Further, the recombinant polynucleotide can be applied as a systemic agent (e.g., in the soil in which a plant grows, in the water that is used to water the plant, or in a hydroponic system in which the plant is grown or cultured) that is absorbed and distributed through the tissues (e.g., roots, leaves, and/or stems) of a plant. In some instances, plants are genetically transformed to express the recombinant polynucleotide. In some embodiments, systemic or transgenic applications are used such that an insect, mollusk, nematode, or fungus feeding on the plant will obtain an effective dose of the recombinant polynucleotide and/or the effector.

Delayed or continuous release can be accomplished by coating the recombinant polynucleotide or a composition containing the recombinant polynucleotide with a dissolvable or bioerodable coating layer, such as gelatin, which coating dissolves or erodes in the environment of use, to then make the recombinant polynucleotide available, or by dispersing the agent in a dissolvable or erodable matrix. Such continuous release and/or dispensing means devices can be advantageously employed to consistently maintain an effective concentration of one or more of the recombinant polynucleotides described herein in a specific host habitat.

In some embodiments, providing the composition to the plant, plant tissue (e.g., dermal, ground (e.g., leaf, stem, and root), or vascular tissue (e.g., xylem and phloem)), or plant cell comprises delivering the composition to a leaf, root, stem, flower, seed, xylem, phloem, apoplast, symplast, meristem, fruit, embryo, microspore, pollen, pollen tube, ovary, ovule, or explant for transformation of the plant. In some embodiments, the plant is a monocot or a dicot. In some embodiments, the plant cell is a protoplast. In some embodiments, the fruit is pre-harvest fruit. In other embodiments, the fruit is a post-harvest fruit.

In some embodiments, the recombinant polynucleotide is delivered to the plant using a method described in U.S. patent Ser. No. 10/597,676, 10655136, 9121022, 10378012, or 8367895 or PCT publication WO2018140899 or WO2018085693.

### Delivery to the Nucleus of a Plant Cell

In some aspects, the disclosure provides a method of delivering an RNA effector to the nucleus of a plant cell, comprising contacting a plant cell with a synthetic nuclear transporter comprising: (i) a single-stranded RNA (ssRNA) viroid sequence and (ii) a heterologous RNA sequence comprising or encoding an effector, wherein the ssRNA viroid sequence does not include a chloroplast localization sequence; wherein the synthetic nuclear transporter localizes to the nucleus of the plant cell, thereby delivering the effector to the nucleus.

In some aspects, the ssRNA viroid sequence has at least 80% sequence identity with a pospiviroid sequence.

In some aspects, the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467.

In some aspects, the ssRNA viroid sequence has at least 90% sequence identity with SEQ ID NO:51.

In some aspects, the heterologous RNA sequence comprises coding RNA, non-coding RNA, or both coding and non-coding RNA.

In some aspects, the effector comprises coding RNA, non-coding RNA, or both coding and non-coding RNA.

In some aspects, the effector comprises non-coding RNA comprising at least one regulatory RNA or at least one interfering RNA that targets a transcript in a cell. In some aspects, the cell is selected from the group consisting of a plant cell, an arthropod cell, a mollusk cell, a fungus cell, or a nematode cell.

In some aspects, the disclosure provides a composition comprising a synthetic nuclear transporter, wherein the synthetic nuclear transporter comprises: ( ) a single-stranded RNA (ssRNA) viroid sequence and (ii) a heterologous RNA sequence comprising or encoding an effector, wherein the ssRNA viroid sequence does not include a chloroplast localization sequence.

In some aspects, the ssRNA viroid sequence has at least 80% sequence identity with a pospiviroid sequence.

In some aspects, the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467.

In some aspects, the ssRNA viroid sequence has at least 90% sequence identity with SEQ ID NO:51.

In some aspects, the heterologous RNA sequence comprises coding RNA, non-coding RNA, or both coding and non-coding RNA.

In some aspects, the effector comprises coding RNA, non-coding RNA, or both coding and non-coding RNA.

iii. Replication and Transmission by Inheritance

In some embodiments, the recombinant polynucleotide (e.g., RNA) replicates within the plant.

In some embodiments, the cell is transiently transformed with the recombinant polynucleotide. In other embodiments, the cell is stably transformed with the recombinant polynucleotide, e.g., the recombinant polynucleotide or a portion thereof (e.g., a portion comprising the effector) is integrated into the nuclear genome, chloroplast genome, or mitochondrial genome.

In some embodiments, the recombinant polynucleotide is inherited by a progeny of the plant (e.g., a seed of the plant, a seed fertilized by pollen of the plant, or an asexually propagated clone of the plant (e.g., a plantlet, cutting, runner, bulb, tuber, corm, sucker, or tissue culture of the plant)). In other embodiments, the recombinant polynucleotide is not inherited by a progeny of the plant, e.g., is not transmitted in pollen and/or seeds.

iii. Plants, Plant Parts, and Plant Cells

Plants, plant parts, and plant cells are of any species of interest, including flowering plants (e.g., dicots and monocots); gymnosperms; seedless vascular plants (e.g., fems); bryophytes (e.g., mosses); algae; and cyanobacteria. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sadiva), almonds (Prunus dulcis), apples (Malus x domestica), apricots (Prunus armeniaca, P. brigantine, P. mandshurica, P. mume, P. sibirica), asparagus (Asparagus officinalis), bananas (Musa spp.), barley (Hordeum vulgare), beans (Phaseolus spp.), blueberries and cranberries (Vaccinium spp.), cacao (Theobroma cacao), canola and rapeseed or oilseed rape, (Brassica napus), Polish canola (Brassica rapa), and related cruciferous vegetables including broccoli, kale, cabbage, and tumips (Brassica carnata, B. juncea, B. oleracea, B. napus, B. niga, and B. rapa, and hybrids of these), carnation (Dianthus caryophyllus), carrots (Daucus carota sativus), cassava (Manihot esculentum), cherry (Prunus avium), chickpea (Cicer arietinum), chicory (Cichorium intybus), chili peppers and other capsicum peppers (Capsicum annuum, C. frutescens, C. chinense, C. pubescens, C. baccatum), chrysanthemums (Chrysanthemum spp.), coconut (Cocos nucifiera), coffee (Coffea spp. including Coffea arabica and Coffea canephora), cotton (Gossypium hirsutum L.), cowpea (Vigna unguiculata and other Vigna spp.), fava bean (Vicia faba), cucumber (Cucumis sativus), currants and gooseberries (Ribes spp.), date (Phoenix dactylifera), duckweeds (family Lemnoideae), eggplant or aubergine (Solanum melongena), eucalyptus (Eucalyptus spp.), flax (Linum usitadissumum L.), geraniums (Pelargonium spp.), grapefruit (Citrus x paradis), grapes (Vitus spp.) including wine grapes (Vitus vinifera and hybrids thereof), guava (Psidium guajava), hops (Humulus lupulus), hemp and cannabis (Cannabis sativa and Cannabis spp.), irises (Iris spp.), lemon (Citrus limon), lettuce (Lactuca salva), limes (Citrus spp.), maize (Zea mays L.), mango (Mangifera indica), mangosteen (Garcinia mangostana), melon (Cucumis melo), millets (Setaria spp., Echinochloa spp., Eleusine spp., Panicum spp., Pennisetum spp.), oats (Avena sativa), oil palm (Ellis quineensis), olive (Olea europaea), onion (Allium cepa) and other alliums (Allium spp.), orange (Citrus sinensis), papaya (Carica papaya), peaches and nectarines (Prunus persica), pear (Pyrus spp.), pea (Pisa sativum), peanut (Arachis hypogaea), peonies (Paeonia spp.), petunias (Petunia spp.), pineapple (Ananas comosus), plantains (Musa spp.), plum (Prunus domestica), poinsettia (Euphorbia pulcherrima), poplar (Populus spp.), potato (Solanum tuberosum), pumpkins and squashes (Cucurbita pepo, C. maximus, C. moschata), rice (Oryza sativa L.), roses (Rosa spp.), rubber (Hevea brasiliensis), rye (Secale cereale), safflower (Carthamus tinctorius L), sesame seed (Sesame indium), sorghum (Sorghum bicolor), soybean (Glycine max L.), strawberries (Fragara spp., Fragaria x ananassa), sugar beet (Beta vulgaris), sugarcanes (Saccharum spp.), sunflower (Helianthus annuus), sweet potato (Ipomoea batatas), tangerine (Citrus tangerina), tea (Camellia sinensis), tobacco (Nicotiana tabacum L.), tomato (Solanum lycopersicum or Lycopersicon esculentum), tulips (Tulipa spp.), walnuts (Juglans spp. L.), watermelon (Citrulus lanatus), wheat (Triticum aestivum), and yarns (Discorea spp.).

The plant cells and derivative plants and seeds disclosed herein can be used for various purposes useful to the consumer or grower. In embodiments, the intact plant itself is desirable, e.g., plants grown as cover crops or as ornamentals. In other embodiments, processed products are made from the plant or its seeds, such as extracted proteins, oils, sugars, and starches, fermentation products, animal feed or human food, wood and wood products, pharmaceuticals, and various industrial products. Thus, further related aspects of the disclosure include a processed or commodity product made from a plant or seed or plant part that includes at least some cells that contain the recombinant polynucleotide or the effector. Commodity products include, but are not limited to, harvested leaves, roots, shoots, tubers, stems, fruits, seeds, or other parts of a plant, meals, oils (edible or inedible), fiber, extracts, fermentation or digestion products, crushed or whole grains or seeds of a plant, wood and wood pulp, or any food or non-food product.

In some aspects, the plant is a weed. As used herein, the term “weed” refers to a plant that grows where it is not wanted. Such plants are typically invasive and, at times, harmful, or have the risk of becoming so. In embodiments, weeds are treated with the present pest control (e.g., biopesticide or biorepellent) compositions to reduce or eliminate the presence, viability, or reproduction of the plant. For example, and without being limited thereto, the methods can be used to target weeds known to damage plants. For example, and without being limited thereto, the weeds can be any member of the following group of families: Gramineae, Umbelliferae, Papilionaceae, Cruciferae, Malvaceae, Eufhorbiaceae, Compositae, Chenopodiaceae, Fumariaceae, Charyophyllaceae, Primulaceae, Geraniaceae, Polygonaceae, Juncaceae, Cyperaceae, Aizoaceae, Asteraceae, Convolvulaceae, Cucurbitaceae, Euphorbiaceae, Polygonaceae, Portulaceae, Solanaceae, Rosaceae, Simaroubaceae, Lardizabalaceae, Liliaceae, Amaranthaceae, Vitaceae, Fabaceae, Primulaceae, Apocynaceae, Araliaceae, Caryophyllaceae, Asclepiadaceae, Celastraceae, Papaveraceae, Onagraceae, Ranunculaceae, Lamiaceae, Commelinaceae, Scrophulariaceae, Dipsacaceae, Boraginaceae, Equisetaceae, Geraniaceae, Rubiaceae, Cannabaceae, Hyperiacaceae, Balsaminaceae, Lobeliaceae, Caprifoliaceae, Nyctaginaceae, Oxalidaceae, Vitaceae, Urticaceae, Polypodiaceae, Anacardiaceae, Smilacaceae, Araceae, Campanulaceae, Typhaceae, Valerianaceae, Verbenaceae, Violaceae. For example, and without being limited thereto, the weeds can be any member of the group consisting of Lolium rigidum, Amaramthus palmeri, Abublon Iheopratsi, Sorghum halepense, Conyza canadensis, Setaria verticillata, Capsella pastoris, and Cyperus rotundas. Additional weeds include, for example, Mimosa pigra, Salvinia spp., Hypti spp., Senna spp., noogoora burr (Xanthium spinosum) and other burr weeds, Jatropha gossypilblia, Parkinsonia aculeate, Chromolaena odorata, Cryptoslegla grandilora, and Andropogon gayanus. Weeds can include monocotyledonous plants (e.g., Agrostis, Alopecurus, Avena, Bromus, Cyperus, Digitaria, Echinochloa, Lolium, Monochoria, Rottboellia, Sagittaria, Scirpus, Setaria, Sida, or Sorghum) or dicotyledonous plants (Abutilon, Amaranthus, Chenopodium, Chrysanthemum, Conyza, Galium, Ipomoea, Nasturtium, Sinapis, Solanum, Stellaria, Veronica, Viola, or Xanthium).

In some embodiments, a composition comprising a recombinant polynucleotide comprising (i) a single-stranded RNA (ssRNA) viroid sequence and (ii) a heterologous RNA sequence comprising or encoding an effector is delivered to a plant that the viroid is known to infect, e.g., a plant in which the viroid has been observed. In other embodiments, a composition comprising a recombinant polynucleotide comprising (i) a single-stranded RNA (ssRNA) viroid sequence and (ii) a heterologous RNA sequence comprising or encoding an effector is delivered to a plant that the viroid has not been observed to infect. Descriptions of viroids infecting a range of host species (including horticultural and crop species) are provided, e.g., in Bagherian et al., Journal of Plant Physiology, 201: 42-53, 2016; Singh et al., Virus Dis., 25(4): 415-424, 2014; and Constable et al., Viruses, 11(98): 2019.

In some embodiments, a composition comprising a recombinant polynucleotide comprising (i) a single-stranded RNA (ssRNA) viroid sequence and (ii) a heterologous RNA sequence comprising or encoding an effector is delivered to any plant, plant part, or plant cell type. A plant can be, e.g., an entire plant, e.g., an entire adult plant, juvenile plant, seedling, or embryo of any of the plant species described herein. Plant parts include, but are not limited to leaves (e.g., leaf blade, leaflet, phyllode, or petiole), seeds (including embryo, endosperm, or seed coat), roots (e.g., primary roots, secondary roots, radicles, root hairs, or root nodules), shoot vegetative organs/structures (e.g., leaves, stems, hypocotyls, rhizomes, or tubers), flowers and floral organs/structures (e.g., pollen, bracts, sepals, petals, stamens, carpels, anthers, or ovules), fruits (including mature ovaries and associated tissues, e.g., receptacle, hypanthium, or perianth), vegetables, pollen, seeds, spores, sap (e.g., phloem or xylem sap), or plant tissues (e.g., vascular tissue, ground tissue, parenchyma, sclerenchyma, collenchyma, or tumor tissue). Plant cell types include any cells of plants and plant parts described herein (e.g., epidermal cells, mesophyll cells, vasculature cells, parenchymal cells, meristematic cells, and root cells) and protoplasts thereof (e.g., leaf protoplasts or root protoplasts. In some aspects, the plant is a single-celled plant, e.g., a single-celled plastid-containing organism such as algae. In embodiments, the composition is delivered to only part of a plant, such as to meristematic tissue of a plant, or to rootstock onto which an untreated scion is grafted, or to a scion that is grafted onto untreated rootstock.

In some embodiments, a composition comprising a recombinant polynucleotide comprising (i) a single-stranded RNA (ssRNA) viroid sequence and (ii) a heterologous RNA sequence comprising or encoding an effector is delivered to an unorganized cell culture, in which a plurality of the cultured cells are not organized into a tissue or organ of a multicellular plant, such as a leaf, root, shoot, or reproductive structure of a multicellular plant. Exemplary unorganized cell cultures include callus culture, cell suspension culture, and protoplast culture.

In some embodiments, the disclosure features a cell comprising a composition described herein. In some embodiments, the cell is a plant cell, e.g., a monocot cell or a dicot cell. In some embodiments, the plant cell is a protoplast. In some embodiments, the cell has been transiently transformed with the recombinant polynucleotide. In other aspects, the cell has been stably transformed with the recombinant polynucleotide.

### B. Methods of Modifying Plants

In some aspects, the disclosure provides a method of modulating a trait, phenotype, or genotype in a plant, plant part, or plant cell, the method comprising providing to the plant, plant part, or plant cell a composition described herein (e.g., a composition comprising or consisting of a recombinant polynucleotide (e.g., a vector) comprising (i) a single-stranded RNA (ssRNA) viroid sequence and (ii) a heterologous RNA sequence comprising or encoding an effector). The effector can be any moiety that can be integrated into a recombinant polynucleotide comprising an ssRNA viroid sequence (e.g., a viroid-derived vector) and that has a biological effect on (e.g., is capable of modulating a state of) a plant or a plant cell.

In some embodiments, modulating comprises expressing in the plant a protein or polypeptide, wherein the heterologous protein or polypeptide is encoded by the heterologous RNA sequence of the recombinant polynucleotide. The protein or polypeptide can be, e.g., a native protein or polypeptide of the plant to which the composition is delivered; a protein or polypeptide of another organism; or an artificial protein or polypeptide.

In some embodiments, modulating comprises reducing expression of a target gene of the plant. In embodiments, expression of the target gene is reduced by about 1%, 2%, 3%, %, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to a reference level (e.g., a level found in a plant, plant part, or plant cell that does not receive a recombinant polynucleotide of the disclosure).

In some embodiments, modulating comprises increasing expression of a target gene of the plant. In embodiments, expression of the target gene is increased by about 1%, 2%, 3%, %, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100% relative to a reference level (e.g., a level found in a plant, plant part, or plant cell that does not receive a recombinant polynucleotide of the disclosure).

In some embodiments, modulating comprises regulating a target gene in the plant. In embodiments, the regulation is, e.g., regulation of transcription; regulation of RNA processing; regulation of translation; regulation of post-transcriptional modification; regulation of expression; regulation of post-translational modification; or regulation of degradation. In some embodiments, a status of the target gene (e.g., transcription, RNA processing, post-transcriptional modification, translation, post-translational modification, expression, or degradation) is decreased by about 1%, 2%, 3%, %, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to a reference level (e.g., a level found in a plant, plant part, or plant cell that does not receive a recombinant polynucleotide of the disclosure). In other aspects, a status of the target gene is increased by about 1%, 2%, 3%, %, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100% relative to a reference level (e.g., a level found in a plant, plant part, or plant cell that does not receive a recombinant polynucleotide of the disclosure).

In some embodiments, modulating (e.g., modifying) comprises editing a target gene of the plant, e.g., editing gene encoded by the nuclear genome, plastid genome, or mitochondrial genome of the plant. In embodiments, the edited gene is inherited by a progeny of the plant (e.g., a seed of the plant, a seed fertilized by pollen of the plant, or an asexually propagated clone of the plant (e.g., a plantlet, cutting, runner, bulb, tuber, corm, sucker, or tissue culture of the plant)).

In some embodiments, the effector increases the fitness of the plant, e.g., increases the fitness of the plant by about 1%, 2%, 3%, %, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100% relative to a reference level (e.g., a level found in a plant, plant part, or plant cell that does not receive a recombinant polynucleotide of the disclosure).

In some embodiments, the effector decreases the fitness of the plant, e.g., increases the fitness of the plant by about 1%, 2%, 3%, %, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100% relative to a reference level (e.g., a level found in a plant, plant part, or plant cell that does not receive a recombinant polynucleotide of the disclosure).

Traits, phenotypes, and genotypes that can be modulated by a composition of the disclosure include, but are not limited to traits, phenotypes, and genotypes that increase or decrease plant fitness. In some embodiments of the compositions described herein, the increase in fitness is an increase in developmental rate, growth rate, size, yield (e.g., intrinsic yield), resistance to abiotic stressors, or resistance to biotic stressors relative to a reference level (e.g., a level found in a plant, plant part, or plant cell that does not receive a recombinant polynucleotide of the disclosure). In some embodiments, the increase in plant fitness is an increase in disease resistance, drought tolerance, heat tolerance, cold tolerance, salt tolerance, metal tolerance, herbicide tolerance, herbicide resistance, chemical tolerance, environmental stress resistance, water use efficiency, nitrogen utilization, resistance or tolerance to nitrogen stress (e.g., low or high nitrogen supply), nitrogen fixation, pest resistance, herbivore resistance, pathogen resistance, disease resistance, fungal disease resistance, virus resistance, nematode resistance, bacterial disease resistance, insect control, yield underwater-limited conditions, vigor, photosynthetic capability, nutrition (e.g., human or animal nutrition), flavor, starch production, protein content, carbohydrate content, oil content, fatty acid content, lipid content, digestibility, biomass, shoot length, root length, root architecture, seed set, seed weight, seed quality (e.g., nutritional content), germination, fruit set, rate of fruit ripening, production of biopolymers, production of fibers, production of biofuels, production of pharmaceutical peptides, production of secretable peptides, enzyme production, improved processing traits, or amount of harvestable produce relative to a reference level (e.g., a level found in a plant, plant part, or plant cell that does not receive a recombinant polynucleotide of the disclosure). In some embodiments, the increase in fitness is earlier flowering. In some embodiments, the increase in plant fitness is an increase in the quality of products harvested from the plant. In some embodiments, the increase in plant fitness is an improvement in taste, appearance, or shelf-life of a product harvested from the plant relative to a reference level (e.g., a level found in a plant, plant part, or plant cell that does not receive a recombinant polynucleotide of the disclosure). In some embodiments, the increase in fitness is a decrease in production of an allergen that stimulates an immune response in an animal. In some embodiments, the trait, phenotype, or genotype that is modulated by a composition of the disclosure is of horticultural interest, e.g., relates to flower size, flower color, flower patterning, flower morphology, flower number, flower longevity, flower fragrance, leaf size, leaf color, leaf patterning, leaf morphology, plant height, or plan architecture.

Genotypes that can be modulated by a composition of the disclosure include genes of agronomic interest. As used herein, the term “gene of agronomic interest” refers to a transcribable polynucleotide molecule that, when expressed in a particular plant tissue, cell, or cell type, confers a desirable characteristic, e.g., a characteristic associated with plant morphology, physiology, growth, development, yield, product, nutritional profile, disease or pest resistance, and/or environmental or chemical tolerance. Genes of agronomic interest include, but are not limited to, those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. In embodiments, the product of a gene of agronomic interest acts within the plant in order to cause an effect upon the plant physiology or metabolism or acts as a pesticidal agent in the diet of a pest that feeds on the plant. Exemplary genes of interest include those described in U.S. patent Ser. No. 10/550,401.

### C. Methods of Use in Eukaryotes

In some aspects, the disclosure features a method of delivering an effector to a eukaryote, comprising providing to the eukaryote a composition comprising a recombinant polynucleotide comprising: (i) a single-stranded RNA (ssRNA) viroid sequence and (ii) a heterologous RNA sequence comprising or encoding an effector, whereby the effector comprised by or encoded by the heterologous RNA sequence is delivered to the eukaryote. In some embodiments, the eukaryote is a plant, a fungus, or an animal.

In some embodiments, the composition is provided to a plant, plant tissue, or plant cell, or a processed product thereof, wherein the eukaryote consumes or contacts the plant, plant tissue, or plant cell, or processed product thereof, whereby the effector is delivered to the eukaryote.

In some embodiments, (a) the ssRNA viroid sequence is a viroid genome or a derivative thereof or (b) the ssRNA viroid sequence is a viroid genome fragment or a derivative thereof.

In some embodiments, the ssRNA viroid sequence is a sequence of a viroid from the family Pospiviroidae or Avsunviroidae. In some embodiments, the viroid is potato spindle tuber viroid (PSTVd) or eggplant latent viroid (ELVd).

In some embodiments, the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467. In some embodiments, the ssRNA viroid sequence has at least 90% sequence identity to SEQ ID NO:51 or SEQ ID NO:50.

In some embodiments, the RNA sequence comprising or encoding the effector is not a viroid sequence and (a) has a biological effect on a plant or (b) has a biological effect on an animal or fungus that consumes or contacts the plant.

In some embodiments, the effector comprises non-coding RNA comprising at least one regulatory RNA or at least one interfering RNA that regulates a target gene or its transcript in a target cell. In some embodiments, the target cell is selected from the group consisting of a plant cell, an animal cell, and a fungal cell.

In some embodiments, the effector modifies a trait, phenotype, or genotype in the target cell. In some embodiments, modifying comprises reducing expression of the target gene. In some embodiments, modifying comprises increasing expression of the target gene. In some embodiments, modifying comprises (a) editing the target gene or (b) regulating the target gene.

In some embodiments, the ssRNA viroid sequence effects one or more results selected from the group consisting of entry into a tissue or cell of the eukaryote; transmission through a tissue or cell or subcellular component of the eukaryote; replication in a tissue or cell of the eukaryote; targeting to a tissue or cell of the eukaryote; and binding to a factor in a tissue or cell of the eukaryote.

In some embodiments, the recombinant polynucleotide lacks free ends and/or is circular.

In some embodiments, the composition is topically delivered to a plant. In some embodiments, the topical delivery is spraying, leaf rubbing, soaking, coating, injecting, seed coating, or delivery through root uptake.

In another aspect, disclosed herein is a composition comprising a recombinant polynucleotide comprising: (a) a single-stranded RNA (ssRNA) viroid sequence that is a viroid genome or a derivative thereof or a viroid genome fragment or a derivative thereof, and (b) a heterologous RNA sequence that is not a viroid sequence and comprises or encodes an effector.

In some embodiments, the viroid genome is (a) a genome of a viroid from the family Pospiviroidae or Avsunviroidae, or (b) a genome of potato spindle tuber viroid (PSTVd) or eggplant latent viroid (ELVd).

In some embodiments, the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467. In some embodiments, the ssRNA viroid sequence has at least 90% sequence identity to SEQ ID NO:51 or SEQ ID NO:50.

In some embodiments, the effector comprises non-coding RNA comprising at least one regulatory RNA or at least one interfering RNA or at least one guide RNA that regulates or modifies a target gene or its transcript in a target cell, wherein the target cell is a plant cell, an animal cell, or a fungal cell.

In some embodiments, the effector (a) modifies expression of a target gene in a eukaryotic cell; or (b) has a biological effect on a plant or on an animal or fungus that consumes or contacts the plant.

In some embodiments, the composition is (a) formulated for delivery to a plant or to the environment in which the plant grows; or (b) formulated for delivery to an animal or fungus.

In embodiments, the eukaryote is a eukaryotic cell, a eukaryotic tissue, a eukaryotic organ, or a eukaryotic organism at any developmental stage. In embodiments, the eukaryote is a plant or a plant seed. In other embodiments, the eukaryote is an animal, a eukaryotic alga, or a fungus. In embodiments, the eukaryote is a vertebrate animal (e.g., mammal, bird, cartilaginous or bony fish, reptile, or amphibian).

In embodiments, the eukaryote is a human; including adults and non-adults (infants and children). In embodiments, the eukaryote is a non-human mammal, such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the eukaryote is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the eukaryote is an invertebrate such as an arthropod (e.g, insects, arachnids, crustaceans), a nematode, an annelid, a helminth, or a mollusc. In embodiments, the eukaryote is an invertebrate agricultural pest, or an invertebrate that is parasitic on an invertebrate or vertebrate host. In embodiments, the eukaryote is a fungus, such as a fungal pathogen of plants, invertebrates, or vertebrates, or a beneficial fungus. In embodiments, the eukaryote is an organism that is part of a symbiosis (e.g., a beneficial fungus that colonizes plant roots or is part of the root/soil-associated microbiome). In embodiments, the eukaryote is a plant, such as an angiosperm plant (which can be a dicot or a monocot) or a gymnosperm plant (e.g., a conifer, a cycad, a gnetophyte, a Ginkgo), a fem, horsetail, clubmoss, or a bryophyte. In embodiments, the eukaryote is a eukaryotic alga (unicellular or multicellular). In embodiments, the eukaryote is a plant of agricultural or horticultural importance, such as row crop plants, fruit-producing plants and trees, vegetables, trees, and ornamental plants including ornamental flowers, shrubs, trees, groundcovers, and turf grasses. Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses.

In an embodiment, the composition is delivered to a plant or plant tissue or plant cell (e.g., by topical spraying or dusting, or injection into the plant's vascular system, or by root soaking or drenching, or by coating a seed), or to the environment in which a plant grows (e.g., as granules or powders or liquids applied to the soil or other growing medium in which a plant grows, or as an additive to a hydroponic system) and the eukaryote consumes or contacts the plant, plant tissue, or plant cell, or a processed product made from the plant, plant tissue, or plant cell, whereby the effector is delivered to the eukaryote.

In an example, the composition is topically sprayed on a plant, and the effector is delivered to an invertebrate that feeds on the plant. In another example, the composition is coated onto a seed, and the effector is delivered to an invertebrate pest that feeds on the seed or the plant that germinates from the seed, or to a fungus that contacts the seed or the plant that germinates from the seed. In another example, the composition is delivered to a plant, plant tissue, or plant cell (which can be a plant cell culture), and a vertebrate or invertebrate animal consumes or contacts a processed product made from the plant, plant tissue, or plant cell, whereby the effector is delivered to the animal.

### D. Methods of Use in Insects, Mollusks, Fungi, or Nematodes

i. Delivery to Insects, Mollusks, Fungi, and Nematodes Via Plants

In some aspects, the disclosure features a method of delivering an effector to an insect, mollusk, fungus, or nematode, the method comprising providing to the insect, mollusk, fungus, or nematode a plant, plant tissue, or plant cell comprising a composition described herein (e.g., a composition comprising or consisting of a recombinant polynucleotide comprising (i) a ssRNA viroid sequence and (ii) a heterologous RNA sequence comprising or encoding an effector), wherein the insect, mollusk, fungus, or nematode consumes (e.g., ingests, digests, or absorbs) the plant, plant tissue, or plant cell or a part thereof, thereby taking up the effector and/or the recombinant polynucleotide. In embodiments, the effector is, e.g., any effector described in Section II herein.

In some aspects, the disclosure features a method of modulating a trait, phenotype, or genotype in an insect, mollusk, fungus, or nematode, the method comprising providing to the insect, mollusk, fungus, or nematode a plant, plant tissue, or plant cell comprising a composition described herein

In embodiments, the composition and/or effector is delivered to the organism (e.g., an insect, arachnid, fungus, mollusk, or nematode) by contacting the organism with a plant, plant part, or plant cell that has been provided with (e.g., contacted with) a composition comprising the recombinant peptide (e.g., as described in Section IIIA above), e.g., by ingestion, digestion, or absorption of all or a part of the plant, plant part, or plant cell by the insect, arachnid, fungus, mollusk, or nematode. For example, in embodiments, the composition is delivered by ingestion, digestion, or absorption of cytoplasm, plastids, xylem fluid, or phloem fluid by the insect, arachnid, fungus, mollusk, or nematode. In some embodiments, the compositions described herein are administered by providing at least one plant, plant part, or plant cell to which the composition has been delivered and on which the insect, mollusk, fungus, or nematode grows, lives, reproduces, or feeds. For example, in embodiments, the compositions are administered to a plant in an agricultural or horticultural environment. In some embodiments, the compositions described herein are administered by providing a plant, plant part, or plant cell to which the composition has been delivered as a food product for the insect, mollusk, fungus, or nematode, e.g., by including such a plant, plant part, or plant cell in a food product, growth media, or growth substrate.

In some aspects, the disclosure features an insect, arachnid, fungus, mollusk, or nematode comprising a recombinant polynucleotide of the disclosure (e.g., an ssRNA recombinant polynucleotide (e.g. a circular ssRNA) or a DNA molecule encoding such a polynucleotide).

Examples of insects, arachnids, fungi, mollusks, and nematodes that can be treated with the present compositions or related methods are further described herein.

ii. Delivery to fungi and methods of modifying fungi

In some aspects, the compositions described herein are delivered to fungi, e.g., beneficial fungal species or fungi that cause fungal diseases in plants. Beneficial fungal species include, but are not limited to edible fungi (e.g., mushrooms and truffles); fungi useful in leavening and fermentation (e.g., yeast); symbiotic fungi (e.g., mycorrhizal fungi); fungi used in decomposition; fungi used in bioremediation; and fungi used in manufacturing.

Beneficial fungal species include, but are not limited to Agaricus bisporus, Pieurotus species, Lentinula edodes, Auricularia auricula-judae, Volvariella volvacea, Flammulina velutpes, Tremella fucifbrmis, Hypsizygus tessellatus, Stropharia rugosoannulata, Cyclocybe aegerita, Hericium erinaceus, Boletus edulis, Calbovista subsculpta, Calvatia gigantea, Cantharellus cibarus, Craterellus tubaeformis, Cortinarus caperatus, Craterellus comucopioides, Grilbla ffondosa, Gyromitra esculenta, Hericium erinaceus, Hydnum repandum, Lactarius deliciosus, Morchella species, (e.g., Morchella conica var. deliciosa and Morchella esculenta var. rotunda), Pleurotus species, Tricholoma matsutake, Tuber species (e.g., Tuber aesfivum, Tuber bofchii, Tuber brumale, Tuber indicum, Tuber macrosporum, and Tuber mesentericum), Agaricus arvensis, Agaricus silvatcus, Amanita caesarea, Armillana mellea, Boletus badius, Calocybe gambosa, Calvatia utriformis, Chroogomphus species, Clavariaceae species, Clavulinaceae species, Coprinus comatus, Cortinarius variicolor, Cyttaria espinosae, Fistulina hepatica, Flammulina velutipes, Hygrophorus chrysodon, Kalaharituber pfeilii, Lactarius deterrmus, Lactarius salmonicolor, Lactarius subdulcis, Lactarius volemus, Laetiporus sulphureus, Leccinum aurantiacum, Leccinum scabrum, Leccinum versipelle, Macrolepiota procera, Marasmius oreades, Polyporus mylittae, Polyporus squamosus, Ramariaceae species, Rhizopogon luteolus, Russula species, Sparassis crispa, Suillus bovinus, Suillus ganulatus, Suillus luteus, Suillus tomentosus, Tricholoma terreum, Mucor hiemalis, Pleurots species, Pestalotopsis species, Aspergillus species, Phanerochaete chrysosporium, white rot mushrooms, Trichoderma species, and Saccharomyces species (e.g., Saccharomyces cerevisiae).In some aspects, the compositions described herein are useful for increasing the fitness of a fungus, e.g., a beneficial fungus.

In some aspects, the compositions described herein are useful for decreasing the fitness of a fungus, e.g., to prevent or treat a fungal infestation in a plant.

Fungal diseases include those caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator; diseases caused by rust disease pathogens, for example Gymnosporangium species, for example Gynnosporangium sabinae; Hemileia species, for example Hemileia vastatix; Phakopsora species, for example Phakopsora pachyrhizi and Phakopsora meibomiae; Puccinia species, for example Puccinia recondite, P. triticina, P. graminis or P. sriiformis or P. hordei; Uromyces species, for example Uromyces appendiculatus; diseases caused by pathogens from the group of the Oomycetes, for example Albugo species, for example Algubo candida; Bremia species, for example Bremia lactucae; Peronospora species, for example Peronospora pisi, P. parasitica or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium uldimum; leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidia form: Drechslera, Syn: Helminthosporium), Cochliobolus miyabeanus; Colletotrichum species, for example Colletotichumlinde muthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor, Glomerella species, for example Glomerella cingulata; Guignardia species, for example Guignardia bidwelli; Leptosphaeria species, for example Leptosphaeria maculans, Leptosphaeria nodorum; Magnaporthe species, for example Magnaporthe grisea; Microdochium species, for example Microdochium nivale; Mycosphaerella species, for example Mycosphaerella graminicola, M. arachidicola and M. fifiensis; Phaeosphaeria species, for example Phaeosphaeria nodorum; Pyrenophora species, for example Pyrenophora teres, Pyrenophora tritici repentis; Ramularia species, for example Ramularia colo-cygni, Ramularia areola; Rhynchosporium species, for example Rhynchosporium secalis; Septoria species, for example Septoria apii, Septoria lycopersii; Typhula species, for example Typhula incarnata; Ventura species, for example Ventura inaequalis; root and stem diseases caused, for example, by Corticium species, for example Corticium graminearum; Fusarium species, for example Fusarium oxysporum; Gaeumannomyces species, for example Gaeumannomyces graminis; Rhizoctonia species, such as, for example Rhizoctonia solani; Sarocadium diseases caused for example by Sarocadium oryzae; Sclerotium diseases caused for example by Sclerotium oryzae; Tapesia species, for example Tapesia aculbrmis; Thielaviopsis species, for example Thielaviopsis basicola; ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Septoria species, for example Septoria nodorum; diseases caused by smut fungi, for example Sphacelodeca species, for example Sphacelotheca reiliana; Tilletia species, for example Tilletia caries, T. controversa; Urocysdis species, for example Urocystis occulta; Ustilago species, for example Ustilago nuda, U. nuda tritici; fruit rot caused, for example, by Aspergillus species, for example Aspergillus flavus; Botrytis species, for example Botrytis cinerea; Penicillium species, for example Penicillium expansum and P. purpurogenum; Sclerotinia species, for example Sclerotinia sclerotiorum; Verlicilium species, for example Vericilium alboatrum; seed and soilborne decay, mould, wilt, rot and damping-off diseases caused, for example, by Alternaria species, caused for example by Alternaria brassicicola; Aphanomyces species, caused for example by Aphanomyces euteiches; Ascochyta species, caused for example by Ascochyta lens; Aspergillus species, caused for example by Aspergillus flavus; Cladosporium species, caused for example by Cladosporium herbarum; Cochliobolus species, caused for example by Cochliobolus sativus; (Conidiaform: Drechslera, Bipolaris Syn: Helminthosporium); Colletotrichum species, caused for example by Colletotrichum coccodes; Fusarium species, caused for example by Fusarium culmorum; Gibberella species, caused for example by Gibberella zeae; Macrophomina species, caused for example by Macrophomina phaseolina; Monographella species, caused for example by Monographella nivalis; Penicillium species, caused for example by Penicillium expansum; Phoma species, caused for example by Phoma lingam; Phomopsis species, caused for example by Phomopsis sojae; Phytophthora species, caused for example by Phytophthora cactorum; Pyrenophora species, caused for example by Pyrenophora graminea; Pyricularia species, caused for example by Pyricularia oryzae; Pythium species, caused for example by Pythium ultimum; Rhizoctonia species, caused for example by Rhizoctonia solani; Rhizopus species, caused for example by Rhizopus oryzae; Sclerotium species, caused for example by Sclerotium rolfsii; Septoria species, caused for example by Septoria nodorum; Typhula species, caused for example by Typhula incarnata; Verticillium species, caused for example by Verticillium dahliae; cancers, galls and witches' broom caused, for example, by Nectria species, for example Nectria galligena; wilt diseases caused, for example, by Monilinia species, for example Monilinia laxa; leaf blister or leaf curl diseases caused, for example, by Exobasidium species, for example Exobasidium vexans; Taphrina species, for example Taphrina deformans; decline diseases of wooden plants caused, for example, by Esca disease, caused for example by Phaemoniella clamydospora, Phaeoacremonium aleophilum and Fomitiporia mediterranea; Eutypa dyeback, caused for example by Eutypa lata; Ganoderma diseases caused for example by Ganoderma boninense; Rigidoporus diseases caused for example by Rigidoporus lignosus; diseases of flowers and seeds caused, for example, by Botrytis species, for example Botrytis cinerea; diseases of plant tubers caused, for example, by Rhizoctonia species, for example Rhizoctonia solani; Helminthosporium species, for example Helminthosporium solani; Club root caused, for example, by Plasmodiophora species, for example Plamodiophora brassicae; diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora. Fungal diseases further include diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolil), phyllostica leaf spot (Phyllosticta sojaecola), pod and stem blight (Phomopsis sojae), powdery mildew (Microsphaera diffusa), pyrenochaeta leaf spot (Pyrenochaeta glycines), Rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola). Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

In certain instances, the fungus is a Sclerotinia spp (Scelrotinia sclerotiorum). In certain instances, the fungus is a Botrytis spp (e.g., Botrytis cinerea). In certain instances, the fungus is an Aspergillus spp. In certain instances, the fungus is a Fusarium spp. In certain instances, the fungus is a Penicillium spp. Compositions of the present disclosure are useful in various fungal control applications. In embodiments, the above-described compositions are used to control fungal phytopathogens prior to harvest or post-harvest fungal pathogens. In one embodiment, any of the above-described compositions are used to control target pathogens such as Fusarium species, Botrytis species, Verticillium species, Rhizoctonia species, Trichoderma species, or Pythium species by applying the composition to plants. In another embodiment, compositions of the present disclosure are used to control post-harvest pathogens such as Penicillium, Geotrichum, Aspergillus niger, and Colletotrichum species.

Effectors that can be delivered to a fungus include any effector that has a biological effect on a fungus, e.g., a coding sequence (e.g., a protein or a polypeptide coding sequence) that has a biological effect on a fungus, a regulatory RNA (e.g., lncRNA, circRNA, tRF, tRNA, rRNA, snRNA, snoRNA, or piRNA) that has a biological effect on a fungus, an interfering RNA (e.g., a dsRNA, microRNA (miRNA), pre-miRNA, phasiRNA, hcsiRNA, or natsiRNA) that has a biological effect on a fungus, or a guide RNA that has a biological effect on a fungus (e.g., in combination with a gene editing enzyme). In some aspects, the effector binds a target host cell factor, e.g., a factor in or on a fungus cell, e.g., a nucleic acid (e.g., a DNA or an RNA) or a protein.

In instances in which the effector increases the fitness of the fungus (e.g., increases mobility, body weight, life span, fecundity, or metabolic rate of the fungus), in embodiments, the increase is, e.g., about 2%, 5%, 10%, 20%, 30%, 40%, 50%, 60%, 70%, 80%, 90%, 100%, or greater than 100% relative to a reference level (e.g., a level found in a host that does not receive a recombinant polynucleotide of the disclosure or an effector derived therefrom).

In instances in which the effector decreases the fitness of the fungus (e.g., decreases body weight, life span, fecundity, or metabolic rate of the fungus), in embodiments, the decrease is, e.g., about 2%, 5%, 10%, 20%, 30%, 40%, 50%, 60%, 70%, 80%, 90%, 100%, or greater than 100% relative to a reference level (e.g., a level found in a host that does not receive a recombinant polynucleotide of the disclosure or an effector derived therefrom). For example, in embodiments, the rate of death in a fungus population is increased by at least 10%, 20%, 30%, 40%, 50%, 60%, 70%, 80%, 90%, 100%, or greater than 100% relative to the reference level. Infestation of a plant by the fungus by about 2%, 5%, 10%, 20%, 30%, 40%, 50%, 60%, 70%, 80%, 90%, 100%, or greater than 100% relative to the reference level.

In instances in which the effector modulates or modifies a trait of the fungus (e.g., modulates expression of a gene), in embodiments, the modulation is an increase or a decrease of about 2%, 5%, 10%, 20%, 30%, 40%, 50%, 60%, 70%, 80%, 90%, 100%, or more than 100% relative to a reference level (e.g., a level found in a host that does not receive a recombinant polynucleotide of the disclosure or an effector derived therefrom).

iii. Delivery to Insects and Methods of Modifying Insects

In some aspects, the compositions described herein are delivered to invertebrates. Invertebrates of interest include invertebrates that are considered beneficial (e.g., pollinating insects, predatory insects that help to control invertebrate pests) or invertebrates that are domesticated for human use (e.g., European honey bee, Apis mellifera, silkworm, Bombyx mori, edible snails such as Helix spp.) and invertebrates that are considered pests or otherwise harmful. Invertebrate agricultural pests which damage plants, particularly domesticated plants grown as crops, include, but are not limited to, arthropods (e.g., insects, arachnids, myriopods), nematodes, platyhelminths, and molluscs. Important agricultural invertebrate pests include representatives of the insect orders coleoptera (beetles), diptera (flies), lepidoptera (butterflies, moths), orthoptera (grasshoppers, locusts), thysanoptera (thrips), and hemiptera (true bugs), arachnids such as mites and ticks, various worms such as nematodes (roundworms) and platyhelminths (flatworms), and molluscs such as slugs and snails. In some aspects, the compositions described herein are delivered to insects, e.g., beneficial insect species or insects that are pests, e.g., plant pests or animal pests. The term “insect” includes any organism belonging to the phylum Arthropoda and to the class Insecta or the class Arachnida, in any stage of development, i.e., immature and adult insects.

In some aspects, the compositions described herein are useful for increasing the fitness of an insect, e.g., a beneficial insect. Beneficial insects include, but are not limited to insects that participate in pollination (e.g., bees (e.g., honeybees), wasps, flies, beetles, butterflies, and moths) and insects that are involved in the generation of a commercial product (e.g., honeybees, silk worms, cochineal bugs, or insects used for food or animal feed).

In some aspects, the compositions described herein are useful for decreasing the fitness of an insect, e.g., to prevent or treat an insect infestation in a plant. Examples of agricultural insect pests include aphids, adalgids, phylloxerids, leafminers, whiteflies, caterpillars (butterfly or moth larvae), mealybugs, scale insects, grasshoppers, locusts, flies, thrips, earwigs, stinkbugs, flea beetles, weevils, bollworms, sharpshooters, root or stalk borers, leafhoppers, leafminers, and midges. Non-limiting, specific examples of important agricultural pests of the order Lepidoptera include, e.g., diamondback moth (Plutella xylostella), various “bollworms” (e.g., Diparopsis spp., Earias spp., Pectinophora spp., and Helicoverpa spp., including corn earworm, Helicoverpa zea, and cotton bollworm, Helicoverpa armigera), European corn borer (Ostrinia nubilalis), black cutworm (Agrotis ipsilon), “armyworms” (e.g., Spodoptera frugiperda, Spodoptera exigua, Spodoptera littoralis, Pseudaletia unipuncta), corn stalk borer (Papaipema nebris), Western bean cutworm (Striacosta albicosta), gypsy moths (Lymatria spp.), Pieris rapae, Pectinophora gossypiella, Synanthedon exitiosa, Melittia cucurbitae, Cydia pomonella, Grapholita molesta, Plodia interpunctella, Galleria mellonella, Manduca sexta, Manduca quinquemaculata, Lymantria dispar, Euproctis chrysorrhoea, Trichoplusia ni, Mamestra brassicae, Anticarsia gemmatalis, Pseudoplusia includens, Epinotia aporema, Heliothis virescens, Scripophaga incertulus, Sesamia spp., Buseola fusca, Cnaphalocrocis medinalis, and Chilo suppressalis. Non-limiting, specific examples of important agricultural pests of the order Coleoptera (beetles) include, e.g., Colorado potato beetle (Leptinotarsa decemlineata) and other Leptinotarsa spp., e.g., L. juncta (false potato beetle), L. haldemani (Haldeman's green potato beetle), L. lineolata (burrobrush leaf beetle), L. behrensi, L. collinsi, L. defecta, L. heydeni, L. peninsularis, L. rubiginosa, L. texana, L. tlascalana, L. tumamoca, and L. typographica; “corn rootworms” and “cucumber beetles” including Western corn rootworm (Diabrotica virgifera virgifera), Northern corn rootworm (D. barben), Southern corn rootworm (D. undecimpunctata howardi), cucurbit beetle (D. speciosa), banded cucumber beetle (D. balteata), striped cucumber beetle (Acalymma vittatum), and western striped cucumber beetle (A. trivittatum); “flea beetles”, e.g., Chaetocnema pulicaria, Phyllotreta spp., and Psylliodes spp.; “seedcorn beetles”, e.g., Stenolophus lecontei and Clivinia impressifrons; cereal leaf beetle (Oulema melanopus); Japanese beetles (Popillia japonica) and other “white grubs”, e.g., Phyllophaga spp., Cyclocephala spp.; khapra beetle (Trogoderma granartum); date stone beetle (Coccotrypes dactyliperda); boll weevil (Anthonomus grandis grandis); Dectes stem borer (Dectes texanus); “wireworms” “click beetles”, e.g., Melanotus spp., Agriotes mancus, and Limonius dubitans. Non-limiting, specific examples of important agricultural pests of the order Hemiptera (true bugs) include, e.g., brown marmorated stinkbug (Halyomorpha halys), green stinkbug (Chinavia hilaris); billbugs, e.g., Sphenophorus maidis; spittlebugs, e.g., meadow spittlebug (Philaenus spumarius); leafhoppers, e.g., potato leafhopper (Empoasca fabae), beet leafhopper (Circulifer tenellus), blue-green sharpshooter (Graphocephala atropunctata), glassy-winged sharp shooter (Homalodisca vitripennis), maize leafhopper (Cicadulina mbila), two-spotted leafhopper (Sophonia rufofascia), common brown leafhopper (Orosius orientalis), rice green leafhoppers (Nephotettix spp.), and white apple leafhopper (Typhlocyba pomaria); aphids (e.g., Rhopalosiphum spp., Aphis spp., Myzus spp.), grape phylloxera (Daktulosphaira vitifoliae), and psyllids, e.g., Asian citrus psyllid (Diaphorina citn), African citrus psyllid (Trioza erytreae), potato/tomato psyillid (Bactericera cockerelli). Other examples of important agricultural pests include thrips (e.g., Frankliniella occidentalis, F. tritici, Thrips simplex, T. palms); members of the order Diptera including Delia spp., fruitflies (e.g., Drosophila suzukii and other Drosophila spp., Ceratitis capitata, Bactrocera spp.), leaf miners (Liriomyza spp.), and midges (e.g., Mayetiola destructor).

Other invertebrates that cause agricultural damage include plant-feeding mites, e.g., two-spotted or red spider mite (Tetranychus urticae) and spruce spider mite (Oligonychus unungui); various nematode or roundworms, e.g., Meloidogyne spp., including M. incognita (southern root knot), M. enterlobii (guava root knot), M. javanica (Javanese root knot), M. hapla (northern root knot), and M. arenaria (peanut root knot), Longidorus spp., Aphelenchoides spp., Ditylenchus spp., Globodera rostochiensis and other Globodera spp., Nacobbus spp., Heterodera spp., Bursaphelenchus xylophilus and other Bursaphelenchus spp., Pratylenchus spp., Trichodorus spp., Xiphinema index, Xiphinema diversicaudatum, and other Xiphinema spp.; and snails and slugs (e.g., Deroceras spp., Vaginulus plebius, and Veronica leydigi).

Pest invertebrates also include those that damage human-built structures or food stores, or otherwise cause a nuisance, e.g., drywood and subterranean termites, carpenter ants, weevils (e.g., Acanthoscelides spp., Callosobruchus spp., Sitophilus spp.), flour beetles (Tribolium castaneum, Tribolium confusum) and other beetles (e.g., Stegobium paniceum, Trogoderma granarium, Oryzaephilus spp.), moths (e.g., Galleria mellonella, which damage beehives; Plodia interpunctella, Ephestia kuehniella, Tinea spp., Tineola spp.), silverfish, and mites (e.g., Acarus siro, Glycophagus destructor).

In related aspects, the compositions described herein are delivered to invertebrates that are considered human or veterinary pests, such as invertebrates that bite or parasitize humans or other animals, or that are vectors for disease-causing microbes (e.g., bacteria, viruses). Examples of these include dipterans such as biting flies and midges (e.g., Phlebotomus spp., Lutzomyia spp., Tabanus spp., Chrysops spp., Haematopota spp., Simulium spp.) and blowflies (screwworm flies) (e.g., Cochliomyia macellaria, C. hominivorax, C. aldrichi, and C. minima; also Chrysomya rufifacies and Chrysomya megacephala), tsetse fly (Glossina spp.), botfly (Dermatobia hominis, Dermatobia spp.); mosquitoes (e.g., Aedes spp., Anopheles spp., Culex spp., Culiseta spp.); bedbugs (e.g., Cimex lectularius, Cimex hemipterus) and “kissing bugs” (Triatoma spp.); members of the insect orders Phthiraptera (sucking lice and chewing lice, e.g., Pediculus humanus, Pthirus pubis) and Siphonaptera (fleas, e.g., Tunga penetrans). Parasitic arachnids also include important disease vectors; examples include ticks (e.g., Ixodes scapularis, Ixodes pacificus, Ixodes ricinus, Ixodes cookie, Amblyomma americanum, Amblyomma maculatum, Dermacentor variabilis, Dermacentor andersoni, Dermacentor albipictus, Rhipicephalus sanguineus, Rhipicephalus microplus, Rhipicephalus annulatus, Haemaphysalis longicornis, and Hyalomma spp.) and mites including sarcoptic mites (Sarcoptes scabiei and other Sarcoptes spp.), scab mites (Psoroptes spp.), chiggers (Trombicula alfreddugesi, Trombicula autumnalis), Demodex mites (Demodex folliculorum, Demodex brevis, Demodex canis), bee mites, e.g., Varroa destructor, Varroa jacobosoni, and other Varroa spp., tracheal mite (Acarapis woods), and Tropilaelaps spp. Parasitic worms that can infest humans and/or non-human animals include ectoparasites such as leeches (a type of annelid) and endoparasitic worms, collectively termed “helminths”, that infest the digestive tract, skin, muscle, or other tissues or organs. Helminths include members of the phyla Annelida (ringed or segmented worms), Platyhelminthes (flatworms, e.g., tapeworms, flukes), Nematoda (roundworms), and Acanthocephala (thorny-headed worms). Examples of parasitic nematodes include Ascaris lumbricoides, Ascaris spp., Parascaris spp., Baylisascaris spp., Brugia malayi, Brugia timori, Wuchereria bancrofti, Loa loa, Mansonella streptocerca, Mansonella ozzardi, Mansonella perstans, Onchocerca volvulus, Dirofilaria immitis and other Dirofilaria spp., Dracunculus medinensis, Ancylostoma duodenale, Ancyclostoma celanicum, and other Ancylostoma spp., Necator americanus and other Necator spp., Angriostrongylus spp., Uncinaria stenocephala, Bunostomum phlebotomum, Enterobius vermicularis, Enterobius gregorii, and other Enterobius spp., Strongyloides stercoralis, Strongyloides fuelleborni, Strongyloides papillosus, Strongyloides ransomi, and other Strongyloides spp., Thelazia californiensis, Thelazia callipaeda, Trichuris trichiura, Trichuris vulpis, Trichinella spiralis, Trichinella britovi, Trichinella nelson, Trichinella nativa, Toxocara canis, Toxocara cati, Toxascaris leonina, Wuchereria bancrofti, and Haemonchus contortus. Examples of parasitic platyhelminths include Taenia saginata, Taenia solium, Taenia multiceps, Diphyllobothrium latum, Echinococcus granulosus, Echinococcus multilocularis, Echinococcus vogeli, Echinococcus oligarthrus, Hymenolepis nana, Hymenolepis diminuta, Spirometra erinaceieuropaei, Schistosoma haematobium, Schistosoma mansoni, Schistosoma japonicum, Schistosoma intercalatum, Schistosoma mekongi, Fasciolopis buski, Heterophyes heterophyes, Fasciola hepatica, Fasciola gigantica, Clonorchis sinensis, Clonorchis vivirrini, Dicrocoelium dendriticum, Gastrodiscoides hominis, Metagonimus yokogawai, Metorchis conjunctus, Opisthorchis viverrine, Opisthorchis felineus, Paragonimus westermani, Paragonimus africanus, Paragonimus spp., Echinostoma echinatum, and Trichobilharzia regenti. Endoparasitic protozoan invertebrates include Axanthamoeba spp., Balamuthia mandrillaris, Babesia divergens, Babesia bigemina, Babesia equi, Babesia microfti, Babesia duncani, Balantidium coli, Blastocystis spp., Cryptosporidium spp., Cyclospora cayetanensis, Dientamoeba fragili, Entamoeba histolytica, Giardia lamblia, Isospora belli, Leishmania spp., Naegleria fowleri, Plasmodium falciparum, Plasmodium vivax, Plasmodium malariae, Plasmodium ovale curtisi, Plasmodium ovale wallikeri, Plasmodium knowlesi, Rhinosporidium seeberi, Sarcosystis spp., Toxoplasma gondii, Trichomonas vaginalis, Trypanosoma brucei, Trypanosoma cruzi.

In some aspects, the compositions described herein are suitable for preventing or treating infestation by an insect or other invertebrate, or a plant infested therewith, including insects belonging to the following orders: Acari, Araneae, Anoplura, Coleoptera, Collembola, Dermaptera, Dictyoptera, Diplura, Diptera (e.g., spotted-wing Drosophila), Embioptera, Ephemeroptera, Grylloblatodea, Hemiptera (e.g., aphids, Greenhouse whitefly), Homoptera, Hymenoptera, Isoptera, Lepidoptera, Mallophaga, Mecoptera, Neuroptera, Odonata, Orthoptera, Phasmida, Plecoptera, Protura, Psocoptera, Siphonaptera, Siphunculata, Thysanura, Strepsiptera, Thysanoptera, Trichoptera, or Zoraptera.

In some aspects, the insect is a Leptinotarsa species. In some aspects, the insect is Leptinotarsa decemlineata (Colorado potato beetle, CPB).

In some instances, the insect is from the class Arachnida, for example, Acarus spp., Aceria sheldoni, Aculops spp., Aculus spp., Amblyomma spp., Amphitetranychus viennensis, Argas spp., Boophilus spp., Brevipalpus spp., Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., Epitrimerus pyri, Eutetranychus spp., Eriophyes spp., Glycyphagus domesticus, Halotydeus destructor, Hemitarsonemus spp., Hyalomma spp., Ixodes spp., Latrodectus spp., Loxosceles spp., Metatetranychus spp., Neutrombicula autumnalis, Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., Tetranychus spp., Trombicula alfreddugesi, Vaejovis spp., or Vasates lycopersici.

In some instances, the insect is from the class Chilopoda, for example, Geophilus spp. or Scutigera spp.

In some instances, the insect is from the order Collembola, for example, Onychiurus armatus.

In some instances, the insect is from the class Diplopoda, for example, Blaniulus guttulatus; from the class Insecta, e.g. from the order Blattodea, for example, Blattella asahinai, Blattella germanica, Blatta orientalis, Leucophaea maderae, Panchlora spp., Parcoblatta spp., Periplaneta spp., or Supella longipalpa.

In some instances, the insect is from the order Coleoptera, for example, Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptolestes ferrugineus, Cryptorhynchus lapathi, Cylindrocopturus spp., Dermestes spp., Diabrotica spp. (e.g., corn rootworm), Dichocrocis spp., Dicladispa armigera, Diloboderus spp., Epilachna spp., Epitrix spp., Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., Lachnosterna consanguinea, Lasioderma serricorne, Latheticus oryzae, Lathridius spp., Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Necrobia spp., Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllophaga helleri, Phyllotreta spp., Popillia japonica, Premnotrypes spp., Prostephanus truncatus, Psylliodes spp., Ptinus spp., Rhizobius ventralis, Rhizopertha dominica, Sitophilus spp., Sitophilus oryzae, Sphenophorus spp., Stegobium paniceum, Sternechus spp., Symphyletes spp., Tanymecus spp., Tenebrio molitor, Tenebrioides mauretanicus, Tribolium spp., Trogoderma spp., Tychius spp., Xylotrechus spp., or Zabrus spp.

In some instances, the insect is from the order Diptera, for example, Aedes spp., Agromyza spp., Anastrepha spp., Anopheles spp., Asphondylia spp., Bactrocera spp., Bibio hortulanus, Calliphora erythrocephala, Calliphora vicina, Ceratitis capitata, Chironomus spp., Chrysomyia spp., Chrysops spp., Chrysozona pluvialis, Cochliomyia spp., Contarinia spp., Cordylobia anthropophaga, Cricotopus sylvestris, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., Lucilia spp., Lutzomyia spp., Mansonia spp., Musca spp. (e.g., Musca domestica), Oestrus spp., Oscinella fit, Paratanytarsus spp., Paralauterborniella subcincta, Pegomyia spp., Phlebotomus spp., Phorbia spp., Phormia spp., Piophila casei, Prodiplosis spp., Psila rosae, Rhagoletis spp., Sarcophaga spp., Simulium spp., Stomoxys spp., Tabanus spp., Tetanops spp., or Tipula spp.

In some instances, the insect is from the order Heteroptera, for example, Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptocorisa varicornis, Leptoglossus phyllopus, Lygus spp., Macropes excavatus, Miridae, Monalonion atratum, Nezara spp., Oebalus spp., Pentatomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., or Triatoma spp.

In some instances, the insect is from the order Homiptera, for example, Acizzia acaciaebaileyanae, Acizzia dodonaeae, Acizzia uncatoides, Acrida turrita, Acyrthosipon spp., Acrogonia spp., Aeneolamia spp., Agonoscena spp., Aleyrodes proletella, Aleurolobus barodensis, Aleurothrixus floccosus, Allocaridara malayensis, Amrasca spp., Anuraphis cardui, Aonidiella spp., Aphanostigma pini, Aphis spp. (e.g., Apis gossypii), Arboridia apicalis, Arytainilla spp., Aspidiella spp., Aspidiotus spp., Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., Cryptomyzus ribis, Cryptoneossa spp., Ctenarytaina spp., Dalbulus spp., Dialeurodes citri, Diaphorina citri, Diaspis spp., Drosicha spp., Dysaphis spp., Dysmicoccus spp., Empoasca spp., Eriosoma spp., Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Homalodisca vitripennis, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Macrosteles facifrons, Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nettigoniclla spectra, Nilaparvata lugens, Oncometopia spp., Orthezia praelonga, Oxya chinensis, Pachypsylla spp., Parabemisia myricae, Paratrioza spp., Parlatoria spp., Pemphigus spp., Pentatomidae spp. (e.g., Halyomorpha halys), Peregrinus maidis, Phenacoccus spp., Phloeomyzus passerinii, Phorodon humuli, Phylloxera spp., Pinnaspis aspidistrae, Planococcus spp., Prosopidopsylla flava, Protopulvinaria pyriformis, Pseudaulacaspis pentagona, Pseudococcus spp., Psyllopsis spp., Psylla spp., Pteromalus spp., Pyrilla spp., Quadraspidiotus spp., Quesada gigas, Rastrococcus spp., Rhopalosiphum spp., Saissetia spp., Scaphoideus titanus, Schizaphis graminum, Selenaspidus articulatus, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., Trialeurodes vaporariorum, Trioza spp., Typhlocyba spp., Unaspis spp., Viteus vitifolii, Zygina spp.; from the order Hymenoptera, for example, Acromyrmex spp., Athalia spp., Atta spp., Diprion spp., Hoplocampa spp., Lasius spp., Monomorium pharaonis, Sirex spp., Solenopsis invicta, Tapinoma spp., Urocerus spp., Vespa spp., or Xeris spp.

In some instances, the insect is from the order Isopoda, for example, Armadillidium vulgare, Oniscus asellus, or Porcellio scaber.

In some instances, the insect is from the order Isoptera, for example, Coptotermes spp., Cornitermes cumulans, Cryptotermes spp., Incisitermes spp., Microtermes obesi, Odontotermes spp., or Reticulitermes spp.

In some instances, the insect is from the order Lepidoptera, for example, Achroia grisella, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamstra brassicae, Melanitis leda, Mocis spp., Monopis obviella, Mythimna separata, Nemapogon cloacellus, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudaletia unipuncta, Pseudoplusia includens, Pyrausta nubilalis, Rachiplusia nu, Schoenobius spp., Scirpophaga spp., Scirpophaga innotata, Scotia segetum, Sesamia spp., Sesamia inferens, Sparganothis spp., Spodoptera spp., Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., Tryporyza incertulas, Tuta absoluta, or Virachola spp.

In some instances, the insect is from the order Orthoptera or Saltatoria, for example, Acheta domesticus, Dichroplus spp., Gryllotalpa spp., Hieroglyphus spp., Locusta spp., Melanoplus spp., or Schistocerca gregaria.

In some instances, the insect is from the order Phthiraptera, for example, Damalinia spp., Haematopinus spp., Linognathus spp., Pediculus spp., Ptirus pubis, Trichodectes spp.

In some instances, the insect is from the order Psocoptera for example Lepinatus spp., or Liposcelis spp.

In some instances, the insect is from the order Siphonaptera, for example, Ceratophyllus spp., Ctenocephalides spp., Pulex irritans, Tunga penetrans, or Xenopsylla cheopsis.

In some instances, the insect is from the order Thysanoptera, for example, Anaphothrips obscurus, Baliothrips biformis, Drepanothrips reuteri, Enneothrips flavens, Frankliniella spp., Heliothrips spp., Hercinothrips femoralis, Rhipiphorothrips cruentatus, Scirtothrips spp., Taeniothrips cardamoms, or Thrips spp.

In some instances, the insect is from the order Zygentoma (=Thysanura), for example, Ctenolepisma spp., Lepisma saccharina, Lepismodes inquilinus, or Thermobia domestica.

In some instances, the insect is from the class Symphyla, for example, Scutigerella spp.

In some instances, the “insect” (arachnid) is a mite, including but not limited to, Tarsonemid mites, such as Phytonemus pallidus, Polyphagotarsonemus latus, Tarsonemus bilobatus, or the like; Eupodid mites, such as Penthaleus erythrocephalus, Penthaleus major, or the like; Spider mites, such as Oligonychus shinkajii, Panonychus citri, Panonychus mori, Panonychus ulmi, Tetranychus kanzawai, Tetranychus urticae, or the like; Eriophyid mites, such as Acaphylla theavagrans, Aceria tulipae, Aculops lycopersici, Aculops pelekassi, Aculus schlechtendali, Eriophyes chibaensis, Phyllocoptruta oleivora, or the like; Acarid mites, such as Rhizoglyphus robini, Tyrophagus putrescentiae, Tyrophagus similis, or the like; Bee brood mites, such as Varroa jacobsoni, Varroa destructor or the like; Ixodides, such as Boophilus microplus, Rhipicephalus sanguineus, Haemaphysalis longicornis, Haemophysalis flava, Haemophysalis campanulata, Ixodes ovatus, Ixodes persulcatus, Amblyomma spp., Dermacentor spp., or the like; Cheyletidae, such as Cheyletiella yasguri, Cheyletiella blakei, or the like; Demodicidae, such as Demodex canis, Demodex cati, or the like; Psoroptidae, such as Psoroptes ovis, or the like; Scarcoptidae, such as Sarcoptes scabies, Notoedres cats, Knemidocoptes spp., or the like.

Table 5 shows further examples of insects that cause infestations that can be treated or prevented using the compositions and related methods described herein.

TABLE 5
Insect pests

| Common Name | Latin name |
|---|---|
| European corn borer | Ostrinia nubilalis |
| Corn earworm | Helicoverpa zea |
| Beet armyworm | Spodoptera exigua |
| Fall armyworm | Spodoptera frugiperda |
| Southwestern corn borer | Diatraea grandiosella |
| Lesser cornstalk borer | Elasmopalpus lignosellus |
| Stalk borer | Papaipema nebris |
| Common armyworm | Pseudaletia unipuncta |
| Black cutworm | Agrotis ipsilon |
| Western bean cutworm | Striacosta albicosta |
| Yellowstriped armyworm | Spodoptera ornithogalli |
| Western yellowstriped armyworm | Spodoptera praefica |
| Southern armyworm | Spodoptera eridania |
| Southern armyworm | Spodoptera eridania |
| Variegated cutworm | Peridroma saucia |
| Stalk borer | Papaipema nebris |
| Cabbage looper | Trichoplusia ni |
| Tomato pinworm | Keiferia lycopersicella |
| Tobacco hornworm | Manduca sexta |
| Tomato hornworm | Manduca quinquemaculata |
| Imported cabbageworm | Artogeia rapae |
| Cabbage butterfly | Pieris brassicae |
| Cabbage looper | Trichoplusia ni |
| Diamondback moth | Plutella xylostella |
| Beet armyworm | Spodoptera exigua |
| Common cutworm | Agrotis segetum |
| Potato tuberworm | Phthorimaea operculella |
| Diamondback moth | Plutella xylostella |
| Sugarcane borer | Diatraea saccharalis |
| Glassy cutworm | Crymodes devastator |
| Dingy cutworm | Feltia ducens |
| Claybacked cutworm | Agrotis gladiaria |
| Green cloverworm | Plathypena scabra |
| Soybean looper | Pseudoplusia includes |
| Velvetbean caterpillar | Anticarsia gemmatalis |
| Northern corn rootworm | Coleoptera Diabrotica barberi |
| Southern corn rootworm | Diabrotica undecimpunctata |
| Western corn rootworm | Diabrotica virgifera |
| Maize weevil | Sitophilus zeamais |
| Colorado potato beetle | Leptinotarsa decemlineata |
| Tobacco flea beetle | Epitrix hirtipennis |
| Crucifer flea beetle | Phyllotreta cruciferae |
| Western black flea beetle | Phyllotreta pusilla |
| Pepper weevil | Anthonomus eugenii |
| Colorado potato beetle | Leptinotarsa decemlineata |
| Potato flea beetle | Epitrix cucumeris |
| Wireworms Melanpotus spp. | Hemicrepidus memnonius |
| Wireworms | Ceutorhychus assimilis |
| Cabbage seedpod weevil | Phyllotreta cruciferae |
| Crucifer flea beetle | Melanolus spp. |
| Wireworm | Aeolus mellillus |
| Wheat wireworm | Aeolus mancus |
| Sand wireworm | Horistonotus uhlerii |
| Maize billbug | Sphenophorus maidis |
| Timothy bilibug | Sphenophorus zeae |
| Bluegrass billbug | Sphenophorus parvulus |
| Southern corn billbug | Sphenophorus callosus |
| White grubs | Phyllophaga spp. |
| Corn flea beetle | Chaetocnema pulicaria |
| Japanese beetle | Popillia japonica |
| Mexican bean beetle | Epilachna varivestis |
| Bean leaf beetle | Cerotoma trifurcate |
| Blister beetles | Epicauta pestifera |
| | Epicauta lemniscata |
| Corn leaf aphid | Homoptera Rhopalosiphum maidis |
| Corn root aphid | Anuraphis maidiradicis |
| Green peach aphid | Myzus persicae |
| Potato aphid | Macrosiphum euphorbiae |
| Greenhouse whitefly | Trileurodes vaporariorum |
| Sweetpotato whitefly | Bemisia tabaci |
| Silverleaf whitefly | Bemisia argentifolii |
| Cabbage aphid | Brevicoryne brassicae |
| Green peach aphid | Myzus persicae |
| Potato leafhopper | Empoasca fabae |
| Potato psyllid | Paratrioza cockerelli |
| Silverleaf whitefly | Bemisia argentifolii |
| Sweetpotato whitefly | Bemisia tabaci |
| Carrot aphid | Cavariella aegopodii |
| Cabbage aphid | Brevicoryne brassicae |
| West Indian canefly | Saccharosydne saccharivora |
| Yellow sugarcane aphid | Sipha flava |
| Threecornered alfalfa hopper | Spissistilus festinus |
| Lygus Hesperus | Hemiptera Lygus lineolaris |
| Lygus bug | Lygus rugulipennis |
| Green stink bug | Acrosternum hilare |
| Brown stick bug | Euschistus servus |
| Chinch bug | Blissus leucopterus leucopterus |
| Leafminer | Diptera Liriomyza trifolii |
| Vegetable leafminer | Liriomyza sativae |
| Tomato leafminer | Scrobipalpula absoluta |
| Seedcorn maggot | Delia platura |
| Cabbage maggot | Delia brassicae |
| Cabbage root fly | Delia radicum |
| Carrot rust fly | Psilia rosae |
| Sugarbeet root maggot | Tetanops myopaeformis |
| Differential grasshopper | Orthoptera Melanoplus differentialis |
| Redlegged grasshopper | Melanoplus femurrubrum |
| Twostriped grasshopper | Melanoplus bivittatus |

Effectors that can be delivered to an insect include any effector that has a biological effect on an insect, e.g., a coding sequence (e.g., a protein or a polypeptide coding sequence) that has a biological effect on an insect, a regulatory RNA (e.g., lncRNA, circRNA, tRF, tRNA, rRNA, snRNA, snoRNA, or piRNA) that has a biological effect on an insect, an interfering RNA (e.g., a dsRNA, microRNA (miRNA), pre-miRNA, phasiRNA, hcsiRNA, or natsiRNA) that has a biological effect on an insect, or a guide RNA that has a biological effect on a insect (e.g., in combination with a gene editing enzyme). In some aspects, the effector binds a target host cell factor, e.g., a factor in or on an arthropod cell, e.g., a nucleic acid (e.g., a DNA or an RNA) or a protein.

In instances in which the effector increases the fitness of the insect (e.g., increases mobility, body weight, life span, fecundity, or metabolic rate of the insect), in embodiments, the increase is, e.g., about 2%, 5%, 10%, 20%, 30%, 40%, 50%, 60%, 70%, 80%, 90%, 100%, or greater than 100% relative to a reference level (e.g., a level found in a host that does not receive a recombinant polynucleotide of the disclosure or an effector derived therefrom).

In instances in which the effector decreases the fitness of the insect (e.g., decreases body weight, life span, fecundity, or metabolic rate of the insect), in embodiments, the decrease is, e.g., about 2%, 5%, 10%, 20%, 30%, 40%, 50%, 60%, 70%, 80%, 90%, 100%, or greater than 100% relative to a reference level (e.g., a level found in a host that does not receive a recombinant polynucleotide of the disclosure or an effector derived therefrom). For example, in embodiments, the rate of death in an insect population is increased by at least 10%, 20%, 30%, 40%, 50%, 60%, 70%, 80%, 90%, 100%, or greater than 100% relative to the reference level. Infestation of a plant by the insect by about 2%, 5%, 10%, 20%, 30%, 40%, 50%, 60%, 70%, 80%, 90%, 100%, or greater than 100% relative to the reference level.

In instances in which the effector modulates a trait of the insect (e.g., modulates expression of a gene), in embodiments, the modulation is an increase or a decrease of about 2%, 5%, 10%, 20%, 30%, 40%, 50%, 60%, 70%, 80%, 90%, 100%, or more than 100% relative to a reference level (e.g., a level found in a host that does not receive a recombinant polynucleotide of the disclosure or an effector derived therefrom).

iv. Delivery to mollusks and methods of modifying mollusks

In some aspects, the compositions described herein are delivered to mollusks. The term “mollusk” includes any organism belonging to the phylum Mollusca.

In some aspects, the compositions described herein are suitable for preventing or treating infestation by terrestrial gastropods (e.g., slugs and snails) in agriculture and horticulture. They include all terrestrial slugs and snails which mostly occur as polyphagous pests on agricultural and horticultural crops. For example, in embodiments, the mollusk belongs to the family Achatinidae, Agriolimacidae, Ampullariidae, Arionidae, Bradybaenidae, Helicidae, Hydromiidae, Lymnaeidae, Milacidae, Urocyclidae, or Veronicellidae.

For example, in some instances, the mollusk is Achatina spp., Archachatina spp. (e.g., Archachatina marginata), Agriolimax spp., Anon spp. (e.g., A. ater, A. circumscriptus, A. distinctus, A. fasciatus, A. hortensis, A. intermedius, A. rufus, A. subfuscus, A. silvaticus, A. lusitanicus), Arliomax spp. (e.g., Ariolimax columbianus), Biomphalaria spp., Bradybaena spp. (e.g., B. fruticum), Bulinus spp., Cantareus spp. (e.g., C. asperses), Cepaea spp. (e.g., C. hortensis, C. nemoralis, C. hortensis), Cernuella spp., Cochlicella spp., Cochlodina spp. (e.g., C. laminata), Deroceras spp. (e.g., D. agrestis, D. empiricorum, D. laeve, D. panornimatum, D. reticulatum), Discus spp. (e.g., D. rotundatus), Euomphalia spp., Galba spp. (e.g., G. trunculata), Helicella spp. (e.g., H. itala, H. obvia), Helicigona spp. (e.g., H. arbustorum), Helicodiscus spp., Helix spp. (e.g., H. aperta, H. aspersa, H. pomatia), Limax spp. (e.g., L. cinereoniger, L. flavus, L. marginatus, L. maximus, L. tenellus), Limicolaria spp. (e.g., Limicolaria aurora), Lymnaea spp. (e.g., L. stagnalis), Mesodon spp. (e.g., Meson thyroidus), Monadenia spp. (e.g., Monadenia fidelis), Milax spp. (e.g., M. gagates, M. marginatus, M. sowerbyi, M. budapestensis), Oncomelania spp., Neohelix spp. (e.g., Neohelix albolabris), Opeas spp., Otala spp. (e.g., Otala lacteal), Oxyloma spp. (e.g., O. pfeiffen), Pomacea spp. (e.g., P. canaliculata), Succinea spp., Tandonia spp. (e.g., T. budapestensis, T. sowerbyi), Theba spp., Vallonia spp., or Zonitoides spp. (e.g., Z. nitidus).

In some aspects the compositions described herein are delivered to mollusks that are considered edible or otherwise beneficial. Such mollusks include those species cultivated for food or for other products (e.g., shells, pearls), including various species of clams, mussels, and oysters.

Effectors that can be delivered to a mollusk include any effector that has a biological effect on a mollusk, e.g., a coding sequence (e.g., a protein or a polypeptide coding sequence) that has a biological effect on a mollusk, a regulatory RNA (e.g., lncRNA, circRNA, tRF, tRNA, rRNA, snRNA, snoRNA, or piRNA) that has a biological effect on a mollusk, an interfering RNA (e.g., a dsRNA, microRNA (miRNA), pre-miRNA, phasiRNA, hcsiRNA, or natsiRNA) that has a biological effect on a mollusk, or a guide RNA that has a biological effect on a mollusk (e.g., in combination with a gene editing enzyme). In some aspects, the effector binds a target host cell factor, e.g., a factor in or on a mollusk cell, e.g., a nucleic acid (e.g., a DNA or an RNA) or a protein.

In instances in which the effector increases the fitness of the mollusk (e.g., increases mobility, body weight, life span, fecundity, or metabolic rate of the mollusk), in embodiments, the increase is, e.g., about 2%, 5%, 10%, 20%, 30%, 40%, 50%, 60%, 70%, 80%, 90%, 100%, or greater than 100% relative to a reference level (e.g., a level found in a host that does not receive a recombinant polynucleotide of the disclosure or an effector derived therefrom).

In instances in which the effector decreases the fitness of the mollusk (e.g., decreases body weight, life span, fecundity, or metabolic rate of the mollusk), in embodiments, the decrease is, e.g., about 2%, 5%, 10%, 20%, 30%, 40%, 50%, 60%, 70%, 80%, 90%, 100%, or greater than 100% relative to a reference level (e.g., a level found in a host that does not receive a recombinant polynucleotide of the disclosure or an effector derived therefrom). For example, in embodiments, the rate of death in a mollusk population is increased by at least 10%, 20%, 30%, 40%, 50%, 60%, 70%, 80%, 90%, 100%, or greater than 100% relative to the reference level. Infestation of a plant by the mollusk by about 2%, 5%, 10%, 20%, 30%, 40%, 50%, 60%, 70%, 80%, 90%, 100%, or greater than 100% relative to the reference level.

In instances in which the effector modulates a trait of the mollusk (e.g., modulates expression of a gene), in embodiments, the modulation is an increase or a decrease of about 2%, 5%, 10%, 20%, 30%, 40%, 50%, 60%, 70%, 80%, 90%, 100%, or more than 100% relative to a reference level (e.g., a level found in a host that does not receive a recombinant polynucleotide of the disclosure or an effector derived therefrom).

v. Delivery to Nematodes and Methods of Modifying Nematodes

In some aspects, the compositions described herein are delivered to a nematode. The compositions and related methods can be useful for decreasing the fitness of a nematode, e.g., to prevent or treat a nematode infestation in a plant. The term “nematode” includes any organism belonging to the phylum Nematoda.

The compositions and related methods are suitable for preventing or treating infestation by nematodes that cause damage plants including, for example, Meloidogyne spp. (root-knot), Heterodera spp., Globodera spp., Pratylenchus spp., Helicotylenchus spp., Radopholus similis, Ditylenchus dipsaci, Rotylenchulus reniformis, Xiphinema spp., Aphelenchoides spp. and Belonolaimus longicaudatus. In some instances, the nematode is a plant parasitic nematodes or a nematode living in the soil. Plant parasitic nematodes include, but are not limited to, ectoparasites such as Xiphinema spp., Longidorus spp., and Trichodorus spp.; semiparasites such as Tylenchulus spp.; migratory endoparasites such as Pratylenchus spp., Radopholus spp., and Scutellonema spp.; sedentary parasites such as Heterodera spp., Globodera spp., and Meloidogyne spp., and stem and leaf endoparasites such as Ditylenchus spp., Aphelenchoides spp., and Hirshmaniella spp. Especially harmful root parasitic soil nematodes are such as cystforming nematodes of the genera Heterodera or Globodera, and/or root knot nematodes of the genus Meloidogyne. Harmful species of these genera are, for example, Meloidogyne incognita, Heterodera glycines (soybean cyst nematode), Globodera pallida and Globodera rostochiensis (potato cyst nematode), which species are effectively controlled with the pest control (e.g., biopesticide or biorepellent) compositions described herein. However, the use of the pest control (e.g., biopesticide or biorepellent) compositions described herein is in no way restricted to these genera or species, but also extends in the same manner to other nematodes.

Other examples of nematodes that can be targeted by the methods and compositions described herein include but are not limited to, e.g., Aglenchus agricola, Anguina tritici, Aphelenchoides arachidis, Aphelenchoides fragaria and the stem and leaf endoparasites Aphelenchoides spp. in general, Belonolaimus gracilis, Belonolaimus longicaudatus, Belonolaimus nortoni, Bursaphelenchus cocophilus, Bursaphelenchus eremus, Bursaphelenchus xylophilus, Bursaphelenchus mucronatus, and Bursaphelenchus spp. in general, Cacopaurus pestis, Criconemella curvata, Criconemella onoensis, Criconemella ornata, Criconemella rusium, Criconemella xenoplax (=Mesocriconema xenoplax) and Criconemella spp. in general, Criconemoides femiae, Criconemoides onoense, Criconemoides ornatum and Criconemoides spp. in general, Ditylenchus destructor, Ditylenchus dipsaci, Ditylenchus myceliophagus and the stem and leaf endoparasites Ditylenchus spp. in general, Dolichodorus heterocephalus, Globodera pallida (=Heterodera pallida), Globodera rostochiensis (potato cyst nematode), Globodera solanacearum, Globodera tabacum, Globodera virginia and the sedentary, cyst forming parasites Globodera spp. in general, Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp. in general, Hemicriconemoides, Hemicycliophora arenaria, Hemicycliophora nudata, Hemicycliophora parvana, Heterodera avenae, Heterodera cruciferae, Heterodera glycines (soybean cyst nematode), Heterodera oryzae, Heterodera schachtii, Heterodera zeae and the sedentary, cyst forming parasites Heterodera spp. in general, Hirschmaniella gracilis, Hirschmaniella oryzae Hirschmaniella spinicaudata and the stem and leaf endoparasites Hirschmaniella spp. in general, Hoplolaimus aegyptii, Hoplolaimus califomicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp. in general, Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the sedentary parasites Meloidogyne spp. in general, Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp. in general, Paratylenchus hamatus, Paratylenchus minutus, Paratylenchus projectus and Paratylenchus spp. in general, Pratylenchus agilis, Pratylenchus alleni, Pratylenchus andinus, Pratylenchus brachyurus, Pratylenchus cerealis, Pratylenchus coffeae, Pratylenchus crenatus, Pratylenchus delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thornei, Pratylenchus vulnus, Pratylenchus zeae and the migratory endoparasites Pratylenchus spp. in general, Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp. in general, Rotylenchulus borealis, Rotylenchulus parvus, Rotylenchulus reniformis and Rotylenchulus spp. in general, Rotylenchus laurentinus, Rotylenchus macrodoratus, Rotylenchus robustus, Rotylenchus uniformis and Rotylenchus spp. in general, Scutellonema brachyurum, Scutellonema bradys, Scutellonema clathricaudatum and the migratory endoparasites Scutellonema spp. in general, Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp. in general, Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp. in general, Tylenchulus semipenetrans and the semiparasites Tylenchulus spp. in general, Xiphinema americanum, Xiphinema brevicolle, Xiphinema dimorphicaudatum, Xiphinema index and the ectoparasites Xiphinema spp. in general.

Other examples of nematode pests include species belonging to the family Criconematidae, Belonolaimidae, Hoploaimidae, Heteroderidae, Longidoridae, Pratylenchidae, Trichodoridae, or Anguinidae.

Table 6 shows further examples of nematodes, and diseases associated therewith, that can be treated or prevented using the compositions and related methods described herein.

TABLE 6
Nematode Pests

| Disease | Causative Agent |
|---|---|
| Awl | Dolichoderus spp., D. heterocephalus |
| Bulb and stem (Europe) | Ditylenchus dipsaci |
| Burrowing | Radopholus similes R. similis |
| Cyst | Heterodera avenae, H. zeae, H. |
| | schachti; Globodera |
| | rostochiensis, G. pallida, and G. |
| | tabacum; Heterodera trifolii, H. |
| | medicaginis, H. ciceri, H. |
| | mediterranea, H. cyperi, H. |
| | salixophila, H. zeae, H.goettingiana, H. |
| | riparia, H. humuli, H. latipons, H. |
| | sorghi, H. fici, H.litoralis, and H. |
| | turcomanica; Punctodera chalcoensis |
| Dagger | Xiphinema spp., X. americanum, X. |
| | Mediterraneum |
| False root-knot | Nacobbus dorsalis |
| Lance | Hoplolaimus spp., H. galeatus |
| Lance, Columbia | Hoplolaimus Columbus |
| Lesion | Pratylenchus spp., P. brachyurus, P. |
| | coffeae P. crenatus, P. hexincisus, P. |
| | neglectus, P. penetrans, P. scribneri, P. |
| | magnica, P. neglectus, P. thornei, P. |
| | vulnus, P. zeae |
| Needle | Longidorus spp., L. breviannulatus |
| Others | Hirschmanniella species, Pratylenchoid |
| | magnicauda |
| Ring | Criconemella spp., C. ornata |
| Root-knot | Meloidogyne spp., M. arenaria, M. |
| | chitwoodi, M. artiellia, M. fallax, M. |
| | hapla, M. javanica, M. incognita, M. |
| | microtyla, M. partityla, M. |
| | panyuensis, M, paranaensis |
| Spiral | Helicotylenchus spp. |
| Sting | Belonolaimus spp., B. longicaudatus |
| Stubby-root | Paratrichodorus spp., P. christiei, P. |
| | minor, Quinisulcius acutus, |
| | Trichodorus spp. |
| Stunt | Tylenchorhynchus dubius |

Effectors that can be delivered to a nematode include any effector that has a biological effect on a nematode, e.g., a coding sequence (e.g., a protein or a polypeptide coding sequence) that has a biological effect on a nematode, a regulatory RNA (e.g., lncRNA, circRNA, tRF, tRNA, rRNA, snRNA, snoRNA, or piRNA) that has a biological effect on a nematode, an interfering RNA (e.g., a dsRNA, microRNA (miRNA), pre-miRNA, phasiRNA, hcsiRNA, or natsiRNA) that has a biological effect on a nematode, or a guide RNA that has a biological effect on a nematode (e.g., in combination with a gene editing enzyme). In some aspects, the effector binds a target host cell factor, e.g., a factor in or on a nematode cell, e.g., a nucleic acid (e.g., a DNA or an RNA) or a protein.

In instances in which the effector increases the fitness of the nematode (e.g., increases mobility, body weight, life span, fecundity, or metabolic rate of the nematode), in embodiments, the increase is, e.g., about 2%, 5%, 10%, 20%, 30%, 40%, 50%, 60%, 70%, 80%, 90%, 100%, or greater than 100% relative to a reference level (e.g., a level found in a host that does not receive a recombinant polynucleotide of the disclosure or an effector derived therefrom).

In instances in which the effector decreases the fitness of the nematode (e.g., decreases body weight, life span, fecundity, or metabolic rate of the nematode), in embodiments, the decrease is, e.g., about 2%, 5%, 10%, 20%, 30%, 40%, 50%, 60%, 70%, 80%, 90%, 100%, or greater than 100% relative to a reference level (e.g., a level found in a host that does not receive a recombinant polynucleotide of the disclosure or an effector derived therefrom). For example, in embodiments, the rate of death in a nematode population is increased by at least 10%, 20%, 30%, 40%, 50%, 60%, 70%, 80%, 90%, 100%, or greater than 100% relative to the reference level. Infestation of a plant by the nematode by about 2%, 5%, 10%, 20%, 30%, 40%, 50%, 60%, 70%, 80%, 90%, 100%, or greater than 100% relative to the reference level.

In instances in which the effector modulates a trait of the nematode (e.g., modulates expression of a gene), in embodiments, the modulation is an increase or a decrease of about 2%, 5%, 10%, 20%, 30%, 40%, 50%, 60%, 70%, 80%, 90%, 100%, or more than 100% relative to a reference level (e.g., a level found in a host that does not receive a recombinant polynucleotide of the disclosure or an effector derived therefrom).

### EXAMPLES

The following are examples of the methods of the disclosure. It is understood that various other embodiments can be practiced, given the general description provided above.

Table of Contents

| Example | Title |
|---|---|
| Example 1 | In vitro transcription of linear RNAs |
| Example 2 | Production of circular RNA using splint ligation |
| Example 3 | Production of circular RNA using bacteria transfected with a tRNA ligase |
| Example 4 | Production of circular RNA using ligation of ribozyme-cleaved ends |
| Example 5 | RNA circularization efficiency |
| Example 6 | Circularized RNA is circular and not concatemeric |
| Example 7 | Reduced degradation susceptibility in circular RNAs |
| Example 8 | Isolation and purification of circular RNAs |
| Example 9 | Delivery of circular RNA to plant protoplasts |
| Example 10 | Delivery of circular RNA via leaf rubbing |
| Example 11 | Delivery of circular RNA via leaf infiltration |
| Example 12 | ELVd with a hairpin RNA targeting a tomato gene |
| Example 13 | ELVd with a guide RNA targeting a corn gene |
| Example 14 | PSTVd with a small RNA targeting a tomato gene |
| Example 15 | PSTVd TL-R or PSTVd R with a pre-miRNA targeting a tomato gene |
| Example 16 | Arabidopsis circRNA with a PSTVd replication motif |
| Example 17 | In planta viral trafficking motifs conjugated to a fluorescent aptamer as a circular |
| | fusion RNA |
| Example 18 | PSTVd with a Spinach RNA aptamer in Arabidopsis and corn |
| Example 19 | An RNA motif to traffic aptamers to a subcellular location |
| Example 20 | An RNA motif to traffic pre-miRNA to a specific cell type |
| Example 21 | An RNA motif to traffic an aptamer to a specific tissue type |
| Example 22 | Circular RNAs with PSTVd LT and EMCV or maize HSP101 IRES in monocots |
| | vs. dicots |
| Example 23 | PSTVd trafficking loop, replication loop, and Spinach RNA aptamer |
| Example 24 | An RNA vector containing two RNA motifs from two different viroid sources to |
| | deliver an aptamer to a specific cell type |
| Example 25 | In planta circularization of viral trafficking motifs conjugated to a fluorescent |
| | aptamer through transfection of a linear fusion RNA |
| Example 26 | PSTVd R + dsRNA to target RPL7 in CBP |
| Example 27 | A nuclear transporter based on PSTVd delivers RNA cargo to nucleus in |
| | Nicotiana tabacum protoplasts |

### Example 1. In Vitro Transcription of Linear RNAs

This Example describes in vitro transcription of RNAs using the 17 RNA polymerase promoter. In embodiments, this method is used to produce linear RNAs, such as the linear RNAs described in this specification.

Linear RNAs useful as circular RNA precursors were synthesized as follows: linear, 5′-mono phosphorylated in vitro transcripts were generated using the HiScuibe™ 17 Quick High Yield RNA Synthesis Kit (New England BloLabs®3 Inc., REF: E2050S). In vitro transcription was performed according to the manufacturer's protocol. Around 40 μg of linear RNA was generated in each reaction. After incubation, each reaction was treated with DNase to remove the DNA template. Linear RNA ribonucleotides were then column-purified using the Zymo RNA Clean & Concentrator-5 kit (Zymo Research: R1014). Linear transcribed ribonucleotides were quality tested by heating in vitro transcription products to 80° C. for 7-10 minutes. Heated ribonucleotides were then run on an agarose gel to validate purity of transcribed RNA and RNA quality. The expected bands of the appropriate molecular weight were observed by gel electrophoresis.

A modified procedure was also carried out as follows: Linear RNAs useful as circular RNA precursors were synthesized as follows: linear, 5′-mono phosphorylated in vitro transcripts were generated using the Lucigen AmpliScribe T7-Flash kit (ASF3507). In vitro transcription was performed according to the manufacturer's protocol. Around 100 μg of linear RNA was generated in each reaction. After incubation, each reaction was treated with DNase to remove the DNA template. Linear RNA ribonucleotides were then column-purified using the Monarch 500 RNA Clean-up Kit (T2050L). Linear transcribed ribonucleotides were quality tested by denaturing and/or native gel electrophoresis. Denaturing gel electrophoresis was performed by dissolving in vitro transcription products in gel loading buffer with a final concentration of greater than or equal to 50% formamide; heating at 95° C. for 3 minutes and cooling rapidly to 4° C., followed by running on urea PAGE gel with TBE running buffer. Native gel electrophoresis was performed by heating in vitro transcription products in water at 95° C. for 3 minutes; cooling slowly to room temperature for at least 15 minutes; and running on agarose gel with a voltage of less than 100V until bands were resolved. The expected bands of the appropriate molecular weight were observed by gel electrophoresis.

### Example 2. Production of Circular RNA Using Splint Ligation

Non-naturally occurring circular RNAs can be engineered to include one or more desirable properties, and can be produced using recombinant DNA technology. This Example describes in vitro production of circular RNA from linear RNA using splint ligation. In embodiments, this method is used to circularize linear RNAs described herein or other linear RNAs useful in the methods and compositions described herein.

In this Example, a general protocol for generating the circular RNA is as follows: DNA templates for in vitro transcription are amplified from a plasmid comprising a sequence of interest. Amplified DNA templates are gel-purified with a DNA gel purification kit (Qiagen). 250-500 ng of purified DNA template is subjected to in vitro transcription.

Linear, 5′-mono phosphorylated in vitro transcripts are generated using T7 RNA polymerase from each DNA template having corresponding sequences in the presence of 7.5 mM guanosine monophosphate (GMP), 1.5 mM guanosine triphosphate (GTP), 7.5 mM uracil triphosphate (UTP), 7.5 mM cytosine triphosphate (CTP), and 7.5 mM adenosine triphosphate (ATP). Around 40 μg of linear RNA is generated in each reaction. After incubation, each reaction is treated with DNase to remove the DNA template. The in vitro transcribed RNA is precipitated with ethanol in the presence of 2.5M ammonium acetate to remove unincorporated monomers.

Transcribed linear RNA is circularized using T4 RNA ligase 2 on a 20 nt splint DNA oligomer as template. Splint DNA is designed to anneal between 5-25 nucleotides (nt) of each 5′ or 3′ end of the linear RNA, leaving 2 nt at each end of the RNA unpaired. After annealing with the splint DNA (3 μM), 1 μM linear RNA is incubated with 0.5 U/μl T4 RNA ligase 2 at 37° C. for 4 hours. A mixture without T4 RNA ligase 2 is used as a negative control.

The circularization of linear RNA is monitored by separating RNA on 6% denaturing PAGE. Because of their circular structure, circular RNAs migrate more slowly than linear RNAs on denaturing polyacrylamide gels. The addition of ligase to the RNA mixtures generates new bands that appear above the linear RNA bands that are present in the mixtures that lack ligase ((−) lanes). Slower-migrating bands appear in all RNA mixtures containing ligase, indicating that successful splint ligation (e.g., circularization) occurred for multiple constructs, but not for the negative control.

In one experiment, circular RNA was generated as follows: DNA templates for in vitro transcription were amplified from a plasmid with corresponding sequences with a T7 promoter-harboring forward primer and a 2-O-methylated nucleotide with a reverse primer. Amplified DNA templates were column-purified using DNA Clean and Concentrator 5 kit (Zymo Research). Linear, 5′-mono phosphorylated in vitro transcripts were generated from purified DNA template (250-500 ng) using T7 RNA polymerase in the presence of 7.5 mM guanosine monophosphate (GMP), 1.5 mM guanosine triphosphate (GTP), 7.5 mM uracil triphosphate (UTP), 7.5 mM cytosine triphosphate (CTP), and 7.5 mM adenosine triphosphate (ATP). Around 40 μg of linear RNA was generated in each reaction. After incubation, each reaction was treated with DNase to remove the DNA template. The in vitro transcribed RNA was precipitated with ethanol in the presence of 2.5 M ammonium acetate to remove unincorporated monomers.

Transcribed linear RNA was circularized using T4 RNA ligase 2 on a 20-nucleotide (nt) splint DNA oligomer as template. Splint DNA was designed to anneal to 10 nt of the 5′ end of the linear RNA and to 10 nt of the 3′ end of the linear RNA, leaving 2 nt at each end of the linear RNA unpaired. After annealing with the splint DNA (3 μM), 1 μM linear RNA was incubated with 0.5 U/μl T4 RNA ligase 2 at 37° C. for 4 hours. A mixture without T4 RNA ligase 2 was used as a negative control. The circularization of linear RNA was monitored by separating RNA on 6% denaturing PAGE. The addition of ligase to the RNA mixtures generated new bands that appeared at higher apparent molecular weight (˜10 kb) than the linear RNA bands (˜1 kb) that were observed in the negative control lanes (mixtures without ligase). Slower-migrating bands (˜10 kb apparent molecular weight) appeared in all RNA mixtures containing ligase, indicating that successful splint ligation (i.e., circularization) occurred in all the reactions including the ligase, but not in the negative control reactions lacking the ligase.

In another experiment, the circular RNA was generated as follows: DNA templates for in vitro transcription were amplified from a plasmid with corresponding sequences with a T7 promoter-harboring forward primer and reverse primer with 5′ terminal nucleotide corresponding to the 3′ terminal nucleotide of the desired RNA. Amplified DNA templates were column purified using DNA Clean and Concentrator 5 kit (Zymo Research). 250-500 ng of purified DNA template was subjected to in vitro transcription. Linear, 5′-tri-phosphorylated in vitro transcripts were generated using T7 RNA polymerase from each DNA template having corresponding sequences. Around 100 ug of linear RNA was generated in each reaction. After incubation, each reaction was treated with DNase to remove the DNA template. Linear RNA ribonucieotides were then column-purified using the Monarch 500 RNA Clean-up Kit (T2050L). Linear 5′-triphosphorylated RNAs were converted to linear 5′-mono-phosphorylated RNAs using pyrophosphohydrolase enzyme RppH (New England Biolabs, M0356S) according to the manufacturer's instructions. Linear 5′ mono-phosphorylated RNAs were column purified using the Monarch 500 RNA Clean-up Kit (T2050L).

Transcribed linear RNA was circularized using T4 RNA ligase 2 (New England Biolabs) using a 30 nt splint DNA oligomer as template. The splint DNA was designed to anneal to 15 nt of the 5′ end of the linear RNA and to 15 nt of the 3′ end of the linear RNA, leaving no unpaired nt at either end of the linear RNA. After annealing with the splint DNA (3 μM), 1 μM linear RNA was incubated with 0.5 U/μl T4 RNA ligase 2 at 37° C. for 4 hours. The circularization of linear RNA was monitored by separating RNA on 6% denaturing PAGE. The addition of ligase to the RNA mixtures generated new bands that appeared at higher apparent molecular weight (˜10 kb) than the linear RNA bands (˜1 kb) that were observed in the negative control lanes (mixtures without ligase). Slower-migrating bands (˜10 kb apparent molecular weight) appeared in all RNA mixtures containing ligase, indicating that successful splint ligation (i.e., circularization) occurred in all the reactions including the ligase, but not in the negative control reactions lacking the ligase.

### Example 3. Production of Circular RNA Using Bacteria Transfected with a tRNA Ligase

ELVd are plant pathogens consisting of a single-stranded circular RNA that replicates in host cells and is circularized by endogenous tRNA ligases. This Example describes production of circular RNA in a model bacterial system (E. coli) using co-expression of Eggplant Latent Viroid (ELVd) RNA as an exemplary RNA and eggplant tRNA ligase as an exemplary tRNA ligase; any tRNA ligase can be used to circularize a viroid RNA. The fluorescent Spinach RNA aptamer (Huang et al., Nat Chem Biol, 10: 686-691, 2014) is used as a model effector. In some embodiments, this method is used to circularize other linear RNAs useful in the methods and compositions described herein.

As shown in the following Example, significant quantities of an aptamer-containing circular RNA were generated in E. coli, as described by Daros et al., Scientific Reports, 8: Article Number 1904, 2018. Briefly, plasmids were constructed using standard molecular cloning techniques, with a first plasmid containing an ELVd sequence with a Spinach RNA aptamer insertion and a second plasmid containing a sequence coding for eggplant tRNA ligase. The E. coli strains BL21(DE3) (Novagen) and DH5-Alpha (New England BioLabs® Inc.) were transformed or co-transformed with one or both plasmids, and recombinant clones were selected at 37° C. on LB solid medium plates (10 g/L tryptone, 5 g/L yeast extract, 10 g/L NaCl and 1.5% agar) including the appropriate antibiotics (50 μg/mL ampicillin, 34 μg/mL chloramphenicol, or both). E. coli containing the plasmids of interest were grown in liquid cultures in Terrific Broth (TB) medium (12 g/L tryptone, 24 g/L yeast extract, 0.4% glycerol, 0.17 M KH₂PO₄ and 0.72 M K₂HPO₄), containing the appropriate antibiotics (see above), at 37° C. with shaking (225 revolutions per minute (rpm)). Cell densities were measured by absorbance at 600 nm with a spectrophotometer (Implen OD600 DiluPhotometer).

At the desired time points, 2 mL aliquots of the liquid cultures were taken and cells were sedimented by centrifuging at 13,000 rpm for 2 minutes. Cells were resuspended in 50 μL of TE buffer (10 mM Tris-HCl, pH 8.0 and 1 mM EDTA) by vortexing. One volume (50 μL) of a 1:1 (v/v) mix of phenol (saturated with water and equilibrated at pH 8.0 with Tris-HCl, pH 8.0) and chloroform were added, and the cells were broken by vigorous vortexing. The aqueous and organic phases were separated by centrifugation for 5 minutes at 13,000 rpm. The aqueous phases were recovered and re-extracted with one volume (50 μL) of chloroform. The aqueous phases containing total bacterial nucleic acids were finally recovered by pipetting.

Total RNA from E. coli was analyzed by denaturing PAGE. Twenty μL of RNA preparations were mixed with 1 volume (20 μL) of loading buffer (98% formamide, 10 mM Tris-HCl, pH 8.0, 1 mM EDTA, 0.0025% bromophenol blue and 0.0025% xylene cyanol), heated for 1.5 minutes at 95° C., and snap cooled on ice. Electrophoresis was run for 2.5 hours at 200 V in 6% urea polyacrylamide gels, or 1 hour at 200 V in 10% urea polyacrylamide gels. Electrophoresis buffer was 1×TBE without urea. Gels were stained by shaking for 15 minutes in 200 mL of 1 μg/mL ethidium bromide. This allowed visualization of the separation of circular RNA from linear RNA. Slower-migrating bands appeared in all RNA mixtures, indicating successful circularization in the bacterial cells.

To assay the fluorescence of Spinach RNA aptamer, aliquots of the E. coli cultures are supplemented with 200 μM DFHBI and grown for one additional hour. Pelleted bacteria are photographed under a stereomicroscope (Leica MZ16 F, Leica Microsystems) with UV illumination and a GFP2 filter (Leica Microsystems). To assay the fluorescence of Spinach RNA aptamer in RNA extracts, the extracts were run on denaturing PAGE gels as described above. After running, gels were washed 3× in water for 5 minutes. Gels were then incubated in 10 ml aptamer buffer (10 mM MgCl₂, 50 mM Na-HEPES (pH 7.5), 100 mM KC) containing 10 μM of the fluorophore (5Z)-5-[(3,5-Difluoro-4-hydroxyphenyl)methylene]-3,5-dihydro-2,3-dimethyl-4H-Imidazol-4-one, (Z)-4-(3,5-Difluoro-4-hydroxybenzylidene)-1,2-dimethyl-1H-imidazol-5(4H)-one (“DFHBI”, which fluoresces when bound to the Spinach aptamer, Sigma, SML1627-5MG) for 30 minutes with gentle agitation. Gels were briefly washed with water and then imaged using the Invitrogen iBright Imaging System with 470 nm excitation and 503 nm emission. Gels were then stained with ethidium bromide and imaged as described above. Composite images of DFHBI and ethidium bromide were generated. This gel electrophoresis and visualization revealed a band specifically stained by DFHBI, which migrated according to the known migration of circular ELVd polyribonucleotides. This band was only observed in RNA mixtures derived from cells containing heterologously expressed eggplant tRNA ligase, and confirmed the eggplant tRNA ligase-mediated circularization of the ELVd molecule containing the Spinach RNA aptamer.

### Example 4. Production of Circular RNA Using Ligation of Ribozyme-Cleaved Ends

This Example describes production of circular RNA using ligation of ribozyme-cleaved ends. The fluorescent Broccoli RNA aptamer is used as a model effector. In embodiments, this method is used to circularize other linear RNAs useful in the methods and compositions described herein.

As shown in the following Example, ribozyme-cleaved ends of linear RNAs can be joined to synthesize circular RNA, as described in Litke and Jaffrey, Nature Biotechnology, 37: 667-675, 2019. Recently described “Twister” ribozymes undergo self-cleavage to produce 5′ hydroxyl and 2′,3′-cylic phosphate ends. These ends are recognized for ligation by the E. coli RNA ligase RtcB. To trigger RNA circularization, RNA transcripts are expressed containing an RNA of interest flanked by ribozymes that undergo spontaneous autocatalytic cleavage. The resulting RNA contains 5′ and 3′ ends that are then ligated by the nearly ubiquitous endogenous RNA ligase RtcB, thereby producing circular RNAs. DNA templates containing a Broccoli fluorogenic RNA aptamer sequence a 5′ P3 Twister U2A ribozyme, and a 3′ P1 Twister ribozyme are prepared, and RNA is synthesized as described in Example 1. RNA is gel-purified as described below in Example 8.

After gel purification of autocatalytically cleaved RNA, 300 pmol of the purified RNA are treated with T4 Polynucleotide Kinase (New England Biolabs® Inc.) according to the manufacturer's protocol at 37° C. for 30 minutes. The enzyme is then inactivated for 20 minutes at 65° C. The products are cleaned by phenol chloroform extraction using heavy phase-lock tubes (Quantabio 2302830). 10 pmol of the gel-purified RNA is ligated using RtcB Ligase (New England Biolabs® Inc. M0458) for 1 hour at 37° C. Total RNA (1.0-2.5 μg) is separated using precast 6% or 10% TBE-Urea Gels (Life Technologies EC68655), and run at 270 V in TBE buffer until completion. Gels are washed 3×5 minutes with water and then stained for 30 minutes in 10 μM DFHBI in buffer prepared at room temperature containing 40 mM HEPES pH 7.4, 100 mM KCl, 1 mM MgCl₂. Broccoli RNA aptamer bands are then imaged using a ChemiDoc MP (Bio-Rad) with 470/30 nm excitation and 532/28 nm emission. Gels are washed additionally with water and stained with SYBR™ Gold (ThermoFisher Scientific, S11494) diluted in TBE buffer. RNA bands are then imaged using a ChemiDoc MP (Bio-Rad) with a preset channel (302 nm excitation and 590/110 nm emission). Gel band intensities are quantified in Image Lab 5.0 software (Bio-Rad).

In one experiment, DNA templates containing a MangoIII or Spinach fluorogenic RNA aptamer sequence, a 5′ P3 Twister U2A ribozyme, and a 3′ P1 Twister ribozyme were prepared, and RNA was synthesized as described in Example 1. RNA was gel-purified as described in Example 8.

After gel purification of autocatalytically cleaved RNA, 200 pmol of the gel-purified RNA was ligated using RtcB Ligase (New England Biolabs® Inc. M0458) for 1 hour at 37° C. RNA mixtures were optionally subjected to exonuclease treatment as described in Example 7 to preferentially degrade linear RNAs. Total RNA (1.0-2.5 μg) were separated using precast 6% or 10% TBE-Urea Gels (Life Technologies EC68655) and run at 270 V in TBE buffer until completion. Gels were washed thrice for 5 minutes each time with water and then stained for 30 minutes in 10 μM TO1-Biotin (for Mango III) or 10 μM DFHBI (for Spinach) in buffer prepared at room temperature containing 40 mM HEPES pH 7.4, 100 mM KCl, 1 mM MgCl₂. Mango III or Spinach RNA aptamer bands were then imaged using a ChemiDoc MP (Bio-Rad) with 470/30 nm excitation and 532/28 nm emission. Gels were washed additionally with water and stained with SYBR™ Gold (ThermoFisher Scientific, S11494) diluted in TBE buffer. RNA bands were then imaged using a ChemiDoc MP (Bio-Rad) with a preset channel (302 nm excitation and 590/110 nm emission). Gel band intensities were quantified in Image Lab 5.0 software (Bio-Rad).

Gel electrophoresis and imaging revealed bands that stained brightly with TO1-Biotin and DFHBI, corresponding to Mango III RNA aptamer-containing RNAs and Spinach RNA aptamer-containing RNAs, respectively. RNA mixtures treated with RtcB ligase had differential migration compared with the no-ligase negative control, confirming the circular nature of the RNA molecules formed by the ligation reaction. When these ligase-treated RNA mixtures were subjected to exonuclease treatment, the resulting RNA mixtures still contained the putative circular bands upon electrophoresis, while the linear bands from the non-ligase-treated mixture were degraded upon exonuclease treatment and not observed upon electrophoresis. These observations confirmed the ligase-mediated circularization of aptamer-containing RNAs.

In a separate experiment, DNA templates containing a tobacco PDS gene sequence, a 5′ P3 Twister U2A ribozyme, and a 3′ P1 Twister ribozyme were prepared, and RNA was synthesized as described in Example 1. RNA was gel-purified as described in Example 8.

After gel purification of autocatalytically cleaved RNA, 200 pmol of the gel-purified RNA was ligated using RtcB Ligase (New England Biolabs® Inc. M0458) for 1 hour at 37° C. RNA mixtures were optionally subjected to exonuclease treatment as described in Example 7 to preferentially degrade linear RNAs. Total RNA (1.0-2.5 μg) were separated using precast 6% or 10% TBE-Urea Gels (Life Technologies EC68655) and run at 250 V in TBE buffer until completion. Gels were washed thrice for 5 minutes each time with water and then stained with ethidium bromide. RNAs were then imaged using iBright imager. Gel electrophoresis and imaging revealed different populations in each RNA mixture. RNA mixtures treated with RtcB ligase had differential migration compared with the no-ligase control, confirming the circular nature of the resulting molecules. When these ligase-treated RNA mixtures were subjected to exonuclease treatment, the resulting RNA mixtures still contained the putative circular bands upon electrophoresis, while the linear bands from the non-ligase-treated mixture were degraded upon exonuclease treatment and not observed upon electrophoresis. This confirmed the ligase-mediated circularization of RNAs with ribozyme-cleaved ends.

### Example 5. RNA Circularization Efficiency

This Example describes measurement of circularization efficiencies for the methods described in Examples 2-4. In some embodiments, this method is used to assess circularization of any RNA.

To measure circularization efficiency, linear RNA transcripts are generated as described in Example 1 and circularized using the methods described in Examples 2-4. The circular RNAs are resolved by 6% denaturing PAGE, and RNA bands on the gel corresponding to linear or circular RNA are excised for purification. Excised RNA gel bands are crushed and RNA is eluted with 800 μl of 300 mM NaCl overnight. Gel debris is removed by centrifuge filters, and RNA is precipitated with ethanol in the presence of 0.3M sodium acetate.

Alternatively, images of gels can be recorded and band intensities analyzed using ImageJ. Intensities of each band can be normalized to a standard curve of RNA with known concentration, such as a dilution series or molecular weight ladder. This can serve as a proxy for RNA amount for any given band. Circularization efficiency is calculated as follows: the amount of eluted circular RNA is divided by the total eluted RNA amount (circular+linear RNA).

In one experiment, a modified version of the method described above for the measurement of circularization efficiencies was employed. In embodiments, this method may be used to assess circularization of an RNA.

To measure circularization efficiency, linear RNA transcripts were generated as described in Example 1 and circularized using the methods described in Examples 2-4. Four circular RNAs (of 683, 709, 790, or 1026 nt, respectively) were resolved by 6% denaturing PAGE and stained for 5 minutes with 1 μg/mL ethidium bromide. Images of gels were recorded and band intensities analyzed using ImageJ. Intensities of each band were recorded. Linear RNAs migrated at the expected molecular weight (˜600 nt to ˜1 kb), while circular molecules of the corresponding RNA migrated at higher apparent molecular weight (˜8 kb to ˜10 kb). Circularization efficiency was computed by dividing the intensity of the circular RNA band by the sum of the intensities of all bands in a given lane. Circularization efficiencies for the four RNAs were 78% (683 nt), 62% (709 nt), 75% (790 nt), and 65% (1026 nt), respectively.

### Example 6. Circularized RNA is Circular and not Concatemeric

This Example describes degradation of putative circular RNAs by RNAse H, which produces nucleic acid degradation products consistent with a circular and not a concatemeric RNA, thereby confirming that the RNAs are circular. In embodiments, this method is used to assess circularization of any RNA.

When incubated with a ligase, RNA can (i) not react, (ii) form an intramolecular bond, generating a circular (no free ends) RNA, or (iii) form an intermolecular bond, generating a concatemeric RNA. Treatment of each type of RNA with a complementary DNA oligomer and RNAse H, a nonspecific endonuclease that recognizes DNA/RNA duplexes, is expected to produce a unique number of degradation products of specific sizes depending on the RNA material which is tested.

To test circularization status of the RNA, 0.05 pmol/μl of linear or circular RNA is incubated with 0.25 U/μl of RNAse H, an endoribonuclease that digests DNA/RNA duplexes, and 0.3 pmol/μl DNA oligomer against a region of the RNA at 37° C. for 20 minutes. After incubation, the reaction mixture is analyzed by 6% denaturing PAGE.

For a linear RNA, it is expected that after binding of the DNA oligomer and subsequent cleavage by RNAse H, two cleavage products are obtained. A concatemer is expected to produce at least three cleavage products. A circular RNA is expected to produce a single cleavage product. This is visualized as the presence of one, two or three bands on the denaturing PAGE gel.

### Example 7. Reduced Degradation Susceptibility in Circular RNAs

Circular RNA is more resistant to exonuclease degradation than linear RNA due to the lack of 5′ and 3′ ends. This Example describes reduced susceptibility of circular RNA to degradation by an exonuclease compared to linear RNA. RNAse R is used as a model exonuclease. In embodiments, this method is used to assess degradation susceptibility of any RNA.

Circular RNA is generated and circularized as described in Examples 2-4 for use in the assay. To test circularization, 20 ng/μl of linear or circular RNA is incubated with 2 U/μl of RNAse R, a 3′ to 5′ exoribonuclease that digests linear RNAs but does not digest lariat or circular RNA structures, at 37° C. for 30 minutes. After incubation, the reaction mixture is analyzed by 6% denaturing PAGE.

The linear RNA bands present in the lanes lacking exonuclease are absent in the circular RNA lane, indicating that circular RNA shows higher resistance to exonuclease treatment as compared to a linear RNA control.

In one experiment, circular RNA was generated and circularized as described in Examples 1-4 for use in the assay. To test circularization, 260 ng ELVd RNA containing both circular and linear ELVd RNA was incubated with 10 U RNase R (Lucigen) (a 3′ to 5′ exoribonuclease that digests linear RNAs but does not digest lariat or circular RNA structures) and Terminator 5′-phosphate-dependent exoribonuclease (Lucigen). After incubation, the reaction mixture was column purified with 10 μg Monarch RNA Cleanup Kit (New England Biolabs) and analyzed by 6% denaturing PAGE. A low-range ssRNA ladder (New England Biolabs) was used as reference. The linear RNA bands at ˜300 bases that were present in the samples that had not been treated with exonuclease had much higher intensity in comparison to the corresponding bands from samples that had been treated with exonuclease. In contrast, the circular RNA bands at ˜500 bases that were present in the samples that had been treated with exonuclease had comparable intensity with that of the corresponding bands from samples not treated with exonuclease. This demonstrated that the circular RNAs had higher resistance to exoribonuclease treatment as compared to linear RNAs.

### Example 8. Isolation and Purification of Circular RNAs

This Example describes purification of circular RNAs. In some embodiments, this method is used to purify any circular RNA.

In certain embodiments, circular RNAs, as described in the previous Examples, are ligated, isolated from linear precursors, and purified before use in protoplasts or plants. This Example describes isolation of circular RNAs using UREA denaturing polyacrylamide gel separation.

Circular RNAs are synthesized as described in Examples 2-4. To purify the circular RNAs, ligation mixtures are resolved on 6% denaturing PAGE, and RNA bands corresponding to each of the circular RNAs are excised. Excised RNA gel fragments are crushed, and RNA is eluted with 800 μl of 300 mM NaCl overnight at 4° C. Gel debris is removed by filtration, and RNA is precipitated with ethanol in the presence of 0.3M sodium acetate. Eluted circular RNA is analyzed by 6% denaturing PAGE.

In one experiment, circular RNA was ligated and isolated from linear precursors using denaturing polyacrylamide (PAGE) gel separation in a protocol adapted from Nilsen, T. W. (2013). Gel purification of RNA. Cold Spring Harbor Protocols, 2013(2), pdb-prot072942. The resulting purified circular RNA is suitable for use in protoplasts or plants.

Circular RNAs were synthesized as described in Examples 2-4. To purify the circular RNAs, ligation mixtures were column purified using Monarch RNA cleanup kit (New England Biolabs) and resolved on 6% denaturing PAGE. RNA bands corresponding to each of the circular RNAs were excised and placed in a microcentrifuge tube with 400 μL of gel elution buffer (20 mM Tris-HCL, 0.25 M sodium acetate, 1 mM EDTA, 0.25% SDS), frozen on dry ice for 15 minutes, and incubated at room temperature overnight. (Alternatively, excised RNA gel fragments can be crushed using gel breaker tubes (IST Engineering Inc.), frozen on dry ice for 15 minutes, and incubated with gel elution buffer at room temperature for 6 hours or at 37° C. for 4 hours.) Gel debris was removed by microcentrifugation at maximum speed for 10 minutes at room temperature. RNA was then extracted from the supernatant with 1 volume of UltraPure phenol:chloroform:isoamyl alcohol (25:24:1, v/v) (Invitrogen), followed by i volume of chloroform. The RNA was precipitated with 2 volumes of ethanol and 1 μL glycogen (Thermo Scientific, v/v, 20 mg/ml) per mL of RNA/ethanol mixture, and eluted with RNase-free water. Eluted circular RNA was analyzed by 6% denaturing PAGE.

### Example 9. Delivery of Circular RNA to Plant Protoplasts

This Example describes the production of plant protoplasts and delivery of circular RNA to protoplasts using polyethylene glycol (PEG).

As a model circular RNA, circular RNA containing an ELVd sequence with a Spinach RNA aptamer insertion is synthesized, isolated and purified as described in Examples 3 and 5. The method can be used to deliver other circular RNAs to protoplasts.

Arabidopsis thaliana and Zea mays are used as a model dicot and monocot, respectively. Protoplasts can be prepared from any plant, e.g., any dicot or monocot.

Arabidopsis thaliana and Zea mays plants are grown from seed for 3-4 weeks and 8-10 days, respectively. Protoplasts are prepared from them as described below.

a. Monocot Mesophyll Protoplasts

The following mesophyll protoplast preparation protocol is generally suitable for use with monocot plants, e.g., maize (Zea mays) and rice (Oryza sativa):

An enzyme solution containing 0.6 molar mannitol, 10 millimolar MES pH 5.7, 1.5% cellulase R-10, and 0.3% macerozyme R-10 was prepared. The enzyme solution was heated at 50-55 degrees Celsius for 10 minutes to inactivate proteases and facilitate R10 enzyme activity, and cooled to room temperature before adding 1 millimolar CaCl₂, 5 millimolar beta-mercaptoethanol, and 0.1% bovine serum albumin. The enzyme solution was passed through a 0.45 micrometer filter. Washing solution containing 0.6 molar mannitol, 4 millimolar MES pH 5.7, and 20 millimolar KCl was prepared.

Second leaves of the plant were obtained, and the middle 6-8 centimeters are cut out. Ten leaf sections were stacked and cut into 0.5 millimeter-wide strips without bruising the leaves. The leaf strips were completely submerged in the enzyme solution in a petri dish, covered with aluminum foil, and incubated between 2-3 hours with gentle agitation. After digestion, the enzyme solution containing protoplasts was carefully transferred using a serological pipette through a 35 micrometer nylon mesh into a round-bottom tube; the petri dish was rinsed with 5 milliliters of washing solution and filtered through the mesh as well. The protoplast suspension was centrifuged at 1200 rpm, for 2 minutes in a swing-bucket centrifuge. The supernatant was aspirated without touching the pellet; the pellet was gently washed once with 20 milliliters washing buffer, and the supernatant was removed carefully. The pellet was resuspended by gently swirling in a small volume of washing solution, then resuspended in 10-20 milliliters of washing buffer. The tube was placed upright on ice for 30 minutes-4 hours (no longer). After resting on ice, the supernatant was removed by aspiration and the pellet resuspended with 2-5 milliliters of washing buffer. The concentration of protoplasts was measured using a hemocytometer and the concentration was adjusted to 2×10⁵ protoplasts/milliliter with washing buffer.

b. Dicot Mesophyll Protoplasts

The following mesophyll protoplast preparation protocol (modified from one described by Niu and Sheen, Methods Mol. Bio., 876:195-206, 2012) is generally suitable for use with dicot plants such as Arabidopsis thaliana and brassicas such as kale (Brassica oleracea):

An enzyme solution containing 0.4 M mannitol, 20 millimolar KCl, 20 millimolar MES pH 5.7, 1.5% cellulase R-10, and 0.4% macerozyme R-10 was prepared and heated at 50-55° C. for 10 minutes to inactivate proteases and facilitate R-10 enzyme activity. It was then cooled it to room temperature before adding 10 millimolar CaCl₂, 5 millimolar β-mercaptoethanol, and 0.1% bovine serum albumin, then passed through a 0.45 micrometer filter. A “W5” solution (154 millimolar NaCl, 125 millimolar CaCl₂, 5 millimolar KCl, and 2 millimolar MES at pH 5.7) and a “MMg solution” solution (0.4 molar mannitol, 15 millimolar MgC12, and 4 millimolar MES at pH 5.7) were prepared.

The second or third pair of true leaves of the plant were obtained, and the middle section was cut. 4-8 leaf sections were stacked and cut into 0.5 millimeter wide strips without bruising the leaves. The leaf strips were submerged completely in the enzyme solution contained in a petri dish, covered with aluminum foil, and incubated for 2-3 hours with gentle agitation. After digestion, the enzyme solution containing protoplasts was carefully transferred using a serological pipette through a 35 micrometer nylon mesh into a round-bottom tube; the petri dish was rinsed with 5 milliliters of washing solution and filtered through the mesh as well. The protoplast suspension was centrifuged at 1200 rpm, 2 minutes in a swing-bucket centrifuge. The supernatant was aspirated without touching the pellet; the pellet was then gently washed once with 20 milliliters washing buffer, and the supernatant was removed carefully afterwards. The pellet was gently resuspended by swirling in a small volume of washing solution, then resuspended in 10-20 milliliters of washing buffer. The tube was placed upright on ice for 30 minutes-4 hours (no longer). After resting on ice, the supernatant was removed by aspiration and the pellet resuspended with 2-5 milliliters of MMg solution.

c. Preparation of Arabidopsis and Maize Protoplasts

Plant protoplasts were prepared as described by Niu and Sheen, Plant Signalling Networks, 196-206, 2011. Briefly, Arabidopsis thaliana and Zea mays plants were grown from seed for 3-4 weeks, and well-expanded leaves were selected. The leaf tip was removed (3 mm) and the middle part of the leaf was cut into 0.5-1 mm strips. Leaf strips were transferred to the enzyme solution containing: 0.4 M mannitol, 20 mM KCl, 20 mM MES, pH 5.7, 1.5% cellulase R10 (Yakult Pharmaceutical Ind. Co., Ltd., Japan), 0.4% macerozyme R10 (Yakult Pharmaceutical Ind. Co., Ltd., Japan), 10 mM CaCl₂), 1 mM b-mercaptoethanol, and 0.1% BSA. The petri dish was covered with aluminum foil and incubated for 2-3 hours with gentle agitation. The digestion time may vary depending on the material and experimental goals.

Protoplasts (monocot and dicot) were PEG transfected as described by Niu and Sheen, Plant Signalling Networks, 196-206, 2011. Briefly, protoplast cells were allowed to settle at the bottom of the tube and the W5 solution was pipetted out. The protoplast pellet was resuspended in MMg solution (0.4 M mannitol, 15 mM MgCl₂, 4 mM MES, pH 5.7) to a final concentration of 2×10⁵/ml. 10 μl (10-20 ug) of circular RNA or linear control RNA, 100 μl of protoplasts in MMg solution, and 110 μl of PEG solution (40% (w/v) of PEG 4000 (Sigma-Aldrich), 0.2 M mannitol, and 0.1 M CaCl₂) were incubated at room temperature for 5-10 minutes. 440 μl of W5 solution was added and gently mixed by inverting to stop the transfection. The protoplasts were then pelleted by spinning at 110×g for 1 minute, and the supernatant was removed. The protoplasts were gently resuspended with 500 μl of WI solution (0.5 M mannitol, 4 mM MES, pH 5.7, 20 mM KC) in each well of a 12-well tissue culture plate and incubated for 24 and 48 hours.

d. Confirmation of Transfection

Presence of the transfected RNA in protoplasts can be confirmed using RNA extraction and quantitative RT-PCR. RNA extraction can be performed with the Maxwell® RSC simplyRNA Blood Kit (Promega; AS1380) or RNAzol® RT (MRC). Quantitative RT-PCR can be employed on samples from different time points to measure level of circular RNA or linear control in protoplasts.

In instances in which the transfected RNA comprises or encodes a fluorescent moiety, presence of the target RNA in protoplasts can be confirmed using microscopy. To assay the fluorescence of RNA aptamer Spinach, protoplast aliquots are supplemented with 200 μM DFHBI and incubated for 1 additional hour. Pelleted protoplasts are photographed under a stereomicroscope (Leica MZ16 F) with UV illumination and a GFP2 filter (Leica). In the case of the RNA extracts, DFHBI is directly added to 20 μM of the RNA extract and photographed under the same conditions.

The presence of fluorescently labelled RNA can also be detected by microscopy. In one experiment, RNA including Cy3-fluorescently labeled UTPs was transfected into maize protoplasts. The transfected protoplasts were centrifuged at 110×g for 1 minute and the supernatant was removed. The protoplasts were gently washed with WI solution, centrifuged at 110×g for 1 minute, and the supernatant removed. The protoplasts were then resuspended in 200 μL of WI; 20 μL of this protoplast suspension was imaged with an inverted fluorescent microscope (Olympus IXplore Standard), and the bright field and RFP filter images (at 10× or 40× magnification) were merged to provide a composite image. This allowed identification of a localized Cy3 signal in protoplast cells, confirming a positive transfection.

### Example 10. Delivery of Circular RNA Via Leaf Rubbing

This Example describes delivery of circular RNA to a plant via leaf rubbing. As a model circular RNA, circular RNA containing an ELVd sequence with a Spinach RNA aptamer insertion is synthesized, isolated and purified as described in Examples 3 and 5. In embodiments, this method is used to deliver other circular RNAs. Arabidopsis thaliana and Zea mays are used as a model dicot and monocot, respectively. In embodiments, leaf rubbing is used to deliver circular RNA to any plant, e.g., any dicot or monocot.

Arabidopsis thaliana and Zea mays plants are grown from seed for 4 weeks. Circular RNA is diluted to a concentration of 10 μg/ml and delivered to leaves via rubbing as described by Hull, Current Protocols in Microbiology, 13(1): 16B.6.1-16B.6.4, 2009. Briefly, wearing a glove, the forefinger is wetted with the RNA solution and wiped gently onto one marked leaf. Alternatively, a glass spatula is used. In embodiments, the leaf rubbing protocol includes carborundum dusting. Ten minutes after rubbing, inoculated leaves are washed with water from a squeeze bottle. Plants are then placed in a growth chamber and incubated for 1, 2, 7 and 14 days.

To detect the total level of RNA, quantitative reverse transcriptase PCR (RT-qPCR) is performed on inoculated leaf, non-inoculated leaf, root and stem of plants from all groups. Spinach RNA aptamer levels are quantified using fluorescence microscopy as described in Example 3 and expressed as arbitrary units of fluorescence (a.u.f).

In one experiment, circular RNA, including a 21-nt sequence (as a mature miRNA or siRNA or alternatively as a miRNA precursor encoding the 21-nt mature miRNA) designed to silence the Nicodiana benthamiana phytoene desaturase (PDS) gene, was produced in-vitro via transformed E. coli. The total RNA from the transformed E. coli was extracted using a hot phenol RNA extraction method. Nicotiana benthamiana were grown from seed for 4 weeks. The extracted RNA was diluted to a concentration of 25 ng/μL of total RNA (estimated to be about 10% circular RNA), and 100 μL of this RNA solution was delivered to each true leaf via rubbing as described by Hull, Current Protocols in Microbiology, 13(1): 16B.6.1-16B.6.4, 2009, with modification. Briefly, a forefinger of a gloved hand was wetted with the RNA solution and wiped gently onto a single marked leaf in the presence of powdered carborundum. One hour after rubbing, inoculated leaves were washed with millQ water from a squeeze bottle. Plants were then placed in a growth chamber and incubated under 16:8 light:dark cycle. Samples from each of four leaves per treatment condition were taken 8 days post-inoculation and RNA extracted from each leaf individually. Suppression of the target gene as an indicator of circular RNA delivery was assessed by measuring PDS levels with quantitative reverse transcriptase PCR (RT-qPCR) (two RT-qPCR reactions per RNA extraction). Samples from leaves treated with circular RNA molecules including either pre-miRNA or miRNA as cargos were compared to samples taken from a water-treated (negative) control. PDS levels in all samples were normalized to levels of the Nicodiana tabacum 60S ribosomal protein L23a-like reference gene (GENBank ID: 107805175). Results are provided in Table 7; decreased amounts of the PDS target gene were observed in samples from leaves treated with either of the circular RNAs, indicating that these circular RNAs including a pre-miRNA or a mature miRNA suppressed the PDS target gene.

TABLE 7
Results

| | Mean | | |
|---|---|---|---|
| | Percent | Standard | Relative |
| Sample | Suppression | Deviation | Comparison |
| Circular pre- | 14.4% | 49.4% | Circular pre-miRNA |
| miRNA | | | to Water Negative |
| Circular miRNA | 57.2% | 13.4% | Circular pre-miRNA |
| | | | to Water Negative |
| Water (negative) | −1.6% | 19.2% | Water Negative to |
| Control | | | Water Negative |

### Example 11. Delivery of Circular RNA Via Leaf Infiltration

This Example describes delivery of circular RNA to a plant via leaf infiltration.

As a model circular RNA, circular RNA containing ELVd sequence with a Spinach RNA aptamer insertion is synthesized, isolated, and purified as described in Examples 3 and 5. In embodiments, this method is used to deliver other circular RNAs. Arabidopsis thaliana and Zea mays are used as a model dicot and monocot, respectively. Leaf infiltration can be used to deliver circular RNA to any plant, e.g., any dicot. Arabidopsis thaliana and Zea mays plants are grown from seed for 4 weeks. Circular RNA is diluted to a concentration of 10 μg/ml and delivered to leaves via infiltration as described by Leuzinger et al., Journal of Visualized Experiments, 77: 50521, 2013. Briefly, 100 μl of the prepared RNA is loaded into a syringe without a needle and a small nick is made with the needle in the epidermis on the back side of a marked leaf.

Then, taking a firm hold of the front side of the leaf and applying gentle counter pressure to the nick with the thumb of one hand, the RNA solution is injected into the nick with the needle-less syringe. The injection is continued into the nick until the darker green circle indicating infiltration stops expanding. Another nick is made, and the injection repeated until the entire leaf is infiltrated and the whole leaf turns darker green. Plants are then placed in a growth chamber and incubated for 1, 2, 7 and 14 days.

To detect the total level of RNA, RT-qPCR is performed on inoculated leaf, non-inoculated leaf, root and stem of plants from all groups. Spinach RNA aptamer levels are quantified using fluorescence microscopy as described in Example 3 and expressed as a.u.f.

In one experiment, circular RNA containing ELVd wild-type sequence was synthesized, isolated, and purified as described in Examples 3 and 5, and infiltrated into Nicodiana benthamiana. Nicodiana benthamiana plants were grown from seed for 4 weeks. Circular RNA was diluted to a concentration of 3 mg/mL and delivered to leaves via infiltration as described by Leuzinger et al., Journal of Visualized Experiments, 77: 50521, 2013. Briefly, 100 μL of the prepared RNA was loaded into a syringe without a needle and a small nick was made with the needle in the epidermis on the back side of a marked leaf. The front side of the leaf was firmly held and a gentle pressure counter to the pressure of the nick was applied to infiltrate the RNA solution into the nick with the needle-less syringe. Infiltration was continued into the nick until the darker green circle indicating infiltration stopped expanding. Another nick was made, and the injection was repeated until the entire leaf was infiltrated and the whole leaf turned darker green. Plants were then placed in a growth chamber and incubated for hours.

An RT-qPCR assay specific to circular ELVd molecules was used to measure total circular RNA from leaf-disc samples taken from circular ELVd-infiltrated, compared to samples taken from water-infiltrated (negative control) leaves, and normalized to levels of the Nicotiana tabacum 60S ribosomal protein L23a-like reference gene (GENBank ID: 107805175). Furthermore, the measured amounts of circular ELVd were normalized to total RNA, and estimated circular ELVd copy number was determined with a standard curve containing known copy numbers of circular ELVd. Results are provided in Table 8.

TABLE 8
Results

| | Average | | | |
|---|---|---|---|---|
| | Circular ELVd | | | |
| | Fold Change | | | |
| | (ELVd-infiltrated | | Average | |
| Hours after | vs. water- | Standard | Copy Number | Standard |
| infiltration | infiltrated) | Deviation | Circular ELVd | Deviation |
| 0 | 8.4 × 10\hat{ }4 | 1.2 × 10\hat{ }4 | 3.0 × 10\hat{ }8 | 2.2 × 10\hat{ }7 |
| 24 | 1.5 × 10\hat{ }3 | 2.6 × 10\hat{ }2 | 3.5 × 10\hat{ }7 | 7.9 × 10\hat{ }6 |

### Example 12. ELVd with a Hairpin RNA Targeting a Tomato Gene

This example describes the modification of a viroid to produce and deliver an RNA molecule that includes an effector to a plant and change its phenotype.

In this example, the RNA vector includes the following:
ELVd (Eggplant Latent Viroid) (SEQ ID NO: 1)
Effector: hpRNA-SIPDS: hairpin RNA (SEQ ID NO: 2) targeting a tomato (Solanum lycopersicum; SI) endogenous gene, PDS (phytoene desaturase)

The hpRNA targeting SIPDS (SEQ ID NO: 2) is derived from virus-induced gene silencing (VIGS) PDS RNAi design (Liu et al., The Plant Journal, 31(6): 777-786, 2002), with a 9 nt loop region replacing the original Flaveria trinervia Pyruvate orthophosphate DiKinase 2 (PDK) intron and 40 nt targeting PDS exons. The hairpin (hp) RNA is inserted in U245-U246 of ELVd. The RNA vector (SEQ ID NO: 3) is synthesized in a bacterial viroid system as described in Example 3.

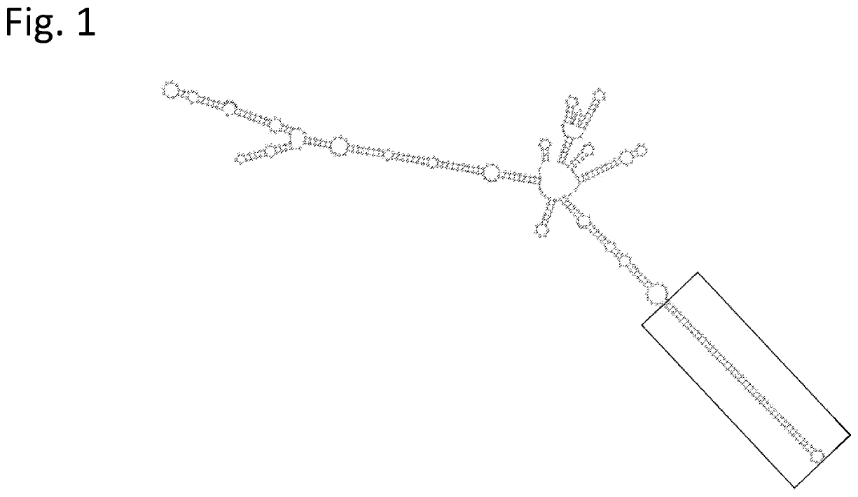

The resulting RNA vector, ELVd-hpRNA, (see FIG. 1; SEQ ID NO: 3), which targets tomato PDS (SIPDS, Gene ID: 544073), is transfected into tomato mesophyll protoplast cells, and its impact on PDS gene expression is detected with RT-qPCR. After the confirmation of PDS gene expression reduction by ELVd-hpRNA in protoplasts, the RNA vector is mechanically inoculated on tomato leaves by direct rubbing. Knockdown of PDS gene expression in tomato causes a photobleaching phenotype (Liu et al., The Plant Journal, 31(6): 777-786, 2002). The photobleaching phenotype is monitored for two weeks after inoculation, and the RNA vector and PDS gene expression are detected by RT-qPCR, as described herein.

### Characterization

The synthesized RNA vector, and ELVd alone as a control, are quantified using an Agilent 2100 Bioanalyzer system and analyzed using RNase H and RNase R assays, as described in Examples 6 and 7.

### Delivery and Bioassay

### 1. Protoplast Assay

Micro-Tom tomato (Totally Tomatoes) protoplast isolation is performed as described in Example 9 with the following modifications. The enzyme digestion step runs overnight (14 hours) at 26° C. with gentle shaking at 25 rpm. After overnight digestion, protoplast cells are collected and purified with sucrose-gradient centrifugation. The RNA vector, and ELVd as a control, are delivered as described in Example 9 to Micro-Tom tomato protoplasts with polyethylene glycol (PEG). The cells are harvested at 6 hours, 12 hours, and 24 hours after transfection.

RNA extraction is performed using the Maxwell® RSC simplyRNA Blood Kit (Promega; AS1380). Quantitative RT-PCR is employed on samples from different time points to measure transcript levels of the endogenous PDS gene and the RNA vector.

### 2. In Planta Assay

The RNA vector is mechanically inoculated into Micro-Tom tomatoes by leaf rubbing, as described in Example 10. Photobleaching phenotype is monitored for two weeks after inoculation. The inoculated leaves and distant leaves are imaged and processed using ImageJ to quantify photobleaching. RNA extraction is performed using the Maxwell RSC Plant RNA Kit (AS1500). Quantitative RT-PCR is employed on samples from different time points (12 hours, 1 day, 2 days, 4 days, 7 days, and 2 weeks following inoculation) to measure transcript levels of the endogenous PDS gene and the RNA vector, including siRNA produced downstream of hpRNA processing.

### Example 13. ELVd with a Guide RNA Targeting a Corn Gene

This example describes the modification of a viroid to produce and deliver an RNA molecule that includes an effector to a plant cell and edit the genome of the plant cell.

In this example, the RNA vector includes the following:
ELVd (Eggplant Latent Viroid) (SEQ ID NO: 1)
Effector: gRNA-g12: guide RNA (SEQ ID NO: 4) targeting a corn (Zea mays; Zm) endogenous gene, gl2 (glossy2)

The guide RNA targeting the corn gene glossy2 (Zmgl2) (SEQ ID NO: 6) is designed based on the LbCas12a gRNA1 provided in Lee et. al. (Plant Biotechnology Journal, 17(2): 362-372, 2019) with a AsCas12a direct repeat (DR, SEQ ID NO: 7) on both 5 and 3 ends. The guide RNA is inserted in U245-U246 of ELVd. The RNA vector (SEQ ID NO: 5) is synthesized in a bacterial viroid system, as described in Example 3.

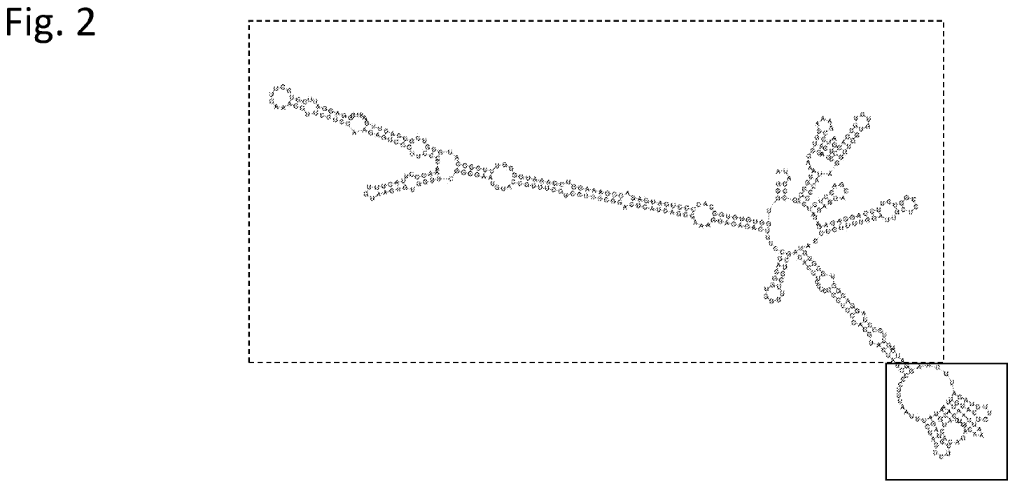

The resulting RNA vector, ELVd-gRNA, (see FIG. 2; SEQ ID NO: 5), which targets the corn g12 gene (Zmgl2, Gene ID: 103645956), is co-transfected with Acidaminococcus sp. Cas12a (AsCas12a; IDT™, Cat #10001272) into corn B73 mesophyll protoplast cells, and the editing efficiency is evaluated using Sanger sequencing and analyzed with the online tool ICE (Inference of CRISPR Edits) provided by Synthego.

### Characterization

The synthesized RNA vector, and ELVd alone as a control, are quantified using an Agilent 2100 Bioanalyzer system and analyzed using RNase H and RNase R assays, as described in Examples 6 and 7.

### Delivery and Bioassay

### 1. In Vitro AsCas12a Nuclease Cutting Assay

The synthesized RNA vector is incubated with AsCas12a in IDTE buffer (IDT™, Cat #11-05-01-05) for 30 minutes at room temperature or 37° C. and then analyzed by gel electrophoresis. The product after incubation is also analyzed by incubating with a PCR amplicon (1 kb) containing a gRNA-gl2 targeting site. The incubation is carried out at room temperature or 37° C. and then analyzed by gel electrophoresis.

### 2. Protoplast Assay

Maize B73 protoplast isolation is performed as described in Example 9. As a control, gRNA-gl2 is purchased from Integrated DNA Technologies (IDTT) as the standard AsCas12a crRNA. The gl2 crRNA is complexed with AsCas12a protein to form RNP following the manufacturer's protocol. RNP and the RNA vector are delivered as described in Example 9 to maize B73 protoplasts with polyethylene glycol (PEG). The cells are harvested at 24 hours after transfection.

Genomic DNA extraction is performed using the Maxwell RSC Plant DNA Kit (Cat #AS1490). A 1 kb PCR amplicon containing the guide RNA targeting region is amplified, followed by Sanger sequencing. The editing efficiency is calculated and analyzed with the online tool ICE (Inference of CRISPR Edits provided by Synthego.

In one experiment, an RNA vector was constructed to include a viroid sequence modified to include an effector, in this case a CRISPR guide RNA (gRNA) for editing a gene in a plant. More specifically, the vector included an Eggplant Latent Viroid (ELVd) modified to include the effector gRNA-LcPro3, a guide RNA targeting a corn (Zea mays; Zm) endogenous LC gene (see www[dot]maizegdb[dot]org/gene_center/gene/GRMZM5G822829). The guide RNA gRNA-LcPro3 used to target the corn gene, ZmLc (SEQ ID NO: 912) was designed based on the Cpf1 LcPro3 (SEQ ID NO: 913) disclosed in US Patent Application Publication US2019/0352655A1, with a AsCas12a direct repeat (“DR”, SEQ ID NO: 914) on both the 5′ and 3′ ends. This guide RNA sequence was inserted at U245-U246 of the native ELVd sequence. The resulting RNA vector, ELVd-gRNA (SEQ ID NO: 915) was synthesized using the in vitro transcription system described in Example 1. Circularization was carried out by incubating with T4 PNK (T4 Polynucleotide kinase, New England Biolabs, catalogue number M0201S) for 1.5 hours, followed by ligation with T4 ligase 1 (New England Biolabs, catalogue number) for 3 hours. The observed efficiency of ligation was 33.3%, calculated based on the corresponding band intensity in polyacrylamide gel electrophoresis. The circular ELVd-gRNA was enriched to about 63% following RNase R treatment (Lucigen, catalogue number RNR07250) and quantified using polyacrylamide gel electrophoresis and analyzed using RNase R assays, as described in Examples 7. The synthesized RNA vector ELVd-gRNA (SEQ ID NO: 915) was subjected to an in vitro AsCas12a nuclease cutting assay to verify the insertion of the guide RNA. Briefly, the vector was incubated with AsCas12a in IDTE buffer (IDT™, catalogue number 11-05-01-05) for 30 minutes at 37° C. and then analyzed by gel electrophoresis. A small band representing the guide RNA part (DR+LcPro3) was released from the full length ELVd-gRNA vector after AsCas12a incubation.

This vector, ELVd-gRNA (SEQ ID NO: 915) was tested for its ability to edit the target Lc gene in corn B73 mesophyll protoplast cells. Maize B73 protoplast isolation was performed as described in Example 9. As a control, gRNA-LcPro3 was purchased from Integrated DNA Technologies (IDT) as the standard AsCas12a crRNA. The LcPro3 crRNA was complexed with Acidaminococcus sp. Cas12a (AsCas12a; IDT, catalogue number 10001272) protein to form a ribonucleoprotein (RNP) following the manufacturer's protocol. The linear and circular ELVd-gRNA were either preassembled with AsCas12a to form RNPs or co-transfected with AsCas12a. These samples were delivered as described in Example 9 to maize B73 protoplasts with polyethylene glycol (PEG). The cells were harvested at 24 hours after transfection. Genomic DNA extraction was performed using Qiagen DNeasy Plant Mini Kit (Qiagen, catalogue number 69104). A 1107 bp PCR amplicon (SEQ ID NO: 916) containing the guide RNA targeting region was amplified with primers ZmLc-F (SEQ ID NO: 917) and ZmLc-R (SEQ ID NO: 918), followed by Sanger sequencing to determine the presence of edits. Editing efficiency was analyzed with the publicly available online tool TIDE (shinyapps[dot]datacurators[dot]nl/tide/). Results are provided in Table 9. The data demonstrate that viroids modified to include CRISPR guide RNAs are effective in editing a target gene.

TABLE 9
Results

| | IDT | IDT | Linear | Linear | Circular | Circular |
|---|---|---|---|---|---|---|
| | LcPro3 | LcPro3 | ELVd- | ELVd- | ELVd- | ELVd- |
| Replicate | crRNA* | crRNA** | gRNA* | gRNA** | gRNA* | gRNA** |
| 1 | 6.2 | 5.6 | 1.6 | 2.2 | 1.7 | 6.6 |
| 2 | 6.3 | 4.9 | 2.3 | 0.6 | 0.3 | 0.2 |
| 3 | 7.2 | 10.2 | 2.5 | 3 | 0.5 | 0.4 |

*transfected as a pre-assembled RNP
**co-transfected with AsCas12a

### Example 14. PSTVd with a Small RNA Targeting a Tomato Gene

This example describes the modification of a viroid to deliver an RNA molecule that includes an effector to a plant, thereby changing the phenotype of the plant.

In this example, the RNA vector includes the following:
PSTVd-RG1 (GenBank Acc. No. U23058): potato spindle tuber viroid strain RG1 (SEQ ID NO: 8)
Effector: sRNA-SIPDS: small RNA (SEQ ID NO: 9) targeting a tomato endogenous gene, PDS (phytoene desaturase)

The small RNA targeting SIPDS is derived from VIGS PDS RNAi design (Liu et al., The Plant Journal, 31(6): 777-786, 2002), with 21 nt targeting PDS. The region 191-211 nt in the sense strand (+) of PSTVd-RG1 is removed and replaced with the effector sRNA-SIPDS sequence. The RNA vector (SEQ ID NO: 10) is synthesized using in vitro transcription, as described in Example 2.

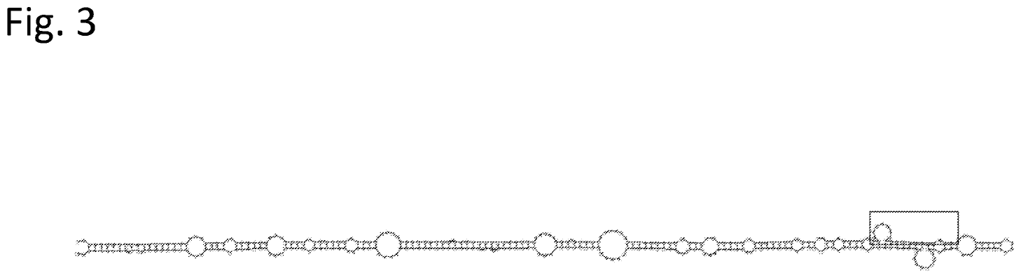

The resulting RNA vector, PSTVd-sRNA, (see FIG. 3; SEQ ID NO: 10), which targets the tomato gene PDS (SIPDS, Gene ID: 544073), is transfected into tomato mesophyll protoplast cells and its impact on PDS gene expression is detected with qRT-PCR. After the confirmation of PDS gene reduction by PSTVd-sRNA in protoplasts, the RNA vector is mechanically inoculated on tomato leaves by direct rubbing. Knockdown of PDS gene expression in tomato causes a photobleaching phenotype (Liu et al., The Plant Journal, 31(6): 777-786, 2002). The photobleaching phenotype is monitored for two weeks after inoculation, and the RNA vector and PDS gene expression are detected by qRT-PCR, as described herein.

### Characterization

The synthesized RNA vector, and PSTVd-RG1 alone as a control, are quantified using an Agilent 2100 Bioanalyzer system and analyzed using RNase H and RNase R assays, as described in Examples 6 and 7.

### Delivery and Bioassay

### 1. Protoplast Assay

Micro-Tom tomato protoplast isolation is performed as described in Example 12. The RNA vector and PSTVd-RG1 as control are delivered as described in Example 9 to Micro-Tom tomato protoplasts with polyethylene glycol (PEG). The cells are harvested at 6 hours, 12 hours, and 24 hours after transfection. RNA extraction is performed using the Maxwell® RSC simplyRNA Blood Kit (AS1380). Quantitative RT-PCR is employed on samples from different time points to measure transcript levels of the endogenous PDS gene and the RNA vector.

### 2. In Planta Assay

The RNA vector is mechanically inoculated to Micro-Tom tomato by leaf rubbing. The photobleaching phenotype is monitored for two weeks after inoculation. The infected leaves and distant leaves are imaged and processed using ImageJ to quantify. RNA extraction is performed using the Maxwell® RSC Plant RNA Kit (AS1500). Quantitative RT-PCR is employed on samples from different time points (12 hours, 1 day, 2 days, 4 days, 7 days, and 2 weeks following inoculation) to measure transcript levels of the endogenous PDS gene and the RNA vector.

In one experiment, a viroid was modified to deliver an RNA molecule including an effector to a plant, resulting in a change in the plant's phenotype. More specifically, this example illustrates a viroid vector based on potato spindle tuber viroid strain RG1 (SEQ ID NO: 8, PSTVd-RG1,GenBank Acc. No. U23058) and modified to include as the effector a small RNA, “sRNA-SIPDS” (SEQ ID NO: 9) that targets a tomato endogenous gene, PDS (phytoene desaturase). The sRNA-SIPDS sequence was derived from a viral-induced gene silencing (“VIGS”) PDS RNAi design described Liu et al. (2002) Plant J., 31:777-786, with 21 nucleotides that target PDS; suppression of PDS expression in tomato plants causes a photobleached phenotype. The region at positions 191-211 nt in the sense (+) strand of wild-type PSTVd-RG1 (SEQ ID NO:8) was deleted and replaced with the effector sRNA-SIPDS sequence (SEQ ID NO:9). The resulting RNA vector (SEQ ID NO:919) was synthesized using in vitro transcription, as described in Example 2. Two additional RNA vectors, “PSTVd-siRNA 1” (SEQ ID NO: 920) and “PSTVd-siRNA 2” (SEQ ID NO: 921), were constructed similarly to include the 21-nucleotide targeting the tomato PDS gene. The RNA vectors were individually inoculated into tomato leaves by rubbing (see Example 10) and the plants monitored for photobleaching for 7 weeks after inoculation.

The RNA vectors, PSTVd-sRNA, (SEQ ID NO: 919), “PSTVd-siRNA 1” (SEQ ID NO:920) and “PSTVd-siRNA 2” (SEQ ID NO: 921), which target the tomato gene PDS (SIPDS, Gene ID: 544073), and the wild-type PSTVd-RG1 as a control, were quantified using an Agilent 2100 Bioanalyzer system and analyzed using RNase H and RNase R assays, as described in Examples 6 and 7. The PSTVd-sRNA vector was mechanically inoculated on Rutgers tomato leaves by direct rubbing. Knockdown of PDS gene expression in tomato causes a photobleaching phenotype (Liu et al., The Plant Journal, 31(6): 777-786, 2002). The photobleaching phenotype was monitored for 7 weeks after inoculation, and the RNA vector and PDS gene expression were detected by qRT-PCR, as described herein. Leaf samples from the inoculated plants were taken, and RNA extracted using the MagMax™ mirVANA™ Total RNA Isolation Kit (A27828). Quantitative RT-PCR was used to measure transcript levels of the endogenous PDS gene and of the RNA vector, normalized to the reference gene GAPDH (GenBank ID: U93208.1). The percent suppression of the target gene PDS in PSTVd-sRNA-treated plants, relative to the PSTVd-RG1-treated control plants, is shown in Table 10.

TABLE 10
Results.

| | Percent Suppression: |
|---|---|
| Sample | Mean (SD) |
| PSTVd-sRNA (SEQ ID NO: 919) | 39.5% (27.4%) |
| PSTVd-siRNA 1 (SEQ ID NO: 920) | 34.9% (25.7%) |
| PSTVd-siRNA 2 (SEQ ID NO: 921) | 22.2% (49.7%) |

### Example 15. PSTVd TL-R or PSTVd R with a Pre-miRNA Targeting a Tomato Gene

This example describes the utilization of the replication motif from a viroid to replicate an RNA molecule that includes an effector in a plant cell or plant protoplast.

In this example, the RNA vector includes the following:
Replication domain of PSTVd (potato spindle tuber viroid), consisting of either:
   PSTVd TL-R (Table 2, SEQ ID NO: 11): the left-terminal region of PSTVd, including a binding site for the DNA-dependent RNA polymerase II (Pol II) and the transcription factor IIIA containing seven zinc finger domains (TFIIIA-7ZF); or
   PSTVd TL-CCR (SEQ ID NO: 12): the left-terminal and central conserved region of PSTVd
Effector: amiR-PDS: a pre-miRNA (SEQ ID NO: 13) targeting a plant endogenous gene, PDS (phytoene desaturase)

Pre-miRNA targeting tomato (Solanum lycopersicum; SI) PDS (SIPDS) (amiR-SIPDS, SEQ ID NO: 13) is designed with Web MicroRNA Designer (WMD3) on the Arabidopsis precursor MIR319a. The amiR-SIPDS is fused to PSTVd TL-R (SEQ ID NO: 11) or PSTVd TL-CCR (SEQ ID NO: 12). The RNA vectors (SEQ ID NO: 14 and SEQ ID NO: 15) are synthesized with in vitro transcription, as described in Example 2. Two forms (linear and circular) of each RNA vector are synthesized and tested in protoplast cell assays.

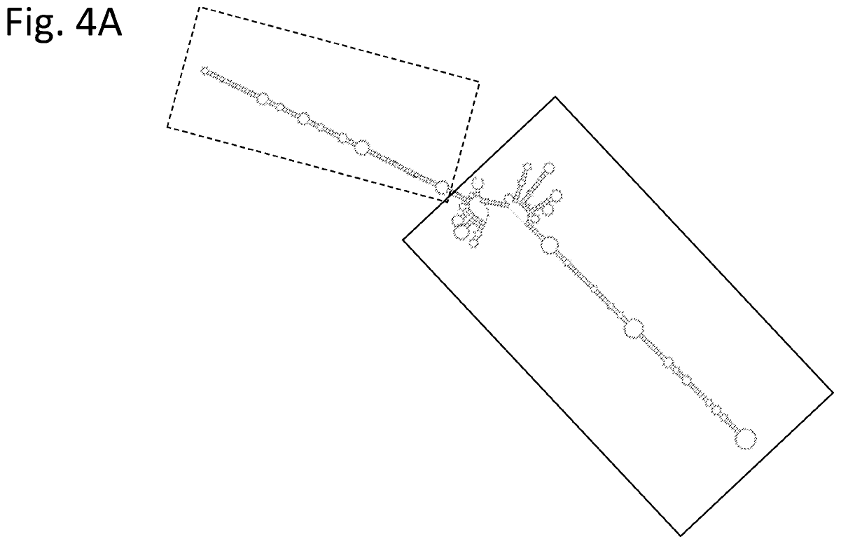

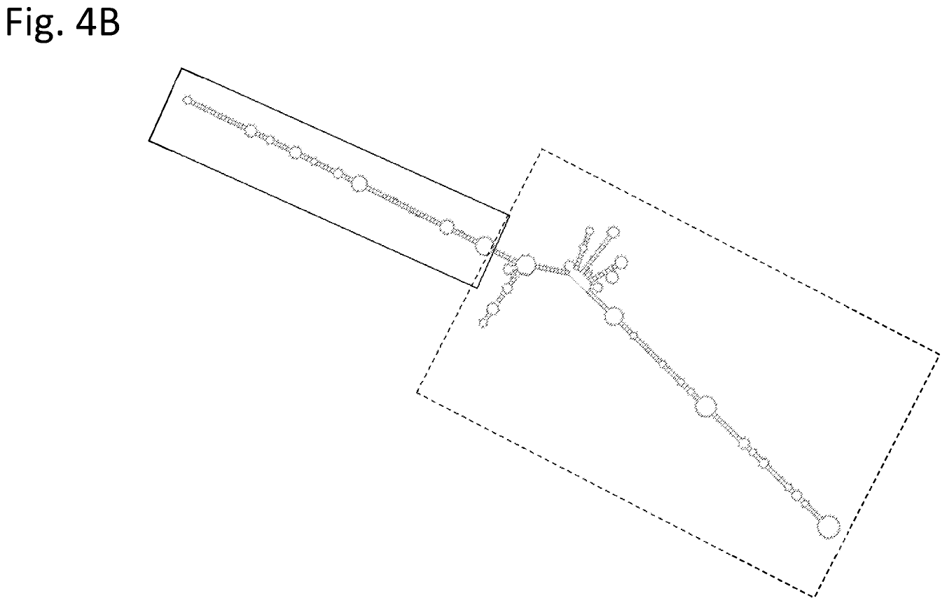

The resulting RNA vectors, TL-R-amiR-PDS (FIG. 4A; SEQ ID NO: 14) and R-amiR-PDS (FIG. 4B; SEQ ID NO: 15), which target tomato PDS (SIPDS, Gene ID: 544073), are transfected into tomato mesophyll protoplast cells. Replication of the RNA vectors and their impact on PDS gene expression are detected using qRT-PCR.

### Characterization

The synthesized linear and circular fusion RNA vectors are quantified using an Agilent 2100 Bioanalyzer system and analyzed using RNase H and RNase R assays, as described in Examples 6 and 7.

### Delivery and Bioassay

The RNA vectors are delivered to Micro-Tom tomato protoplasts with polyethylene glycol (PEG), and their replication is detected by qRT-PCR measurement of RNA vectors and of transcripts of the endogenous PDS gene.

Micro-Tom tomato protoplast isolation is performed as described in Example 12. The RNA vector and PSTVd-RG1 as control are delivered as described in Example 9 to Micro-Tom tomato protoplasts with polyethylene glycol (PEG). The cells are harvested at 6 hours, 12 hours, and 24 hours after transfection. RNA extraction is performed using the Maxwell® RSC simplyRNA Blood Kit (AS1380). Quantitative RT-PCR is employed on samples from different time points to measure transcript levels of the endogenous PDS gene and the circular and linear fusion RNAs.

### Example 16. Arabidopsis circRNA with a PSTVd Replication Motif

This example describes the use of an RNA replication motif to amplify the effects of an endogenous regulatory RNA in a plant.

In this example, the RNA vector includes the following:
PSTVd Left Terminal domain (TL; SEQ ID NO: 33)
Effector: circGORK: circular RNA (SEQ ID NO: 16) derived from the intron segment flanking exons 2 and 3 of the Arabidopsis GATED OUTWARDLY-RECTIFYING K+ CHANNEL (GORK) gene. GORK regulates stomatal opening and drought resistance.

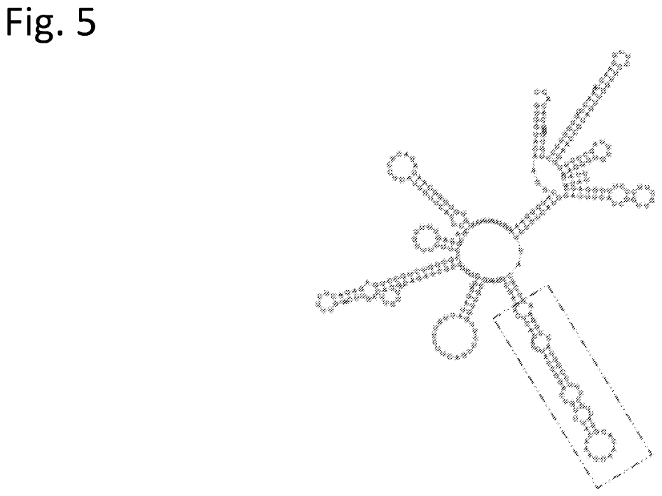

The PSTVd TL region is as shown in Table 2. The circGORK is designed according to the endogenous circular RNA detected and described by Zhang et al., The Plant Journal, 98(4): 697-713, 2019. The RNA vector (SEQ ID NO: 17) is synthesized using the in vitro transcription system described in Example 2. The resulting RNA vector, PSTVd/TL-circGORK, (see FIG. 5; SEQ ID NO: 17), which targets drought resistance pathways, is transfected into Arabidopsis plants by mechanical leaf rubbing, as described herein, and its ability to replicate in leaves is quantified using qRT-PCR. Once presence and replication of RNA is confirmed, plants are subjected to a drought assay to measure the ability of PSTVd/TLR-circGORK to induce a drought resistance phenotype as described by Zhang et al., The Plant Journal, 98(4): 697-713, 2019 for circGORK.

### Characterization

The synthesized RNA vector (SEQ ID NO: 17) and PSTVd alone as a control are quantified using an Agilent 2100 Bioanalyzer system and analyzed using RNase H and RNase R assays, as described in Examples 6 and 7.

### Delivery and Bioassay

The RNA vector is mechanically inoculated to Arabidopsis by leaf rubbing. Control and inoculated plants are subjected to a drought stress as described by Zhang et al., The Plant Journal, 98(4): 697-713, 2019. Briefly, the soil moisture of each treatment is monitored by measuring the relative soil water content, followed by rationing of water to maintain a designated soil moisture. The pots are weighed and watered twice daily. After the most serious stress reached the preset levels, the plants are maintained for an extra 1 day, then the whole plants are harvested for fresh weight measurement and leaves are harvested for RNA extraction. Fresh weight of drought-resistant plants is higher than that of control plants.

RNA extraction is performed using the Maxwell RSC simplyRNA Blood Kit (AS1380). Quantitative RT-PCR is employed on samples from different time points to measure transcript levels of the RNA vector.

### Example 17. In Planta Viral Trafficking Motifs Conjugated to a Fluorescent Aptamer as a Circular Fusion RNA

This example demonstrates the in planta trafficking of a circular fusion RNA conjugated to a fluorescent aptamer using splint ligation. The circular fusion RNAs contain a PSTVd loop 27 (Table 2) viral motif sequence (5′-UUUUCA-3′; SEQ ID NO: 18) previously described to be essential for viral trafficking within the host plant (V W et al., PLoS Pathogens, 15(10): e1008147, 2019). The fusion RNA is synthesized as described in Example 2. This trafficking motif is synthesized in tandem with either an intact or split Broccoli RNA aptamer sequence as an exemplary cargo.

### Design

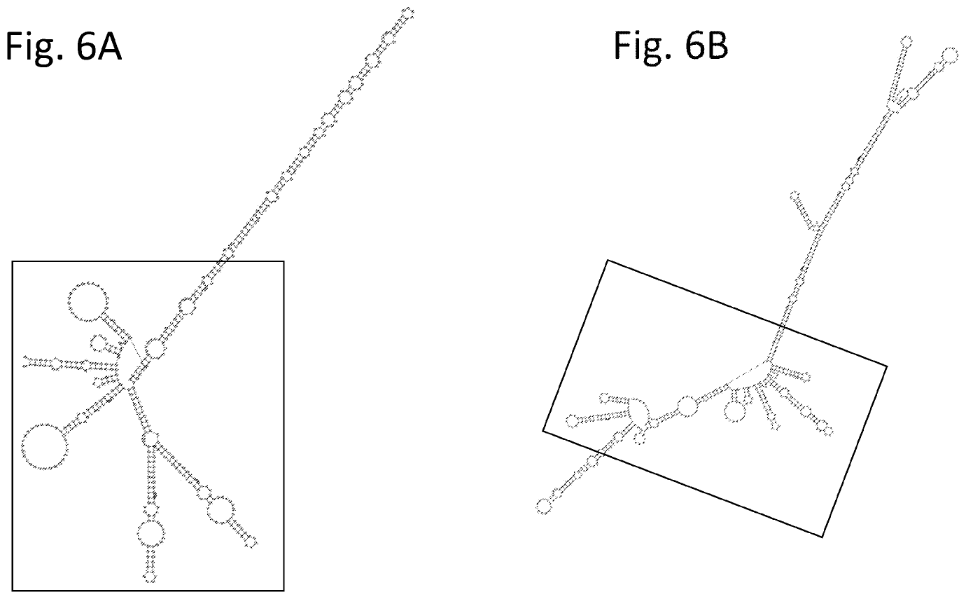

A circular fusion RNA 1 (CircRNA1) construct (FIG. 6A; SEQ ID NO: 20) is generated containing the following elements:
PSTVd right terminal domain containing transmission motifs (loop 26 and loop 27) (SEQ ID NO: 18)
Intact Broccoli RNA aptamer sequence (SEQ ID NO: 19)

A circular fusion RNA 2 (CircRNA2) construct (FIG. 6B; SEQ ID NO: 23) is generated containing the following elements:
PSTVd right terminal domain containing transmission motifs (loop 26 and loop 27) (SEQ ID NO: 18)
Linker region (SEQ ID NO: 21)
Split Broccoli aptamer sequence (SEQ ID NO: 22)

Linear transcripts are synthesized using a T7 in vitro transcription reaction, and circular fusion RNAs are generated using T4 RNA ligase 2 on a 20 nt splint DNA oligomer template (SEQ ID NO: 24), as described in Example 2.

### Characterization

The ratio of linear to circular fusion RNAs is detected after incubation with RNase R and migration of samples on a 6% PAGE gel. Transcript topology can also be visualized by circularization-dependent aptamer coordination of the fluorescent molecule DFHBI-1T.

### Delivery and Bioassay

To test whether the viral motif embodied in the circRNA is capable of trafficking throughout the tomato plant, the synthesized, circularized CircRNA1 or CircRNA2 is rubbed onto a leaf at the base of an Arabidopsis thaliana plant. Linear transcripts of CircRNA1 and CircRNA2 are provided as negative controls. Leaves distal to the site of inoculation are analyzed for Broccoli aptamer transcripts by qRT-PCR. Successful trafficking of the circular RNA throughout the plant is confirmed by distal plant structures containing circRNA transcripts, as measured by qRT-PCR. Transcripts in distal leaves in circular RNA are not expected to be observed in plants treated with constructs that do not have the trafficking motif sequence. Additionally, efficiently circularized and trafficked RNA constructs are visualized by green fluorescence in distal leaf structures when incubated with 10 μM DFHBI-1T fluorogen (Tocris, 5610) for 30 minutes to one hour. Linear constructs are expected to have lower or absent fluorescence when compared with CircularRNAs. Linear CircRNA1 containing the intact Broccoli aptamer is expected to have lower fluorescence than a circularized CircRNA1. Linear CicRNA2 containing the split Broccoli aptamer is expected to have no fluorescence and will only fluoresce upon circularization and formation of the full aptamer sequence.

Detection of Broccoli aptamer transcripts, as well as the emission of green fluorescence at 472 nm, indicates both efficient circularization and trafficking of the circular fusion RNA.

### Example 18. PSTVd with a Spinach RNA Aptamer in Arabidopsis and Corn

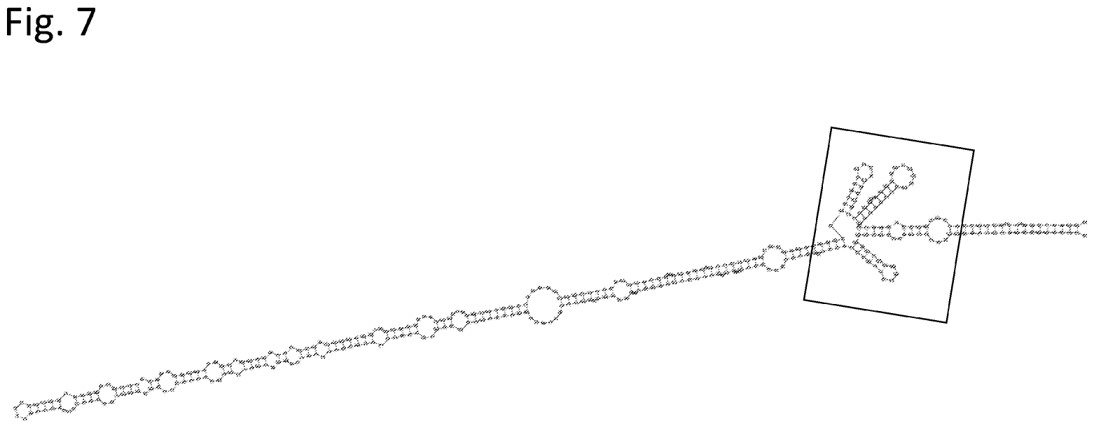

This example describes PSTVd infection that is host-specific. To illustrate host specific infection of PSTVd, the pathogenicity domain (SEQ ID NO: 25) of PSTVd is deleted and replaced with a Spinach RNA aptamer (SEQ ID NO: 26; FIG. 7). The RNA construct further contains a linker (1-100 nt) containing one or more of randomly generated sequence; contiguous or split fluorescent aptamers (e.g., Baby_spinach, Mango3, or Broccoli); and sequence derived from non-coding plant RNAs retrieved from publicly available RNAseq databases. In vitro transcription of RNAs is performed using a T7 in vitro transcription reaction.

### Design

In this example, the RNA vector includes the following:
PSTVd lacking the pathogenicity domain (See Table 2)
Spinach aptamer (SEQ ID NO: 26)

### Characterization

RNAs are treated with DNase to remove the DNA template. Linear RNAs are then column-purified using the Zymo RNA Clean & Concentrator-5 kit (Zymo Research: R1014). Linear transcribed RNAs are quality tested by heating in vitro transcription products to 80° C. for 7-10 minutes. Heated RNAs are then run on a 6% denaturing PAGE gel to validate purity of transcribed RNA and RNA quality.

### Delivery and Bioassay

The linear PSTVd-Spinach fusion RNA (SEQ ID NO: 27) is rubbed onto the leaves of Arabidopsis and corn plants to test for infection of the host (Arabidopsis) and non-host species (corn). Leaves distal and proximal to the site of inoculation are removed and incubated with 10 μM DFHBI-1T fluorogen (Tocris, 5610) for 30 minutes to one hour. Successful circularization and infection of the host plant results in an emission of green fluorescence at 472 nm. Spinach RNA aptamer transcripts are also quantified using qRT-PCR of proximal and distal leaves after inoculation. Absence of PSTVd-Spinach transcripts in distal corn leaves will indicate that PSTVd infection is specific to previously described host plants.

### Example 19. An RNA Motif to Traffic Aptamers to a Subcellular Location

This example describes the use of an RNA targeting motif to traffic a Spinach RNA aptamer to a subcellular location, the chloroplast.

In this example, the RNA vector includes the following:
ELVd (Eggplant Latent Viroid) (SEQ ID NO: 1) Effector: Spinach RNA aptamer (SEQ ID NO: 26) ELVd is designed using the sequence of the complete eggplant latent viroid genome (GenBank Acc. No. AJ536613.1; SEQ ID NO: 28) and the repetition of the plus-strand hammerhead ribozyme domain for the longer-than-unit ELVd (SEQ ID NO: 1); see, e.g., Branch et al. (1981) Proc. Natl. Aced. Sci. USA, 78:6381-6381, DOI: 10.1073/pnas.78.10.6381; Tabler et al. (1992) Virology, 190:746-753, DOI: 10.1016/0042-6822(92)90912-9; Cordero et al. (2018) Frontiers Microbiol., 9:635, DOI: 10.3389/fmicb.2018.00635; Flores et al. (2012) Frontiers Microbiol., 3:217, DOI: doi.org/10.3389/fmicb.2012.00217. The Spinach RNA aptamer is designed using the sequence described in Example 3. The RNA vector (SEQ ID NO: 29) is synthesized using a bacterial transcription system as described in Example 3.

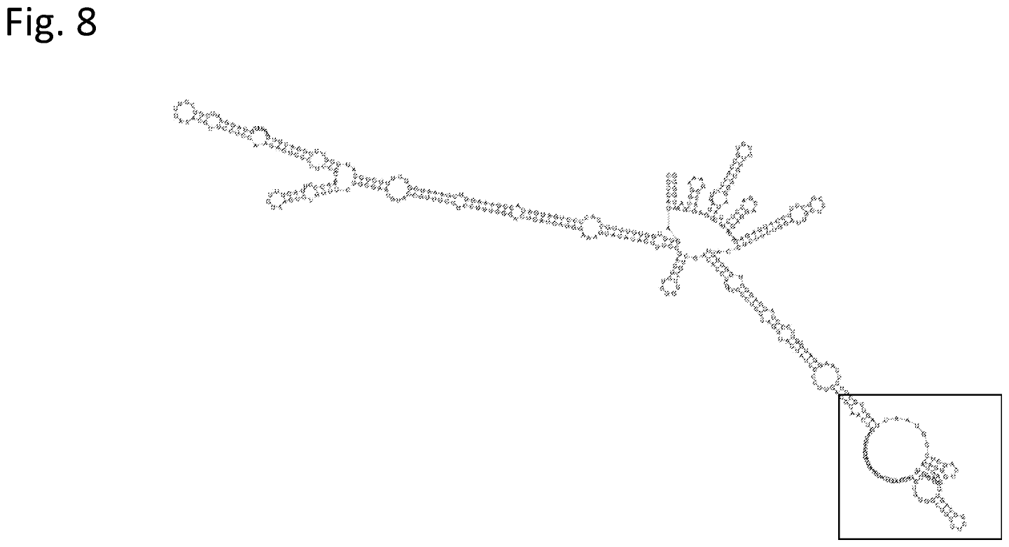

The resulting RNA vector, ELVd-Spinach, (see FIG. 8B; SEQ ID NO: 29) is transfected into tomato mesophyll protoplast cells and its trafficking to chloroplasts is detected with qRT-PCR and fluorescence imaging. After the confirmation of trafficking, the RNA vector is mechanically inoculated on tomato leaves by direct rubbing. Fluorescence and RNA presence are quantified in leaves by imaging and qRT-PCR.

### Characterization

The synthesized RNA vector (SEQ ID NO: 29) and linear RNA controls are quantified using an Agilent 2100 Bioanalyzer system and analyzed using RNase H and RNase R assays, as described in Examples 6 and 7.

### Delivery and Bioassay

### 1. Protoplast Assay

Micro-Tom tomato protoplast isolation is performed as described in Example 9 with modifications listed here. Micro-Tom tomato protoplast isolation is performed as described in Example 12. The RNA vector and linear RNA as control are delivered as described in Example 9 to Micro-Tom tomato protoplasts with polyethylene glycol (PEG). The cells are harvested at 6 hours, 12 hours, and 24 hours after transfection. Quantitative RT-PCR is employed on samples from each time point to measure transcript level of the RNA vector.

### 2. In Planta Assay

The RNA vector is mechanically inoculated into Micro-Tom tomatoes by leaf rubbing, as described in Example 10. Quantitative RT-PCR is employed on samples from different time points (12 hours, 1 day, 2 days, 4 days, 7 days, and 2 weeks following inoculation) to measure transcript levels of the RNA vector. Spinach RNA aptamer fluorescence is detected using the protocol described in Example 3 using leaf tissue.

### Example 20. An RNA Motif to Traffic Pre-miRNA to a Specific Cell Type

This example describes the use of an RNA targeting motif to traffic and deliver an RNA molecule that includes an effector to a specific plant cell type and change its phenotype.

In this example, the RNA vector includes the following:
PSTVd right terminal domain containing transmission motifs (loop 26 and loop 27) (PSTVd TR; SEQ ID NO: 18).
Effector tomato PDS (phytoene desaturase) pre-miRNA (amiR-PDS) (SEQ ID NO: 13).

Loop 27 (L27) (177 to 182 nts) (SEQ ID NO: 18) of PSTVd is selected based on the sequence identified by V W et al., PloS Pathogens, 15(10): e1008147, 2019. This RNA motif enables trafficking of the vector from epidermal to palisade spongy mesophyll cells. The RNA vector is synthesized using in vitro transcription and splint ligation, as described in Example 2.

The resulting RNA vector (SEQ ID NO: 32), PSTVd/TR-amiRPDS (FIG. 9), which targets tomato PDS (SIPDS, Gene ID: 544073), is transfected into tomato mesophyll protoplast cells, and its impact on PDS gene expression is detected with qRT-PCR. After the confirmation of PDS gene reduction by PSTVd/TR-amiRPDS in protoplasts, the RNA vector is mechanically inoculated on tomato leaves by direct rubbing. Knockdown of PDS gene expression in tomato causes a photobleaching phenotype (Liu et al., The Plant Journal, 31(6): 777-786, 2002). The photobleaching phenotype is monitored for two weeks after inoculation, and the RNA vector and PDS gene expression are detected by qRT-PCR, as described herein.

### Characterization

The synthesized RNA vector (SEQ ID NO: 32) and linear RNA controls are quantified using an Agilent 2100 Bioanalyzer system and analyzed using RNase H and RNase R assays, as described in Examples 6 and 7.

### Delivery and Bioassay

### 1. In Planta Assay

The RNA vector is mechanically inoculated into Micro-Tom tomatoes by leaf rubbing, as described in Example 10. Photobleaching phenotype is monitored for two weeks after inoculation. The inoculated leaves and distant leaves are imaged and processed using ImageJ to quantify photobleaching. RNA extraction is performed using the Maxwell® RSC Plant RNA Kit (AS1500). Quantitative RT-PCR is employed on samples from different time points (12 hours, 1 day, 2 days, 4 days, 7 days, and 2 weeks following inoculation) to measure transcript levels of the endogenous PDS gene and the RNA vector, including miRNA produced downstream of pre-RNA processing.

### Example 21: An RNA Motif to Traffic an Aptamer to a Specific Tissue Type

This example describes the use of an RNA targeting motif to traffic and deliver an RNA molecule that includes an aptamer to a specific plant tissue type.

In this example, the RNA vector includes the following:
PSTVd Left Terminal domain (TL; SEQ ID NO: 33)
Effector: Spinach RNA aptamer SEQ ID NO: 26

Loop 6 (L6) (U43/C318) (SEQ ID NO: 33) of PSTVd is selected based on the motif identified by Zhong et al., The EMBO Journal, 26(16): 3836-3846, 2007. This RNA motif enables trafficking of the vector into vascular tissue for transport into non-inoculated parts of the plant. The RNA vector is synthesized using in vitro transcription and splint ligation, as described in Example 2.

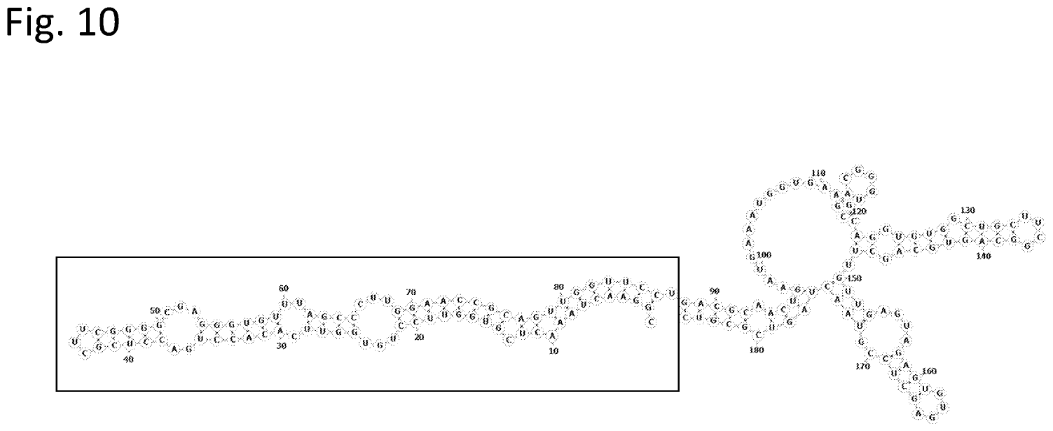

The resulting RNA vector, PSTVd/TL-Spinach, (SEQ ID NO: 34; FIG. 10) is mechanically inoculated on tomato leaves by direct rubbing. Fluorescence and RNA presence are quantified in distal tissues by imaging and qRT-PCR.

### Characterization

The synthesized RNA vector (SEQ ID NO: 34) and linear RNA controls are quantified using an Agilent 2100 Bioanalyzer system and analyzed using RNase H and RNase R assays, as described in Examples 6 and 7.

### Delivery and Bioassay

### 1. In Planta Assay

The RNA vector is mechanically inoculated to Micro-Tom tomato by leaf rubbing, as described in Example 10. This delivers the vector to epidermal cells, and trafficking to vascular tissue is enabled by Loop 6. Quantitative RT-PCR is employed on inoculated and non-inoculated leaf, stem and root tissue samples from different time points (12 hours, 1 day, 2 days, 4 days, 7 days, and 2 weeks following inoculation) to measure transcript levels of the RNA vector. Spinach RNA aptamer fluorescence is detected using the protocol described in Example 3 using all tissues.

### Example 22. Circular RNAs with PSTVd LT and EMCV or Maize HSP101 IRES in Monocots Vs. Dicots

This example demonstrates in vitro synthesis of circular fusion RNA and the subsequent expression of circRNA in planta.

### Design

In this example, the RNA vector includes the following:

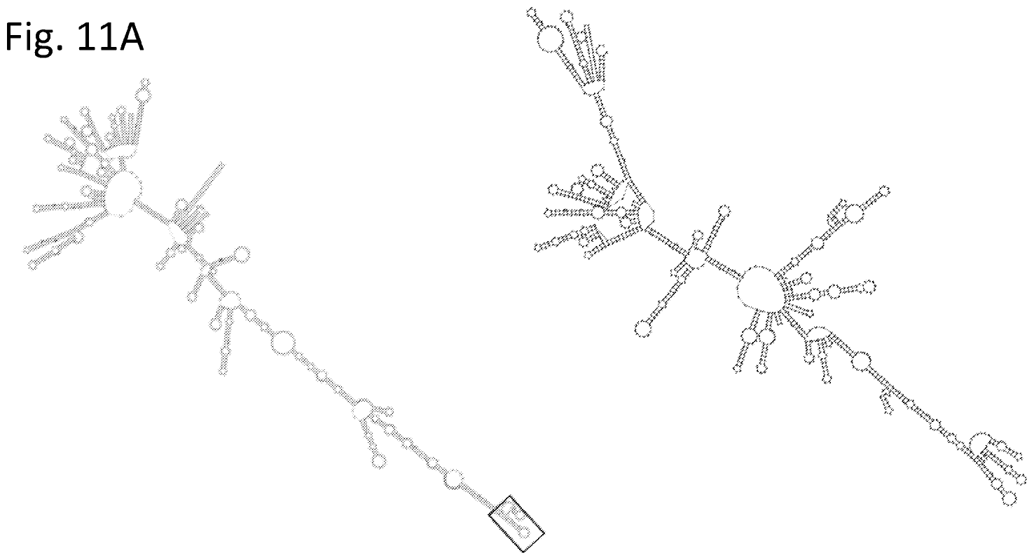

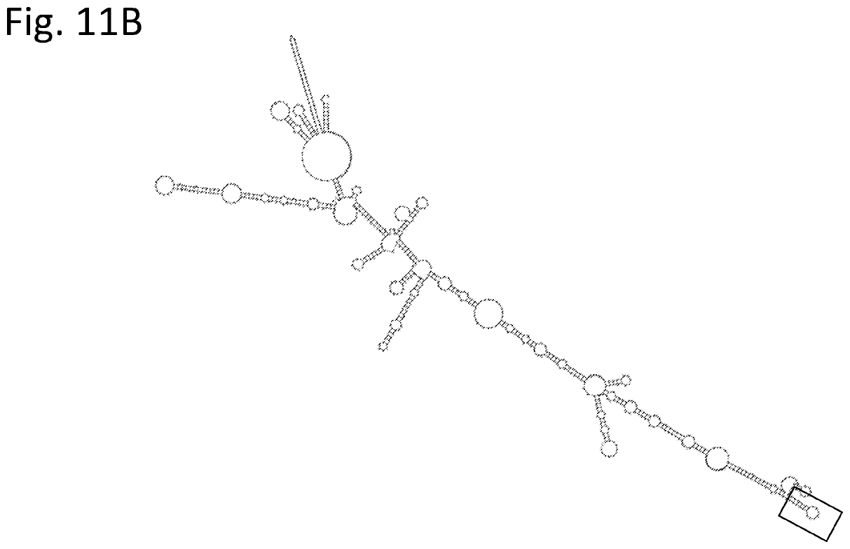

A first vector (FIG. 11A)
PSTVd left terminal region (PSTVD LT) (SEQ ID NO: 18)
Encephalomyocarditis virus internal ribosome entry site (EMCV IRES) (SEQ ID NO: 37)
PSP72 cloning vector (SEQ ID NO: 49)
Nanoluciferase (SEQ ID NO: 39)

A second vector (FIG. 11B)
PSTVd left terminal region (PSTVD LT) (SEQ ID NO: 18)
Maize HSP101 internal ribosome entry site (MAIZE HSP101 IRES) (SEQ ID NO: 38)
PSP72 cloning vector (SEQ ID NO: 49)
Nanoluciferase (SEQ ID NO: 39)

A circular RNA construct (FIGS. 11A and 11B, SEQ ID NO: 35 or SEQ ID NO: 40) is generated and contains the left terminal region of the PSTVd viroid sequence (SEQ ID NO: 36) found in Table 1 linked to an encephalomyocarditis virus (EMCV) internal ribosomal entry site (IRES) (SEQ ID NO: 37) or a maize heat shock protein of 101 KDa (HSP101) IRES (SEQ ID NO: 38) found on the IRESite online database for IRES sequences. IRES elements are placed upstream of the protein coding luciferase enzyme (SEQ ID NO: 39) and is subcloned into a pSP72 expression vector (Promega, REF: P2221). In vitro transcription of RNAs is performed using the T7 RNA polymerase promoter. Circular RNA precursors are synthesized using the HiScribe™ T7 Quick High Yield RNA Synthesis Kit (New England BioLabs® Inc., REF: E2050S). DNase is incubated with the in vitro transcription products to remove template DNA. CircularRNA is enriched as previously described in Example 16 by starting with 20 μg of RNA diluted in water. RNAs are then heated to 65° C. for 3 minutes, followed by incubating RNAs with RNase R at 37° C. for 15 minutes. Splint ligation is performed as described in Example 2.

### Characterization

Circular RNA is column purified using MEGAClear™ Transcription Clean-Up Kit (ThermoFisher, AM1908) as previously described (Wesselhoeft et al., Nature Communications, 9: Article no. 2629, 2018) and in Example 8.

### Delivery and Bioassay

Plant protoplasts are isolated as described in Example 9 and are incubated with linear or circular RNAs for 24 hours. Circular RNAs containing the EMCV IRES-luciferase or maize HSP101 IRES-luciferase are transfected into Arabidopsis and maize protoplasts respectively.

To measure the total level of transcribed RNAs of luciferase or fluorescent proteins, quantitative reverse transcription PCR (qRT-PCR) is performed. The total RNA as well as RNA treated with RNase R to enrich for circularRNA is converted to cDNA using cDNA Superscript™ III First Strand Synthesis System with random hexamers according to the manufacturer's instructions (ThermoFisher Scientific, 18080051). Additionally, the transcription efficiency of the IRES sequence in the circular RNA construct is measured using RNA scope in situ hybridization to quantify the total number of RNA transcripts as well as the identity and spatial localization of RNA transcripts within plant structures (ACD Bio CAT NO: 323120). Additionally, luciferase expression is quantified using the NanoLuc® kit (Promega) using a Spectramax® i3x Multi-Mode Plate Reader (Molecular Devices).

Additionally, 10⁵ plant protoplasts containing circular RNAs are homogenized and collected in Lysis Buffer (Promega, A8261). Protein lysates are collected and antibodies against GFP or luciferase are used to demonstrate protein expression relative to plant protoplasts that do not contain circular RNAs. Proteins are separated on a SDS-PAGE gel (BioRad). A commercially available standard (BioRad) is used as a size marker. After samples are electrotransferred to a polyvinylidene fluoride (PVDF) membrane (BioRad, 1704156), transfers are performed using a Trans-Blot® Turbo™ Transfer System (BioRad, 1704150) according to the manufacturer's protocol and visualized on a chemiluminescent kit (Rockland, KCA001). It is expected that strong luciferase expression in the linear RNA negative control will be absent due to degradation of the linear transcript by endogenous exonucleases in vitro, compared with strong expression of luciferase produced from circular RNAs.

### Example 23. PSTVd Trafficking Loop, Replication Loop, and Spinach RNA Aptamer

This example describes the generation of a circular fusion RNA capable of replicating and trafficking through a host plant. In this example, the PSTVd left terminal domain comprising loops 1-6 (SEQ ID: 41), fused to Spinach RNA aptamer (SEQ ID: 26), the PSTVD right terminal region containing the trafficking loop 27 (SEQ ID: 18), and the PSTVd left terminal domain containing the RNA pol II and TFIIIA-7ZF domains required for replication (SEQ ID NO: 42) (Jiang et al., Journal of Virology, 92(20): e1004-18, 2018) are fused to the Spinach RNA aptamer (SEQ ID NO: 26) and PSTVd trafficking loop 27 (SEQ ID NO: 42) respectively.

### Design

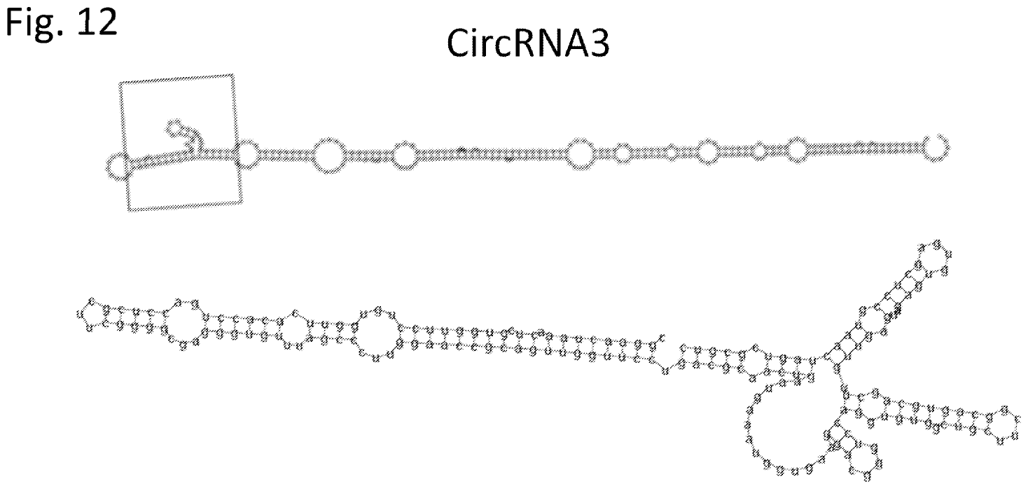

A circular fusion RNA 3 (CircRNA3; FIG. 12; SEQ ID NO: 43) construct is generated containing the following elements:
PSTVd viral trafficking motif sequence (SEQ ID NO:42)
Spinach RNA aptamer (SEQ ID NO: 26)
PSTVd replication motif sequence (SEQ ID NO: 41)

### Characterization

RNAs are treated with DNase to remove the DNA template. Linear RNAs are then column purified using the Zymo RNA Clean & Concentrator-5 kit (Zymo Research: R1014). To confirm the purity and quality of transcribed RNAs, an aliquot of RNA is heated to 80° C. for 10 minutes and run on a 6% denaturing PAGE gel as described in Example 16.

### Delivery and Bioassay

To test whether the viral motif embodied in the circular RNA is capable of replicating and trafficking throughout the tomato plant, the synthesized, circularized fusion RNA is rubbed onto a leaf at the base of an Arabidopsis thaliana plant (as described in Example 10). Leaves and stems that are distal to the site of inoculation are analyzed for Spinach aptamer transcripts by qRT-PCR. Linear constructs are expected to have lower or absent fluorescence when compared with circular RNAs. Linear CircRNA3 containing the intact Spinach aptamer is expected to have lower fluorescence than a circularized CircRNA 3.

Additionally, efficiently circularized and trafficked and replicated RNA constructs can be visualized by green fluorescence in distal leaf and stem structures when incubated with the 10 μM DFHBI-1T fluorogen (Tocris, 5610) for 30 minutes to one hour. Detection of Spinach aptamer transcripts, as well as the emission of green fluorescence at 472 nm, indicate both efficient circularization and trafficking of the circular fusion RNA.

### Example 24. An RNA Vector Containing Two RNA Motifs from Two Different Viroid Sources to Deliver an Aptamer to a Specific Cell Type

This example describes the use of a first RNA motif to replicate and amplify the vector, and a second RNA targeting motif to traffic and deliver an RNA molecule that includes an aptamer to a specific plant cell type.

In this example, the RNA vector includes the following:
PSTVd Left Terminal domain (TL; SEQ ID NO: 33)
Terminal left region (TLR) (1 to 72 nts) of tomato planta macho viroid (TPMVd) isolate Mex8 (SEQ ID NO: 45) (GenBankAcc. No. GQ131573.1)
Effector Spinach RNA aptamer (SEQ ID NO: 26)

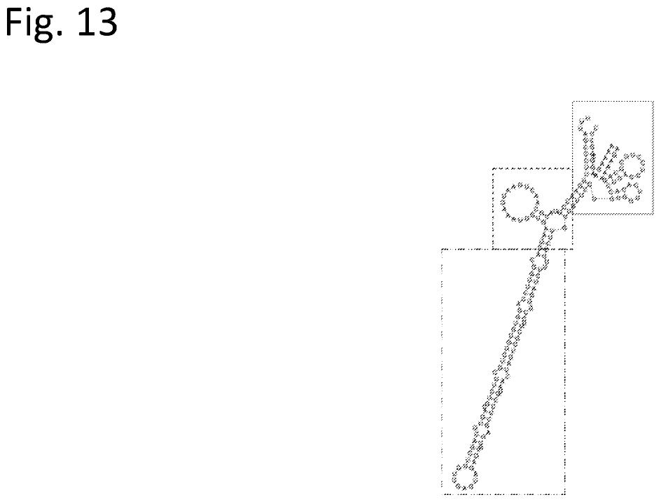

The resulting RNA vector, PSTVd/TL-Spinach-TPMVd/TLR, (see FIG. 13; SEQ ID NO: 46) is mechanically inoculated on tomato leaves by direct rubbing. Fluorescence and RNA presence are quantified in distal tissues by imaging and qRT-PCR.

### Design

The TL is selected according to the region identified by Wang et al., Plant Cell, 28: 1094-1107, 2016. This RNA motif enables replication of the vector in host cells. The TLR (1 to 72 nts) of TPMVd isolate Mex8 (SEQ ID NO: 45) (GenBank Acc. No. GQ131573.1) is designed according to the region identified by Yanagisawa et al., Virology, 526: 22-31, 2019. This second RNA motif enables trafficking of the RNA vector to pollen. The RNA vector is synthesized using in vitro transcription and splint ligation, as described in Example 2.

### Characterization

The synthesized RNA vector and linear RNA controls are quantified using an Agilent 2100 Bioanalyzer system and analyzed using RNase H and RNase R assays, as described in Examples 6 and 7.

### Delivery and in Planta Bioassay

The RNA vector is mechanically inoculated to Micro-Tom tomato by leaf rubbing, as described in Example 10. Quantitative RT-PCR is employed on inoculated leaf and pollen grain samples from different time points (12 hours, 1 day, 2 days, 4 days, 7 days, and 2 weeks following inoculation) to measure transcript levels of the RNA vector. Spinach RNA aptamer fluorescence is detected using the protocol described in Example 3 using leaf and pollen tissues.

### Example 25. In Planta Circularization of Viral Trafficking Motifs Conjugated to a Fluorescent Aptamer Through Transfection of a Linear Fusion RNA

This example demonstrates the in planta introduction of a linear fusion RNA and detection of subsequent endogenous circularization by detection of a fluorescent aptamer. The circular fusion RNAs contain a PSTVd loop 27 viral motif sequence (5′-UUUUCA-3′) (SEQ ID NO: 18) previously described to be essential for viral trafficking within the host plant, synthesized as described in Example 16 (V W et al., PloS Pathogens, 15(10): e1008147, 2019). This trafficking motif is synthesized in tandem with an intact Broccoli RNA aptamer as described in Example 16 (SEQ ID NO: 19).

### Design

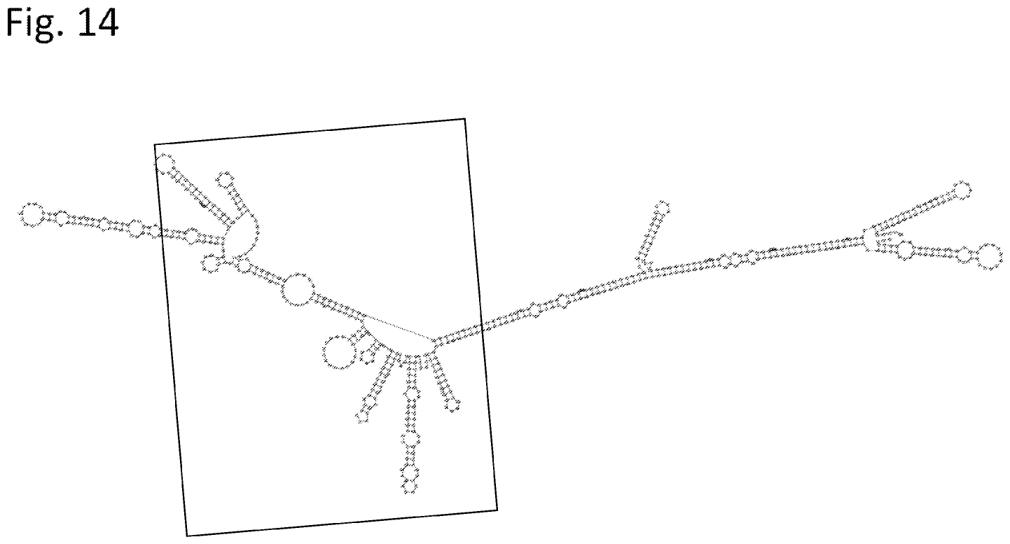

A circular fusion RNA 4 (CircRNA4; FIG. 14; SEQ ID NO: 47) construct is generated containing the following elements:
PSTVd viral trafficking motif sequence (SEQ ID NO: 18)
Intact Broccoli RNA aptamer sequence (SEQ ID NO: 19)

### Characterization

RNAs are treated with DNase to remove the DNA template. Linear RNAs are then column purified using the Zymo RNA Clean & Concentrator-5 kit (Zymo Research: R1014). To confirm the purity and quality of transcribed RNAs, an aliquot of RNA is heated to 80° C. for 10 minutes and run on a 6% denaturing PAGE gel as described in Example 16.

### Delivery and Bioassay

To test whether a linearized circular fusion RNA is capable of endogenous circularization after inoculation and trafficking throughout the tomato or Arabidopsis thaliana plant, linearized CircRNA1 (as previously described in Example 16) is rubbed onto a leaf at the base of the tomato or Arabidopsis thaliana plant. Proximal and distal leaves are then removed and incubated with 10 μM DFHBI-1T fluorogen (Tocris, 5610) for 30 minutes to one hour. Successful circularization and trafficking of the linear fusion RNA throughout the plant is evidenced by circular RNA transcripts in more distal plant structures. qRT-PCR is used to quantify RNA transcripts of the fusion RNAs. Linear constructs are expected to have lower or absent fluorescence when compared with circular RNAs. Linear CircRNA4 containing the intact Broccoli aptamer is expected to have lower fluorescence than a circularized circRNA4. Additionally, efficiently circularized and trafficked RNA constructs can be visualized by green fluorescence in distal leaves by the emission of green fluorescence at 472 nm after incubation with the DFHBI-1T fluorogen. Fluorescence indicates both efficient circularization and trafficking of the circular fusion RNA.

### Example 26: PSTVd R+dsRNA to Target RPL7 in CBP

This example describes the utilization of the replication motif from a viroid to replicate an RNA molecule that includes an effector in a plant cell and then deliver to insects by ingestion. The effector targets insect endogenous gene and cause mortality or stunting in larvae of Leptinotarsa or other species In this example, the RNA vector includes the following:
replication domain of PSTVd (potato spindle tuber viroid), consisting of PSTVd TL-CCR (SEQ ID NO: 12): the left-terminal and central conserved region of PSTVd
effector: hpRNA-RPL7, hairpin RNA (SEQ ID NO: 898) targeting an endogenous gene, Ribosomal Protein L7, from Leptinotarsa decemlineata (Colorado potato beetle, CPB)

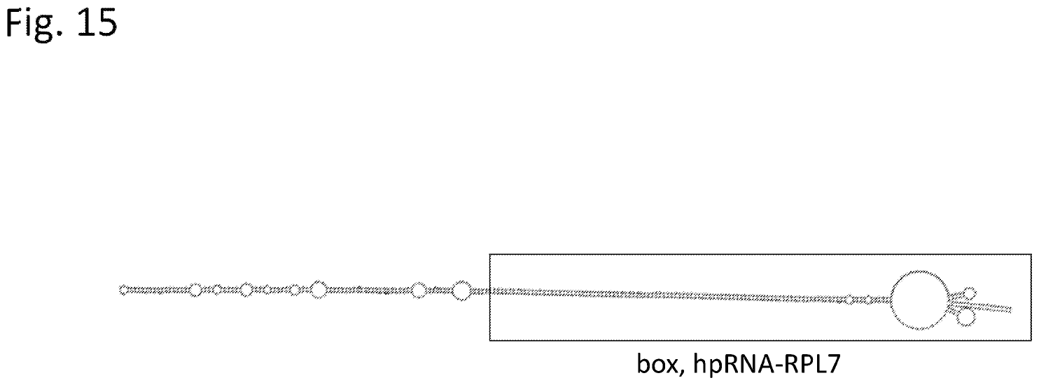

The resulting RNA vector (SEQ ID NO: 899), R-hpRNA-RPL7 (FIG. 15) targeting CPB RPL7 gene (NCBI Gene ID: 111514553) is tested using leaf disc assay to test mortality or stunting of CPB larvae.

### Design

The hpRNA targeting RPL7 (SEQ ID NO: 898) is derived from a hairpin dsRNA encoded by the DNA construct from SEQ ID NO:1105 of U.S. Pat. No. 9,777,288B2 with 90 nt targeting RPL7 gene flanking the loop region (149 nt). The hpRNA is fused to PSTVd TL-CCR. The RNA vector (SEQ ID NO: 899) is synthesized with in vitro transcription as described in Example 2. Two forms (linear and circular) of the RNA vector are synthesized and tested with leaf disc assay.

### Characterization

The synthesized linear and circular fusion RNAs are quantified using an Agilent 2100 Bioanalyzer system and analyzed using RNase H and RNase R assays, as described in Examples 6 and 7.

### Delivery and Bioassay

The RNA vector is added in a 0.1% Silwet® L77 solution in nuclease free water and then applied to potato leaves. The control leaves are treated with the formulation 0.1% Silwet® L77 solution. Treated leaves are collected at different time points, including 12 hours, 24 hours, 2 days, 4 days and 7 days. The replication of the RNA vector is measured by qPCR from leaf samples collected at different time points. Treated leaves are also cut into leaf discs and placed individually into wells of 128-well plate containing 0.5m/well of a solidified 2% agar. A single CPB neonate is placed in each well and incubated overnight to allow it to consume the leaf disc. The next day, CPB larvae are transferred to a feeding arena made from a covered, aerated 18-ounce translucent plastic container lined at its base with filter paper and containing potato foliage with stems inserted in a water-filled tube for freshness. The insects are incubated in the feeding arena in an environmental chamber (27 degrees Celsius; 60% relative humidity; 16 hours light/8 hours dark) with potato foliage replenished as needed. Larval viability is monitored daily and recorded as alive or dead for 16 days.

### Example 27: A Nuclear Transporter for Delivering an RNA Cargo or Effector to the Nucleus of a Cell

This example describes a method of delivering an RNA cargo that is carried by a synthetic viroid to a cell organelle. More specifically, this example illustrates the use of a synthetic or recombinant “nuclear transporter” polynucleotide to transport a heterologous effector or RNA cargo to the nucleus in a plant cell. In embodiments, such a synthetic nuclear transporter comprises (i) a single-stranded RNA (ssRNA) viroid sequence and (ii) a heterologous RNA sequence comprising or encoding an effector, wherein the ssRNA viroid sequence does not include a chloroplast localization sequence. In embodiments, the ssRNA viroid sequence is, or is derived from, the sequence of a viroid that replicates in a plant cell nucleus, such as a pospivirus, e.g., any of the pospiviroids identified by name and sequence identifier in Table 1 above.

In one experiment, the sequence of a longer-than-unit PSTVd (SEQ ID NO: 922) was employed as the scaffold for designing a synthetic nuclear transporter including a heterologous effector or RNA cargo to be transported into the nucleus of a plant cell. In related embodiments, other pospiviroid sequences are used as the scaffold for a synthetic nuclear transporter. In vitro synthesized nuclear transporters (SEQ ID NOs: 923 and 924) based on the longer-than-unit PSTVd scaffold modified to include a 21-nucleotide Solanum lycopersicum phytoene desaturase sequence (SEQ ID NO:9) as a heterologous effector or RNA cargo was produced using methods previously described (Examples 1-4 and 8) and Cy3-labeled using HyperScribe T7 High Yield Cy3 RNA Labeling Kit (APExBIO) following the manufacturer's protocol. Nicotiana tabacum BY2 protoplasts were isolated and transfected as described in Example 9. Ten μg of a Cy3-labeled synthetic nuclear transporter carrying the PDS sequence as an RNA cargo were transfected into 200 μL of 1×10⁶/mL BY2 protoplasts. After transfection, protoplasts were kept in the dark and incubated at room temperature for five hours. The protoplasts were then stained for 30 minutes with Hoechst 33342 dye (ThermoFisher, final concentration of 20 μg/mL), followed by imaging with an Olympus IX83 fluorescence microscope. Hoechst 33342 was visualized with a DAPI filter for nuclear localization. Cy3 was visualized with an RFP filter to identify areas in the cell to which the synthetic nuclear transporter carrying the cargo RNA had localized. The Cy3 signals were co-localized with the Hoechst 33342 signals, indicating that the synthetic nuclear transporter and its RNA cargo had localized to nucleus. These results demonstrate that a synthetic nuclear transporter based on a viroid scaffold sequence and including a heterologous effector or RNA cargo can transport the heterologous effector or RNA cargo to the nucleus of a plant cell.

In other embodiments, synthetic nuclear transporters, such as synthetic viroids based on PSTVd similar to the one described above, are used to transport at least one heterologous effector or RNA cargo to a predetermined subcellular location or organelle (in this case, the nucleus) in a plant cell. Such synthetic nuclear transporters are useful for delivering diverse heterologous effectors or RNA cargoes, such as, but not limited to, one or more small RNAs (e.g., siRNAs, trans-acting siRNAs, miRNAs, crRNAs, guide RNAs, or precursors of any of these), tRNAs or tRNA-like motifs, RNA aptamers, or combinations of any of these or other RNA cargoes to the nucleus of a plant; see also, e.g., Examples 14, 18, and 26, which further illustrate incorporation of a heterologous RNA sequence into a viroid-derived scaffolds. In embodiments, at least one synthetic nuclear effector that includes one or more heterologous effectors or RNA cargoes is co-delivered with a polypeptide, e.g., a nuclease or a ligase. In an embodiment, a synthetic nuclear transporter carrying a heterologous effector or RNA cargo that includes two guide RNAs in tandem is co-delivered with a Cas nuclease to a plant cell. In another embodiment, a synthetic nuclear transporter carrying a heterologous effector or RNA cargo including an siRNA or siRNA precursor (e.g., a hairpin) or an miRNA or miRNA precursor (e.g., an engineered miRNA precursor) is delivered to a plant, e.g., topically applied to the surface of a plant or injected into a plant's vascular system for systemic delivery or delivery to other parts of the plant; specific embodiments include those where the siRNA or siRNA precursor or an miRNA or miRNA precursor targets a gene of a plant pest or pathogen. In an example, a synthetic nuclear transporter based on a longer-than-unit PSTVd scaffold (SEQ ID NO: 922) is designed to include as the heterologous effector or RNA cargo at least one hairpin RNA (SEQ ID NO: 898) targeting the endogenous gene, Ribosomal Protein L7, from Leptinotarsa decemineata (Colorado potato beetle, CPB); in other embodiments, other siRNA or siRNA precursors or miRNA or miRNA precursors that target an essential gene of a plant pest or pathogen are similarly used.

In embodiments, the synthetic nuclear transporter comprises (i) a single-stranded RNA (ssRNA) viroid sequence and (ii) a heterologous RNA sequence comprising or encoding an effector, wherein the ssRNA viroid sequence does not include a chloroplast localization sequence. In embodiments, the ssRNA viroid is a pospiviroid, e.g., any of the pospiviroids identified by name and sequence identifier in Table 1 above.

In embodiments, the ssRNA viroid has a sequence having at least 80% sequence, at least 85%, at least 90%, at least 95%, at least 98%, at least 99%, or 100% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467. In embodiments, the ssRNA viroid has a sequence having at least 80% sequence, at least 85%, at least 90%, at least 95%, at least 98%, at least 99%, or 100% sequence identity to SEQ ID NO:51.

### Other Embodiments

Some embodiments of the technology described herein can be defined according to any of the following numbered embodiments:

1. A composition comprising a recombinant polynucleotide comprising: (i) a single-stranded RNA (ssRNA) viroid sequence and (ii) a heterologous RNA sequence comprising or encoding an effector, the composition being formulated for topical delivery to a plant.

2. The composition of paragraph 1, wherein the ssRNA viroid sequence is a viroid genome or a derivative thereof.

3. The composition of paragraph 1, wherein the ssRNA viroid sequence is a viroid genome fragment or a derivative thereof.

4. The composition of any one of paragraphs 1-3, wherein the recombinant polynucleotide encodes at least two ssRNA viroid sequences.

5. The composition of any one of paragraphs 1-4, wherein the topical delivery is spraying, leaf rubbing, soaking, coating, injecting, seed coating, or delivery through root uptake.

6. The composition of any one of paragraphs 1-5, further comprising an additional formulation component.

7. The composition of any one of paragraphs 1-5, wherein the composition does not comprise an additional formulation component.

8. The composition of any one of paragraphs 1-7, wherein the ssRNA viroid sequence comprises a sequence of at least 40 ribonucleotides which is at least 80% identical to a sequence, or fragment thereof, listed in Table 1.

9. The composition of paragraph 8, wherein the ssRNA viroid sequence has at least 90% identity to a sequence of Table 1.

10. The composition of paragraph 9, wherein the ssRNA viroid sequence has at least 95% identity to a sequence of Table 1.

11. The composition of paragraph 10, wherein the ssRNA viroid sequence has at least 98% identity to a sequence of Table 1.

12. The composition of paragraph 11, wherein the ssRNA viroid sequence has at least 99% identity to a sequence of Table 1.

13. The composition of any one of paragraphs 8-12, wherein the sequence of Table 1 is SEQ ID NO: 50.

14. The composition of any one of paragraphs 8-12, wherein the sequence of Table 1 is SEQ ID NO: 51.

15. The composition of any one of paragraphs 1-7, wherein the viroid is from the family Pospiviroidae or Avsunviroidae.

16. The composition of any one of paragraphs 1-7 and 15, wherein the viroid is eggplant latent viroid (ELVd), potato spindle tuber viroid (PSTVd), hop stunt viroid, coconut cadang-cadang viroid, apple scar skin viroid, Coleus blumei viroid 1, avocado sunbiotch viroid, peach latent mosaic viroid, chrysanthemum chlorotic mottle viroid, or Dendrobium viroid.

17. The composition of paragraph 16, wherein the viroid is PSTVd.

18. The composition of paragraph 16, wherein the viroid is ELVd.

19. The composition of any one of paragraphs 1-7, wherein the ssRNA viroid sequence comprises a sequence that is at least 80% identical to a sequence listed in Table 2 or Table 3.

20. The composition of paragraph 19, wherein the ssRNA viroid sequence has at least 90% identity to a sequence of Table 2 or Table 3.

21. The composition of paragraph 20, wherein the ssRNA viroid sequence has at least 95% identity to a sequence of Table 2 or Table 3.

22. The composition of paragraph 21, wherein the ssRNA viroid sequence has at least 98% identity to a sequence of Table 2 or Table 3.

23. The composition of paragraph 22, wherein the ssRNA viroid sequence has at least 99% identity to a sequence of Table 2 or Table 3.

24. The composition of paragraph 4, wherein each of the at least two ssRNA viroid sequences are at least 80% identical to a sequence listed in Table 2 or Table 3.

25. The composition of paragraph 24, wherein the recombinant polynucleotide encodes a sequence that is at least 80% identical to SEQ ID NO: 884 and encodes a sequence that is at least 80% identical to SEQ ID NO: 885.

26. The composition of paragraph 24, wherein the recombinant polynucleotide encodes a sequence that is at least 80% identical to SEQ ID NO: 886 and encodes a sequence that is at least 80% identical to SEQ ID NO: 887.

27. The composition of paragraph 24, wherein the recombinant polynucleotide encodes a sequence that is at least 80% identical to SEQ ID NO: 888 and encodes a sequence that is at least 80% identical to SEQ ID NO: 889.

28. The composition of paragraph 24, wherein the recombinant polynucleotide encodes a sequence that is at least 80% identical to SEQ ID NO: 890 and encodes a sequence that is at least 80% identical to SEQ ID NO: 891.

29. The composition of paragraph 24, wherein the recombinant polynucleotide encodes a sequence that is at least 80% identical to SEQ ID NO: 892 and encodes a sequence that is at least 80% identical to SEQ ID NO: 893.

30. The composition of any one of paragraphs 24-29, wherein the recombinant polynucleotide comprises 3, 4, 5, 6, 7, 8, 9, 10, or more than 10 ssRNA viroid sequences that are at least 80% identical to a sequence listed in Table 2 or Table 3.

31. The composition of any one of paragraphs 1-30, wherein the ssRNA viroid sequence comprises, in secondary structure, one or more of a replication motif, a transmission motif, a targeting motif, or a binding motif.

32. The composition of any one of paragraphs 1-25 and 27-31, wherein the ssRNA viroid sequence does not contain a pathogenicity domain.

33. The composition of any one of paragraphs 1-32, wherein the ssRNA viroid sequence comprises an internal loop, a stem-loop, a bulge loop, or a pseudoknot.

34. The composition of any one of paragraphs 1-33, wherein the ssRNA viroid sequence comprises a replication domain, a transmission domain, a targeting domain, or a binding domain.

35. The composition of paragraph 34, wherein the transmission domain is a tissue transmission domain, a cell-cell transmission domain, or a subcellular transition domain.

36. The composition of paragraph 34, wherein the targeting domain is a tissue targeting domain, a cell targeting domain, or a subcellular targeting domain.

37. The composition of paragraph 34 or 36, wherein the targeting domain binds to a host cell.

38. The composition of paragraph 34 or 36, wherein the targeting domain is a nuclear targeting sequence or a nuclear exclusion sequence.

39. The composition of paragraph 34, wherein the binding domain binds a molecular target in the plant.

40. The composition of paragraph 39, wherein the binding domain binds DICER.

41. The composition of any one of paragraphs 1-40, wherein the RNA sequence comprising or encoding the effector is not a viroid sequence and has a biological effect on a plant.

42. The composition of any one of paragraphs 1-41, wherein the effector comprises or is encoded by an ssRNA sequence.

43. The composition of any one of paragraphs 1-42, wherein the effector comprises a coding sequence.

44. The composition of paragraph 43, wherein the coding sequence encodes a protein or a polypeptide.

45. The composition of paragraph 42, wherein the effector is a regulatory RNA.

48. The composition of paragraph 45, wherein the regulatory RNA is a lncRNA, circRNA, tRF, tRNA, rRNA, snRNA, snoRNA, or piRNA.

47. The composition of paragraph 42, wherein the effector is an interfering RNA. 48. The composition of paragraph 47, wherein the effector is a dsRNA or a hpRNA.

49. The composition of paragraph 47, wherein the effector is a microRNA (miRNA) or a pre-miRNA. 50. The composition of paragraph 47, wherein the effector is a phasiRNA. 51. The composition of paragraph 47, wherein the effector is a hcsiRNA.

52. The composition of paragraph 47, wherein the effector is a natsiRNA.

53. The composition of paragraph 42, wherein the effector is a guide RNA.

54. The composition of any one of paragraphs 1-53, wherein the effector binds a target host cell factor.

55. The composition of paragraph 54, wherein the target host cell factor is a nucleic acid, a protein, a DNA, or an RNA.

56. The composition of any one of paragraphs 1-55, wherein the recombinant polynucleotide further comprises an internal ribosome entry site (IRES), a 5′ homology arm, a 3′ homology arm, a polyadenylation sequence, a group I permuted intron-exon (PIE) sequence, an RNA cleavage site, a ribozyme, a DICER-binding sequence, an mRNA fragment comprising an intron, an exon, a combination of one or more introns and exons, an untranslated region (UTR), an enhancer region, a Kozak sequence, a start codon, or a linker.

57. The composition of paragraph 56, wherein the ribozyme is a hammerhead ribozyme, a riboswitch, or a twister/tornado.

58. The composition of paragraph 56, wherein the DICER-binding sequence flanks the effector.

59. The composition of any one of paragraphs 56-58, wherein the recombinant polynucleotide comprises 2, 3, 4, 5, 6, 7, 8, 9, 10, or more than 10 additional heterologous sequence elements.

60. The composition of any one of paragraphs 1-59, wherein the recombinant polynucleotide lacks free ends.

61. The composition of paragraph 60, wherein the recombinant polynucleotide is circular.

62. The composition of any one of paragraphs 1-59, wherein the recombinant polynucleotide comprises at least one free end.

63. The composition of any one of paragraphs 1-62, wherein the recombinant polynucleotide is concatemeric.

64. The composition of any one of paragraphs 1-62, wherein the recombinant polynucleotide is linear.

65. A cell comprising the composition of any one of paragraphs 1-64.

66. The cell of paragraph 65, wherein the cell is a plant cell.

67. The cell of paragraph 66, wherein the plant cell is a monocot cell or a dicot cell.

68. The cell of paragraph 66, wherein the plant cell is a protoplast.

69. The cell of any one of paragraphs 65-68, wherein the cell has been transiently transformed with the recombinant polynucleotide.

70. The cell of any one of paragraphs 65-68, wherein the cell has been stably transformed with the recombinant polynucleotide.

71. The composition of any one of paragraphs 1-84, further comprising a plant cell.

72. A liposome comprising the composition of any one of paragraphs 1-64.

73. A vesicle comprising the composition of any one of paragraphs 1-64.

74. A formulation comprising the composition of any one of paragraphs 1-84.

75. The formulation of paragraph 74, wherein the formulation is a liquid, a gel, or a powder.

76. The formulation of paragraph 74 or 75, wherein the formulation is configured to be sprayed on plants, to be rubbed on leaves, to be coated on seeds, or to be delivered to roots.

77. A method of delivering an effector to a plant, a plant tissue, or a plant cell, comprising providing to a plant, plant tissue, or plant cell a composition of any one of paragraphs 1-84, whereby the effector comprised by or encoded by the heterologous RNA sequence is delivered to the plant, plant tissue, or plant cell.

78. The method of paragraph 77, wherein the plant is a monocot or a dicot.

79. The method of paragraph 77, wherein the plant cell is a protoplast.

80. The method of any one of paragraphs 77-79, wherein providing the composition to the plant, plant tissue, or plant cell comprises delivering the composition to a leaf, root, stem, flower, seed, xylem, phloem, apoplast, symplast, meristem, fruit, embryo, microspore, pollen, pollen tube, ovary, ovule, or explant for transformation of the plant.

81. The method of paragraph 80, wherein the fruit is a pre-harvest fruit.

82. The method of paragraph 80, wherein the fruit is a post-harvest fruit.

83. A method of modifying a trait, phenotype, or genotype in a plant cell, comprising providing to the plant cell a composition of any one of paragraphs 1-84.

84. The method of paragraph 83, wherein modifying comprises expressing in the plant a heterologous protein encoded by the RNA sequence comprising or encoding an effector.

85. The method of paragraph 83, wherein modifying comprises reducing expression of a target gene of the plant.

86. The method of paragraph 83, wherein modifying comprises increasing expression of a target gene of the plant.

87. The method of paragraph 83, wherein modifying comprises editing a target gene of the plant.

88. The method of paragraph 83, wherein modifying comprises regulating a target gene in the plant.

89. The method of any one of paragraphs 83-88, wherein the ssRNA viroid sequence effects one or more results selected from the group consisting of entry into a tissue or cell of the plant; transmission through a tissue or cell or subcellular component of the plant; replication in a tissue or cell of the plant; targeting to a tissue or cell of the plant; and binding to a factor in a tissue or cell of the plant.

90. A composition comprising a recombinant polynucleotide comprising: (i) a single-stranded RNA (ssRNA) viroid sequence and (ii) a heterologous RNA sequence comprising or encoding an effector, wherein the ssRNA viroid sequence does not include a chloroplast localization sequence.

91. A method of delivering an RNA effector to the nucleus of a plant cell, comprising contacting a plant cell with a synthetic nuclear transporter comprising: (i) a single-stranded RNA (ssRNA) viroid sequence and (ii) a heterologous RNA sequence comprising or encoding an effector, wherein the ssRNA viroid sequence does not include a chloroplast localization sequence;
wherein the synthetic nuclear transporter localizes to the nucleus of the plant cell, thereby delivering the effector to the nucleus.

92. The method of paragraph 91, wherein the ssRNA viroid sequence has at least 80% sequence identity with a pospiviroid sequence.

93. The method of paragraph 91, wherein the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467.

94. The method of paragraph 91, wherein the ssRNA viroid sequence has at least 90% sequence identity with SEQ ID NO:51.

95. The method of paragraph 91, wherein the heterologous RNA sequence comprises coding RNA, non-coding RNA, or both coding and non-coding RNA.

96. The method of paragraph 91, wherein the effector comprises coding RNA, non-coding RNA, or both coding and non-coding RNA.

97. The method of paragraph 91, wherein the effector comprises non-coding RNA comprising at least one regulatory RNA or at least one interfering RNA that targets a transcript in a cell.

98. The method of paragraph 97, wherein the cell is selected from the group consisting of a plant cell, an arthropod cell, a mollusk cell, a fungus cell, or a nematode cell.

99. A composition comprising a synthetic nuclear transporter, wherein the synthetic nuclear transporter comprises: ( ) a single-stranded RNA (ssRNA) viroid sequence and (ii) a heterologous RNA sequence comprising or encoding an effector, wherein the ssRNA viroid sequence does not include a chloroplast localization sequence.

100. The composition of paragraph 96, wherein the ssRNA viroid sequence has at least 80% sequence identity with a pospiviroid sequence.

101. The composition of paragraph 96, wherein the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467.

102. The composition of paragraph 96, wherein the ssRNA viroid sequence has at least 90% sequence identity with SEQ ID NO:51.

103. The composition of paragraph 96, wherein the heterologous RNA sequence comprises coding RNA, non-coding RNA, or both coding and non-coding RNA.

104. The composition of paragraph 96, wherein the effector comprises coding RNA, non-coding RNA, or both coding and non-coding RNA.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Other embodiments are within the claims.

### Appendix 1. Plant Viroid NCBI References

MK303580.1, MK303579.1, MH025994.1, MF770204.1, MF770203.1, MF770202.1, MF770201.1, MF770200.1, MF770199.1, MF770197.1, MF882925.1, MF882924.1, KY522741.1, KY522740.1, KY522739.1, KY522738.1, KY522737.1, KY522736.1, KY522735.1, KY522734.1, KY522733.1, KY522732.1, KY522731.1, KY522730.1, KY522729.1, KY522728.1, MH476293.1, MH476292.1, MH476291.1, MH771135.1, LC433633.1, LC433632.1, LC433631.1, LC433630.1, LC433629.1, LC433628.1, LC430901.1, LQ907271.1, MH758760.1, MF770198.1, MF770196.1, LC388862.1, LC388861.1, LC388860.1, LC388859.1, LC388858.1, LC388857.1, LC388856.1, LC388855.1, LC388854.1, LC388853.1, LC388852.1, NC_027432.1, NC_011590.1, NC_003637.1, NC_003613.1, NC_003538.1, NC_002030.1, NC_002015.1, NC_000885.1, NC_001484.1, NC_001558.1, NC_001553.1, MG450358.1, MG450357.1, MG132059.1, MG132058.1, MG132057.1, MG132056.1, MG132055.1, S79831.1, KM214229.1, KM214228.1, KM214227.1, KM214226.1, KM214225.1, KM214224.1, KM214223.1, KM214222.1, KM214221.1, KM214220.1, KM214219.1, KM214218.1, KM214217.1, KM214216.1, KM214215.1, KM214214.1, KM214213.1, KM214212.1, KM214211.1, KM214210.1, KM214209.1, KM214208.1, KM214207.1, KC143305.1, KC143304.1, KC143303.1, KC143302.1, KC143301.1, KC143300.1, KC143299.1, KC143298.1, KC143297.1, KC143296.1, KC143295.1, KC143294.1, KC143293.1, KC143292.1, KC143291.1, KC143290.1, KC143289.1, JF742641.1, JF742640.1, JF742639.1, JF742638.1, JF742637.1, JF742636.1, JF742635.1, JF742634.1, JF742633.1, JF742632.1, DQ923061.1, DQ923060.1, DQ923059.1, DQ923058.1, DQ076250.1, DQ061193.1, DQ061192.1, DQ022677.1, KY936885.1, KY936884.1, KY936883.1, KY936882.1, KY936881.1, KY936880.1, KY936879.1, KY936878.1, KY936877.1, KY936876.1, KY936875.1, KY936874.1, KY936873.1, MG470649.1, KY110722.1, KY110721.1, MF803029.1, KY646193.1, KY328288.1, KY328287.1, KY810784.1, KY810771.1, MF140254.1, MF140253.1, MF140252.1, MF140251.1, MF140250.1, MF140249.1, MF140248.1, MF140247.1, MF140246.1, MF359723.1, MF359722.1, MF359721.1, MF359720.1, MF359719.1, MF359718.1, MF359717.1, MF359716.1, MF359715.1, MF359714.1, MF359713.1, MF359712.1, MF359711.1, MF359710.1, MF359709.1, MF359708.1, MF359707.1, MF359706.1, MF359705.1, MF359704.1, MF359703.1, MF359702.1, MF359701.1, MF359700.1, MF359699.1, MF359698.1, MF359697.1, MF359696.1, MF359695.1, MF359694.1, MF359693.1, MF359692.1, KY654688.1, KY235369.1, KY473932.1, KY473931.1, KX579067.1, KX819237.1, KX819236.1, KX819235.1, KX819234.1, KX819233.1, KX819232.1, KX819231.1, KX159284.1, KX159283.1, KX159282.1, KX159281.1, KX156933.1, KX156932.1, KX156931.1, KX156930.1, LC192498.1, LC192497.1, LC192496.1, LC192495.1, LC192494.1, LC192493.1, LC192492.1, LC192491.1, LC192490.1, LC192489.1, LC192488.1, LC192487.1, LC192486.1, LC192485.1, LC192484.1, LC192483.1, LC192482.1, LC192481.1, LC192480.1, LC192479.1, LC192478.1, LC192477.1, LC192476.1, LC192475.1, LC192474.1, LC192473.1, LC192472.1, LC192471.1, LC192470.1, LC192469.1, LC192468.1, LC192467.1, LC192466.1, LC192465.1, LC192464.1, LC192463.1, LC192462.1, LC192461.1, LC192460.1, LC192459.1, LC192458.1, LC192457.1, LC192456.1, LC192455.1, KX096576.1, KX096575.1, KX096574.1, KX096573.1, KX096572.1, KX096571.1, KX096570.1, KX096569.1, KX096568.1, KX096567.1, KX096566.1, KX096565.1, KX096564.1, KX096563.1, KX096562.1, KX096561.1, KX096560.1, KX096559.1, KX096558.1, KX096557.1, KX096556.1, KX096555.1, KX096554.1, KX096553.1, KX096552.1, KX096551.1, KX096550.1, KX096549.1, KX096548.1, KX096547.1, KX096546.1, KX096545.1, KX096544.1, KX096543.1, KX096542.1, KX096541.1, KX096540.1, KX096539.1, KX096538.1, KX096537.1, KX096536.1, KX096535.1, KX096534.1, KX096533.1, KX096532.1, KX096531.1, KX096530.1, KX096529.1, KX096528.1, KX096527.1, KX096526.1, KX096525.1, KX096524.1, KX096523.1, KX096522.1, KX096521.1, KX096520.1, KX096519.1, KX096518.1, KX096517.1, KX096516.1, KX096515.1, KX096514.1, KX096513.1, KX096512.1, KX096511.1, KX096510.1, KX096509.1, KX096508.1, KX096507.1, KX096506.1, KX096505.1, KX096504.1, KX096503.1, KX096502.1, KX096501.1, KX096500.1, KX096499.1, KX096498.1, KX096497.1, KX096496.1, KX096495.1, KX096494.1, KX096493.1, KX096492.1, KX096491.1, KX096490.1, KX096489.1, KX096488.1, KX096487.1, KX096486.1, KX096485.1, KX096484.1, KX096483.1, KX096482.1, KX096481.1, KX096480.1, KX096479.1, KX096478.1, KX096477.1, KX096476.1, KX096475.1, KX096474.1, KX096473.1, KX096472.1, KX096471.1, KX096470.1, KX096469.1, KX096468.1, KX096467.1, KX096466.1, KX096465.1, KX096464.1, KX096463.1, KX096462.1, KX096461.1, KX096460.1, KX096459.1, KX096458.1, KX096457.1, KX096456.1, KX096455.1, KX096454.1, KX096453.1, KX096452.1, KX096451.1, KX096450.1, KX096449.1, KX096448.1, KX096447.1, KX096446.1, KX096445.1, KX096444.1, KX096443.1, KX096442.1, KX096441.1, KX096440.1, KX096439.1, KX096438.1, KX096437.1, KX096436.1, KX096435.1, KX096434.1, KX096433.1, KX096432.1, KX096431.1, KX096430.1, KX096429.1, KX096428.1, KX096427.1, KX096426.1, KX096425.1, KX096424.1, KX096423.1, KX096422.1, KX096421.1, KX096420.1, KX096419.1, KX096418.1, KX096417.1, KX096416.1, KX096415.1, KX096414.1, KX096413.1, KX096412.1, KX096411.1, KX096410.1, KX096409.1, KX096408.1, KX096407.1, KX096406.1, KX096405.1, KX096404.1, KX096403.1, KX096402.1, KX096401.1, KX096400.1, KX096399.1, KX096398.1, KX096397.1, KX096396.1, KX096395.1, KX096394.1, KX096393.1, KX096392.1, KX096391.1, KX096390.1, KX096389.1, KX096388.1, KX096387.1, KX096386.1, KX096385.1, KX096384.1, KX096383.1, KX096382.1, KX096381.1, KX096380.1, KX096379.1, KX096378.1, KX096377.1, KX096376.1, KX096375.1, KX096374.1, KX096373.1, KX096372.1, KX096371.1, KX096370.1, KX096369.1, KX096368.1, KX096367.1, KX096366.1, KX096365.1, KX096364.1, KX096363.1, KX096362.1, KX096361.1, KX096360.1, KX096359.1, KX096358.1, KX096357.1, KX096356.1, KX096355.1, KX096354.1, KX096353.1, KX096352.1, KX096351.1, KX096350.1, KX096349.1, KX096348.1, KX096347.1, KX370618.1, KM204319.1, KM204318.1, KM204317.1, KX618912.1, KX084712.1, KX084711.1, KX084710.1, KX084709.1, KX084708.1, KU714937.1, KU714936.1, KU714935.1, KU714934.1, KP454054.1, KP454053.1, KP454052.1, KP454051.1, KP454050.1, KP454049.1, KP454048.1, KP454047.1, KP454046.1, KP454045.1, KP454044.1, KP454043.1, KP454042.1, KP454041.1, KP454040.1, KP454039.1, KP454038.1, KP454037.1, KJ857498.1, KJ857497.1, KJ857496.1, JX259396.1, JX259395.1, JX259394.1, JX259393.1, JX259392.1, KT923159.1, LC093172.1, LC093171.1, LC093170.1, LC093169.1, KU323793.1, KU323792.1, KU323791.1, KU323790.1, KT987925.1, KU535585.1, KU535584.1, KU535583.1, KU535582.1, KU535581.1, KU535580.1, KU535579.1, KP863712.1, KP863711.1, KP863710.1, KP759284.1, KP759283.1, KT725630.1, LC123589.1, HG739074.1, LC062712.1, LC062713.1, KR020041.1, KR020040.1, KR020039.1, KR020038.1, KR020037.1, KR611376.1, KR611375.1, KR611374.1, KR611373.1, KR611372.1, KR611371.1, KR611370.1, KR611369.1, KR611368.1, KR611367.1, KR611366.1, KR611365.1, KR611364.1, KR611363.1, KR611362.1, KR611361.1, KR611360.1, KR611359.1, KR611358.1, KR611357.1, KR611356.1, KR611355.1, KR611354.1, KR611353.1, KR611352.1, KR611351.1, KR611350.1, KR611349.1, KR611348.1, KR611347.1, KR611346.1, KR611345.1, KR611344.1, KR611343.1, KR611342.1, KR611341.1, KR611340.1, KR611339.1, KR611338.1, KR611337.1, KR611336.1, KR611335.1, KR611334.1, LC090814.1, LC090813.1, AB279771.1, AB279770.1, AB279769.1, AB279768.1, LC088323.1, LC088322.1, KT005827.1, KT005826.1, KT005825.1, KT005824.1, KT005823.1, KT005822.1, KT005821.1, KT005820.1, KT005819.1, KT005818.1, KT005817.1, KT005816.1, KT005815.1, KT005814.1, KT005813.1, KT005812.1, KT005811.1, KT005810.1, KT005809.1, KT005808.1, KT005807.1, KT005806.1, KT005805.1, KT005804.1, KT005803.1, KR677387.1, HF954920.1, HF954919.1, HF954918.1, HF954917.1, HF954916.1, KF683201.1, KF683199.1, KF683198.1, KF484878.1, KF265340.1, KP262537.1, KP262536.1, KP262535.1, KP262534.1, KJ956803.1, KJ956802.1, KJ956801.1, KJ956800.1, KJ956799.1, KJ956798.1, KJ810544.1, KJ810543.1, KC290928.1, KC290927.1, JX909290.1, LN681196.1, KJ635807.1, KM588065.1, D1377399.1, HG739082.1, HG739081.1, HG739080.1, HG739079.1, HG739078.1, HG739077.1, HG739076.1, HG739075.1, HG739073.1, HG739072.1, HG739071.1, HG739070.1, KJ817885.1, HG916812.1, KF418774.1, KF418773.1, KF418772.1, KF418771.1, KF418770.1, KF418769.1, KF418768.1, KF418767.1, KF437621.1, KJ810547.1, KJ810546.1, KJ810545.1, KJ538556.1, KJ538555.1, KJ538554.1, JQ904597.1, KF683200.1, KF973205.1, HG916813.1, FR851463.1, KF049417.1, KC282384.1, KC282383.1, KC282382.1, KC282381.1, KF419168.1, KC707563.1, EU879926.1, EU879925.1, EU879924.1, EU879923.1, EU879922.1, EU879921.1, EU879920.1, EU879919.1, EU879918.1, EU879917.1, EU879916.1, EU879915.1, EU879914.1, EU879913.1, KF484879.1, KF493732.1, KC762954.1, KC762953.1, KC762952.1, KF021223.1, KC810010.1, KC810009.1, KC810008.1, KC810007.1, KC810006.1, KC810005.1, AB746838.1, KC707564.1, JQ362419.1, JQ809268.1, JQ809267.1, JQ809266.1, JQ809265.1, JQ809264.1, JQ809263.1, JQ809262.1, JQ809261.1, JQ809260.1, JQ809259.1, JQ809258.1, JQ809257.1, JQ809256.1, JQ809255.1, JQ809254.1, JQ809253.1, JQ809252.1, JX945671.1, KC345440.1, JX280944.1, JX885867.1, JX885866.1, JX885865.1, JX885864.1, JX867556.1, JX237458.1, JX237457.1, JQ975098.1, EU872276.1, HQ284030.1, HQ284029.1, HQ284028.1, HQ284027.1, HQ284026.1, HQ284025.1, HQ284024.1, HQ284023.1, HQ284022.1, HQ284021.1, HQ284020.1, HQ284019.1, HQ284018.1, HQ284017.1, HQ284016.1, HQ284015.1, HQ284014.1, HQ284013.1, HQ284012.1, HQ284011.1, JQ974379.1, JQ974378.1, JQ904911.1, JQ889854.1, JQ889853.1, JQ889852.1, JQ889851.1, JQ889850.1, JQ889849.1, JQ889848.1, JQ889847.1, JQ889846.1, JQ889845.1, JQ889844.1, JQ889843.1, JQ889842.1, JQ889841.1, JQ889840.1, JQ889839.1, JQ806338.1, JQ685742.1, JQ685741.1, JQ685740.1, JQ685739.1, JQ685738.1, JQ685737.1, JQ685736.1, JQ685735.1, JQ685734.1, JQ889690.1, JQ889689.1, JN872140.1, JF927790.1, JF414238.1, AB679214.1, AB679213.1, AB679212.1, AB679211.1, AB679210.1, AB679209.1, AB679208.1, AB679207.1, AB679206.1, AB679205.1, AB679204.1, AB679203.1, AB679202.1, AB679201.1, AB679200.1, AB679199.1, AB679198.1, AB679197.1, AB679196.1, AB679195.1, AB679194.1, AB679193.1, JN872144.1, JN872143.1, JN872142.1, JN872141.1, JQ083647.1, JQ083646.1, JQ083645.1, JN543957.1, JN559764.1, JN559763.1, AB689034.1, AB689033.1, AB689032.1, AB689031.1, HE575349.1, HQ667141.1, HQ667140.1, HQ667139.1, HQ667138.1, GQ229575.1, JA451622.1, JA451621.1, GU295988.1, JF938538.1, AB623143.1, HQ891019.1, HQ891018.1, HQ639701.1, HQ639700.1, HQ639699.1, HQ639698.1, HQ639697.1, FN994891.1, FW588639.1, JF446939.1, JF446938.1, JF446937.1, JF446936.1, JF446935.1, JF446934.1, JF446933.1, JF446932.1, JF446931.1, JF446930.1, JF446929.1, JF446928.1, JF446927.1, JF446926.1, JF446925.1, JF446924.1, JF446923.1, JF446922.1, JF446921.1, JF446920.1, JF446919.1, JF446918.1, JF446917.1, JF446916.1, JF446915.1, JF446914.1, JF446913.1, JF446912.1, JF446911.1, JF446910.1, JF446909.1, JF446908.1, JF446907.1, JF446906.1, JF446905.1, JF446904.1, JF446903.1, JF446902.1, JF446901.1, JF446900.1, JF446899.1, JF446898.1, JF446897.1, JF446896.1, JF446895.1, JF446894.1, JF446893.1, JF446892.1, JF446891.1, JF446890.1, JF446889.1, JF446888.1, JF446887.1, JF446886.1, JF446885.1, HQ731652.1, FN669540.2, FN669539.2, FN669538.2, FN669537.2, FN669536.2, FN669535.2, FN669534.2, FN669533.2, FN669532.2, FN669531.2, FN669530.2, GQ853469.1, GQ853468.1, GQ853467.1, GQ853466.1, GQ853465.1, GQ853484.1, GQ853463.1, GQ853462.1, GQ853461.1, GQ853460.1, GQ853459.1, GQ853458.1, GQ853457.1, GU300810.1, HQ456944.1, FJ409044.1, HQ423166.1, HQ454937.1, HQ454936.1, HQ454935.1, HQ454934.1, HQ454933.1, HQ454932.1, HQ454931.1, HQ454930.1, HQ454929.1, HQ454928.1, HQ454927.1, HQ454926.1, HQ454925.1, HQ454924.1, HQ454923.1, HQ454922.1, HQ454921.1, HQ454920.1, HQ454919.1, HQ454918.1, HQ454917.1, HQ454916.1, HQ454915.1, HQ454914.1, GU825981.1, GU825980.1, FJ751934.1, FJ751933.1, FJ751932.1, FJ751931.1, FJ751930.1, FJ751929.1, FJ751928.1, FJ751927.1, FJ751926.1, FJ751925.1, FJ662762.1, EU382204.1, EU382203.1, EU382202.1, EU273604.1, EU257478.1, EF582393.1, EF582392.1, EF186991.1, EF186990.1, EF186989.1, EF126048.1, EF126047.1, DQ831486.1, DQ831485.1, HQ452417.1, HQ452416.1, HQ452415.1, HQ452414.1, HQ452413.1, HQ452412.1, HQ452411.1, HQ452410.1, HQ452409.1, HQ452408.1, HQ452407.1, HQ452406.1, HQ452405.1, HQ452404.1, HQ452403.1, HQ452402.1, HQ452401.1, HQ452400.1, HQ452399.1, HM753555.1, EF626530.1, EF488068.1, EF488067.1, EF488066.1, EF488065.1, EF488064.1, EF488063.1, EF488062.1, EF488061.1, EF488060.1, EF488059.1, EF488058.1, EF488057.1, EF488056.1, EF488055.1, EF488054.1, EF488053.1, EF488052.1, EF488051.1, EF488050.1, EF488049.1, AY937194.1, AY937193.1, AY937192.1, AY937191.1, AY937190.1, AY937189.1, AY937188.1, AY937187.1, AY937186.1, AY937185.1, AY937184.1, AY937183.1, AY937182.1, AY937181.1, AY937180.1, AY937179.1, HH970096.1, HH970094.1, HH970091.1, HM560977.1, GU481092.1, GU481091.1, GU481090.1, EF494697.1, EF494696.1, EF494695.1, EF494694.1, EF494693.1, EF494692.1, EF494691.1, EF494690.1, EF494689.1, EF494688.1, EF494687.1, EF494686.1, EF494685.1, EF494684.1, EF494683.1, EF494682.1, EF494681.1, EF494680.1, EF494679.1, EF494678.1, EF494677.1, EF051631.1, EF044305.1, EF044304.1, EF044303.1, EF044302.1, AM777161.1, AM774357.1, AM774356.1, EU094208.1, EU094207.1, FJ773255.1, FJ773254.1, FJ773253.1, EU862231.1, EU862230.1, GU911351.1, GU911350.1, EU729744.2, FJ797614.1, GU592444.1, DQ859013.1, FM995507.1, FM995506.1, FJ715463.1, FJ715462.1, HM043820.1, HM043819.1, HM043818.1, HM043817.1, HM043816.1, HM043815.1, HM043814.1, HM043813.1, HM043812.1, HM043811.1, HM043810.1, GQ131573.1, GQ131572.1, FJ822878.1, FJ822877.1, FJ824844.1, M93686.1, AF162131.1, AY372399.2, GQ169709.1, EF459701.1, EF459700.1, EF459699.1, EF459698.1, EF459697.1, FJ394072.1, FJ394071.1, FJ394070.1, AY222078.1, AY222077.1, AY222076.1, AY222075.1, AY222074.1, AY222073.1, AY222072.1, AF459007.1, AF459006.1, AF459005.1, AF459004.1, AF459003.1, AF459002.1, AF459001.1, AF459000.1, AF458999.1, AF458998.1, AF458997.1, AF458996.1, AF458995.1, AF458994.1, AF458993.1, AF458992.1, AF458991.1, AF458990.1, AF458989.1, AF458988.1, AF458987.1, AF458986.1, GQ396665.1, GQ396664.1, FN673554.1, FN673553.1, S67442.1, S67446.1, S67441.1, S67440.1, S67438.1, S67437.1, S52178.1, GQ915310.1, GQ260199.1, GQ260198.1, GQ260197.1, GQ260196.1, FN646407.1, GQ246194.1, GQ246192.1, GQ246191.1, GQ174502.1, GQ174501.1, AY492083.2, AY492084.1, AY492082.1, AY492081.1, AY492080.1, AY492079.1, AY492078.1, AY492077.1, AY492076.1, AY492075.1, U23060.1, U23059.1, U23058.1, M88678.1, EF580923.1, AY532801.1, AY373446.1, AY372400.1, AY372398.1, AY372397.1, AY372396.1, AY372395.1, AY372394.1, AY372393.1, AY372392.1, AY372391.1, AY372390.1, AY367350.1, AY365230.1, EU877746.1, EU877745.1, EU877744.1, EU877743.1, EU877742.1, EU625577.1, FJ904297.1, FJ904296.1, FJ904295.1, FJ904294.1, FJ904293.1, FJ904292.1, FJ872825.1, FJ872824.1, FJ872823.1, EU447280.1, M38345.1, FJ773261.1, FJ773260.1, FJ773259.1, FJ773258.1, FJ773257.1, FJ773256.1, FJ626866.1, FJ626865.1, FJ626864.1, FJ626863.1, FM998552.1, FM998551.1, FM998550.1, FM998549.1, FM998548.1, FM998547.1, FM998546.1, FM998545.1, FM998544.1, FM998543.1, FM998542.1, AB255880.1, AB255879.1, AB329668.1, X15663.1, EU926739.1, FJ031232.1, EU564185.1, EU564184.1, EU564183.1, EU564175.1, EU564174.1, EU564173.1, EU564172.1, EU564171.1, EU564170.1, EU564169.1, EF192396.2, EF192395.1, EF192394.1, EF192393.1, EU517117.1, EU512994.1, AM920649.1, AM774355.1, EU180221.1, DQ471996.1, DQ471995.1, DQ471994.1, DQ444474.1, DQ444473.1, EF551346.1, AM698095.1, AM698094.1, AM698093.1, DQ406591.1, DQ308561.1, DQ308560.1, DQ308559.1, DQ308558.1, DQ308557.1, DQ308556.1, DQ308555.1, DQ318794.1, DQ318793.1, DQ318792.1, DQ318791.1, DQ318790.1, X76845.1, X76846.1, X76844.1, X76848.1, X76847.1, EF015581.1, DQ846886.1, DQ846885.1, DQ846884.1, DQ846883.1, E00278.1, DQ431996.1, DQ431995.1, DQ431994.1, DQ431993.1, DQ431992.1, DQ431991.1, DQ400342.1, DD220190.1, DD220188.1, DD220185.1, DQ144506.1, DQ315388.1, X53716.1, AJ564803.1, AJ564802.1, AJ564801.1, AJ564800.1, AJ564799.1, AJ564798.1, AJ564797.1, AJ564796.1, AJ564795.1, X52040.1, X52039.1, X52038.1, X52037.1, X52036.1, DQ094298.1, DQ094297.1, DQ094296.1, DQ094295.1, DQ094294.1, DQ094293.1, AJ969017.1, AY962324.1, V01465.1, V01107.1, AY513268.1, AY671957.1, AY671956.1, AY671955.1, AY671954.1, AY671953.1, AY671952.1, X58388.1, AY673974.1, AJ634596.1, AY532804.1, AY532803.1, AY532802.1, AY523584.1, AY523583.1, AY523582.1, AY514447.1, AY514446.1, AY514444.1, AY518940.1, AY518939.1, AY517496.1, AY517495.1, AY517494.1, AY493560.1, AY493559.1, AY456136.1, AJ585258.1, AJ583449.1, AF540963.1, AF540962.1, AF540961.1, AF540960.1, AY360446.1, AY229990.1, X16409.1, X16408.1, X16407.1, D88895.1, AB006737.1, AY152841.1, AY152840.1, AJ515261.1, AF536193.1, AJ490825.1, AB054599.1, AB054598.1, AB054597.1, AB054596.1, AB054595.1, AB054594.1, AB054593.1, AB054592.1, Y00328.1, AY062121.1, AF483473.1, AF483472.1, AF483471.1, AF483470.1, M93685.1, E50939.1, AF458776.1, AF458775.1, AF458772.1, AF458771.1, AF454395.1, AF394453.1, AF394452.1, AF434678.1, AF428064.1, AF428063.1, AF428062.1, AF428061.1, AF428060.1, AF428059.1, AF428058.1, AF298178.1, AF298177.1, AF369530.1, AB055974.1, Y09382.1, Y09383.1, Y08852.1, Y09381.1, Y09577.1, Y09576.1, Y09575.1, Y09574.1, Y09889.1, Y09888.1, Y09887.1, Y09886.1, Y09890.1, Y09891.1, AF148717.1, AJ007489.1, L78463.1, L78462.1, L78461.1, L78460.1, L78459.1, L78458.1, L78457.1, L78456.1, L78454.1, U82445.1, AJ001853.1, AJ001852.1, AJ001851.1, AJ001850.1, AJ001849.1, AJ000046.1, X06390.1, X95292.1, X95293.1, X95734.1, X97387.1, Z34272.1, X17268.1, Z68201.1, U51895.1, U21126.1, M30870.1, M30869.1, M30871.1, M30868.1, K00817.1, K00818.1, M88677.1, M88681.1, M14814.1, M36163.1, M25199.1, M16826.1, M19506.1, M34917.1, K00965.1, K00964.1, J02053.1.

1: A method of delivering an effector to a eukaryote, comprising providing to the eukaryote a composition comprising a recombinant polynucleotide comprising: (i) a single-stranded RNA (ssRNA) viroid sequence and (ii) a heterologous RNA sequence comprising or encoding an effector, wherein the eukaryote is a plant, a fungus, or an animal and whereby the effector comprised by or encoded by the heterologous RNA sequence is delivered to the eukaryote.

2. (canceled)

3: The method of claim 1, wherein the composition is provided to a plant, plant tissue, or plant cell, or a processed product thereof, and wherein the eukaryote consumes or contacts the plant, plant tissue, or plant cell, or processed product thereof, whereby the effector is delivered to the eukaryote.

4: The method of claim 1, wherein (a) the ssRNA viroid sequence is a viroid genome or a derivative thereof or (b) the ssRNA viroid sequence is a viroid genome fragment or a derivative thereof.

5: The method of claim 1, wherein the ssRNA viroid sequence is a sequence of a viroid from the family Pospiviroidae or Avsunviroidae.

6: The method of claim 1, wherein the viroid is potato spindle tuber viroid (PSTVd) or eggplant latent viroid (ELVd).

7: The method of claim 1, wherein the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467.

8: The method of claim 1, wherein the ssRNA viroid sequence has at least 90% sequence identity to SEQ ID NO:51 or SEQ ID NO:50.

9: The method of claim 1, wherein the ssRNA viroid sequence does not contain a pathogenicity domain.

10: The method of claim 1, wherein the RNA sequence comprising or encoding the effector is not a viroid sequence and (a) has a biological effect on a plant or (b) has a biological effect on an animal or fungus that consumes or contacts the plant.

11: The method of claim 1, wherein the effector comprises or is encoded by an ssRNA sequence.

12: The method of claim 1, wherein the effector is an siRNA, miRNA, or pre-miRNA.

13: The method of claim 1, wherein the heterologous RNA sequence comprises coding RNA, non-coding RNA, or both coding and non-coding RNA.

14: The method of claim 1, wherein the effector comprises coding RNA, non-coding RNA, or both coding and non-coding RNA.

15: The method of claim 1 wherein the effector comprises non-coding RNA comprising at least one regulatory RNA or at least one interfering RNA that regulates a target gene or its transcript in a target cell.

16: The method of claim 15, wherein the target cell is selected from the group consisting of a plant cell, an animal cell, and a fungal cell.

17: The method of claim 15, wherein the effector modifies a trait, phenotype, or genotype in the target cell, optionally wherein the trait is pest resistance, fungal disease resistance, virus resistance, nematode resistance, bacterial disease resistance, or insect resistance.

18: The method of claim 17, wherein modifying comprises reducing expression of the target gene; increasing expression of the target gene; editing the target gene; and/or regulating the target gene.

19: The method of claim 1, wherein the ssRNA viroid sequence effects one or more results selected from the group consisting of entry into a tissue or cell of the eukaryote; transmission through a tissue or cell or subcellular component of the eukaryote; replication in a tissue or cell of the eukaryote; targeting to a tissue or cell of the eukaryote; and binding to a factor in a tissue or cell of the eukaryote.

20: The method of claim 1, wherein the recombinant polynucleotide lacks free ends and/or is circular.

21: The method of claim 1, wherein the composition is topically delivered to a plant a scion, or a rootstock, and wherein the topical delivery is spraying, leaf rubbing, soaking, coating, injecting, seed coating, or delivery through root uptake.

22: A composition comprising a recombinant polynucleotide comprising: (a) a single-stranded RNA (ssRNA) viroid sequence that is a viroid genome or a derivative thereof or a viroid genome fragment or a derivative thereof, and (b) a heterologous RNA sequence that is not a viroid sequence and comprises or encodes an effector.

23: The composition of claim 22, wherein the viroid genome is (a) a genome of a viroid from the family Pospiviroidae or Avsunviroidae, or (b) a genome of potato spindle tuber viroid (PSTVd) or eggplant latent viroid (ELVd).

24: The composition of claim 22, wherein the ssRNA viroid sequence has at least 80% sequence identity to a sequence selected from the group consisting of SEQ ID NOs:51-54, SEQ ID NOs:65-66, SEQ ID NO:68, SEQ ID NO:75, SEQ ID NOs:77-79, SEQ ID NOs:84-96, SEQ ID NOs:98-107, SEQ ID NOs:123-124, SEQ ID NOs:126-132, SEQ ID NO:134, SEQ ID NOs:136-143, SEQ ID NOs:145-150, SEQ ID NOs:153-154, SEQ ID NO:159, SEQ ID NO:166, SEQ ID NO:168, SEQ ID NO:196, SEQ ID NO:242, SEQ ID NO:268, SEQ ID NO:274, SEQ ID NO:276, SEQ ID NO:289, SEQ ID NO:451, SEQ ID NOs:458-459, and SEQ ID NO:467.

25: The composition of claim 22, wherein the ssRNA viroid sequence has at least 90% sequence identity to SEQ ID NO:51 or SEQ ID NO:50.

26: The composition of claim 22, wherein the effector comprises non-coding RNA comprising at least one regulatory RNA or at least one interfering RNA or at least one guide RNA that regulates or modifies a target gene or its transcript in a target cell, wherein the target cell is a plant cell, an animal cell, or a fungal cell.

27: The composition of claim 22, wherein the effector (a) modifies expression of a target gene in a eukaryotic cell; or (b) has a biological effect on a plant or on an animal or fungus that consumes or contacts the plant.

28: The composition of claim 22, wherein the composition is (a) formulated for delivery to a plant or to the environment in which the plant grows; or (b) formulated for delivery to an animal or fungus.
